(19) United States
(12) Patent Application Publication
(10) Pub. No.: US 2022/0304957 A1
(43) Pub. Date: Sep. 29, 2022

(54) ANTI-CANCER COMPOSITIONS AND METHODS OF USE

(71) Applicants: Board of Supervisors of Louisiana State University and Agricultural and Mechanical College, Baton Rouge, LA (US); University of New Orleans, New Orleans, LA (US)

(72) Inventors: Krzysztof Reiss, Metairie, LA (US); Branko Jursic, New Orleans, LA (US)

(21) Appl. No.: 17/421,980

(22) PCT Filed: Jan. 13, 2020
(86) PCT No.: PCT/US20/13317

§ 371 Date: Jul. 9, 2021

Related U.S. Application Data
(60) Provisional application No. 62/791,252, filed on Jan. 11, 2019.
(60) Provisional application No. 62/884,529, filed on Aug. 8, 2019.

(51) Int. Cl. A61K 31/165; A61P 35/00; A61K 31/495; A61K 31/5377; A61K 31/4965; A61K 31/47; A61K 31/473; A61K 31/426

(52) U.S. Cl. CPC A61K 31/165; A61P 35/00; A61K 31/495; A61K 31/5377; A61K 31/4965; A61K 31/47; A61K 31/473; A61K 31/426

(57) ABSTRACT

This invention is directed to anticancer compounds, pharmaceutical compositions comprising the same, methods of making anticancer compounds, and methods of treating cancer with compounds and pharmaceutical compositions.

This application claims priority from U.S. Provisional Application No. 62/791,252, filed Jan. 11, 2019, and U.S. Provisional Application No. 62/884,529, filed Aug. 8, 2019, the entire contents of each which are incorporated herein by reference.

### GOVERNMENT INTERESTS

This invention was made with government support under Grant No. P20-GM121288 awarded by the National Institutes of Health. The government has certain rights in the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed to anticancer compounds, pharmaceutical compositions comprising the same, methods of making anticancer compounds, and methods of treating cancer with compounds and pharmaceutical compositions.

### BACKGROUND OF THE INVENTION

Glial tumors account for nearly 50% of all adult primary intracranial neoplasms, among which GBM is the most aggressive and practically incurable. Glioblastoma multiforme (GBM) is a highly lethal brain tumor for which therapeutic options are limited. Rapidly growing and highly invasive GBM cells rely on both glycolysis and mitochondrial respiration to generate sufficient amounts of ATP and intermediate metabolites (anaplerosis). A large variety of different genetic and epigenetic modifications have been found in GBMs, among which p53 mutations, EGF receptor amplification, and PTEN mutations are most common. However, gene therapy, molecular and immunological approaches targeting these molecules and their pathways, as well as recently tested antibodies against immune checkpoint inhibitors have yet to produce improvements in patient outcomes.

### SUMMARY OF THE INVENTION

The present invention provides anticancer compounds, pharmaceutical compositions comprising anticancer compounds, methods of synthesizing anticancer compounds, and methods of using anticancer compounds to treat disease.

Aspects of the invention are directed towards an anticancer compound comprising the following formula:

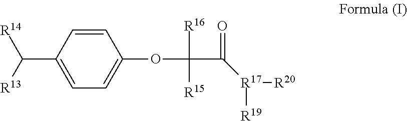

In an embodiment of Formula (I), R₁₃ can comprise substituted cycloalkyl, substituted aryl, substituted heterocycle, or substituted aromatic heterocycle; R₁₄ can comprise hydrogen or double bounded oxygen; R₁₅ comprises hydrogen, substituted alkyl, or substituted aryl; R₁₆ can comprise hydrogen, substituted alkyl, or substituted aryl; R₁₇ can comprise nitrogen or carbon; R₁₉ can comprise hydrogen, hydroxy, amino, substituted amino, alkyl, hydroxyalkyl, cycloalkyl, heterocycle, aryl, hydroxyaryl, aromatic heterocycle, or hydroxyaryl; R₂₀ can comprise hydrogen, hydroxy, amino, substituted amino, alkyl, hydroxyalkyl, cycloalkyl, heterocycle, aryl, hydroxyaryl, aromatic heterocycle, or hydroxyaryl; or any combination thereof.

In embodiments, an anticancer compound comprises the following formula:

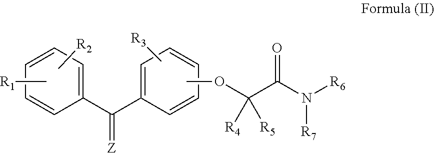

In an embodiment of Formula (II), R₁ can comprise hydrogen, halogen, alkyl, oxyalkyl, aryl, oxyaryl, halogen, cyano, nitro, carboxyl, or carboxyalkyl; R₂ can comprise hydrogen, halogen, alkyl, oxyalkyl, aryl, oxyaryl, halogen, cyano, nitro, carboxyl, or carboxyalkyl; R₃ can comprise hydrogen, halogen, alkyl, or aryl; R₄ can comprise hydrogen, substituted alkyl, or substituted aryl; R₅ can comprise hydrogen, substituted alkyl, or substituted aryl; R₆ can comprise hydrogen, hydroxy, amino, substituted amino, alkyl, hydroxyalkyl, cycloalkyl, heterocycle, aryl, hydroxyaryl, aromatic heterocycle, or hydroxyaryl; R₇ can comprise hydrogen, hydroxy, amino, substituted amino, alkyl, hydroxyalkyl, cycloalkyl, heterocycle, aryl, hydroxyaryl, aromatic heterocycle, or hydroxyaryl; or any combination thereof. In embodiments, R₆-R₇ can be part of cycle, such as but not limited to pyrrolidine, piperidine, morpholine, pyrrole, and their alkyl substituents. In embodiments, Z can comprise 2H, O, NNH₂, and NNHR, where R can be alkyl, aryl, acyl, aryloyl or any combination thereof.

In embodiments, the anticancer compound comprises one of the following structures:

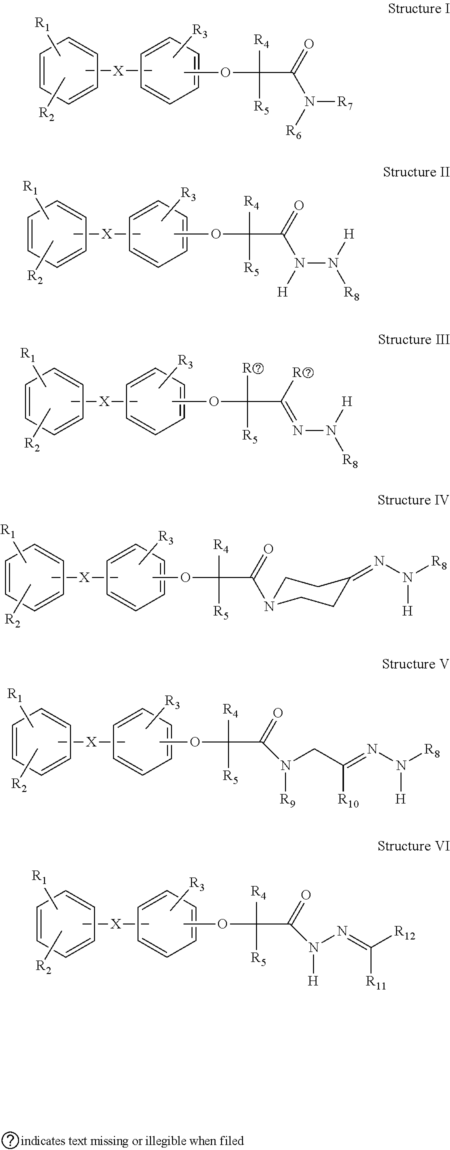

In embodiments, R₁, R₂, R₃ can comprise H, alkyl, O-alkyl, nitro, cyano, or halogen; R₄, R₅, R₆, R₉ can comprise H or alkyl; R₇ can comprise alkyl, substituted alkyl, hydroxy alkyl, aryl, substituted aryl, heteroaryl, substituted heteroaryl, hydroxy, or amino; R₅ can comprise aryl, substituted aryl, heteroaryl, substituted heteroaryl, CO-aryl, CO-heteroaryl, or CO-linker; R₁₀ can comprise alkyl, hydroxyalkyl, substituted aryl, or substituted heteroaryl; R₁, R₁₂ can comprise H, alkyl, aryl, heteroaryl, alkyl with linker, or aryl with linker; X can comprise CO, CH₂, O, CC, or CH═CH; Z can comprise O, or H₂; any combination thereof

For example, the anticancer compound comprises any one of the structures of Table 2.

In embodiments, the anticancer compound comprises any of the following structures:

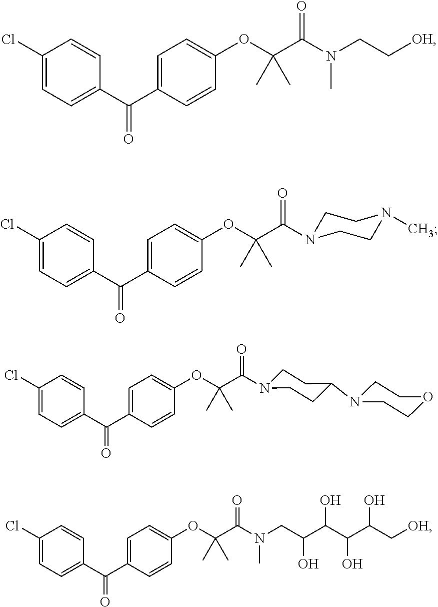 [SMILES: CN(CCO)C(=O)C(C)(C)Oc1ccc(C(=O)c2ccc(Cl)cc2)cc1 ; CN1CCN(C(=O)C(C)(C)Oc2ccc(C(=O)c3ccc(Cl)cc3)cc2)CC1 ; CC(C)(Oc1ccc(C(=O)c2ccc(Cl)cc2)cc1)C(=O)N1CCC(N2CCOCC2)CC1 ; CN(CC(O)C(O)C(O)C(O)CO)C(=O)C(C)(C)Oc1ccc(C(=O)c2ccc(Cl)cc2)cc1]

a pharmaceutically acceptable salt thereof, or a derivative thereof.

In embodiments, the anticancer compound comprises the following formula:

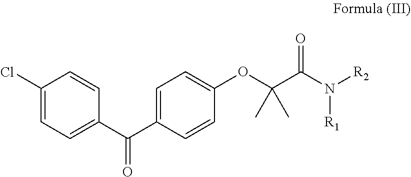

In embodiments, R₁ can be H, alkyl, hydroxyalkyl. In embodiments, R₂ can be H, alkyl, hydroxyalkyl, polyhydroxyalkyl, aminoalkyl, dialkylaminoalkyl. In embodiments, R₁-R₂ can be (CH₂)ₙ, (CH₂)ₙO(CH₂)ₘ, (CH₂)ₙCHOH(CH₂)ₘ, (CH₂)ₙNCH₃(CH₂)ₘ, where n and m are 1, 2, 3, 4, or 5.

For example, the anticancer compound comprises any one of the structures of Table 3.

In embodiments, the anticancer compound comprises any one of the structures of Table 3. For example, the anticancer compound comprises any of the following structures:

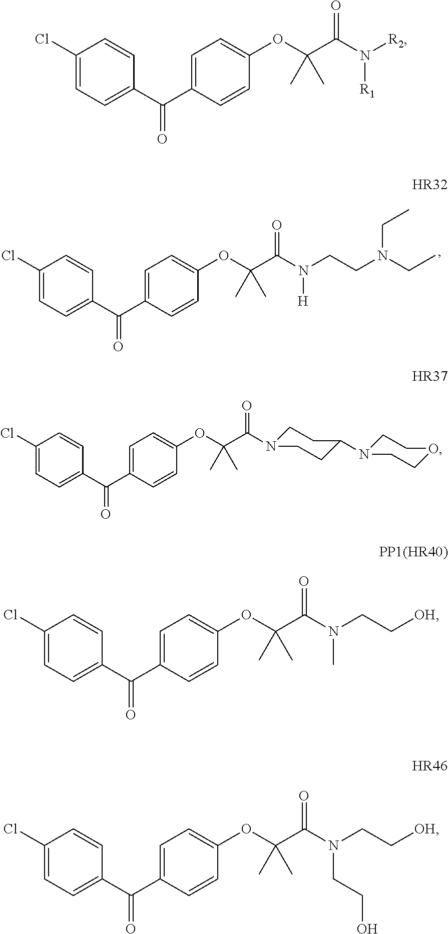

a pharmaceutically acceptable salt thereof, or a derivative thereof.

In embodiments, the anti cancer compound comprises the following formula:

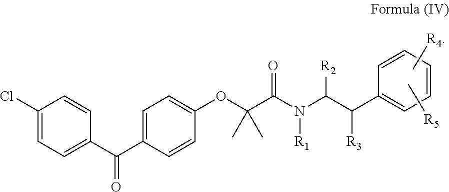

In embodiments, R₁ can be H or CH₃; R₂ can be OH, OCH₃, or any combination thereof; R₃ can be OH, OCH₃, or any combination thereof; R₄ can be OH, OCH₃, or any combination thereof; R₅ can be OH, OCH₃, or any combination thereof,

For example, the anti cancer compound can comprise any of the following structures:

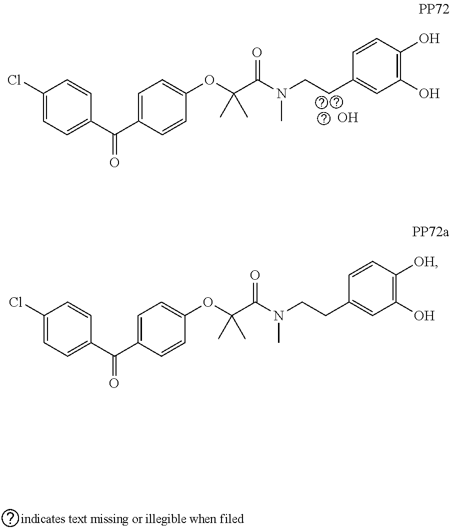

pharmaceutically acceptable salt thereof, or a derivative thereof.

In embodiments, the anti cancer compound comprises the following formula:

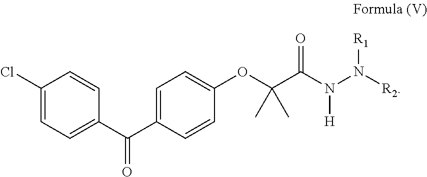

In embodiments, R₁ can be H, CH₃, or (CH₂)₅; R₂ can be H, CH₃, (CH₂)₅.

For example, the anti cancer compound comprises any of the following structures:

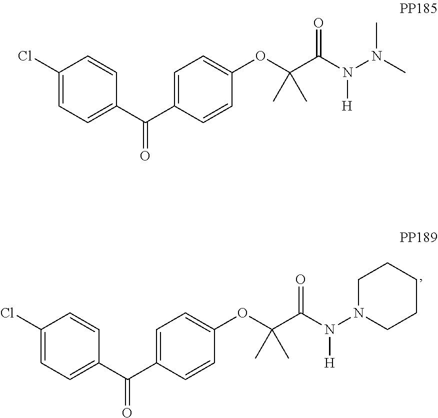 [SMILES: CN(C)NC(=O)C(C)(C)Oc1ccc(C(=O)c2ccc(Cl)cc2)cc1 ; CC(C)(Oc1ccc(C(=O)c2ccc(Cl)cc2)cc1)C(=O)NN1CCCCC1]

pharmaceutically acceptable salt thereof, or a derivative thereof.

In embodiments, the anti cancer compound can be the following formula:

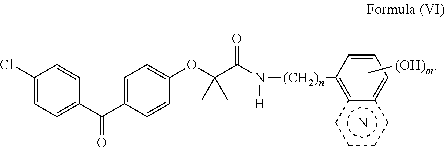

In embodiments, R₁ can be H or CH₃, R₂ can be OH and/or OCH₃, R₃ can be OH and/or OCH₃, R₄ can be OH and/or OCH₃, and/or R₅ can be OH and/or OCH₃.

For example, the anti cancer compound comprises any of the following structures:

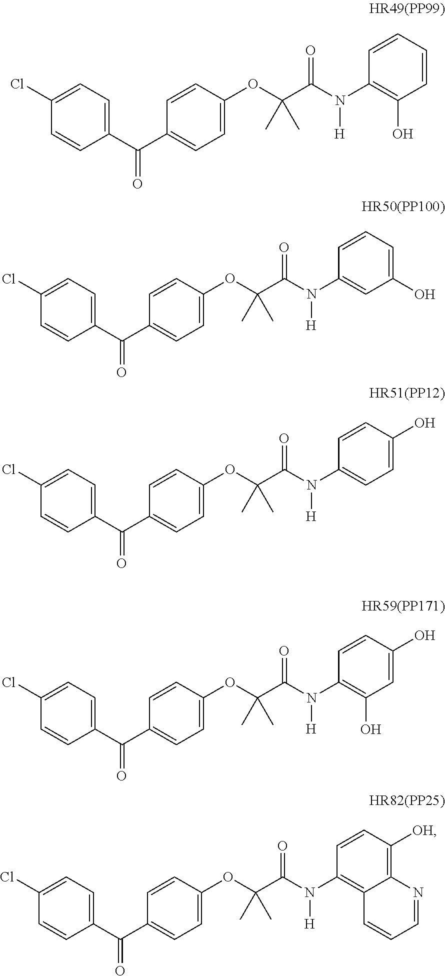 [SMILES: CC(C)(Oc1ccc(C(=O)c2ccc(Cl)cc2)cc1)C(=O)Nc1ccccc1O ; CC(C)(Oc1ccc(C(=O)c2ccc(Cl)cc2)cc1)C(=O)Nc1cccc(O)c1 ; CC(C)(Oc1ccc(C(=O)c2ccc(Cl)cc2)cc1)C(=O)Nc1ccc(O)cc1 ; CC(C)(Oc1ccc(C(=O)c2ccc(Cl)cc2)cc1)C(=O)Nc1ccc(O)cc1O ; CC(C)(Oc1ccc(C(=O)c2ccc(Cl)cc2)cc1)C(=O)Nc1ccc(O)c2ncccc12]

pharmaceutically acceptable salt thereof, or a derivative thereof.

In embodiments, the anti cancer compound can be the following formula:

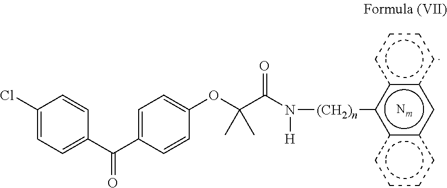

In embodiments, n can be 0, 1 or 2; and m can be 1 or 2.

For example, the anti cancer compound comprises any of the following structures:

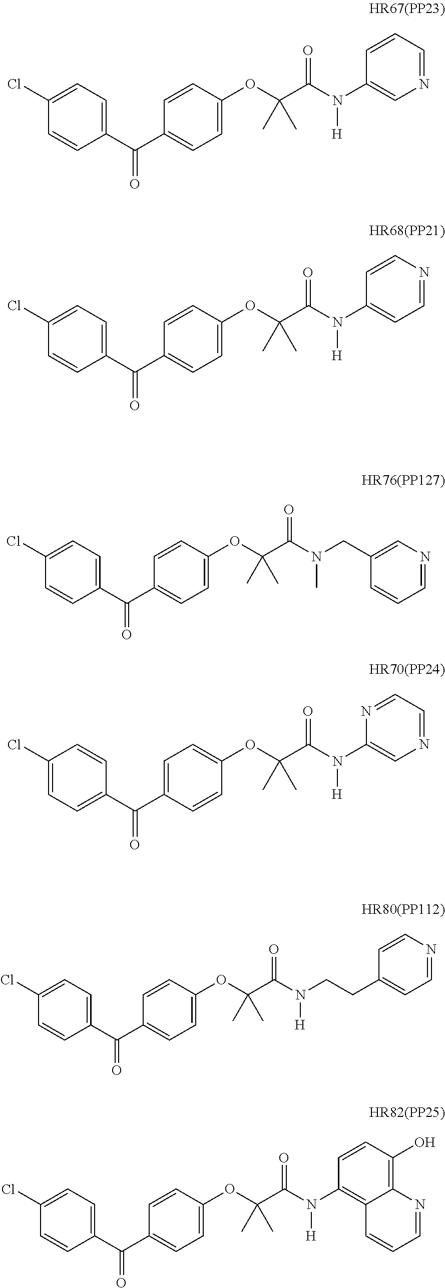 [SMILES: CC(C)(Oc1ccc(C(=O)c2ccc(Cl)cc2)cc1)C(=O)Nc1cccnc1 ; CC(C)(Oc1ccc(C(=O)c2ccc(Cl)cc2)cc1)C(=O)Nc1ccncc1 ; CN(Cc1cccnc1)C(=O)C(C)(C)Oc1ccc(C(=O)c2ccc(Cl)cc2)cc1 ; CC(C)(Oc1ccc(C(=O)c2ccc(Cl)cc2)cc1)C(=O)Nc1cnccn1 ; CC(C)(Oc1ccc(C(=O)c2ccc(Cl)cc2)cc1)C(=O)NCCc1ccncc1 ; CC(C)(Oc1ccc(C(=O)c2ccc(Cl)cc2)cc1)C(=O)Nc1ccc(O)c2ncccc12]

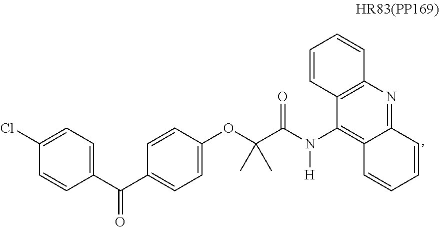 [SMILES: CC(C)(Oc1ccc(C(=O)c2ccc(Cl)cc2)cc1)C(=O)Nc1c2ccccc2nc2ccccc12]

pharmaceutically acceptable salt thereof, or a derivative thereof.

In embodiments, the anti cancer compound can be the following formula:

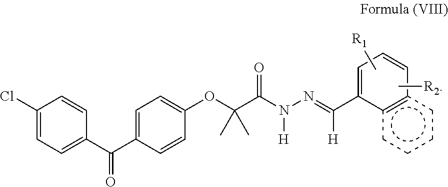

In embodiments, R₁ can be H, OH, Cl, Br, NO₂, and R₂ can be H, OH, Cl, Br, NO₂.

For example, the anti cancer compound can be any of the following structures:

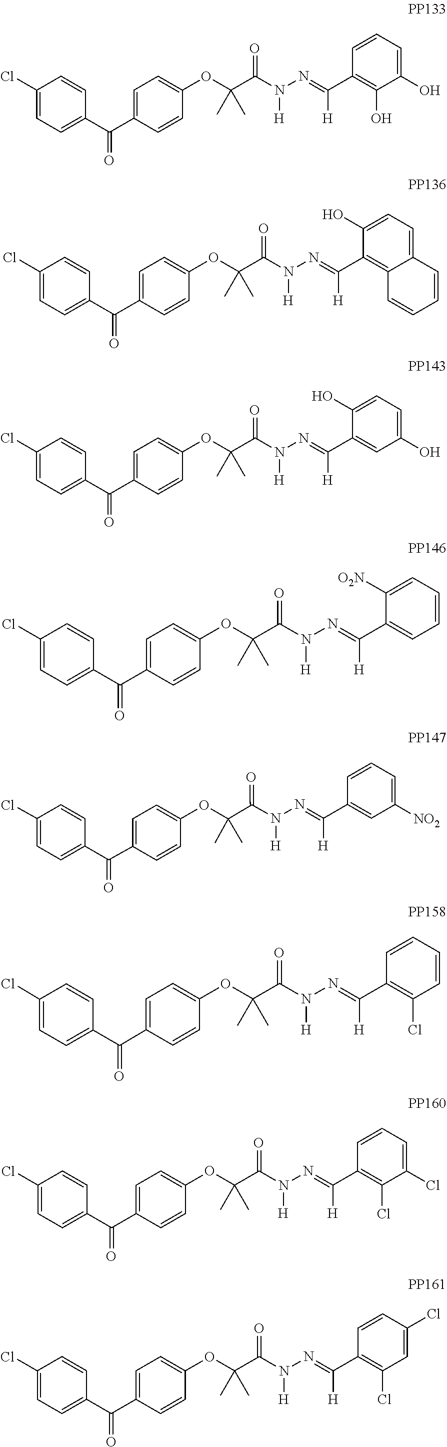

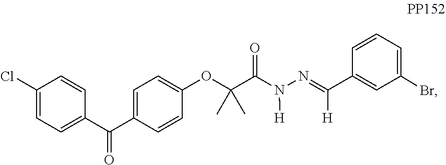 [SMILES: CC(C)(Oc1ccc(C(=O)c2ccc(Cl)cc2)cc1)C(=O)N/N=C/c1cccc(Br)c1]

pharmaceutically acceptable salt thereof, or a derivative thereof.

In embodiments, the anti cancer compound comprises the following formula:

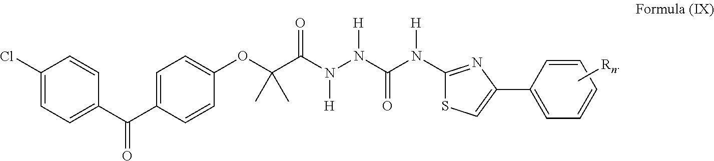

In embodiments, R can be H, Cl, or Br; and n can be 0, 1, 2, or 3.

For example, the anti cancer compound can be any of the following structures:

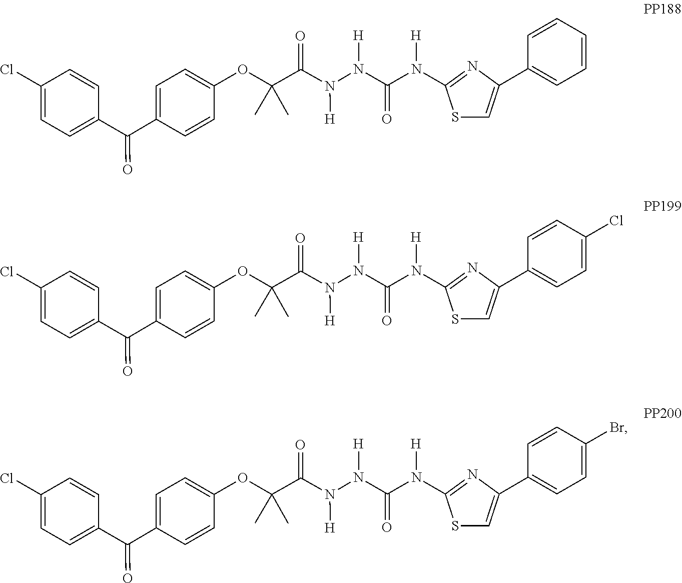 [SMILES: CC(C)(Oc1ccc(C(=O)c2ccc(Cl)cc2)cc1)C(=O)NNC(=O)Nc1nc(-c2ccccc2)cs1 ; CC(C)(Oc1ccc(C(=O)c2ccc(Cl)cc2)cc1)C(=O)NNC(=O)Nc1nc(-c2ccc(Cl)cc2)cs1 ; CC(C)(Oc1ccc(C(=O)c2ccc(Cl)cc2)cc1)C(=O)NNC(=O)Nc1nc(-c2ccc(Br)cc2)cs1]

a pharmaceutically acceptable salt thereof, or a derivative thereof.

In embodiments, the anti cancer compound comprises the following formula:

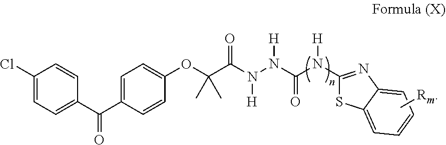

In embodiments, R can be H, Cl, or Br; n can be 0 or 1; m can be 1, 2, or 3.

For example, the anti cancer compound comprises any of the following structures:

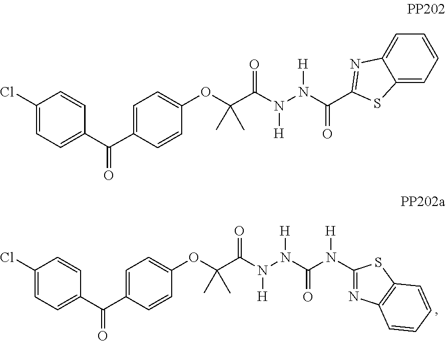 [SMILES: CC(C)(Oc1ccc(C(=O)c2ccc(Cl)cc2)cc1)C(=O)NNC(=O)c1nc2ccccc2s1 ; CC(C)(Oc1ccc(C(=O)c2ccc(Cl)cc2)cc1)C(=O)NNC(=O)Nc1nc2ccccc2s1]

pharmaceutically acceptable salt thereof, or a derivative thereof.

Further, aspects of the invention are directed towards a pharmaceutical composition comprising an anticancer compound described herein and a pharmaceutically acceptable carrier and/or pharmaceutically acceptable excipient. For example, the pharmaceutically acceptable carrier is albumin. For example, the pharmaceutical compositions can comprise an anticancer compound of Table 2, Table 3, or Example 4.

Still further, aspects of the invention are directed towards a method of synthesizing an anticancer compound as described herein. For example, the method comprises the schematic as indicated in FIG. 15.

Embodiments are directed towards a method of synthesizing 2-[4-(4-chlorobenzoyl)phenoxy]-N-(2-hydroxyethyl)-N,2-dimethylpropanamide (PP1), the method comprising: Dichloromethane (20 ml) suspension of fenofibric acid (318.75 mg; 1 mmol) and oxalyl chloride (0.25 mL; 380.1 mg; 3 mmol), and one drop od N,N-dimethylformamide was stirred at room temperature for 5 hours. Solvent was evaporated under reduced pressure. White solid residue was resolved in in dichloromethane (10 ml) and again evaporated to the solid residue. This solid residue was dissolved in dichloromethane (20 ml) and at room temperature with stirring dichloromethane (10 ml) solution of 2-(methylamino)ethanol (0.24 ml; 225 mg; 3 mmol) was gradually added. Reaction mixture was stirred at room temperature. Dichloromethane reaction mixture was washed with water (3×15 ml), 5% hydrochloric acid (3×15 ml), water (3×15 ml), 10% sodium carbonate (3×15 ml), and finally with water (3×15 ml) and dried over anhydrous sodium carbonate. Solvent was evaporated under reduced pressure to result in viscous pale-yellow liquid (390 mg). Product was crystalized from dichloromethane/hexane (30 ml; 1:4) at room temperature by slow solvent evaporation to ⅕ original volume and formed crystals were washed with ice cold hexane. Isolated yield 340 mg (90%).

Embodiments are also directed towards a method of synthesizing 2-[4-(4-chlorobenzoyl)phenoxy]-N-(2-hydroxyethyl)-N,2-dimethylpropanamide (PP1), the method comprising: Dichloromethane (100 ml) solution of fenofibric acid (637 mg; 2 mmol), 1-ethyl-3-(3-dimethylaminopropyl)carbodiimide, hydrochloride (EDC, 576; 3 mmol), and 2-(methylamino)ethanol (600 mg; 8 mmol) was stirred at room temperature overnight. Dichloromethane solution was washed with 5% hydrochloric acid (5×20 ml), water (5×20 ml), 10% sodium carbonate (5×20 ml), water (3×20 ml) and dried over anhydrous sodium carbonate. After solvent evaporation, oily residue was crystalized from dichloromethane/hexane (1:4) to give pure product (640 mg; 85% yield).

Embodiments are also directed towards a method of synthesizing 2-[4-(4-chlorobenzoyl)phenoxy]-2-methyl-1-(4-methylpiperazin-1-yl)propan-1-one (PP2), the method comprising: Dichloromethane (30 ml) of fenofibric chloride (1 mmol; prepared as described above for PP1 preparation) was slowly added in stirring water (5 ml) solution of sodium carbonate (216 mg; 2 mmol) with tetrahydrofuran (10 ml) and of 1-methylpiperazine (0.13 ml; 120 mg; 1.2 mmol). Resulting reaction mixture was stirred at room temperature for one hour. Additional water (30 ml) was added and organic layer was separated, washed with water (3×10 ml), 5% hydrochloric acid (3×10 ml), 10% sodium carbonate (3×10 ml), water and dried over anhydrous sodium carbonate. After evaporation oily residue was crystalized from dichloromethane hexane to give 350 mg (88%) of pure product.

Further, embodiments are directed towards a method of synthesizing 2-[4-(4-chlorobenzoyl)phenoxy]-N,2-dimethyl-N-[(2S,3R,4R,5R)-2,3,4,5,6-pentahydroxyhexyl]propenamide (PP3), the method comprising: Fenofibric acid chloride prepared from fenofibric acid (318.75 mg; 1 mmol) and oxalyl chloride (0.25 mL; 380.1 mg; 3 mmol) as described for preparation of PP1 was dissolved in dichloromethane (15 ml) and mixed with acetonitrile (20 ml) and water (10 ml) solution of N-methyl-D-glucamine (196 mg; 1 mmol) and sodium carbonate (212 mg; 2 mmol). Resulting mixture was stirred at room temperature for five minutes and solvent was evaporated under reduce pressure at room temperature. Resulting solid residue was mixed with dichloromethane (50 ml) and water (20 ml). Dichloromethane layer was separated, washed with 10% sodium carbonate (3×10 ml), water (3×10 ml) and dried over anhydrous sodium carbonate. After solvent was evaporated solid residue was washed with hexane (3×5 ml) and dried in vacuum under reduce pressure to give 350 mg (71%) of pure product.

Still further, embodiments are directed towards a method of synthesizing 2-[4-(4-chlorobenzoyl)phenoxy]-2-methyl-1-[4-(morpholin-4-yl)piperidin-1-yl]propan-1-one (PP4), the method comprising: Fenofibric acid chloride prepared from fenofibric acid (160 mg; 0.5 mmol) and oxalyl chloride (0.25 mL; 380.1 mg; 3 mmol) as described for preparation of PP1 was dissolved in dichloromethane (30 ml) and mixed with tetrahydrofuran (10 ml) solution of 4-morpholinopiperidine (100 mg; 0.6 mol), and water (10 ml) solution of sodium carbonate (106 mg; 1 mmol). Resulting mixture was stirred at room temperature for one hour. Water (20 ml) was added and organic layer was separated and extensively washed with 5% hydrochloric acid (5×20 ml), water (3×10 ml), 10% sodium carbonate (5×20 ml) and again with water (3×10 ml). After drying over anhydrous sodium carbonate solvent was evaporated to give pure product (200 mg; 85%) as oil that crystallized by standing at room temperature overnight. If necessary, the product can be further purified by crystallization from hexane or by silica gel chromatography with ethyl acetate-ethanol (5:1).

Also, embodiments are directed towards a method of synthesizing 4-(1-{2-[4-(4-chlorobenzoyl)phenoxy]-2-methylpropanoyl}piperidin-4-yl)morpholin-4-iumchloride (PP4HCl)—A mixture of concentrated hydrochloric acid (2 ml) and PP4 (94 mg; 0.2 mmol) was sonicated at room temperature for half an hour. Clear solution was evaporated to dryness at room temperature under reduced pressure. Residue was dissolved in dry ether (20 ml) and clear solution was left at room temperature for solvent to slowly evaporate. For white crystalline material was separated by filtration and washed with ice-cold hexane to give 80 mg (79%) of pure product.

Aspects of the invention are also directed towards a method of treating a subject afflicted with a disease, such as cancer, comprising administering to the subject a therapeutically effective amount of a pharmaceutical composition described herein, wherein the pharmaceutical composition comprises an anticancer compound described herein. Non-limiting examples of the cancer comprise a solid tumor or a liquid cancer; a brain cancer, such as glioma; a glioma, such as an astrocytoma, an ependymoma, or an oligodendrogliuma; glioblastoma; a blood cancer, such as leukemia, lymphoma, or hemangiosarcoma.

Further, aspects of the invention are directed towards a method of attenuating abnormal cell proliferation comprising administering to the subject in need thereof a therapeutically effective amount of the pharmaceutical composition of claim 12, wherein the composition attenuates abnormal cell proliferation. For example, the cell can comprise a cancer cell, such as a primary cancer cell, a brain cancer cell, or a blood cancer cell.

Still further, aspects of the invention are directed towards a method of inhibiting or delaying metastatic invasion of a cancer cell comprising administering to the subject in need thereof a therapeutically effective amount of the pharmaceutical composition described herein, wherein the composition inhibits or delays metastatic invasion of a cancer cell.

In embodiments, the pharmaceutical composition is administered orally to the subject.

Other objects and advantages of this invention will become readily apparent from the ensuing description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows (panel A) general structural motif of PP compounds. All starting materials were reagent grade purchased from Sigma-Aldrich or Ark Pharm. 1H-NMR spectra were recorded on Varian Mercury Plus 400 MHz instrument in CDCl3 or DMSO-d6, with the solvent chemical shifts as an internal standard. All computed molecular descriptors were generated by Chemaxon MarvinSketch version 18.8.0. (Panel B) Strategies for the preparation of PP compounds (see methods described herein for details).

TABLE 1 shows computed properties for piper derivatives.

FIG. 2 shows cytotoxic and cytostatic effects of PP compounds. Panel A: Human glioblastoma cells, LN-229 (ATCC CRL-2611), were cultured in 6-well plates at 2×105/well in DMEM containing 10% FBS. The percentage of cell death (panels A-F) and the total cell number (panels B-F) were calculated at time 0 (TO; 6 hrs after plating—plating efficiency), and at 24, 48, 72 and 96 hrs after treatments. The cells were treated with fenofibrate (FF) at 10, 25 and 50 μM (FF//10, FF/25, and FF/50), and with PP1, PP2, PP3, and PP4, at 5, 10, 25 and 50 μM. Control cultures were treated with corresponding volumes of dimethyl sulfoxide (DMSO), which was used as a solvent for both FF and PP compounds. At the end point of each experiments, the cells were harvested in a quantitative manner, treated with 0.4% trypan blue solution (Sigma) and counted using bright field hemocytometer. Data represent average values from 3 independent experiments in triplicate (n=9)+/−SD. * indicates PP1 or PP2 or PP3 or PP4 values significantly different from the corresponding time point values for DMSO treatment. In Panels B-F, both trypan blue-positive and trypan blue-negative cells were included in the calculation of cell number and % cell death is indicated at the bottom of the corresponding column. Data represent average values+/−SD, (n=3). * indicates PP1 or PP2 or PP3 or PP4 values significantly different from the DMSO value at 96 hrs.

FIG. 3 shows cytotoxic effects of PP compounds evaluated in three different glioblastoma cell lines. A: Human glioblastoma cell line, U-87MG (ATCC #HTB14); B: GBM12, which are patient-derived human glioblastoma cells (62); C: and mouse glioblastoma cell line GL-261-luc (PerkinElmer Inc.) (Panel C), were all cultured in 24-well plates at the initial density of 1×104 cells/cm2 in DMEM containing 10% FBS. The percentage of cell death was calculated at 96-hour time point for all experimental conditions. The cells were treated with PP1, at 5, 10, 25 and 50 μM, and control cultures were treated with corresponding volumes of dimethyl sulfoxide (DMSO; PP1 solvent). At the end of each experiments, the cells were harvested in a quantitative manner, treated with 0.4% trypan blue solution (Sigma) and counted using bright field hemocytometer. Data represent average values+/−SD from 3 independent experiments in triplicate (n=9). * indicates PP1 values significantly different from DMSO.

FIG. 4 panel A shows stability of PP1 and FF in human blood. Aliquots of 50 μM PP1 and FF in DMSO were added to 500 μl to heparinized human blood to a final concentration of 50 μM. Following incubation at 37° C. for the indicated time points, the samples were frozen at −80° C., and later prepared for HPLC-based detection of the compounds. Please note that FF is converted to FA in the presence of human blood. In the same condition, PP1 remains stable for a significantly longer time and FA is not produced. Data represent average values with standard deviation (n=3). panel B: Water solubility. FF and PP1, were added to water to a final concentration of 50 mM and sonicated in an ultrasonic bath (Branson 1510 Digital Heated Ultrasonic bath) for one hour at room temperature. The samples were then centrifuged for 10 minutes at 16000×g, and the supernatant filtered through 0.45 μm syringe filter. Water solubilized PP1 and FF were subsequently processed for HPLC analysis (see Methods). Data represent average values with standard deviation (n=3). panel C: Effects of PP compounds on PPAR responsive elements (PPRE). The PPAR transcriptional activity was determined in HepG2 cells by the JsTkpGL3 reporter plasmid, which contains a firefly luciferase gene driven by the PPRE, which consists of three copies of the J site from the apo-AII gene promoter. To normalize for efficiency of transfection the cells were additionally transfected with the pSV40-GLuc (New England Biolabs, Ipswich, Mass.) control plasmid. Twenty-four hours after transfection the cells were incubated with ciglitazone (30 μM), FF, PP1, PP2, PP3 and PP4 (all 25 μM) for an additional 24 hrs. The luciferase activity was detected with Dual-Luciferase reporter assay system (Promega, Madison, Wis.), and the resulting luminescence measured with Synergy 2 microplate reader (BioTek, Winooski, Vt.). Data represent average values with standard deviation (n=6).

FIG. 5 shows metabolic effects of PP compounds compared to FF. Metabolic responses of LN-229 human glioblastoma cells were evaluated with Extracellular Flux Analyzer XF24 (Seahorse Biosciences, North Billerica, Mass.). Prior each assay the cells were plated at 4×104 cells/well in 24-well plates in growth supporting media. At the time of measurement, growth media were replaced with serum-free XF medium (Seahorse) in cartridges equipped with oxygen-sensitive and pH-sensitive fluorescent probes. The oxygen consumption rate (OCR; indicative of mitochondrial respiration) and acidification rate (ECAR; indicative of glycolysis) were evaluated after injecting the following metabolic toxins: oligomycin, FCCP; rotenone; and antimycin A. Immediate metabolic effects were determined by comparing OCR and ECAR values in cells pre-incubated with DMSO (vehicle), or with PP1 and FF, all used at 25 μM. A: Representative experiment in which real OCR (pmol/min) and ECAR (mpH/min) values are shown. Other relevant metabolic parameters calculated from this experiment include ATP production, Spare Respiratory Capacity, basal OCR levels and proton leak. B: Average OCR and ECAR data (% change over basal OCR and ECAR values) from three independent experiments repeated 5 times (n=15). Data represent average values+/−SD. * indicates PP1 values significantly different from DMSO.

FIG. 6 shows PP1 tissue distribution and toxicity data. Panel A: PP1 accumulation in different tissues following oral administration of PP1 (25 mg/kg). C57BL/6 mice were treated daily for 14 days, and the levels of PP1 in the blood, liver, spleen, kidney, heart, brain and in brain tumors, were evaluated by HPLC, as previously described. Data represent average values with standard deviation (n=3). Panel B: Body weight of C57BL/6 mice treated with PP1. Mice were treated with the PP1 at doses ranging from 25 to 75 mg/kg/day administered by oral gavage. Following 7 and 14 days of daily drug administration both control (DMSO-treated) and experimental (PP1-treated) mice were weighted. Data represent average values with standard deviation (at least three mice per group were included). In Panels A and B, tumor free and tumor bearing mice were included for the body weight measurement. Panels C, D and E: Intracranial growth of GL-261-Luc cells. C57 black mice, 6-8 weeks of age were anesthetized with 4% isoflurane and secured in a stereotaxic head frame (Harvard Apparatus, Holliston Mass.). GL-261-luc cells (1×105 cells in 2 μl; PerkinElmer Inc.) were injected into the brain parenchyma (coordinates: 3 mm anterior to Bregma; 1.5 mm lateral to Sagital suture; 3 mm down from surface) through a burr hole in the skull using a 10 μl Hamilton syringe. Panel C: Mice with large intracranial tumors were selected using bioluminescence imaging with Xenogen IVIS 200 system. Tumor size is expressed as radiance (photons/s/cm2/sr) and was quantified with the Living Image 4.1 software according to the manufacturer's recommendations (Xenogen). Panels D and E: Pathological evaluation of intracranial tumors and tissues. Following 14 days of drug administration (control: DMSO administered daily by oral gavage; PP1 50 mg/kg administered daily by oral gavage) the animals were euthanized, and the following organs were collected: liver, kidneys, spleen, heart, intact brain and intracranial tumors. The tissues were formalin-fixed, paraffin-embedded, and the resulting thin section were stained with hematoxylin and eosin (H&E) for a routine pathological evaluation.

FIG. 7 shows cytotoxic effects of PP1. Human melanoma (Mel202); human prostate cancer (PC3) and triple negative breast cancer (MDAMB231BR) were cultured in 24-well plates at initial density of 1×104 cells/cm² in DMEM containing 10% FBS. The percentage of cell death was calculated at 96-hour time point for all experimental conditions. The cells were treated with DMSO (control) and with PP1 at 5 and 25 μM. At the end point of each experiment, the cells were harvested in a quantitative manner, cell viability assessed by Guava/ViaCount according to the manufacturer's recommendations. Data represent average values from 3 independent experiments in triplicate (n=6) with standard deviation.

FIG. 8 shows cytotoxic and cytostatic effects of PP compounds (PP5-PP18). Human glioblastoma cells, LN-229 (ATCC CRL-2611), were cultured in 24-well plates at initial density of 1×104 cells/cm² in DMEM containing 10% FBS. The percentage of cell death (Panel A) and cell number (Panel B) were calculated at 96-hour time point for all experimental conditions. The cells were treated with DMSO (control) and with PP5-PP18, all used at 25 μM. At the end of each experiment, the cells were harvested in a quantitative manner, cell viability/cell number were assessed by Guava/ViaCount according to the manufacturer's recommendations. Data represent average values from 3 independent experiments in triplicate (n=6) with standard deviation.

FIG. 9 shows embodiments of the invention.

FIG. 10 shows cytotoxic and cytostatic effects of embodiments of the invention. Effect*: CT=cytotoxic; CS=cytostatic; NA=not active.

FIG. 11 shows example linkers.

FIG. 12 shows NMR of embodiments of the invention.

FIG. 13 shows TOF MS ES⁺ of embodiments of the invention.

FIG. 14 shows computed solubility versus pH of embodiments of the invention.

FIG. 15 shows preparation schemes of embodiments of the invention.

FIG. 16 extensive accumulation of peroxisomes in cells treated with PP3, which resembled morphology of LN-229 cells treated with 50 μM FF. Red arrows indicated accumulation of peroxisomes (black dot-like structures) around the nucleus in PP3-treated cells but not in control (DMSO). (LN229 cells, DMEM 1 g/L glucose 10% FBS P/S).

FIG. 17 shows comparison between FF, FFA, and PP1 (modified FF²¹) structural and functional properties. The information regarding the compounds water solubility, stability in human blood, penetration of the blood brain barrier (BBB), and in vitro cytotoxicity were previously reported¹⁹⁻²¹.

FIG. 18 shows regions of the BPA skeleton selected for modification (circles). These regions were subsequently modified in search of the optimal anti-glioblastoma drug,

FIG. 19 shows schematic illustration of the procedure to develop esters of substituted phenoxyacetic esters and acids.

FIG. 20 shows schematic illustration of the procedure to substitute phenoxyacetamides.

FIG. 21 shows schematic illustration of the selective reduction of HR40 (PP1).

FIG. 22 shows schematic illustration of the procedure to develop ammonium salts of HR34.

FIG. 23 shows anti-glioblastoma activity and computed physical properties of fenofibrate (FF) and its simple amides. (A; top panel) Cell viability (MTT assay) following exposure to the indicated derivatives of FF (25 μM, for 72 hrs). (B; bottom panel) Computed physical properties of fenofibrate and its simple amides. CV=Cell viability (% of control) mean±SD at 25 μM; C log P=calculated partitioning; HBD=hydrogen bond donor at pH=7; HBA=hydrogen bond acceptor at pH=7; log BB=calculated blood-brain partition; MP=Molecular polarizability (Å³); PSA=Polar surface area (Å²); MPA=Minimal projection area (Å²); Log S=Aqueous solubility (mg/ml); MPO=Central nervous system multiparameter optimization (CNS MPO).

FIG. 24 shows the BPA derivatives with methylene and oxygen in region B. (A; top panel) Cell viability (MTT assay) following exposure to the indicated derivatives of FF (25 μM, for 72 hrs). (B; bottom panel) CV=Cell viability (% of control) mean±SD at 25 μM; C log P=calculated partitioning; HBD=hydrogen bond donor at pH=7; HBA=hydrogen bond acceptor at pH=7; C log BB=calculated blood-brain partition; PSA=Polar surface area (Å²); MPA=Minimal projection area (Å²); Log S=Aqueous solubility (mg/ml); MPO=Central nervous system multiparameter optimization (CNS MPO).

FIG. 25 shows electrostatic potential map for PP1, HR1, and HR4 generated by semi-empirical method PM3 as implemented in Spartan '18 version 1.1.0

FIG. 26 shows Fluoro- vs Chloro-benzylphenoxyacetamide. (A; top panel) Cell viability (MTT assay) following exposure to modified variants of PP1 in which chlorine atom was replaced (25 μM, for 72 hrs). (B; bottom panel) CV=Cell viability (% of control) mean±SD at 25 μM; C log P=calculated partitioning; HBD=hydrogen bond donor at pH=7; HBA=hydrogen bond acceptor at pH=7; C log BB=calculated blood-brain partition; PSA=Polar surface area (Å²); MPA=Minimal projection area (Å²); Log S=Aqueous solubility (mg/ml); MPO=Central nervous system multiparameter optimization (CNS MPO).

FIG. 27 shows comparison of electrostatic potential map of our fluoro compounds with electrostatic potential map of PP1

FIG. 28 shows drug candidates with unsubstituted alpha position of BPA. (A; top panel) Cell viability (MTT assay) following exposure to modified variants of PP1 with unsubstituted alpha position of BPA (25 μM, for 72 hrs). (B; bottom panel) CV=Cell viability (% of control) mean±SD at 25 μM; C log P=calculated partitioning; HBD=hydrogen bond donor at pH=7; HBA=hydrogen bond acceptor at pH=7; C log BB=calculated blood-brain partition; PSA=Polar surface area (Å²); MPA=Minimal projection area (Å²); Log S=Aqueous solubility (mg/ml); MPO=Central nervous system multiparameter optimization (CNS MPO).

FIG. 29 shows drug candidates with alpha monomethylated BPA. (A; top panel) Cell viability (MTT assay) following exposure to modified variants of PP1 with alpha monomethylated BPA (25 μM, for 72 hrs). (B; bottom panel) CV=Cell viability (% of control) mean±SD at 25 μM; C log P=calculated partitioning; HBD=hydrogen bond donor at pH=7; HBA=hydrogen bond acceptor at pH=7; C log BB=calculated blood-brain partition; PSA=Polar surface area (Å²); MPA=Minimal projection area (Å²); Log S=Aqueous solubility (mg/ml); MPO=Central nervous system multiparameter optimization (CNS MPO).

FIG. 30 shows comparison of the electrostatic potential maps di-, mono- and non-methylated drug candidate (HR13, HR18 and HR21) with PP1.

FIG. 31 shows drug candidates with a pH neutral amide moiety. (A; top panel) Cell viability (MTT assay) following exposure to modified variants of PP1 with pH neutral amide moiety (25 μM, for 72 hrs). (B; bottom panel) CV=Cell viability (% of control) mean±SD at 25 μM; C log P=calculated partitioning; HBD=hydrogen bond donor at pH=7; HBA=hydrogen bond acceptor at pH=7; C log BB=calculated blood-brain partition; PSA=Polar surface area (Å²); MPA=Minimal projection area (Å²); Log S=Aqueous solubility (mg/ml); MPO=Central nervous system multiparameter optimization (CNS MPO). H28 activities at M (38.31±3.50), 5 μM (65.90±2.82), and 1 μM (92.75±3.59).

FIG. 32 shows drug candidates with basic amide moiety—protonated and alkylated. (A; top panel) Cell viability (MTT assay) following exposure to modified variants of PP1 with basic amide moiety (25 μM, for 72 hrs). (B; bottom panel) CV=Cell viability (% of control) mean±SD at 25 μM; C log P=calculated partitioning; HBD=hydrogen bond donor at pH=7; HBA=hydrogen bond acceptor at pH=7; log BB=calculated blood-brain partition; PSA=Polar surface area (Å²); MPA=Minimal projection area (Å²); Log S=Aqueous solubility (mg/ml); MPO=Central nervous system multiparameter optimization (CNS MPO). H32 CV at 10 μM (41.49±7.94), 5 μM (77.76±7.24), and 1 μM (96.80±6.51); H35 CV at 10 μM (56.26±0.59), 5 μM (79.34±1.70), and 1 μM (93.64±2.08); H37 CV at 10 μM (47.02±1.23), 5 μM (75.02±1.42), and 1 μM (109.20±5.73); H38 CV at 10 M (52.00±1.86), 5 μM (74.89±1.79), and 1 μM (97.97±11.41)

FIG. 33 shows drug candidates with one or several hydroxy groups in the amide moiety. (A; top panel) Cell viability (MTT assay) following exposure to modified variants of PP1 with one or several hydroxy groups in the amide moiety (25 μM, for 72 hrs). (B; bottom panel) CV=Cell viability (% of control) mean±SD at 25 μM; C log P=calculated partitioning; HBD=hydrogen bond donor at pH=7; HBA=hydrogen bond acceptor at pH=7; log BB=calculated blood-brain partition; PSA=Polar surface area (Å²); MPA=Minimal projection area (Å²); Log S=Aqueous solubility (mg/ml); MPO=Central nervous system multiparameter optimization (CNS MPO). PP1 CV at 10 μM (43.67±1.88), 5 μM (70.03±2.04), and 1 μM (98.36±1.61); H46 CV at 10 μM (51.97±10.27), 5 μM (86.15±8.50), and 1 μM (103.00±2.98)

FIG. 34 shows low magnification phase contrast images of LN229 human glioblastoma cells treated with five selected drug candidates at 25 μM concentration. Control cells were treated with the equal volume of vehicle (DMSO). Images were taken 48 hours following the treatment.

FIG. 35 shows structures of synthesized ester and acids for preparation of BPA derivatives.

FIG. 36 shows NMR of embodiments of the invention.

FIG. 37 shows NMR of embodiments of the invention.

FIG. 38 shows comparison between FF, FFA, and PP1 structural and functional (anti-cancer) properties.

FIG. 39 shows phenol region of BPA skeleton selected for modification (circle) in search of the optimal anti-glioblastoma drug.

FIG. 40 shows schematic illustration of the preparation procedure for preparation of hydroxylated phenyl and naphthyl derivatives of BPA.

FIG. 41 shows drug candidates with hydroxy substituted phenylamide moiety. Panel A: Cell viability (MTT assay) following exposure to modified variants of HR48 with one several hydroxy groups in the phenylamide moiety (25 μM, for 72 hrs). Panel B: CV=Cell viability (% of control) mean±SD at 25 μM; C log P=calculated partitioning; HBD=hydrogen bond donor at pH=7; HBA=hydrogen bond acceptor at pH=7; log BB=calculated blood-brain partition; PSA=Polar surface area (Å²); MPA=Minimal projection area (Å²); Log S=Aqueous solubility (mg/ml); MPO=Central nervous system multiparameter optimization (CNS MPO). Panel C: Electrostatic potential map for H48-HR51. Panel D: Computed HOMO orbitals contribution with their energies generated by semi-empirical method PM3 as implemented in Spartan '18 version 1.1.0

FIG. 42 shows Drug candidates with substituted 2-hydroxyphenylamide moiety. Panel A: Cell viability (MTT assay) following exposure to modified variants of HR48 with ortho hydroxy and ether methyl, chloro, or carboxy group in the phenylamide moiety (25 μM, for 72 hrs). Panel B: CV=Cell viability (% of control) mean SD at 25 μM; C log P=calculated partitioning; HBD=hydrogen bond donor at pH=7; HBA=hydrogen bond acceptor at pH=7; log BB=calculated blood-brain partition; PSA=Polar surface area (Å²); MPA=Minimal projection area (Å²); Log S=Aqueous solubility (mg/ml); MPO=Central nervous system multiparameter optimization (CNS MPO).). Panel C: Electrostatic potential map for H52-HR55. Panel D: Computed HOMO orbitals contribution with their energies generated by semi-empirical method PM3 as implemented in Spartan '18 version 1.1.0

FIG. 43 shows drug candidates with nitro-hydroxy and two hydroxy substituted phenylamide moiety. Panel A: Cell viability (MTT assay) following exposure to modified variants of HR48 with one hydroxy and one nitro group or with two hydroxy groups in the phenylamide moiety (25 μM, for 72 hrs). Panel B: CV=Cell viability (% of control) mean±SD at 25 μM; C log P=calculated partitioning; HBD=hydrogen bond donor at pH=7; HBA=hydrogen bond acceptor at pH=7; log BB=calculated blood-brain partition; PSA=Polar surface area (Å²); MPA=Minimal projection area (Å²); log S=Aqueous solubility (mg/ml); MPO=Central nervous system multiparameter optimization (CNS MPO). Panel C: Electrostatic potential map for H56-HR59. Panel D: Computed HOMO orbitals contribution with their energies generated by semi-empirical method PM3 as implemented in Spartan '18 version 1.1.0

FIG. 44 shows Drug candidates with hydroxy substituted naphthylamide moiety. Panel A: Cell viability (MTT assay) following exposure to modified variants of 1- and 2-naphthylamide of HR60 and HR64 with one hydroxy group in the naphthylamide moiety (25 μM, for 72 hrs). Panel B: CV=Cell viability (% of control) mean±SD at 25 μM; C log P=calculated partitioning; HBD=hydrogen bond donor at pH=7; HBA=hydrogen bond acceptor at pH=7; log BB=calculated blood-brain partition; PSA=Polar surface area (Å²); MPA=Minimal projection area (Å²); log S=Aqueous solubility (mg/ml); MPO=Central nervous system multiparameter optimization (CNS MPO). Panel C: Electrostatic potential map for H60-HR65. Panel D: Computed HOMO orbitals contribution with their energies generated by semi-empirical method PM3 as implemented in Spartan '18 version 1.1.0

FIG. 45 shows compiled three variation of computing log BB and estimated MPO-CNS values for HR48-HR65. log P computed partition; PSA=Polar surface area (Å²); D+A=Number hydrogen bond donor and acceptors; MPO=Central nervous system multiparameter optimization (CNS MPO); M1 Log BB=0.5159×log P −0.0277×PSA-0.3462 when expected is 0.3 [Vilar, S.; Chakrabarti, M.; Costanzi, S. “Prediction of passive blood-brain partitioning: Straightforward and effective classification models based on silico derived physicochemical descriptors” Journal of Molecular Graphics and Modelling 2010, 28, 899-903.]; M2 log BB=0.152 log P−0.0148PSA+0.139. (Clark's model); M3 log BB=0.155×log P−0.01×PSA+0.164. (Rishton's model); M4 log BB=0.2289×log P−0.0326×PSA−0.5671×(D+A)+2.3420 when expected log BB-1 [Vilar, S.; Chakrabarti, M.; Costanzi, S. “Prediction of passive blood-brain partitioning: Straightforward and effective classification models based on silico derived physicochemical descriptors” Journal of Molecular Graphics and Modelling 2010, 28, 899-903.]

FIG. 46 shows low magnification phase contrast images of LN229 human glioblastoma cells treated with four selected drug candidates at 25 μM. Control cells were treated with the equal volume of vehicle (DMSO). Images were taken 72 hours following the treatment.

FIG. 47 shows IC50 graph two anti-glioblastoma drug candidates.

FIG. 48 shows Drug candidates with unsubstituted phenol moiety. Panel A: Cell viability (MTT assay) following exposure to modified variants of PP1 with one or several hydroxy groups in the amide moiety (25 μM, for 72 hrs). Panel B: CV=Cell viability (% of control) mean±SD at 25 μM; C log P=calculated partitioning; HBD=hydrogen bond donor at pH=7; HBA=hydrogen bond acceptor at pH=7; log BB=calculated blood-brain partition; PSA=Polar surface area (Å²); MPA=Minimal projection area (Å²); Log S=Aqueous solubility (mg/ml); MPO=Central nervous system multiparameter optimization (CNS MPO).

FIG. 49 shows drug candidates with unsubstituted phenol moiety. Panel A: Cell viability (MTT assay) following exposure to modified variants of PP1 with one or several hydroxy groups in the amide moiety (25 μM, for 72 hrs). Panel B: CV=Cell viability (% of control) mean±SD at 25 μM; C log P=calculated partitioning; HBD=hydrogen bond donor at pH=7; HBA=hydrogen bond acceptor at pH=7; log BB=calculated blood-brain partition; PSA=Polar surface area (Å²); MPA=Minimal projection area (Å²); Log S=Aqueous solubility (mg/ml); MPO=Central nervous system multiparameter optimization (CNS MPO).

FIG. 50 shows drug candidates with unsubstituted phenol moiety. Panel A: Cell viability (MTT assay) following exposure to modified variants of PP1 with one or several hydroxy groups in the amide moiety (25 μM, for 72 hrs). Panel B: CV=Cell viability (% of control) mean±SD at 25 μM; C log P=calculated partitioning; HBD=hydrogen bond donor at pH=7; HBA=hydrogen bond acceptor at pH=7; log BB=calculated blood-brain partition; PSA=Polar surface area (Å²); MPA=Minimal projection area (Å²); Log S=Aqueous solubility (mg/ml); MPO=Central nervous system multiparameter optimization (CNS MPO).

FIG. 51 shows drug candidates with unsubstituted phenol moiety. Panel A: Cell viability (MTT assay) following exposure to modified variants of PP1 with one or several hydroxy groups in the amide moiety (25 μM, for 72 hrs). Panel B: CV=Cell viability (% of control) mean±SD at 25 μM; C log P=calculated partitioning; HBD=hydrogen bond donor at pH=7; HBA=hydrogen bond acceptor at pH=7; log BB=calculated blood-brain partition; PSA=Polar surface area (Å²); MPA=Minimal projection area (Å²); Log S=Aqueous solubility (mg/ml); MPO=Central nervous system multiparameter optimization (CNS MPO).

FIG. 52 shows NMR of embodiments of the invention.

FIG. 53 shows structures of hydroxy only N-phenyl BPA derivatives (i.e., Scheme 1).

FIG. 54 shows embodiments of the invention. HLB=hydrophilic-lipophilic balance; log S=water solubility; PL=polarizability (Å3); Minimal Projection Area (Å2); MSA=molecular surface area; RF=Refractivity; HBA=hydrogen bond acceptors; Log BB=blood-brain distribution; C log P=partitioning; C log D=distribution at pKa; TPSA=polar surface area; HBD=hydrogen bond donors; CNS MPO=score for CNS penetration.

FIG. 55 shows chloro hydroxyphenyl BPA derivatives (CP derivatives) (i.e., Scheme 2).

FIG. 56 shows embodiments of the invention. HLB=hydrophilic-lipophilic balance; log S=water solubility; PL=polarizability (Å3); Minimal Projection Area (Å2); MSA=molecular surface area; RF=Refractivity; HBA=hydrogen bond acceptors; Log BB=blood-brain distribution; C log P=partitioning; C log D=distribution at pKa; TPSA=polar surface area; HBD=hydrogen bond donors; CNS MPO=score for CNS penetration.

FIG. 57 shows nitro hydroxyphenyl BPA derivatives (NP derivatives) (i.e., Scheme 3).

FIG. 58 shows embodiments of the invention. HLB=hydrophilic-lipophilic balance; log S=water solubility; PL=polarizability (Å3); Minimal Projection Area (Å2); MSA=molecular surface area; RF=Refractivity; HBA=hydrogen bond acceptors; Log BB=blood-brain distribution; C log P=partitioning; C log D=distribution at pKa; TPSA=polar surface area; HBD=hydrogen bond donors; CNS MPO=score for CNS penetration.

FIG. 59 shows methyl hydroxyphenyl BPA derivatives (MP derivatives) (i.e., Scheme 4).

FIG. 60 shows embodiments of the invention. HLB=hydrophilic-lipophilic balance; log S=water solubility; PL=polarizability (Å3); Minimal Projection Area (Å2); MSA=molecular surface area; RF=Refractivity; HBA=hydrogen bond acceptors; Log BB=blood-brain distribution; C log P=partitioning; C log D=distribution at pKa; TPSA=polar surface area; HBD=hydrogen bond donors; CNS MPO=score for CNS penetration.

FIG. 61 shows carboxylic acid hydroxyphenyl BPA derivatives (CO derivatives) (i.e., Scheme 5).

FIG. 62 shows embodiments of the invention. HLB=hydrophilic-lipophilic balance; log S=water solubility; PL=polarizability (Å3); Minimal Projection Area (Å2); MSA=molecular surface area; RF=Refractivity; HBA=hydrogen bond acceptors; Log BB=blood-brain distribution; C log P=partitioning; C log D=distribution at pKa; TPSA=polar surface area; HBD=hydrogen bond donors; CNS MPO=score for CNS penetration.

FIG. 63 shows hydroxynaphthalene BPA derivatives (NA derivatives) (i.e., Scheme 6).

FIG. 64 shows HLB=hydrophilic-lipophilic balance; log S=water solubility; PL=polarizability (Å3); Minimal Projection Area (Å2); MSA=molecular surface area; RF=Refractivity; HBA=hydrogen bond acceptors; Log BB=blood-brain distribution; C log P=partitioning; C log D=distribution at pKa; TPSA=polar surface area; HBD=hydrogen bond donors; CNS MPO=score for CNS penetration.

FIG. 65 shows known drugs used for C log BB calibration.

FIG. 67 shows chemical compounds.

FIG. 68 shows chemical compounds and characteristics thereof.

FIG. 69 shows nine compounds with excellent anti-glioblastoma activity (LN229) at 25 μM.

FIG. 70 shows embodiments of the invention.

FIG. 71 shows computed Log BB values for previously reported active compounds.

FIG. 72 shows IC50 values for HR68 and HR69.

FIG. 73 shows concentration dependent activity for HR67(PP23) and HR68(PP21).

### DETAILED DESCRIPTION OF THE INVENTION

Anticancer effects of the lipid-lowering drug, fenofibrate (FF) have been described in the literature for a quite some time, however, FF has not been used as a direct anticancer therapy. FF in its unprocessed form accumulates in mitochondria, inhibits mitochondrial respiration, triggering a severe energy deficit and extensive glioblastoma cell death. However, FF does not cross blood brain barrier, and is quickly processed by blood and tissue esterases to form PPARα agonist, fenofibric acid (FA). In comparison to unprocessed FF, FA is much less effective in triggering cancer cell death.

The present invention provides anticancer compounds comprising chemical modifications to improve stability, water solubility, tissue penetration, and ultimately, anti-glioblastoma efficacy relative to FF. As described herein, data shows that the embodiments of the invention have improved cytotoxicity against glioblastoma cells in vitro in comparison to FF, and block mitochondrial respiration similarly to FF. In addition, embodiments of the invention are significantly more stable than FF when exposed to human blood, and have much better solubility in water when compared to FF. Mice orally administered embodiments of the invention demonstrated accumulation of the compound at therapeutically relevant concentrations in several tissues, including intracranial glioblastoma tumors, and survived the treatment without any major signs of distress. Importantly, treatment of mice bearing large intracranial glioblastoma tumors with embodiments of the invention resulted in extensive areas of necrosis within the tumor mass, thus demonstrating anti-glioblastoma efficacy of such novel metabolically active compounds. The anticancer effect of embodiments described herein is attributed to targeting cancer cell energy metabolism, which is very different in comparison to normal cells (see, for example, the Warburg effect).

### Abbreviations and Definitions

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Compounds of the Invention

Aspects of the invention are directed towards anticancer compounds comprising chemical compounds with improved stability, water solubility, tissue penetration, and ultimately, anti-glioblastoma efficacy relative to fenofibrate (FF).

An “anticancer compound” can refer to a compound effective in the treatment of cancer. This includes compounds, which kill the tumor cells and/or reduce the size of the tumor and/or reduce the growth and/or spreading or migration of the tumor or cancer cells. The term also encompasses traditional chemotherapeutic drugs and cytotoxic drugs. Other exemplary anti-cancer compounds include, e.g., neomycin, podophyl-lotoxin(s), TNF-alpha, calcium ionophores, calcium-flux inducing compounds, anti-tubulin drugs, colchicine, taxol, vinblastine, vincristine, vindescine, and combretastatin.

In one aspect of the invention, the anticancer compound is a compound of Formula I.

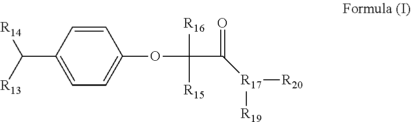

In an embodiment of Formula (I), R₁₃ can comprise substituted cycloalkyl, substituted aryl, substituted heterocycle, or substituted aromatic heterocycle; R₁₄ can comprise hydrogen or double bounded oxygen; R₁₅ can comprise hydrogen, substituted alkyl, or substituted aryl; R₁₆ comprises hydrogen, substituted alkyl, or substituted aryl; R₁₇ can comprise nitrogen or carbon; R₁₉ comprises hydrogen, hydroxy, amino, substituted amino, alkyl, hydroxyalkyl, cycloalkyl, heterocycle, aryl, hydroxyaryl, aromatic heterocycle, or hydroxyaryl; R₂₀ can comprise hydrogen, hydroxy, amino, substituted amino, alkyl, hydroxyalkyl, cycloalkyl, heterocycle, aryl, hydroxyaryl, aromatic heterocycle, or hydroxyaryl; or any combination thereof.

In another aspect of the invention, the anticancer compound is a compound of Formula II.

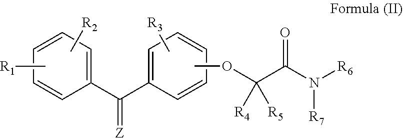

In an embodiment of Formula (II), R₁ can comprise hydrogen, halogen, alkyl, oxyalkyl, aryl, oxyaryl, halogen, cyano, nitro, carboxyl, or carboxyalkyl; R₂ can comprise hydrogen, halogen, alkyl, oxyalkyl, aryl, oxyaryl, halogen, cyano, nitro, carboxyl, or carboxyalkyl; R₃ can comprise hydrogen, halogen, alkyl, or aryl; R₄ can comprise hydrogen, substituted alkyl, or substituted aryl; R₅ can comprise hydrogen, substituted alkyl, or substituted aryl; R₆ can comprise hydrogen, hydroxy, amino, substituted amino, alkyl, hydroxyalkyl, cycloalkyl, heterocycle, aryl, hydroxyaryl, aromatic heterocycle, or hydroxyaryl; R₇ can comprise hydrogen, hydroxy, amino, substituted amino, alkyl, hydroxyalkyl, cycloalkyl, heterocycle, aryl, hydroxyaryl, aromatic heterocycle, or hydroxyaryl; or any combination thereof. In embodiments, R₆-R₇ can be part of cycle, such as but not limited to pyrrolidine, piperidine, morpholine, pyrrole, and their alkyl substituents. In embodiments, Z can comprise 2H, O, NNH₂, and NNHR, where R can be alkyl, aryl, acyl, aryloyl or any combination thereof.

In still another aspect of the invention, the anticancer compound is a compound of Structure (I):

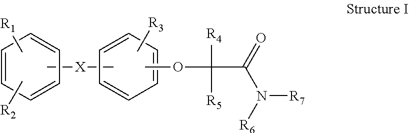

In another aspect of the invention, the anticancer compound is a compound of Structure (II):

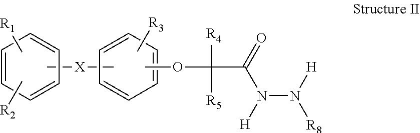

In another aspect of the invention, the anticancer compound is a compound of Structure (III):

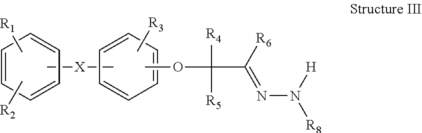

In another aspect of the invention, the anticancer compound is a compound of Structure (IV):

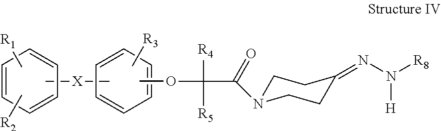

In another aspect of the invention, the anticancer compound is a compound of Structure (V):

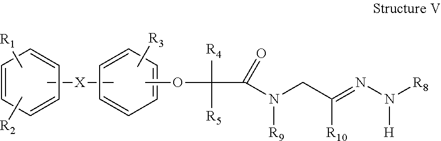

In another aspect of the invention, the anticancer compound is a compound of Structure (VI):

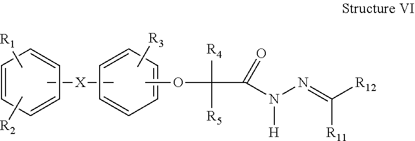

In embodiments described herein R₁, R₂, R₃ can be H, alkyl, O-alkyl, nitro, cyano, halogen; R₄, R₅, R₆, R₉ can be H or alkyl; R₇ can be alkyl, substituted alkyl, hydroxy alkyl, aryl, substituted aryl, heteroaryl, substituted heteroaryl, hydroxy, amino; R₅ can be aryl, substituted aryl, heteroaryl, substituted heteroaryl, CO-aryl, CO-heteroaryl, and CO-linker; R₁₀ can be alkyl, hydroxyalkyl, substituted aryl, substituted heteroaryl; R₁, R₁₂ can be H, alkyl, aryl, heteroaryl, alkyl with linker, aryl with linker; X can be CH₂, CHCH, CC, CO, or O; Z can be O, H₂, or any combination thereof.

Non-limiting examples of compounds of the invention comprise those compounds of Table 2.

Table 2 refers to compounds of the invention.

| Com- | | | |
|---|---|---|---|
| pound | Structure | Formula | MW |
| PP1 | 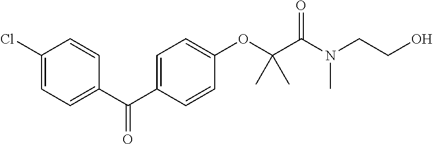 [SMILES: CN(CCO)C(=O)C(C)(C)Oc1ccc(C(=O)c2ccc(Cl)cc2)cc1] | C₂₀H₂₂ClNO₄ | 375.85 |
| PP2 | 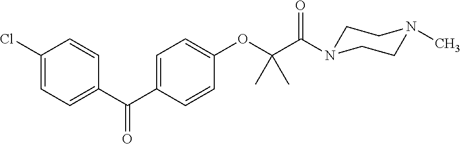 [SMILES: CN1CCN(C(=O)C(C)(C)Oc2ccc(C(=O)c3ccc(Cl)cc3)cc2)CC1] | C₂₂H₂₅ClN₂O₃ | 400.90 |
| PP2HCl | 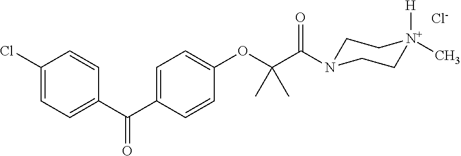 [SMILES: C[NH+]1CCN(C(=O)C(C)(C)Oc2ccc(C(=O)c3ccc(Cl)cc3)cc2)CC1 ; [Cl-]] | C₂₂H₂₆Cl₂N₂O₃ | 437.36 |
| PP2MeI | 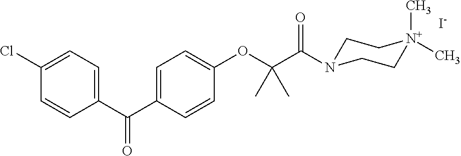 [SMILES: CC(C)(Oc1ccc(C(=O)c2ccc(Cl)cc2)cc1)C(=O)N1CC[N+](C)(C)CC1 ; [I-]] | C₂₃H₂₈ClIN₂O₃ | 542.84 |
| PP3 | 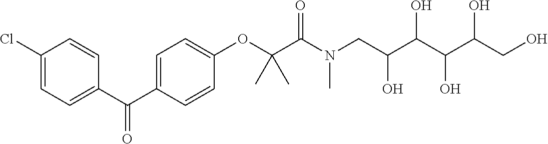 [SMILES: CN(CC(O)C(O)C(O)C(O)CO)C(=O)C(C)(C)Oc1ccc(C(=O)c2ccc(Cl)cc2)cc1] | C₂₄H₃₀ClNO₈ | 495.95 |
| PP4 | 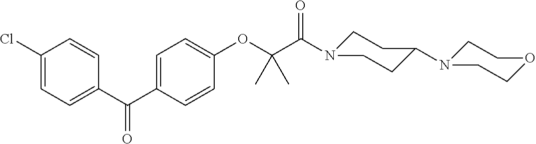 [SMILES: CC(C)(Oc1ccc(C(=O)c2ccc(Cl)cc2)cc1)C(=O)N1CCC(N2CCOCC2)CC1] | C₂₆H₃₁ClN₂O₄ | 470.99 |
| PP4HCl | 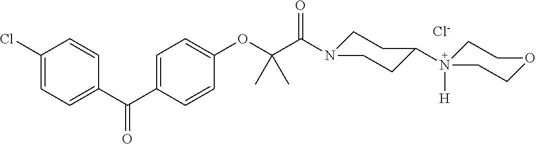 | C₂₆H₃₂Cl₂N₂O₄ | 507.45 |
| PP5 | 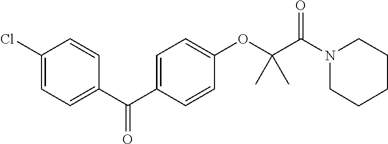 [SMILES: CC(C)(Oc1ccc(C(=O)c2ccc(Cl)cc2)cc1)C(=O)N1CCCCC1] | C₂₂H₂₄ClNO₃ | 385.88 |
| PP6 | 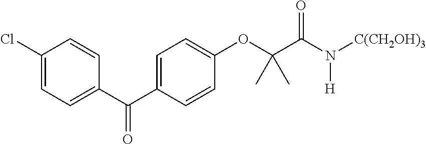 | C₂₁H₂₄ClNO₆ | 421.87 |
| PP7 | 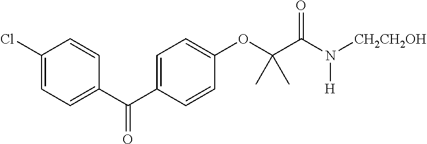 | C₁₉H₂₀ClNO₄ | 361.82 |
| PP8 | 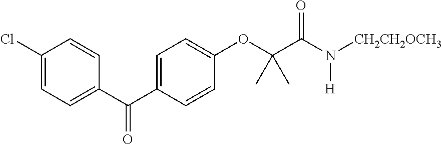 | C₂₀H₂₂ClNO₄ | 375.85 |
| PP9 | 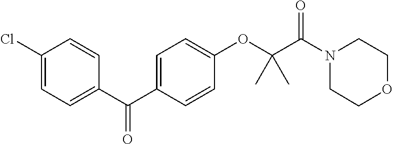 [SMILES: CC(C)(Oc1ccc(C(=O)c2ccc(Cl)cc2)cc1)C(=O)N1CCOCC1] | C₂₁H₂₂ClNO₄ | 387.86 |
| PP10 | 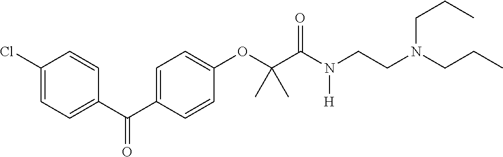 [SMILES: CCCN(CCC)CCNC(=O)C(C)(C)Oc1ccc(C(=O)c2ccc(Cl)cc2)cc1] | C₂₅H₃₃ClN₂O₃ | 444.99 |
| PP11 | 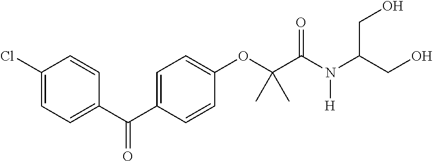 [SMILES: CC(C)(Oc1ccc(C(=O)c2ccc(Cl)cc2)cc1)C(=O)NC(CO)CO] | C₂₀H₂₂ClNO₅ | 391.85 |
| PP12 | 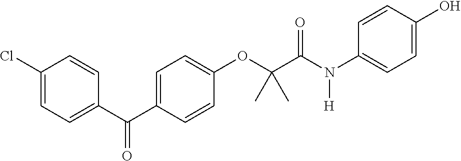 [SMILES: CC(C)(Oc1ccc(C(=O)c2ccc(Cl)cc2)cc1)C(=O)Nc1ccc(O)cc1] | C₂₃H₂₀ClNO₄ | 409.86 |
| PP13 | 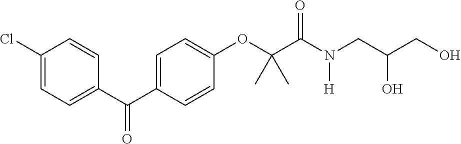 [SMILES: CC(C)(Oc1ccc(C(=O)c2ccc(Cl)cc2)cc1)C(=O)NCC(O)CO] | C₂₀H₂₂ClNO₅ | 391.85 |
| PP14 | 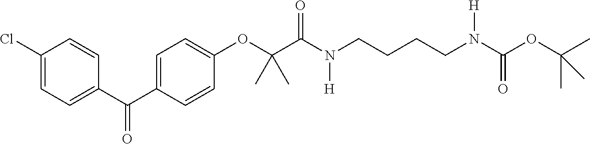 [SMILES: CC(C)(C)OC(=O)NCCCCNC(=O)C(C)(C)Oc1ccc(C(=O)c2ccc(Cl)cc2)cc1] | C₂₆H₃₃ClN₂O₅ | 489.00 |
| PP15 | 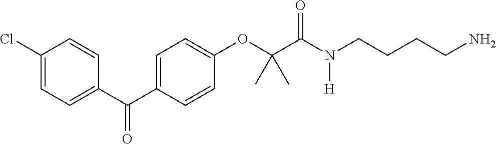 [SMILES: CC(C)(Oc1ccc(C(=O)c2ccc(Cl)cc2)cc1)C(=O)NCCCCN] | C₂₁H₂₅ClN₂O₃ | 388.89 |
| PP16 | 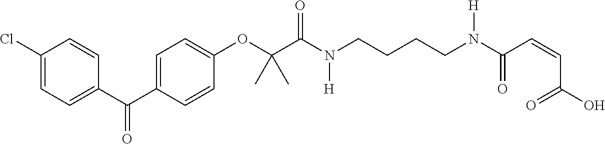 [SMILES: CC(C)(Oc1ccc(C(=O)c2ccc(Cl)cc2)cc1)C(=O)NCCCCNC(=O)/C=C\C(=O)O] | C₂₅H₂₇ClN₂O₆ | 486.94 |
| PP17 | 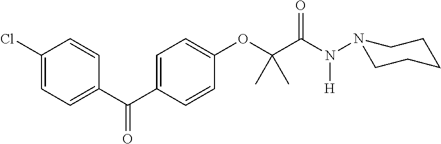 [SMILES: CC(C)(Oc1ccc(C(=O)c2ccc(Cl)cc2)cc1)C(=O)NN1CCCCC1] | C₂₂H₂₅ClN₂O₃ | 400.90 |
| PP18 | 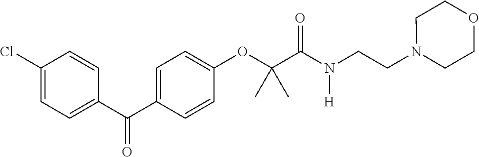 [SMILES: CC(C)(Oc1ccc(C(=O)c2ccc(Cl)cc2)cc1)C(=O)NCCN1CCOCC1] | C₂₃H₂₇ClN₂O₄ | 430.92 |
| PP19 | 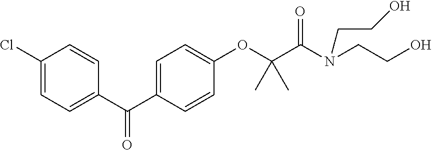 [SMILES: CC(C)(Oc1ccc(C(=O)c2ccc(Cl)cc2)cc1)C(=O)N(CCO)CCO] | C₂₁H₂₄ClNO₅ | 405.87 |
| PP20 | 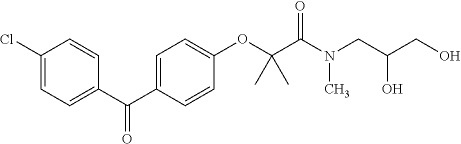 [SMILES: CN(CC(O)CO)C(=O)C(C)(C)Oc1ccc(C(=O)c2ccc(Cl)cc2)cc1] | C₂₁H₂₄ClNO₅ | 405.87 |
| PP21 | 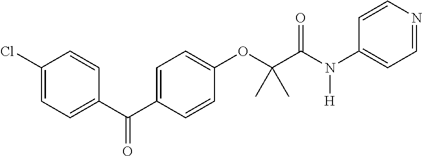 [SMILES: CC(C)(Oc1ccc(C(=O)c2ccc(Cl)cc2)cc1)C(=O)Nc1ccncc1] | C₂₂H₁₉ClN₂O₃ | 394.85 |
| PP22 | 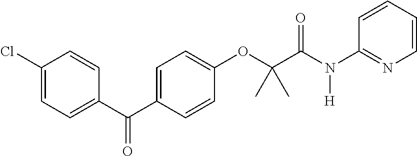 [SMILES: CC(C)(Oc1ccc(C(=O)c2ccc(Cl)cc2)cc1)C(=O)Nc1ccccn1] | C₂₂H₁₉ClN₂O₃ | 394.85 |
| PP23 | 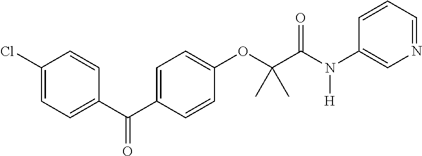 [SMILES: CC(C)(Oc1ccc(C(=O)c2ccc(Cl)cc2)cc1)C(=O)Nc1cccnc1] | C₂₂H₁₉ClN₂O₃ | 394.85 |
| PP24 | 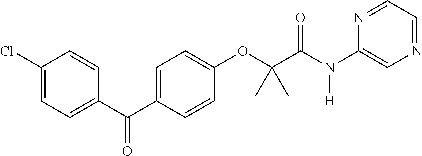 [SMILES: CC(C)(Oc1ccc(C(=O)c2ccc(Cl)cc2)cc1)C(=O)Nc1cnccn1] | C₂₁H₁₈ClN₃O₃ | 395.84 |
| PP25 | 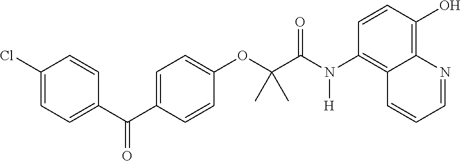 [SMILES: CC(C)(Oc1ccc(C(=O)c2ccc(Cl)cc2)cc1)C(=O)Nc1ccc(O)c2ncccc12] | C₂₆H₂₁ClN₂O₄ | 460.91 |
| PP26 | 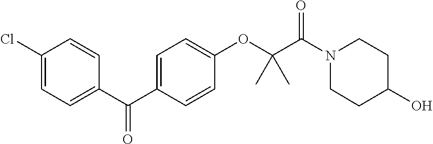 [SMILES: CC(C)(Oc1ccc(C(=O)c2ccc(Cl)cc2)cc1)C(=O)N1CCC(O)CC1] | C₂₂H₂₄ClNO₄ | 401.88 |
| PP27 | 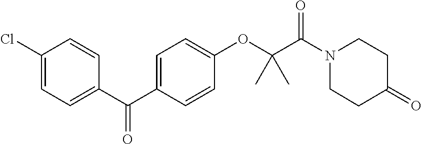 [SMILES: CC(C)(Oc1ccc(C(=O)c2ccc(Cl)cc2)cc1)C(=O)N1CCC(=O)CC1] | C₂₂H₂₂ClNO₄ | 399.87 |
| PP28 | 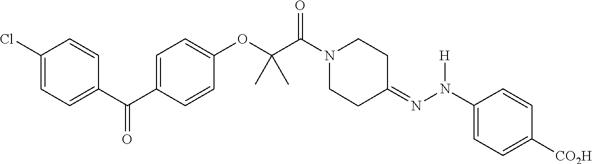 [SMILES: CC(C)(Oc1ccc(C(=O)c2ccc(Cl)cc2)cc1)C(=O)N1CCC(=NNc2ccc(C(=O)O)cc2)CC1] | C₂₉H₂₈ClN₃O₅ | 534.00 |
| PP29 | 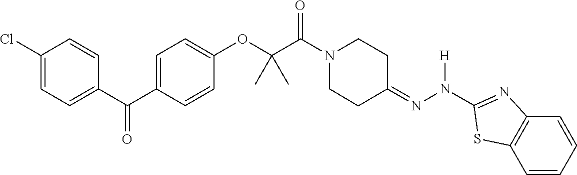 [SMILES: CC(C)(Oc1ccc(C(=O)c2ccc(Cl)cc2)cc1)C(=O)N1CCC(=NNc2nc3ccccc3s2)CC1] | C₂₉H₂₇ClN₄O₃S | 547.07 |
| PP30 | 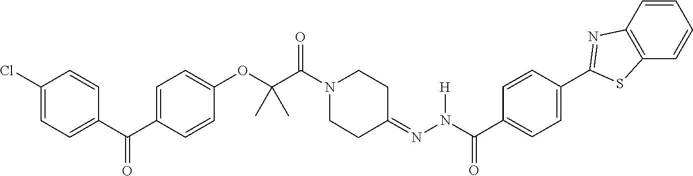 [SMILES: CC(C)(Oc1ccc(C(=O)c2ccc(Cl)cc2)cc1)C(=O)N1CCC(=NNC(=O)c2ccc(-c3nc4ccccc4s3)cc2)CC1] | C₃₆H₃₁ClN₄O₄S | 651.17 |
| PP31 | 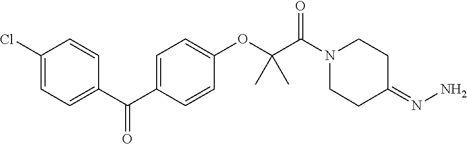 [SMILES: CC(C)(Oc1ccc(C(=O)c2ccc(Cl)cc2)cc1)C(=O)N1CCC(=NN)CC1] | C₂₂H₂₄ClN₃O₃ | 413.90 |
| PP32 | 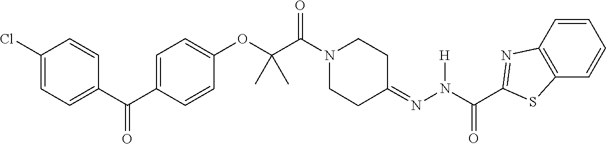 [SMILES: CC(C)(Oc1ccc(C(=O)c2ccc(Cl)cc2)cc1)C(=O)N1CCC(=NNC(=O)c2nc3ccccc3s2)CC1] | C₃₀H₂₇ClN₄O₄S | 575.08 |
| PP33 | 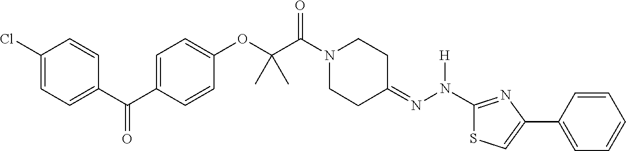 [SMILES: CC(C)(Oc1ccc(C(=O)c2ccc(Cl)cc2)cc1)C(=O)N1CCC(=NNc2nc(-c3ccccc3)cs2)CC1] | C₃₁H₂₉ClN₄O₃S | 573.11 |
| PP34 | 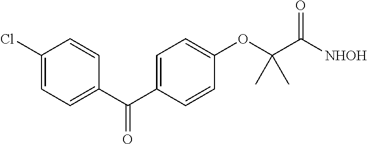 | C₁₇H₁₆ClNO₄ | 333.77 |
| PP35 | 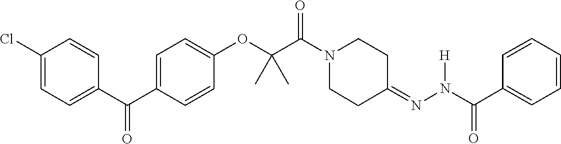 [SMILES: CC(C)(Oc1ccc(C(=O)c2ccc(Cl)cc2)cc1)C(=O)N1CCC(=NNC(=O)c2ccccc2)CC1] | C₂₉H₂₈ClN₃O₄ | 518.00 |
| PP36 | 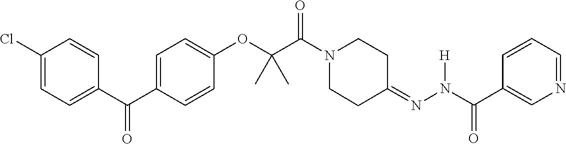 [SMILES: CC(C)(Oc1ccc(C(=O)c2ccc(Cl)cc2)cc1)C(=O)N1CCC(=NNC(=O)c2cccnc2)CC1] | C₂₈H₂₇ClN₄O₄ | 518.99 |
| PP37 | 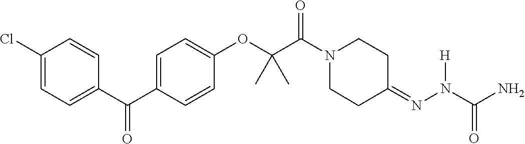 [SMILES: CC(C)(Oc1ccc(C(=O)c2ccc(Cl)cc2)cc1)C(=O)N1CCC(=NNC(N)=O)CC1] | C₂₃H₂₅ClN₄O₄ | 456.92 |
| PP38 | 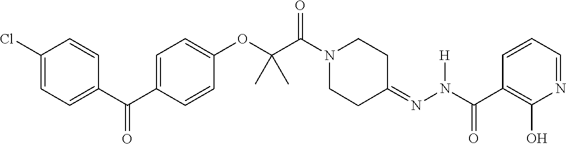 [SMILES: CC(C)(Oc1ccc(C(=O)c2ccc(Cl)cc2)cc1)C(=O)N1CCC(=NNC(=O)c2cccnc2O)CC1] | C₂₈H₂₇ClN₄O₅ | 534.99 |
| PP39 | 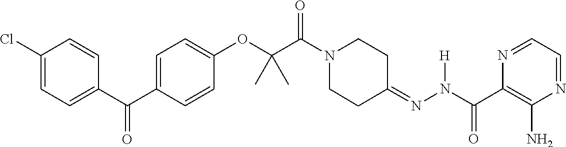 [SMILES: CC(C)(Oc1ccc(C(=O)c2ccc(Cl)cc2)cc1)C(=O)N1CCC(=NNC(=O)c2nccnc2N)CC1] | C₂₇H₂₇ClN₆O₄ | 534.99 |
| PP40 | 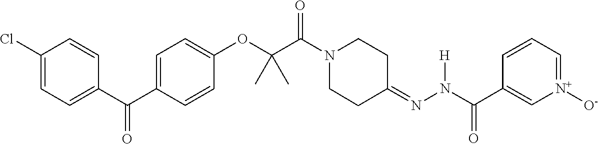 [SMILES: CC(C)(Oc1ccc(C(=O)c2ccc(Cl)cc2)cc1)C(=O)N1CCC(=NNC(=O)c2ccc[n+]([O-])c2)CC1] | C₂₈H₂₇ClN₄O₅ | 534.99 |
| PP41 | 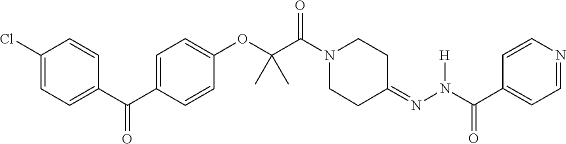 [SMILES: CC(C)(Oc1ccc(C(=O)c2ccc(Cl)cc2)cc1)C(=O)N1CCC(=NNC(=O)c2ccncc2)CC1] | C₂₈H₂₇ClN₄O₄ | 518.99 |
| PP42 | 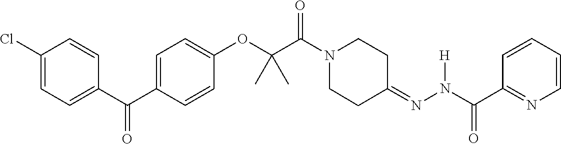 [SMILES: CC(C)(Oc1ccc(C(=O)c2ccc(Cl)cc2)cc1)C(=O)N1CCC(=NNC(=O)c2ccccn2)CC1] | C₂₈H₂₇ClN₄O₄ | 518.99 |
| PP43 | 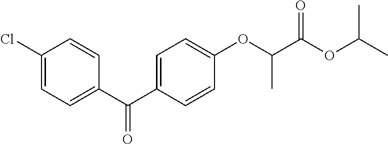 [SMILES: CC(C)OC(=O)C(C)Oc1ccc(C(=O)c2ccc(Cl)cc2)cc1] | C₁₉H₁₉ClO₄ | 346.80 |
| PP44 | 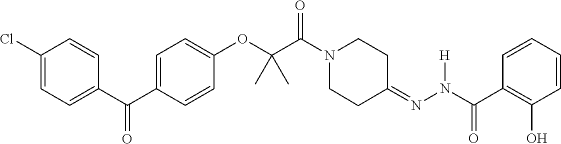 [SMILES: CC(C)(Oc1ccc(C(=O)c2ccc(Cl)cc2)cc1)C(=O)N1CCC(=NNC(=O)c2ccccc2O)CC1] | C₂₉H₂₈ClN₃O₅ | 534.00 |
| PP45 | 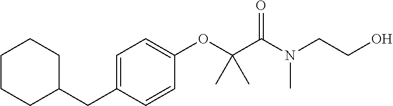 [SMILES: CN(CCO)C(=O)C(C)(C)Oc1ccc(CC2CCCCC2)cc1] | C₂₀H₃₁NO₃ | 333.47 |
| PP46 | 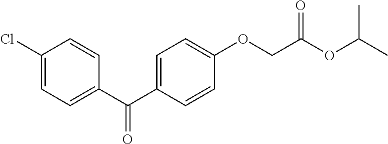 [SMILES: CC(C)OC(=O)COc1ccc(C(=O)c2ccc(Cl)cc2)cc1] | C₁₈H₁₇ClO₄ | 332.78 |
| PP47 | 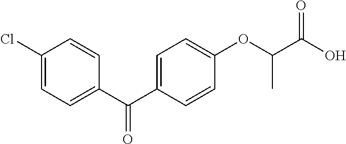 [SMILES: CC(Oc1ccc(C(=O)c2ccc(Cl)cc2)cc1)C(=O)O] | C₁₆H₁₃ClO₄ | 304.72 |
| PP48 | 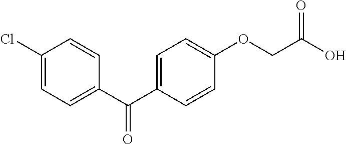 [SMILES: O=C(O)COc1ccc(C(=O)c2ccc(Cl)cc2)cc1] | C₁₅H₁₁ClO₄ | 290.70 |
| PP49 | 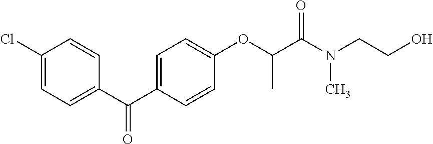 [SMILES: CC(Oc1ccc(C(=O)c2ccc(Cl)cc2)cc1)C(=O)N(C)CCO] | C₁₉H₂₀ClNO₄ | 361.82 |
| PP50 | 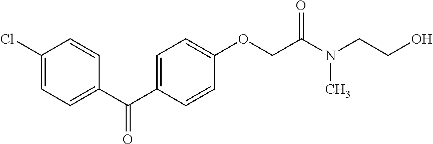 [SMILES: CN(CCO)C(=O)COc1ccc(C(=O)c2ccc(Cl)cc2)cc1] | C₁₈H₁₈ClNO₄ | 347.79 |
| PP51 | 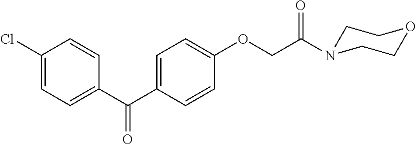 [SMILES: O=C(c1ccc(Cl)cc1)c1ccc(OCC(=O)N2CCOCC2)cc1] | C₁₉H₁₈ClNO₄ | 359.80 |
| PP52 | 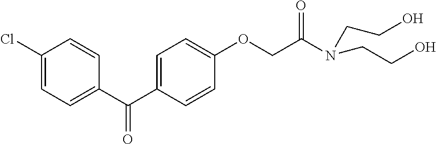 [SMILES: O=C(c1ccc(Cl)cc1)c1ccc(OCC(=O)N(CCO)CCO)cc1] | C₁₉H₂₀ClNO₅ | 377.82 |
| PP53 | 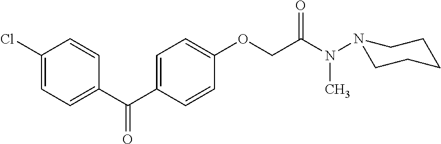 [SMILES: CN(C(=O)COc1ccc(C(=O)c2ccc(Cl)cc2)cc1)N1CCCCC1] | C₂₁H₂₃ClN₂O | 386.87 |
| PP54 | 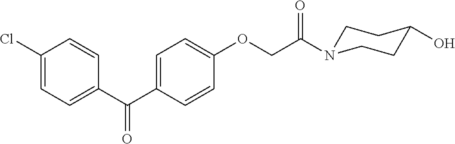 [SMILES: O=C(c1ccc(Cl)cc1)c1ccc(OCC(=O)N2CCC(O)CC2)cc1] | C₂₀H₂₀ClNO₄ | 373.83 |
| PP55 | 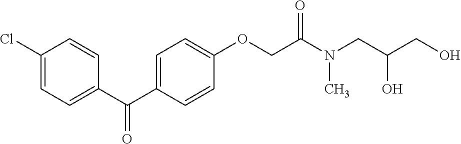 [SMILES: CN(CC(O)CO)C(=O)COc1ccc(C(=O)c2ccc(Cl)cc2)cc1] | C₁₉H₂₀ClNO₅ | 377.82 |
| PP56 | 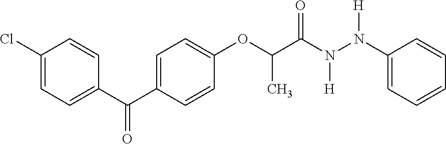 [SMILES: CC(Oc1ccc(C(=O)c2ccc(Cl)cc2)cc1)C(=O)NNc1ccccc1] | C₂₂H₁₉ClN₂O₃ | 394.85 |
| PP57 | 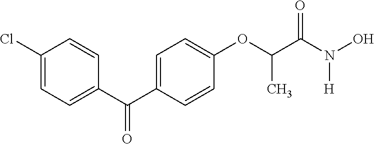 [SMILES: CC(Oc1ccc(C(=O)c2ccc(Cl)cc2)cc1)C(=O)NO] | C₁₆H₁₄ClNO₄ | 319.74 |
| PP58 | 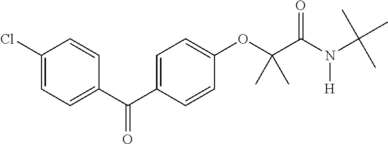 [SMILES: CC(C)(C)NC(=O)C(C)(C)Oc1ccc(C(=O)c2ccc(Cl)cc2)cc1] | C₂₁H₂₄ClNO₃ | 373.87 |
| PP59 | 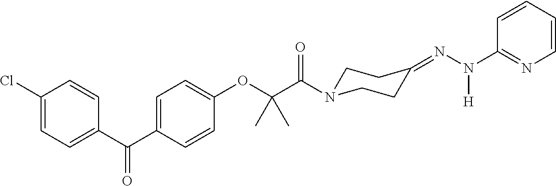 [SMILES: CC(C)(Oc1ccc(C(=O)c2ccc(Cl)cc2)cc1)C(=O)N1CCC(=NNc2ccccn2)CC1] | C₂₇H₂₇ClN₄O₃ | 490.98 |
| PP60 | 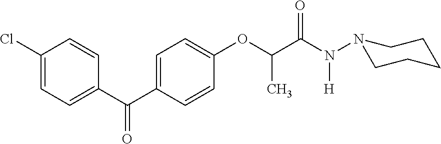 [SMILES: CC(Oc1ccc(C(=O)c2ccc(Cl)cc2)cc1)C(=O)NN1CCCCC1] | C₂₁H₂₃ClN₂O₃ | 386.87 |
| PP61 | 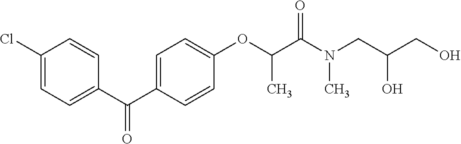 [SMILES: CC(Oc1ccc(C(=O)c2ccc(Cl)cc2)cc1)C(=O)N(C)CC(O)CO] | C₂₀H₂₂ClNO₅ | 391.85 |
| PP62 | 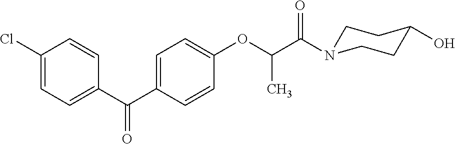 [SMILES: CC(Oc1ccc(C(=O)c2ccc(Cl)cc2)cc1)C(=O)N1CCC(O)CC1] | C₂₁H₂₂ClNO₄ | 387.86 |
| PP63 | 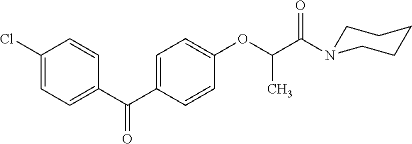 [SMILES: CC(Oc1ccc(C(=O)c2ccc(Cl)cc2)cc1)C(=O)N1CCCCC1] | C₂₁H₂₂ClNO₃ | 371.86 |
| PP64 | 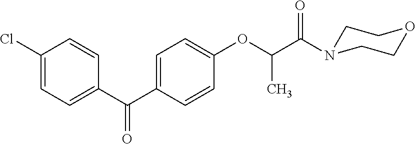 [SMILES: CC(Oc1ccc(C(=O)c2ccc(Cl)cc2)cc1)C(=O)N1CCOCC1] | C₂₀H₂₀ClNO₄ | 373.83 |
| PP65 | 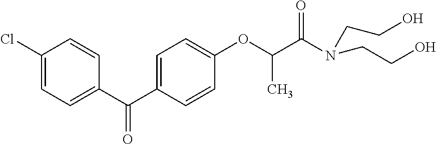 [SMILES: CC(Oc1ccc(C(=O)c2ccc(Cl)cc2)cc1)C(=O)N(CCO)CCO] | C₂₀H₂₂ClNO₅ | 391.85 |
| PP66 | 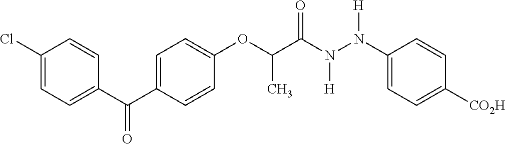 [SMILES: CC(Oc1ccc(C(=O)c2ccc(Cl)cc2)cc1)C(=O)NNc1ccc(C(=O)O)cc1] | C₂₃H₁₉ClN₂O₅ | 438.86 |
| PP67 | 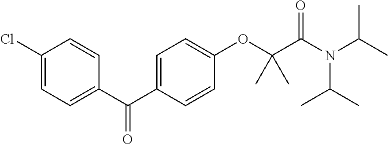 [SMILES: CC(C)N(C(=O)C(C)(C)Oc1ccc(C(=O)c2ccc(Cl)cc2)cc1)C(C)C] | C₂₃H₂₈ClNO₃ | 401.93 |
| PP68 | 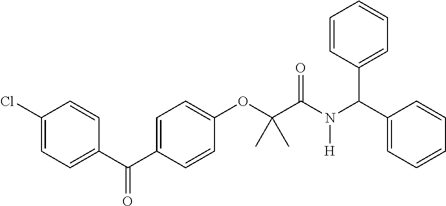 [SMILES: CC(C)(Oc1ccc(C(=O)c2ccc(Cl)cc2)cc1)C(=O)NC(c1ccccc1)c1ccccc1] | C₃₀H₂₆ClNO₃ | 483.99 |
| PP69 | 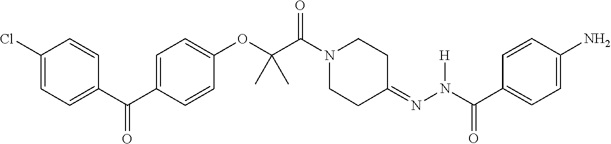 [SMILES: CC(C)(Oc1ccc(C(=O)c2ccc(Cl)cc2)cc1)C(=O)N1CCC(=NNC(=O)c2ccc(N)cc2)CC1] | C₂₉H₂₉CIN₄O₄ | 533.02 |
| PP70 | 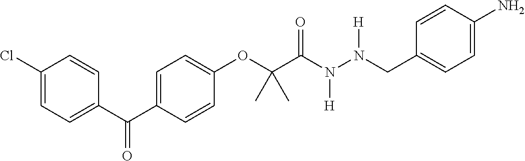 [SMILES: CC(C)(Oc1ccc(C(=O)c2ccc(Cl)cc2)cc1)C(=O)NNCc1ccc(N)cc1] | C₂₄H₂₂ClN₃O₄ | 451.90 |
| PP71 | 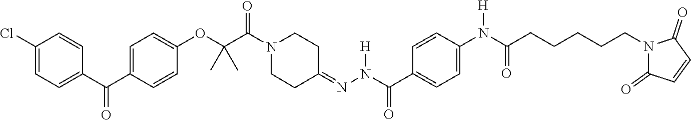 [SMILES: CC(C)(Oc1ccc(C(=O)c2ccc(Cl)cc2)cc1)C(=O)N1CCC(=NNC(=O)c2ccc(NC(=O)CCCCCN3C(=O)C=CC3=O)cc2)CC1] | C₃₉H₄₀ClN₅O₇ | 726.22 |
| PP72 | 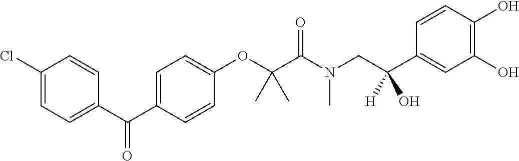 [SMILES: CN(C[C@H](O)c1ccc(O)c(O)c1)C(=O)C(C)(C)Oc1ccc(C(=O)c2ccc(Cl)cc2)cc1] | C₂₆H₂₆ClNO₆ | 483.94 |
| PP73 | 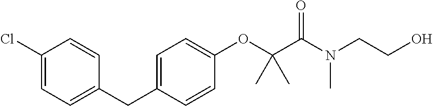 [SMILES: CN(CCO)C(=O)C(C)(C)Oc1ccc(Cc2ccc(Cl)cc2)cc1] | C₂₀H₂₂ClNO₄ | 375.85 |

Aspects of the invention are also directed towards an anticancer compound comprising the following formula:

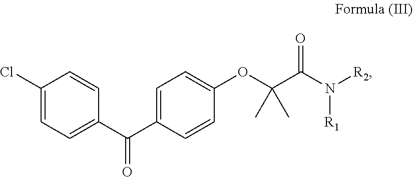

For example, R₁ can be H, alkyl, hydroxyalkyl. In embodiments, R₂ can be H, alkyl, hydroxyalkyl, polyhydroxyalkyl, aminoalkyl, dialkylaminoalkyl. In embodiments, R₁-R₂ can be (CH₂)ₙ, (CH₂)ₙO(CH₂)ₘ, (CH₂)ₙCHOH(CH₂)ₘ, (CH₂)ₙNCH₃(CH₂)ₘ, where n and m are 1, 2, 3, 4, or 5.

Non-limiting examples of the anticancer compound of Formula (III) comprise the compounds in Table 3.

For example, the anticancer compound of Formula (III) can be:

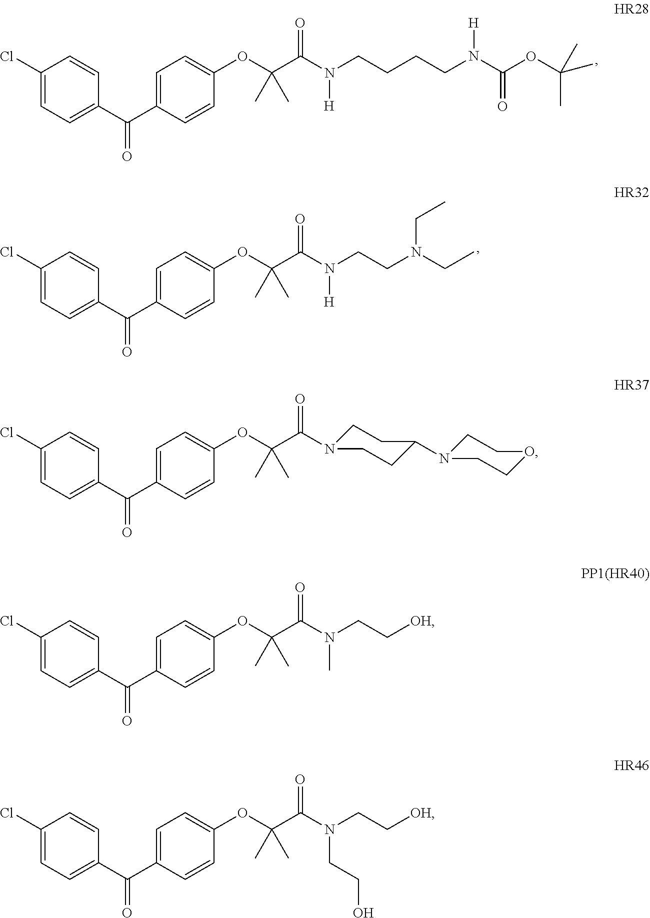 [SMILES: CC(C)(C)OC(=O)NCCCCNC(=O)C(C)(C)Oc1ccc(C(=O)c2ccc(Cl)cc2)cc1 ; CCN(CC)CCNC(=O)C(C)(C)Oc1ccc(C(=O)c2ccc(Cl)cc2)cc1 ; CC(C)(Oc1ccc(C(=O)c2ccc(Cl)cc2)cc1)C(=O)N1CCC(N2CCOCC2)CC1 ; CN(CCO)C(=O)C(C)(C)Oc1ccc(C(=O)c2ccc(Cl)cc2)cc1 ; CC(C)(Oc1ccc(C(=O)c2ccc(Cl)cc2)cc1)C(=O)N(CCO)CCO]

a pharmaceutically acceptable salt thereof, or a derivative thereof.

Non-limiting examples of compounds of the invention comprise those compounds of Table 3.

Table 3 refers to compounds of the invention:

| Com- | | | | | |
|---|---|---|---|---|---|
| pound | Structure | Formula | MW | CAS | Reference |
| AA | 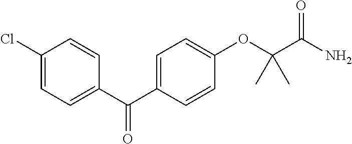 [SMILES: CC(C)(Oc1ccc(C(=O)c2ccc(Cl)cc2)cc1)C(N)=O] | C₁₇H₁₆ClNO₃ | 317.77 | NONE | NONE |
| MA | 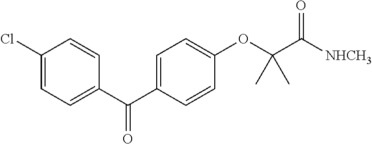 [SMILES: CNC(=O)C(C)(C)Oc1ccc(C(=O)c2ccc(Cl)cc2)cc1] | C₁₈H₁₈ClNO₃ | 331.79 | NONE | NONE |
| DMA | 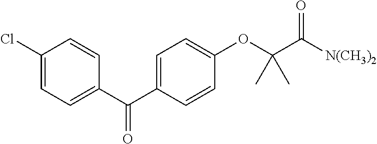 [SMILES: CN(C)C(=O)C(C)(C)Oc1ccc(C(=O)c2ccc(Cl)cc2)cc1] | C₁₉H₂₀ClNO₃ | 345.82 | NONE | NONE |
| HR1 (PP45) | 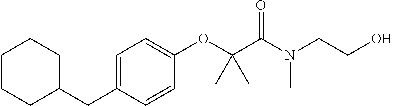 [SMILES: CN(CCO)C(=O)C(C)(C)Oc1ccc(CC2CCCCC2)cc1] | C₂₀H₃₁NO₃ | 333.47 | NONE | NONE |
| HR2 (PP73) | 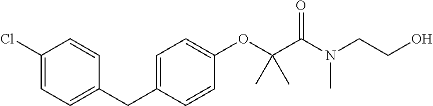 [SMILES: CN(CCO)C(=O)C(C)(C)Oc1ccc(Cc2ccc(Cl)cc2)cc1] | C₂₀H₂₂ClNO₄ | 375.85 | NONE | NONE |
| HR3 (PP76) | 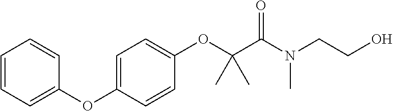 [SMILES: CN(CCO)C(=O)C(C)(C)Oc1ccc(Oc2ccccc2)cc1] | C₁₉H₂₃NO₄ | 329.39 | NONE | NONE |
| HR4 (PP89) | 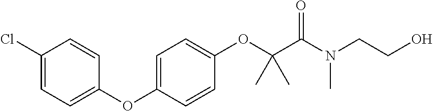 [SMILES: CN(CCO)C(=O)C(C)(C)Oc1ccc(Oc2ccc(Cl)cc2)cc1] | C₁₉H₂₂ClNO₄ | 363.84 | NONE | NONE |
| HR5 (PP90) | 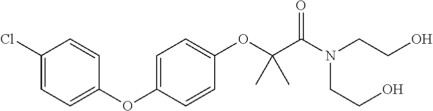 [SMILES: CC(C)(Oc1ccc(Oc2ccc(Cl)cc2)cc1)C(=O)N(CCO)CCO] | C₂₀H₂₄ClNO₅ | 393.86 | NONE | NONE |
| HR6 (PP78) | 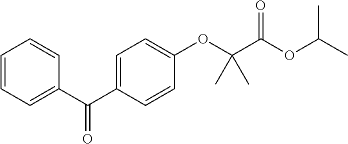 [SMILES: CC(C)OC(=O)C(C)(C)Oc1ccc(C(=O)c2ccccc2)cc1] | C₂₀H₂₂O₄ | 326.39 | 42019-13-6 | Mieville, A. U.S. Pat. No. 4,235,896 (1980). |
| HR7 (PP80) | 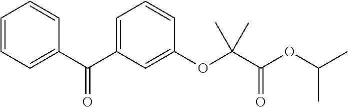 [SMILES: CC(C)OC(=O)C(C)(C)Oc1cccc(C(=O)c2ccccc2)c1] | C₂₀H₂₂O₄ | 326.39 | 62809-73 | Majoic, B. U.S. Pat. No. 4,146,385 (1979). |
| HR8 (PP77) | 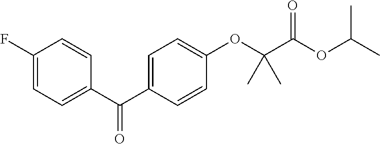 [SMILES: CC(C)OC(=O)C(C)(C)Oc1ccc(C(=O)c2ccc(F)cc2)cc1] | C₂₀H₂₁FO₄ | 344.38 | 61002-28-6 | Schimler, et al.; Journal of the American Chemical Society, 2017, 137, 1452-1455. |
| HR9 (PP92) | 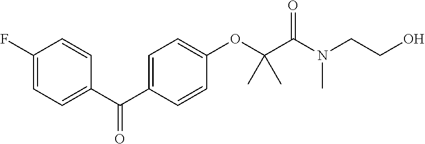 [SMILES: CN(CCO)C(=O)C(C)(C)Oc1ccc(C(=O)c2ccc(F)cc2)cc1] | C₂₀H₂₂FNO₄ | 359.39 | NONE | NONE |
| HR10 (PP93) | 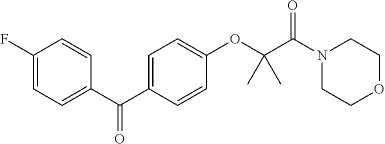 [SMILES: CC(C)(Oc1ccc(C(=O)c2ccc(F)cc2)cc1)C(=O)N1CCOCC1] | C₂₁H₂₂FNO₄ | 371.40 | NONE | NONE |
| HR11 (PP91) | 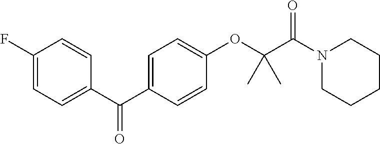 [SMILES: CC(C)(Oc1ccc(C(=O)c2ccc(F)cc2)cc1)C(=O)N1CCCCC1] | C₂₂H₂₄FNO₃ | 369.43 | NONE | NONE |
| HR12 (PP46) | 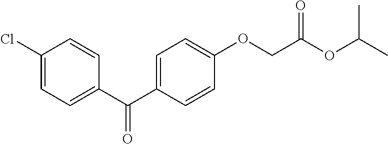 [SMILES: CC(C)OC(=O)COc1ccc(C(=O)c2ccc(Cl)cc2)cc1] | C₁₈H₁₇ClO₄ | 332.78 | NONE | NONE |
| HR13 (PP50) | 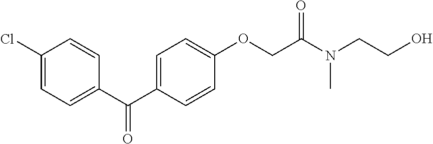 [SMILES: CN(CCO)C(=O)COc1ccc(C(=O)c2ccc(Cl)cc2)cc1] | C₁₈H₁₈ClNO₄ | 347.79 | NONE | NONE |
| HR14 (PP52) | 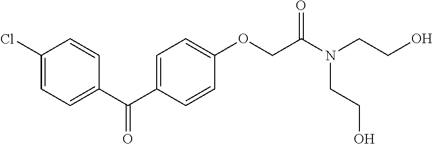 [SMILES: O=C(c1ccc(Cl)cc1)c1ccc(OCC(=O)N(CCO)CCO)cc1] | C₁₉H₂₀ClNO₅ | 377.82 | NONE | NONE |
| HR15 (PP55) | 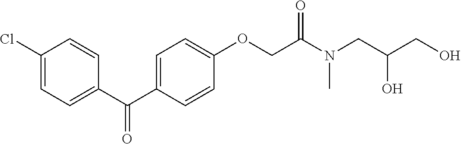 [SMILES: CN(CC(O)CO)C(=O)COc1ccc(C(=O)c2ccc(Cl)cc2)cc1] | C₁₉H₂₀ClNO₅ | 377.82 | NONE | NONE |
| HR16 (PP54) | 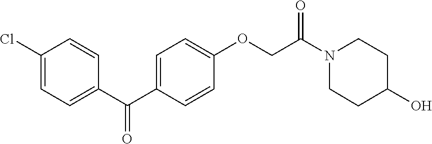 [SMILES: O=C(c1ccc(Cl)cc1)c1ccc(OCC(=O)N2CCC(O)CC2)cc1] | C₂₀H₂₀ClNO₄ | 373.83 | NONE | NONE |
| HR17 (PP51) | 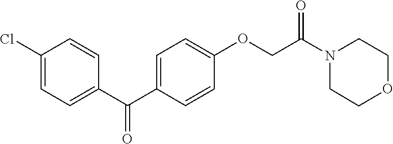 [SMILES: O=C(c1ccc(Cl)cc1)c1ccc(OCC(=O)N2CCOCC2)cc1] | C₁₉H₁₈ClNO₄ | 359.80 | NONE | NONE |
| HR18 (PP49) | 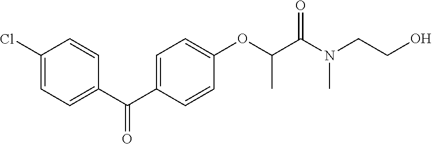 [SMILES: CC(Oc1ccc(C(=O)c2ccc(Cl)cc2)cc1)C(=O)N(C)CCO] | C₁₉H₂₀ClNO₄ | 361.82 | NONE | NONE |
| HR19 (PP65) | 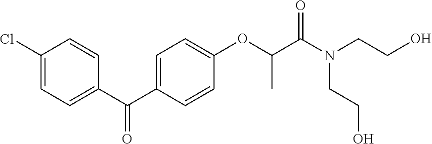 [SMILES: CC(Oc1ccc(C(=O)c2ccc(Cl)cc2)cc1)C(=O)N(CCO)CCO] | C₂₀H₂₂ClNO₅ | 391.85 | NONE | NONE |
| HR20 (PP61) | 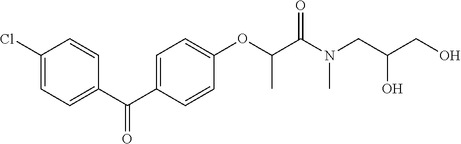 [SMILES: CC(Oc1ccc(C(=O)c2ccc(Cl)cc2)cc1)C(=O)N(C)CC(O)CO] | C₂₀H₂₂ClNO₅ | 391.85 | NONE | NONE |
| HR21 (PP62) | 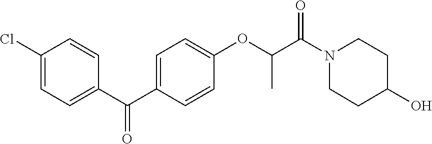 [SMILES: CC(Oc1ccc(C(=O)c2ccc(Cl)cc2)cc1)C(=O)N1CCC(O)CC1] | C₂₁H₂₂ClNO₄ | 387.86 | NONE | NONE |
| HR22 (PP63) | 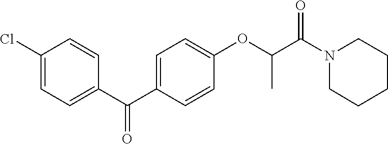 [SMILES: CC(Oc1ccc(C(=O)c2ccc(Cl)cc2)cc1)C(=O)N1CCCCC1] | C₂₁H₂₂ClNO₃ | 371.86 | NONE | NONE |
| HR23 (PP64) | 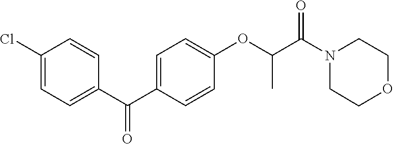 [SMILES: CC(Oc1ccc(C(=O)c2ccc(Cl)cc2)cc1)C(=O)N1CCOCC1] | C₂₀H₂₀ClNO₄ | 373.83 | NONE | NONE |
| HR24 (PP58) | 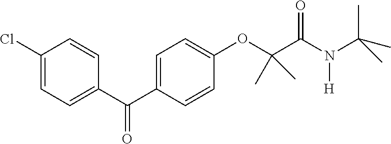 [SMILES: CC(C)(C)NC(=O)C(C)(C)Oc1ccc(C(=O)c2ccc(Cl)cc2)cc1] | C₂₁H₂₄ClNO₃ | 373.87 | NONE | NONE |
| HR25 (PP67) | 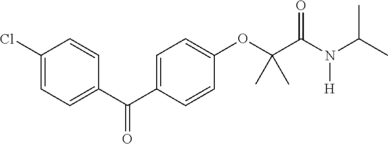 [SMILES: CC(C)NC(=O)C(C)(C)Oc1ccc(C(=O)c2ccc(Cl)cc2)cc1] | C₂₃H₂₈ClNO₃ | 359.85 | NONE | NONE |
| HR26 (PP5) | 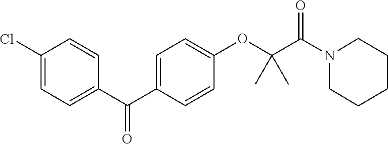 [SMILES: CC(C)(Oc1ccc(C(=O)c2ccc(Cl)cc2)cc1)C(=O)N1CCCCC1] | C₂₂H₂₄ClNO₃ | 385.88 | 42019-09-9 | Mieville, A. U.S. Pat. No. 4,235,896 (1980). |
| HR27 (PP68) | 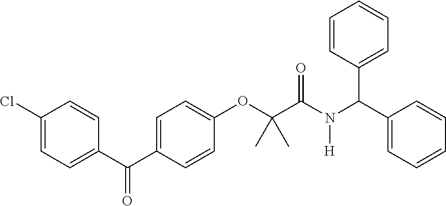 [SMILES: CC(C)(Oc1ccc(C(=O)c2ccc(Cl)cc2)cc1)C(=O)NC(c1ccccc1)c1ccccc1] | C₃₀H₂₆ClNO₃ | 483.99 | NONE | NONE |
| HR28 (PP14) | 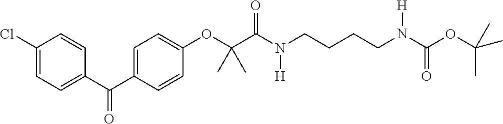 [SMILES: CC(C)(C)OC(=O)NCCCCNC(=O)C(C)(C)Oc1ccc(C(=O)c2ccc(Cl)cc2)cc1] | C₂₆H₃₃ClN₂O₅ | 489.00 | NONE | NONE |
| HR29 (PP8) | 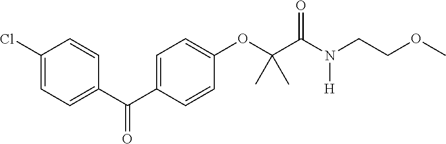 [SMILES: COCCNC(=O)C(C)(C)Oc1ccc(C(=O)c2ccc(Cl)cc2)cc1] | C₂₀H₂₂ClNO₄ | 375.85 | NONE | NONE |
| HR30 (PP9) | 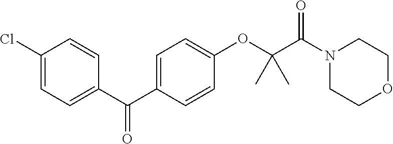 [SMILES: CC(C)(Oc1ccc(C(=O)c2ccc(Cl)cc2)cc1)C(=O)N1CCOCC1] | C₂₁H₂₂ClNO₄ | 387.86 | 42019-10-3 | Mieville, A. U.S. Pat. No. 4,235,896 (1980). |
| HR31 (PP15) | 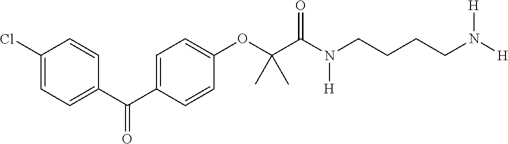 [SMILES: CC(C)(Oc1ccc(C(=O)c2ccc(Cl)cc2)cc1)C(=O)NCCCCN] | C₂₁H₂₅ClN₂O₃ | 388.89 | NONE | NONE |
| HR32 (PP10) | 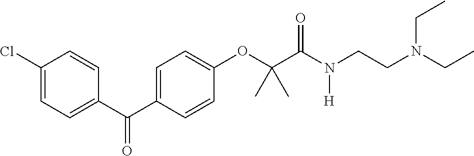 [SMILES: CCN(CC)CCNC(=O)C(C)(C)Oc1ccc(C(=O)c2ccc(Cl)cc2)cc1] | C₂₅H₃₃ClN₂O₃ | 444.99 | 61002-39-9 | Mieville, A. U.S. Pat. No. 4,235,896 (1980). |
| HR33 (PP18) | 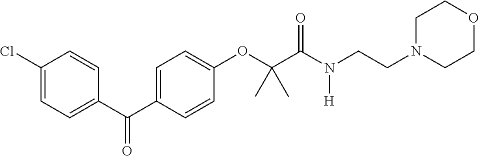 [SMILES: CC(C)(Oc1ccc(C(=O)c2ccc(Cl)cc2)cc1)C(=O)NCCN1CCOCC1] | C₂₃H₂₇ClN₂O₄ | 430.92 | NONE | NONE |
| HR34 (PP2) | 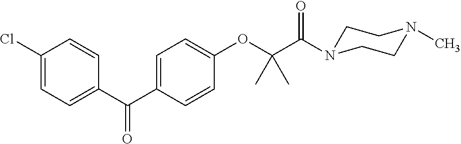 [SMILES: CN1CCN(C(=O)C(C)(C)Oc2ccc(C(=O)c3ccc(Cl)cc3)cc2)CC1] | C₂₂H₂₅ClN₂O₃ | 400.90 | NONE | NONE |
| HR35 (PP2HCl) | 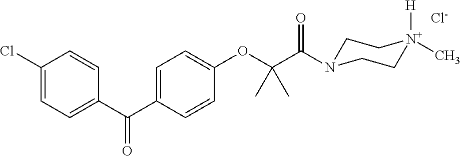 [SMILES: C[NH+]1CCN(C(=O)C(C)(C)Oc2ccc(C(=O)c3ccc(Cl)cc3)cc2)CC1 ; [Cl-]] | C₂₂H₂₆Cl₂N₂O₃ | 437.36 | NONE | NONE |
| HR36 (PP2Mel) | 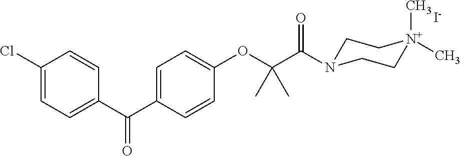 [SMILES: CC(C)(Oc1ccc(C(=O)c2ccc(Cl)cc2)cc1)C(=O)N1CC[N+](C)(C)CC1 ; [I-]] | C₂₃H₂₈ClIN₂O₃ | 542.84 | NONE | NONE |
| HR37 (PP4) | 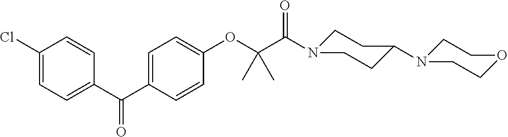 [SMILES: CC(C)(Oc1ccc(C(=O)c2ccc(Cl)cc2)cc1)C(=O)N1CCC(N2CCOCC2)CC1] | C₂₆H₃₁ClN₂O₄ | 470.99 | NONE | NONE |
| HR38 (PP4HCl) | 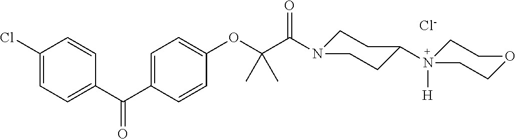 | C₂₆H₃₂Cl₂N₂O₄ | 507.45 | NONE | NONE |
| HR39 (PP7) | 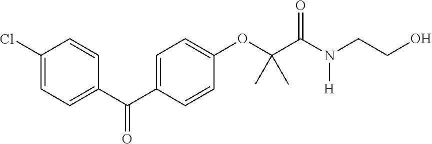 [SMILES: CC(C)(Oc1ccc(C(=O)c2ccc(Cl)cc2)cc1)C(=O)NCCO] | C₁₉H₂₀ClNO₄ | 361.82 | NONE | NONE |
| HR40 (PP1) | 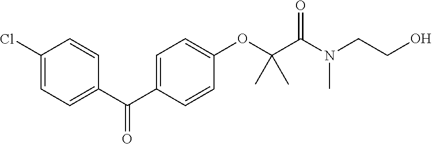 [SMILES: CN(CCO)C(=O)C(C)(C)Oc1ccc(C(=O)c2ccc(Cl)cc2)cc1] | C₂₀H₂₂ClNO₄ | 375.85 | NONE | NONE |
| HR41 (PP26) | 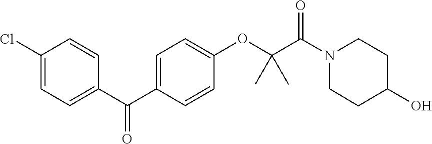 [SMILES: CC(C)(Oc1ccc(C(=O)c2ccc(Cl)cc2)cc1)C(=O)N1CCC(O)CC1] | C₂₂H₂₄ClNO₄ | 401.88 | NONE | NONE |
| HR42 (PP13) | 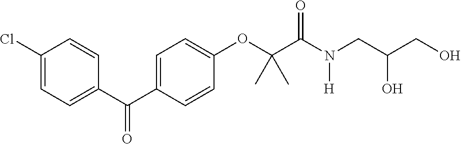 [SMILES: CC(C)(Oc1ccc(C(=O)c2ccc(Cl)cc2)cc1)C(=O)NCC(O)CO] | C₂₀H₂₂ClNO₅ | 391.85 | NONE | NONE |
| HR43 (PP11) | 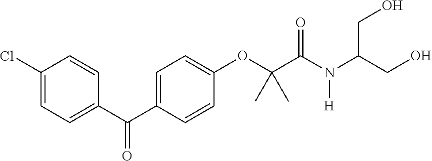 [SMILES: CC(C)(Oc1ccc(C(=O)c2ccc(Cl)cc2)cc1)C(=O)NC(CO)CO] | C₂₀H₂₂ClNO₅ | 391.85 | NONE | NONE |
| HR44 (PP20) | 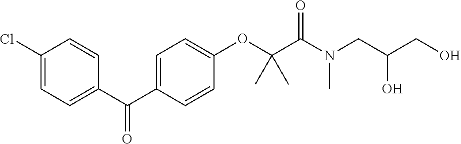 [SMILES: CN(CC(O)CO)C(=O)C(C)(C)Oc1ccc(C(=O)c2ccc(Cl)cc2)cc1] | C₂₁H₂₄ClNO₅ | 405.87 | NONE | NONE |
| HR45 (PP6) | 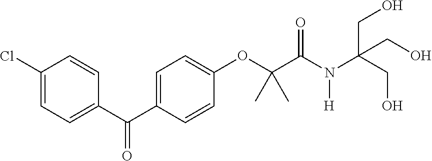 [SMILES: CC(C)(Oc1ccc(C(=O)c2ccc(Cl)cc2)cc1)C(=O)NC(CO)(CO)CO] | C₂₁H₂₄ClNO₆ | 421.87 | NONE | NONE |
| HR46 (PP19) | 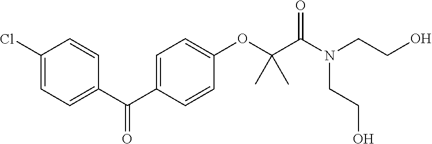 [SMILES: CC(C)(Oc1ccc(C(=O)c2ccc(Cl)cc2)cc1)C(=O)N(CCO)CCO] | C₂₁H₂₄ClNO₅ | 405.87 | 72678-26-3 | Borzatta, et al. U.S. Pat. No. 4,378,373 (1983). |
| HR47 (PP3) | 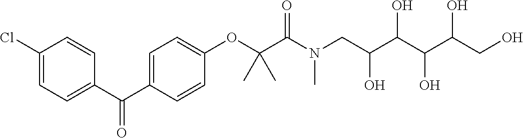 [SMILES: CN(CC(O)C(O)C(O)C(O)CO)C(=O)C(C)(C)Oc1ccc(C(=O)c2ccc(Cl)cc2)cc1] | C₂₄H₃₀ClNO₈ | 495.95 | NONE | NONE |

See also, for example, U.S. Pat. No. 4,235,896 (1980); U.S. Pat. No. 4,378,373 (1983); United State Patent Application US 20090054450A1; Yu, et al. European Journal of Medicinal Chemistry 2015, 106, 50-59; U.S. Pat. No. 4,146,385 (1979); Hogberg, T., et al. Acta Pharmaceutica Suecica 1976, 13, 427-438; Schimler, S. D., et al. Journal of the American Chemical Society, 2017, 137, 1452-1455; Giampietro, Letizia, et al. Medicinal Chemistry, 2014, 10, 59-65, each of which are incorporated by reference herein in their entireties.

Aspects of the invention are also directed towards an anti cancer compound comprising the following formula:

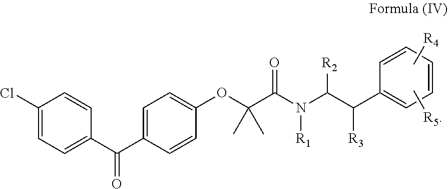

For example, R₁ can be H or CH₃; R₂ can be OH, OCH₃, or any combination thereof; R₃ can be OH, OCH₃, or any combination thereof; R₄ can be OH, OCH₃, or any combination thereof; R₅ can be OH, OCH₃, or any combination thereof.

Non-limiting examples of anti cancer compounds of Formula (IV) can be found in Example 4. For example, the anti cancer compound can be any of the following structures:

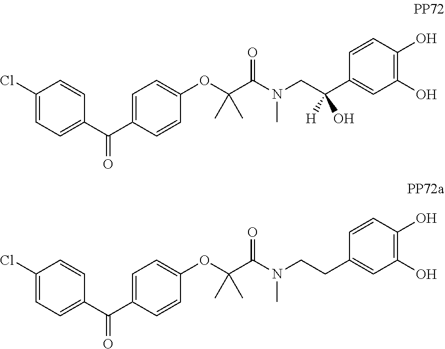 [SMILES: CN(C[C@H](O)c1ccc(O)c(O)c1)C(=O)C(C)(C)Oc1ccc(C(=O)c2ccc(Cl)cc2)cc1 ; CN(CCc1ccc(O)c(O)c1)C(=O)C(C)(C)Oc1ccc(C(=O)c2ccc(Cl)cc2)cc1]

a pharmaceutically acceptable salt thereof, or a derivative thereof.

Aspects of the invention are also directed towards an anti cancer compound comprising the following formula:

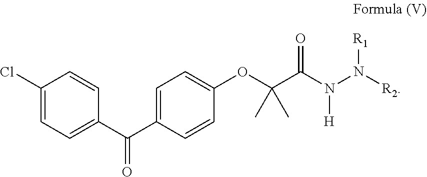

For example, R₁ can be H, CH₃, or (CH₂)₅; R₂ can be H, CH₃, (CH₂)₅.

Non-liming examples of anti cancer compounds of Formula (V) can be found in Example 4. For example, the anti cancer compound can be any of the following structures:

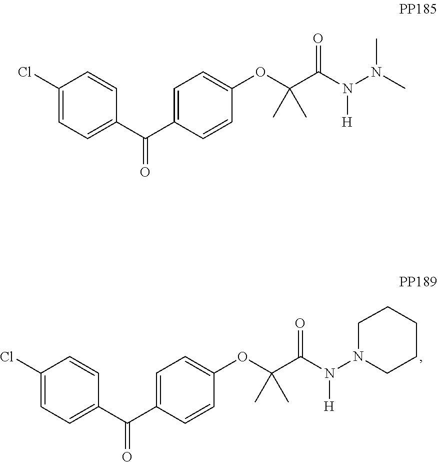 [SMILES: CN(C)NC(=O)C(C)(C)Oc1ccc(C(=O)c2ccc(Cl)cc2)cc1 ; CC(C)(Oc1ccc(C(=O)c2ccc(Cl)cc2)cc1)C(=O)NN1CCCCC1]

pharmaceutically acceptable salt thereof, or a derivative thereof.

Aspects of the invention are also directed towards an anti cancer compound comprising the following formula:

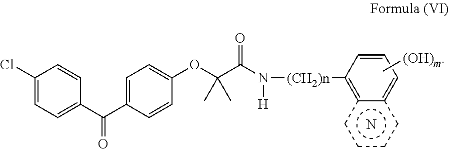

For example, R₁ can be H or CH₃, R₂ can be OH and/or OCH₃, R₃ can be OH and/or OCH₃, R₄ can be OH and/or OCH₃, and/or R₅ can be OH and/or OCH₃.

Non-liming examples of anti cancer compounds of Formula (VI) can be found in Example 4. For example, the anti cancer compound can be any of the following structures:

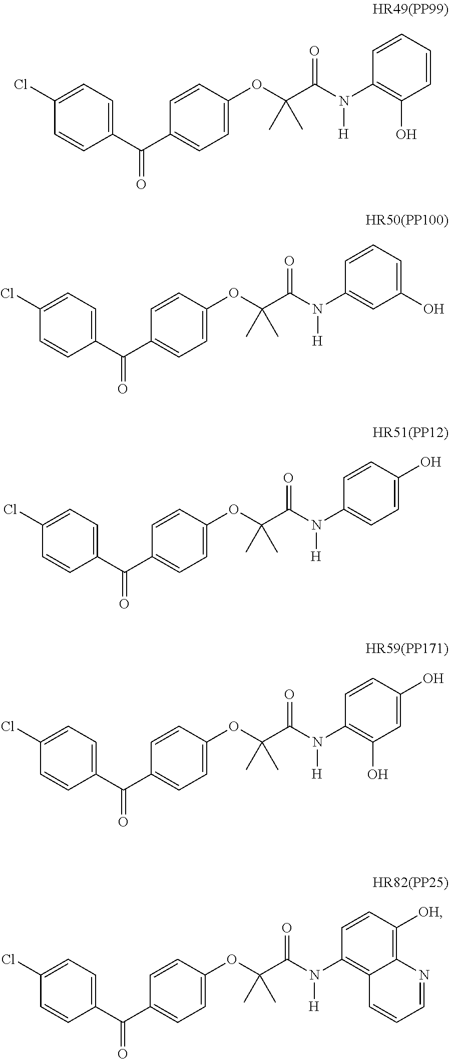 [SMILES: CC(C)(Oc1ccc(C(=O)c2ccc(Cl)cc2)cc1)C(=O)Nc1ccccc1O ; CC(C)(Oc1ccc(C(=O)c2ccc(Cl)cc2)cc1)C(=O)Nc1cccc(O)c1 ; CC(C)(Oc1ccc(C(=O)c2ccc(Cl)cc2)cc1)C(=O)Nc1ccc(O)cc1 ; CC(C)(Oc1ccc(C(=O)c2ccc(Cl)cc2)cc1)C(=O)Nc1ccc(O)cc1O ; CC(C)(Oc1ccc(C(=O)c2ccc(Cl)cc2)cc1)C(=O)Nc1ccc(O)c2ncccc12]

a pharmaceutically acceptable salt thereof, or a derivative thereof.

Aspects of the invention are also directed towards an anti cancer compound comprising the following formula:

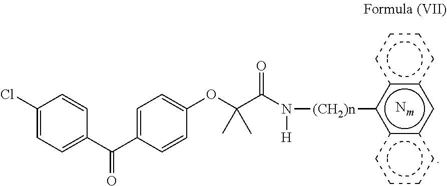

For example, n can be 0, 1 or 2; and wherein m can be 1 or 2.

Non-liming examples of anti cancer compounds of Formula (VII) can be found in Example 4. For example, the anti cancer compound can be any of the following structures:

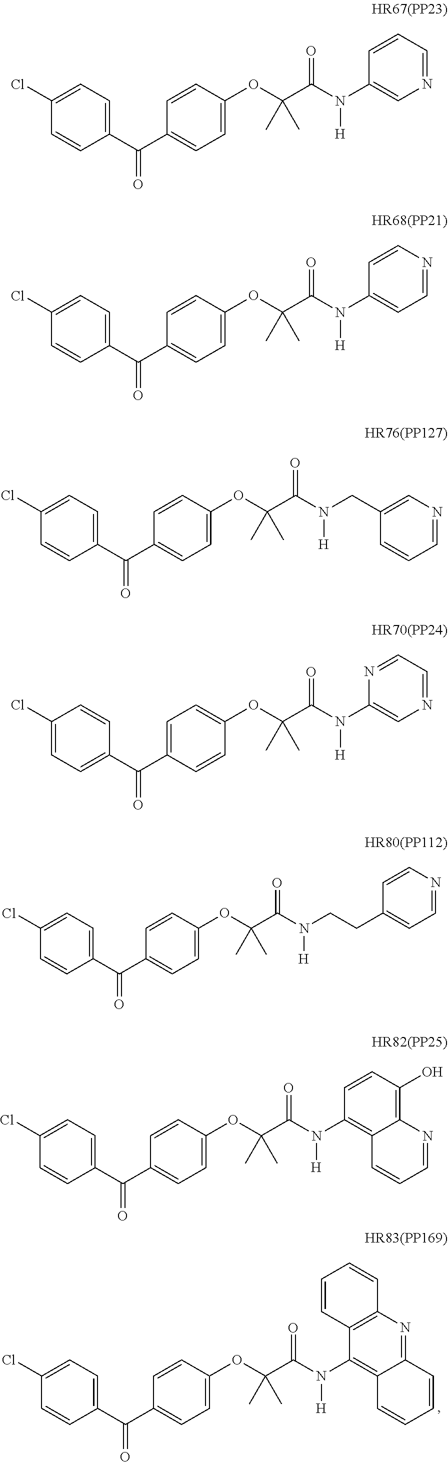

a pharmaceutically acceptable salt thereof, or a derivative thereof.

Aspects of the invention are also directed towards an anti cancer compound comprising the following formula:

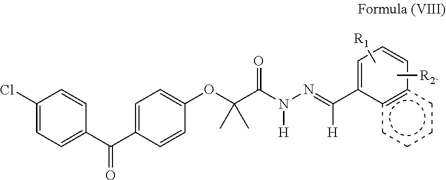

For example, R₁ is H, OH, Cl, Br, NO₂, and wherein R₂ is H, OH, Cl, Br, NO₂.

Non-liming examples of anti cancer compounds of Formula (VIII) can be found in Example 4. For example, the anti cancer compound can be any of the following structures:

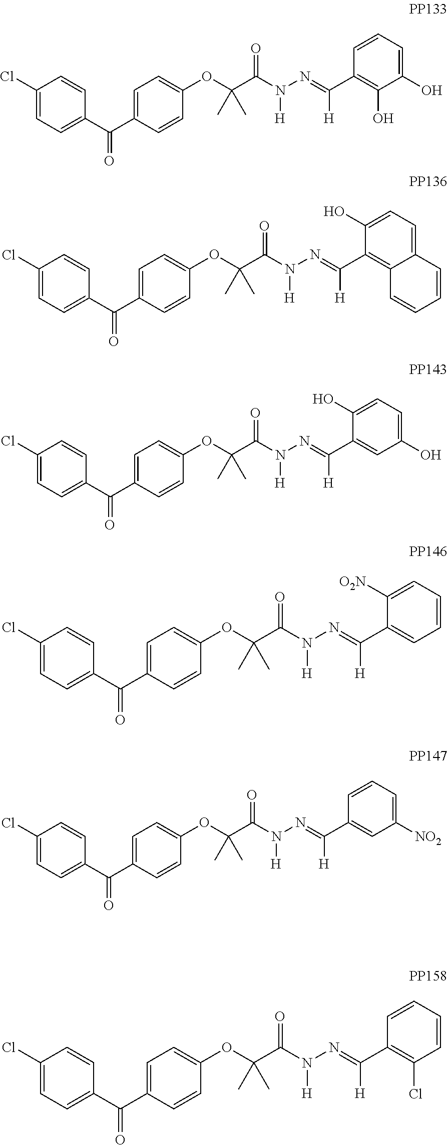 [SMILES: CC(C)(Oc1ccc(C(=O)c2ccc(Cl)cc2)cc1)C(=O)N/N=C/c1cccc(O)c1O ; CC(C)(Oc1ccc(C(=O)c2ccc(Cl)cc2)cc1)C(=O)N/N=C/c1c(O)ccc2ccccc12 ; CC(C)(Oc1ccc(C(=O)c2ccc(Cl)cc2)cc1)C(=O)N/N=C/c1cc(O)ccc1O ; CC(C)(Oc1ccc(C(=O)c2ccc(Cl)cc2)cc1)C(=O)N/N=C/c1ccccc1[N+](=O)[O-] ; CC(C)(Oc1ccc(C(=O)c2ccc(Cl)cc2)cc1)C(=O)N/N=C/c1cccc([N+](=O)[O-])c1 ; CC(C)(Oc1ccc(C(=O)c2ccc(Cl)cc2)cc1)C(=O)N/N=C/c1ccccc1Cl]

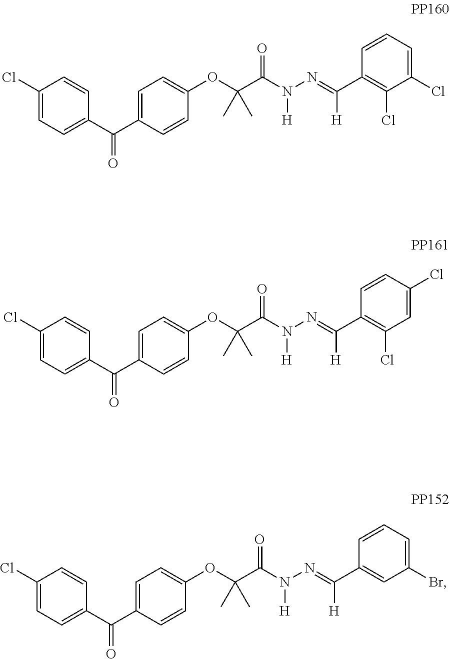 [SMILES: CC(C)(Oc1ccc(C(=O)c2ccc(Cl)cc2)cc1)C(=O)N/N=C/c1cccc(Cl)c1Cl ; CC(C)(Oc1ccc(C(=O)c2ccc(Cl)cc2)cc1)C(=O)N/N=C/c1ccc(Cl)cc1Cl ; CC(C)(Oc1ccc(C(=O)c2ccc(Cl)cc2)cc1)C(=O)N/N=C/c1cccc(Br)c1]

a pharmaceutically acceptable salt thereof, or a derivative thereof.

Aspects of the invention are also directed towards an anti cancer compound comprising the following formula:

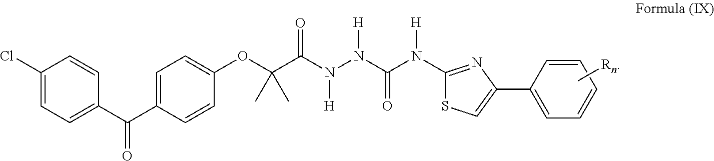

For example, R can be H, Cl, or Br; and wherein n can be 0, 1, 2, or 3.

Non-liming examples of anti cancer compounds of Formula (IX) can be found in Example 4. For example, the anti cancer compound can be any of the following structures:

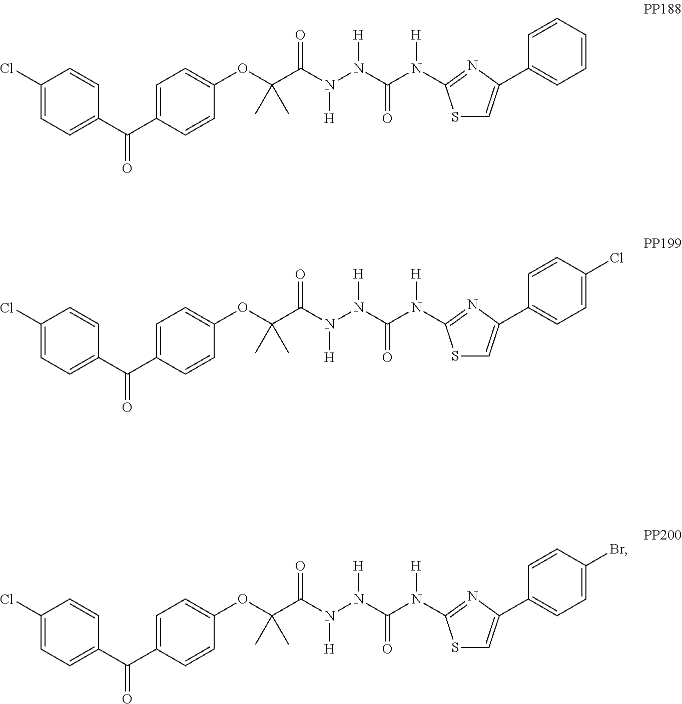 [SMILES: CC(C)(Oc1ccc(C(=O)c2ccc(Cl)cc2)cc1)C(=O)NNC(=O)Nc1nc(-c2ccccc2)cs1 ; CC(C)(Oc1ccc(C(=O)c2ccc(Cl)cc2)cc1)C(=O)NNC(=O)Nc1nc(-c2ccc(Cl)cc2)cs1 ; CC(C)(Oc1ccc(C(=O)c2ccc(Cl)cc2)cc1)C(=O)NNC(=O)Nc1nc(-c2ccc(Br)cc2)cs1]

Aspects of the invention are also directed towards an anti cancer compound comprising the following formula:

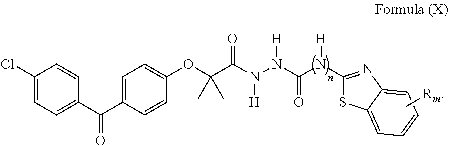

a pharmaceutically acceptable salt thereof, or a derivative thereof.

For example, R can be H, Cl, or Br; wherein n can be 0 or 1; wherein m can be 1, 2, or 3.

Non-liming examples of anti cancer compounds of Formula (X) can be found in Example 4. For example, the anti cancer compound can be any of the following structures:

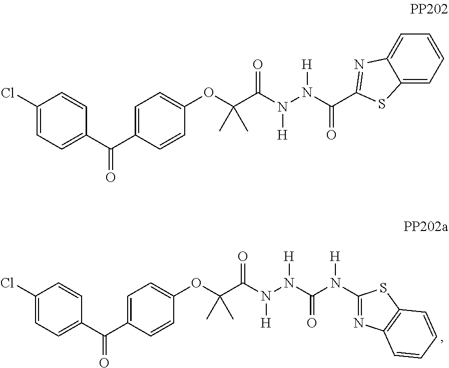 [SMILES: CC(C)(Oc1ccc(C(=O)c2ccc(Cl)cc2)cc1)C(=O)NNC(=O)c1nc2ccccc2s1 ; CC(C)(Oc1ccc(C(=O)c2ccc(Cl)cc2)cc1)C(=O)NNC(=O)Nc1nc2ccccc2s1]

a pharmaceutically acceptable salt thereof, or a derivative thereof.

Aspects of the invention are further directed towards pharmaceutical compositions comprising an anticancer compound as described herein. A “pharmaceutical composition” can refer to preparation of a compound as described herein with other chemical components such as physiologically suitable carriers and/or excipients. The purpose of a pharmaceutical composition is to facilitate administration of a compound to an organism. For example, a pharmaceutical composition can comprise a compound of formula (I) and a pharmaceutically acceptable carrier. For example, a pharmaceutical composition can comprise a compound of formula (II) and a pharmaceutically acceptable carrier. For example, a pharmaceutical composition can comprise a compound of formula (III) and a pharmaceutically acceptable carrier. For example, a pharmaceutical composition can comprise a compound of formula (IV) and a pharmaceutically acceptable carrier. For example, a pharmaceutical composition can comprise a compound of formula (V) and a pharmaceutically acceptable carrier. For example, a pharmaceutical composition can comprise a compound of formula (VI) and a pharmaceutically acceptable carrier. For example, a pharmaceutical composition can comprise a compound of formula (VII) and a pharmaceutically acceptable carrier. For example, a pharmaceutical composition can comprise a compound of formula (VIII) and a pharmaceutically acceptable carrier. For example, a pharmaceutical composition can comprise a compound of formula (IX) and a pharmaceutically acceptable carrier. For example, a pharmaceutical composition can comprise a compound of formula (X) and a pharmaceutically acceptable carrier.

The terms “physiologically acceptable carrier” and “pharmaceutically acceptable carrier” which may be interchangeably used can refer to a carrier or a diluent that does not cause significant irritation to an organism and does not abrogate the biological activity and properties of the administered chelator.

The term “excipient” refers to an inert substance added to a pharmaceutical composition to further facilitate administration of a compound. Examples, without limitation, of excipients include various sugars and types of starch, cellulose derivatives, gelatin, vegetable oils and polyethylene glycols.

According to the invention, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

Non-limiting examples of pharmaceutically acceptable carriers comprise solid or liquid fillers, diluents, and encapsulating substances. Including but not limited to lactose, dextrose, sucrose, sorbitol, mannitol, xylitol, erythritol, maltitol, starches, gum acacia, alginate, gelatin, calcium phosphate, calcium silicate, cellulose, methyl cellulose, microcrystalline cellulose, polyvinylpyrrolidone, water, methyl benzoate, propyl benzoate, talc, magnesium stearate, and mineral oil.

A pharmaceutical composition of the invention can be formulated to be compatible with its intended route of administration. Techniques for formulation and administration of drugs may be found in “Remington's Pharmaceutical Sciences,” Mack Publishing Co., Easton, Pa., latest edition, which is incorporated herein by reference in its entirety. Pharmaceutical compositions of the present invention may be manufactured by processes well known in the art, e.g., by means of conventional mixing, dissolving, granulating, dragee-making, levigating, emulsifying, encapsulating, entrapping or lyophilizing processes

Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the compound in the required amount in an appropriate solvent with one or a combination of ingredients enumerated herein, as required, followed by filtered sterilization. Generally, dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Embodiments of the invention can be provided as a pharmaceutically acceptable salt. The term “pharmaceutically acceptable” can refer to salts or chelating agents are acceptable from a toxicity viewpoint. The term “pharmaceutically acceptable salt” can refer to refer to ammonium salts, alkali metal salts such as potassium and sodium (including mono, di- and tri-sodium) salts (which are preferred), alkaline earth metal salts such as calcium and magnesium salts, salts with organic bases such as dicyclohexylamine salts, N-methyl-D-glucamine, and salts with amino acids such as arginine, lysine, and so forth.

Methods of Synthesis

Embodiments of the invention comprise synthetic schemes and methods to produce, make, or manufacture anticancer compounds described herein.

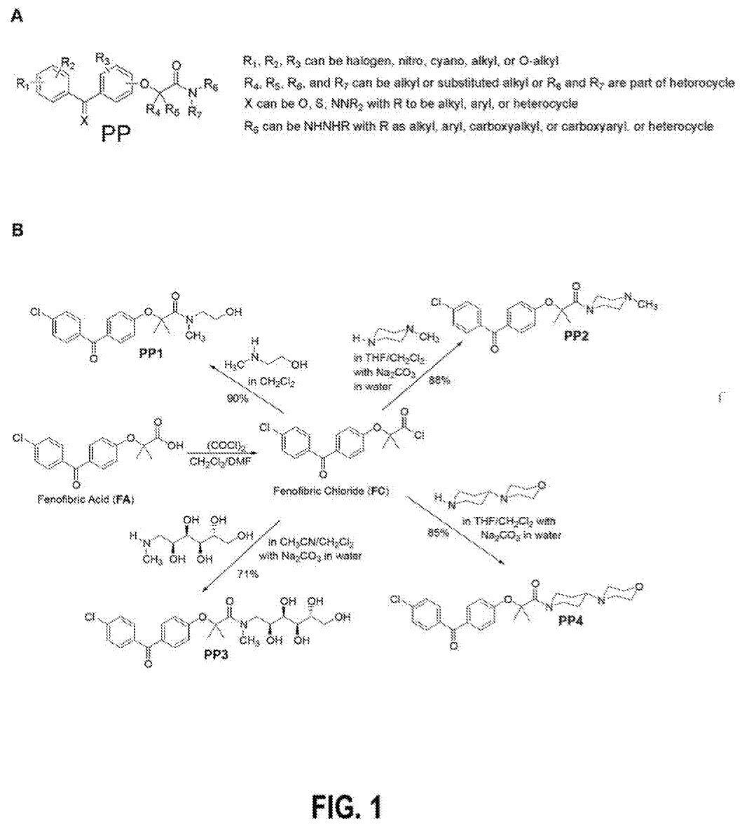

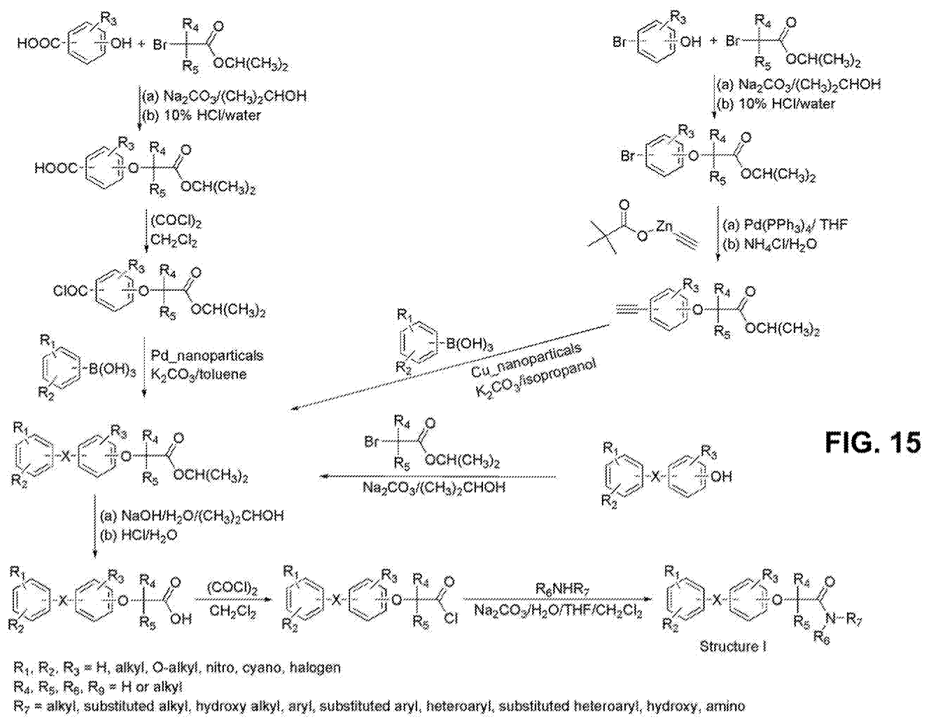

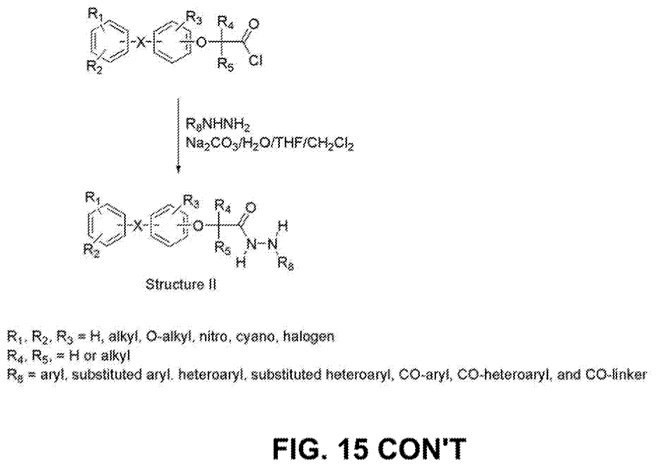

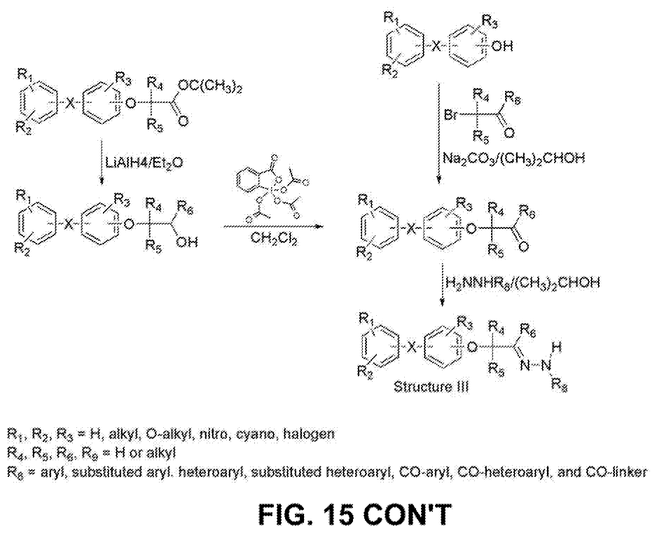

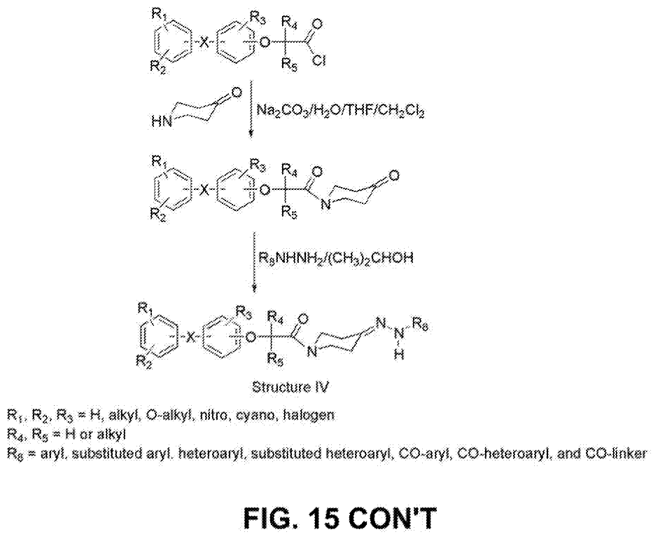

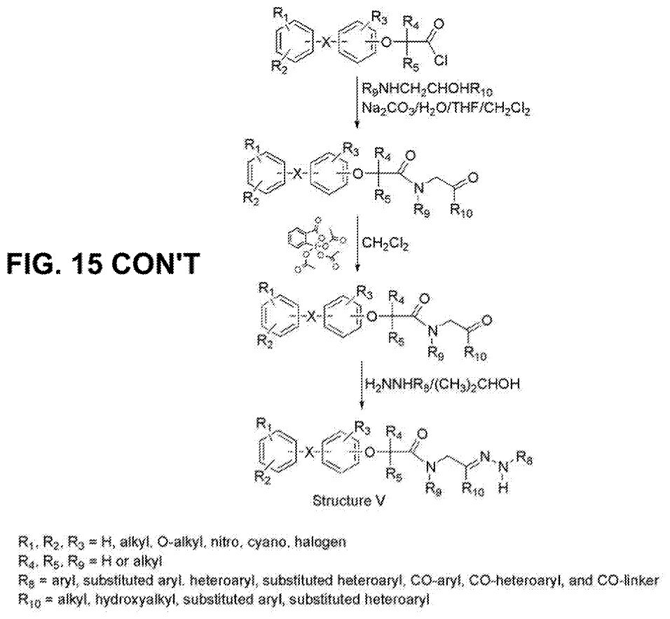

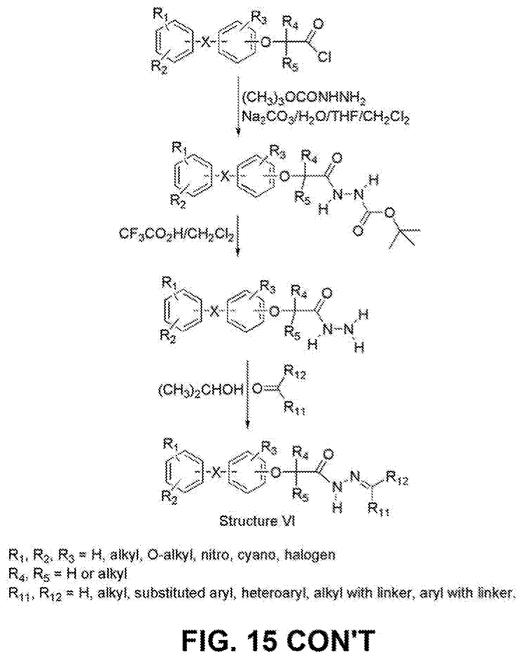

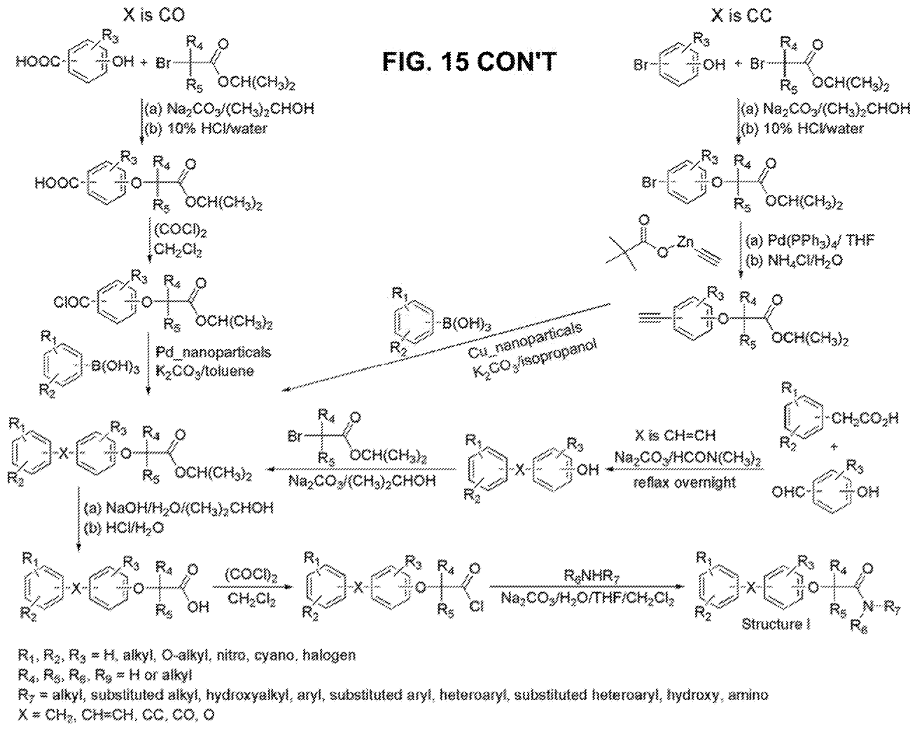

Exemplary synthetic schemes can be found in, for example, FIG. 1, FIG. 11, and FIG. 15.

For example, embodiments are directed towards a method of synthesizing 2-[4-(4-chlorobenzoyl)phenoxy]-N-(2-hydroxyethyl)-N,2-dimethylpropanamide (PP1), the method comprising: Dichloromethane (20 ml) suspension of fenofibric acid (318.75 mg; 1 mmol) and oxalyl chloride (0.25 mL; 380.1 mg; 3 mmol), and one drop od N,N-dimethylformamide was stirred at room temperature for 5 hours. Solvent was evaporated under reduced pressure. White solid residue was resolved in in dichloromethane (10 ml) and again evaporated to the solid residue. This solid residue was dissolved in dichloromethane (20 ml) and at room temperature with stirring dichloromethane (10 ml) solution of 2-(methylamino)ethanol (0.24 ml; 225 mg; 3 mmol) was gradually added. Reaction mixture was stirred at room temperature. Dichloromethane reaction mixture was washed with water (3×15 ml), 5% hydrochloric acid (3×15 ml), water (3×15 ml), 10% sodium carbonate (3×15 ml), and finally with water (3×15 ml) and dried over anhydrous sodium carbonate. Solvent was evaporated under reduced pressure to result in viscous pale-yellow liquid (390 mg). Product was crystalized from dichloromethane/hexane (30 ml; 1:4) at room temperature by slow solvent evaporation to ⅕ original volume and formed crystals were washed with ice cold hexane. Isolated yield 340 mg (90%).

As another example, embodiments are directed towards a method of synthesizing 2-[4-(4-chlorobenzoyl)phenoxy]-N-(2-hydroxyethyl)-N,2-dimethylpropanamide (PP1), the method comprising: Dichloromethane (100 ml) solution of fenofibric acid (637 mg; 2 mmol), 1-ethyl-3-(3-dimethylaminopropyl)carbodiimide, hydrochloride (EDC, 576; 3 mmol), and 2-(methylamino)ethanol (600 mg; 8 mmol) was stirred at room temperature overnight. Dichloromethane solution was washed with 5% hydrochloric acid (5×20 ml), water (5×20 ml), 10% sodium carbonate (5×20 ml), water (3×20 ml) and dried over anhydrous sodium carbonate. After solvent evaporation, oily residue was crystalized from dichloromethane/hexane (1:4) to give pure product (640 mg; 85% yield).

As yet another example, embodiments are directed towards a method of synthesizing 2-[4-(4-chlorobenzoyl)phenoxy]-2-methyl-1-(4-methylpiperazin-1-yl)propan-1-one (PP2), the method comprising: Dichloromethane (30 ml) of fenofibric chloride (1 mmol; prepared as described above for PP1 preparation) was slowly added in stirring water (5 ml) solution of sodium carbonate (216 mg; 2 mmol) with tetrahydrofuran (10 ml) and of 1-methylpiperazine (0.13 ml; 120 mg; 1.2 mmol). Resulting reaction mixture was stirred at room temperature for one hour. Additional water (30 ml) was added and organic layer was separated, washed with water (3×10 ml), 5% hydrochloric acid (3×10 ml), 10% sodium carbonate (3×10 ml), water and dried over anhydrous sodium carbonate. After evaporation oily residue was crystalized from dichloromethane hexane to give 350 mg (88%) of pure product.

As still another example, embodiments are directed towards a method of synthesizing 2-[4-(4-chlorobenzoyl)phenoxy]-N,2-dimethyl-N-[(2S,3R,4R,5R)-2,3,4,5,6-pentahydroxyhexyl]propenamide (PP3), the method comprising: Fenofibric acid chloride prepared from fenofibric acid (318.75 mg; 1 mmol) and oxalyl chloride (0.25 mL; 380.1 mg; 3 mmol) as described for preparation of PP1 was dissolved in dichloromethane (15 ml) and mixed with acetonitrile (20 ml) and water (10 ml) solution of N-methyl-D-glucamine (196 mg; 1 mmol) and sodium carbonate (212 mg; 2 mmol). Resulting mixture was stirred at room temperature for five minutes and solvent was evaporated under reduce pressure at room temperature. Resulting solid residue was mixed with dichloromethane (50 ml) and water (20 ml). Dichloromethane layer was separated, washed with 10% sodium carbonate (3×10 ml), water (3×10 ml) and dried over anhydrous sodium carbonate. After solvent was evaporated solid residue was washed with hexane (3×5 ml) and dried in vacuum under reduce pressure to give 350 mg (71%) of pure product.

As an example, embodiments are directed towards a method of synthesizing 2-[4-(4-chlorobenzoyl)phenoxy]-2-methyl-1-[4-(morpholin-4-yl)piperidin-1-yl]propan-1-one (PP4), the method comprising: Fenofibric acid chloride prepared from fenofibric acid (160 mg; 0.5 mmol) and oxalyl chloride (0.25 mL; 380.1 mg; 3 mmol) as described for preparation of PP1 was dissolved in dichloromethane (30 ml) and mixed with tetrahydrofuran (10 ml) solution of 4-morpholinopiperidine (100 mg; 0.6 mol), and water (10 ml) solution of sodium carbonate (106 mg; 1 mmol). Resulting mixture was stirred at room temperature for one hour. Water (20 ml) was added and organic layer was separated and extensively washed with 5% hydrochloric acid (5×20 ml), water (3×10 ml), 10% sodium carbonate (5×20 ml) and again with water (3×10 ml). After drying over anhydrous sodium carbonate solvent was evaporated to give pure product (200 mg; 85%) as oil that crystallized by standing at room temperature overnight. If necessary, the product can be further purified by crystallization from hexane or by silica gel chromatography with ethyl acetate-ethanol (5:1).

As an example, embodiments are directed towards a method of synthesizing 4-(1-{2-[4-(4-chlorobenzoyl)phenoxy]-2-methylpropanoyl}piperidin-4-yl)morpholin-4-iumchloride (PP4HCl)—A mixture of concentrated hydrochloric acid (2 ml) and PP4 (94 mg; 0.2 mmol) was sonicated at room temperature for half an hour. Clear solution was evaporated to dryness at room temperature under reduced pressure. Residue was dissolved in dry ether (20 ml) and clear solution was left at room temperature for solvent to slowly evaporate. For white crystalline material was separated by filtration and washed with ice-cold hexane to give 80 mg (79%) of pure product.

The skilled artisan will recognize the synthetic schemes and mechanisms can be adapted to provide different embodiments of the invention, derivative compounds, and/or pharmaceutically acceptable salts, such as those described herein.

Methods of Treatment

Aspects of the invention are directed towards methods of treating a subject with a cancer. As used herein, the terms “tumor” and “cancer” can be used interchangeably, and generally refer to a physiological condition characterized by the abnormal and/or unregulated growth, proliferation or multiplication of cells.

The terms “treat,” “treating” or “treatment” can refer to the lessening of severity of a tumor or cancer, delay in onset of a tumor or cancer, slowing the growth of a tumor or cancer, slowing metastasis of cells of a tumor or cancer, shortening of duration of a tumor or cancer, arresting the development of a tumor or cancer, causing regression of a tumor or cancer, relieving a condition caused by a tumor or cancer, or stopping symptoms which result from a tumor or cancer. The terms “treat,” “treating” or “treatment”, can include, but are not limited to, prophylactic and/or therapeutic treatments. For example, the invention is directed towards methods of reducing cell viability and/or promoting apoptosis of cancer cells by administering to a subject in need thereof a therapeutically effective amount of an anticancer compound or composition. The invention is also directed towards methods of attenuating abnormal cell proliferation, and methods of delaying or inhibiting metastatic invasion of a cancer cell.

The approach as described herein (i.e., administration of an anticancer compound or pharmaceutical composition to a subject in need thereof) will provide clinical benefit, defined broadly as any of the following: inhibiting an increase in cell volume, slowing or inhibiting worsening or progression of cancer cell proliferation, reducing primary tumor size, reducing occurrence or size of metastasis, reducing or stopping tumor growth, inhibiting tumor cell division, killing a tumor cell, sensitizing a tumor cell to a drug, radiation, or chemical, inducing apoptosis in a tumor cell, reducing or eliminating tumor recurrence.

In embodiments, the method comprises administering to the subject a therapeutically effective amount of an anticancer compound or pharmaceutical composition described herein. The term “administer” or “administration” can refer to introducing an anticancer compound or pharmaceutical composition into a subject. In general, any route of administration can be utilized. Non-limiting examples of routes of administration comprise parenteral (e.g., intravenous), intraperitoneal, oral, topical, subcutaneous, peritoneal, intraarterial, inhalation, vaginal, rectal, nasal, introduction into the cerebrospinal fluid, or instillation into body compartments. In some embodiments, administration is intraperitoneal. Additionally, or alternatively, in some embodiments, administration is parenteral. In some embodiments, administration is intravenous. In other embodiments, administration is orally.

An anticancer compound or pharmaceutical composition can be administered to a subject in need thereof one time (e.g., as a single injection or deposition). Alternatively, administration can be once or twice daily to a subject in need thereof for a period of from about 2 to about 28 days, or from about 7 to about 10 days, or from about 7 to about 15 days. It can also be administered once or twice daily to a subject for a period of 1, 2, 3, 4, 5, 6, 7, 8, 9, 10, 11, 12 times per year, or a combination thereof. It can also be administered once or twice daily to a subject for a period of years or until the death of the subject, such as can be the case for a subject suffering from pancreatic cancer.

An anticancer compound or pharmaceutical composition can be incorporated into a delivery system for administration to a subject. The term “delivery system” can refer to any form of a composition, such as a solid, semi-solid, or liquid, having an anticancer compound or such pharmaceutical composition incorporated therein which can deliver the anticancer compound to/into a cell, such as a cancer cell. The delivery system can be a biodegradable delivery system. The pharmaceutical composition can be designed to have a desired release rate of the anticancer compound incorporated therein. The delivery system comprising the anticancer compound can be administered to a subject as described herein.

In embodiments, the delivery system comprises a nanoparticle. The term “nanoparticle” can refer to a carrier structure which is biocompatible with and sufficiently resistant to chemical and/or physical destruction by the environment of use such that a sufficient amount of the nanoparticles remain substantially intact after injection into the blood stream, or given intraperitoneally or orally, so as to be able to reach a cancer cell. Nanoparticles can be solid colloidal particles ranging in size from 1 to 1000 nm. Drugs, such as an anticancer compound described herein, or other relevant materials (e.g., those used for diagnostic purposes in nuclear medicine or in radiation therapy) can be dissolved within the nanoparticles, entrapped, encapsulated and/or adsorbed or attached.

Nanoparticles can be made from a broad number of materials including acrylates, methacrylates, methylmethacrylates, cyanoacrylates, acrylamides, polyacetates, polyglycolates, polyanhydrides, polyorthoesters, gelatin, polysaccharides, albumin, polystyrenes, polyvinyls, polyacroleines, polyglutataldehydes, and derivatives, copolymers, and derivatives thereof. Monomer materials particularly suitable to fabricate biodegradable nanoparticles by emulsion polymerization in a continuous aqueous phase include methylmethacrylates, polyalkycyanoacrylates, hydroxyethylmethacrylates, methacrylate acid, ethylene glycol dimethacrylate, acrylamide, N,N′-bismethyleneacrylamide and 2-dimethylaminoethyl methacrylate. Other nanoparticles are made by different techniques from N, N-L-lysinediylterephthalate, alkylcyanoacrylate, polylactic acid, polylactic acid-polyglycolic acid-copolymer, polyanhydrates, polyorthoesters, gelatin, albumin, and desolvated macromolecules or carbohydrates. Further, non-biodegradable materials can be used such as polystyrene, poly (vinylpyridine), polyacroleine and polyglutaraldehyde. A summary of materials and fabrication methods for making nanoparticles has previously been published. See Kreuter, J. (1991) “Nanoparticles-preparation and applications.” In: M. Donbrow (Ed.) “Microcapsules and nanoparticles in medicine and pharmacy.” CRC Press, Boca Raton, Fla., pp. 125-148.

Nanoparticles can be produced by conventional methods, including emulsion polymerization in a continuous aqueous phase, emulsion polymerization in continuous organic phase, interfacial polymerization, solvent deposition, solvent evaporation, dissolvation of an organic polymer solution, cross-linking of water-soluble polymers in emulsion, dissolvation of macromolecules, and carbohydrate cross-linking. These fabrication methods can be performed with a wide range of polymer materials mentioned above.

A “therapeutically effective amount” of an anticancer composition or compound can refer to an amount of an anticancer compound or composition sufficient to provide a benefit in the treatment of cancer, to delay or minimize symptoms associated with cancer, or to cure or ameliorate cancer. In particular, a therapeutically effective amount means an amount of an anticancer compound sufficient to provide a therapeutic benefit in vivo. The term preferably encompasses a non-toxic amount of an anticancer compound that improves overall therapy, reduces or avoids symptoms or causes of disease, or enhances the therapeutic efficacy of or synergies with another therapeutic agent.

A therapeutically effective dose of an anticancer compound or composition can depend upon a number of factors known to those of ordinary skill in the art. The dose(s) can vary, for example, depending upon the identity, size, and condition of the subject or sample being treated, further depending upon the route by which the composition is to be administered, if applicable, and the effect which the practitioner desires. It is understood that a medical professional will typically determine the dosage regimen in accordance with a variety of factors. These factors include the cancer and/or tumor from which the subject suffers, the degree of metastasis, as well as the age, weight, sex, diet, and medical condition of the subject.

In some embodiments, the therapeutically effective amount is at least about 0.1 mg/kg body weight, at least about 0.25 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 3500 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight.

Referring to the Examples, anticancer effects and minimal toxicity of compound PP1 were observed in mice bearing intracranial glioblastomas at a doses of 25-75 mg/kg body weight. The exact dosage will be determined by the practitioner, in light of factors related to the patient who requires treatment. Dosage and administration are adjusted to provide sufficient levels of the anticancer compound or to maintain the desired effect. Factors that can be taken into account include the type of subject (i.e., human, dog, or otherwise), severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Described herein are methods of treating a subject afflicted with cancer comprising administering to a subject a therapeutically effective amount of an anticancer compound or composition. The terms “individual”, “patient” and “subject” can be used interchangeably. They refer to a mammal (e.g., a human) which is the object of treatment, or observation. Typical subjects to which the anticancer compound or composition can be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like.

Aspects of the invention are directed towards methods of inhibiting or delaying metastasis of a cancer cell, and/or inhibiting or delaying invasion of a cancer cell. A cancer cell is a cell(s) that divide uncontrollably, forming solid tumors or flooding the blood with abnormal cells. The term “metastasis” refers to the ability of tumor cells to invade host tissues and metastasize to distant, often specific organ sites. As is known, this is the salient feature of lethal tumor growths. Metastasis formation occurs via a complex series of unique interactions between tumor cells and normal host tissues and cells. “Metastasis” is distinguished from invasion, which can refer to the direct migration and penetration by cancer cells into neighboring tissues. In other words, invasion refers to the direct extension and penetration by cancer cells into neighboring tissues. The proliferation of transformed cells and the progressive increase in tumor size eventually leads to a breach in the barriers between tissues, leading to tumor extension into adjacent tissue. Local invasion is also the first stage in the process that leads to the development of secondary tumors or metastases.

Aspects of the invention are also directed towards methods of treating a subject afflicted with a disease, such as cancer or a tumor.

Cancers are classified by the type of cell that the tumor cells resemble and is therefore presumed to be the origin of the tumor. Cancer types include carcinoma (cancers derived from epithelial cells), sarcomas (cancers arising from connective tissue), lymphoma and leukemia (cancers arising from hematopoietic cells), germ cell tumors (cancers derived from pluripotent cells), and blastoma (cancer derived from immature “precursor” cells or embryonic tissue).

Carcinomas refer to malignancies of epithelial or endocrine tissue, and include respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from the cervix, lung, prostate, breast, head and neck, pancreas, colon, liver and ovary. The term also includes carcinosarcomas, e.g., which include malignant tumors composed of carcinomatous and sarcomatous tissues. Adenocarcinoma includes a carcinoma of a glandular tissue, or in which the tumor forms a gland like structure.

Sarcomas comprise cancers arising from connective tissue (i.e. bone, cartilage, fat, nerve), each of which develops from cells originating in mesenchymal cells outside the bone marrow. Exemplary sarcomas include for example, lymphosarcoma, liposarcoma, osteosarcoma, and fibrosarcoma.

Lymphoma and leukemia arise from hematopoietic (blood-forming) cells that leave the marrow and tend to mature in the lymph nodes and blood, respectively. Non-limiting examples include acute leukemia, erythroblastic leukemia and acute megakaryoblastic leukemia, acute promyeloid leukemia (APML), acute myelogenous leukemia (AML) and chronic myelogenous leukemia (CML); lymphoid malignancies include, but are not limited to, acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional malignant lymphomas include, but are not limited to, non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

Germ cell tumors are cancers derived from pluripotent cells, most often presenting in the testicle or the ovary (seminoma and dysgerminoma, respectively).

Blastomas are cancers derived from immature “precursor” cells or embryonic tissue.

The term “solid tumor” refers to hyperplasias, neoplasias or metastases that typically aggregate together and form a mass. Non-limiting examples include visceral tumors such as gastric or colon cancer, hepatomas, venal carcinomas, lung and brain tumors/cancers.

A “liquid tumor” generally refers to neoplasias of the haematopoetic system, such as lymphomas, myelomas and leukemias, or neoplasias that are diffuse in nature, as they do not typically form a solid mass. Non-limiting examples of leukemias include acute and chronic lymphoblastic, myeloblastic and multiple myeloma.

Neoplasms or cancers can affect virtually any cell or tissue type, e.g., carcinoma, sarcoma, melanoma, metastatic disorders or haematopoietic neoplastic disorders. A metastatic tumor can arise from a multitude of primary tumor types, including but not limited to breast, lung, thyroid, head and neck, brain, lymphoid, gastrointestinal (mouth, esophagus, stomach, small intestine, colon, rectum), genito-urinary tract (uterus, ovary, cervix, bladder, testicle, penis, prostate), kidney, pancreas, liver, bone, muscle, skin, etc.

“Glioma” refers to a tumor that arises from glial cells or their precursors of the brain or spinal cord. Gliomas are histologically defined based on whether they exhibit primarily astrocytic or oligodendroglial morphology, and are graded by cellularity, nuclear atypia, necrosis, mitotic figures, and microvascular proliferation-all features associated with biologically aggressive behavior. Astrocytomas are of two main types-high-grade and low-grade. High-grade tumors grow rapidly, are well-vascularized, and can easily spread through the brain. Low-grade astrocytomas are usually localized and grow slowly over a long period of time. High-grade tumors are much more aggressive, require very intensive therapy, and are associated with shorter survival lengths of time than low grade tumors. The majority of astrocytic tumors in children are low-grade, whereas the majority in adults are high-grade. These tumors can occur anywhere in the brain and spinal cord. Some of the more common low-grade astrocytomas are: Juvenile Pilocytic Astrocytoma (JPA), Fibrillary Astrocytoma Pleomorphic Xantroastrocytoma (PXA) and Desembryoplastic Neuroepithelial Tumor (DNET). The two most common high-grade astrocytomas are Anaplastic Astrocytoma (AA) and Glioblastoma Multiforme (GBM).

Embodiments herein can be used to treat ependymomas, for example, or oligodendrogliomas. Ependymomas arise from ependymal cells that line the ventricles of the brain and the center of the spinal cord. Oligodendrogliomas are a type of glioma that are believed to originate from the oligodendrocytes of the brain or from a glial precursor cell.

Kits

The anticancer compounds and pharmaceutical compositions described herein can also be provided in a kit for treating cancer, such as gliomas. In one embodiment, the kit includes (a) a container that contains the anticancer compound or pharmaceutical composition, and optionally (b) informational material for treating a specific type of cancer, such as a glioma or leukemia. The informational material can be descriptive, instructional, marketing or other material that relates to the methods described herein and/or the use of the compound for therapeutic benefit. In an embodiment, the kit includes also includes a second agent for treating a cancer. For example, the kit includes a first container that contains an anticancer compound or pharmaceutical composition, and a second container that includes the second agent.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the compound, e.g., in a suitable dose, dosage form, or mode of administration (e.g., a dose, dosage form, or mode of administration described herein), to treat a subject who has cancer. The information can be provided in a variety of formats, include printed text, computer readable material, video recording, or audio recording, or any information that provides a link or address to substantive material.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Chemically Modified Variants of Fenofibric Acid (PP Compounds) with High Anticancer Efficacy

PP compounds are unique chemical modifications of Fenofibric Acid. When compared to unprocessed fenofibrate, PP compounds show significantly higher anticancer efficacy in vitro, are much more stable in blood and tissues, and penetrate blood-brain tumor barrier. As described herein, administration of PP compounds, such as oral administration of PP compounds, can attenuate primary tumor growth and/or metastatic invasion.

According to our previous work, a common lipid lowering drug, fenofibrate, targets energy metabolism of tumor cells, including glioblastoma, triggering severe metabolic deficit, which is followed extensive tumor cell death. Importantly, fenofibrate is practically harmless to normal differentiated cells including cells from the Central Nervous System (CNS). However, fenofibrate does not cross blood brain tumor barrier, and is highly unstable in blood and tissue environment. Therefore, its anticancer efficacy in vivo is limited.

Described herein are new chemical modifications of fenofibric acid generated a series of compounds (can be referred to as PP compounds), which in comparison to unprocessed fenofibrate demonstrate a superior anticancer efficacy in vitro. The compounds are significantly more stable, reaching therapeutically relevant concentrations of the drug in different tissues, and importantly penetrate blood brain tumor barrier, which is critical for developing new PP-based anti-glioblastoma therapy.

Additional evidence supporting overall anti-cancer efficacy of the PP compounds will be demonstrated by more experimental data in tumor animal models. Without wishing to be bound by theory, for example, such experiments will demonstrate that PP compounds are highly effective against brain tumors, including glioblastoma, and different solid and blood cancers.

### Example 2

Chemically Modified Variants of Fenofibrate with High Anti-Glioblastoma Efficacy

Abbreviations: 13C-NMR, carbon-13 nuclear magnetic resonance; 1H-NMR, proton nuclear magnetic resonance; ATP, adenosine triphosphate; BBTB, blood brain tumor barrier; CDCl3, deuterated chloroform; C Log P, Calculated Partitioning; DAD, Diode Array Detector; DMEM, Dulbecco's modified Eagle's medium; DMSO, dimethyl sulfoxide; ECAR, extracellular acidification rate; EDC, 1-Ethyl-3-(3-dimethylaminopropyl) carbodiimide; EDTA, ethylenediaminetetraacetic acid; ETC, Electron Transport Chain; FA, fenofibric acid; FBS, fetal bovine serum; FC, fenofibric chloride; FCCP, carbonylcyanide-p-trifluoromethoxyphenylhydrazone; FF, fenofibrate; GBM, glioblastoma multiforme; HBA, hydrogen bond acceptor; HBD, hydrogen bond donor; HPLC, high performance liquid chromatography; MP, Molecular Polarizability; MSA, Molecular Surface Area; MW, molecular weight; NAD, nicotinamide adenine dinucleotide; OCR, oxygen consumption rate; PBS, phosphate buffered saline; PPARa, peroxisome proliferator activated receptor alpha; PPRE, PPAR responsive element; PSA, Polar Surface Area; siRNA, small interfering RNA.

Abstract

Anticancer effects of a common lipid-lowering drug, fenofibrate (FF) have been described in the literature for a quite some time, however, FF has not been used as a direct anticancer therapy. We have previously reported that FF in its unprocessed form (ester) accumulates in mitochondria, inhibits mitochondrial respiration, triggering a severe energy deficit and extensive glioblastoma cell death. However, FF does not cross blood brain barrier, and is quickly processed by blood and tissue esterases to form PPARα agonist, fenofibric acid (FA). In comparison to unprocessed FF, FA is much less effective in triggering cancer cell death.

To address these issues, we have made several chemical modifications in FF structure to increase its stability, water solubility, tissue penetration, and ultimately, anticancer potential. As exemplary embodiments, our data show that four new compounds designated as PP1, PP2, PP3 and PP4 (see Table 1) have improved anticancer activity when compared to FF. Like FF, they block mitochondrial respiration and trigger massive glioblastoma cell death in vitro. In addition, PP1 has improved water solubility, and is much more stable when exposed to human blood in comparison to FF. In comparison to controls, mice bearing large intracranial tumors demonstrated extensive necrotic areas within the tumor mass following two weeks of daily oral administration of PP1. We also demonstrated that the treated mice accumulated PP1 in different tissues, including intracranial tumors, and survived the treatment without major signs of distress.

Introduction

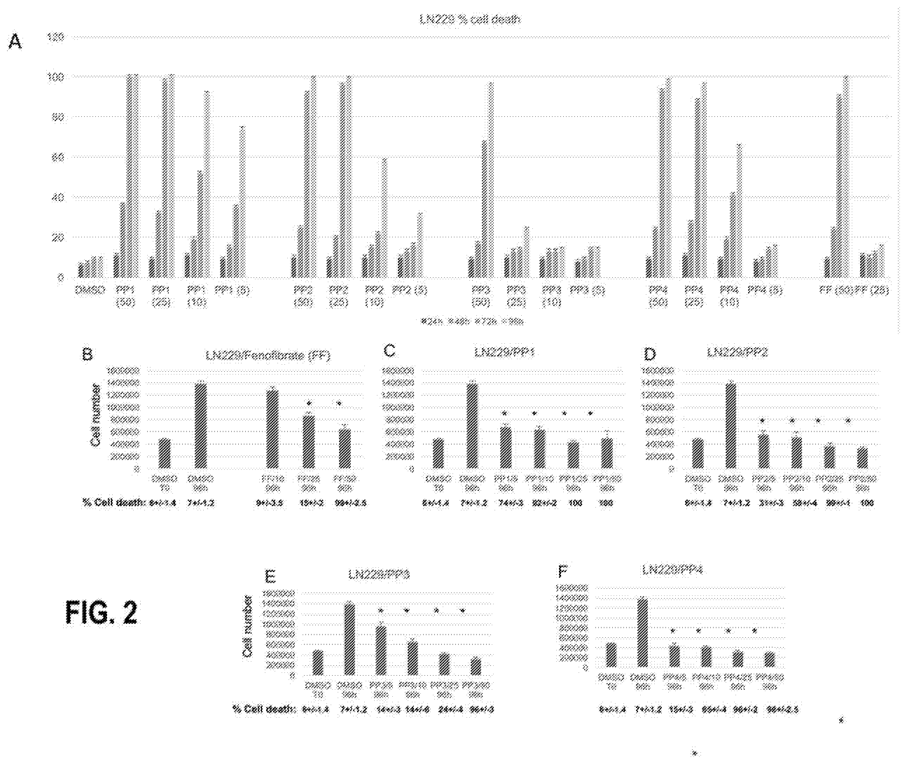

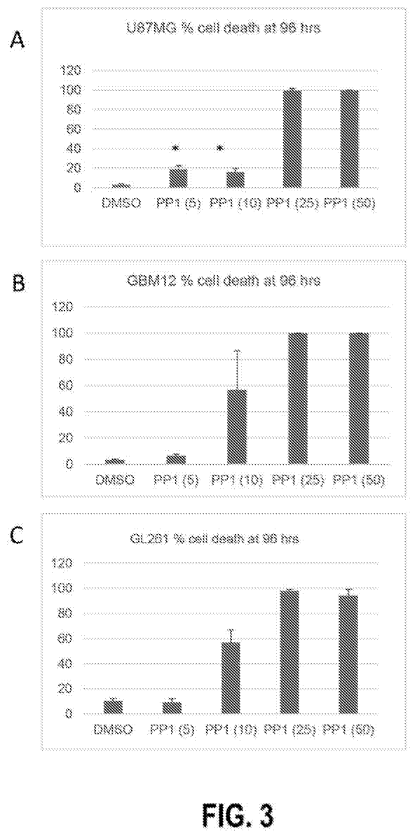

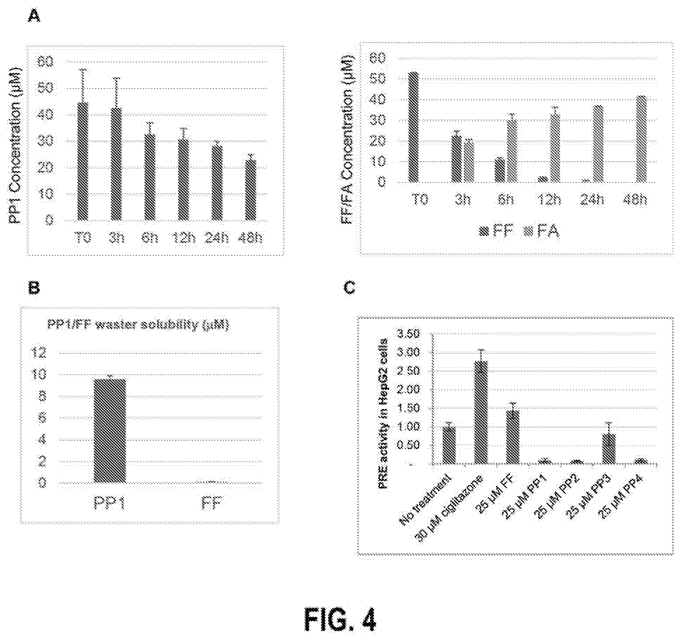

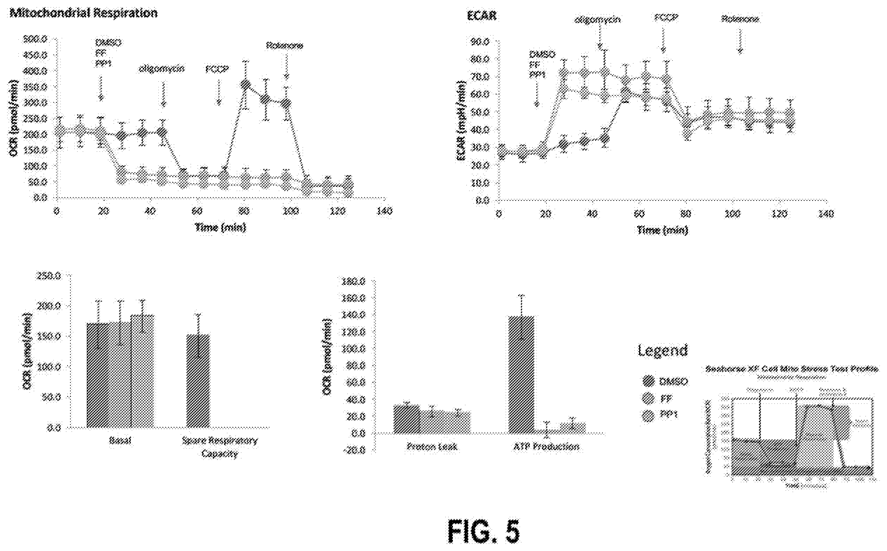

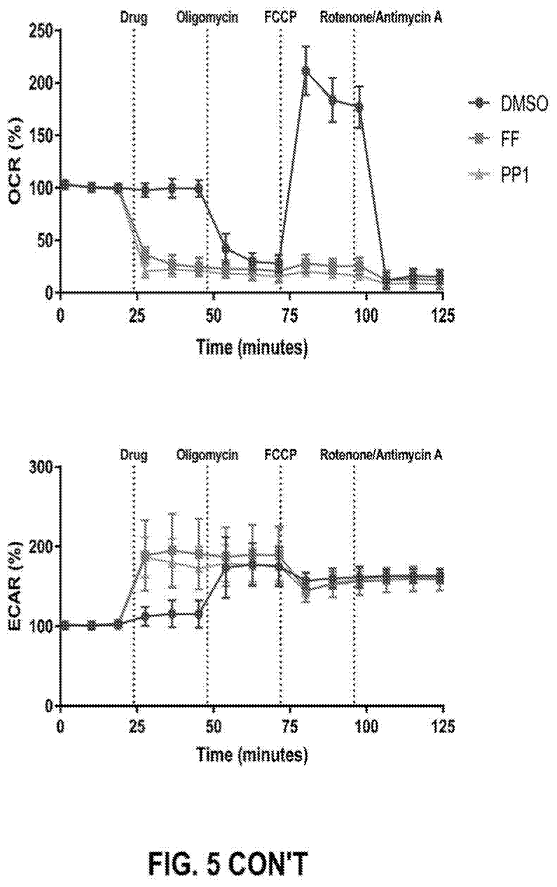

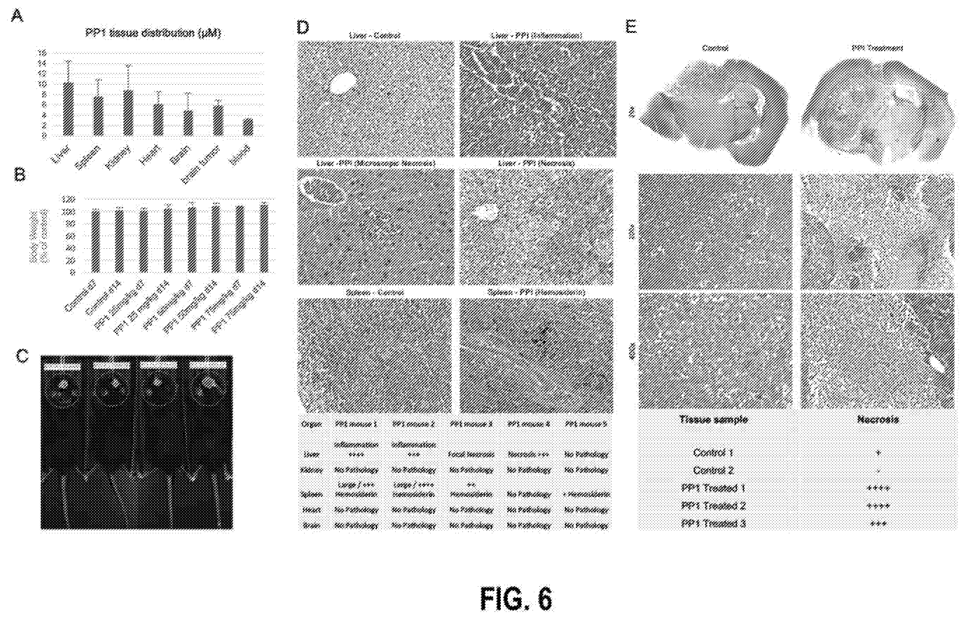

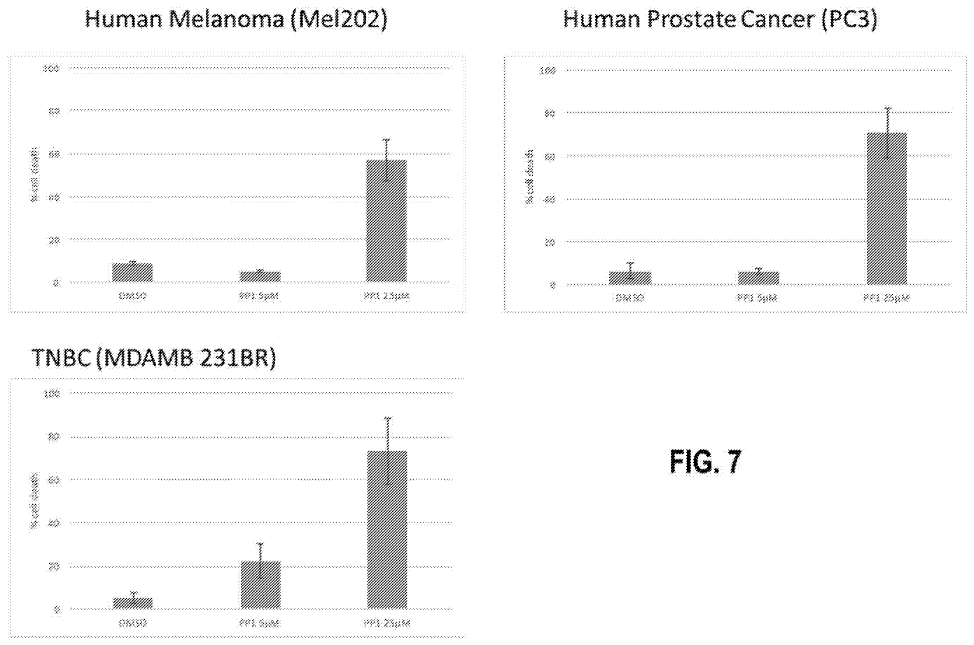

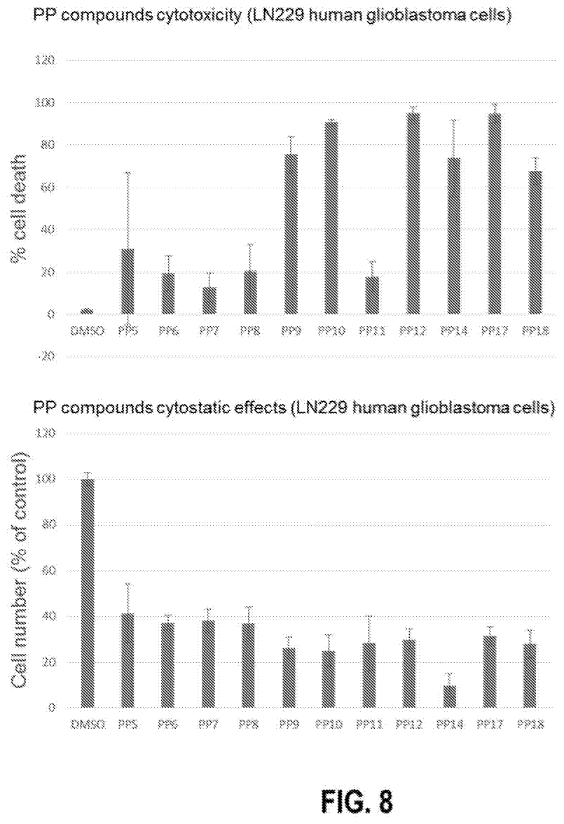

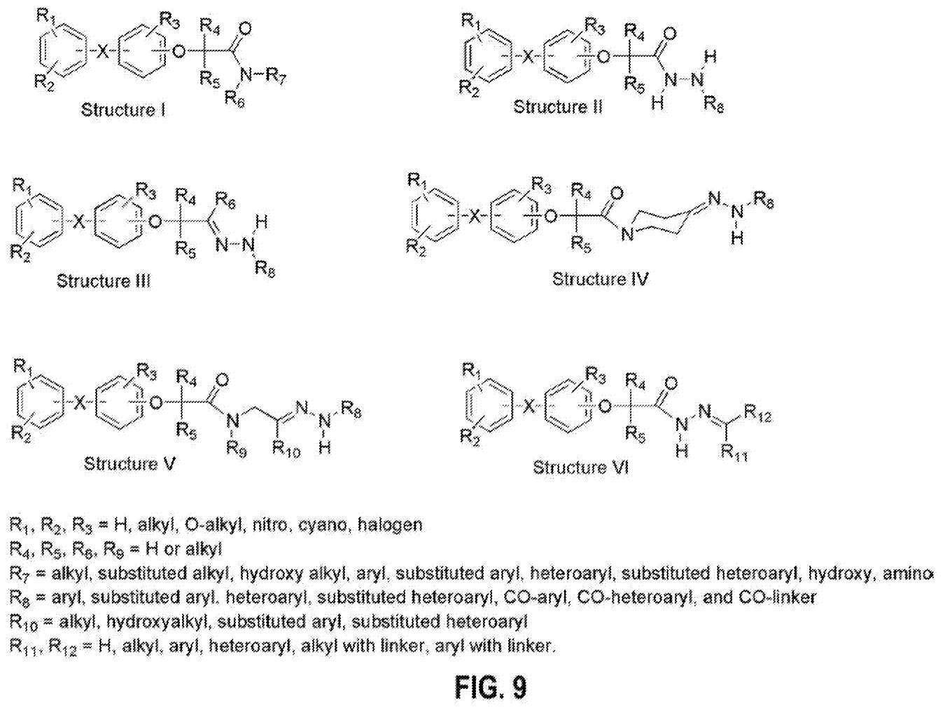

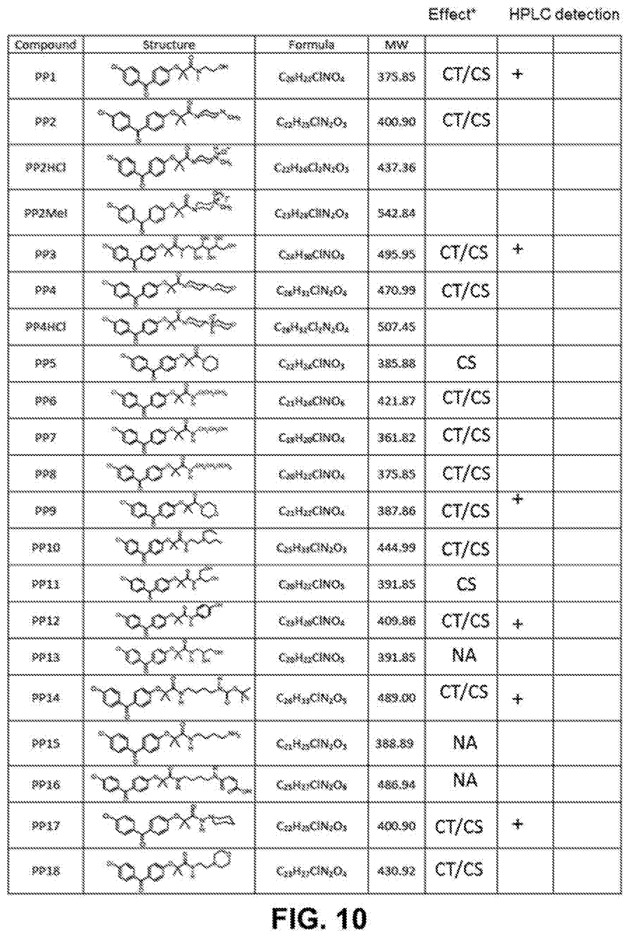

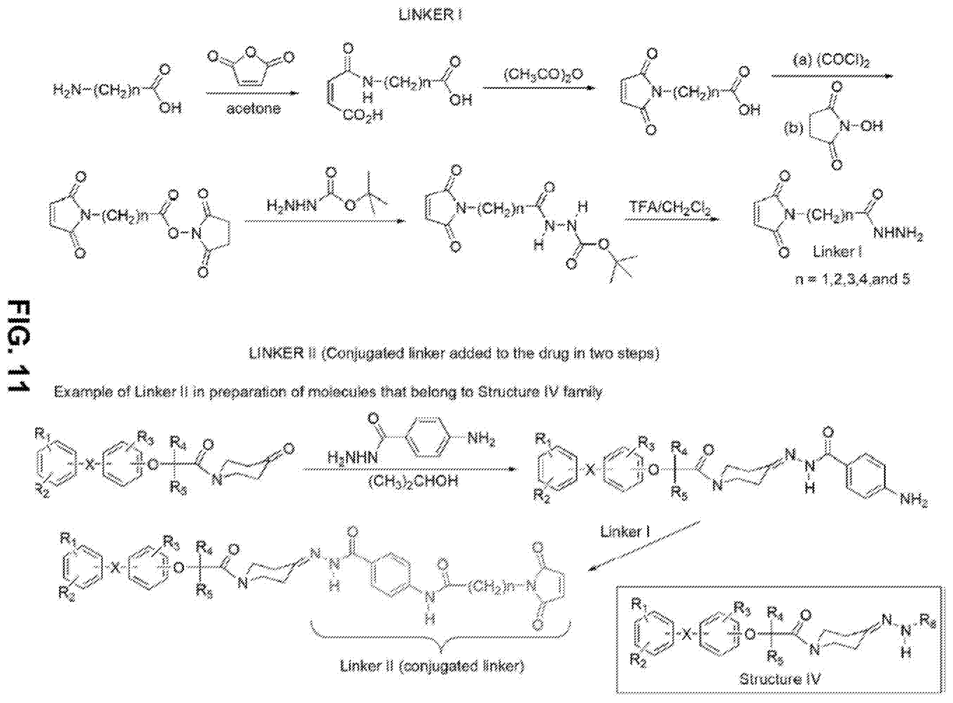

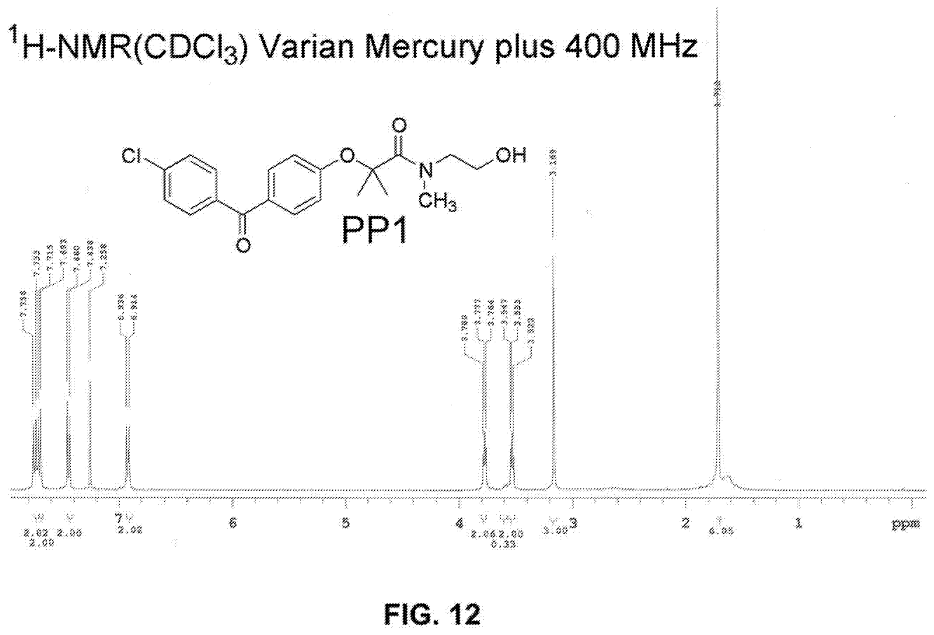

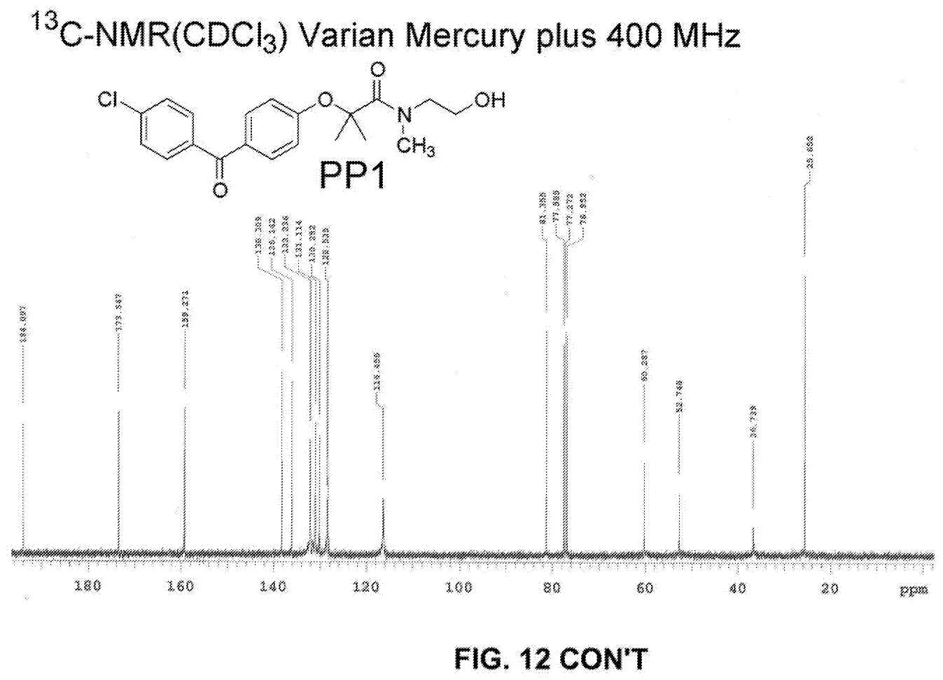

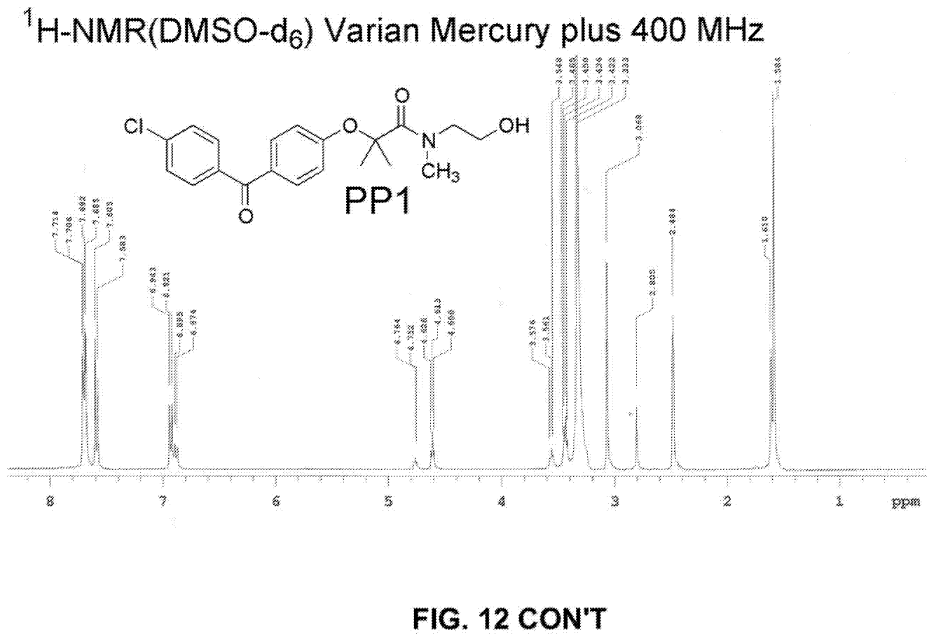

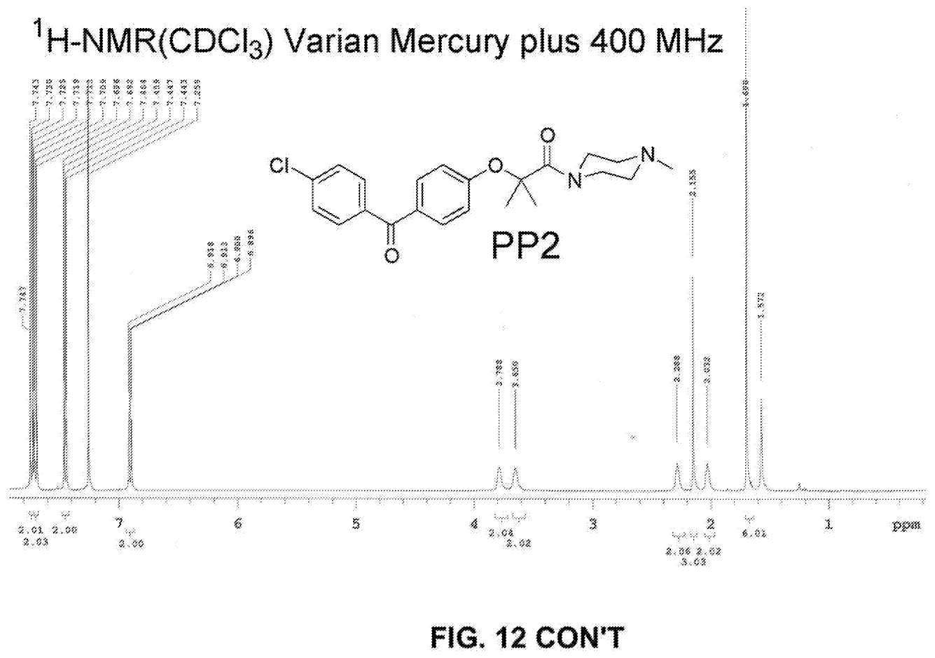

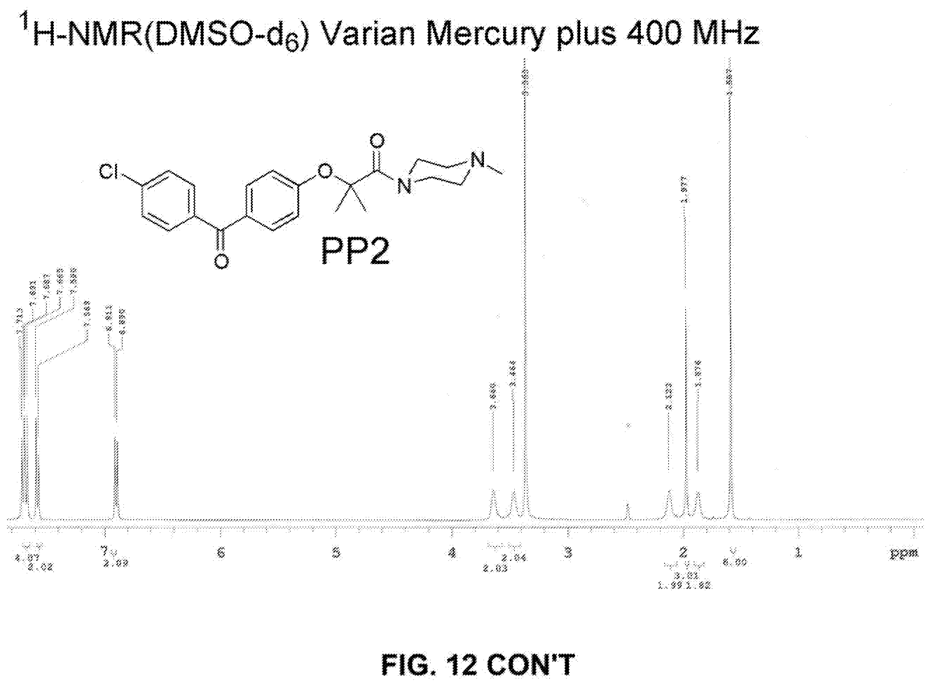

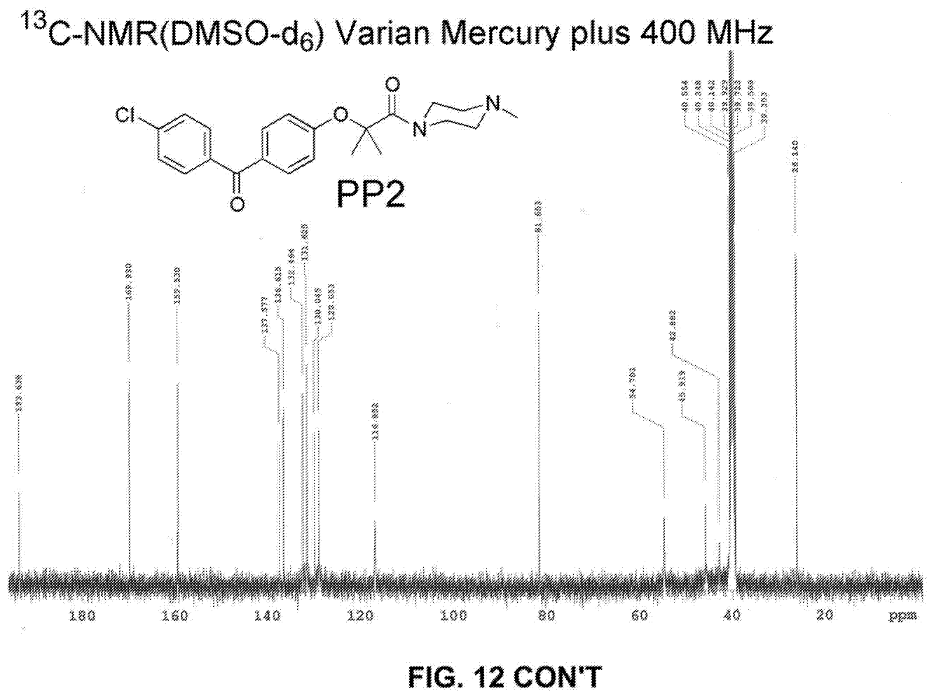

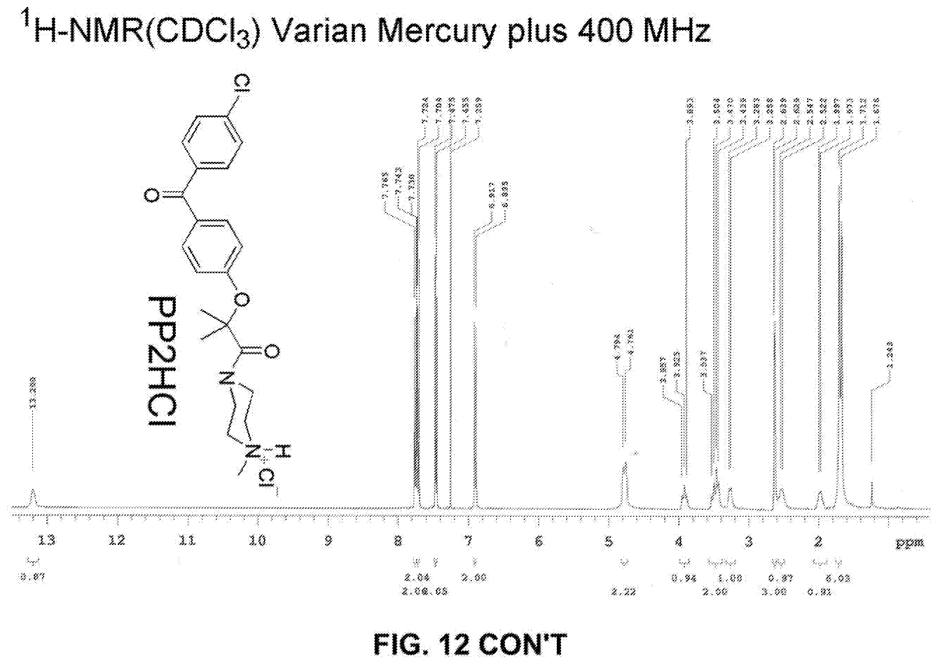

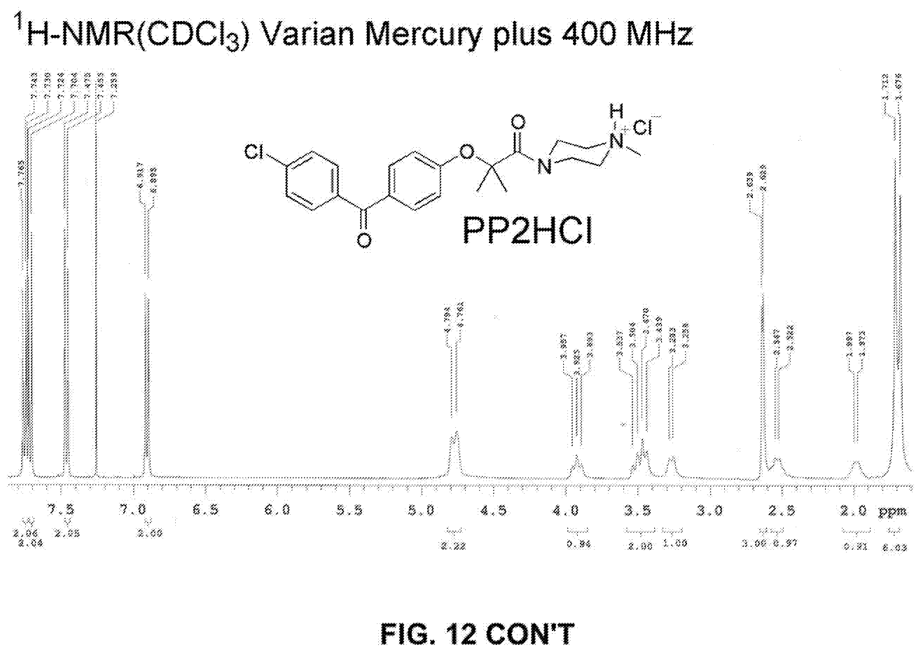

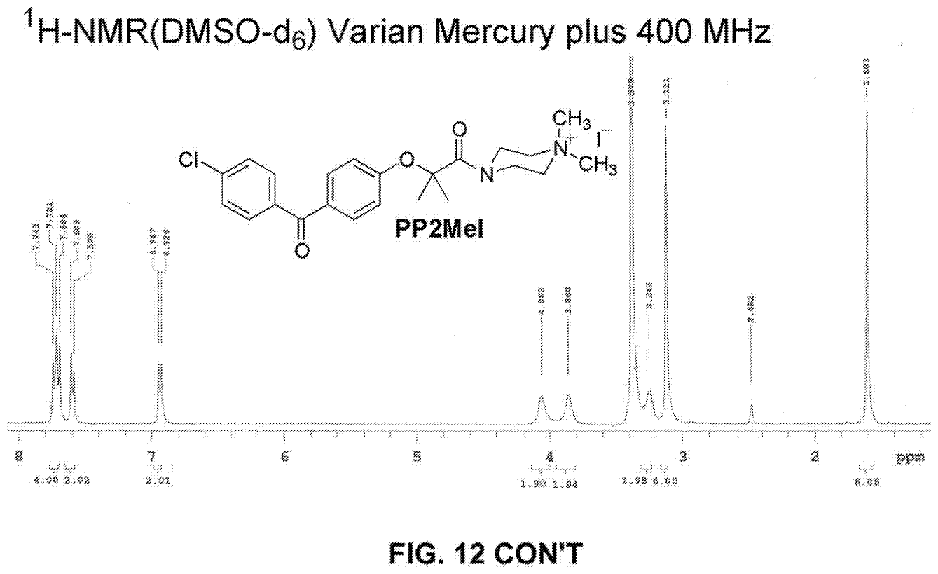

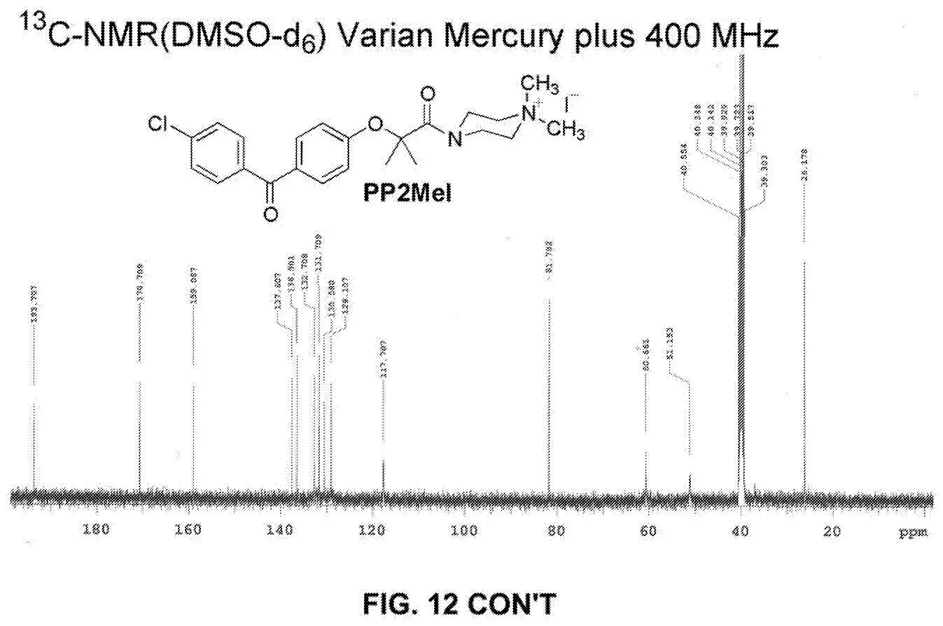

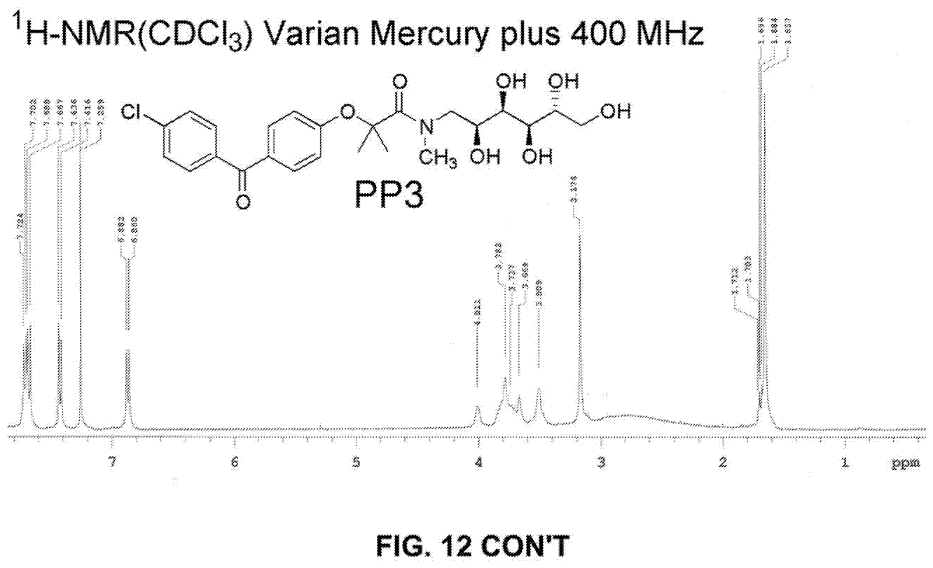

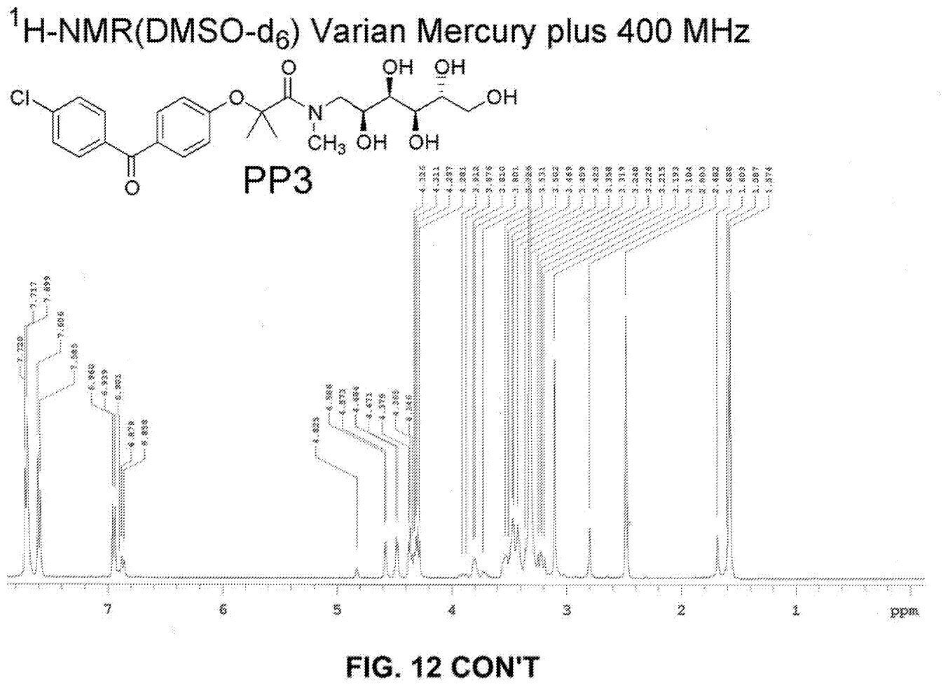

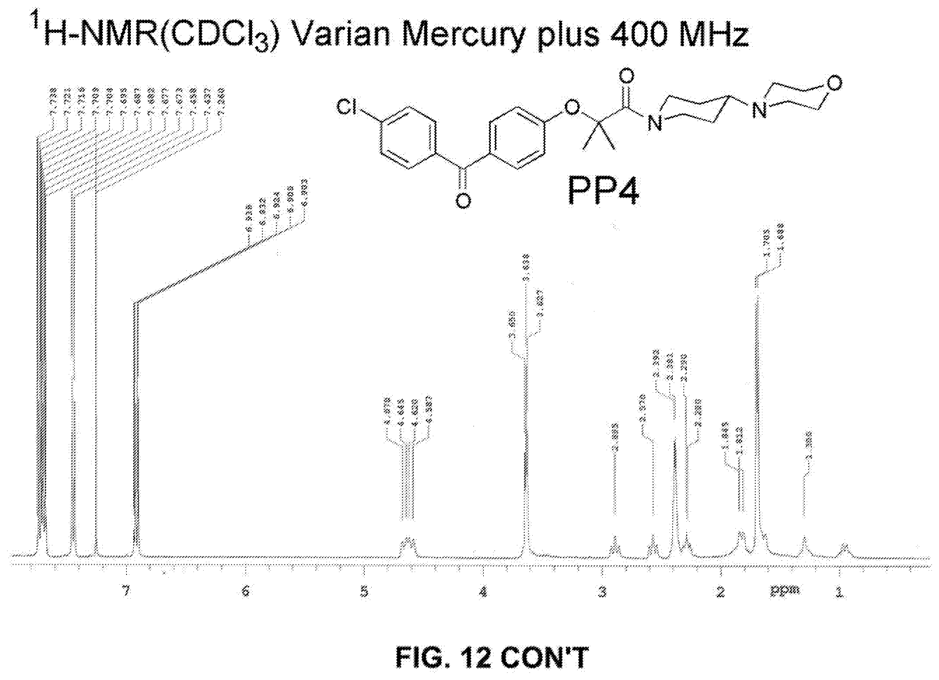

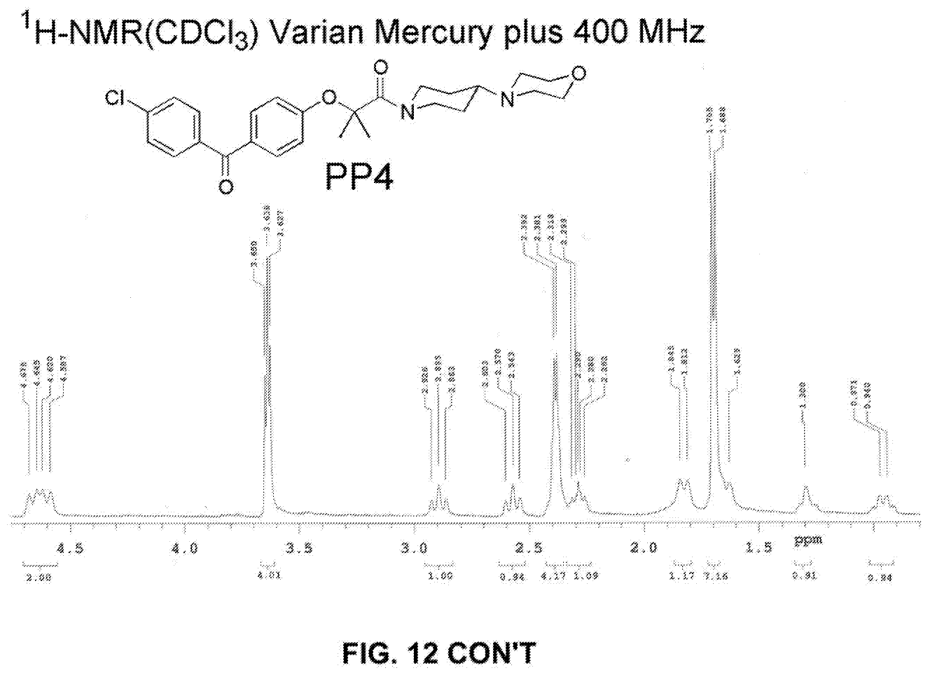

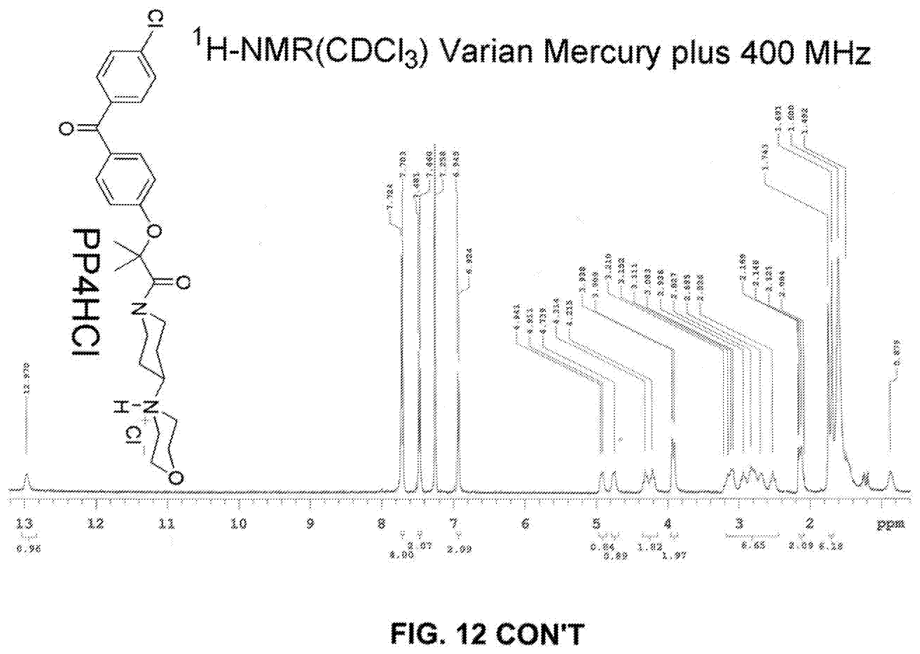

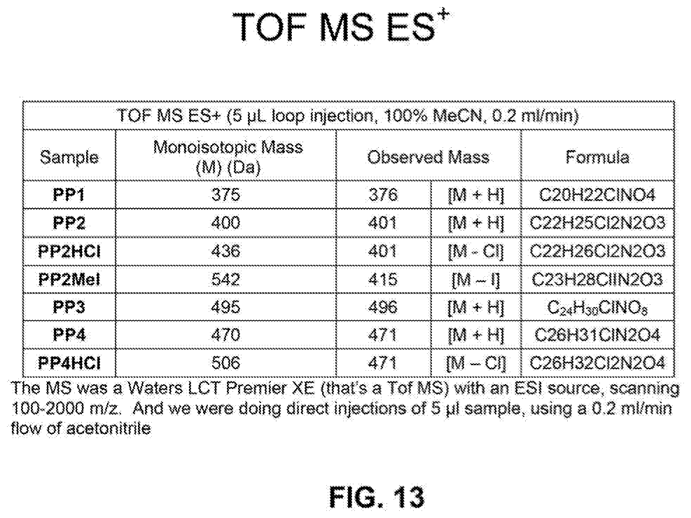

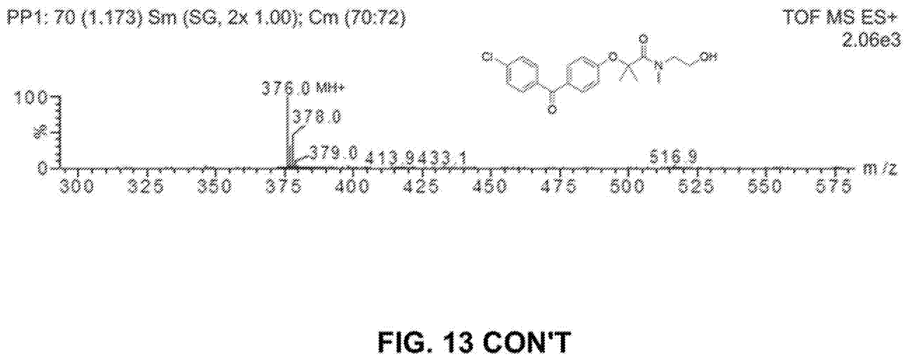

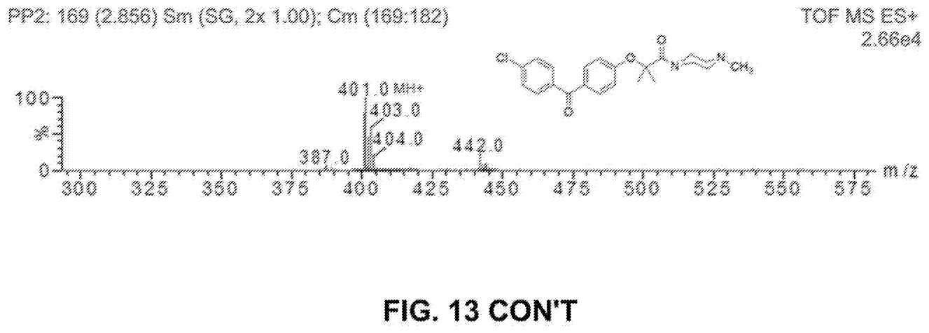

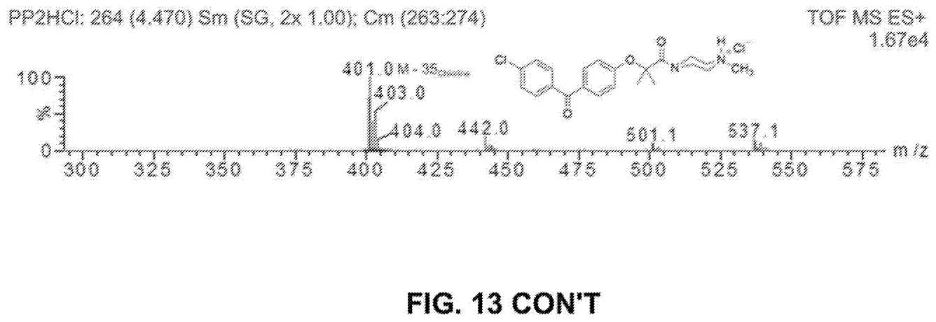

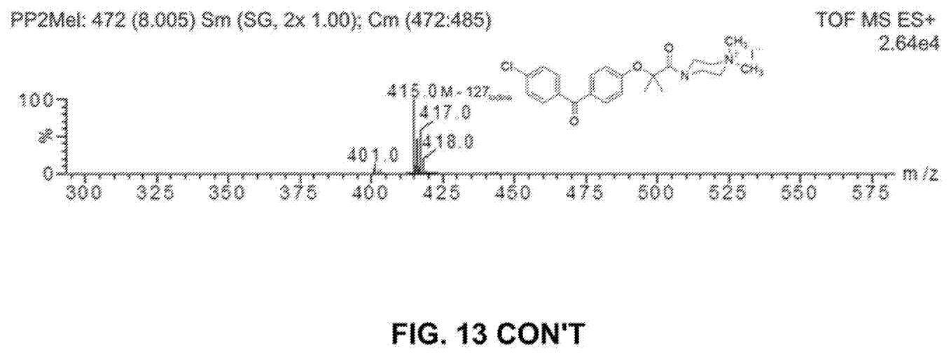

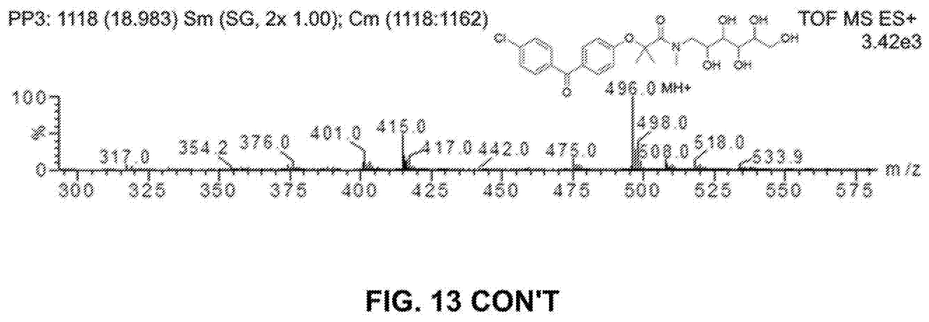

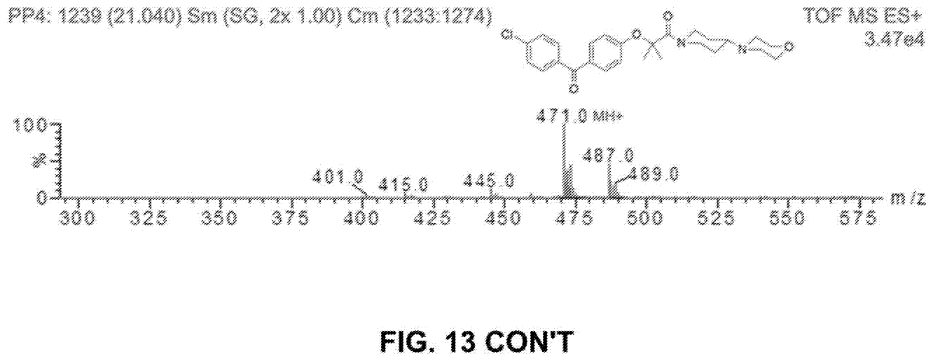

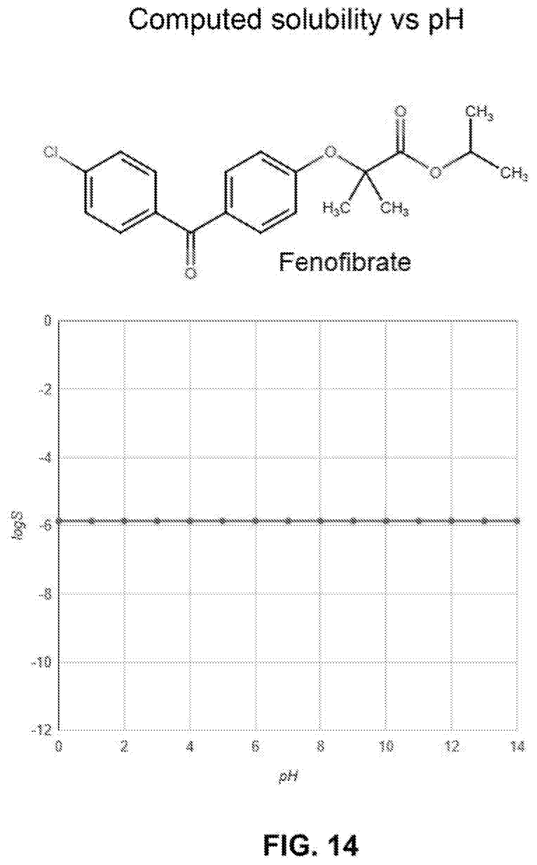

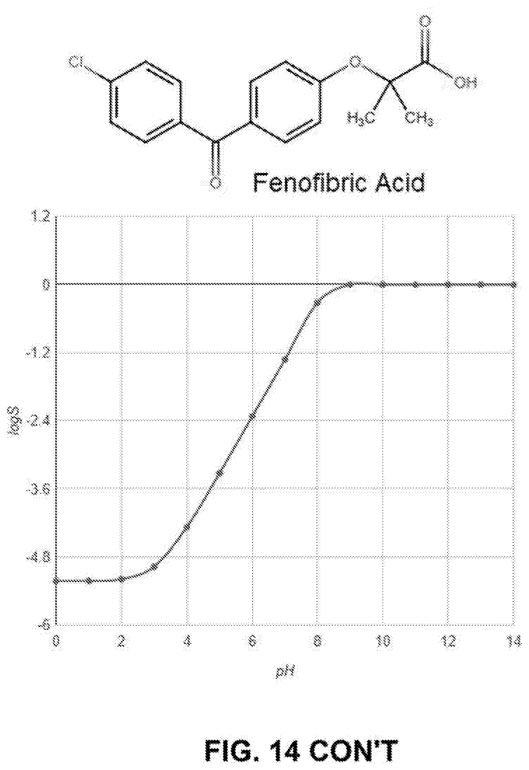

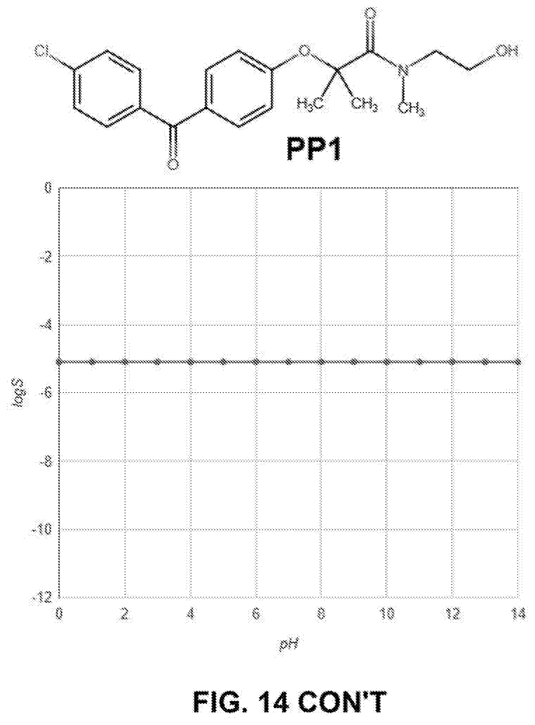

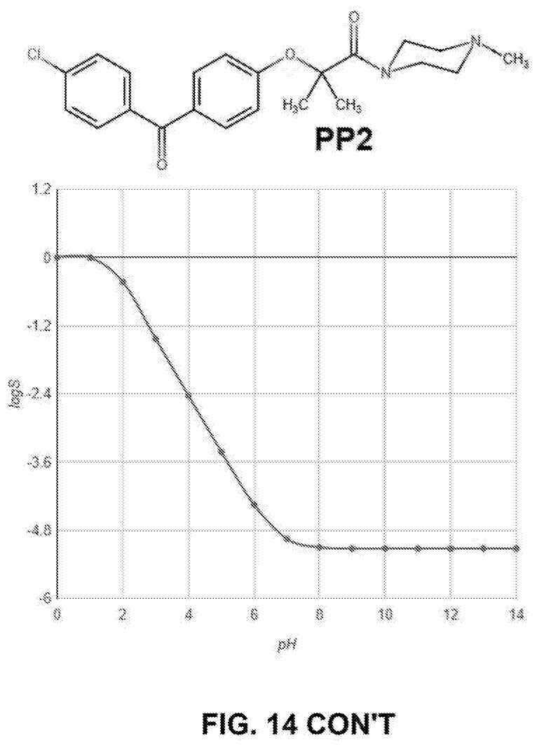

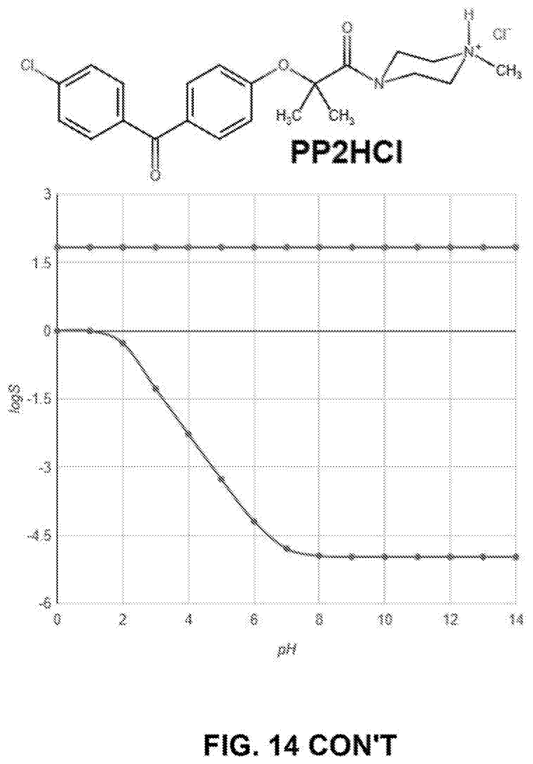

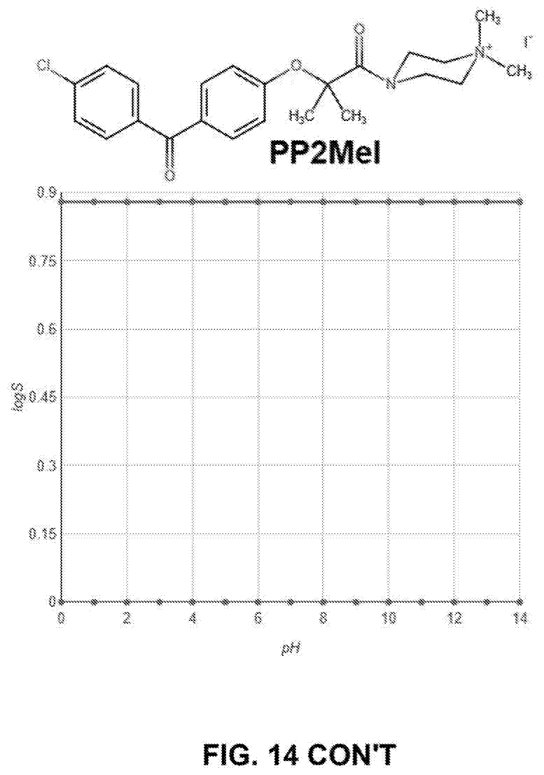

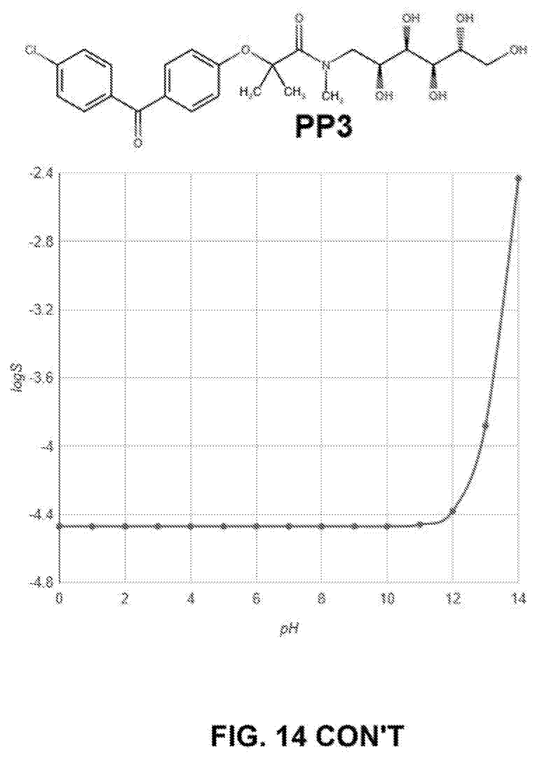

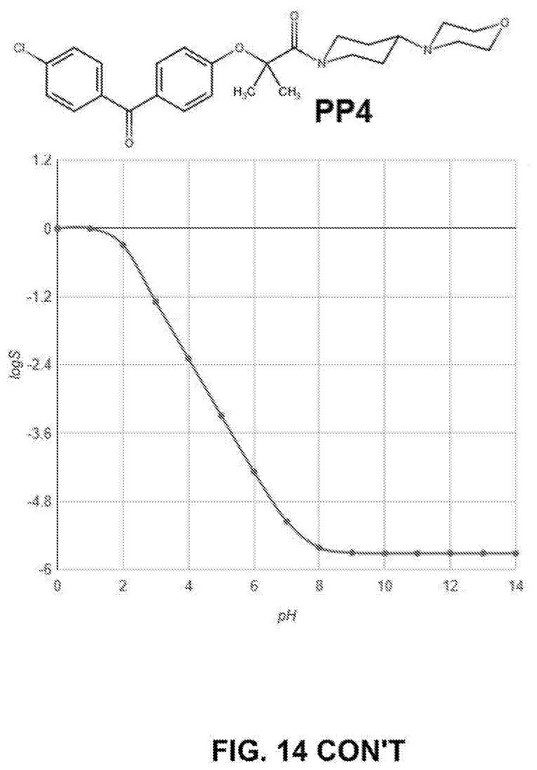

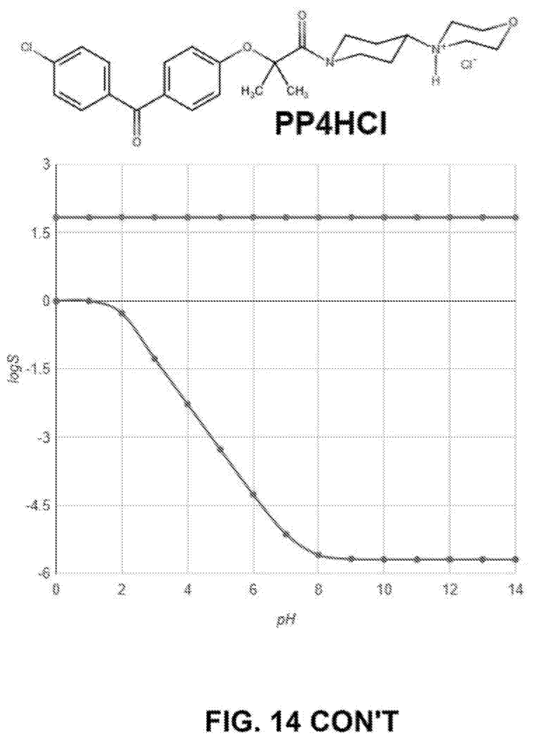

Glioblastoma multiforme (GBM) is a highly lethal brain tumor for which therapeutic options are limited. Rapidly growing and highly invasive GBM cells rely on both glycolysis and mitochondrial respiration to generate sufficient amounts of ATP and intermediate metabolites (anaplerosis). Interfering with these pathways may be a promising therapeutic strategy to induce “metabolic catastrophe” in these practically incurable brain neoplasms. We have demonstrated that tumor cells of neuroectodermal origin, including melanoma, medulloblastoma and glioblastoma, are highly sensitive to the metabolic drug, fenofibrate (FF) (1-11). FF is routinely used as a lipid-lowering drug through the ability of its metabolite, fenofibric acid (FA), to activate peroxisome proliferator activated receptor alpha (PPARa) (12). Although activation of PPARa may explain some of the observed anticancer effects, glioblastoma cells treated with PPARa siRNA retain sensitivity to FF, indicating a PPARa-independent mechanism of its anticancer action (11). Indeed, our previously published data demonstrate that unprocessed FF (ester) accumulates in mitochondrial membranes, with evidence that mitochondrial FF triggers a severe and immediate inhibition of mitochondrial respiration. This leads to a severe decline in intracellular ATP, and apoptotic tumor cell death (11). We also reported, that FF is promptly processed to FA by blood and tissue esterases, and FA is much less effective in triggering tumor cell death, and that both FF and FA do not cross the blood brain tumor barrier (BBTB) (4). To address these issues, which are hampering development of more effective FF-based anti-tumoral therapies, we have made several chemical modifications to improve FF stability, water solubility, tissue penetration, and ultimately, anti-glioblastoma efficacy. Our data show that in comparison to FF, four compounds, designated as PP1, PP2, PP3, and PP4 (FIG. 1), have improved cytotoxicity against glioblastoma cells in vitro (FIG. 2 and FIG. 3); similar to FF, they block mitochondrial respiration (FIG. 5). In addition, PP1 is significantly more stable when exposed to human blood (FIG. 4A), and is much better soluble in water (FIG. 4B). We have also demonstrated that mice treated with PP1—oral administration accumulated PP1 at therapeutically relevant concentrations in several tissues including intracranial glioblastoma tumors (FIG. 6A), and survived the treatment without any major signs of distress (FIGS. 6B and 6D). Importantly, PP1 treatment of mice bearing large intracranial glioblastoma tumors, resulted in extensive areas of necrosis within the tumor mass, thus further supporting anti-glioblastoma efficacy of this new metabolically active compound (FIG. 6D).

Materials and Methods

Chemical procedures to prepare PP compounds—All starting materials were reagent grade purchased from Sigma-Aldrich or Ark Pharm. ¹H-NMR spectra were recorded on Varian Mercury Plus 400 MHz instrument in CDCl3 or DMSO-d6, with the solvent chemical shifts as an internal standard. All computed molecular descriptors were generated by Chemaxon MarvinSketch version 18.8.0.

Preparation of 2-[4-(4-chlorobenzoyl)phenoxy]-N-(2-hydroxyethyl)-N,2-dimethylpropanamide (PP1)—

Method A: Dichloromethane (20 ml) suspension of fenofibric acid (318.75 mg; 1 mmol) and oxalyl chloride (0.25 mL; 380.1 mg; 3 mmol), and one drop od N,N-dimethylformamide was stirred at room temperature for 5 hours. Solvent was evaporated under reduced pressure. White solid residue was resolved in dichloromethane (10 ml) and again evaporated to the solid residue. This solid residue was dissolved in dichloromethane (20 ml) and at room temperature with stirring dichloromethane (10 ml) solution of 2-(methylamino)ethanol (0.24 ml; 225 mg; 3 mmol) was gradually added. Reaction mixture was stirred at room temperature. Dichloromethane reaction mixture was washed with water (3×15 ml), 5% hydrochloric acid (3×15 ml), water (3×15 ml), 10% sodium carbonate (3×15 ml), and finally with water (3×15 ml) and dried over anhydrous sodium carbonate. Solvent was evaporated under reduced pressure to result in viscous pale-yellow liquid (390 mg). Product was crystalized from dichloromethane/hexane (30 ml; 1:4) at room temperature by slow solvent evaporation to ⅕ original volume and formed crystals were washed with ice cold hexane. Isolated yield 340 mg (90%).

Method B: Dichloromethane (100 ml) solution of fenofibric acid (637 mg; 2 mmol), 1-ethyl-3-(3-dimethylaminopropyl)carbodiimide, hydrochloride (EDC, 576; 3 mmol), and 2-(methylamino)ethanol (600 mg; 8 mmol) was stirred at room temperature overnight. Dichloromethane solution was washed with 5% hydrochloric acid (5×20 ml), water (5×20 ml), 10% sodium carbonate (5×20 ml), water (3×20 ml) and dried over anhydrous sodium carbonate. After solvent evaporation, oily residue was crystalized from dichloromethane/hexane (1:4) to give pure product (640 mg; 85% yield). ¹H-NMR (CDCl₃) δ 7.74 (2H, 6, J=8.8 Hz), 7.71 (2H, d, J=8.8 Hz), 7.45 (2H, d, J=8.8 Hz), 6.92 (2H, d, J=8.8 Hz), 3.78 (2H, t, J=4.8 Hz), 3.53 (2H, t, J=4.8 Hz), 3.6 (1H, broad s), 3.17 (3H, s), and 1.71 (6H, s) ppm. ¹³C-NMR (CDCl₃) δ 194.1, 173.5, 159.3, 138.3, 136.1, 132.2, 131.1, 130.3, 128.5, 116.5, 81.4, 60.3, 52.8, 36.7, and 25.7 ppm.

Preparation of 2-[4-(4-chlorobenzoyl)phenoxy]-2-methyl-1-(4-methylpiperazin-1-yl)propan-1-one (PP2)—Dichloromethane (30 ml) of fenofibric chloride (1 mmol; prepared as described above for PP1 preparation) was slowly added in stirring water (5 ml) solution of sodium carbonate (216 mg; 2 mmol) with tetrahydrofuran (10 ml) and of 1-methylpiperazine (0.13 ml; 120 mg; 1.2 mmol). Resulting reaction mixture was stirred at room temperature for one hour. Additional water (30 ml) was added and organic layer was separated, washed with water (3×10 ml), 5% hydrochloric acid (3×10 ml), 10% sodium carbonate (3×10 ml), water and dried over anhydrous sodium carbonate. After evaporation oily residue was crystalized from dichloromethane hexane to give 350 mg (88%) of pure product. 1H-NMR (DMSO-d6) δ 7.71 (2H, d, J=8.8 Hz), 7.67 (2H, d, J=8.8 Hz), 7.58 (2H, d, J=8.4 Hz), 6.90 (2H, d, J=8.4 Hz), 3.64 (2H, broad s), 3.46 (2H, broad s), 2.12 (2H, broad s), 1.98 (3H, s), and 1.59 (6H, s) ppm. 13C-NMR (DMSO-d6) δ 193.6, 169.0, 159.5, 137.6, 136.6, 132.5, 131.6, 130.1, 129.1, 116.8, 81.7, 54.7, 45.9, 42.9 and 26.1 ppm.

Preparation of 2-[4-(4-chlorobenzoyl)phenoxy]-N,2-dimethyl-N-[(2S,3R,4R,5R)-2,3,4,5,6-pentahydroxyhexyl]propenamide (PP3)—Fenofibric acid chloride prepared from fenofibric acid (318.75 mg; 1 mmol) and oxalyl chloride (0.25 mL; 380.1 mg; 3 mmol) as described for preparation of PP1 was dissolved in dichloromethane (15 ml) and mixed with acetonitrile (20 ml) and water (10 ml) solution of N-methyl-D-glucamine (196 mg; 1 mmol) and sodium carbonate (212 mg; 2 mmol). Resulting mixture was stirred at room temperature for five minutes and solvent was evaporated under reduce pressure at room temperature. Resulting solid residue was mixed with dichloromethane (50 ml) and water (20 ml). Dichloromethane layer was separated, washed with 10% sodium carbonate (3×10 ml), water (3×10 ml) and dried over anhydrous sodium carbonate. After solvent was evaporated solid residue was washed with hexane (3×5 ml) and dried in vacuum under reduce pressure to give 350 mg (71%) of pure product. Selected signals for ¹H-NMR (CDCl₃) δ 7.71 (2H, d, J=8.8 Hz), 7.67 (2H, d, J=8.8 Hz), 3.17 (3H, s), and 1.66 (6H, s) ppm.

Preparation of 2-[4-(4-chlorobenzoyl)phenoxy]-2-methyl-1-[4-(morpholin-4-yl)piperidin-1-yl]propan-1-one (PP4)—Fenofibric acid chloride prepared from fenofibric acid (160 mg; 0.5 mmol) and oxalyl chloride (0.25 mL; 380.1 mg; 3 mmol) as described for preparation of PP1 was dissolved in dichloromethane (30 ml) and mixed with tetrahydrofuran (10 ml) solution of 4-morpholinopiperidine (100 mg; 0.6 mol), and water (10 ml) solution of sodium carbonate (106 mg; 1 mmol). Resulting mixture was stirred at room temperature for one hour. Water (20 ml) was added and organic layer was separated and extensively washed with 5% hydrochloric acid (5×20 ml), water (3×10 ml), 10% sodium carbonate (5×20 ml) and again with water (3×10 ml). After drying over anhydrous sodium carbonate solvent was evaporated to give pure product (200 mg; 85%) as oil that crystallized by standing at room temperature overnight. If necessary, the product can be further purified by crystallization from hexane or by silica gel chromatography with ethyl acetate-ethanol (5:1). ¹H-NMR (CDCl₃) δ 7.71 (2H, d, J=7.6 Hz), 7.68 (2H, d, J=7.6 Hz), 7.47 (2H, d, J=7.6 Hz), 6.92 (2H, d, J=7.6 Hz), 4.66 (1H, d, J=13.2 Hz), 4.60 (1H, d, J=13.2 Hz), 3.64 (4H, t, J=4.8 Hz), 2.90 (1H, t, J=12.8 Hz), 2.57 (1H, t, J=13.2 Hz), 4.38 (4H, m), 2.28 (1H, t of t, J1=11.2 Hz, J2=4 Hz), 1.83 (1H, d, J=13.2 Hz), 1.71 (6H, s), 1.64 (1H, d, J=13.2 Hz), 1.30 (1H, m), and 0.95 (1H, m) ppm.

Preparation of 4-(1-{2-[4-(4-chlorobenzoyl)phenoxy]-2-methylpropanoyl}piperidin-4-yl)morpholin-4-iumchloride (PP4HCl)—A mixture of concentrated hydrochloric acid (2 ml) and PP4 (94 mg; 0.2 mmol) was sonicated at room temperature for half an hour. Clear solution was evaporated to dryness at room temperature under reduced pressure. Residue was dissolved in dry ether (20 ml) and clear solution was left at room temperature for solvent to slowly evaporate. For white crystalline material was separated by filtration and washed with ice-cold hexane to give 80 mg (79%) of pure product. 1H-NMR (CDCl₃) δ 12.97 (1H, s), 7.71 (4H, d, J=8.4 Hz), 7.47 (2H, d, J=8.4 Hz), 6.93 (2H, d, J=8.4 Hz), 4.93 (1H, d, J=12.0 Hz), 4.74 (1H, d, J=12.0 Hz), 4.31 (1H, t), 4.22 (1H, t), 3.92 (2H, d, J=11.6 Hz), 2.4-3.2 (7H, m), 2.13 (2H, m), 1.60 (6H, s), 1.10 (1H, m), and 0.88 (1H, m) ppm.

Detection of PP compounds by high performance liquid chromatography (HPLC) —All HPLC data were obtained from the Agilent 1100 apparatus equipped with a line degasser, binary pump (high pressure mixer), autosampler, column thermostat and Diode Array Detector (DAD) (Agilent Technologies, Santa Clara, Calif.). The analytical column 3 μm, 4.6×150 mm (Octyl Silane C8; YMC America, Inc.), solvent A—50 mM acetic acid in water, and solvent B—acetonitrile, with isocratic flow were used to detect and quantify PP compounds in culture media, in cells, tissues and body fluids. The flow rate was set to 1 ml/min, column temperature was 20° C., and the sample volume was 5 μl. DAD wavelength was set to 285 nm. Sample preparation. Blood, cell culture media, cellular and tissue lysates, were deproteinized by adding 150 μl of acetonitrile to 150 μl of sample, mixed well and centrifuged (15 000 g, 5 min). The lysates were sonicated on ice and centrifuged (15 000 g, 5 min). Finally, 150 μl of the supernatant was mixed with the equal volume of acetonitrile, filtered through 0.22 μm centrifuge filter (Sigma) and analyzed by High Performance Liquid Chromatography (HPLC).

Cell Culture—We have used two human glioblastoma cell lines LN-229 (ATCC #CRL-2611) and U-87MG (ATCC #HTB14); GBM12, which are patient-derived human glioblastoma cells (13, 14); and mouse glioblastoma cell line Bioware Brite GL261-Red-FLuc (PerkinElmer #BW134246). All cell lines were maintained as semi-confluent monolayer cultures in DMEM (1 g/L glucose; with sodium pyruvate and L-glutamine) supplemented with 100 U/ml penicillin, 100 μg/ml streptomycin, and 10% fetal bovine serum (FBS) at 37° C. in a 5% CO2 atmosphere. The cells were treated with the PP compounds at different doses ranging from 5 to 50 μM. In addition, the cells were treated with fenofibrate (FF; Sigma Aldrich, St. Louis), at concentrations ranging from 10 to 50 μM. Control cultures were treated with the corresponding volumes of DMSO (vehicle control; final concentration 0.1%). GBM12 cells were routinely propagated in the subcutaneous tissue of nude mice and isolated from the tumor tissue for short-term cultures as previously described (11), and according to IACUC protocol #3444 5 (LSUHSC, New Orleans).

Evaluation of metabolic parameters—Metabolic responses of human glioblastoma cells were evaluated with Extracellular Flux Analyzer XFe24 (Agilent Technologies). Day prior to each assay the cells were plated at 4×104 cells/well in Agilent Seahorse 24-well XF cell culture microplates in growth supporting media and incubated overnight. At the time of measurement, growth media were replaced with serum-free XF assay medium (Seahorse XF Base Medium supplemented with 1 mM sodium pyruvate, 2 mM glutamine, and 5.5 mM glucose) and cartridges equipped with oxygen-sensitive and pH-sensitive fluorescent probes (Seahorse) were placed above the cells. The oxygen consumption rate (OCR; indicative of mitochondrial respiration) and acidification rate (ECAR; indicative of glycolysis) were evaluated after injecting the PP compounds (all used at 25 μM), FF (50 μM) or DMSO (0.1%; vehicle control) followed by injections of metabolic toxins: oligomycin (inhibitor of ATP synthase; complex V of the Electron Transport Chain, ETC; 0.5 μM), carbonylcyanide-p-trifluoromethoxyphenylhydrazone (FCCP; uncoupling factor; 0.5 μM), rotenone (inhibitor of mitochondrial complex I of ETC; 0.3 μM), and antimycin A (inhibitor of mitochondrial complex III of ETC; 0.3 μM).

PPAR Luciferase Assay—The PPAR transcriptional activity was determined by utilizing the JsTkpGL3 reporter plasmid, which contains a firefly luciferase gene driven by the PPAR responsive element (PPRE), which consists of three copies of the J site from the apo-AII gene promoter. Together with JsTkpGL3 plasmid HepG2 cells were transfected with pSV40-GLuc (New England Biolabs, Ipswich, Mass.) control plasmid expressing Gaussia luciferase under the control of the constitutive SV40 early promoter to normalize for efficiency of transfection. Twenty-four hours after transfection the cells were incubated with ciglitazone (30 μM), fenofibrate, PP1, PP2, PP3 and PP4 (all 25 μM) for an additional 24 hrs. The luciferase activity was detected with Dual-Luciferase reporter assay system (Promega, Madison, Wis.), and the resulting luminescence measured with Synergy 2 microplate reader (BioTek, Winooski, Vt.).

Cell death assays—Cell death was evaluated by assays based on cell membrane integrity. We used either the trypan blue exclusion test (15) or GUAVA easyCyte 8HT flow cytometer with ViaCount reagent (Millipore) and Guava/ViaCount software for data analysis. Briefly, the cells were plated at 1×104 cells/cm2 in 24-well plates in growth medium. After 24 hours, the medium was replaced by fresh growth medium containing PP compounds, FF or 0.1% DMSO (vehicle control) and further incubated for the amount of time specified for each experiment. The cells were then harvested with 0.05% trypsin/EDTA, centrifuged, re-suspended in PBS and counted in a hemocytometer with trypan blue (0.4%, 1:1) or incubated with the ViaCount reagent (1:10; 5 minutes at room temperature) before cell viability was assessed by Guava/ViaCount according to the manufacturer's recommendations.

Animal studies—All described procedures involving experimental animals were performed in accordance to the IACUC protocol #3444 at LSUHSC, New Orleans. C57BL/6NHsd mice, 11-12 weeks of age (Envigo), were anesthetized with 4% isoflurane and secured in a stereotaxic head frame (Harvard Apparatus, Holliston Mass.). GL-261-luc cells (1×105) (PerkinElmer Inc.) were suspended in PBS and 2 μl aliquots were injected into the brain parenchyma (coordinates: 3 mm anterior to Bregma; 1.5 mm lateral to Sagital suture; 3 mm down from surface) through a burr hole in the skull using a 10 μl Hamilton syringe. Biophotonic images of the skull were captured using a Xenogen IVIS 200 imaging system (Palo Alto, Calif.) two weeks after initial cell implantation (FIG. 6C). Prior to imaging, each mouse received an intraperitoneal injection of 100 μl of D-luciferin (30 mg/ml solution; PerkinElmer, Waltham, Mass.), and anesthetized by isoflurane inhalation. The resulting images were evaluated and luminescence measurements from equivalent regions of interest encompassing the entire skull were collected using Living Image 4.1 software (Xenogen). Treatment: Control C57BL mice and C57BL bearing well established intracranial mouse glioblastoma tumors (GL-261) were treated with the PP1 (50 mg/kg/day) administered by the oral gavage. Following 14 days of daily drug administration the animals were euthanized according to the standard ethically accepted procedure, and the following organs/body fluids were collected: blood, liver, kidneys, spleen, heart, intact brain and intracranial tumors. These tissues were subjected to sample preparation for the HPLC analysis (PP1 tissue content), and for the routine pathological evaluation.

Pathological evaluations—After harvesting the brain and tumors, liver, spleen, kidneys, heart and lungs, the tissues were placed in 10% buffered formalin for 24 hours, processed and embedded into paraffin blocks. Sections were cut at 4 microns in thickness, placed in electromagnetically charged slides, deparaffinized, rehydrated and stained with Hematoxylin and Eosin for routine histopathological examination.

Statistical analysis—The data were analyzed with Student's t-test corrected for multiple comparisons using Bonferroni-Dunn method. The difference between control and experimental groups were considered significant and marked with an asterisk (*) for P values lower or equal 0.05.

Results

Synthesis of new FF-based compounds for glioblastoma therapy—We have modified certain specific physiochemical properties of FF to improve its resistance to blood and tissue esterases, water solubility, tissue uptake and ultimately anticancer activity. As a result, we have initially generated 23 new compounds, which have been designated here as PP compounds. They all contain a structural motif outlined in FIG. 1A. For example, we have replaced the ester group that is present in FF with different amide groups. This is because, amides hydrolysis rate under physiological conditions is substantially slower than esters (16, 17). In contrast, esters such as FF are readily cleaved by hydrolytic reactions catalyzed by acids, bases, metal ions, and hydrolytic proteins such as human serum albumin (18), and more specifically by blood and tissue esterases (4, 19-22). Unless designed with specific functional groups, amide bonds are rarely cleaved by chemical hydrolysis under physiological conditions, and their chemical cleavage requires harsh conditions such as high temperature in combination with the presence of strong acids or bases (16). In addition to higher resistance to the hydrolyses, tertiary amides have higher waters solubility as compared to esters, and to primary, or secondary amides. Based on our computational and biological studies, we have initially selected four compounds (PP1-PP4) for further analyses (FIG. 1A). Preparation procedures for these compounds is outlined in FIG. 1B and described herein, such as in the Methods section. There are two feasible methods that are commonly used in organic synthesis for preparation of amides from carboxylic acid: (a) through acid chlorides and (b) by activating carboxylic acid with carbodiimides (23). Fenofibric acid (FA) was used as a primary substrate, and was converted in to the corresponding acid chloride with oxalyl chloride. This chloride was coupled with secondary amine in basic media resulting in preparation of the PP compounds. Isolated yields were good to excellent and the products (PP1-PP4) were purified by extraction and crystallization.

Computed physicochemical properties of the four preselected PP compounds are presented in Table 1. Using computational methods for the estimation of physicochemical properties of potential new lead compounds is well established in medicinal chemistry (24, 25). If we compare our computed descriptors to one obtained from lead compounds with anticancer activity (25), our amides PP1, PP2, and PP4 are well in the desired range (Table 1). Molecular weight should be around 380, C log P around 3.7, PSA around 80, and HBA around 5. On the other hand, PP3 was designed to substantially increase water solubility and it is a saccharide derivative that was perfectly reflected on its estimated physicochemical properties. It is well hydrated (5 hydrogen bond donors and 16 hydrogen bond acceptors) in water media, it is hydrophilic (low C log P), however, it has a large polar surface area that might decrease its cell membrane permeability (26).

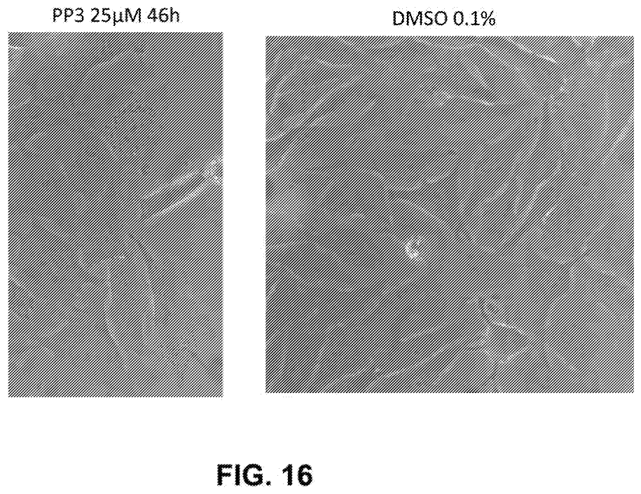

In vitro anti-cancer effects of PP compounds compared to fenofibrate (FF)—Since fenofibric acid (FA), was used as a primary substrate for the proposed chemical modifications (FIG. 1), and FA has only marginal anticancer properties in comparison to unprocessed FF (11), we have tested first if the new compounds (PP1-PP4) are indeed cytotoxic. We used two human glioblastoma cell lines (LN-229 and U87MG); patient derived glioblastoma cells (GBM12); and mouse glioblastoma cell line (GL-261) in this evaluation. Results in FIG. 2A demonstrate changes in the percentage of cell death in LN-229 cells cultured in 10% FBS+/−PP1, PP2, PP3 and PP4. All compounds were used at 5, 10, 25 and 50 μM, and the cells were treated for 24, 48, 72 and 96 hrs. The control cultures were treated either with an equal volume of the vehicle (DMSO), or with 25 and 50 μM FF (positive control). In DMSO treated cultures, the average cell death varied from 6+/−1.4% to 7+/−1.2% (FIGS. 2A and 2B). In the presence of 50 μM FF (single dose), LN-229 cells demonstrated 24+/−4% cell death at 48 hours; 90+/−1% at 72 hours, and 99+/−1.2% at 96 hrs. In contrast, 25 μM FF was not cytotoxic; however, the treated cells demonstrated a significant growth arrest (FIG. 2B). Next, we compared FF data to PP1, PP2, PP3 and PP4. Similar to FF, all four compounds did not show any cytotoxic effects during first 48 hours following the treatment at all concentrations (FIG. 2A). At 72- and 96-hour time points, PP1, PP2 and PP4 triggered extensive cell death at both 25 and 50 μM. This strong cytotoxicity was also observed at 10 μM PP1 (92+/−2% cell death), and at 5 μM PP1 (74+/−3% cell death); and all four compounds were cytostatic at 5 μM (FIG. 2C-2F), and FF was cytostatic at 25 μM (FIG. 2B). In difference to PP1, PP2 and PP4; PP3 demonstrated kinetics of growth retardation and cytotoxicity similar to FF (FIGS. 2A and 2B). Like FF, PP3 was cytotoxic only at 50 μM, and demonstrated cytostatic activity at 10 μM (FIGS. 2A and 2B). We also observed extensive accumulation of peroxisomes in cells treated with PP3, which resembled morphology of LN-229 cells treated with 50 μM FF (FIG. 16). This is also consistent with the data showing that PP3 does not repress PPAR responsive elements (PPRE) (FIG. 4C), therefore, its anticancer activity could be different from PP1, PP2 and PP4, and more similar to FF. However, PP3 dissolved in DMSO is quite unstable, and needs to be prepared fresh for each experiment. For example, an old stock solution of PP3 may be less active in inducing tumor cell death than a fresh stock.

On the bases of these initial findings, we have selected PP1 for additional experiments and confirmed its high in vitro cytotoxicity in another human glioblastoma cell line, U87MG (FIG. 3A), in patient derived glioblastoma cells GBM12 (13, 27) (FIG. 3B), and in mouse glioblastoma cell line, GL261 (FIG. 3C).

Physiochemical and Metabolic effects of PP compounds compared to FF—The main purpose for the described chemical modifications was to generate new compounds, which in comparison to FF, are more stable, resistant to blood and tissue esterases, better soluble in water, and possibly more effective in penetrating blood brain tumor barrier (BBTB). Our data show that in comparison to FF, PP1 was significantly more resistant to blood esterases (FIG. 4A). In this experiment, 50 μM of PP1 and 50 μM of FF were incubated with human blood at 370 C, at indicated time points. Following 48-hour incubation, almost all FF was converted to FA (FIG. 4A; right panel). In contrast, nearly 25 μM of PP1 was still detected at the 48-hour time point (FIG. 4A; left panel). In addition, we also demonstrate that in comparison to FF, PP1 is much better soluble in water (FIG. 4B), further supporting its potential as a new anticancer drug.

We have also demonstrated that PP1, PP2 and PP4 attenuate PPAR responsive elements (PPRE) (FIG. 4C). This unexpected finding indicates that these three new compounds act differently in comparison to FF, which following its conversion to FA, is a potent agonist of PPARα. In contrast, PP3 does not share this PPAR-inhibitory activity, and its action on glioblastoma tumor cells could be more similar to FF than to other PP compounds (see also FIGS. 2A and 2B).

Since FF anticancer effects are mediated mainly by the inhibition of mitochondrial respiration (11), we used an Extracellular Flux Analyzer (XF24, Seahorse Biosciences) to measure real-time oxygen consumption rate (OCR; indicative of mitochondrial respiration) and extracellular acidification rate (ECAR; indicative of glycolytic activity) in LN229 human glioblastoma cells treated with the PP1 at 25 μM concentration. The cells treated with DMSO (vehicle) or with 25 μM FF were used as a background control and positive control, respectively. These metabolic parameters were measured in monolayer cultures after sequential injections of the following metabolic toxins: oligomycin [inhibitor of complex V (ATP synthase)], FCCP (mitochondrial uncoupling factor); rotenone [inhibitor of mitochondrial complex I (NADH dehydrogenase)], and antimycin A (inhibitor of mitochondrial complex III). In a typical “mitochondrial stress” experiment (FIG. 5), addition of FF and PP1 resulted in a dramatic decrease of OCR values and the corresponding increases in ECAR. Similar results were obtained for PP2, PP3 and PP4 (not shown). OCR and ECAR values in the control (DMSO) were practically unaffected (blue plot). In DMSO-treated controls, addition of oilgomycin inhibited OCR, addition of FCCP resulted in a maximal increase of OCR values, and finally addition of rotenone completely blocked the oxygen consumption, as expected (FIGS. 5A and 5B). Conversely, cells treated with either FF or PP1 demonstrated repressed oxygen consumption, accompanied by increased ECAR values (glycolysis) throughout the experiment. Other relevant metabolic parameters calculated from this experiment confirmed extremely low ATP production and almost none existing Spare Respiratory Capacity in the cells treated with either with FF or with PP-compounds (FIG. 5A).

PP1 tissue distribution and toxicity in intracranial glioblastoma—We used a syngeneic mouse glioblastoma model in which GL-261-luc cells (PerkinElmer Inc.) were injected into the brain parenchyma of C57BL/6 mice. Following 2 weeks of a continuous tumor growth, mice with large intracranial tumors were selected by using biophotonic imaging (Xenogen IVIS 200) (FIG. 6C). Control mice were treated with DMSO (vehicle) and experimental mice were treated with the PP1 at 25-75 mg/kg/day, administered by the oral gavage. Following 14 days of daily drug administration the animals were euthanized and the following organs were collected: blood, liver, kidneys, spleen, heart, intact brain and intracranial tumors. Both tumor-free and tumor-bearing mice were used for PP1 tissue distribution (FIG. 6A), and toxicity analyses (FIGS. 6B, 6D and 6E). Results in FIG. 6A demonstrate that PP1 accumulates in all tissues examined, and importantly, an average concentration of the compound found in intracranial tumors was 5.8+/−0.7 μM, could be therapeutically relevant, according to our in vitro data (FIG. 2). The highest PP1 accumulation was found in the liver (10.3+/−4.3 μM) and in the kidney (8.8+/−4.7). In the blood, spleen, heart and intact brains, the levels were 3.05+/−0.2; 7.6+/−3.2; 6.1+/−2.4; and 4.9+/−3.4 μM, respectively. Importantly, both tumor-free and tumor bearing mice treated with PP1 did not show any signs of overall toxicity and maintained their body weight during the course of the treatment (FIG. 6B). However, histological evaluation of the PP1-treated mice demonstrated inflammation and focal necrotic areas in the liver, and enlarged spleen with accumulation of hemosiderin (FIG. 6D). In contrast, heart, kidney and intact brain did not show any signs of pathology (FIG. 6D).

Results in FIG. 6E demonstrate data from mice with the preexisting, well-developed glioblastoma tumors (FIG. 6C). In this experiment, the tumors were allowed to grow for additional 14 days, and mice were treated either with DMSO (vehicle) or with PP1 (50 mg/kg/day—oral administration). At the end of the experiment, both PP1 and DMSO-treated tumors were very large, covering almost half of the hemisphere. Interestingly, PP1-treated mice demonstrated multiple large areas of necrotic tissue within the tumor mass (n=3). In contrast, DMSO-treated mice had only small and sparse areas of necrosis (n=2). All mice were euthanized at the same time, because of the size of intracranial tumors. However, presence of massive necrotic areas found exclusively in PP1-treated animals indicates a promising therapeutic potential of this new anticancer drug.

Discussion

Glial tumors account for nearly 50% of all adult primary intracranial neoplasms, among which GBM is the most aggressive and practically incurable (28, 29). A large variety of different genetic and epigenetic modifications have been found in GBMs, among which p53 mutations, EGF receptor amplification, and PTEN mutations are most common (30). However, gene therapy, molecular and immunological approaches targeting these molecules and their pathways, as well as recently tested antibodies against immune checkpoint inhibitors (31) have yet to produce improvements in patient outcomes. In addition to the introduction of personalized medicine approach to target these specific pathways in glioblastoma patients (32-34), metabolic methods including calorie restriction and ketogenic diet, are surprisingly effective as supplemental therapies for glioblastoma patients (35-37). In addition, interesting anticancer effects of lipid lowering drugs, fibrates and statins have been also reported (3, 8, 38-42), among which a ten-year all-cause mortality study involving 7,722 patients treated with different fibrates revealed that the use of these metabolic compounds was associated with a significantly lower total mortality and reduced probability of death from cancer (43). In cell culture and in animal studies, various members of the fibrate family demonstrated a broad range of anticancer activities (1-3, 10, 41, 42, 44-47). These multiple reports encouraged recent clinical trials in which chronic administration of low doses of FF was tested along with chemotherapeutic agents, minimizing their toxicity and acute side effects in patients with recurrent brain tumors and leukemia (48, 49). Although these beneficial anticancer effects of FF are still suspected to rely on PPAR-dependent mechanism/s of action, we have recently demonstrated that brain tumor cells retain sensitivity to FF in the presence of PPARα antagonists or PPARα siRNA (1, 11), which strongly suggests PPARa-independent mechanism. Other labs also demonstrated that FF could have PPAR-independent cellular effects including: PPAR-independent activation of GDF15 (50); effects of FF on cell membrane fluidity (51); and the FF-induced inhibition of mitochondrial respiration in isolated cardiac and liver mitochondria (52, 53). Therefore, a growing line of evidence supports the interaction of unprocessed FF (ester) with biological membranes, which could be a reason for the observed strong anticancer activity of this lipid-lowering drug. In this respect, we have demonstrated accumulation of FF in the mitochondrial membrane fraction of different glioblastoma cells (11). As a consequence, the affected cells underwent immediate impairment of mitochondrial respiration, followed by a compensatory attempt of increasing glycolysis, which prolonged cell survival for nearly 48 hours. However, prolonged exposure to FF depleted intracellular ATP, and activated AMPK-mTOR-dependent autophagy pathway. Although autophagy gave the affected tumor cells additional 24 hours of survival, it was followed by a massive tumor cell death between 72 and 96 hours of the treatment (11). We have also demonstrated that this metabolic approach (single intratumoral injection of FF) was effective in suppressing growth of small intracranial glioblastoma tumors in mice (11).

In spite of these promising results, FF and FA do not cross blood brain tumor barrier (BBB) (4), and FF is quickly converted to FA in blood and tissues (4) and FIG. 4A). To circumvent these problems, we have made several chemical modifications using FA as a substrate. From the total of 26 new compounds initially synthesized, we have selected four, PP1-PP4 with the most promising anticancer properties (FIG. 1 and Table I). These chemical modifications were applied to improve the compounds stability, water solubility, tissue penetration, and ultimately anticancer potential. One of the lead compounds, PP1, is highly effective in triggered extensive glioblastoma cell death in vitro (FIGS. 2 and 3), and similar to FF, induces immediate repression of mitochondrial respiration (FIG. 5). In deference to FF, PP1 is much more stable when exposed to human blood (FIG. 4A), and is significantly better soluble in water (FIG. 4B). Importantly, our animal data show that PP1 accumulated in the intracranial glioblastoma tumors following oral administration (FIG. 6A), and caused extensive necrotic damage in the established large intracranial glioblastoma tumors (FIG. 6E). In these experiments, PP1-treated mice did not show any signs of distress or tissue toxicity (FIGS. 6B and 6D), which supports the idea of using this new metabolic compound as a part of anti-glioblastoma therapy in the future.

The fact that orally administered PP1 can be found in both intact mouse brain and in brain tumor tissues is extremely important. Brain tumors are particularly difficult to treat due to distinct anatomical and physiological traits of neural tissue and vasculature. The blood-brain tumor barrier (BBTB), although more permeable than blood-brain barrier (BBB), still represents the major obstacles preventing chemotherapeutic agents from reaching therapeutically relevant concentrations. The only FDA-approved chemotherapy drug against GBM, which can cross BBTB is temozolomide (TMZ), however glioblastoma cells quickly develop TMZ-resistance and recurrent tumors are practically incurable (13).

Several strategies have been proposed to circumvent BBB and BBTB, however, their efficacy for glioblastoma treatment are still very low. For instance, carotid artery infusion with hyperosmotic mannitol was shown to temporarily open BBB by inducing shrinkage of endothelial cells and disrupting tight junctions. However, the opening lasts about 30 minutes, leaving a very narrow window for potential drug deliver (54, 55). Another strategy involves the use of vasomodulators including bradykinin or nitric oxide donors, which are also able to transiently increase capillary permeability (56, 57). However, the main caveat associated with the use of bradykinin is its ability to promote glioblastoma cell migration, invasiveness, and tumor angiogenesis (58, 59). Intracranial drug deliveries have been also tested, including intratumoral and intreventricular drug injections, and post-surgical implantation of biodegradable wafers (Glaidel). The major limitation of these intracranial interventions is a slow and diffusion of the drug which is not effective in large tumors (60, 61).

The ability of PP1 to cross BBTB is a highly desirable feature, however, it raises another relevant question: why PP1 triggers extensive cell death in the intracranial tumor tissues (FIG. 6E) and is not toxic to tumor-free normal brain (FIG. 6D). Although we do not have a definitive answer at this time, our previous data show that 50 QM FF is significantly less toxic to normal human astrocytes (NHA) compared to glioblastoma cell lines (11). This is in spite of the fact that the immediate mitochondrial responses to FF are similar in glioblastoma cells and in NHA (11). This apparent discrepancy indicates that the observed low sensitivity of NHA to FF may involve mechanism(s), which are not directly linked to FF-induced inhibition of mitochondrial respiration. Further experiments are required to address this highly significant issue, and more specifically, to explain the difference how normal and tumor cells react to the PP1 treatment.

Conclusions

We have evaluated the anti-glioblastoma effects of four new compounds, which are modifications of a common lipid lowering drug, fenofibrate (FF). These 4 chemical modifications were developed specifically to increase the compound/s resistance to blood and tissue esterases, to improve their water solubility, tissue penetration, and ultimately anticancer potential. Our data show that the PP compounds, for example PPT, is highly effective in blocking mitochondrial respiration, and in eliminating glioblastoma cells in vitro. In comparison to the original compound, PP1 is also more stable when exposed to human blood, and is also better soluble in water. Our animal data show that PP1 accumulates in the intracranial glioblastomas following oral administration, and causes extensive necrotic damage in the intracranial tumors. Importantly, PP1-treated mice did not lose weight during the treatment, and no major pathological changes were observed in the adjacent brain tissue or in the other tested organs, with the exception of enlarged spleen, and minor necrosis and inflammation in the liver. Without wishing to be bound by theory, these results indicate the use of this new metabolic compound as a part of anti-glioblastoma therapy.

### REFERENCES CITED IN THIS EXAMPLE

1. Drukala J, Urbanska K, Wilk A, Grabacka M, Wybieralska E, Del Valle L, Madeja Z, Reiss K. ROS accumulation and IGF-IR inhibition contribute to fenofibrate/PPARalpha-mediated inhibition of glioma cell motility in vitro. Mol Cancer. 2010; 9:159. Epub 2010/06/24.
2. Grabacka M, Placha W, Plonka P M, Pajak S, Urbanska K, Laidler P, Slominski A. Inhibition of melanoma metastases by fenofibrate. Arch Dermatol Res. 2004; 296(2):54-8.
3. Grabacka M, Plonka P M, Urbanska K, Reiss K. Peroxisome proliferator-activated receptor alpha activation decreases metastatic potential of melanoma cells in vitro via down-regulation of Akt. Clin Cancer Res. 2006; 12(10):3028-36.
4. Grabacka M, Waligorski P, Zapata A, Blake D A, Wyczechowska D, Wilk A, Rutkowska M, Vashistha H, Ayyala R, Ponnusamy T, John V T, Culicchia F, Wisniewska-Becker A, Reiss K. Fenofibrate subcellular distribution as a rationale for the intracranial delivery through biodegradable carrier. Journal of physiology and pharmacology: an official journal of the Polish Physiological Society. 2015; 66(2):233-47.
5. Grabacka M M, Wilk A, Antonczyk A, Banks P, Walczyk-Tytko E, Dean M, Pierzchalska M, Reiss K. Fenofibrate Induces Ketone Body Production in Melanoma and Glioblastoma Cells. Frontiers in endocrinology. 2016; 7:5.
6. Jeansonne D, DeLuca M, Marrero L, Lassak A, Pacifici M, Wyczechowska D, Wilk A, Reiss K, Peruzzi F. Anti-tumoral effects of miR-3189-3p in glioblastoma. J Biol Chem. 2015; 290(13):8067-80.
7. Koltai T. Fenofibrate in cancer: mechanisms involved in anticancer activity. F1000Research. 2015; 4(55):1-22.
8. Panigrahy D, Kaipainen A, Huang S, Butterfield C E, Barnes C M, Fannon M, Laforme A M, Chaponis D M, Folkman J, Kieran M W. PPARalpha agonist fenofibrate suppresses tumor growth through direct and indirect angiogenesis inhibition. Proc Natl Acad Sci USA. 2008; 105(3):985-90. Epub 2008/01/18.
9. Scatena R, Bottoni P, Giardina B. Mitochondria, PPARs, and Cancer: Is Receptor-Independent Action of PPAR Agonists a Key? PPAR Res. 2008; 2008:256251.
10. Urbanska K, Pannizzo P, Grabacka M, Croul S, Del Valle L, Khalili K, Reiss K. Activation of PPARalpha inhibits IGF-I-mediated growth and survival responses in medulloblastoma cell lines. Int J Cancer. 2008; 123(5):1015-24.
11. Wilk A, Wyczechowska D, Zapata A, Dean M, Mullinax J, Marrero L, Parsons C, Peruzzi F, Culicchia F, Ochoa A, Grabacka M, Reiss K. Molecular Mechanisms of Fenofibrate-Induced Metabolic Catastrophe and Glioblastoma Cell Death. Molecular and cellular biology. 2014.
12. Kraja A T, Province M A, Straka R J, Ordovas J M, Borecki I B, Arnett D K. Fenofibrate and metabolic syndrome. Endocr Metab Immune Disord Drug Targets. 2010; 10(2):138-48. Epub 2010/04/22.
13. Cen L, Carlson B L, Pokorny J L, Mladek A C, Grogan P T, Schroeder M A, Decker P A, Anderson S K, Giannini C, Wu W, Ballman K V, Kitange G J, Sarkaria J N. Efficacy of protracted temozolomide dosing is limited in MGMT unmethylated GBM xenograft models. Neuro Oncol. 2013; 15(6):735-46.
14. Kitange G J, Carlson B L, Schroeder M A, Grogan P T, Lamont J D, Decker P A, Wu W, James C D, Sarkaria J N. Induction of MGMT expression is associated with temozolomide resistance in glioblastoma xenografts. Neuro Oncol. 2009; 11(3):281-91. doi: 10.1215/15228517-2008-090.
15. Strober W. Trypan Blue Exclusion Test of Cell Viability. Curr Protoc Immunol. 2015; 111:A3 B 1-3. Epub 2015/11/04.
16. Testra B, Mayer J. Hydrolysis in Drug and Prodrug Metabolism: Chemistry, Biochemistry, and Enzymology 2003.
17. Wang P, Choi S. Mechanism of Drug Release in Nanotherapeutic Delivery Systems. Chem Rev. 2015; 115:3388-432.
18. Salvi A, Carrupt P, Mayer J, Testra B. Esterase-Like Activity of Human Serum Albumin Toward Prodrug Esters of Nicotinic Acid. Drug Metabolism and Disposition. 1997; 25:395-8.
19. Bahar F G, Ohura K, Ogihara T, Imai T. Species difference of esterase expression and hydrolase activity in plasma. J Pharm Sci. 2012; 101(10):3979-88.
20. Najib J. Fenofibrate in the treatment of dyslipidemia: a review of the data as they relate to the new suprabioavailable tablet formulation. Clin Ther. 2002; 24(12):2022-50.
21. Redinbo M R, Potter P M. Mammalian carboxylesterases: from drug targets to protein therapeutics. Drug discovery today. 2005; 10(5):313-25.
22. Weil A, Caldwell J, Strolin-Benedetti M. The metabolism and disposition of 14C-fenofibrate in human volunteers. Drug Metab Dispos. 1990; 18(1):115-20. Epub 1990/01/01.
23. Valeur E, Bradley M. Amide bond formation: beyond the myth of coupling reagents. Chem Soc Rev. 2009; 38(2):606-31. Epub 2009/01/27.
24. Brown N. In Silico Medicinal Chemistry: Computational Methods to Support Drug Design. London: Royal Society of Chemistry; 2015.
25. Singh D. Defining desirable natural product derived anticancer drug space: optimization of molecular physicochemical properties and ADMET attributes. ADMET & DMPK. 2016; 4(2):98-113.
26. Pajouhesh H, Lutz G. Medicinal Chemical Properties of Successful Cantal Nervous System Drugs. NeuroRx. 2005; 2(4):541-53.
27. Lassak A, Dean M, Wyczechowska D, Wilk A, Marrero L, Trillo-Tinoco J, Boulares A H, Sarkaria J N, Del Valle L, Peruzzi F, Ochoa A, Reiss K. Molecular and Structural Traits of Insulin Receptor Substrate 1/LC3 Nuclear Structures and Their Role in Autophagy Control and Tumor Cell Survival. Molecular and cellular biology. 2018; 38(10). Epub 2018/02/28.
28. Nakada M, Nakada S, Demuth T, Tran N L, Hoelzinger D B, Berens M E. Molecular targets of glioma invasion. Cell Mol Life Sci. 2007; 64(4):458-78.
29. Terzis A J, Niclou S P, Rajcevic U, Danzeisen C, Bjerkvig R. Cell therapies for glioblastoma. Expert opinion on biological therapy. 2006; 6(8):739-49.
30. Ohgaki H, Kleihues P. Genetic pathways to primary and secondary glioblastoma. The American journal of pathology. 2007; 170(5):1445-53.
31. Romani M, Pistillo M P, Carosio R, Morabito A, Banelli B. Immune Checkpoints and Innovative Therapies in Glioblastoma. Frontiers in oncology. 2018; 8:464. Epub 2018/11/09.
32. Brennan C W, Verhaak R G, McKenna A, Campos B, Noushmehr H, Salama S R, Zheng S, Chakravarty D, Sanborn J Z, Berman S H, Beroukhim R, Bernard B, Wu C J, Genovese G, Shmulevich I, Barnholtz-Sloan J, Zou L, Vegesna R, Shukla S A, Ciriello G, Yung W K, Zhang W, Sougnez C, Mikkelsen T, Aldape K, Bigner D D, Van Meir E G, Prados M, Sloan A, Black K L, Eschbacher J, Finocchiaro G, Friedman W, Andrews D W, Guha A, Iacocca M, O'Neill B P, Foltz G, Myers J, Weisenberger D J, Penny R, Kucherlapati R, Perou C M, Hayes D N, Gibbs R, Marra M, Mills G B, Lander E, Spellman P, Wilson R, Sander C, Weinstein J, Meyerson M, Gabriel S, Laird P W, Haussler D, Getz G, Chin L, Network T R. The somatic genomic landscape of glioblastoma. Cell. 2013; 155(2):462-77.
33. Network T C. Corrigendum: Comprehensive genomic characterization defines human glioblastoma genes and core pathways. Nature. 2013; 494(7438):506.
34. Shen R, Mo Q, Schultz N, Seshan V E, Olshen A B, Huse J, Ladanyi M, Sander C. Integrative subtype discovery in glioblastoma using iCluster. PLoS One. 2012; 7(4):e35236. doi: 10.1371/journal.pone.0035236.
35. Champ C E, Palmer J D, Volek J S, Werner-Wasik M, Andrews D W, Evans J J, Glass J, Kim L, Shi W. Targeting metabolism with a ketogenic diet during the treatment of glioblastoma multiforme. Journal of neuro-oncology. 2014; 117(1):125-31.
36. Maroon J, Bost J, Amos A, Zuccoli G. Restricted calorie ketogenic diet for the treatment of glioblastoma multiforme. J Child Neurol. 2013; 28(8):1002-8.
37. Seyfried T N, Kiebish M A, Marsh J, Shelton L M, Huysentruyt L C, Mukherjee P. Metabolic management of brain cancer. Biochim Biophys Acta. 2011; 1807(6):577-94. doi: 10.1016/j.bbabio.2010.08.009.
38. Clendening J W, Penn L Z. Targeting tumor cell metabolism with statins. Oncogene. 2012; 31(48):4967-78. doi: 10.1038/onc.2012.6.
39. Egerod F L, Nielsen H S, Iversen L, Thorup I, Storgaard T, Oleksiewicz M B. Biomarkers for early effects of carcinogenic dual-acting PPAR agonists in rat urinary bladder urothelium in vivo. Biomarkers. 2005; 10(4):295-309.
40. Goard C A, Mather R G, Vinepal B, Clendening J W, Martirosyan A, Boutros P C, Sharom F J, Penn L Z. Differential interactions between statins and P-glycoprotein: implications for exploiting statins as anticancer agents. Int J Cancer. 2010; 127(12):2936-48. doi: 10.1002/ijc.25295.
41. Grabacka M, Reiss K. Anticancer Properties of PPARalpha-Effects on Cellular Metabolism and Inflammation. PPAR Res. 2008; 2008:930705.
42. Saidi S A, Holland C M, Charnock-Jones D S, Smith S K. In vitro and in vivo effects of the PPAR-alpha agonists fenofibrate and retinoic acid in endometrial cancer. Mol Cancer. 2006; 5:13.
43. Gardette V, Bongard V, Dallongeville J, Arveiler D, Bingham A, Ruidavets J B, Amouyel P, Haas B, Ducimetiere P, Ferrieres J. Ten-year all-cause mortality in presumably healthy subjects on lipid-lowering drugs (from the Prospective Epidemiological Study of Myocardial Infarction [PRIME] prospective cohort). Am J Cardiol. 2009; 103(3):381-6. Epub 2009/01/27.
44. Grabacka M, Pierzchalska M, Reiss K. Peroxisome Proliferator Activated Receptor alpha Ligands as Anticancer Drugs Targeting Mitochondrial Metabolism. Curr Pharm Biotechnol. 2013; 14(3):342-56.
45. Shigeto T, Yokoyama Y, Xin B, Mizunuma H. Peroxisome proliferator-activated receptor alpha and gamma ligands inhibit the growth of human ovarian cancer. Oncol Rep. 2007; 18(4):833-40. Epub 2007/09/06.
46. Wilk A, Urbanska K, Grabacka M, Mullinax J, Marcinkiewicz C, Impastato D, Estrada J J, Reiss K. Fenofibrate-induced nuclear translocation of FoxO3A triggers Bim-mediated apoptosis in glioblastoma cells in vitro. Cell Cycle. 2012; 11(14):2660-71. Epub 2012/06/27.

10.4161/cc.21015. PubMed PMID: 22732497; PMCID: 3409008.
47. Yokoyama Y, Xin B, Shigeto T, Mizunuma H. Combination of ciglitazone, a peroxisome proliferator-activated receptor gamma ligand, and cisplatin enhances the inhibition of growth of human ovarian cancers. J Cancer Res Clin Oncol. 2011; 137(8):1219-28. Epub 2011/06/18.
48. Sterba J, Valik D, Mudry P, Kepak T, Pavelka Z, Bajciova V, Zitterbart K, Kadlecova V, Mazanek P. Combined biodifferentiating and antiangiogenic oral metronomic therapy is feasible and effective in relapsed solid tumors in children: single-center pilot study. Onkologie. 2006; 29(7):308-13.
49. Zapletalova D, Andre N, Deak L, Kyr M, Bajciova V, Mudry P, Dubska L, Demlova R, Pavelka Z, Zitterbart K, Skotakova J, Husek K, Martincekova A, Mazanek P, Kepak T, Doubek M, Kutnikova L, Valik D, Sterba J. Metronomic chemotherapy with the COMBAT regimen in advanced pediatric malignancies: a multicenter experience. Oncology. 2012; 82(5):249-60.
50. Araki H, Tamada Y, Imoto S, Dunmore B, Sanders D, Humphrey S, Nagasaki M, Doi A, Nakanishi Y, Yasuda K, Tomiyasu Y, Tashiro K, Print C, Chamock-Jones D S, Kuhara S, Miyano S. Analysis of PPARalpha-dependent and PPARalpha-independent transcript regulation following fenofibrate treatment of human endothelial cells. Angiogenesis. 2009; 12(3):221-9. Epub 2009/04/10.
51. Gamerdinger M, Clement A B, Behl C. Cholesterol-like effects of selective COX inhibitors and fibrates on cellular membranes and amyloid-{beta} production. Mol Pharmacol. 2007.

52. Nadanaciva S, Dykens J A, Bernal A, Capaldi R A, Will Y. Mitochondrial impairment by PPAR agonists and statins identified via immunocaptured OXPHOS complex activities and respiration. Toxicol Appl Pharmacol. 2007; 223(3):277-87. Epub 2007/07/31.
53. Zungu M, Felix R, Essop M F. Wy-14,643 and fenofibrate inhibit mitochondrial respiration in isolated rat cardiac mitochondria. Mitochondrion. 2006; 6(6):315-22. Epub 2006/10/19.
54. Doolittle N D, Miner M E, Hall W A, Siegal T, Jerome E, Osztie E, McAllister L D, Bubalo J S, Kraemer D F, Fortin D, Nixon R, Muldoon L L, Neuwelt E A. Safety and efficacy of a multicenter study using intraarterial chemotherapy in conjunction with osmotic opening of the blood-brain barrier for the treatment of patients with malignant brain tumors. Cancer. 2000; 88(3):637-47.
55. Ikeda M, Nagashima T, Bhattacharjee A K, Kondoh T, Kohmura E, Tamaki N. Quantitative analysis of hyperosmotic and hypothermic blood-brain barrier opening. ActaNeurochir Suppl. 2003; 86:559-63.
56. Black K L, Ningaraj N S. Modulation of brain tumor capillaries for enhanced drug delivery selectively to brain tumor. Cancer Control. 2004; 11(3):165-73.
57. Yin D, Wang X, Konda B M, Ong J M, Hu J, Sacapano M R, Ko M K, Espinoza A J, Irvin D K, Shu Y, Black K L. Increase in brain tumor permeability in glioma-bearing rats with nitric oxide donors. Clin Cancer Res. 2008; 14(12):4002-9.
58. Lu D Y, Leung Y M, Huang S M, Wong K L. Bradykinin-induced cell migration and COX-2 production mediated by the bradykinin B1 receptor in glioma cells. J Cell Biochem. 2010; 110(1):141-50.
59. Montana V, Sontheimer H. Bradykinin promotes the chemotactic invasion of primary brain tumors. The Journal of neuroscience: the official journal of the Society for Neuroscience. 2011; 31(13):4858-67. doi: 10.1523/JNEUROSCI.3825-10.2011.
60. Perry J, Chambers A, Spithoff K, Laperriere N. Gliadel wafers in the treatment of malignant glioma: a systematic review. Current oncology. 2007; 14(5):189-94.
61. Saucier-Sawyer J K, Seo Y E, Gaudin A, Quijano E, Song E, Sawyer A J, Deng Y, Huttner A, Saltzman W M. Distribution of polymer nanoparticles by convection-enhanced delivery to brain tumors. Journal of controlled release: official journal of the Controlled Release Society. 2016; 232:103-12.
62. Carlson B L, Pokorny J L, Schroeder M A, Sarkaria J N. Establishment, maintenance and in vitro and in vivo applications of primary human glioblastoma multiforme (GBM) xenograft models for translational biology studies and drug discovery. Current protocols in pharmacology/editorial board, SJ Enna. 2011; Chapter 14:Unit 14 6.

### Example 3

Exploring Anticancer Activity of Structurally Modified Benzylphenoxyacetamide (BPA). I: Synthesis Strategies and Computational Analyses of Substituted BPA Variants with High Anti-Glioblastoma Potential

Abstract

Structural variations of the benzylphenoxy acetamide (BPA) molecular skeleton were explored as a viable starting point for designing new anti-glioblastoma drug candidates. Hand-to-hand computational evaluation, chemical modifications, and cell viability testing were performed to explore the importance of some of the structural properties in order to generate, retain, and improve desired anti-glioboastoma characteristics. It was demonstrated that several structural features are required to retain the anti-glioblastoma activity, including a carbonyl group of the benzophenone moiety, as well as 4′-chloro and 2,2-dimethy substituents. In addition, the structure of the amide moiety can be modified in such a way that desirable anti-glioblastoma and physical properties can be improved. Via these structural modifications, more than 50 compounds were prepared and tested for anti-glioblastoma activity. Some of the compounds, for example HR28, HR32, HR37, HR40(PP1), and HR46, have been determined to have desirable physical and biological properties with potential to become anti-glioblastoma drugs.

Introduction

Glioblastoma is the most aggressive and prevalent malignancy of the central nervous system (CNS), with a median patient survival rate of about 15 months¹⁻⁴. Surprisingly, the most effective way to increase survival of glioblastoma patients is still extensive surgical resection. However in many instances, this approach is not feasible due to the tumor's location and its infiltration of highly specialized brain areas⁵. The current standard of care therapies include maximal surgical resection, followed by radiotherapy plus concomitant and maintenance temozolomide (TMZ), which is one of the few anticancer drugs capable of crossing the blood brain barrier (BBB)⁶. Unfortunately, TMZ-treated tumors develop TMZ-resistance, and recurrent glioblastomas are practically incurable⁷⁻⁹. Moreover, numerous clinical trials targeting a variety of glioblastoma-specific pathways, as well as, those testing immune checkpoint inhibitors, have been implemented, but have failed to produce a positive outcome in glioblastoma patients^{2,10,11}.

Therapeutic strategies that include TMZ in combination with other drugs have been also explored. For instance, glioblastomas are characterized by exaggerated lipogenesis, enhanced LDL and cholesterol uptake, and extensive phagocytosis and exosome formation. All of these processes require high cholesterol metabolism and uptake for a continuous biogenesis of cellular membranes. Therefore, a combination of cholesterol lowering drugs with TMZ might be a good approach for glioblastoma treatment¹². One lipid-lowering drug that has attracted attention as a candidate for an anticancer regimen is fenofibrate (FF) 13-18 In recent studies, it was demonstrated that 50 μM FF has a strong anticancer activity with low systemic toxicity^{13,16,19} We have previously reported that fenofibrate, in its unprocessed form (ester), blocks mitochondrial respiration, resulting in a severe energy deficit, followed by extensive glioblastoma cell death¹⁹. However, fenofibrate does not cross the BBB, and is quickly processed by the blood and tissue esterases to form fenofibric acid (FFA). This acid functions as a potent peroxisome proliferator activated receptor a (PPARa) agonist, however, it is no longer effective in triggering tumor cell death^{19,20}.

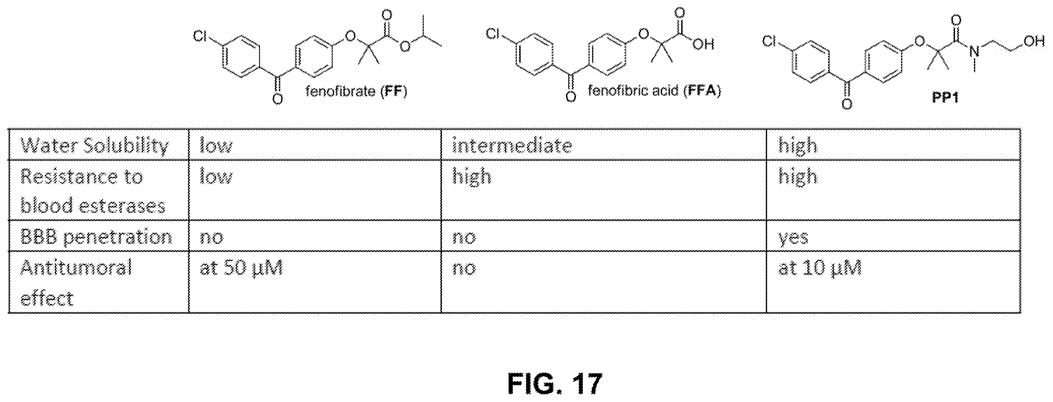

We have made several chemical modifications to the FF structure in order to improve the prospective anticancer drug stability, water solubility, tissue penetration, and ultimately, the anti-glioblastoma efficacy. One of the compounds, PP1 (FIG. 17), triggered extensive glioblastoma cell death in vitro at concentrations almost 5-fold lower than FF²¹. Similar to FF, PP1 inhibited mitochondrial respiration, and demonstrated improved water solubility, BBB penetration, and resistance to blood esterases²¹. However, PP1 accumulation in the brain tumor tissue (following oral administration) may not be sufficient to exert a highly effective anti-glioblastoma effect in vivo. HPLC-measured concentrations of PP1 in the brain tumor tissues, from orally administered PP1 in mice, varied between 5 and 6 μM. Since highly effective tumor cell death occurs at 10 μM of PP1²¹, we further explored the anti-tumoral contribution of the specific chemical moieties in the FF and PP1 structures, to improve the compound anticancer efficacy and its ability for more effective accumulation within the brain tumor tissue. As a result, 50 additional compounds were generated and analyzed in this study.

Design of the Molecular Target

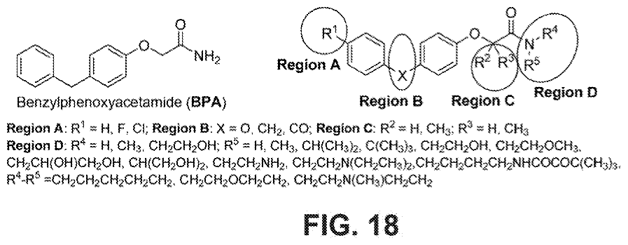

The basic molecular skeleton of FF contains a benzylphenoxy acetate structural arrangement (FIG. 17). Although FF shows promising anti-glioblastoma activity at 50 μM, this compound is an isopropyl ester that is promptly hydrolyzed into FFA by blood and tissue esterases²⁰. An additional disadvantage of FF is that it has low water solubility, and a relatively high concentration is required for its antitumoral activity (50 μM)¹⁹. We have selected benzylphenoxyacetamide (BPA) molecular skeleton as a basis for designing new, more potent anti-glioblastoma compounds. Four specific regions of the BPA were subsequently designated for chemical modification in order to determine how the nature of substitutions alter anti-glioblastoma activity. Structural variations to the chosen regions include exchange of halogens (region A); addition or removal of oxygen, methylene, carbonyl (region B); addition or removal of hydrogens, one methyl, or two methyl groups (region C); and replacement, removal or addition of one alkyl, two alkyl, one hydroxyalkyl, two hydroxyalkyl, and alkyl with primary, secondary, or tertiary group (region D). General structures that combine all these alterations are presented in FIG. 18.

Preparation Methods

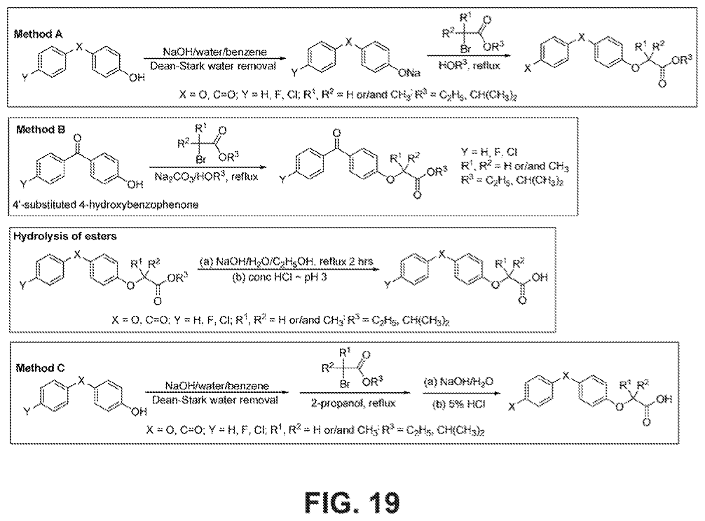

All preparations (FIG. 19 Methods A-C) were started with either substituted phenoxyphenols or benzoylphenols. The phenols were first converted into their corresponding sodium salts. Due to the different acidity of these two groups of compounds (pKa of about 10 for phenoxyphenols and 8 for benzoylphenols), two different bases were used. Sodium hydroxide in water/benzene can be used for preparation of sodium salts of both groups of phenols (FIG. 19 Method A). Sodium hydroxide was a sufficiently strong base (pKa ˜15.7) to deprotonate the phenoxyphenols, and the Deen-Stark distilling receiver was used to azeotropically remove water. Therefore, a dry white powdery sodium phenoxide resulted and was used in a second step: nucleophilic substitution of the corresponding 2-bromoalkanoic esters. Isolated yields were almost quantitative. However, for more acidic benzoylphenols, one-pot synthesis in alcohol with anhydrous sodium carbonate can be used as well (FIG. 19 Method B). This approach requires longer time but is simpler, safer, more economical and it is the method of choice for the preparation of benzoylphenoxyethanoic acid. However, preparation of phenoxyethanoic acids can also be accomplished in one-pot synthesis without isolation of the intermediate ester (FIG. 19 Method C). In this method, the corresponding phenol is first converted into its sodium salt with sodium hydroxide via Dean-Stark distillation with molecular sieves²², followed by a reaction with 2-bromaethanoic ester in isopropanol, and finally a hydrolysis step with aqueous sodium hydroxide in isopropanol. These products are easily purified by an acid-base extraction. This is the preferred method for direct preparation of phenoxyethanoic acids. Isolated yields are between 85 to 95%.

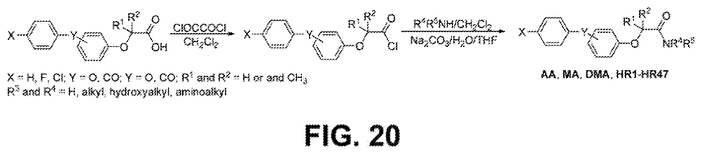

The starting point for the preparation of each BPA modification in this study is the benzylphenoxyacetic acid derivative. For amides, although there is a plethora of synthetic methods for transformation of acids into amides, many of these methods cannot be successfully applied for preparation of all the amides included in this study. For instance, employing a well-developed amide preparation that utilizes DCC and EDC based activation agents²³ for direct carboxylic acid conversion to amide cannot be used in the case of 2,2-dimethylbenzylphenoxyacetic acid. This is due to a combination of steric hindrance and lower reactivity of the activated carboxylic acid. However, when the corresponding carboxylic acid chloride is used, almost quantitative conversion of carboxylic acid into amide is achieved (FIG. 20).

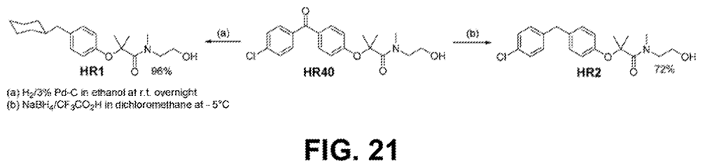

Reduced forms of the studied amides were prepared by selective reduction (FIG. 21). Catalytic hydrogenation, such as that performed with 3% palladium (Pd) on carbon, removes halogens, reduces the carbonyl group to a methylene group, and reduces the aromatic ring that contains chlorine. The reaction is carried out at room temperature and hydrogen at atmospheric pressure. The isolated yield is almost quantitative. Only one product was prepared in this way by reducing HR40 (PP1) to HR1 (FIG. 21). Selective reduction of the carbonyl group for preparation of BPA was accomplished by using a combination of sodium borohydride and trifloroacidic acid at 5° C. In this way, HR2 was prepared by the reduction of HR40 (FIG. 21). In this case, the product had to be separated from the starting material by chromatography, so isolated yield was only 72%.

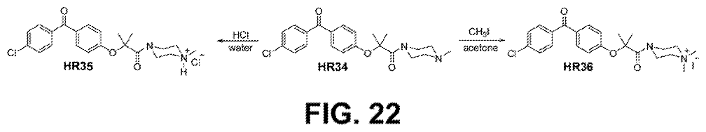

To further increase the water solubility of basic BPA, ammonium salts were prepared. Hydrochloric salts were prepared by simple mixing of the corresponding BPA with concentrated hydrochloric acid followed by water evaporation. Then, alkylation of basic BPA was performed with acetone as the solvent and methyl iodide as the alkylating reagent. The product (HR36) crystalized directly from the reaction mixture (FIG. 22).

Results and Discussion

As mentioned above, it was demonstrated that FF possesses anti-glioblastoma activity. However, there are several FF properties that make it impractical for anticancer treatment: low water solubility, fast hydrolysis into FFA, and a required relatively high therapeutic concentration^{19,20} In our previous study, we reported that some amide derivatives of FF, including PP1, were more potent in eliminating glioblastoma cells than FF²¹. These amides belong to the large family of BPA²⁴.

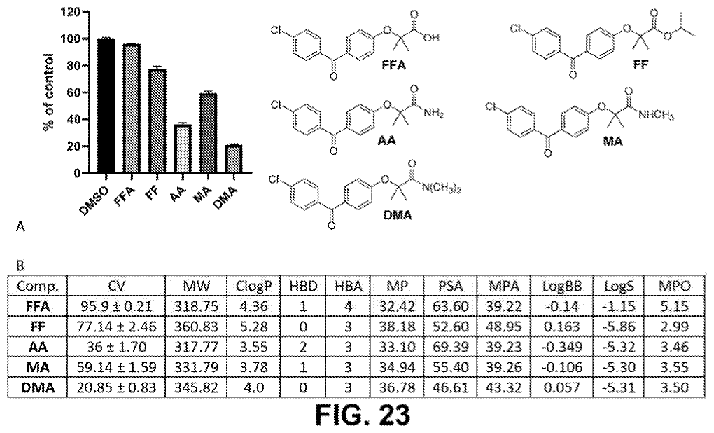

One fundamental challenge for the design of CNS penetrant drugs is the need to cross the blood-brain barrier (BBB). But, BBB-permeable compounds form a very small subset of oral drugs currently in existence, and experimental models for testing BBB penetration are quite complex. Therefore, an independent indicator of the BBB penetration is needed for the initial screening and selection of large number of compounds (BPA variants) to evaluate their potential for reaching intracranial tumor site at therapeutically relevant concentrations. Therefore, prior to the preparation of all BPA variant compounds in this study, we performed extensive molecular modeling to describe their physicochemical properties. Cell viability (CV) assay was then performed using LN229 human glioblastoma cell line, and the cells were treated with BPA variants at 25 M for 72 hours. The results of these computational and cell viability testing are outlined in FIGS. 23-32, below.

One currently accepted way to define physicochemical properties is by using a weighted scoring approach, known as the Central Nervous System—Multiparameter Optimization (CNS-MPO)^{25,26} The CNS-MPO algorithm uses a weighted scoring function that assesses 6 key physicochemical properties (c log P, c log D, MW, TPSA, HBD, and pKa) that indicate relative BBB penetration. The CNS-MPO scale is between 0 and 6.0, with scores ≥4.0 widely used as a cut-off to select compounds for hit CNS therapeutic drug discovery programs²⁶. The validation of this approach utilized a library of 616 compounds to evaluate the experimental distribution of the computed parameters incorporated into CNS-MPO scores^{25,26}. It was found that CNS-MPO scores of 1-2 (0%), 2-3 (11.6%), 3-4 (40.8%), 4-5 (53.8%) and 5-6 (81.1%) increase the probability of drugs to be found in the brain²⁷.

In addition, parameters that are routinely used for Quantitative Structure Activity Relationship (QSAR) study are molecular polarizability (MP), minimal molecular projection area (MPA), and water solubility (Log S). These parameters are not incorporated into the CNS-MPO score, however they are also expected as possible contributing factors in BBB penetration. Therefore, in the associated figures herein, these additional parameters were defined and calculated for each BPA variant compound. MP of a molecule characterizes the capability of its electronic system to be distorted by the external field, and it plays an important role in modeling many molecular properties and biological activities²⁸. Without wishing to be bound by theory, an MP between 30-40 is optimal for a molecule to bind to a biotarget²⁹. Minimal projected area (MPA) is also very important for drug transport and ultimately for drug activity. For instance, in recent studies by Cha, Müller, and Pos, a distinct phenotypical pattern of drug recognition and transport for the G616N variant was reported, indicating that drug substrates with MPA of >70 Å² are less well transported than other substrates³⁰. Finally, Log S of −4.5 and greater are indicators of acceptable water solubility³¹.

The BBB permeation propensity of all studied compounds is indicated by the decimal logarithm of brain to-plasma concentration ratio (log BB) value, which is derived from the modified Clark's equation: log BB=0.152 C log P −0.0148PSA+0.139 30. This parameter was also calculated and is listed in the tables. It has been shown that chemical compounds with log BB>0.3 readily cross the BBB, while those with log BB<−1.0 are poorly distributed to the brain³². Finally, the rate of passive diffusion is inversely proportionate to the square root of molecular size (Graham's law³³), which is also included in our compound analysis.

Minimal projected area (MPA, FIG. 23) is also very important for drug transport and ultimately for drug activity. For instance, in recent studies by Cha, Müller, and Pos, a distinct phenotypical pattern of drug recognition and transport for the G616N variant was reported, indicating that drug substrates with MPA of >70 Å² are less well transported than other substrates³⁰. All estimated MPA for molecules presented in FIG. 23 are between 35 and 50 Å²; therefore, based on their size alone they should be capable of crossing the cell membrane³⁴. Therefore, MPA results presented in FIG. 23 confirm that the simple amides of FFA are viable structural motifs for exploring and possibly improving the anti-glioblastoma activity of FF.

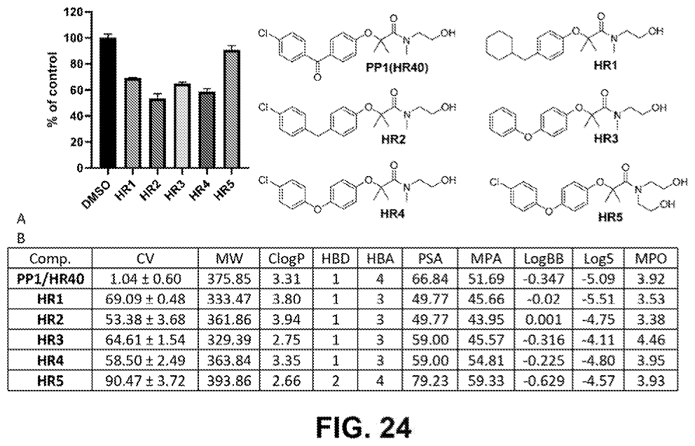

Our previously reported drug candidate PP1 (HR40) has an excellent activity against glioblastoma tumor cells²¹, therefore, we are using this drug candidate as a standard to evaluate the structural modifications made on the BPA skeleton. Therefore, we investigated first the importance of the second aromatic ring in BPA, the conjugation of the carbonyl group, and the presence of a chlorine atom. All computed parameters (FIG. 24) indicate that these compounds will have moderately desirable physical properties for BBB penetration. C log Ps values are in the middle of the desirable range 2.0 to 5.0, and MPO values are between 3.5 and 4.5, therefore both suggest a possibility for these compounds to accumulate in the CNS (FIG. 24). However, according to the calculated Log BB for these compounds, a low brain penetration ability could be also expected. The cell viability data at M for these 5 compounds (HR1-HR5) showed moderate to low anti-glioblastoma activity (FIG. 24).

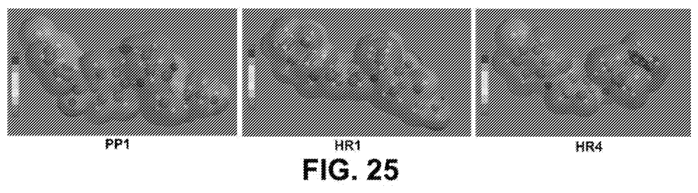

Replacing the 4-chlorobenzoyl moiety of PP1 with a cyclohexylmethyl generates a new drug candidate HR1. However, this modification results in a decrease of anti-glioblastoma activity (FIG. 24). This is a somewhat drastic structural change, resulting in conformational and noticeable structural electrostatic potential surface changes (FIG. 25) 35. Lipophilicity is higher and molecular polar surface area is substantially lower (from 66.84 for PP1 to 49.77 for HR1 (FIG. 24B). However, if the carbonyl group of the benzophenone moiety is replaced with a methylene group (HR2), a change in the electrostatic potential in the middle of the molecule results in a less effective compound (CV=53.4%) compared to PP1 (CV=1%), but slightly better than HR1 (CV=69.09%) (FIG. 24). If the presence of a polar group in the middle of the molecule is important, then replacement of the carbonyl group of the benzophenone moiety of PP1 with an ether group (oxygen atom) should give a new drug candidate, HR4, with similar activity to PP1. However, if the carbonyl group is replaced with a methylene group or oxygen atom, the newly formed drug candidate have only moderate anti-glioblastoma activity (CV=58.5%) (FIG. 24). Due to these results, the presence of a second aromatic ring, the carbonyl linker, and chlorine are all important for retaining anti-glioblastoma activity of PP1 (FIG. 24).

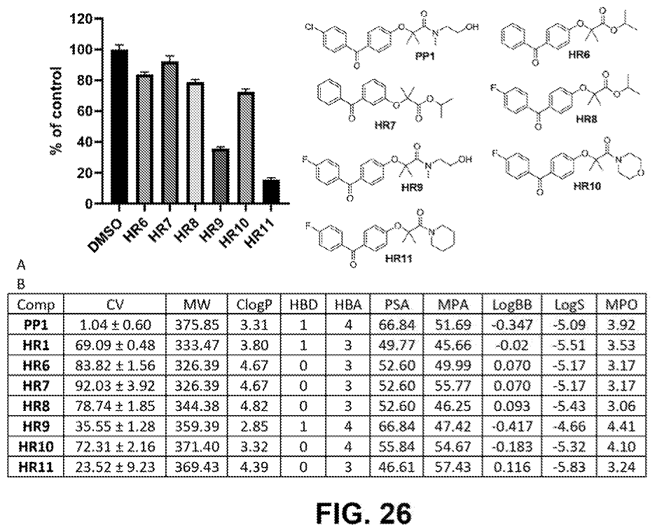

In addition, it is also important to assess the significance of the halogen atoms on the BPA skeleton. Substituting hydrogen by fluorine substantially changes molecular polarizability (MP) and lipophilicity, and increases binding affinity to targeted proteins³⁶. Also, halogen bonding is stronger between chloro-aryls and carbonyl compounds, than between corresponding fluoro-aryls [33, 34]. Therefore, the computed data, as well as, the cell viability data of modified variants of PP1 in which the chlorine atom was replaced with fluorine (HR8-HR11) were collected (FIG. 26). Although, the ester of fluoro-FF (HR8) has virtually the same activity (CV) as FF, other esters (HR6, HR7) are virtually inactive at the same concentration (25 μM) (FIG. 26). So, further exploration of the anti-glioblastoma activity for ester derivatives of FF is not necessary.

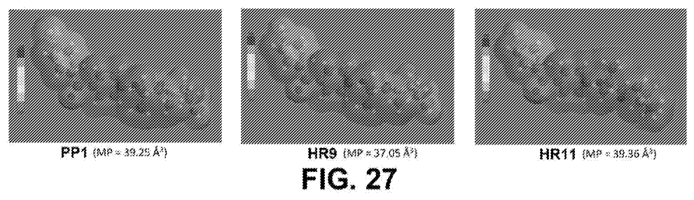

Fluoro-PP1 (HR9) has lower potency than PP1, and therefore, halogen bonding appears to be very important for anti-glioblastoma activity in BPAs (FIG. 26). The order of activity for the tested amides is presented in FIG. 26 is PP1>HR11>HR9. However, the computed physicochemical parameters do not include an indicator of the effect of halogen bonding, which therefore, underestimates the contribution of the chlorine bonding and wrongly predicts that PP1 should be less active than the corresponding fluoro-isomer HR9. Electrostatic potential maps for PP1 and HR9 are very similar, therefore they are excepted too have similar anti-glioblastoma activity in terms of molecular potential. However, molecular polarizability (39.25 Å³ versus 37.05 Å³) is quite different indicating different binding capabilities. Surprisingly, the molecular polarizability of HR11 (39.36 Å³) is quite close to that of PP1 (FIG. 27), indicating that HR11 might have a stronger binding capability to the targeted molecule than HR9, which is reflected by a slightly higher biological activity (FIG. 26).

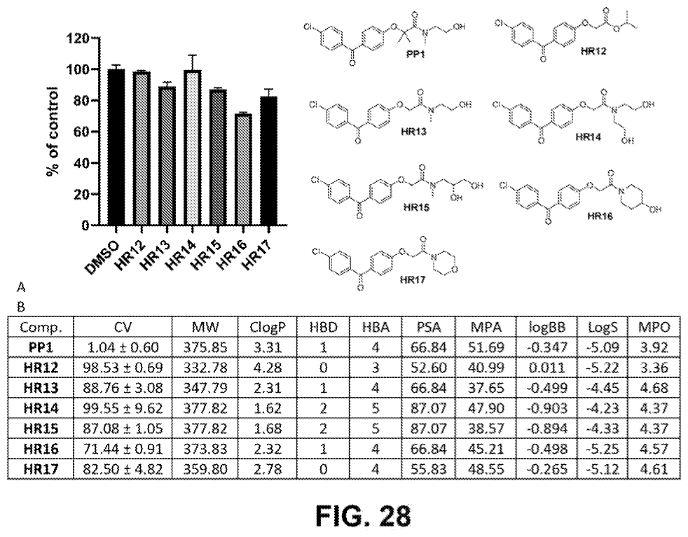

The BPA structure was further modified in order to explore the importance of two methyl groups in the alpha position (FIG. 18; region C). For non-methylated (unsubstituted alpha position) phenoxyamides, the physicochemical parameters and biological activity results are presented first in FIG. 28. All computed compounds, here, have acceptable computed polar surface area (PSA) and minimal projected area (MPA), which indicate that they can penetrate the cell membrane^{25,26,30} Estimated CNS-MPO for amides HR13-HR17 are all more desirable than for PP1 (FIG. 28). Based on this data, one would expect these compounds to have at least comparable biological activity to, if not better than, PP1. However, they are almost all inactive at the tested 25 μM concentration (FIG. 28). It is obvious that the presence of at least one methyl group is crucial for retaining activity.

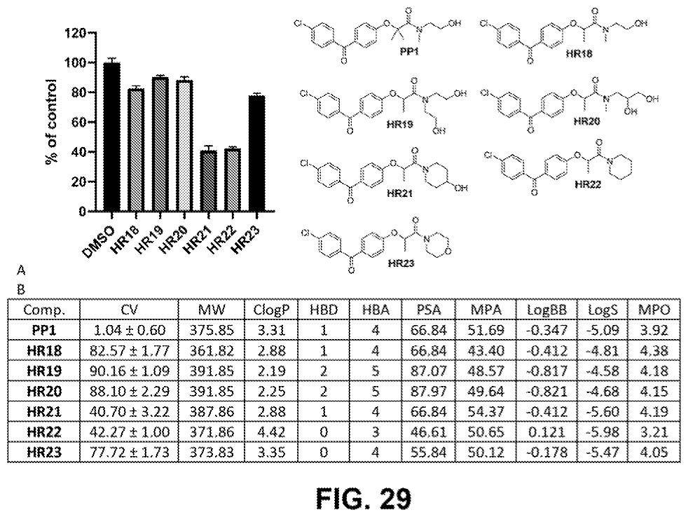

The computed physical properties for monomethylated amides (shown in FIG. 29) are closer to the estimated physical properties of PP1, but their biological activity at 25 μM is still very low (FIG. 29). It is now clear that two methyl groups are essential for retaining anti-glioblastoma activity.

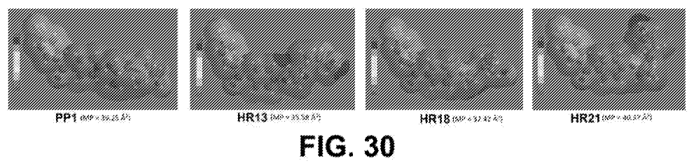

Comparison of electrostatic potential for di-, mono-, and non-methylated compounds (HR13, HR18, HR21) suggests that a large positive area in the molecule is lost by removing the methyl groups (FIG. 30). However, when a larger non-polar amide moiety is introduced, such as in the creation of HR21 (FIG. 30), a new positive electrostatic potential surface is generated that partially offsets the nonexistence of the methyl group. This is also indicated by a computed molecular polarizability (MP) that is slightly higher than that of PP1, resulting in modest activity (about 60% cell death) for both HR21 and HR22 compared to PP1 (FIG. 29). Therefore, in spite of the slight offset of the electrostatic potential maps, it seems apparent that two methyl groups in the alpha position of BPA are essential for retaining anti-glioblastoma activity. Therefore, the structural configuration necessary for anti-glioblastoma activity is equal to AA (FIG. 23).

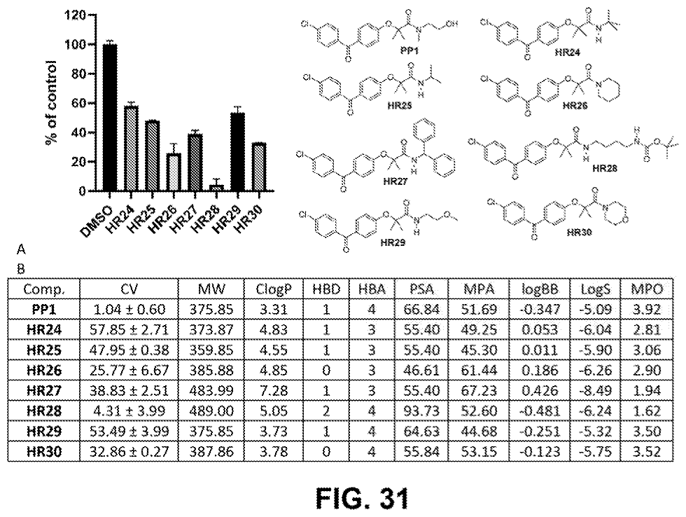

After establishing the importance of the 2-(4-chlorobenzoyl)phenoxy-2,2-dimethylacetamide (AA) structural skeleton for anti-glioblastoma activity, it is essential to assess the nature of the amide moiety (FIG. 18 region C). Three groups of AA variants were designed, prepared and tested. The first group of compounds contained an amide moiety with neutral pH (FIG. 31) in order to establish the size and structural branching on the amide part of the molecule. Again, the physicochemical parameters and biological activity data were compared to our good drug candidate PP1. The computed MPAs are acceptable (40-60 Å²) and suggest that based on this property alone all of these compounds should have good cell permeability. On the other hand, based on the estimated PSA and CNS-MPO data, HR28 should be expected to have a lower probability of penetrating into the cell, and possibly lower accumulation in the CNS. This is in spite of the fact that HR28 is very potent in eliminating glioblastoma cells in vitro (CV=4.31%).

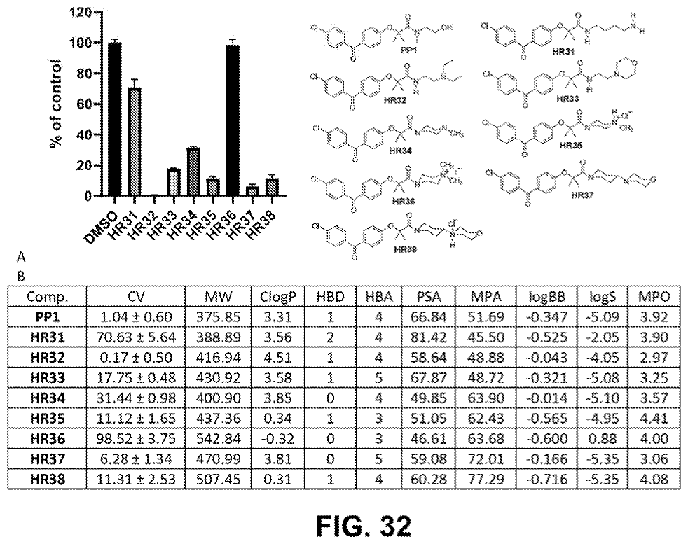

Next, amides with a basic amide moiety are presented in FIG. 32. For these compounds, cell permeability is strongly associated with the pH of the media. In acidic media, they are protonated and very hydrophilic. Additionally, they can be easily alkylated, which in turn can modify their polarity. All cell testing was done at physiological pH (pH=7.4); therefore, the basic drug candidates were not protonated. Amide HR31 has two nitrogen atoms separated by four methylene groups. One nitrogen is part of an amide bond and other is part of a primary amine. This compound has a CV value similar to that of 25 μM FF 19 However, when the carbon chain is shortened between two nitrogens by two carbon atoms and the primary amine is transferred to a tertiary amine with two ethyl groups, a new drug candidate, HR32, is formed, with the highest anti-glioblastoma activity (CV=0.17), and is more potent than PP1 (FIG. 32). Almost all computed parameters suggest that this is a good candidate (HR32); however, the CNS-MPO estimated value is relatively low (2.97), suggesting that it may have a low probability of accumulating in the CNS. However, both the computed log BB and log S for this compound suggest that this, in fact, would be a good drug candidate. The other amides in this group (HR33-HR38) with the exception of HR36 have a good anti-glioblastoma activity, including the hydrochloric salt HR35, which in addition has a more promising CNS-MPO (4.41). In fact, HR35 is more potent than its free base HR34 (FIG. 32). One could argue that this is due to better initial solubility upon the drug's administration of this hydrochloric salt. However, in cell culture environment, HR35 should be deprotonated and become the free base HR34 (FIG. 32). This is totally reversed in the case of HR37 and its hydrochloride salt HR38. The free base, HR37, is more potent than its hydrochloric salt (HR38).

In the case of the alkylated drug candidates, such as methylated HR34, the activity is substantially diminished because it cannot be deprotonated. If one examines the computed parameters for HR36, it is apparent that the compound is very hydrophilic (C log P=−0.31; log S=0.88) and has a very low estimated BBB (log BB=−0.6). Surprisingly the CNS-MPO of HR36 is 4.0, which would suggest that this compound should be capable of accumulating in CNS, however it is completely inactive at 25 μM (CV=98%) (FIG. 32). In conclusion, the best candidate from this group of compounds (HR31-HR38) is HR32, which more potent than PP1. In addition, HGR35 and HR37 are also good candidates mostly because of their relatively high CNS-MPO and low CV values. In contrast, HR36, which is permanently positively charged, completely loses its anti-glioblastoma activity.

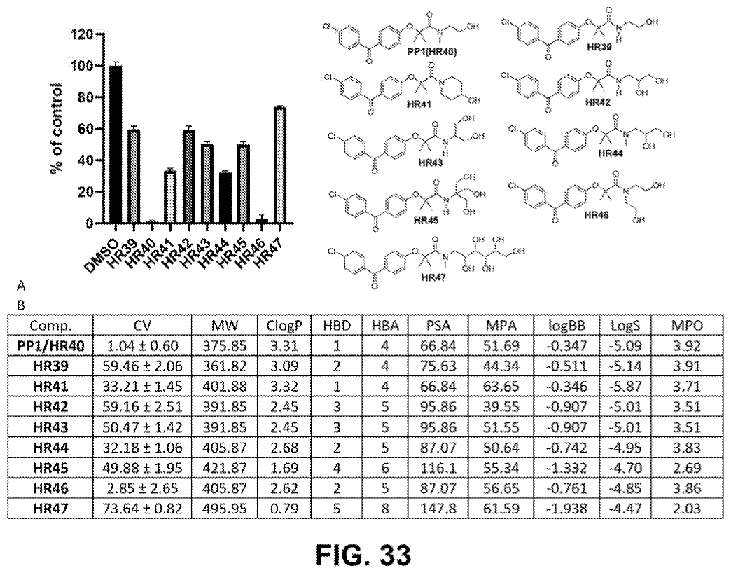

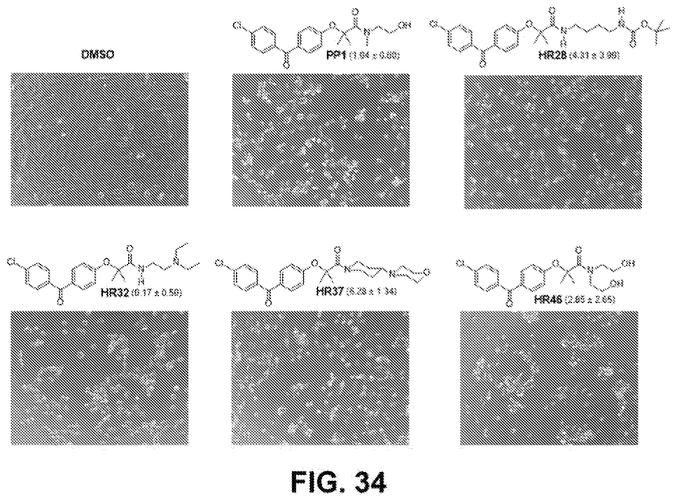

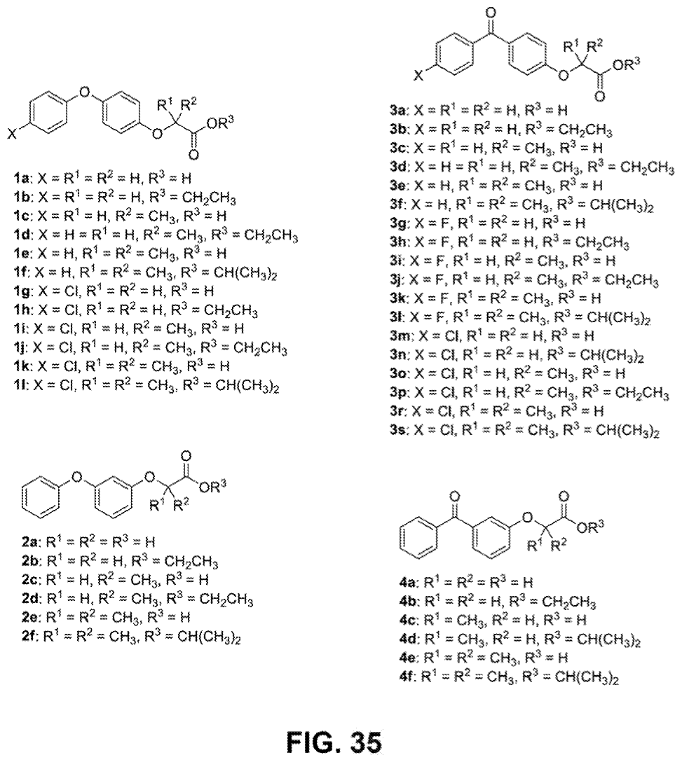

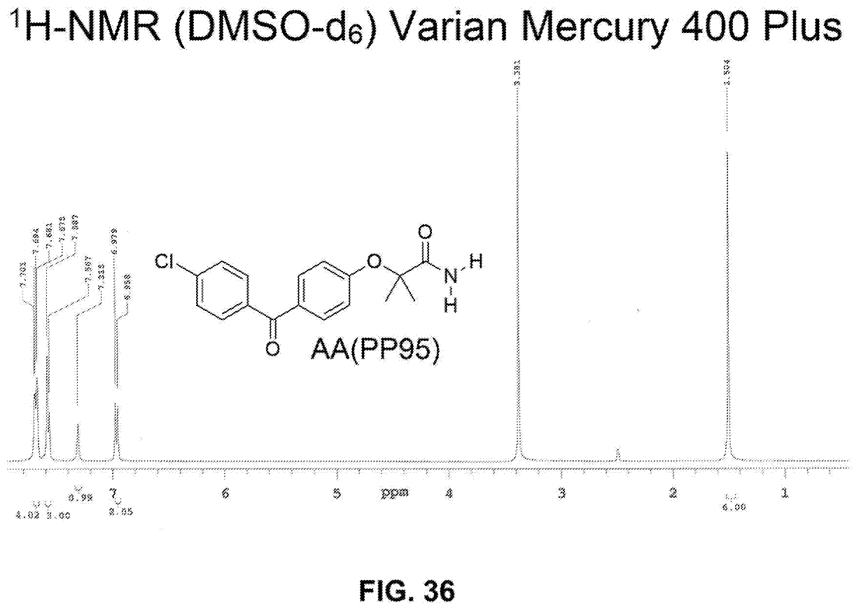

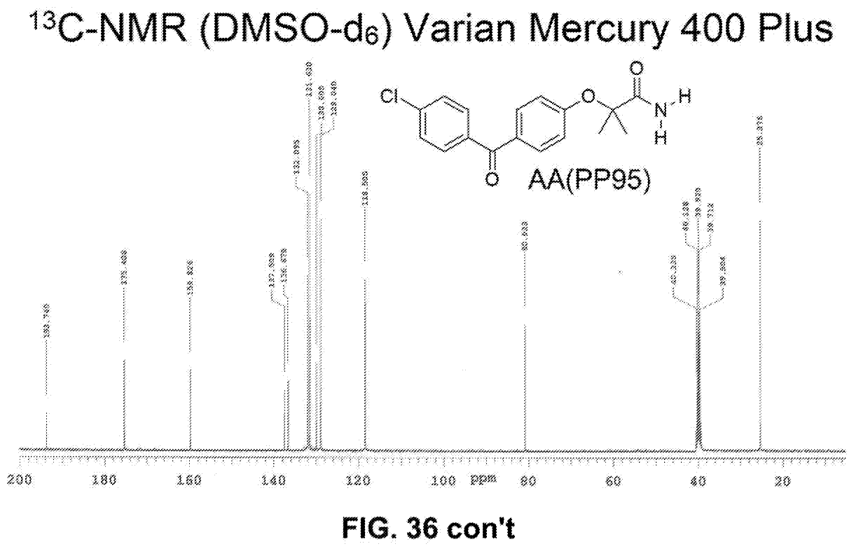

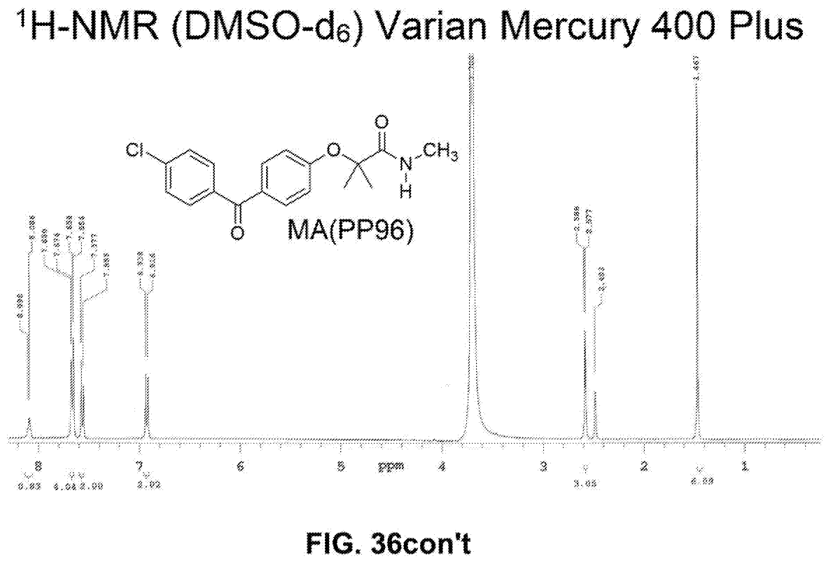

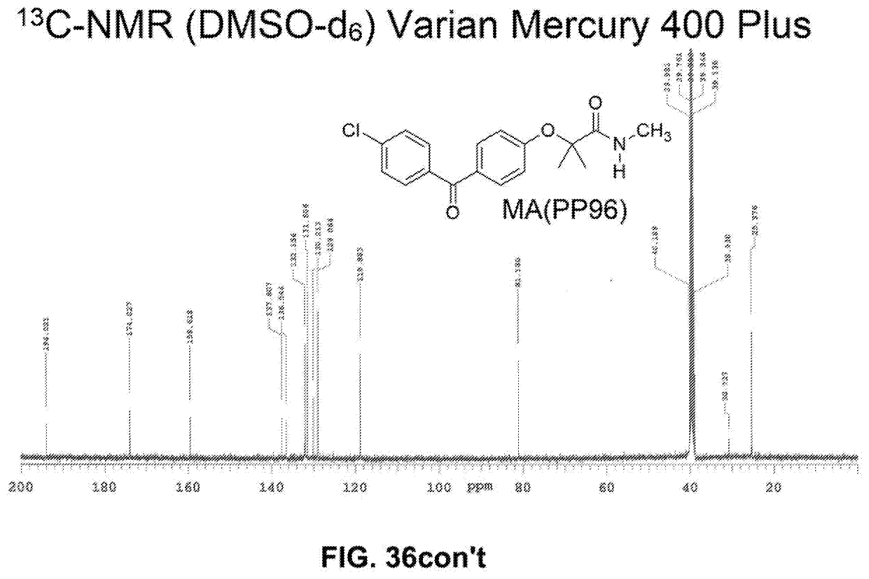

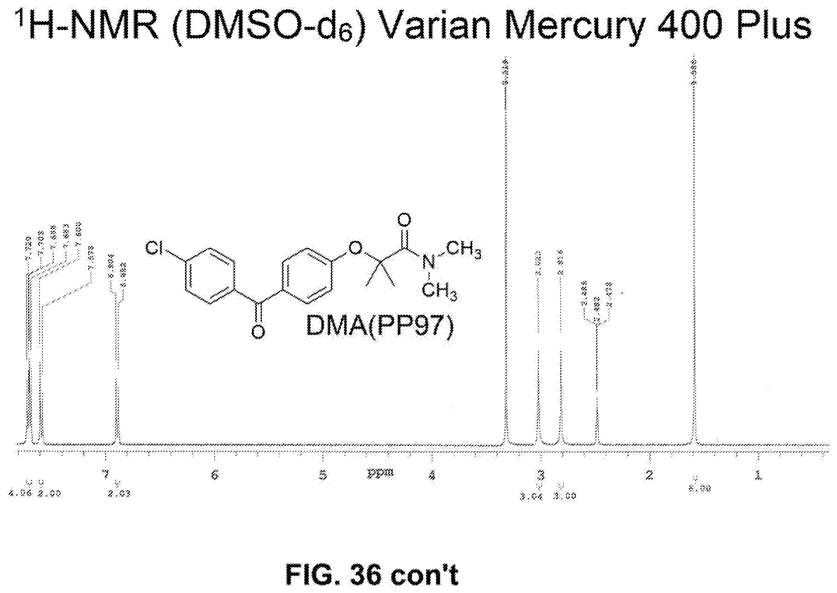

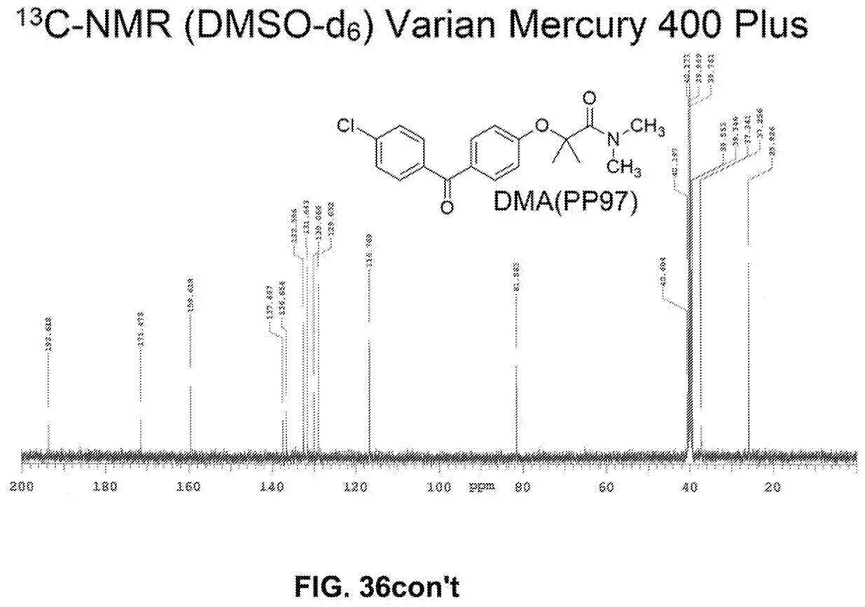

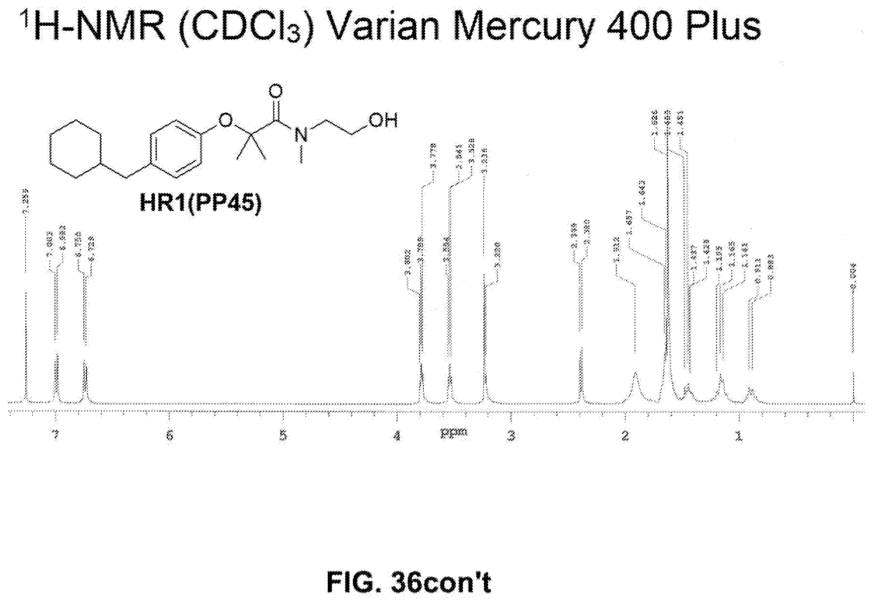

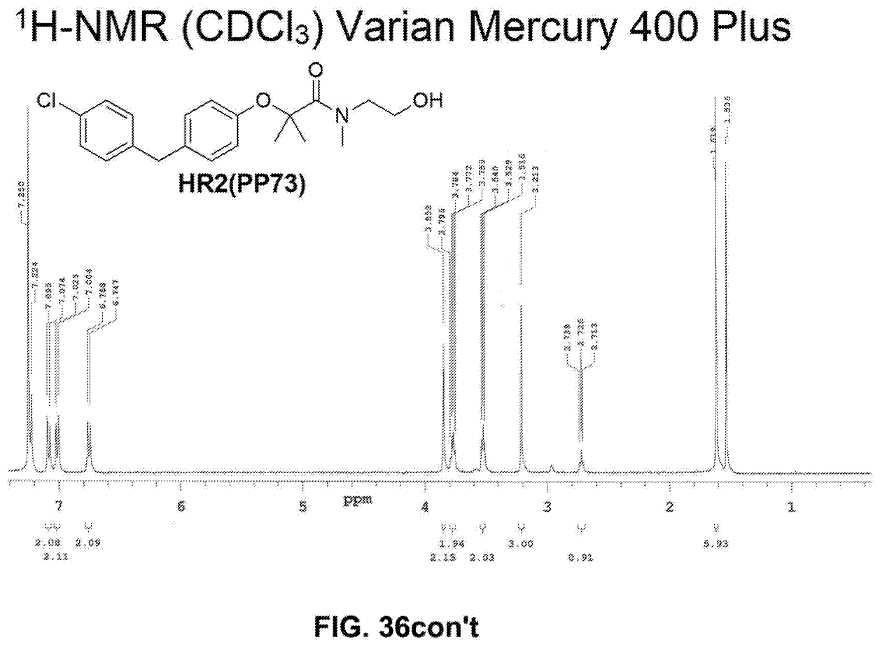

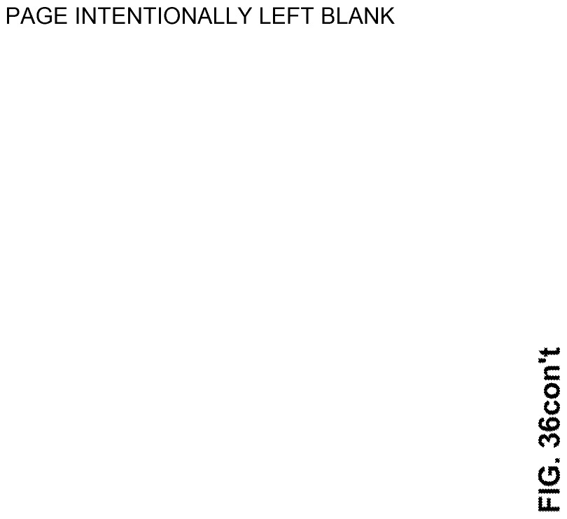

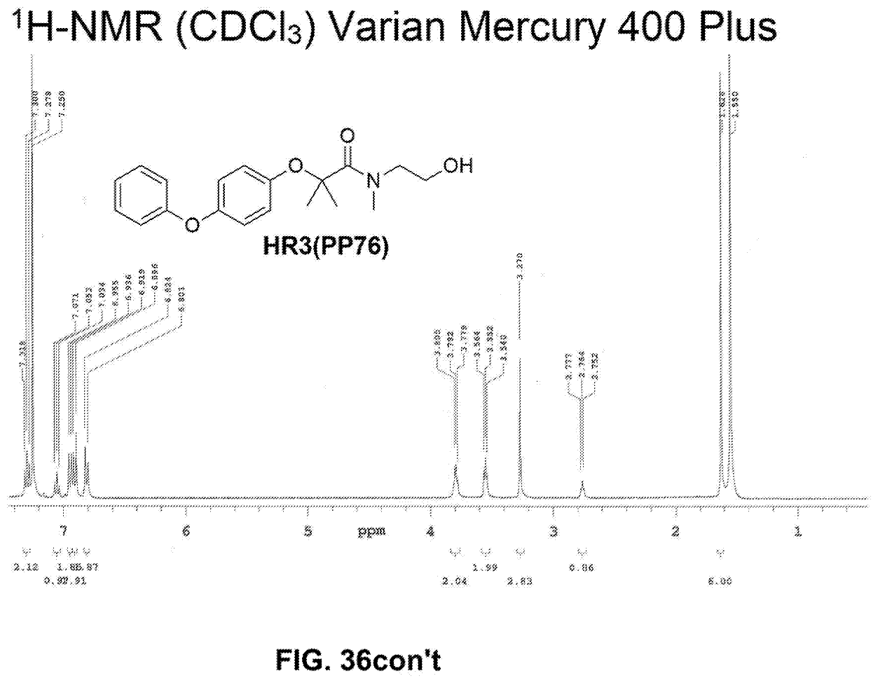

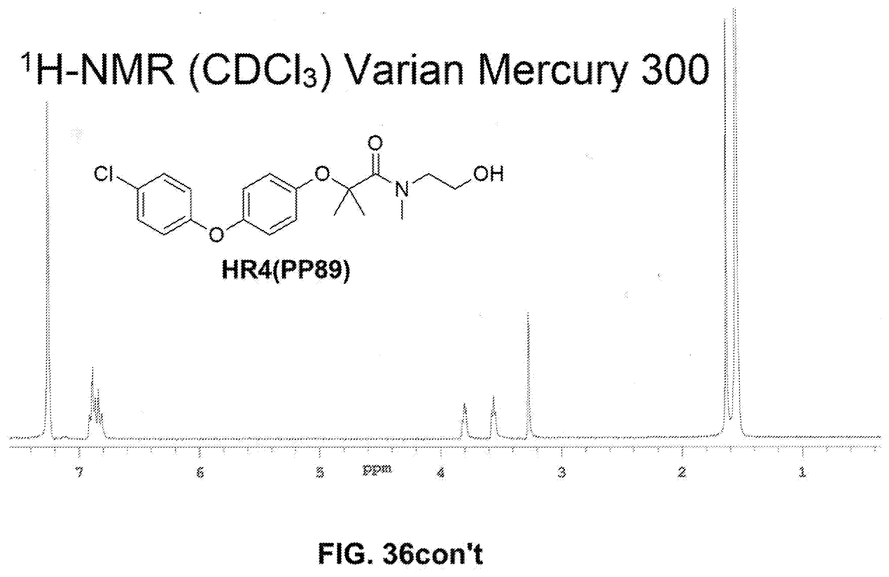

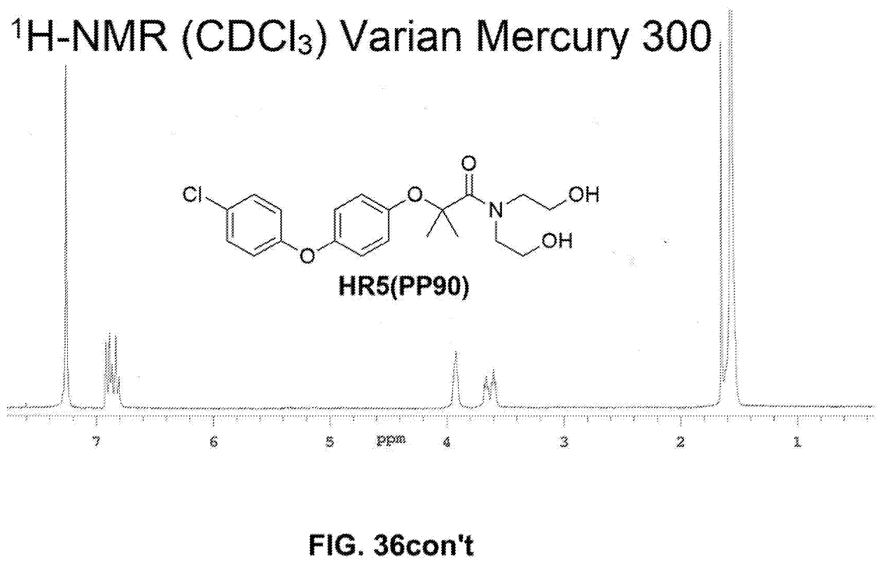

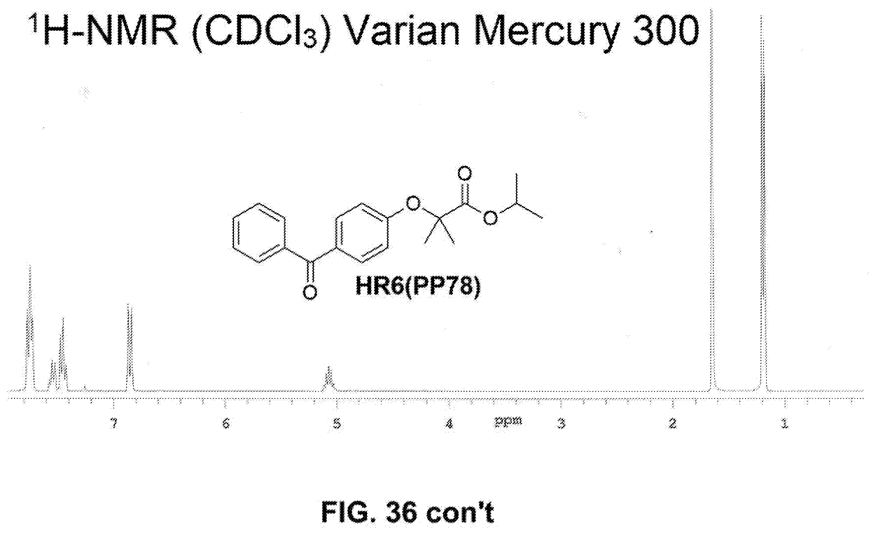

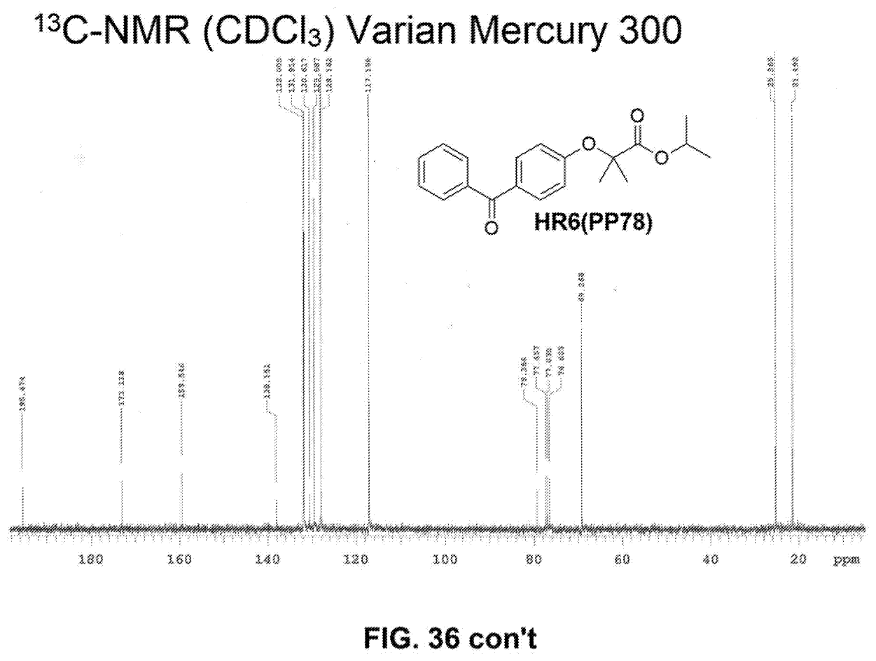

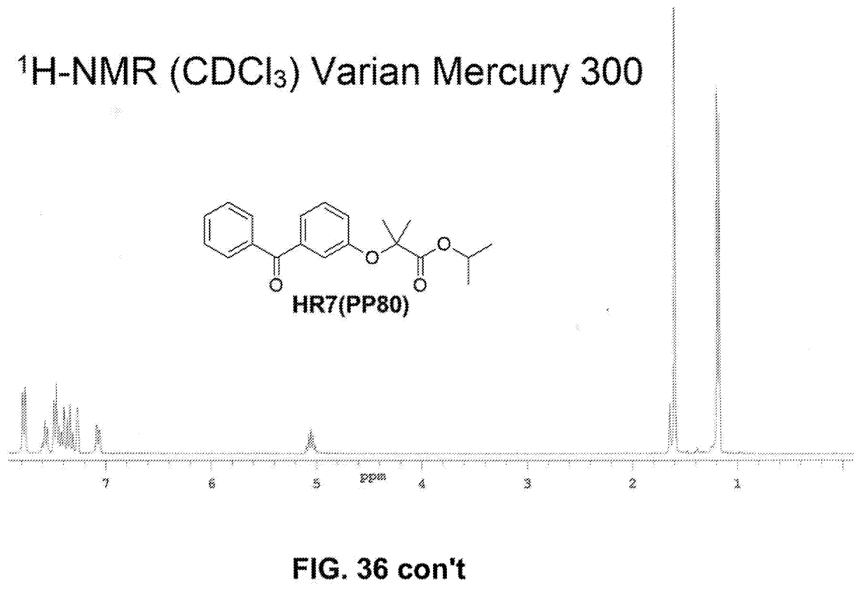

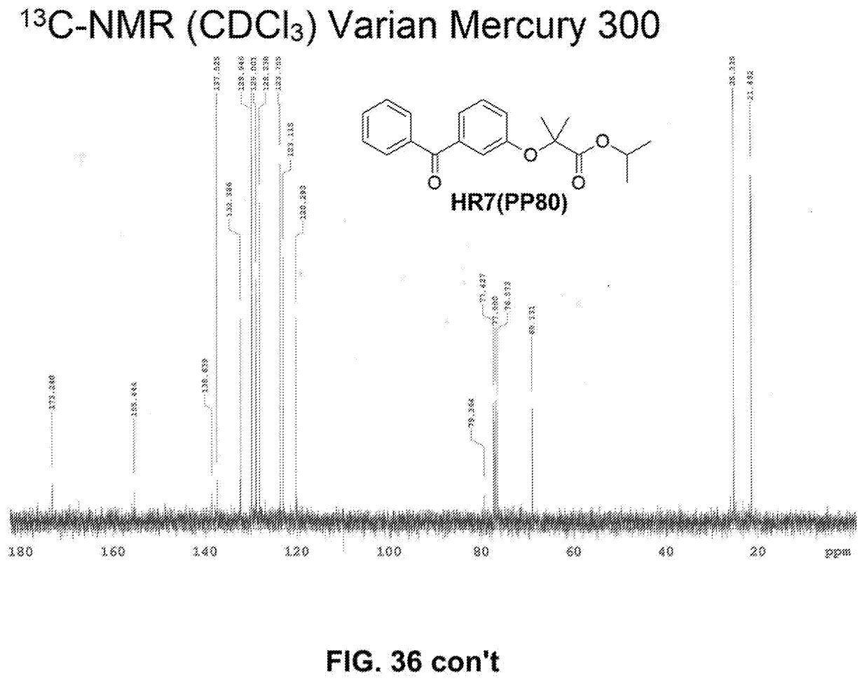

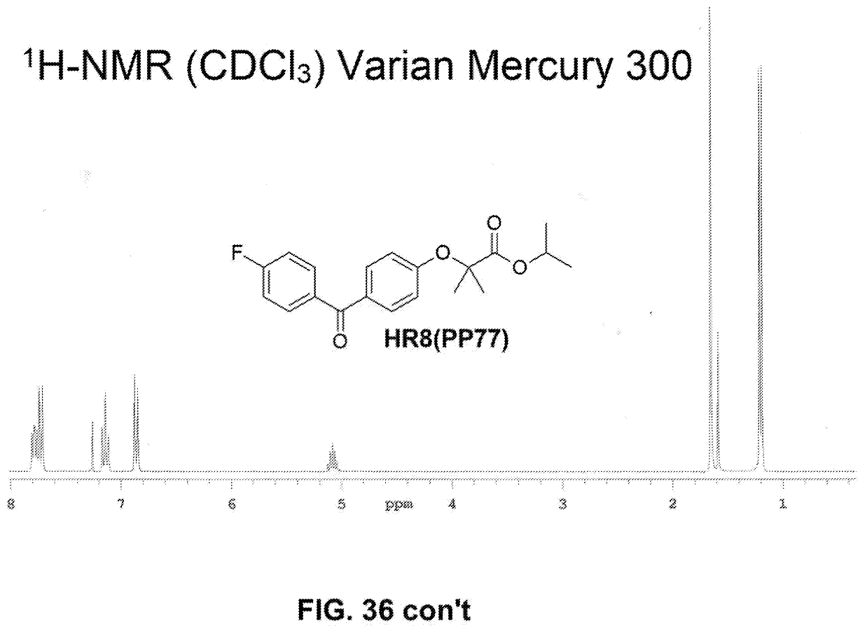

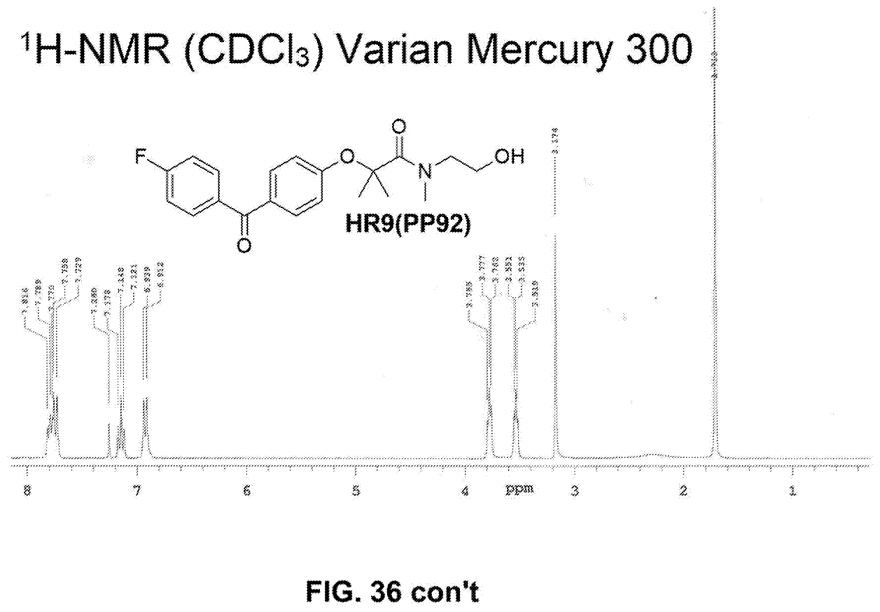

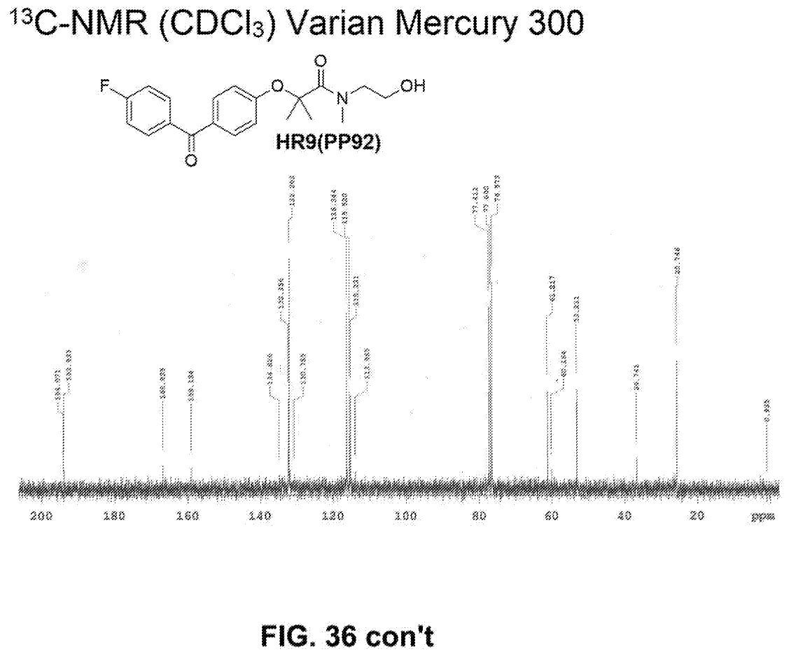

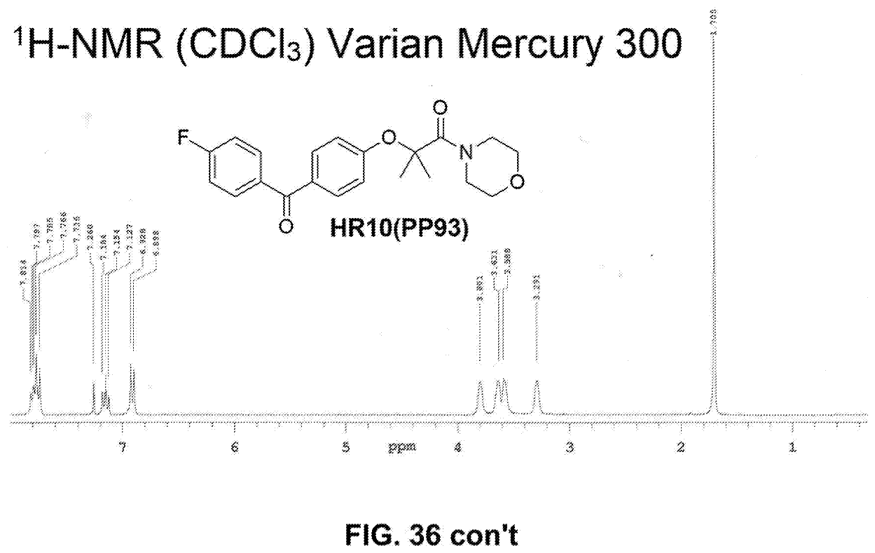

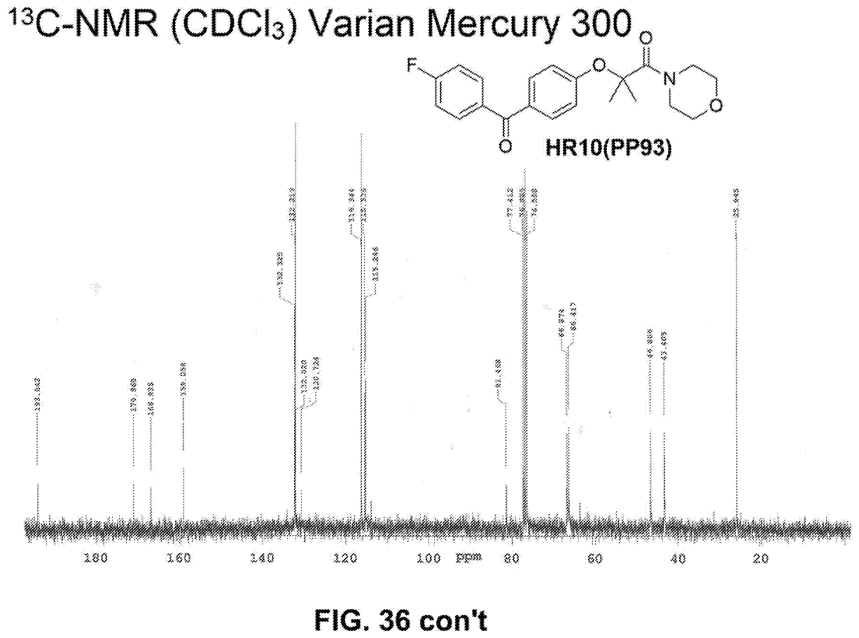

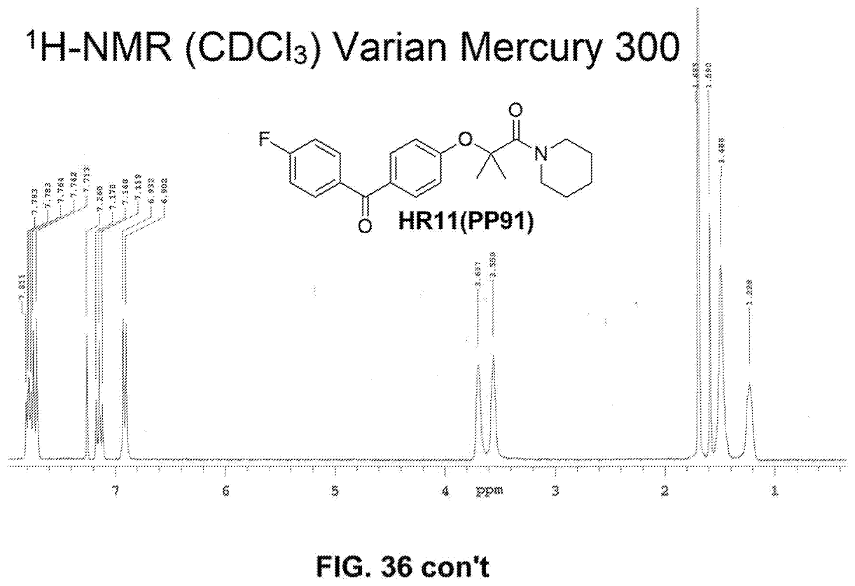

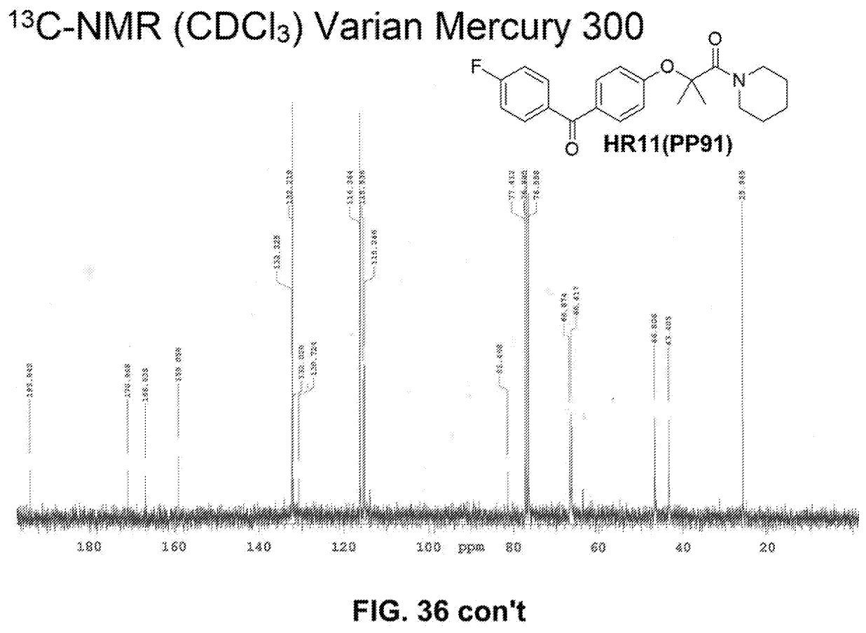

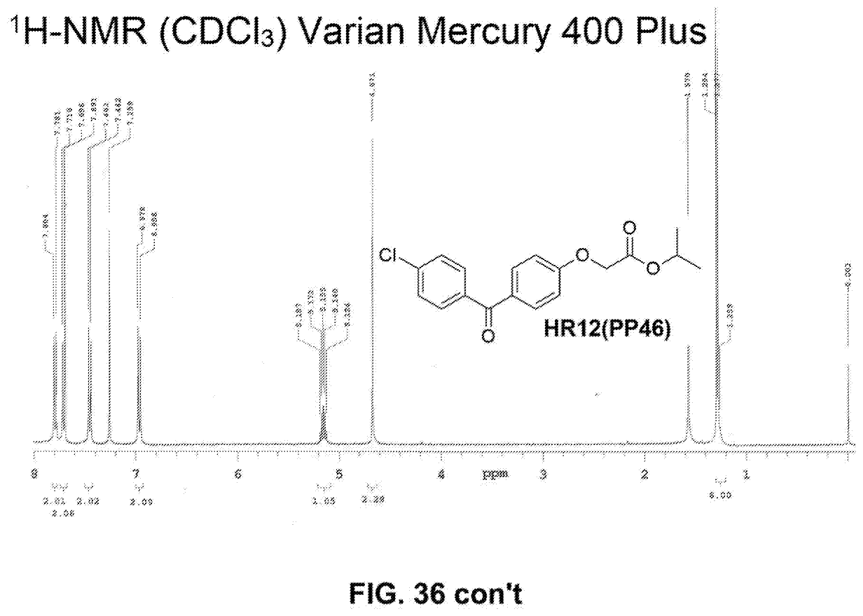

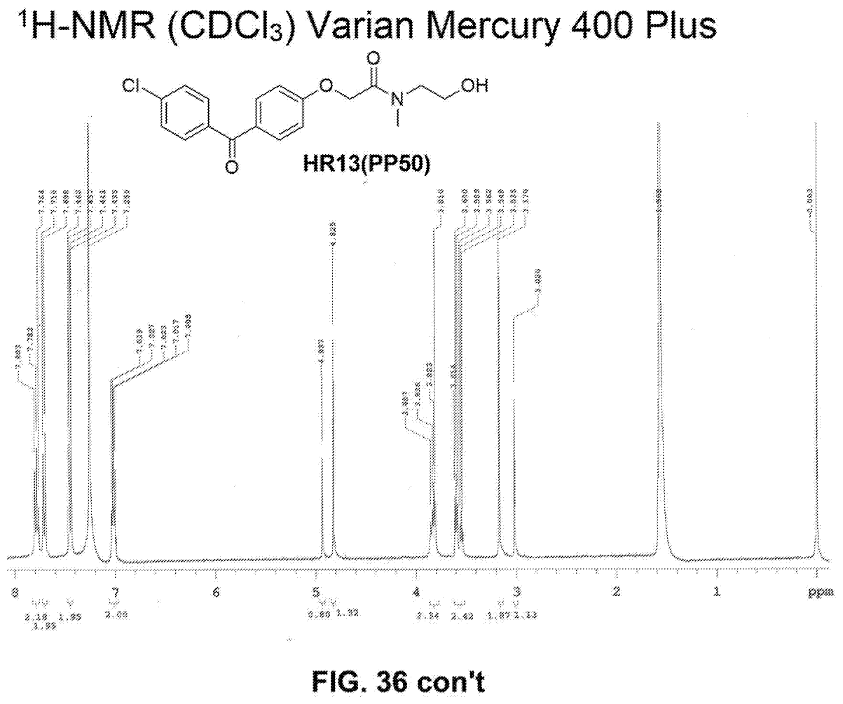

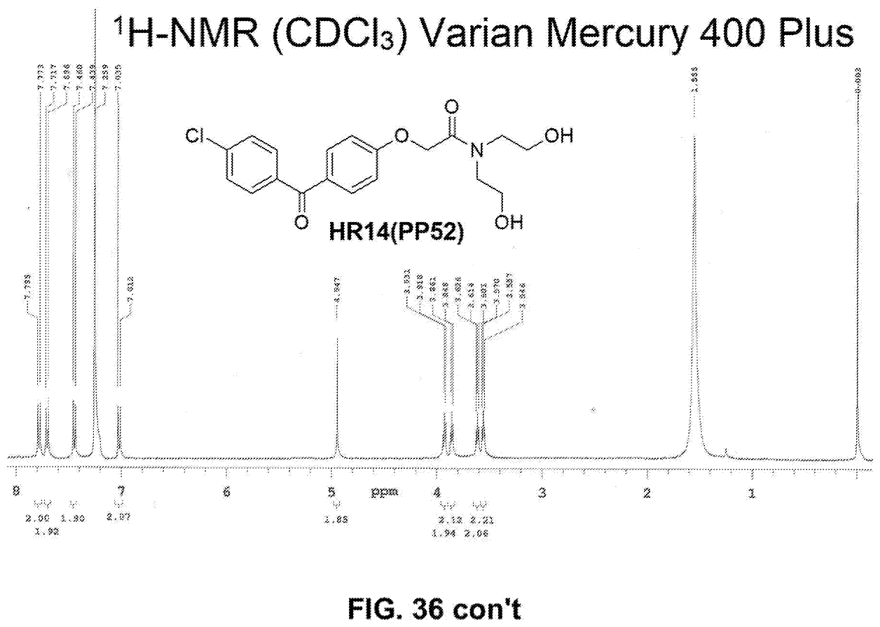

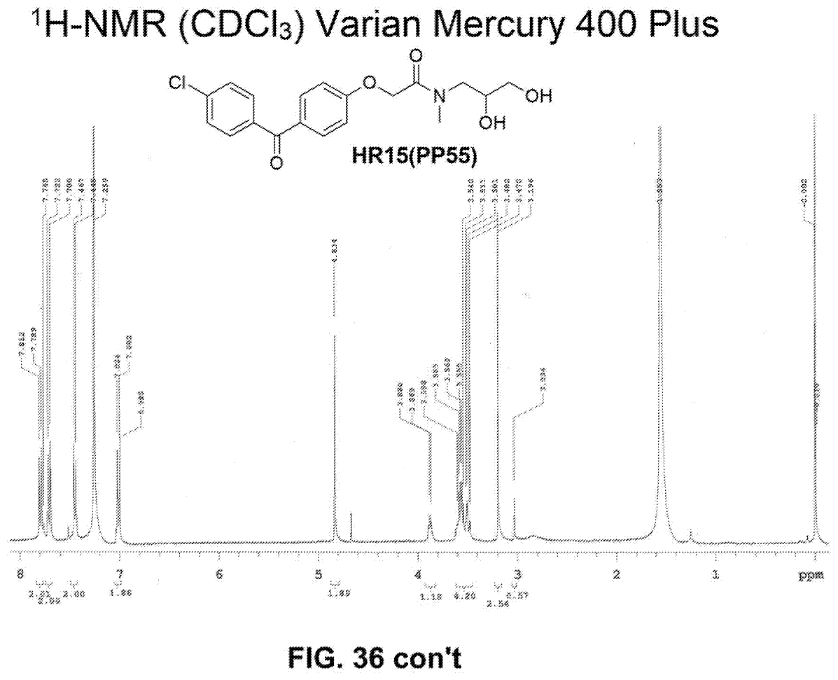

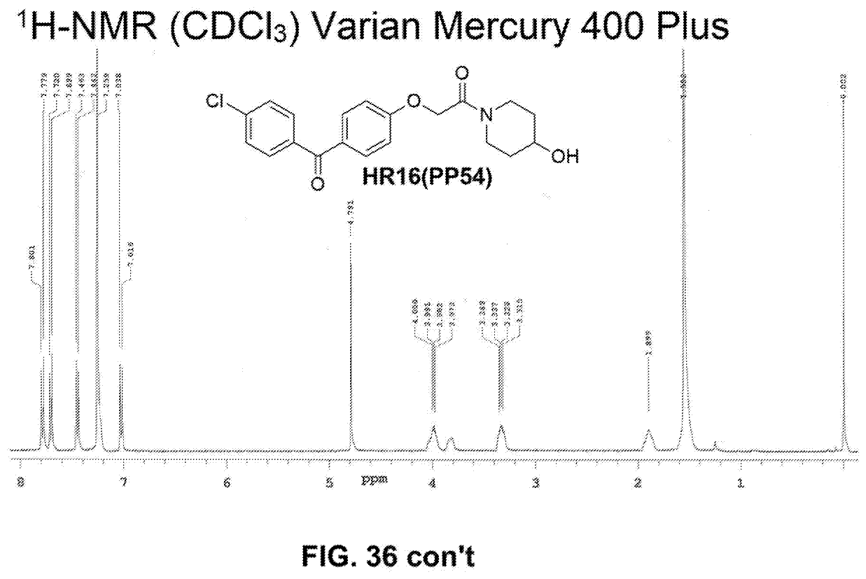

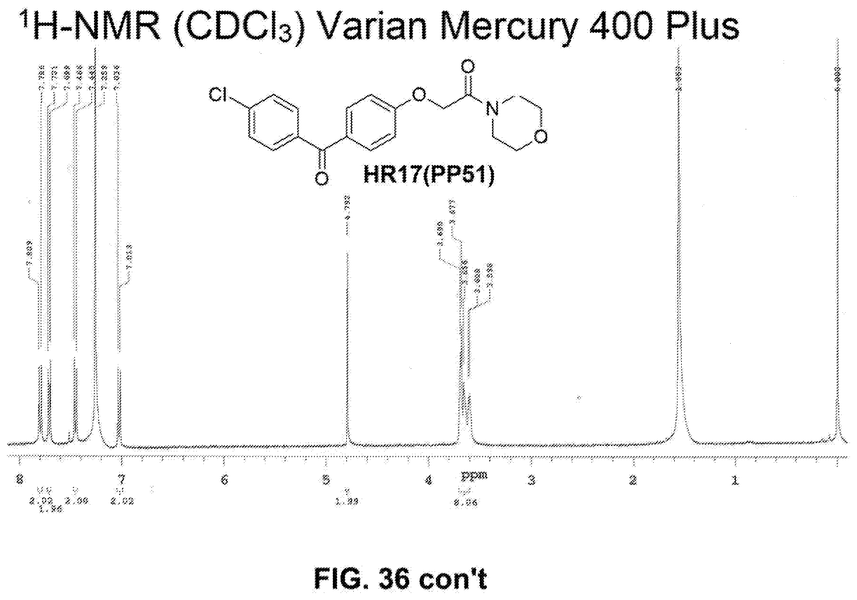

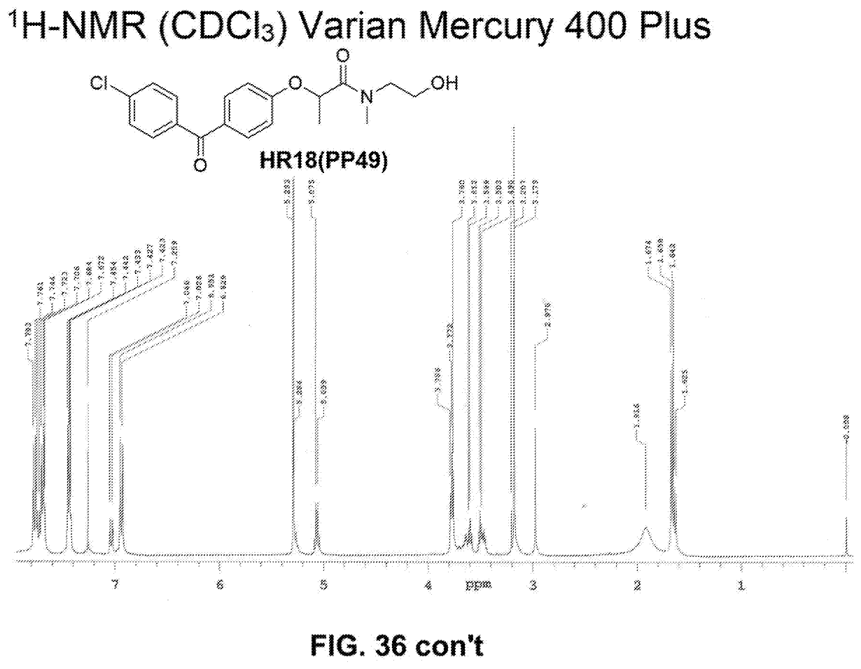

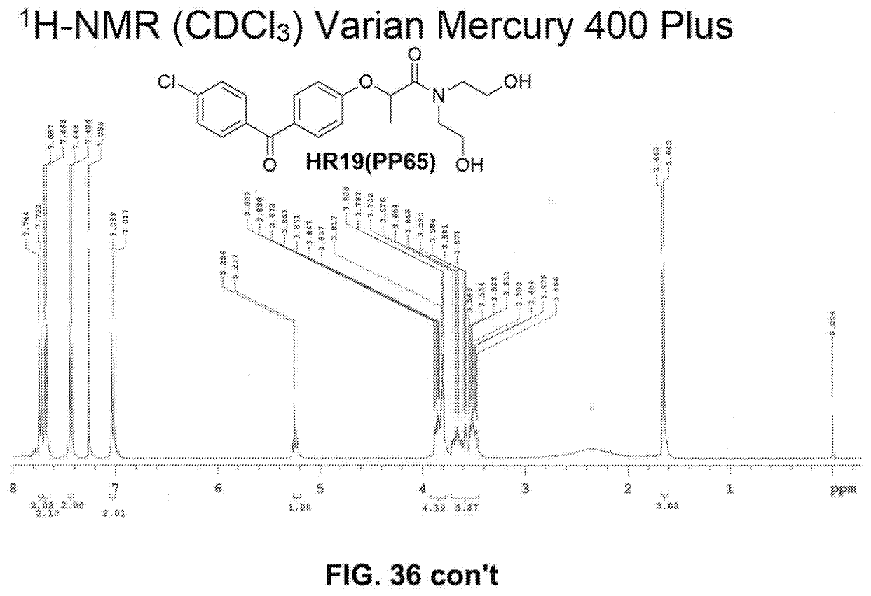

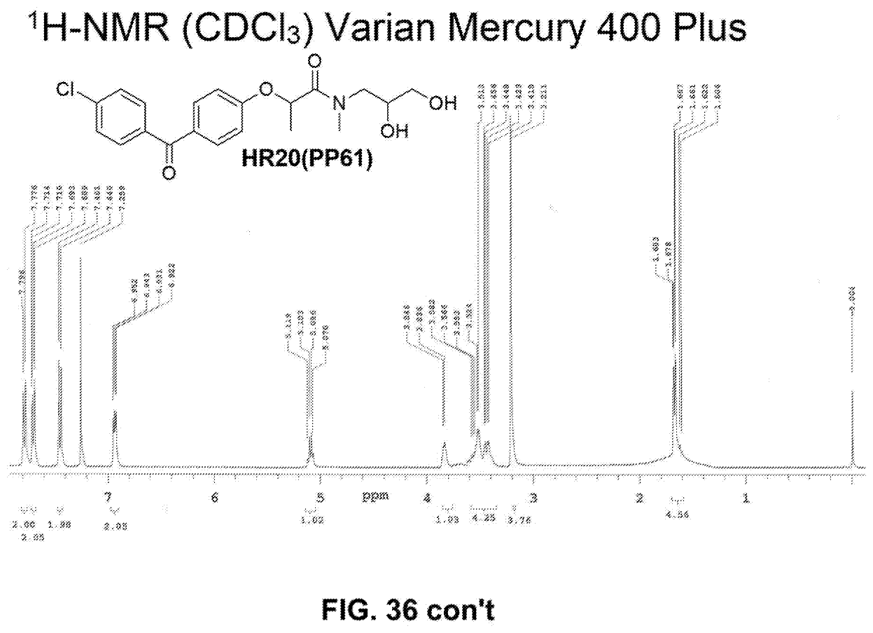

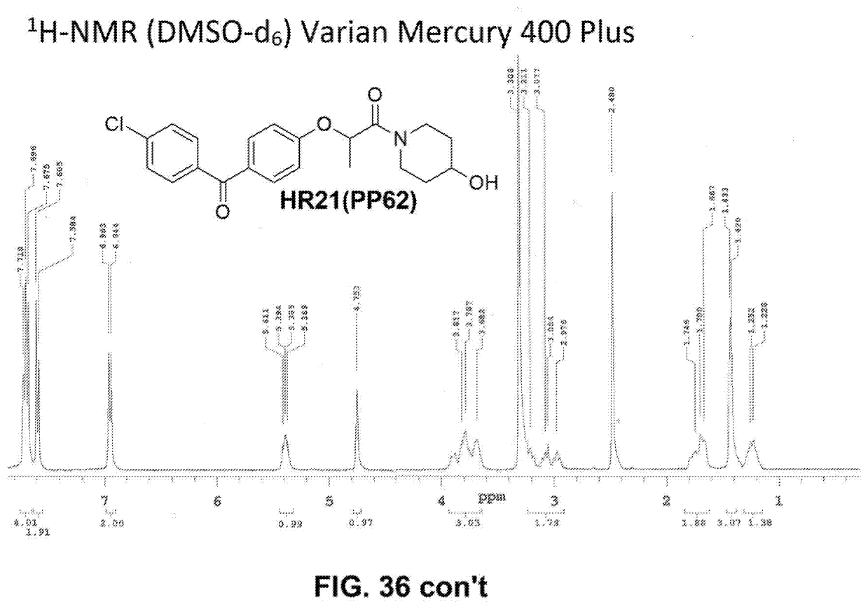

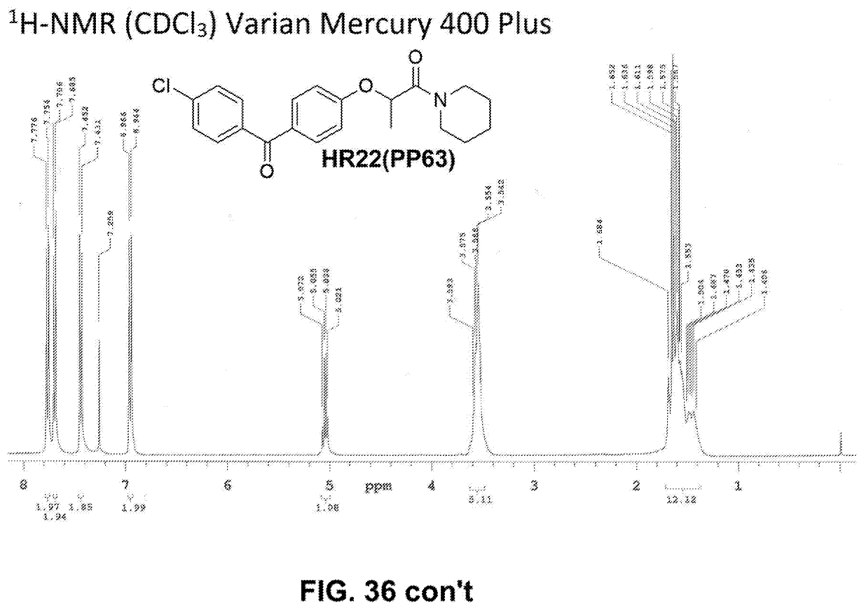

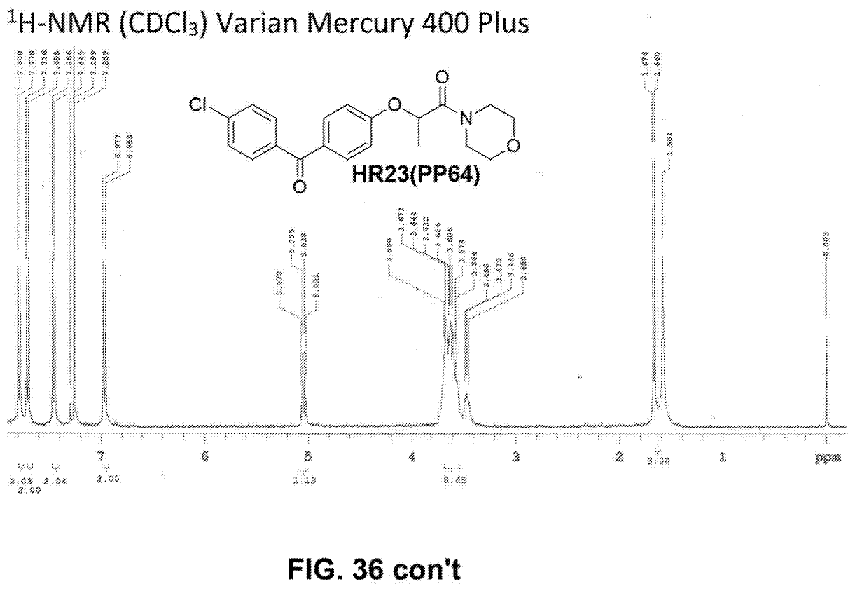

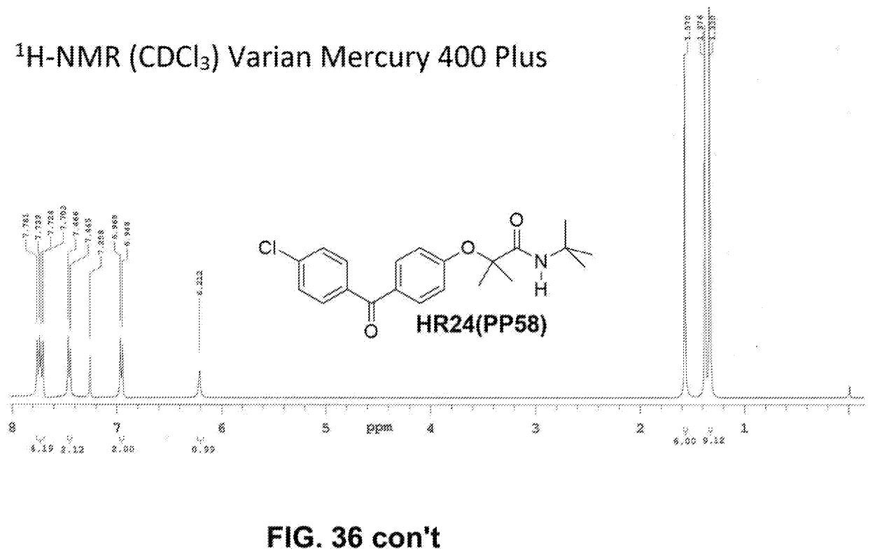

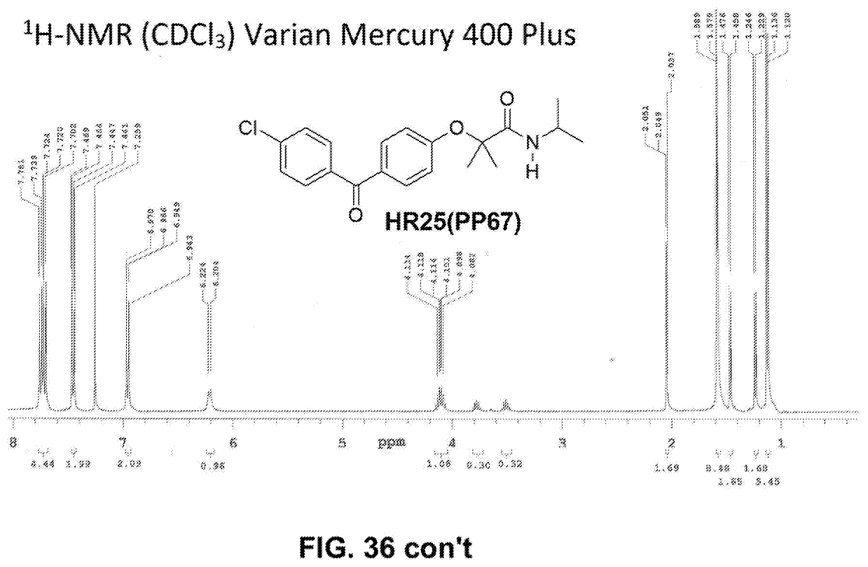

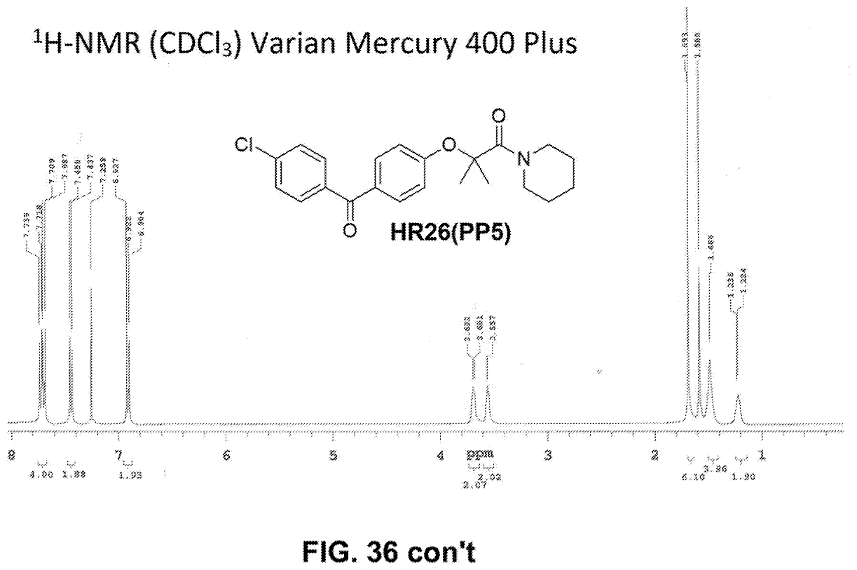

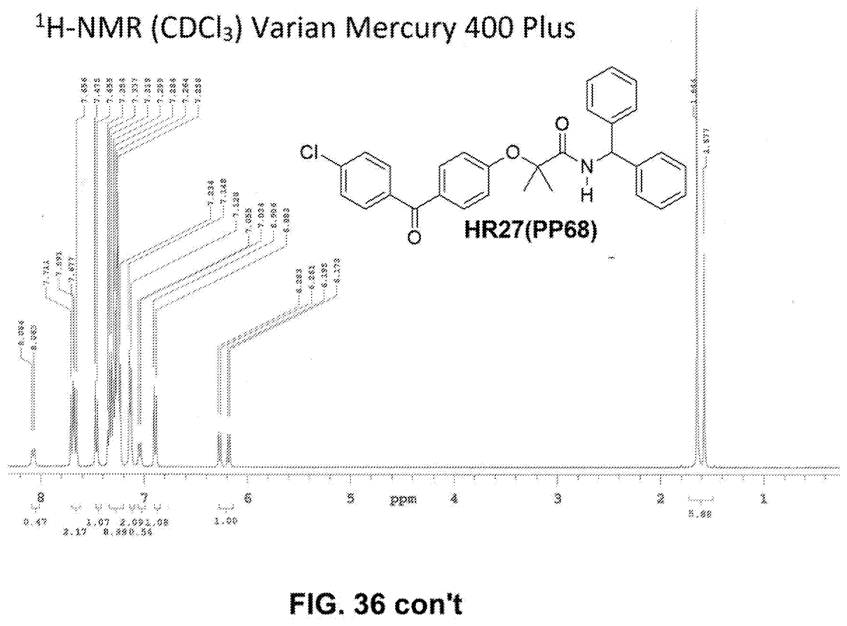

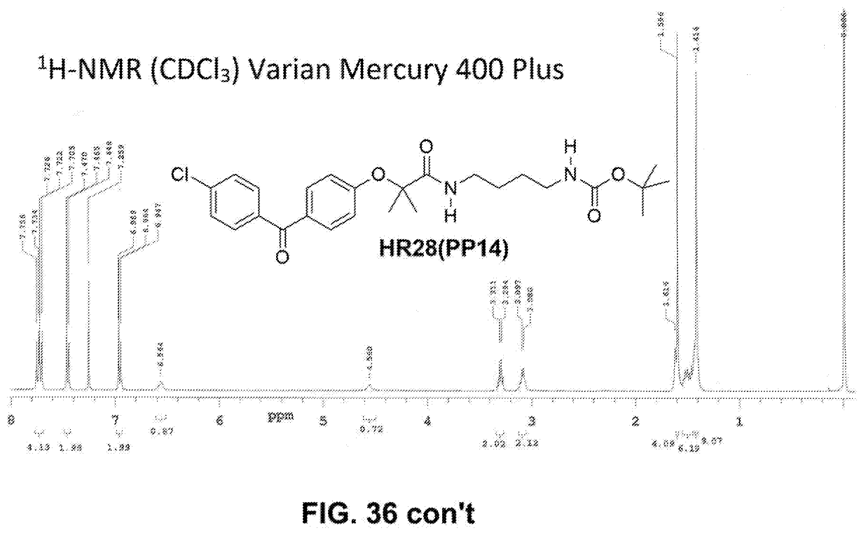

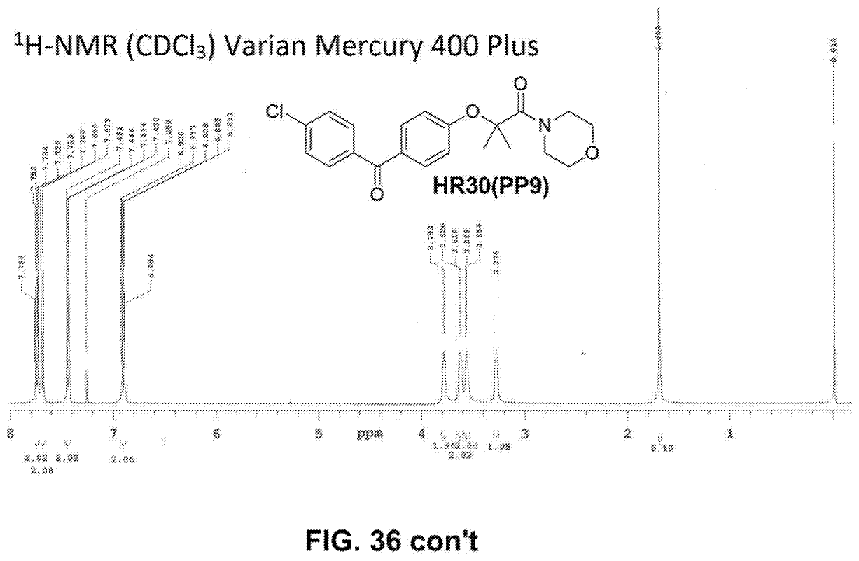

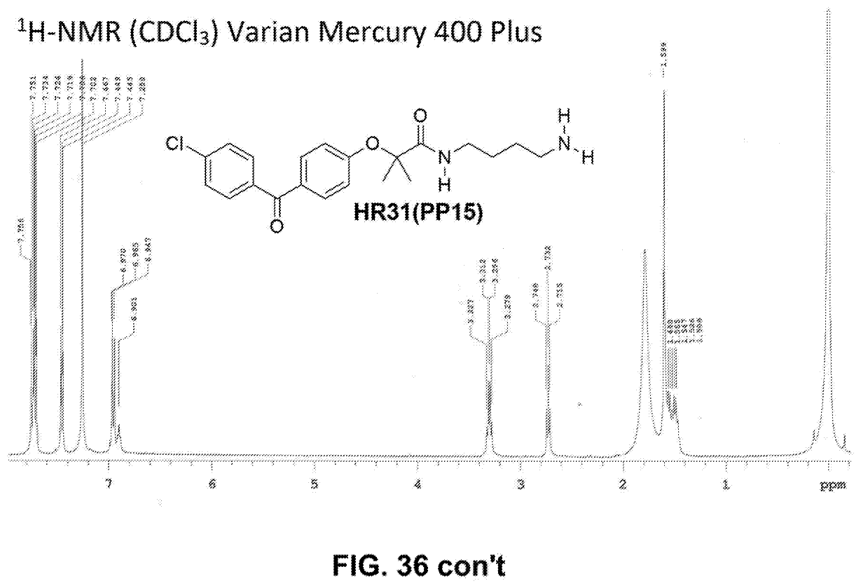

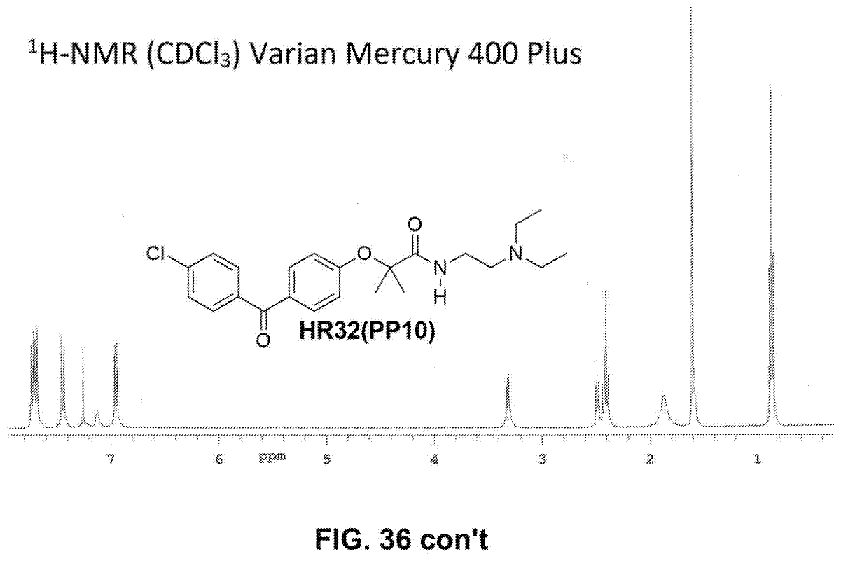

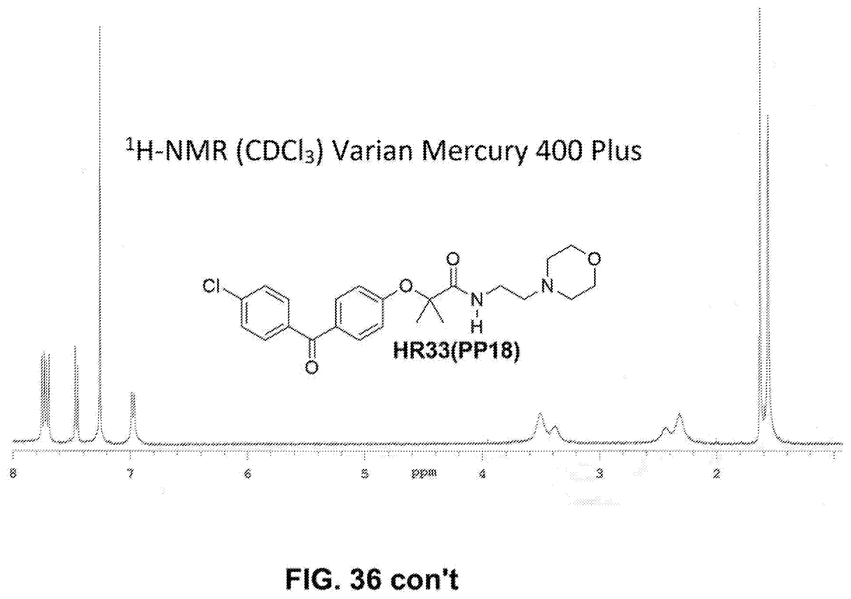

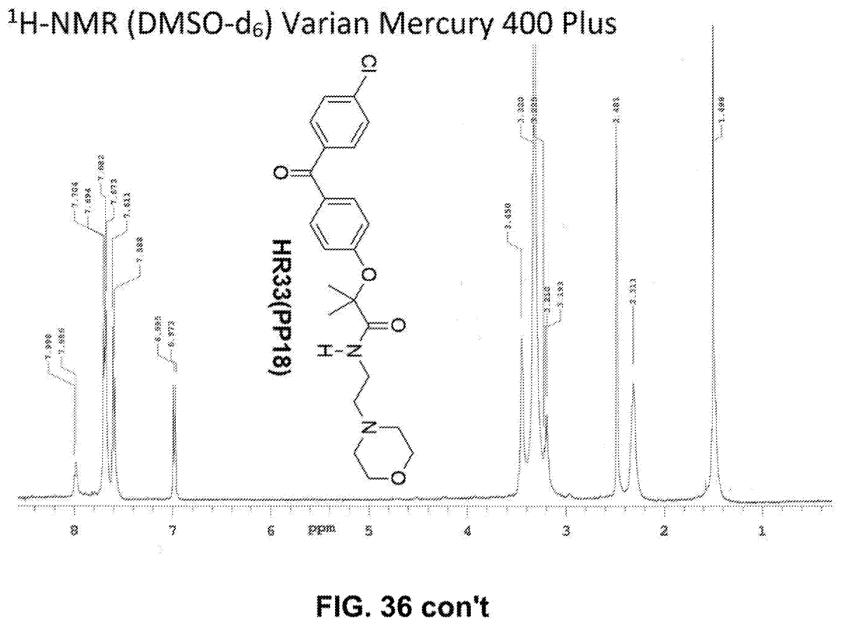

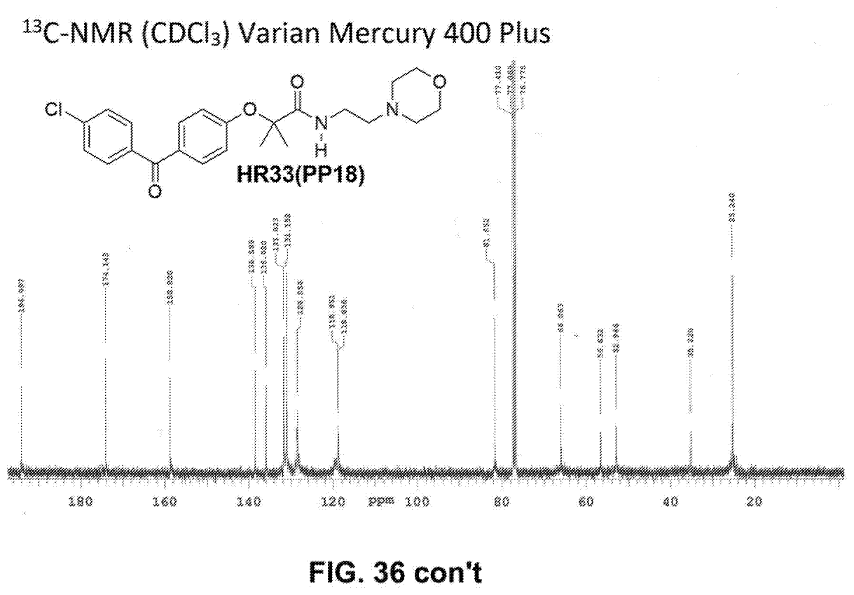

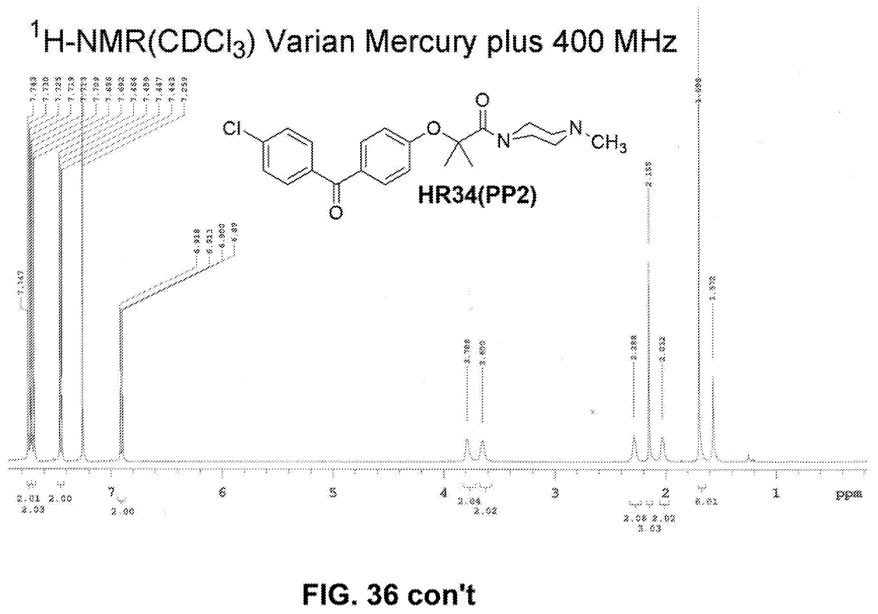

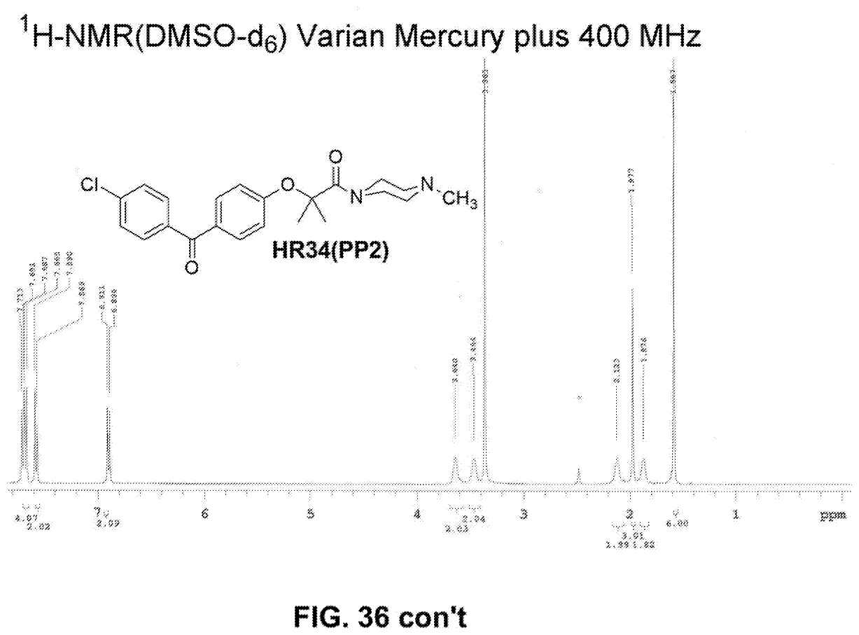

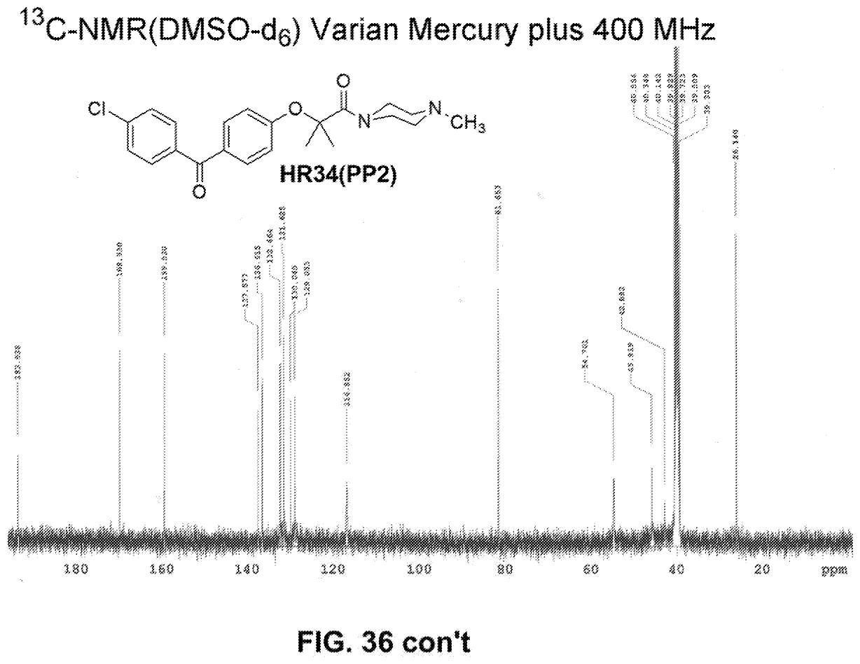

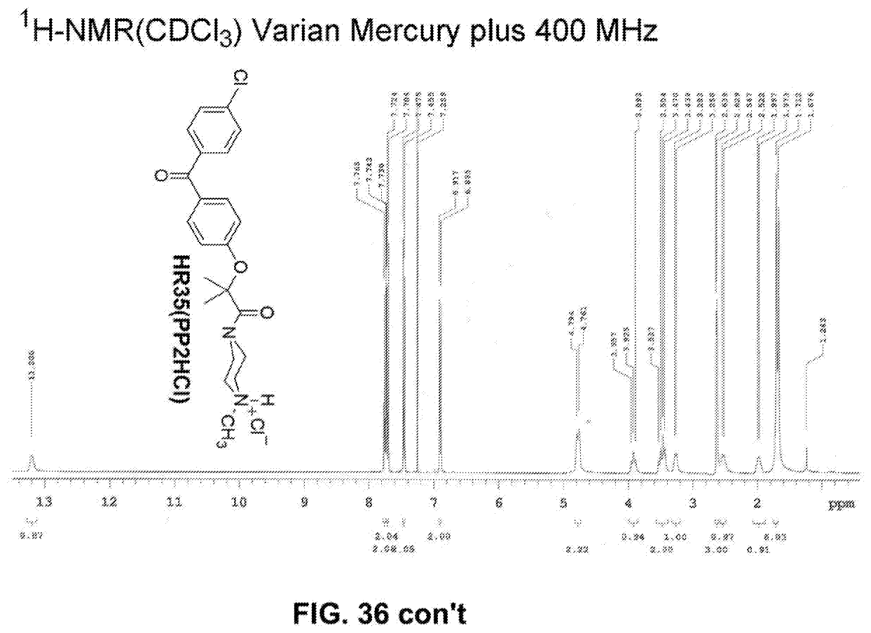

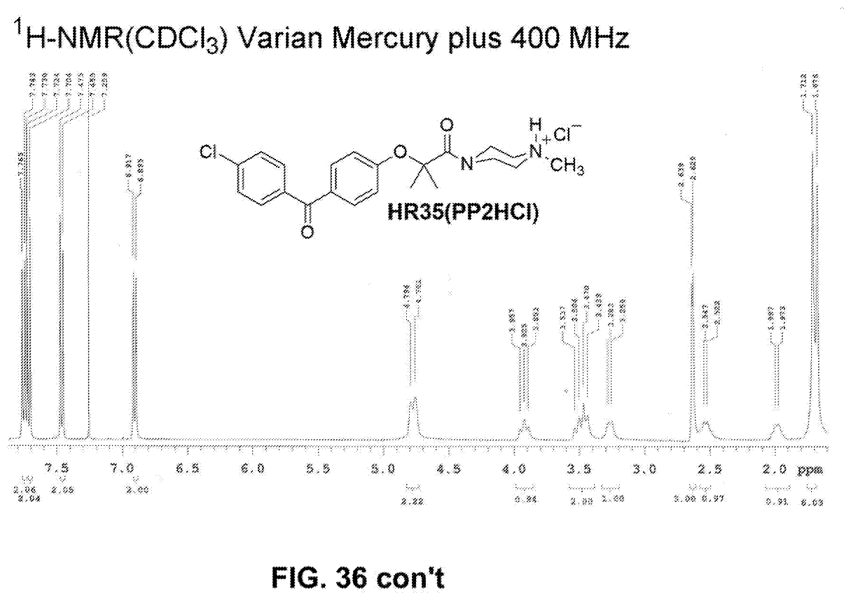

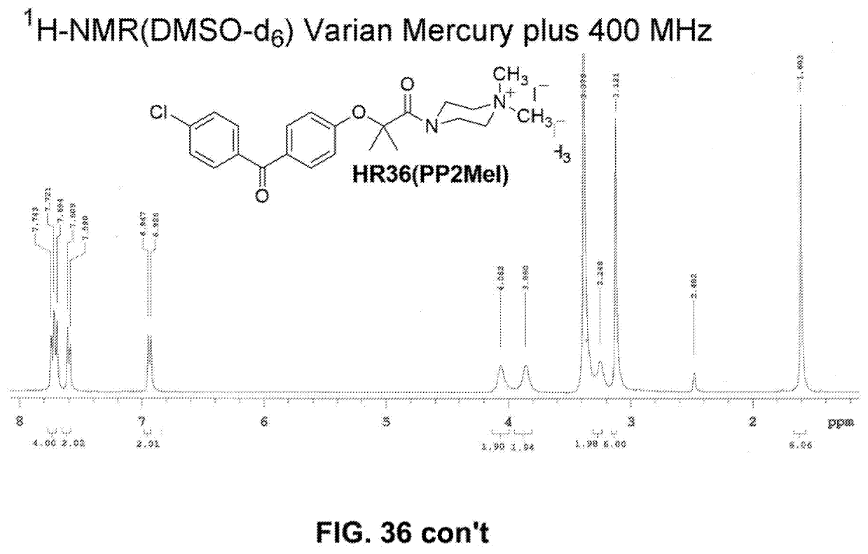

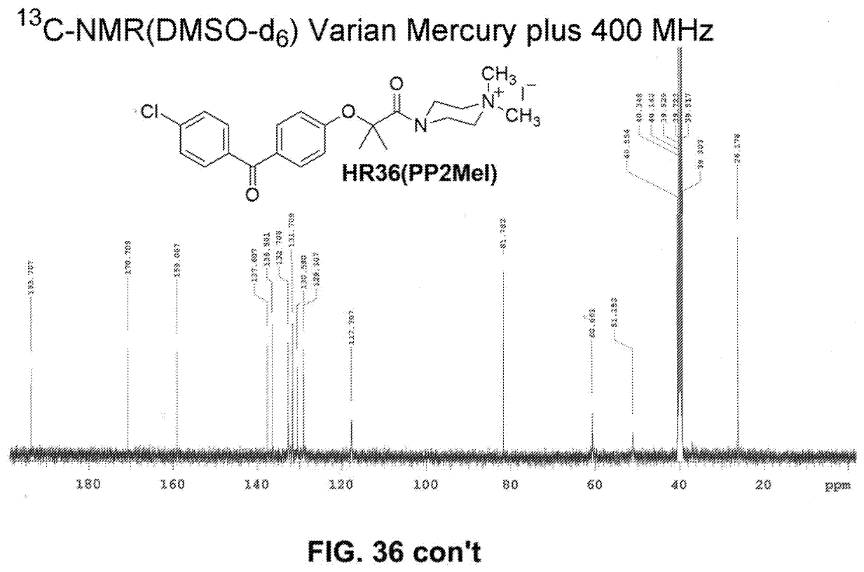

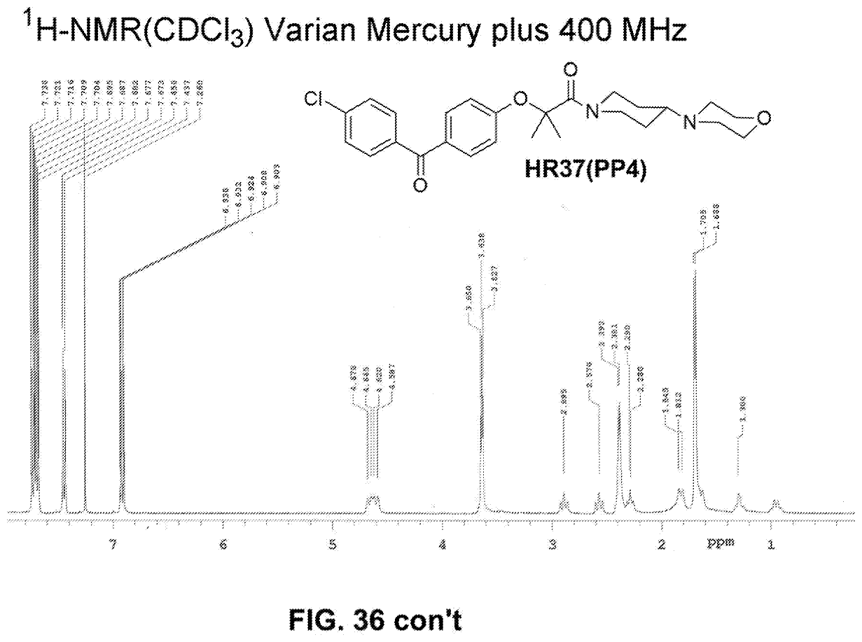

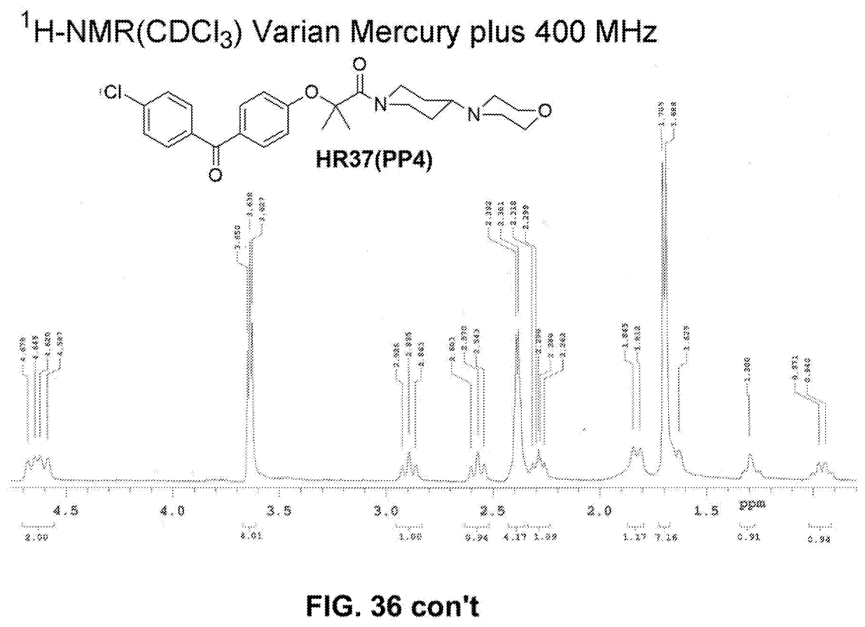

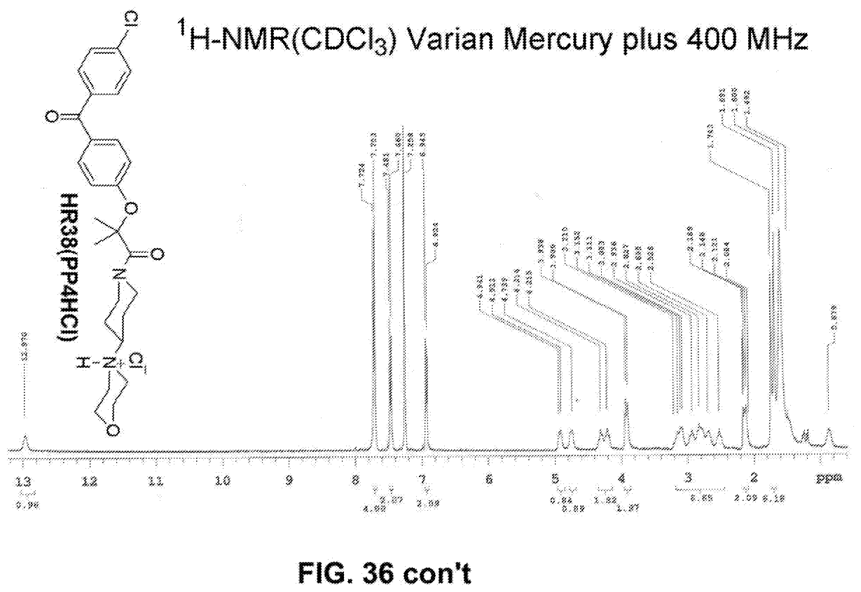

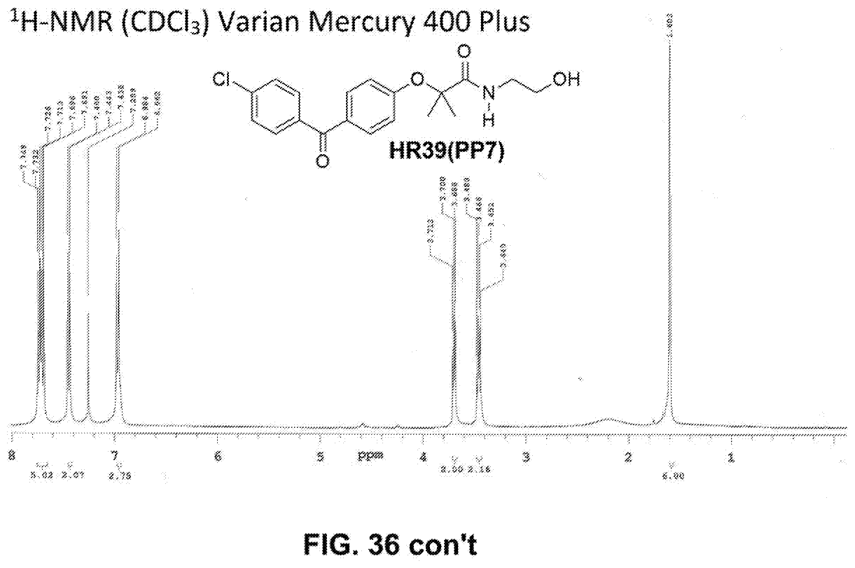

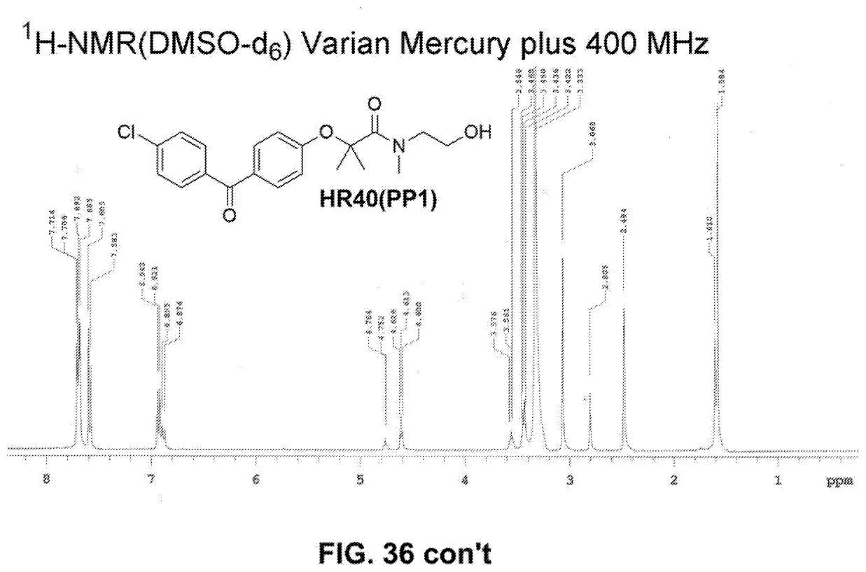

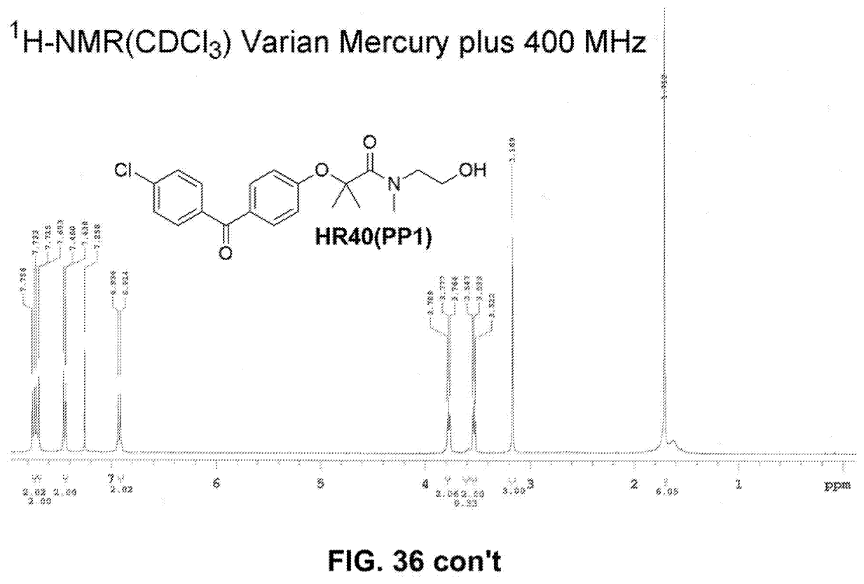

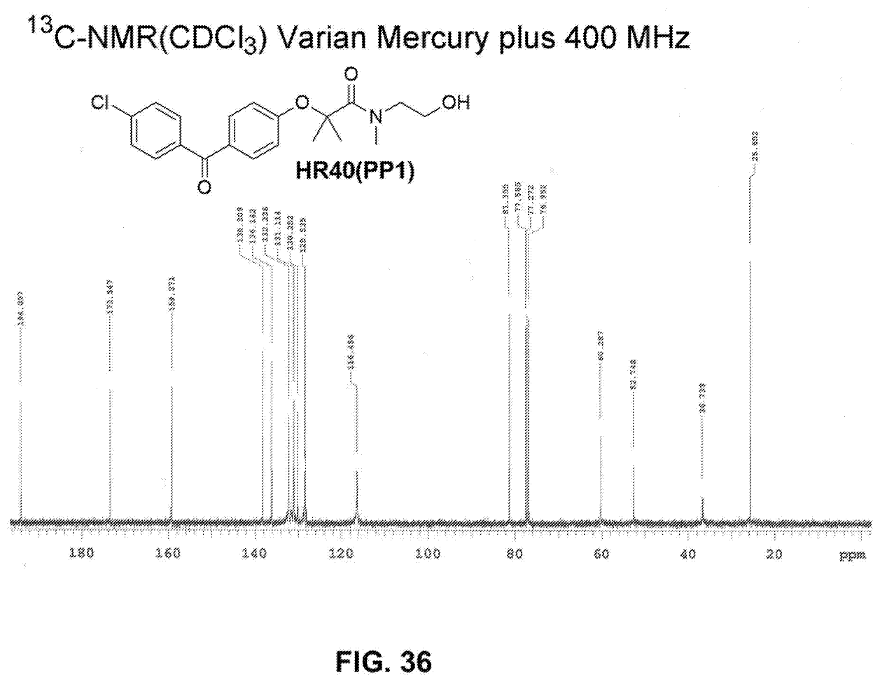

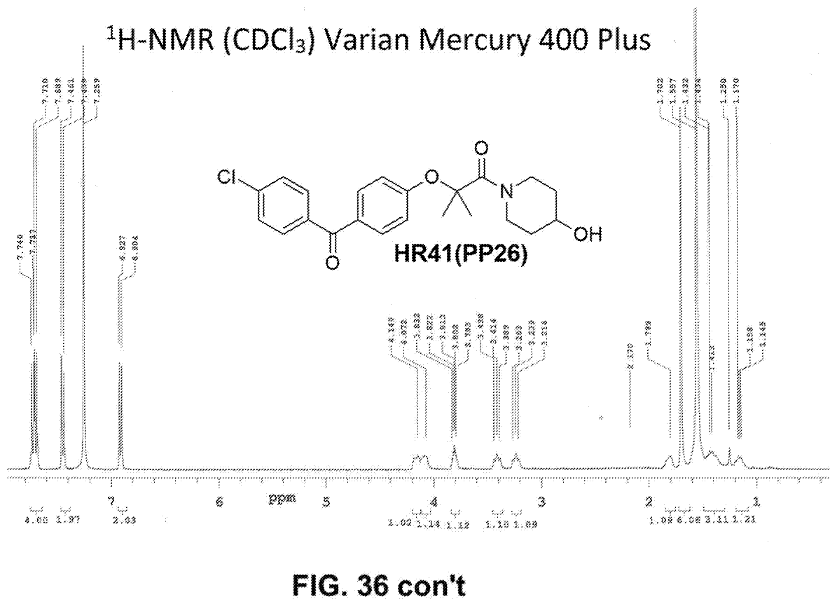

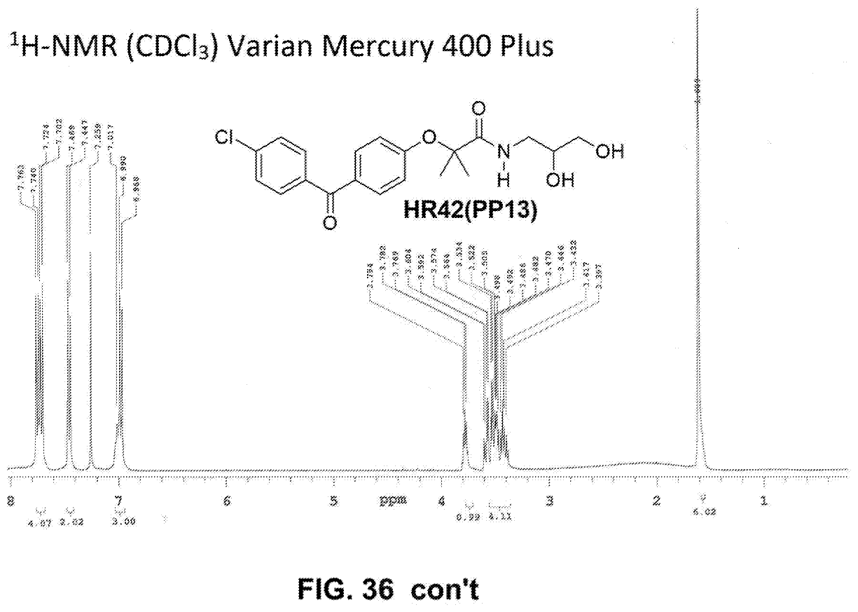

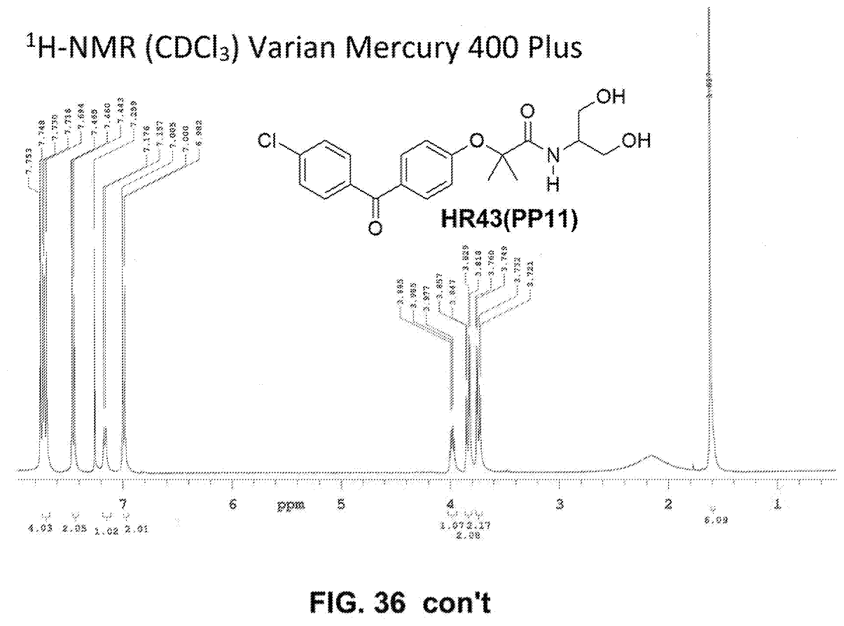

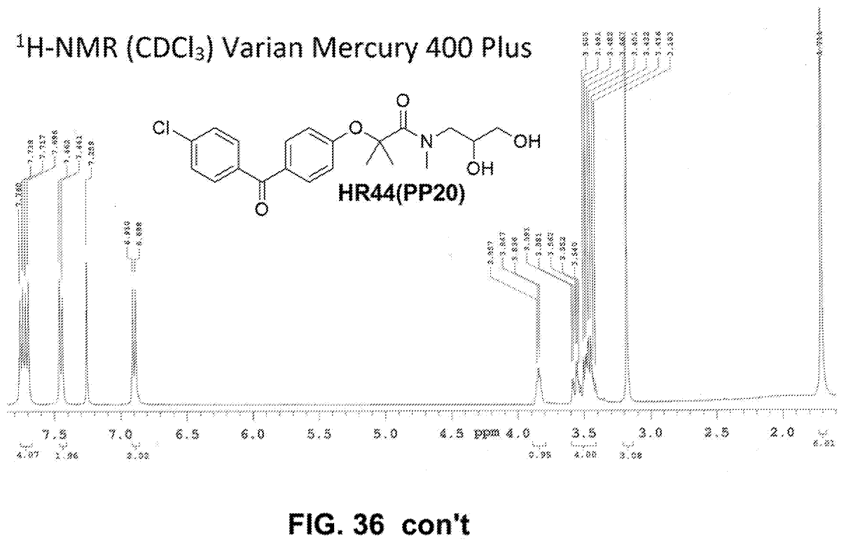

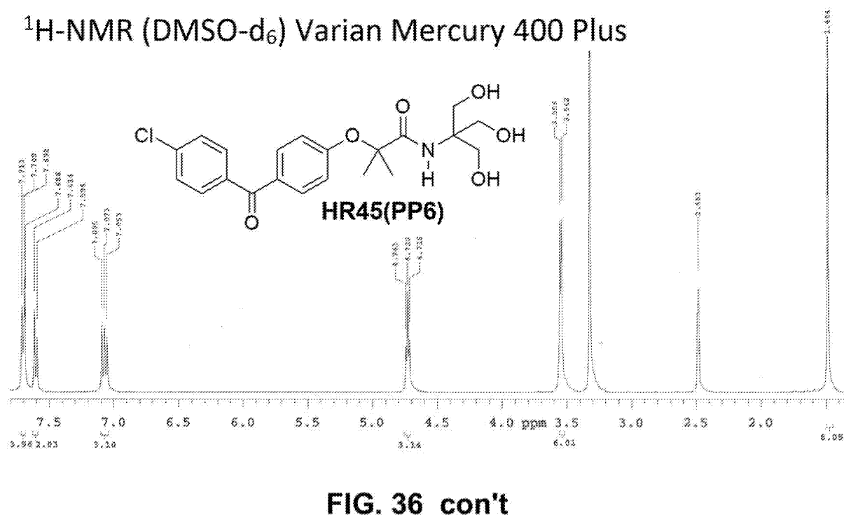

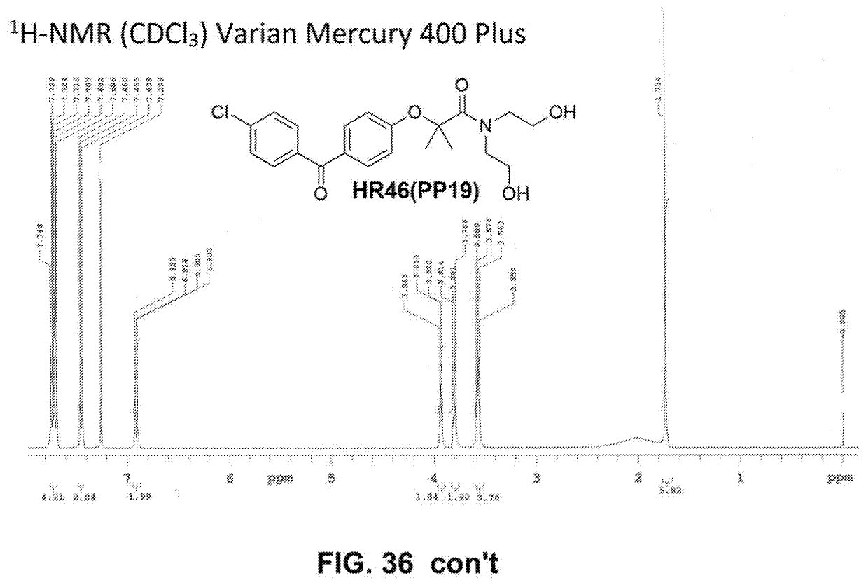

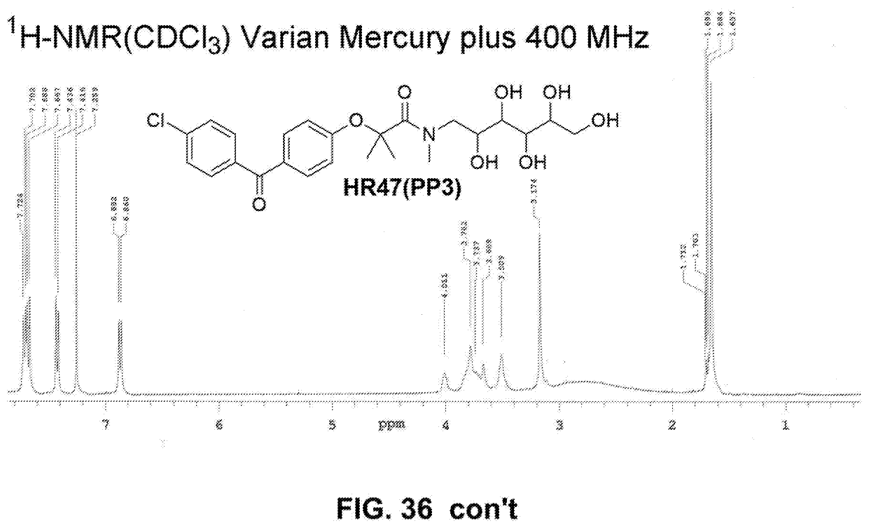

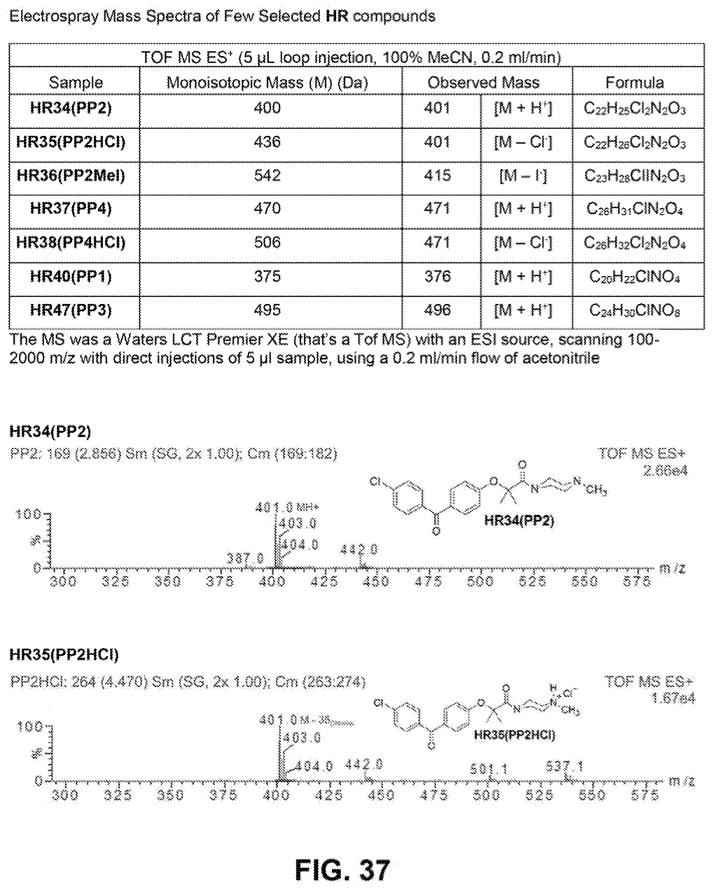

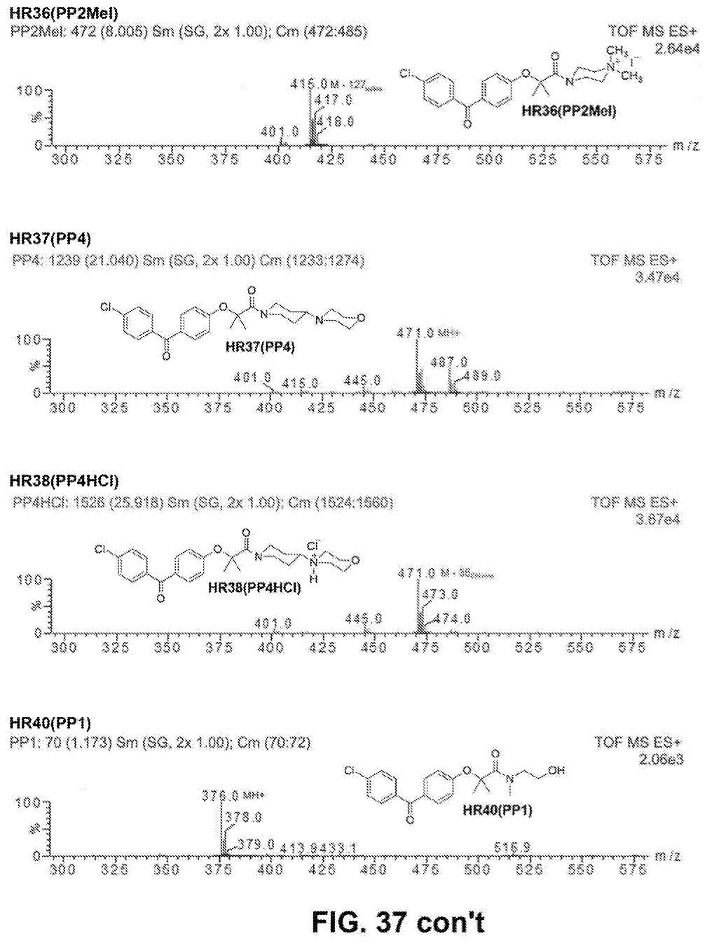

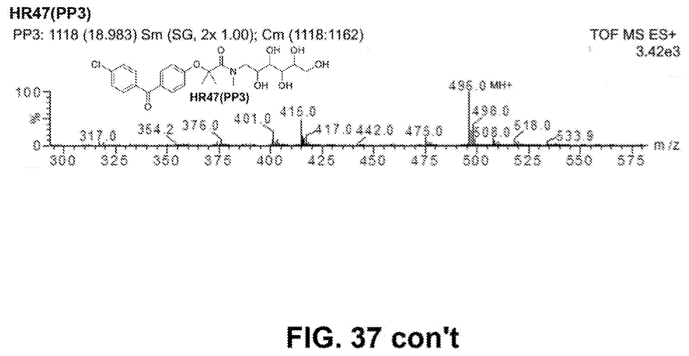

Water solubility is a major obstacle in the proper administration of drug candidates 37. One approach to increasing water solubility is to introduce hydroxy groups in the non-essential structural area of the compound. The amide moiety of new compounds listed in FIG. 33 seems to be an appropriate location to place one to several hydroxy groups. This modification of PP1 starts by replacing the N-methyl group with N—H to make HR39. Although CNS-MPO values are virtually identical for PP1 and HR39, the latter has less desirable C log P and Log BB values, indicating potentially lower cell penetration. The resulting cell viability agrees with these estimates because HR39 is significantly less potent than PP1 (FIG. 33). This finding indicates that tertiary amides are required for high anti-glioblastoma activity.

Introducing rigidity to stabilize a desired drug conformation could result in increased drug potency 38 in many instances. One of the ways to introduce molecular rigidity is by replacing a linear carbon skeleton with a cyclic carbon skeleton. This modification to PP1 (FIG. 33) resulted in the new drug candidate HR41, which is less potent than PP1.

Four drug candidates containing two hydroxy groups (HR42, HR43, HR44 and HR46) are presented in FIG. 33). Two of them (HR42 and HR43) are structural isomers, but both are secondary amides, as is HR39. Interestingly, HR42 and HR43 activities (CV) are almost identical, indicating that the presence of the tertiary amide moiety is more important than the presence of the two hydroxy groups. Two tertiary amides with two hydroxy groups, HR44 and HR46, are also structural isomers. They are both more potent than the secondary amide HR39, but the tertiary amide HR46, with two CH₂CH₂OH moieties, is almost as potent as PP1. However, introducing three (HR45) or more (HR57) hydroxy groups slightly decreases the potency, possibly because the compounds are becoming substantially more hydrophilic, as is demonstrated with lower Clog, MPO, and log BB estimated values (FIG. 33). In conclusion, from this group (HR39-HR47) the best candidate is HR46 due to its low CV and acceptable CNS-MPO values that are very similar to the prototype drug, HR40 (PP1).

In conclusion, we have identified four drug candidates, similar to PP1 (HR40), that have strong in vitro anti-glioblastoma activity but have physical properties that may contribute to the improved brain tumor penetration (FIG. 34). The IC₅₀ concentration of these compounds is around 10 μM, which is an acceptable therapeutic concentration for most clinically relevant anticancer drugs 39. By exploring the computed structural and functional properties of benzylphenoxyacetamide (BPA), along with cell viability, it was demonstrated that two methyl groups in the 2-position of BPA are important for retaining anti-glioblastoma activity. Substitution of the chloro-substituent in the 4-position of the benzophenone moiety, resulted in a significant loss of anticancer activity of the modified compound. The molecular rigidity between the two aromatic rings of the benzophenone moiety is essential because both methylene and oxygen replacement of the carbonyl group resulted in lost or diminished anti-tumoral activity. It was demonstrated that tertiary amides are more potent than secondary amides, which, in turn, are more potent than primary amides. In tertiary amides, it is important to keep one substituent small (methyl), while the other group can be a hydroxy or nitrogen substituted alkyl. In addition to the evaluated chemical changes in the BPA structure, there is still a lot of flexibility for additional structural variations mostly on the amide moiety (FIG. 18; Region D). Exploring these additional structural variations and applying experimental and computational analyses of BBB penetration in combination with drug efficacy testing using syngeneic and patient-derived glioblastoma animal models might produce even more suitable anti-glioblastoma drug candidates.

Experimental Strategies.

All starting materials were reagent grade and purchased from Sigma-Aldrich, ArkPharm, TCI America, and AbaChemScene. ¹H-NMR spectra were recorded on Varian Mercury 300 and Varian Mercury 400 Plus instruments in CDCl₃, DMSO-d₆, using the solvent chemical shifts as an internal standard. Electrospray Mass Spectroscopy (EMS) was recorded on Waters LCT Premier XE (that's a Tof MS) with an ESI source, scanning 100-2000 m/z with direct injections of 5 μl sample, using a 0.2 ml/min flow of acetonitrile. All molecular physical properties were calculated using Marvin Sketch software. All computed molecular descriptors were generated by ChemAxon MarvinSketch version 19.4. Electrostatic potential maps were calculated with PM3 semi-empirical method as implemented in Spartan '18 v 1.1.0.

Cell culture and viability assays. Human glioblastoma LN-229 cells were maintained as semi-confluent monolayer culture in DMEM with 1 g/L glucose, sodium pyruvate, L-glutamine (Corning) supplemented with 10% heat inactivated FBS (Gibco) and P/S (50 units/mL of penicillin and 50 μg/mL of streptomycin) at 37° C. in a 5% CO2 atmosphere. Prior to treatment, cells were plated well in 96-well plates (BD Falcon) at initial density of 2×10³ cells/cm². Stock solutions of the compounds were prepared in DMSO, diluted in cell culture medium and added to the cells in triplicate for every experimental condition 24 h after plating (final concentration 25 μM). For the vehicle control, DMSO was used at 0.5%. MTT assay⁴⁰ (measuring cell metabolic activity) was performed after a 72 h incubation in the presence of the compounds as previously described. Following 1 h incubation with MTT, formazan crystals were dissolved in 5 mM HCl in isopropanol and absorbance read at 540 nm. Data represent mean values expressed as percentage of vehicle control±SD. Phase contrast images of treated cells were taken 48 hours following the treatment with a BZ-X800 fluorescence microscope (Keyence) equipped with a 20× objective.

Method A. Preparation of isopropyl 2-(4-(4-chlorobenzoyl)phenoxy)acetate (3n). Water (10 ml) solution of sodium hydroxide (410 mg; 10.25 mmol), (4-chlorophenyl)(4-hydroxyphenyl)methanone (2.3 g; 0.1 mol) and benzene (100 ml) was refluxed for 10 minutes and water was azeotropically removed by using Deen-Stark distillation apparatus followed by removal of benzene under reduced pressure. White powdery sodium phenoxide was mixed with dry isopropanol (100 ml) and isopropyl bromoacetate (1.9 g; 10.5 mmol). Resulting mixture was stirred with sonication for 1 hour and refluxed for 4 hours. Solvent was evaporated to solid residue and mixed with dichloromethane (100 ml) and water (100 ml). Water layer was discarded, and organic layer was washed with 5% sodium carbonate (3×50 ml) and dried over anhydrous sodium carbonate. After solvent evaporation white residue was dried under vacuum to give white solid product in 93% (3.1 g) isolated yield. ¹H-NMR (400, DMSO-d6) δ 7.79 (2H, d, J=9.2 Hz), 7.71 (2H, d, J=8.8 Hz), 6.97 (2H, d, J=8.8 Hz), 5.16 (1H, septet, J=7.2 Hz), 4.67 (2H, s), and 1.28 (6H, d, J=7.2 Hz) ppm.

Method B. Isopropyl 2-(3-benzoylphenoxy)-2-methylpropanoate (4f). Isopropanol (300 ml) suspension of sodium carbonate (21.2 g; 200 mmol), (3-hydroxyphenyl)(phenyl)methanone (4 g; 20 mmol) and isopropyl 2-bromo-2-isobutirate (4.2 g; 20 mmol) was refluxed with stirring for 3 days. After cooling to room temperature white solid was separated by filtration washed with isopropanol (3×20 ml). Combined filtrates were evaporated to an oily residue. This residue was mixed with water/chloroform (100 ml/200 ml). Water layer was discarded, and chloroform layers was washed with 5% sodium hydroxide (3×50 ml), water (3×50 ml), and dried over anhydrous sodium carbonate. After filtration chloroform was evaporated to oily residue that standing at 5° C. overnight give white solid in 92% (6 g) yield. ¹H-NMR (300 MHz, CDCl₃) δ 7.77 (2H, d, J=6.9 Hz), 7.68 (1H, t, J=6.9 Hz), 7.48 (2H, d, J=7.5 Hz), 7.5-7.3 (3H, m), 7.27 (1H, s), 7.07 (1H, d, J=6.9 Hz), 5.06 (1H, septet, J=6.3 Hz), 1.60 (6H, s), and 1.18 (6H, d, J=6.9 Hz). ¹³C-NMR (200 MHz, CDCl₃) δ 173.2, 155.4, 128.6, 137.5, 129.9, 129.0, 128.2, 127.5, 123.8, 123.1, 120.3, 79.4, 69.1, 25.3 and 21.5 ppm

Method C. Acid preparation. Preparation of 2-methyl-2-(4-phenoxyphenoxy)propanoic acid (le). Water (10 ml) solution of sodium hydroxide (0.42 mg; 10.5 mmol), benzene (100 ml) and 4-phenoxyphenol (1.86 g; 10 mmol) was stirred at room temperature for one hour. Water was evaporated away by Dean-Stark distillation. Remaining benzene was removed under reduced pressure. White powdery residue was mixed with isopropanol (200 ml) and isopropyl 2-bromo-2-methylpropanoate (2.1 g; 10 ml). Resulting mixture was stirred with sonication at 60° C. for two hours followed by refluxing overnight. After cooling to room temperature 5% sodium hydroxide (100 ml) was added and resulted mixture was refluxed for 2 hours. Solvent was evaporated to solid residue mixed with water (150 ml) and acidified with concentrated hydrochloric acid to pH ˜3. Resulting white suspension was mixed with chloroform (100 ml) and chloroform layer was separated, washed with water (3×50 ml) and then with 10% sodium carbonate (100 ml). Sodium carbonate layer was acidified with concentrated hydrochloric acid to pH˜ 3. Formed white precipitate was separated by filtration, washed with water and dried at room temperature under vacuum to give pure product in 92% yield (2.5 g). ¹H-NMR (400 MHz, CDCl₃) δ 7.31 (2H, t, J=7.6 Hz), 7.08 (1H, t, J=7.6 Hz), 6.96 (2H, d, J=7.6 Hz), 6.93 (4H, s), and 1.58 (6H, s) ppm.

Method D (nitrogen unsubstituted amides). Preparation of 2-(4-(4-chlorobenzoyl)phenoxy)-2-methylpropanamide (AA). Dichloromethane (10 ml) suspension of fenofibric acid (FFA, 318 mg; 1 mmol) and two drops of DMF was stirred at room temperature for 3 hours. Clear dichloromethane solution was evaporated at 30° C. under reduced pressure. Remaining white solid residue was dissolved in tetrahydrofuran (20 ml) and mixed with aqueous ammonia (10 ml; 3.08 g; 0.17 mol) and stirred at room temperature for one hour. Resulting mixture was mixed with dichloromethane (50 ml) and water (50 ml). Organic solvent was separated, washed with water (3×50 ml), 5% sodium carbonate (50 ml), and dried over anhydrous sodium carbonate. Solvent was evaporated to give white solid product that was recrystallized from dichloromethane-cyclohexane to give pure product in 93% (295 mg). ¹H-NMR (400, DMSO-d6) δ 7.69 (4H, d+d, J₁=J₂=8.4 Hz), 7.58 (2H, d, J=8.4 Hz), 7.57 (1H, s), 7.32 (1H, s), 6.97 (2H, d, J=8.4 Hz), and 1.50 (6H, s) ppm. 13C-NMR (400, DMSO-d6) δ 193.7, 175.4, 159.8, 137.5, 136.7, 132.1, 131.6, 130.0, 129.0, 118.5, 80.9, and 25.4 ppm.

Method E (N-methyl amides). Preparation of 2-(4-(4-chlorobenzoyl)phenoxy)-N,2-dimethylpropanamide (MA). Aqueous (20 ml) solution of methylamine hydrochloride (675 mg; 10 mmol) and sodium carbonate (530 mg; 5 mmol) were mixed with tetrahydrofuran (20 ml) solution of acid chloride prepared as explain above from fenofibric acid (FFA, 318 mg; 1 mmol) and stirred at room temperature for two hours. Dichloromethane was added (50 ml) and organic layer washed with water (3×50 ml), 5% sodium carbonate (50 ml) and dried over anhydrous sodium carbonate. After evaporation of the oily residue was mixed with cyclohexane (5 ml) and left at room temperature overnight. Formed white needles were separated by filtration and dried at 60° C. under vacuum to give pure product in 90% (300 mg). ¹H-NMR (400, DMSO-d₆) δ 8.09 (1H, q, J=4.8 Hz), 7.67 (2H, d, J=8.8 Hz), 7.66 (2H, d, J=8.8 Hz), 7.56 (2H, d, J=8.8 Hz), 6.93 (2H, d, J=8.8 Hz), 2.58 (3H, d, J=4.8 Hz), and 1.47 (6H, s) ppm. ¹³C-NMR (400, DMSO-d6) δ 194.0, 174.0, 159.6, 137.6, 136.5, 132.2, 131.6, 130.2, 129.1, 118.9, 81.2, 30.7, and 25.4 ppm.

Method F (N,N-dimethyl amides). Preparation 2-(4-(4-chlorobenzoyl)phenoxy)-N,N,2-trimethylpropanamide (DMA). Mixture of 40% dimethylamine in water (10 ml; 88 mmol) and the acid chloride of fenofibric acid (FFA, 1 mmol) was stirred at room temperature for two hours. Reaction mixture was worked up as described above. Product was purified by crystallization from cyclohexane. Isolated yield 89% (310 mg). ¹H-NMR (400, DMSO-d6) δ 7.71 (2H, d, J=8.8 Hz), 7.69 (2H, d, J=8.8 Hz), 6.89 (2H, d, J=8.8 Hz), 3.02 (3H, s), 2.82 (3H, s), and 1.59 ppm. ¹³C-NMR (400, DMSO-d₆) δ 193.9, 171.5, 159.6, 137.5, 136.7, 132.6, 131.6, 130.1, 129.1, 116.8, 81.6, 37.3, and 25.9 ppm.

Method G (pH neutral nitrogen substituted phenoxyacetamides). Preparation of 2-(4-(4-chlorophenoxy)phenoxy)-N,N-bis(2-hydroxyethyl)-2-methylpropanamide (HR5). Dry dichloromethane (20 ml) solution of acid 1k (306 mg; 1 mmol), oxalyl chloride (0.260 ml; 380 mg; 3 mmol) and two drops of DMF was stirred at room temperature for 4 hours. Solvent was evaporated, and residue was dissolved in dichloromethane and mixed with mixture of tetrahydrofuran (10 ml) with diethanolamine (160 mg; 1.5 mmol) and water (10 ml) with sodium carbonate (212 mg; 2 mmol). Resulting mixture was stirred at room temperature 2 hours and solvent was evaporated under reduced pressure. Solid residue was mixed with dichloromethane (100 ml) and water (100 ml). Water layer was discarded, and organic layer was washed with 5% hydrochloric acid (3×50 ml), 5% sodium carbonate (3×50 ml), water (3×50 ml) and dried over anhydrous sodium carbonate. Solvent was evaporated under reduced pressure to give pure product in 84% (330 mg) yield. ¹H-NMR (300 MHz, CDCl₃) δ 6.92 (6H, m), 6.82 (2H, d, J=9.3 Hz), 3.92 (4H, m), 3.66 (2H, t, J=5.8 Hz), 3.60 (2H, t, J=5.8 Hz), and 1.65 (6H) ppm.

Method H (basic nitrogen substituted aryloxyacetamides). Preparation of 2-(4-(4-chlorobenzoyl)phenoxy)-N-(2-(diethylamino)ethyl)-2-methylpropanamide (HR32). Tetrahydrofuran (20 ml) mixture of FFA (318.75; 1 mmol), oxalyl chloride (0.26 ml; 3 mmol), and two drops of DMF was stirred at room temperature for 2 hours. Solvent was evaporated, and solid residue was mixed with dry tetrahydrofuran (20 ml) and cooled down to 5° C. This solution was mixed at 5° C. with ice cold water solution (20 ml) of sodium carbonate (212 mg; 2 mol) and N,N-diethylethylenediamine (127 mg; 1.1 mmol). Resulting mixture was stirred at room temperature for two hours and evaporated to an oily residue. This residue was mixed with dichloromethane (100 ml) and 5% sodium carbonate (50 ml). Water layer was discarded, and organic layer was extensively washed with water (10×50 ml) and dried over anhydrous sodium carbonate. After solvent evaporation product was further purified by silica gel filtration with 5% triethylamine in ethyl acetate as solvent. Isolated yield 75% (317 mg). ¹H-NMR (400 MHz, CDCl₃) δ 7.73 (2H, d, J=8.8 Hz), 7.69 (2H, d, J=8.4 Hz), 7.45 (2H, d, J=8.4 Hz), 7.730 (1H, broad s), 6.95 (2H, d, J=8.8 Hz), 3.31 (2H, q, J=6.0 Hz), 2.49 (2H, t, J=6.0 Hz), 2.41 (4H, q, J=7.2 Hz), 1.61 (6H, s), and 0.87 (6H, t, J=7.2 Hz) ppm. ¹³C-NMR (300, CDCl₃) δ 194.1, 173.8, 158.9, 138.5, 136.2, 131.8, 131.1, 128.5, 119.4, 119.0, 81.7, 51.3, 46.6, 36.8, 25.1, and 11.6 ppm.

Method I (catalytic hydrogenation of benzoylphenoxyacetamides) Preparation of 2-(4-(cyclohexylmethyl)phenoxy)-N-(2-hydroxyethyl)-N,2-dimethylpropanamide (HR1). Ethanol (50 ml) suspension of amide HR40 (190 mg; 0.5 mmol) and 3% Pd/C (150 mg) was stirred at room temperature overnight under atmospheric hydrogen pressure. The catalyst was removed by filtration, solvent was evaporated under reduce pressure to give pure product in 96% (320 mg) isolated yield. ¹H-NMR (400, CDCl₃) δ 6.99 (2H, d, J=8.4 Hz), 6.74 (2H, d, J=8.4 Hz, 3.79 (2H, t, J=5.2 Hz), 3.54 (2H, t, J=5.2 Hz), 3.24 (3H, s), 3.21 (1H, m), 2.39 (2H, d, J=7.6 Hz), 1.64 (4H, m), 1.62 (6H, s), 1.45 (2H, m), 1.17 (2H, m), and 0.98 (2H, m) ppm

Method J (sodium borohydride—trifluoracetic acid reduction of benzolphenoxyacetamide carbonyl group). Preparation of 2-(4-(4-chlorobenzyl)phenoxy)-N-(2-hydroxyethyl)-N,2-dimethylpropanamide (HR2). Dichloromethane (30 ml) suspension of HR40 (190 mg; 0.5 mmol) and fine grinded sodium borohydride (110 mg; 3 mmol) was kept at −5° C. for one hour. Into this stirring suspension at −5° C. slowly dropwise trifluoracetic acid (15 ml) was added in period of half an hour. Resulting suspension was then stirred at 0° C. for one hour and at room temperature for additional two hours. The suspension was filtered, solid was discarded and dichloromethane filtrate was washed with 5% sodium carbonate (3×30 ml), dried over anhydrous sodium carbonate and evaporated to oily residue to give crude product. The product was purified by silica gel column chromatography with ethyl acetate-dichloromethane (7:3). Isolated yield 72%. ¹H-NMR (400, CDCl₃) δ 7.23 (2H, d, J=8.4 Hz), 7.08 (2H, d, J=8.4 Hz), 7.01 (2H, d, J=8.4 Hz), 6.76 (2H, d, J=8.4 Hz), 3.85 (2H, s), 3.78 (2H, q, J=4.8 Hz), 3.53 (2H, t, J=4.8 Hz), 3.21 (3H, s), 2.73 (1H, t, J=5.2 Hz), and 1.62 (6H, s) pppm.

Method K (hydrochloric salts of basic phenoxyacetamide). Preparation of 4-{2-[4-(4-chlorobenzoyl)phenoxy]-2-methylpropanoyl}6-8,13-20,24-26,28,34,38-1-methylpiperazin-1-ium chloride (HR35). Mixture of concentrated hydrochloric (3 ml) and HR34 (200 mg; 0.5 mmol) was sonicated at room temperature for five minutes. Clear water solution was left under stream of nitrogen in hood for several hours to result in white powder. White powder was dissolved in dichloromethane (10 ml) and dried over 4 Å molecular sieve overnight. The solvent was evaporated, and white residue was dried in vacuum to give 208 mg (95%). ¹H-NMR (CDCl₃) δ 13.20 (1H, broad s), 7.75 (2H, d. J=8.8 Hz), 7.71 (2H, d, J=8.0 Hz), 7.56 (2H, d, J=8.0 Hz), 6.90 (2H, d, J=8.8 Hz), 4.78 (2H, d, J=13.2 Hz), 3.93 (1H, t, J=12.8 Hz), 3.52 (1H, t, J=13.2 Hz), 3.45 (1H, d, J=12.4 Hz), 3.27 (1H, d, J=10.0 Hz), 2.63 (3H, d, J=4 Hz), 2.53 (1H, q, J=10.0 Hz), 1.98 (1H, q, J=9.6 Hz) and 1.71 (6H, s) ppm.

Method L (ammonium salts of basic phenoxyacetamides. Preparation of 4-{2-[4-(4-chlorobenzoyl)phenoxy]-2-methylpropanoyl}-1,1-dimethylpiperazin-1-ium iodide (HR36). Acetone (20 ml) solution of HR34 (100 mg; 0.25 mmol) and methyl Iodide (141 mg; 0.06 ml; 1 mmol) was left in dark at room temperature for three days (˜70 hours). During the reaction white solid precipitate forms. Solid product was separated by filtration. Washed with acetone (3×3 ml) and dried at 110° C. for two hours to give pure product in 96% (130 mg). ¹H-NMR (DMSO-d6) δ 7.73 (2H, d, J=8.8 Hz), 7.71 (2H, d, J=8.8 Hz), 7.60 (2H, d, J=7.6 Hz), 6.93 (2H, d, J=8.4). 4.06 (2H, broad singlet), 3.86 (2H, broad singlet), 3.30 (2H, broad singlet), 3.25 (2H, broad singlet), 3.12 (6H, s) and 1.60 (3H, s) ppm. 13C-NMR (DMSO-d6) δ 193.7, 170.7, 159.1, 137.6, 136.5, 132.7, 131.7, 130.6, 129.1, 117.7, 81.8, 60.7, 51.2, and 26.2 ppm.

### REFERENCES CITED IN THIS EXAMPLE

1 Di Carlo, D. T., Cagnazzo, F., Benedetto, N., Morganti, R. & Perrini, P. Multiple high-grade gliomas: epidemiology, management, and outcome. A systematic review and meta-analysis. Neurosurg Rev 42, 263-275(2019).
2 Nakada, M. et al. Molecular targets of glioma invasion. Cell Mol Life Sci 64, 458-478 (2007).
3 Paolillo, M., Boselli, C. & Schinelli, S. Glioblastoma under Siege: An Overview of Current Therapeutic Strategies. Brain Sci 8 (2018).
4 Terzis, A. J., Niclou, S. P., Rajcevic, U., Danzeisen, C. & Bjerkvig, R. Cell therapies for glioblastoma. Expert opinion on biological therapy 6, 739-749 (2006). Davis, M. E. Glioblastoma: Overview of Disease and Treatment. Clin J Oncol Nurs 20, S2-8 (2016).
6 Cen, L. et al. Efficacy of protracted temozolomide dosing is limited in MGMT unmethylated GBM xenograft models. Neuro Oncol 15, 735-746(2013).
7 Fu, J. et al. Glioblastoma stem cells resistant to temozolomide-induced autophagy. Chinese medical journal 122, 1255-1259 (2009).
8 Munoz, J. L. et al. Temozolomide resistance in glioblastoma cells occurs partly through epidermal growth factor receptor-mediated induction of connexin 43. Cell death & disease 5, e1145 (2014).
9 Ghosh, D., Nandi, S. & Bhattacharjee, S. Combination therapy to checkmate Glioblastoma: clinical challenges and advances. Clin Transl Med 7, 33 (2018).
10 Ohgaki, H. & Kleihues, P. Genetic pathways to primary and secondary glioblastoma. The American journal of pathology 170, 1445-1453 (2007).
11 Desai, K., Hubben, A. & Ahluwalia, M. The Role of Checkpoint Inhibitors in Glioblastoma. Target Oncol (2019).
12 Geng, F. et al. Inhibition of SOAT1 Suppresses Glioblastoma Growth via Blocking SREBP-1-Mediated Lipogenesis. Clin Cancer Res 22, 5337-5348 (2016).
13 Drukala, J. et al. ROS accumulation and IGF-IR inhibition contribute to fenofibrate/PPARalpha-mediated inhibition of glioma cell motility in vitro. Mol Cancer 9, 159 (2010).
14 Wybieralska, E. et al. Fenofibrate attenuates contact-stimulated cell motility and gap junctional coupling in DU-145 human prostate cancer cell populations. Oncol Rep 26, 447-453 (2011).
15 Yamasaki, D. et al. Fenofibrate suppresses growth of the human hepatocellular carcinoma cell via PPARalpha-independent mechanisms. Eur J Cell Biol 90, 657-664 (2011).
16 Wilk, A. et al. Fenofibrate-induced nuclear translocation of FoxO3A triggers Bim-mediated apoptosis in glioblastoma cells in vitro. Cell Cycle 11, 2660-2671 (2012).
17 Koltai, T. Fenofibrate in cancer:mechanisms involved in anticancer activity. F1000Research 4, 1-22 (2015).
18 Grabacka, M. M. et al. Fenofibrate Induces Ketone Body Production in Melanoma and Glioblastoma Cells. Frontiers in endocrinology 7, 5(2016).
19 Wilk, A. et al. Molecular Mechanisms of Fenofibrate-Induced Metabolic Catastrophe and Glioblastoma Cell Death. Molecular and cellular biology (2014).
20 Grabacka, M. et al. Fenofibrate subcellular distribution as a rationale for the intracranial delivery through biodegradable carrier. Journal of physiology and pharmacology: an official journal of the Polish Physiological Society 66, 233-247 (2015).
21 Stalinska J, Z. E., Pianovich N A, Zapata A, Lassak A, Rak M, Dean M, Ucar-Bilyeu D, Wyczechowska D, Culicchia F, Marrero L, DelValle L, Sarkaria J, Peruzzi F, Jursic B S, Reiss K. Chemically modified variants of fenofibrate with anti-glioblastoma potential. Translational Oncology (2019).
22 Eisenbraun, E. J. & Payne, K. W. Dean-Stark apparatus modified for use with molecular sieves. Ind Eng Chem Res 38, 4521-4524 (1999).
23 Valeur, E. & Bradley, M. Amide bond formation: beyond the myth of coupling reagents. Chem Soc Rev 38, 606-631 (2009).
24 Rani, P., Pal, D., Hegde, R. R. & Hashim, S. R. Anticancer, Anti-Inflammatory, and Analgesic Activities of Synthesized 2-(Substituted phenoxy) Acetamide Derivatives. Biomed Res Int (2014).
25 Wager, T. T., Hou, X. J., Verhoest, P. R. & Villalobos, A. Moving beyond Rules: The Development of a Central Nervous System Multiparameter Optimization (CNS MPO) Approach To Enable Alignment of Druglike Properties. Acs Chem Neurosci 1, 435-449 (2010).
26 Wager, T. T., Hou, X. J., Verhoest, P. R. & Villalobos, A. Central Nervous System Multiparameter Optimization Desirability: Application in Drug Discovery. Acs Chem Neurosci 7, 767-775 (2016).
27 Rankovic, Z. CNS Drug Design: Balancing Physicochemical Properties for Optimal Brain Exposure. J Med Chem 58, 2584-2608 (2015).
28 Wang, J. M. et al. Development of Polarizable Models for Molecular Mechanical Calculations I: Parameterization of Atomic Polarizability. J Phys Chem B 115, 3091-3099 (2011).
29 Naef, R. A Generally Applicable Computer Algorithm Based on the Group Additivity Method for the Calculation of Seven Molecular Descriptors: Heat of Combustion, Log PO/W, Log S, Refractivity, Polarizability, Toxicity and Log BB of Organic Compounds; Scope and Limits of Applicability. Molecules 20, 18279-18351 (2015).
30 Cha, H. J., Muller, R. T. & Pos, K. M. Switch-Loop Flexibility Affects Transport of Large Drugs by the Promiscuous AcrB Multidrug Efflux Transporter. Antimicrob Agents Ch 58, 4767-4772 (2014).
31 Savjani, K. T., Gajjar, A. K. & Savjani, J. K. Drug solubility: importance and enhancement techniques. ISRN Pharm 2012, 195727 (2012).
32 Vilar, S., Chakrabarti, M. & Costanzi, S. Prediction of passive blood-brain partitioning: Straightforward and effective classification models based on in silico derived physicochemical descriptors. J Mol Graph Model 28, 899-903 (2010).
33 Piiper, J. & Worth, H. Value and limits of Graham's law for prediction of diffusivities of gases in gas mixtures. Respir Physiol 41, 233-240 (1980).
34 Shinoda, W. Permeability across lipid membranes. Bba-Biomembranes 1858, 2254-2265 (2016).
35 Kumar, A. & Zhang, K. Y. J. Advances in the Development of Shape Similarity Methods and Their Application in Drug Discovery. Front Chem 6 (2018).
36 Shah, P. & Westwell, A. D. The role of fluorine in medicinal chemistry. J Enzym Inhib Med Ch 22, 527-540 (2007).
37 Williams, H. D. et al. Strategies to Address Low Drug Solubility in Discovery and Development. Pharmacol Rev 65, 315-499 (2013).
38 Lawson, A. D. G., MacCoss, M. & Heer, J. P. Importance of Rigidity in Designing Small Molecule Drugs To Tackle Protein-Protein Interactions (PPIs) through Stabilization of Desired Conformers. J Med Chem 61, 4283-4289(2018).
39 Liston, D. R. & Davis, M. Clinically Relevant Concentrations of Anticancer Drugs: A Guide for Nonclinical Studies. Clinical Cancer Research 23, 3489-3498 (2017).
40 Mosmann, T. Rapid colorimetric assay for cellular growth and survival: application to proliferation and cytotoxicity assays. J Immunol Methods 65, 55-63 (1983).

### Example 4

Non-limiting examples of anti-cancer compounds comprises those described in Example 4.

| Compound | Structure | Formula | MW |
|---|---|---|---|
| PP1 | 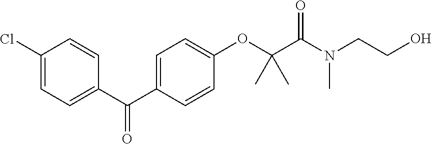 [SMILES: CN(CCO)C(=O)C(C)(C)Oc1ccc(C(=O)c2ccc(Cl)cc2)cc1] | C₂₀H₂₂ClNO₄ | 375.85 |
| PP2 | 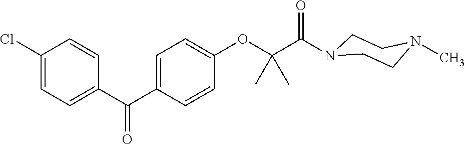 [SMILES: CN1CCN(C(=O)C(C)(C)Oc2ccc(C(=O)c3ccc(Cl)cc3)cc2)CC1] | C₂₂H₂₅ClN₂O₃ | 400.90 |
| PP2HCl | 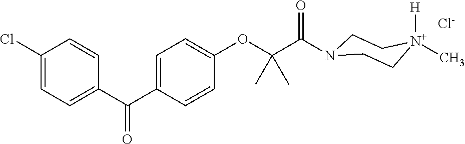 [SMILES: C[NH+]1CCN(C(=O)C(C)(C)Oc2ccc(C(=O)c3ccc(Cl)cc3)cc2)CC1 ; [Cl-]] | C₂₂H₂₆Cl₂N₂O₃ | 437.36 |
| PP2Mel | 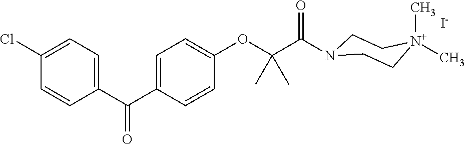 [SMILES: CC(C)(Oc1ccc(C(=O)c2ccc(Cl)cc2)cc1)C(=O)N1CC[N+](C)(C)CC1 ; [I-]] | C₂₃H₂₈ClIN₂O₃ | 542.84 |
| PP3 | 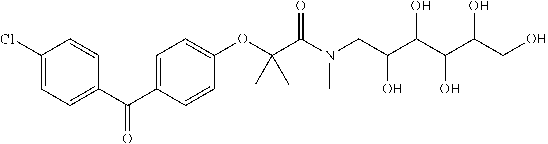 [SMILES: CN(CC(O)C(O)C(O)C(O)CO)C(=O)C(C)(C)Oc1ccc(C(=O)c2ccc(Cl)cc2)cc1] | C₂₄H₃₀ClNO₈ | 495.95 |
| PP4 | 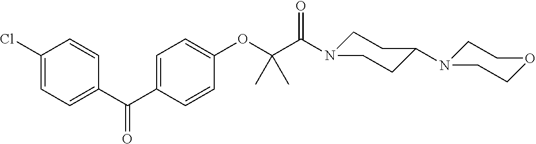 [SMILES: CC(C)(Oc1ccc(C(=O)c2ccc(Cl)cc2)cc1)C(=O)N1CCC(N2CCOCC2)CC1] | C₂₆H₃₁ClN₂O₄ | 470.99 |
| PP4HCl | 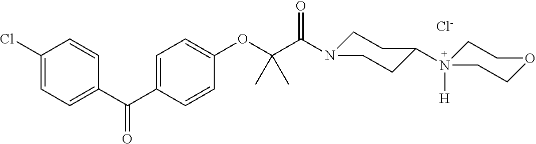 | C₂₆H₃₂Cl₂N₂O₄ | 507.45 |
| PP5 | 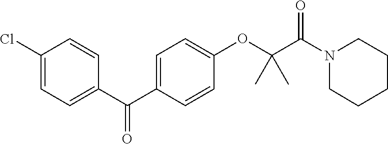 [SMILES: CC(C)(Oc1ccc(C(=O)c2ccc(Cl)cc2)cc1)C(=O)N1CCCCC1] | C₂₂H₂₄ClNO₃ | 385.88 |
| PP6 | 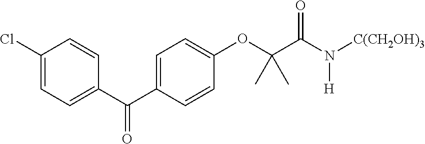 | C₂₁H₂₄ClNO₆ | 421.87 |
| PP7 | 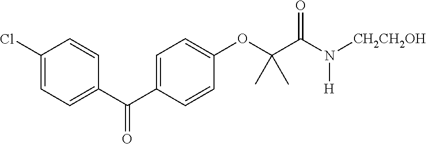 | C₁₉H₂₀ClNO₄ | 361.82 |
| PP8 | 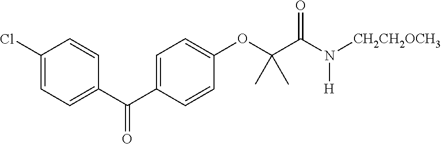 | C₂₀H₂₂ClNO₄ | 375.85 |
| PP9 | 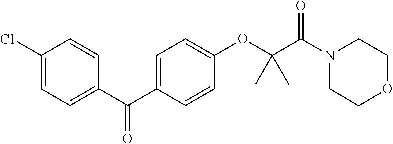 [SMILES: CC(C)(Oc1ccc(C(=O)c2ccc(Cl)cc2)cc1)C(=O)N1CCOCC1] | C₂₁H₂₂ClNO₄ | 387.86 |
| PP10 | 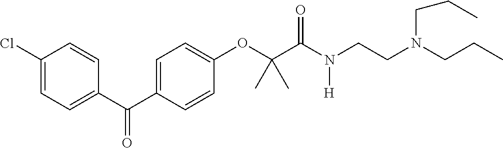 [SMILES: CCCN(CCC)CCNC(=O)C(C)(C)Oc1ccc(C(=O)c2ccc(Cl)cc2)cc1] | C₂₅H₃₃ClN₂O₃ | 444.99 |
| PP11 | 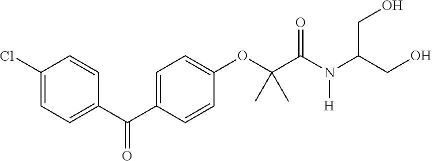 [SMILES: CC(C)(Oc1ccc(C(=O)c2ccc(Cl)cc2)cc1)C(=O)NC(CO)CO] | C₂₀H₂₂ClNO₅ | 391.85 |
| PP12 | 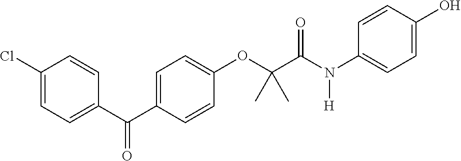 [SMILES: CC(C)(Oc1ccc(C(=O)c2ccc(Cl)cc2)cc1)C(=O)Nc1ccc(O)cc1] | C₂₃H₂₀ClNO₄ | 409.86 |
| PP13 | 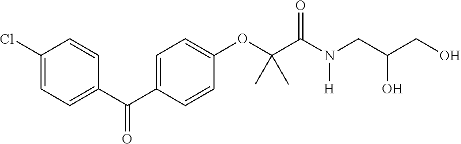 [SMILES: CC(C)(Oc1ccc(C(=O)c2ccc(Cl)cc2)cc1)C(=O)NCC(O)CO] | C₂₀H₂₂ClNO₅ | 391.85 |
| PP14 | 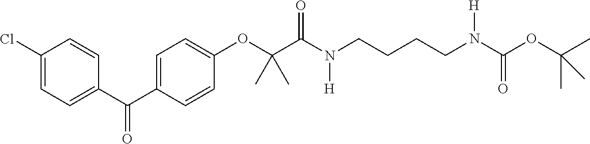 [SMILES: CC(C)(C)OC(=O)NCCCCNC(=O)C(C)(C)Oc1ccc(C(=O)c2ccc(Cl)cc2)cc1] | C₂₆H₃₃ClN₂O₅ | 489.00 |
| PP15 | 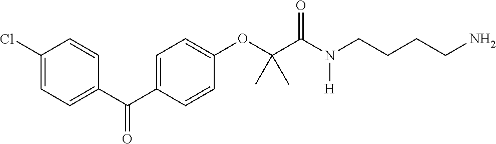 [SMILES: CC(C)(Oc1ccc(C(=O)c2ccc(Cl)cc2)cc1)C(=O)NCCCCN] | C₂₁H₂₅ClN₂O₃ | 388.89 |
| PP16 | 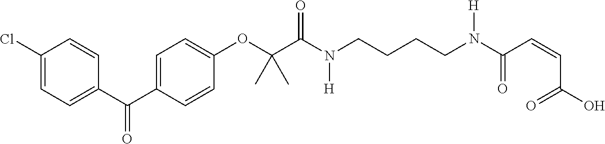 [SMILES: CC(C)(Oc1ccc(C(=O)c2ccc(Cl)cc2)cc1)C(=O)NCCCCNC(=O)/C=C\C(=O)O] | C₂₅H₂₇ClN₂O₆ | 486.94 |
| PP17 | 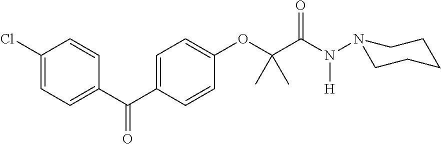 [SMILES: CC(C)(Oc1ccc(C(=O)c2ccc(Cl)cc2)cc1)C(=O)NN1CCCCC1] | C₂₂H₂₅ClN₂O₃ | 400.90 |
| PP18 | 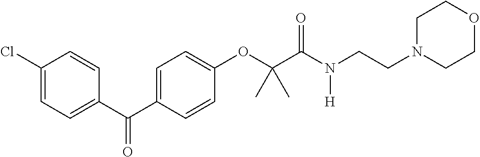 [SMILES: CC(C)(Oc1ccc(C(=O)c2ccc(Cl)cc2)cc1)C(=O)NCCN1CCOCC1] | C₂₃H₂₇ClN₂O₄ | 430.92 |
| PP19 | 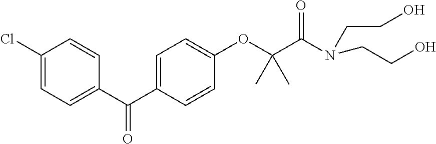 [SMILES: CC(C)(Oc1ccc(C(=O)c2ccc(Cl)cc2)cc1)C(=O)N(CCO)CCO] | C₂₁H₂₄ClNO₅ | 405.87 |
| PP20 | 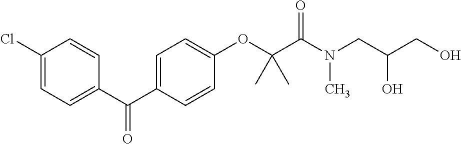 [SMILES: CN(CC(O)CO)C(=O)C(C)(C)Oc1ccc(C(=O)c2ccc(Cl)cc2)cc1] | C₂₁H₂₄ClNO₅ | 405.87 |
| PP21 | 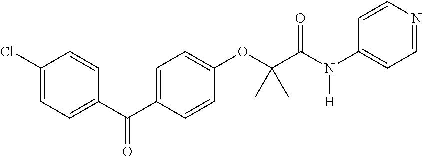 [SMILES: CC(C)(Oc1ccc(C(=O)c2ccc(Cl)cc2)cc1)C(=O)Nc1ccncc1] | C₂₂H₁₉ClN₂O₃ | 394.85 |
| PP22 | 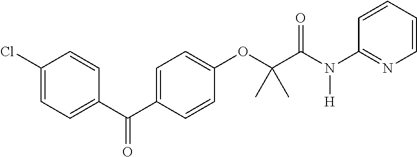 [SMILES: CC(C)(Oc1ccc(C(=O)c2ccc(Cl)cc2)cc1)C(=O)Nc1ccccn1] | C₂₂H₁₉ClN₂O₃ | 394.85 |
| PP23 | 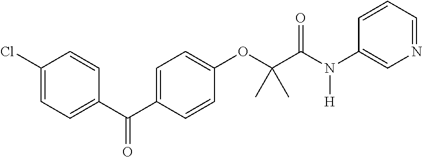 [SMILES: CC(C)(Oc1ccc(C(=O)c2ccc(Cl)cc2)cc1)C(=O)Nc1cccnc1] | C₂₂H₁₉ClN₂O₃ | 394.85 |
| PP24 | 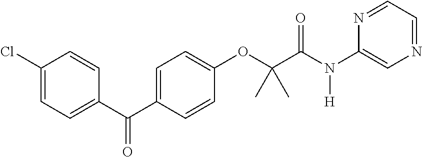 [SMILES: CC(C)(Oc1ccc(C(=O)c2ccc(Cl)cc2)cc1)C(=O)Nc1cnccn1] | C₂₁H₁₈ClN₃O₃ | 395.84 |
| PP25 | 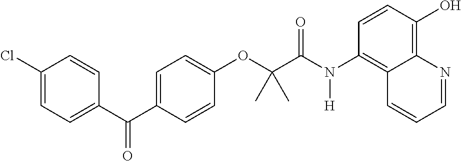 [SMILES: CC(C)(Oc1ccc(C(=O)c2ccc(Cl)cc2)cc1)C(=O)Nc1ccc(O)c2ncccc12] | C₂₆H₂₁ClN₂O₄ | 460.91 |
| PP26 | 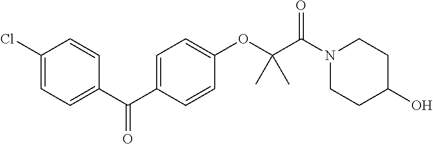 [SMILES: CC(C)(Oc1ccc(C(=O)c2ccc(Cl)cc2)cc1)C(=O)N1CCC(O)CC1] | C₂₂H₂₄ClNO₄ | 401.88 |
| PP27 | 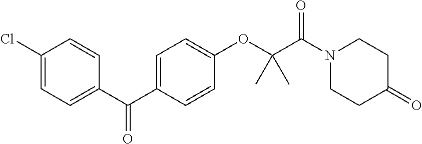 [SMILES: CC(C)(Oc1ccc(C(=O)c2ccc(Cl)cc2)cc1)C(=O)N1CCC(=O)CC1] | C₂₂H₂₂ClNO₄ | 399.87 |
| PP28 | 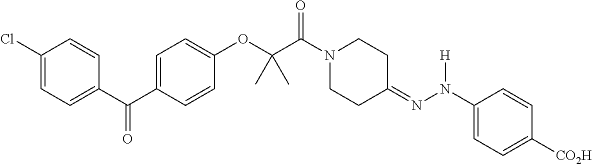 [SMILES: CC(C)(Oc1ccc(C(=O)c2ccc(Cl)cc2)cc1)C(=O)N1CCC(=NNc2ccc(C(=O)O)cc2)CC1] | C₂₉H₂₈ClN₃O₅ | 534.00 |
| PP29 | 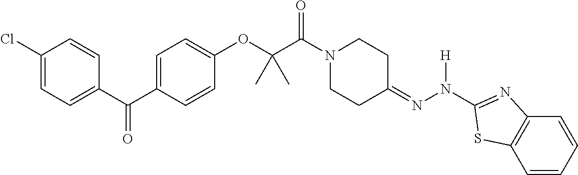 [SMILES: CC(C)(Oc1ccc(C(=O)c2ccc(Cl)cc2)cc1)C(=O)N1CCC(=NNc2nc3ccccc3s2)CC1] | C₂₉H₂₇ClN₄O₃S | 547.07 |
| PP30 | 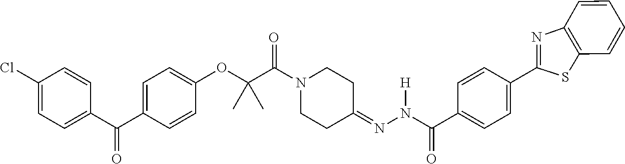 [SMILES: CC(C)(Oc1ccc(C(=O)c2ccc(Cl)cc2)cc1)C(=O)N1CCC(=NNC(=O)c2ccc(-c3nc4ccccc4s3)cc2)CC1] | C₃₆H₃₁ClN₄O₄S | 651.17 |
| PP31 | 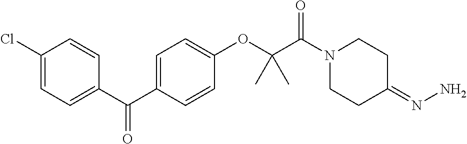 [SMILES: CC(C)(Oc1ccc(C(=O)c2ccc(Cl)cc2)cc1)C(=O)N1CCC(=NN)CC1] | C₂₂H₂₄ClN₃O₃ | 413.90 |
| PP32 | 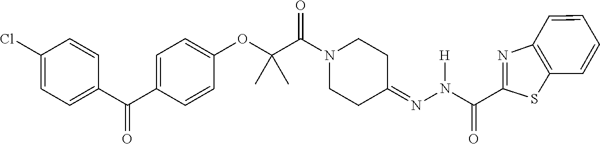 [SMILES: CC(C)(Oc1ccc(C(=O)c2ccc(Cl)cc2)cc1)C(=O)N1CCC(=NNC(=O)c2nc3ccccc3s2)CC1] | C₃₀H₂₇ClN₄O₄S | 575.08 |
| PP33 | 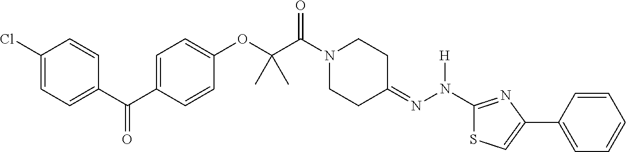 [SMILES: CC(C)(Oc1ccc(C(=O)c2ccc(Cl)cc2)cc1)C(=O)N1CCC(=NNc2nc(-c3ccccc3)cs2)CC1] | C₃₁H₂₉ClN₄O₃S | 573.11 |
| PP34 | 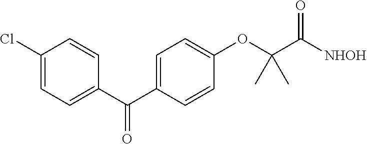 | C₁₇H₁₆ClNO₄ | 333.77 |
| PP35 | 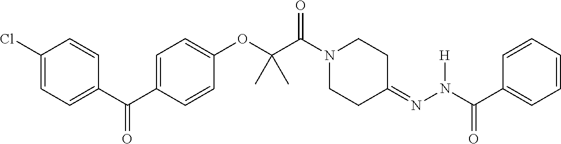 [SMILES: CC(C)(Oc1ccc(C(=O)c2ccc(Cl)cc2)cc1)C(=O)N1CCC(=NNC(=O)c2ccccc2)CC1] | C₂₉H₂₈ClN₃O₄ | 518.00 |
| PP36 | 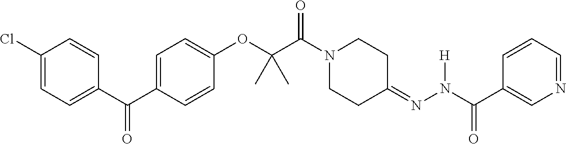 [SMILES: CC(C)(Oc1ccc(C(=O)c2ccc(Cl)cc2)cc1)C(=O)N1CCC(=NNC(=O)c2cccnc2)CC1] | C₂₈H₂₇ClN₄O₄ | 518.99 |
| PP37 | 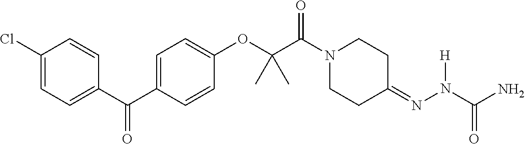 [SMILES: CC(C)(Oc1ccc(C(=O)c2ccc(Cl)cc2)cc1)C(=O)N1CCC(=NNC(N)=O)CC1] | C₂₃H₂₅ClN₄O₄ | 456.92 |
| PP38 | 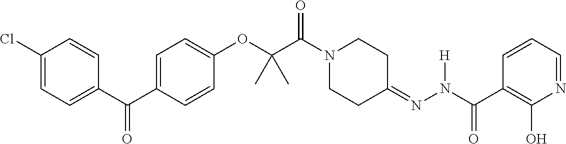 [SMILES: CC(C)(Oc1ccc(C(=O)c2ccc(Cl)cc2)cc1)C(=O)N1CCC(=NNC(=O)c2cccnc2O)CC1] | C₂₈H₂₇ClN₄O₅ | 534.99 |
| PP39 | 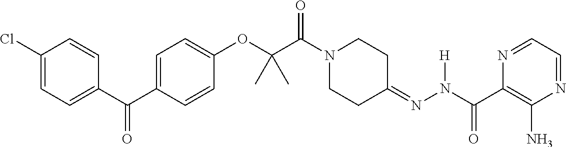 [SMILES: CC(C)(Oc1ccc(C(=O)c2ccc(Cl)cc2)cc1)C(=O)N1CCC(=NNC(=O)c2nccnc2N)CC1] | C₂₇H₂₇ClN₆O₄ | 534.99 |
| PP40 | 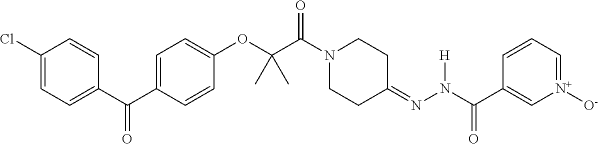 [SMILES: CC(C)(Oc1ccc(C(=O)c2ccc(Cl)cc2)cc1)C(=O)N1CCC(=NNC(=O)c2ccc[n+]([O-])c2)CC1] | C₂₈H₂₇ClN₄O₅ | 534.99 |
| PP41 | 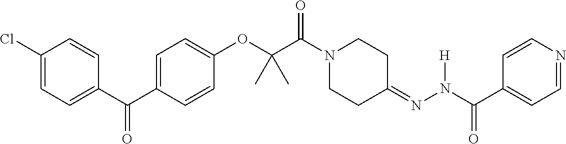 [SMILES: CC(C)(Oc1ccc(C(=O)c2ccc(Cl)cc2)cc1)C(=O)N1CCC(=NNC(=O)c2ccncc2)CC1] | C₂₈H₂₇ClN₄O₄ | 518.99 |
| PP42 | 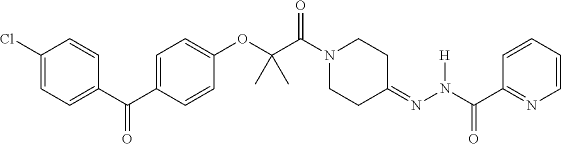 [SMILES: CC(C)(Oc1ccc(C(=O)c2ccc(Cl)cc2)cc1)C(=O)N1CCC(=NNC(=O)c2ccccn2)CC1] | C₂₈H₂₇ClN₄O₄ | 518.99 |
| PP43 | 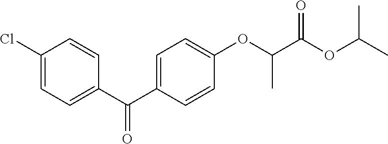 [SMILES: CC(C)OC(=O)C(C)Oc1ccc(C(=O)c2ccc(Cl)cc2)cc1] | C₁₉H₁₉ClO₄ | 346.80 |
| PP44 | 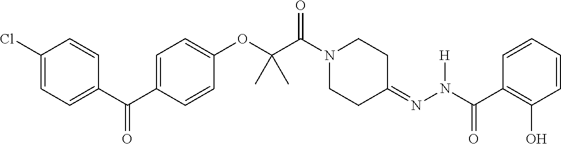 [SMILES: CC(C)(Oc1ccc(C(=O)c2ccc(Cl)cc2)cc1)C(=O)N1CCC(=NNC(=O)c2ccccc2O)CC1] | C₂₉H₂₈ClN₃O₅ | 534.00 |
| PP45 | 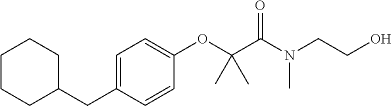 [SMILES: CN(CCO)C(=O)C(C)(C)Oc1ccc(CC2CCCCC2)cc1] | C₂₀H₃₁NO₃ | 333.47 |
| PP46 | 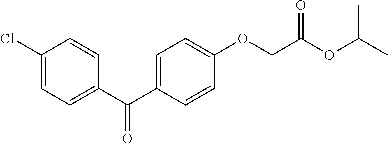 [SMILES: CC(C)OC(=O)COc1ccc(C(=O)c2ccc(Cl)cc2)cc1] | C₁₈H₁₇ClO₄ | 332.78 |
| PP47 | 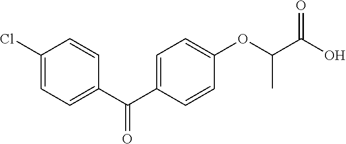 [SMILES: CC(Oc1ccc(C(=O)c2ccc(Cl)cc2)cc1)C(=O)O] | C₁₆H₁₃ClO₄ | 304.72 |
| PP48 | 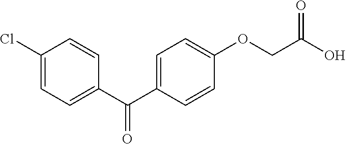 [SMILES: O=C(O)COc1ccc(C(=O)c2ccc(Cl)cc2)cc1] | C₁₅H₁₁ClO₄ | 290.70 |
| PP49 | 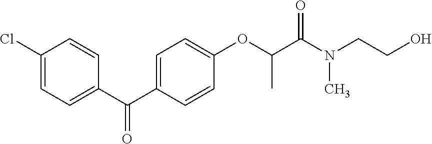 [SMILES: CC(Oc1ccc(C(=O)c2ccc(Cl)cc2)cc1)C(=O)N(C)CCO] | C₁₉H₂₀ClNO₄ | 361.82 |
| PP50 | 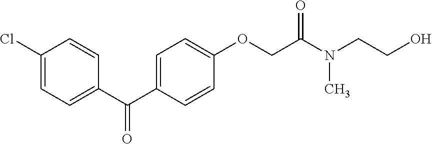 [SMILES: CN(CCO)C(=O)COc1ccc(C(=O)c2ccc(Cl)cc2)cc1] | C₁₈H₁₈ClNO₄ | 347.79 |
| PP51 | 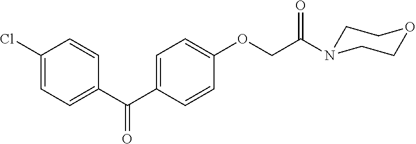 [SMILES: O=C(c1ccc(Cl)cc1)c1ccc(OCC(=O)N2CCOCC2)cc1] | C₁₉H₁₈ClNO₄ | 359.80 |
| PP52 | 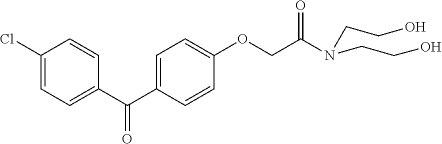 [SMILES: O=C(c1ccc(Cl)cc1)c1ccc(OCC(=O)N(CCO)CCO)cc1] | C₁₉H₂₀ClNO₅ | 377.82 |
| PP53 | 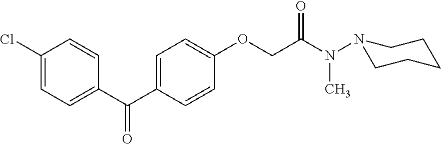 [SMILES: CN(C(=O)COc1ccc(C(=O)c2ccc(Cl)cc2)cc1)N1CCCCC1] | C₂₁H₂₃ClN₂O | 386.87 |
| PP54 | 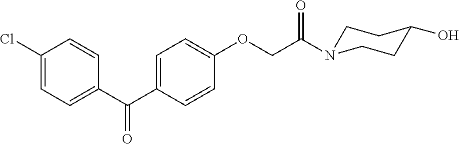 [SMILES: O=C(c1ccc(Cl)cc1)c1ccc(OCC(=O)N2CCC(O)CC2)cc1] | C₂₀H₂₀ClNO₄ | 373.83 |
| PP55 | 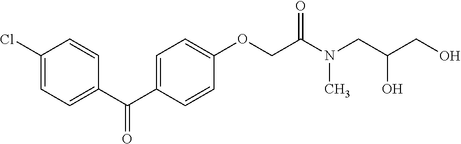 [SMILES: CN(CC(O)CO)C(=O)COc1ccc(C(=O)c2ccc(Cl)cc2)cc1] | C₁₉H₂₀ClNO₅ | 377.82 |
| PP56 | 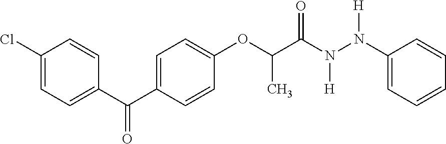 [SMILES: CC(Oc1ccc(C(=O)c2ccc(Cl)cc2)cc1)C(=O)NNc1ccccc1] | C₂₂H₁₉ClN₂O₃ | 394.85 |
| PP57 | 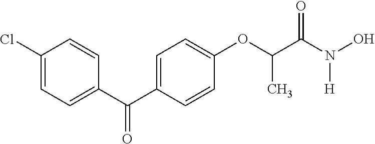 [SMILES: CC(Oc1ccc(C(=O)c2ccc(Cl)cc2)cc1)C(=O)NO] | C₁₆H₁₄ClNO₄ | 319.74 |
| PP58 | 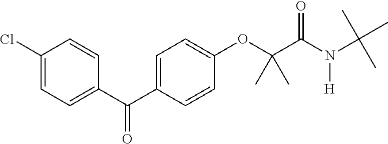 [SMILES: CC(C)(C)NC(=O)C(C)(C)Oc1ccc(C(=O)c2ccc(Cl)cc2)cc1] | C₂₁H₂₄ClNO₃ | 373.87 |
| PP59 | 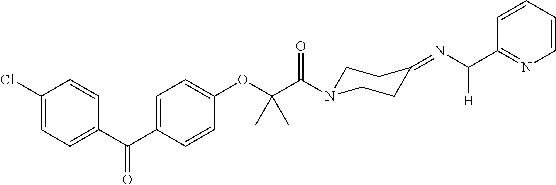 [SMILES: CC(C)(Oc1ccc(C(=O)c2ccc(Cl)cc2)cc1)C(=O)N1CCC(=NCc2ccccn2)CC1] | C₂₇H₂₇ClN₄O₃ | 490.98 |
| PP60 | 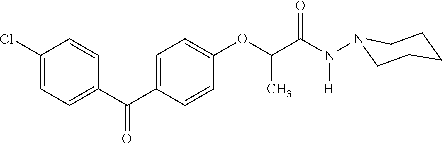 [SMILES: CC(Oc1ccc(C(=O)c2ccc(Cl)cc2)cc1)C(=O)NN1CCCCC1] | C₂₁H₂₃ClN₂O₃ | 386.87 |
| PP61 | 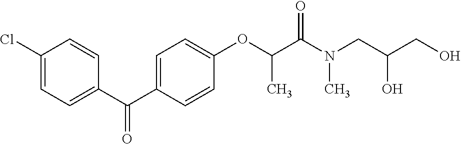 [SMILES: CC(Oc1ccc(C(=O)c2ccc(Cl)cc2)cc1)C(=O)N(C)CC(O)CO] | C₂₀H₂₂ClNO₅ | 391.85 |
| PP62 | 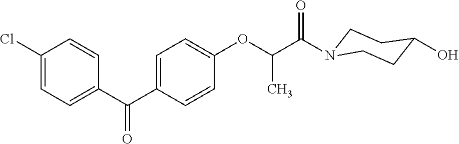 [SMILES: CC(Oc1ccc(C(=O)c2ccc(Cl)cc2)cc1)C(=O)N1CCC(O)CC1] | C₂₁H₂₂ClNO₄ | 387.86 |
| PP63 | 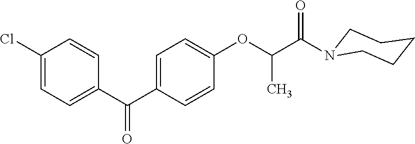 [SMILES: CC(Oc1ccc(C(=O)c2ccc(Cl)cc2)cc1)C(=O)N1CCCCC1] | C₂₁H₂₂ClNO₃ | 371.86 |
| PP64 | 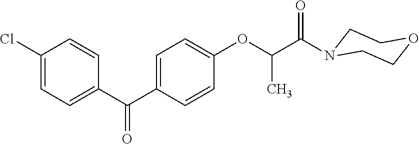 [SMILES: CC(Oc1ccc(C(=O)c2ccc(Cl)cc2)cc1)C(=O)N1CCOCC1] | C₂₀H₂₀ClNO₄ | 373.83 |
| PP65 | 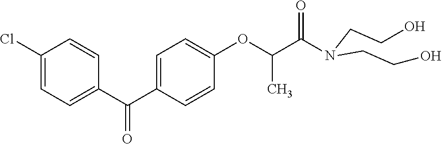 [SMILES: CC(Oc1ccc(C(=O)c2ccc(Cl)cc2)cc1)C(=O)N(CCO)CCO] | C₂₀H₂₂ClNO₅ | 391.85 |
| PP66 | 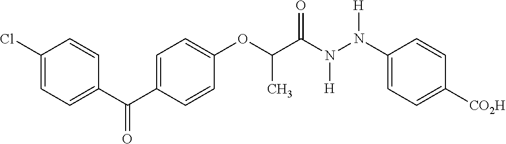 [SMILES: CC(Oc1ccc(C(=O)c2ccc(Cl)cc2)cc1)C(=O)NNc1ccc(C(=O)O)cc1] | C₂₃H₁₉ClN₂O₅ | 438.86 |
| PP67 | 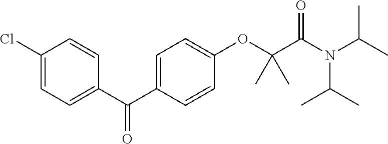 [SMILES: CC(C)N(C(=O)C(C)(C)Oc1ccc(C(=O)c2ccc(Cl)cc2)cc1)C(C)C] | C₂₃H₂₈ClNO₃ | 401.93 |
| PP68 | 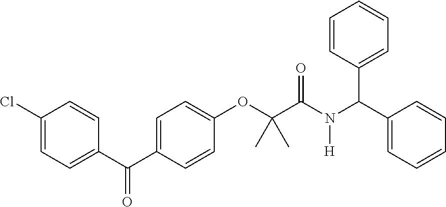 [SMILES: CC(C)(Oc1ccc(C(=O)c2ccc(Cl)cc2)cc1)C(=O)NC(c1ccccc1)c1ccccc1] | C₃₀H₂₆ClNO₃ | 483.99 |
| PP69 | 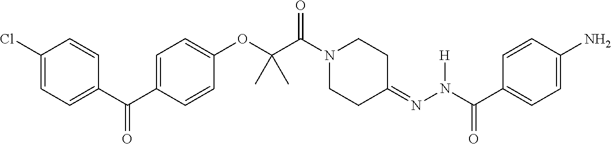 [SMILES: CC(C)(Oc1ccc(C(=O)c2ccc(Cl)cc2)cc1)C(=O)N1CCC(=NNC(=O)c2ccc(N)cc2)CC1] | C₂₉H₂₉ClN₄O₄ | 533.02 |
| PP70 | 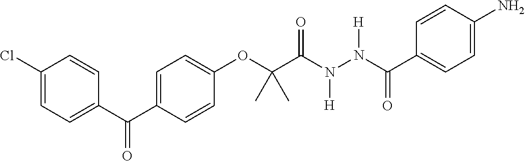 [SMILES: CC(C)(Oc1ccc(C(=O)c2ccc(Cl)cc2)cc1)C(=O)NNC(=O)c1ccc(N)cc1] | C₂₄H₂₂ClN₃O₄ | 451.90 |
| PP71 | 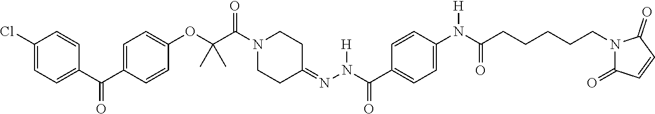 [SMILES: CC(C)(Oc1ccc(C(=O)c2ccc(Cl)cc2)cc1)C(=O)N1CCC(=NNC(=O)c2ccc(NC(=O)CCCCCN3C(=O)C=CC3=O)cc2)CC1] | C₃₉H₄₀ClN₅O₇ | 726.22 |
| PP72 | 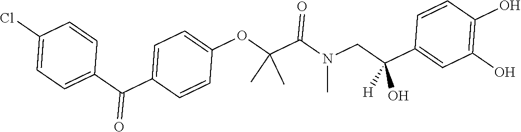 [SMILES: CN(C[C@H](O)c1ccc(O)c(O)c1)C(=O)C(C)(C)Oc1ccc(C(=O)c2ccc(Cl)cc2)cc1] | C₂₆H₂₆ClNO₆ | 483.94 |
| PP73 | 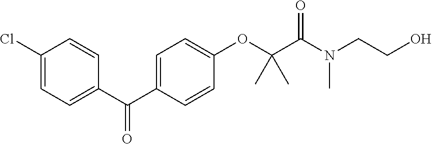 [SMILES: CN(CCO)C(=O)C(C)(C)Oc1ccc(C(=O)c2ccc(Cl)cc2)cc1] | C₂₀H₂₂ClNO₄ | 375.85 |
| PP74 | 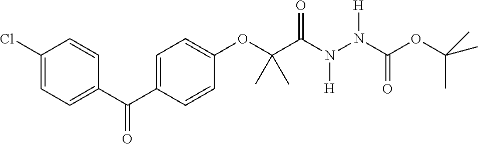 [SMILES: CC(C)(C)OC(=O)NNC(=O)C(C)(C)Oc1ccc(C(=O)c2ccc(Cl)cc2)cc1] | C₂₂H₂₅ClN₂O₅ | 432.90 |
| PP75 | 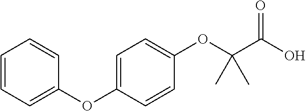 [SMILES: CC(C)(Oc1ccc(Oc2ccccc2)cc1)C(=O)O] | C₁₆H₁₆O₄ | 272.30 |
| PP76 | 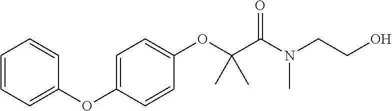 [SMILES: CN(CCO)C(=O)C(C)(C)Oc1ccc(Oc2ccccc2)cc1] | C₁₉H₂₃NO₄ | 329.39 |
| PP77 | 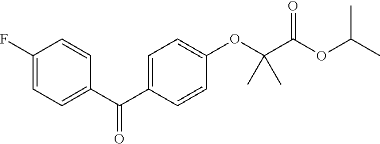 [SMILES: CC(C)OC(=O)C(C)(C)Oc1ccc(C(=O)c2ccc(F)cc2)cc1] | C₂₀H₂₁FO₄ | 344.38 |
| PP78 | 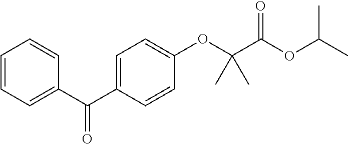 [SMILES: CC(C)OC(=O)C(C)(C)Oc1ccc(C(=O)c2ccccc2)cc1] | C₂₀H₂₂O₄ | 326.39 |
| PP79 | 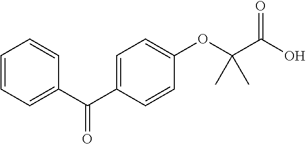 [SMILES: CC(C)(Oc1ccc(C(=O)c2ccccc2)cc1)C(=O)O] | C₁₇H₁₆O₄ | 284.31 |
| PP80 | 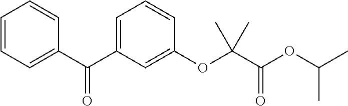 [SMILES: CC(C)OC(=O)C(C)(C)Oc1cccc(C(=O)c2ccccc2)c1] | C₂₀H₂₂O₄ | 326.39 |
| PP81 | 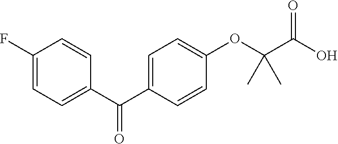 [SMILES: CC(C)(Oc1ccc(C(=O)c2ccc(F)cc2)cc1)C(=O)O] | C₁₇H₁₅FO₄ | 302.30 |
| PP82 | 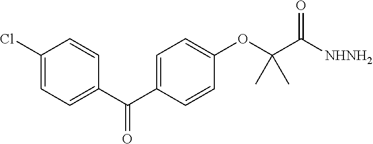 [SMILES: CC(C)(Oc1ccc(C(=O)c2ccc(Cl)cc2)cc1)C(=O)NN] | C₁₇H₁₇ClN₂O₃ | 332.78 |
| PP83 | 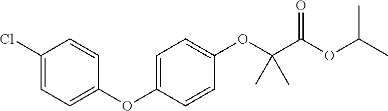 [SMILES: CC(C)OC(=O)C(C)(C)Oc1ccc(Oc2ccc(Cl)cc2)cc1] | C₁₉H₂₁ClO₄ | 348.82 |
| PP84 | 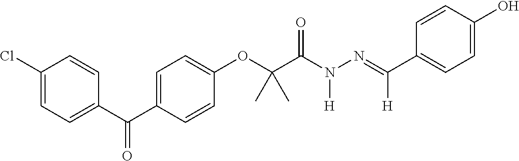 [SMILES: CC(C)(Oc1ccc(C(=O)c2ccc(Cl)cc2)cc1)C(=O)N/N=C/c1ccc(O)cc1] | C₂₄H₂₁ClN₂O₄ | 436.89 |
| PP85 | 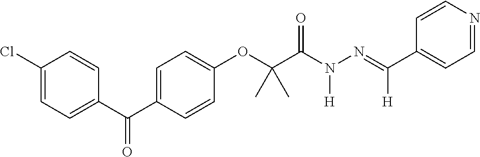 [SMILES: CC(C)(Oc1ccc(C(=O)c2ccc(Cl)cc2)cc1)C(=O)N/N=C/c1ccncc1] | C₂₃H₂₀ClN₃O₃ | 421.88 |
| PP86 | 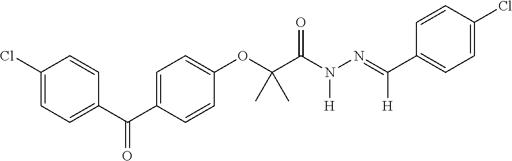 [SMILES: CC(C)(Oc1ccc(C(=O)c2ccc(Cl)cc2)cc1)C(=O)N/N=C/c1ccc(Cl)cc1] | C₂₄H₂₀Cl₂N₂O₃ | 455.33 |
| PP87 | 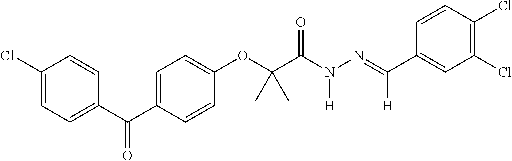 [SMILES: CC(C)(Oc1ccc(C(=O)c2ccc(Cl)cc2)cc1)C(=O)N/N=C/c1ccc(Cl)c(Cl)c1] | C₂₄H₁₉Cl₃N₂O₃ | 489.78 |
| PP88 | 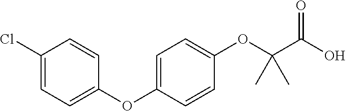 [SMILES: CC(C)(Oc1ccc(Oc2ccc(Cl)cc2)cc1)C(=O)O] | C₁₆H₁₅ClO₄ | 306.74 |
| PP89 | 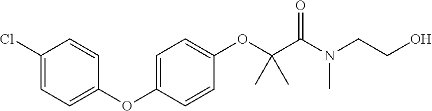 [SMILES: CN(CCO)C(=O)C(C)(C)Oc1ccc(Oc2ccc(Cl)cc2)cc1] | C₁₉H₂₂ClNO₄ | 363.84 |
| PP90 | 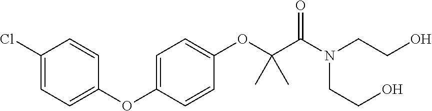 [SMILES: CC(C)(Oc1ccc(Oc2ccc(Cl)cc2)cc1)C(=O)N(CCO)CCO] | C₂₀H₂₄ClNO₅ | 393.86 |
| PP91 | 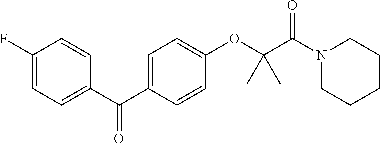 [SMILES: CC(C)(Oc1ccc(C(=O)c2ccc(F)cc2)cc1)C(=O)N1CCCCC1] | C₂₂H₂₄FNO₃ | 369.43 |
| PP92 | 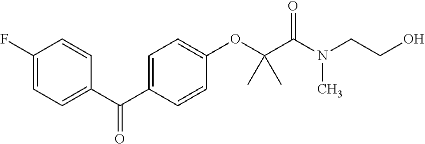 [SMILES: CN(CCO)C(=O)C(C)(C)Oc1ccc(C(=O)c2ccc(F)cc2)cc1] | C₂₀H₂₂FNO₄ | 359.39 |
| PP93 | 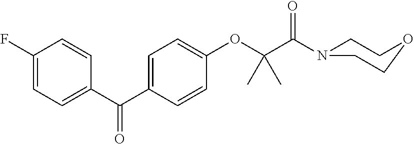 [SMILES: CC(C)(Oc1ccc(C(=O)c2ccc(F)cc2)cc1)C(=O)N1CCOCC1] | C₂₁H₂₂FNO₄ | 371.40 |
| PP94 | 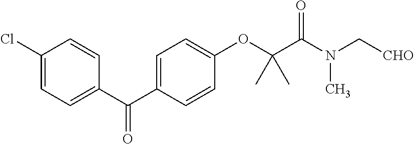 [SMILES: CN(CC=O)C(=O)C(C)(C)Oc1ccc(C(=O)c2ccc(Cl)cc2)cc1] | C₂₀H₂₀ClNO₄ | 373.83 |
| PP95 | 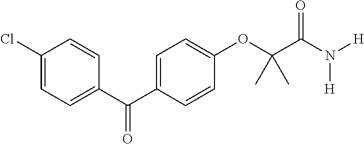 [SMILES: CC(C)(Oc1ccc(C(=O)c2ccc(Cl)cc2)cc1)C(N)=O] | C₁₇H₁₆ClNO₃ | 317.77 |
| PP96 | 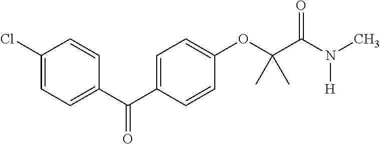 [SMILES: CNC(=O)C(C)(C)Oc1ccc(C(=O)c2ccc(Cl)cc2)cc1] | C₁₈H₁₈ClNO₃ | 331.79 |
| PP97 | 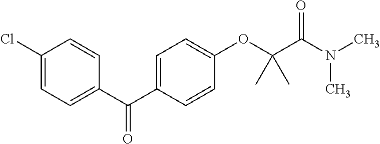 [SMILES: CN(C)C(=O)C(C)(C)Oc1ccc(C(=O)c2ccc(Cl)cc2)cc1] | C₁₉H₂₀ClNO₃ | 345.82 |
| PP98 | 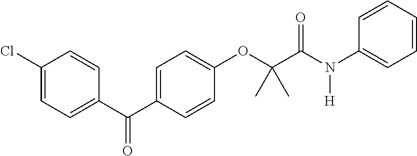 [SMILES: CC(C)(Oc1ccc(C(=O)c2ccc(Cl)cc2)cc1)C(=O)Nc1ccccc1] | C₂₃H₂₀ClNO₃ | 393.86 |
| PP99 | 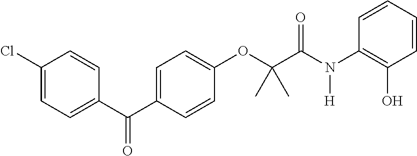 [SMILES: CC(C)(Oc1ccc(C(=O)c2ccc(Cl)cc2)cc1)C(=O)Nc1ccccc1O] | C₂₃H₂₀ClNO₄ | 409.86 |
| PP100 | 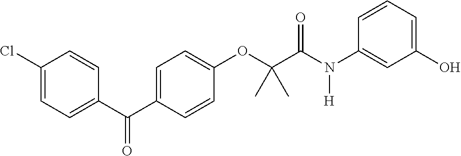 [SMILES: CC(C)(Oc1ccc(C(=O)c2ccc(Cl)cc2)cc1)C(=O)Nc1cccc(O)c1] | C₂₃H₂₀ClNO₄ | 409.86 |
| PP101 | 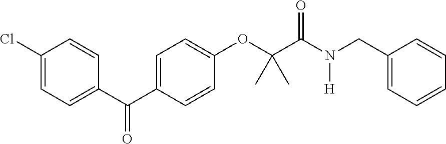 [SMILES: CC(C)(Oc1ccc(C(=O)c2ccc(Cl)cc2)cc1)C(=O)NCc1ccccc1] | C₂₄H₂₂ClNO₃ | 407.89 |
| PP102 | 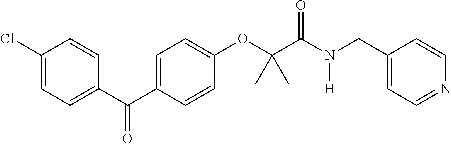 [SMILES: CC(C)(Oc1ccc(C(=O)c2ccc(Cl)cc2)cc1)C(=O)NCc1ccncc1] | C₂₃H₂₁ClN₂O₃ | 408.88 |
| PP103 | 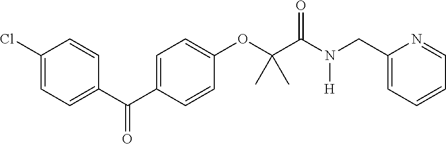 [SMILES: CC(C)(Oc1ccc(C(=O)c2ccc(Cl)cc2)cc1)C(=O)NCc1ccccn1] | C₂₃H₂₁ClN₂O₃ | 408.88 |
| PP104 | 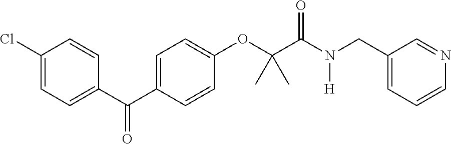 [SMILES: CC(C)(Oc1ccc(C(=O)c2ccc(Cl)cc2)cc1)C(=O)NCc1cccnc1] | C₂₃H₂₁ClN₂O₃ | 408.88 |
| PP105 | 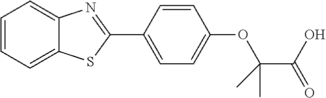 [SMILES: CC(C)(Oc1ccc(-c2nc3ccccc3s2)cc1)C(=O)O] | C₁₇H₁₅NO₃S | 313.37 |
| PP106 | 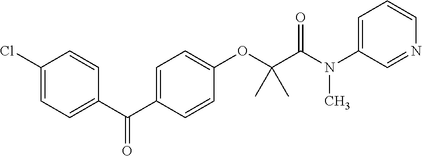 [SMILES: CN(C(=O)C(C)(C)Oc1ccc(C(=O)c2ccc(Cl)cc2)cc1)c1cccnc1] | C₂₃H₂₁ClN₂O₃ | 408.88 |
| PP107 | 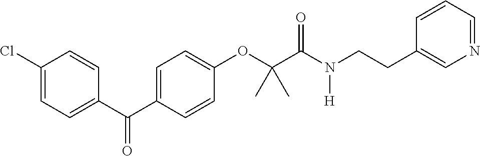 [SMILES: CC(C)(Oc1ccc(C(=O)c2ccc(Cl)cc2)cc1)C(=O)NCCc1cccnc1] | C₂₄H₂₃ClN₂O₃ | 422.90 |
| PP108 | 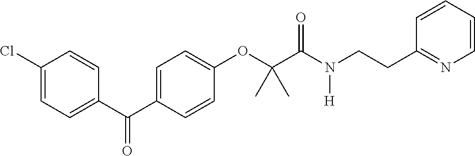 [SMILES: CC(C)(Oc1ccc(C(=O)c2ccc(Cl)cc2)cc1)C(=O)NCCc1ccccn1] | C₂₄H₂₃ClN₂O₃ | 422.90 |
| PP111 | 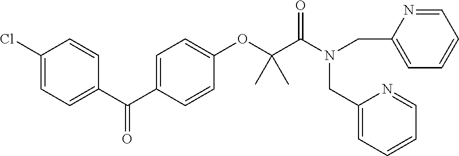 [SMILES: CC(C)(Oc1ccc(C(=O)c2ccc(Cl)cc2)cc1)C(=O)N(Cc1ccccn1)Cc1ccccn1] | C₂₉H₂₆ClN₃O₃ | 499.99 |
| PP112 | 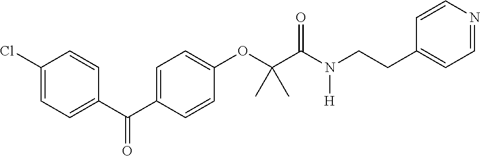 [SMILES: CC(C)(Oc1ccc(C(=O)c2ccc(Cl)cc2)cc1)C(=O)NCCc1ccncc1] | C₂₄H₂₃ClN₂O₃ | 422.90 |
| PP113 | 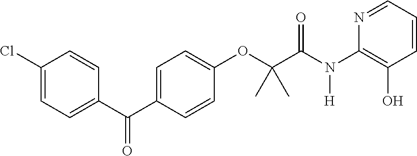 [SMILES: CC(C)(Oc1ccc(C(=O)c2ccc(Cl)cc2)cc1)C(=O)Nc1ncccc1O] | C₂₂H₁₉ClN₂O₄ | 410.85 |
| PP114 | 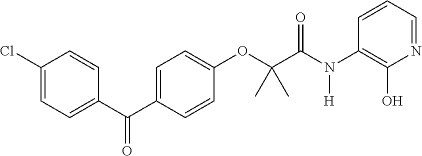 [SMILES: CC(C)(Oc1ccc(C(=O)c2ccc(Cl)cc2)cc1)C(=O)Nc1cccnc1O] | C₂₂H₁₉ClN₂O₄ | 410.85 |
| PP115 | 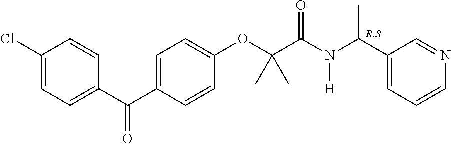 [SMILES: CC(NC(=O)C(C)(C)Oc1ccc(C(=O)c2ccc(Cl)cc2)cc1)c1cccnc1] | C₂₄H₂₃ClN₂O₃ | 422.90 |
| PP116 | 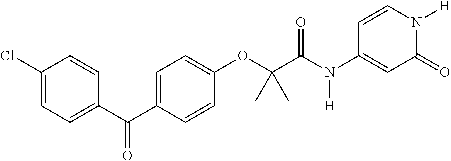 [SMILES: CC(C)(Oc1ccc(C(=O)c2ccc(Cl)cc2)cc1)C(=O)Nc1cc[nH]c(=O)c1] | C₂₂H₁₉ClN₂O₄ | 410.85 |
| PP117 | 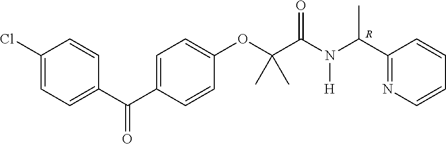 | C₂₄H₂₃ClN₂O₃ | 422.90 |
| PP118 | 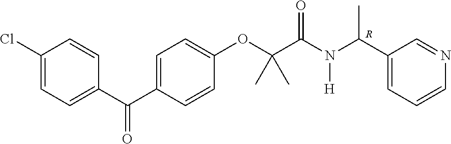 | C₂₄H₂₃ClN₂O₃ | 422.90 |
| PP119 | 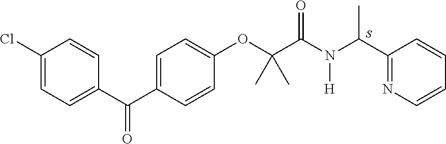 | C₂₄H₂₃ClN₂O₃ | 422.90 |
| PP126 | 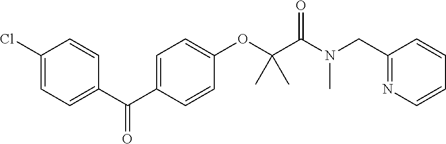 [SMILES: CN(Cc1ccccn1)C(=O)C(C)(C)Oc1ccc(C(=O)c2ccc(Cl)cc2)cc1] | C₂₄H₃₃ClN₂O₃ | 422.90 |
| PP127 | 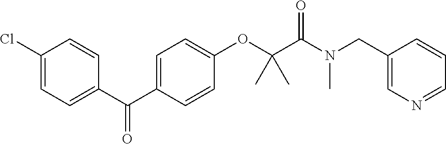 [SMILES: CN(Cc1cccnc1)C(=O)C(C)(C)Oc1ccc(C(=O)c2ccc(Cl)cc2)cc1] | C₂₄H₂₃ClN₂O₃ | 422.90 |
| PP128 | 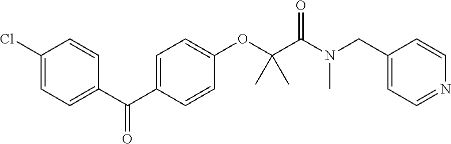 [SMILES: CN(Cc1ccncc1)C(=O)C(C)(C)Oc1ccc(C(=O)c2ccc(Cl)cc2)cc1] | C₂₄H₂₃ClN₂O₃ | 422.90 |
| PP129 | 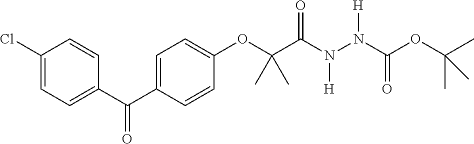 [SMILES: CC(C)(C)OC(=O)NNC(=O)C(C)(C)Oc1ccc(C(=O)c2ccc(Cl)cc2)cc1] | C₂₂H₂₅ClN₂O₅ | 432.90 |
| PP130 | 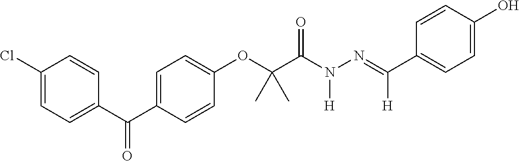 [SMILES: CC(C)(Oc1ccc(C(=O)c2ccc(Cl)cc2)cc1)C(=O)N/N=C/c1ccc(O)cc1] | C₂₄H₂₁ClN₂O₄ | 436.89 |
| PP131 | 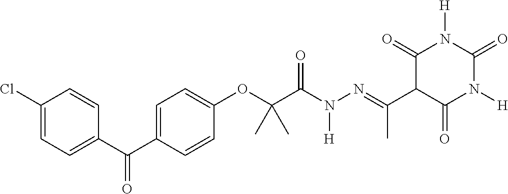 [SMILES: C/C(=N\NC(=O)C(C)(C)Oc1ccc(C(=O)c2ccc(Cl)cc2)cc1)C1C(=O)NC(=O)NC1=O] | C₂₃H₂₁ClN₄O₆ | 484.89 |
| PP132 | 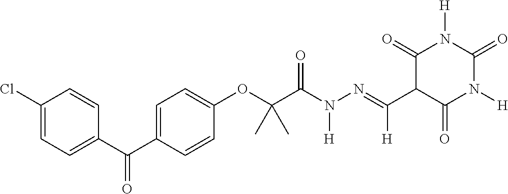 [SMILES: CC(C)(Oc1ccc(C(=O)c2ccc(Cl)cc2)cc1)C(=O)N/N=C/C1C(=O)NC(=O)NC1=O] | C₂₂H₁₉ClN₄O₆ | 470.86 |
| PP133 | 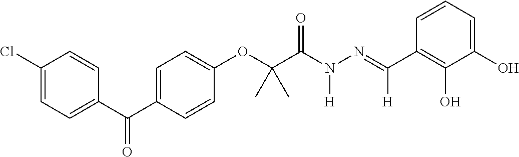 [SMILES: CC(C)(Oc1ccc(C(=O)c2ccc(Cl)cc2)cc1)C(=O)N/N=C/c1cccc(O)c1O] | C₂₄H₂₁ClN₂O₅ | 452.89 |
| PP134 | 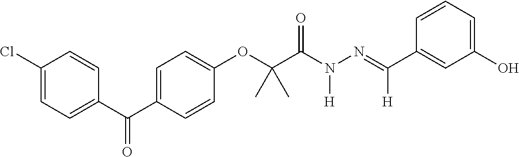 [SMILES: CC(C)(Oc1ccc(C(=O)c2ccc(Cl)cc2)cc1)C(=O)N/N=C/c1cccc(O)c1] | C₂₄H₂₁ClN₂O₄ | 436.89 |
| PP135 | 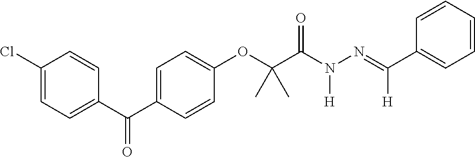 [SMILES: CC(C)(Oc1ccc(C(=O)c2ccc(Cl)cc2)cc1)C(=O)N/N=C/c1ccccc1] | C₂₄H₂₁ClN₂O₃ | 420.89 |
| PP136 | 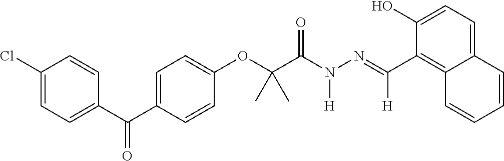 [SMILES: CC(C)(Oc1ccc(C(=O)c2ccc(Cl)cc2)cc1)C(=O)N/N=C/c1c(O)ccc2ccccc12] | C₂₈H₂₃ClN₂O₄ | 486.95 |
| PP137 | 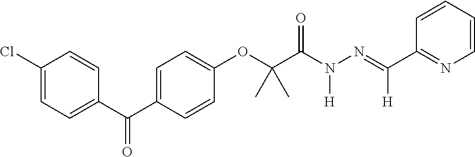 [SMILES: CC(C)(Oc1ccc(C(=O)c2ccc(Cl)cc2)cc1)C(=O)N/N=C/c1ccccn1] | C₂₃H₂₀ClN₃O₃ | 421.88 |
| PP138 | 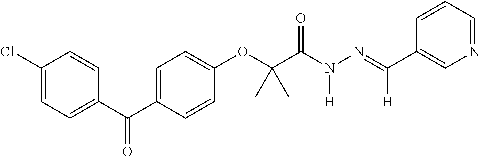 [SMILES: CC(C)(Oc1ccc(C(=O)c2ccc(Cl)cc2)cc1)C(=O)N/N=C/c1cccnc1] | C₂₃H₂₀ClN₃O₃ | 421.88 |
| PP139 | 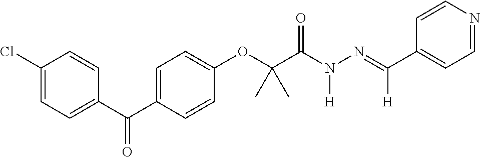 [SMILES: CC(C)(Oc1ccc(C(=O)c2ccc(Cl)cc2)cc1)C(=O)N/N=C/c1ccncc1] | C₂₃H₂₀ClN₃O₃ | 421.88 |
| PP140 | 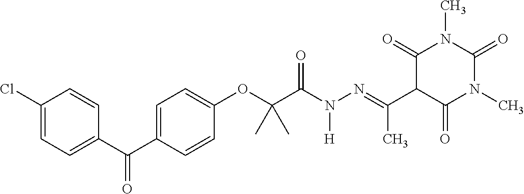 [SMILES: C/C(=N\NC(=O)C(C)(C)Oc1ccc(C(=O)c2ccc(Cl)cc2)cc1)C1C(=O)N(C)C(=O)N(C)C1=O] | C₂₅H₂₅ClN₄O₆ | 512.94 |
| PP141 | 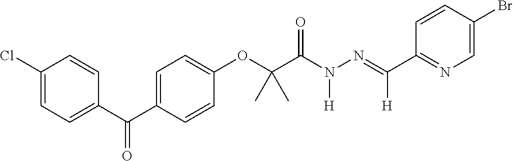 [SMILES: CC(C)(Oc1ccc(C(=O)c2ccc(Cl)cc2)cc1)C(=O)N/N=C/c1ccc(Br)cn1] | C₂₃H₁₉BrClN₃O₃ | 500.77 |
| PP142 | 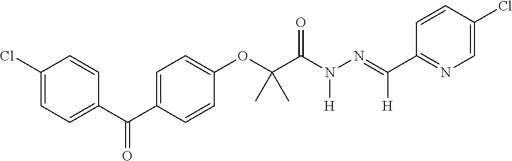 [SMILES: CC(C)(Oc1ccc(C(=O)c2ccc(Cl)cc2)cc1)C(=O)N/N=C/c1ccc(Cl)cn1] | C₂₃H₁₉Cl₂N₃O₃ | 456.32 |
| PP143 | 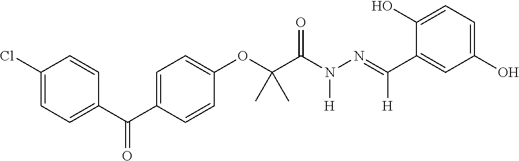 [SMILES: CC(C)(Oc1ccc(C(=O)c2ccc(Cl)cc2)cc1)C(=O)N/N=C/c1cc(O)ccc1O] | C₂₄H₂₁ClN₂O₅ | 452.89 |
| PP144 | 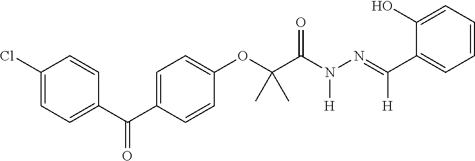 [SMILES: CC(C)(Oc1ccc(C(=O)c2ccc(Cl)cc2)cc1)C(=O)N/N=C/c1ccccc1O] | C₂₄H₂₁ClN₂O₄ | 436.89 |
| PP145 | 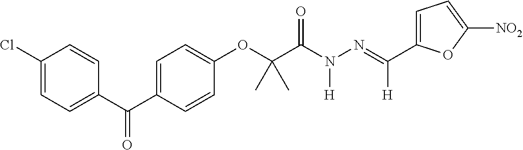 [SMILES: CC(C)(Oc1ccc(C(=O)c2ccc(Cl)cc2)cc1)C(=O)N/N=C/c1ccc([N+](=O)[O-])o1] | C₂₂H₁₈ClN₃O₆ | 455.85 |
| PP146 | 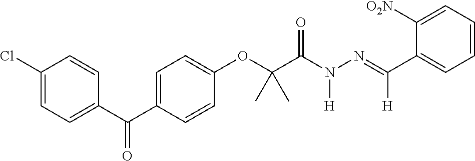 [SMILES: CC(C)(Oc1ccc(C(=O)c2ccc(Cl)cc2)cc1)C(=O)N/N=C/c1ccccc1[N+](=O)[O-]] | C₂₄H₂₀ClN₃O₅ | 465.89 |
| PP147 | 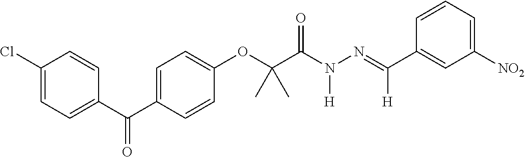 [SMILES: CC(C)(Oc1ccc(C(=O)c2ccc(Cl)cc2)cc1)C(=O)N/N=C/c1cccc([N+](=O)[O-])c1] | C₂₄H₂₀ClN₃O₅ | 465.89 |
| PP148 | 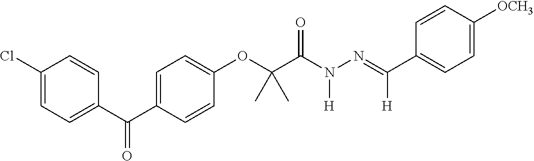 [SMILES: COc1ccc(/C=N/NC(=O)C(C)(C)Oc2ccc(C(=O)c3ccc(Cl)cc3)cc2)cc1] | C₂₅H₂₃ClN₂O₄ | 450.91 |
| PP149 | 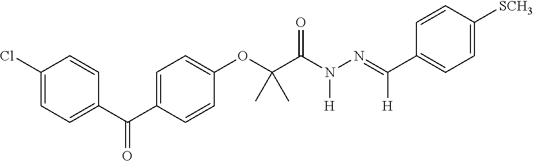 [SMILES: CSc1ccc(/C=N/NC(=O)C(C)(C)Oc2ccc(C(=O)c3ccc(Cl)cc3)cc2)cc1] | C₂₅H₂₃ClN₂O₃S | 466.98 |
| PP150 | 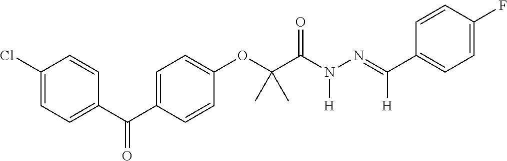 [SMILES: CC(C)(Oc1ccc(C(=O)c2ccc(Cl)cc2)cc1)C(=O)N/N=C/c1ccc(F)cc1] | C₂₄H₂₀ClFN₂O₃ | 438.88 |
| PP151 | 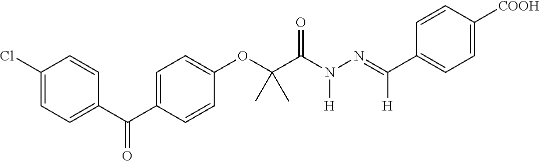 [SMILES: CC(C)(Oc1ccc(C(=O)c2ccc(Cl)cc2)cc1)C(=O)N/N=C/c1ccc(C(=O)O)cc1] | C₂₅H₂₁ClN₂O₅ | 464.90 |
| PP152 | 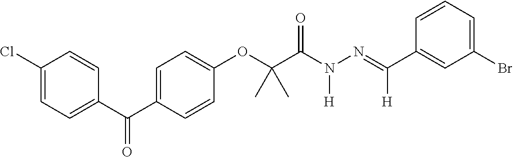 [SMILES: CC(C)(Oc1ccc(C(=O)c2ccc(Cl)cc2)cc1)C(=O)N/N=C/c1cccc(Br)c1] | C₂₄H₂₀BrClN₂O₃ | 499.78 |
| PP153 | 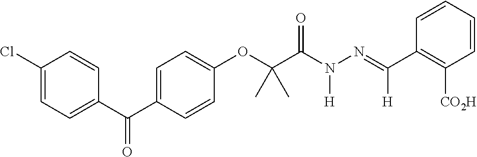 [SMILES: CC(C)(Oc1ccc(C(=O)c2ccc(Cl)cc2)cc1)C(=O)N/N=C/c1ccccc1C(=O)O] | C₂₅H₂₁ClN₂O₅ | 464.90 |
| PP154 | 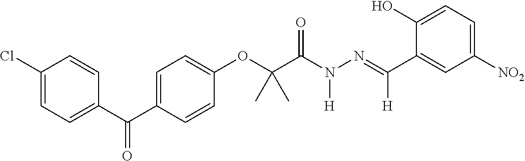 [SMILES: CC(C)(Oc1ccc(C(=O)c2ccc(Cl)cc2)cc1)C(=O)N/N=C/c1cc([N+](=O)[O-])ccc1O] | C₂₄H₂₀ClN₃O₆ | 481.89 |
| PP155 | 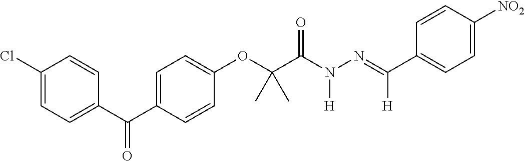 [SMILES: CC(C)(Oc1ccc(C(=O)c2ccc(Cl)cc2)cc1)C(=O)N/N=C/c1ccc([N+](=O)[O-])cc1] | C₂₄H₂₀ClN₃O₅ | 465.89 |
| PP156 | 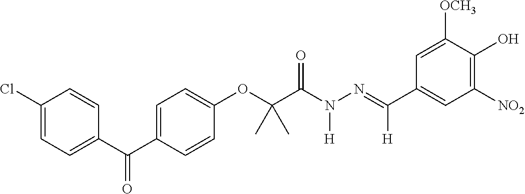 [SMILES: COc1cc(/C=N/NC(=O)C(C)(C)Oc2ccc(C(=O)c3ccc(Cl)cc3)cc2)cc([N+](=O)[O-])c1O] | C₂₅H₂₂ClN₃O₇ | 511.91 |
| PP157 | 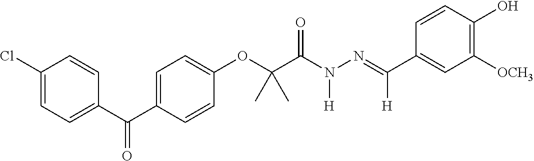 [SMILES: COc1cc(/C=N/NC(=O)C(C)(C)Oc2ccc(C(=O)c3ccc(Cl)cc3)cc2)ccc1O] | C₂₅H₂₃ClN₂O₅ | 466.91 |
| PP158 | 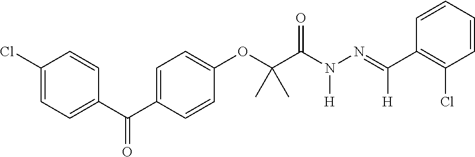 [SMILES: CC(C)(Oc1ccc(C(=O)c2ccc(Cl)cc2)cc1)C(=O)N/N=C/c1ccccc1Cl] | C₂₄H₂₀Cl₂N₂O₃ | 455.33 |
| PP159 | 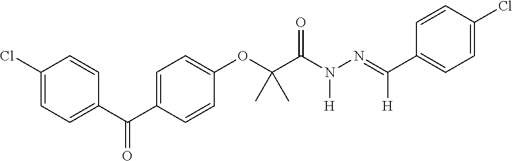 [SMILES: CC(C)(Oc1ccc(C(=O)c2ccc(Cl)cc2)cc1)C(=O)N/N=C/c1ccc(Cl)cc1] | C₂₄H₂₀Cl₂N₂O₃ | 455.33 |
| PP160 | 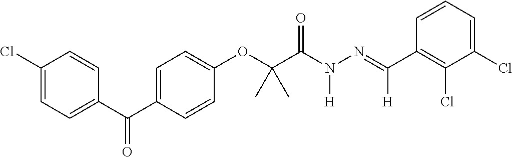 [SMILES: CC(C)(Oc1ccc(C(=O)c2ccc(Cl)cc2)cc1)C(=O)N/N=C/c1cccc(Cl)c1Cl] | C₂₄H₁₉Cl₃N₂O₃ | 489.78 |
| PP161 | 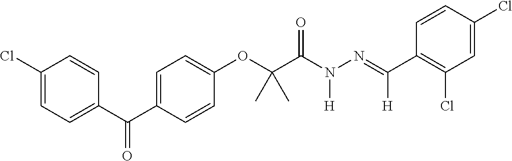 [SMILES: CC(C)(Oc1ccc(C(=O)c2ccc(Cl)cc2)cc1)C(=O)N/N=C/c1ccc(Cl)cc1Cl] | C₂₄H₁₉Cl₃N₂O₃ | 489.78 |
| PP162 | 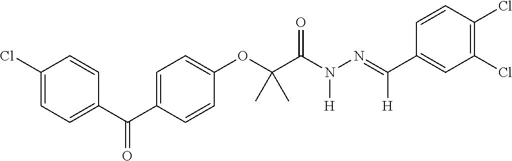 [SMILES: CC(C)(Oc1ccc(C(=O)c2ccc(Cl)cc2)cc1)C(=O)N/N=C/c1ccc(Cl)c(Cl)c1] | C₂₄H₁₉Cl₃N₂O₃ | 489.78 |
| PP163 | 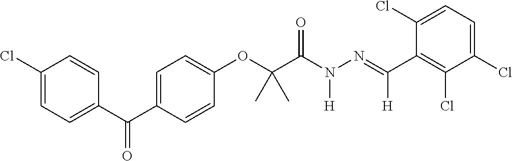 [SMILES: CC(C)(Oc1ccc(C(=O)c2ccc(Cl)cc2)cc1)C(=O)N/N=C/c1c(Cl)ccc(Cl)c1Cl] | C₂₄H₁₈Cl₄N₂O₃ | 524.22 |
| PP164 | 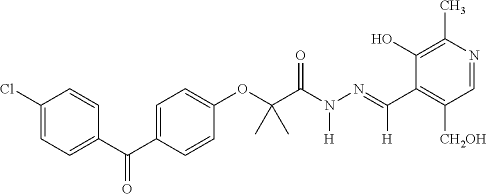 [SMILES: Cc1ncc(CO)c(/C=N/NC(=O)C(C)(C)Oc2ccc(C(=O)c3ccc(Cl)cc3)cc2)c1O] | C₂₅H₂₄ClN₃O₅ | 481.93 |
| PP165 | 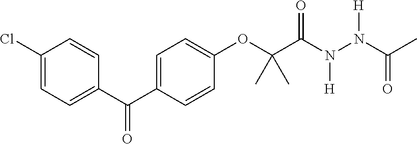 [SMILES: CC(=O)NNC(=O)C(C)(C)Oc1ccc(C(=O)c2ccc(Cl)cc2)cc1] | C₁₉H₁₉ClN₂O₄ | 374.82 |
| PP166 | 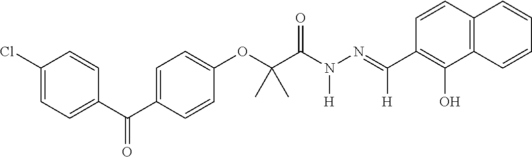 [SMILES: CC(C)(Oc1ccc(C(=O)c2ccc(Cl)cc2)cc1)C(=O)N/N=C/c1ccc2ccccc2c1O] | C₂₈H₂₃ClN₂O₄ | 486.95 |
| PP167 | 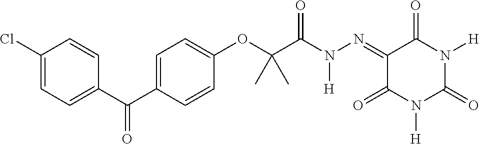 [SMILES: CC(C)(Oc1ccc(C(=O)c2ccc(Cl)cc2)cc1)C(=O)NN=C1C(=O)NC(=O)NC1=O] | C₂₁H₁₇ClN₄O₆ | 456.84 |
| PP168 | 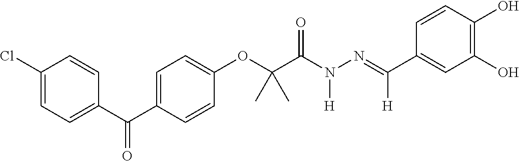 [SMILES: CC(C)(Oc1ccc(C(=O)c2ccc(Cl)cc2)cc1)C(=O)N/N=C/c1ccc(O)c(O)c1] | C₂₄H₂₁ClN₂O₅ | 452.89 |
| PP169 | 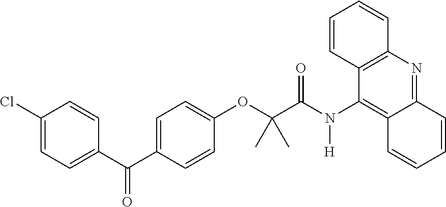 [SMILES: CC(C)(Oc1ccc(C(=O)c2ccc(Cl)cc2)cc1)C(=O)Nc1c2ccccc2nc2ccccc12] | C₃₀H₂₃ClN₂O₃ | 494.97 |
| PP170 | 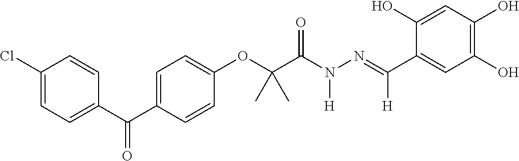 [SMILES: CC(C)(Oc1ccc(C(=O)c2ccc(Cl)cc2)cc1)C(=O)N/N=C/c1cc(O)c(O)cc1O] | C₂₄H₂₁ClN₂O₆ | 468.89 |
| PP171 | 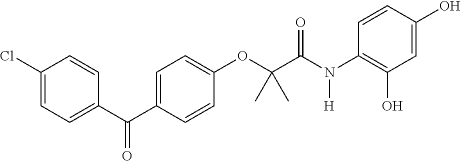 [SMILES: CC(C)(Oc1ccc(C(=O)c2ccc(Cl)cc2)cc1)C(=O)Nc1ccc(O)cc1O] | C₂₃H₂₀ClNO₅ | 425.86 |
| PP172 | 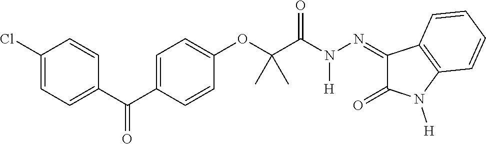 [SMILES: CC(C)(Oc1ccc(C(=O)c2ccc(Cl)cc2)cc1)C(=O)N/N=C1\C(=O)Nc2ccccc21] | C₂₅H₂₀ClN₃O₄ | 461.90 |
| PP173 | 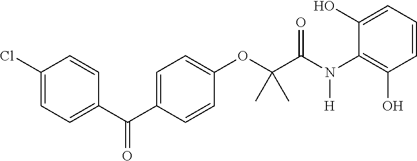 [SMILES: CC(C)(Oc1ccc(C(=O)c2ccc(Cl)cc2)cc1)C(=O)Nc1c(O)cccc1O] | C₂₃H₂₀ClNO₅ | 425.86 |
| PP174 | 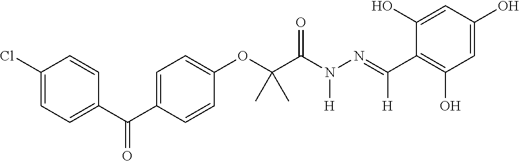 [SMILES: CC(C)(Oc1ccc(C(=O)c2ccc(Cl)cc2)cc1)C(=O)N/N=C/c1c(O)cc(O)cc1O] | C₂₄H₂₁ClN₂O₆ | 468.89 |
| PP175 | 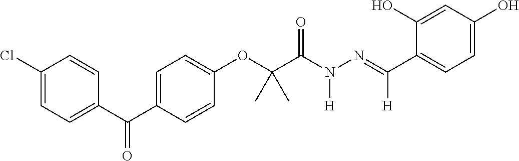 [SMILES: CC(C)(Oc1ccc(C(=O)c2ccc(Cl)cc2)cc1)C(=O)N/N=C/c1ccc(O)cc1O] | C₂₃H₂₀ClNO₅ | 425.86 |
| PP176 | 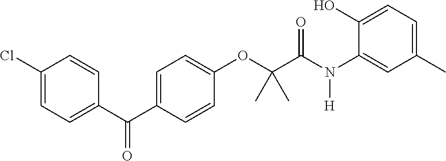 [SMILES: Cc1ccc(O)c(NC(=O)C(C)(C)Oc2ccc(C(=O)c3ccc(Cl)cc3)cc2)c1] | C₂₄H₂₂ClNO₄ | 423.89 |
| PP177 | 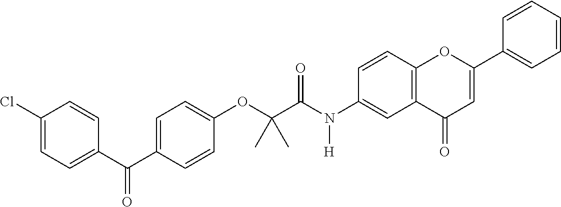 [SMILES: CC(C)(Oc1ccc(C(=O)c2ccc(Cl)cc2)cc1)C(=O)Nc1ccc2oc(-c3ccccc3)cc(=O)c2c1] | C₃₂H₂₄ClNO₅ | 537.99 |
| PP178 | 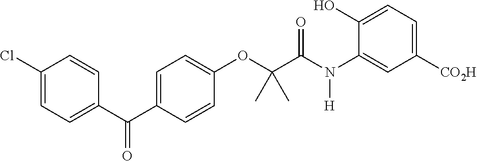 [SMILES: CC(C)(Oc1ccc(C(=O)c2ccc(Cl)cc2)cc1)C(=O)Nc1cc(C(=O)O)ccc1O] | C₂₄H₂₀ClNO₆ | 453.87 |
| PP179 | 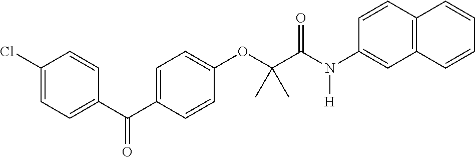 [SMILES: CC(C)(Oc1ccc(C(=O)c2ccc(Cl)cc2)cc1)C(=O)Nc1ccc2ccccc2c1] | C₂₇H₂₂ClNO₃ | 443.92 |
| PP180 | 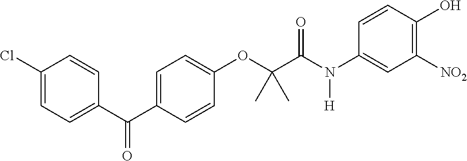 [SMILES: CC(C)(Oc1ccc(C(=O)c2ccc(Cl)cc2)cc1)C(=O)Nc1ccc(O)c([N+](=O)[O-])c1] | C₂₃H₁₉ClN₂O₆ | 454.86 |
| PP181 | 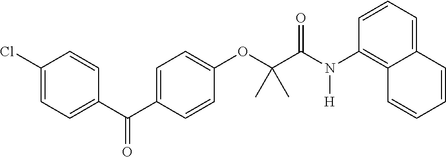 [SMILES: CC(C)(Oc1ccc(C(=O)c2ccc(Cl)cc2)cc1)C(=O)Nc1cccc2ccccc12] | C₂₇H₂₂ClNO₃ | 443.92 |
| PP182 | 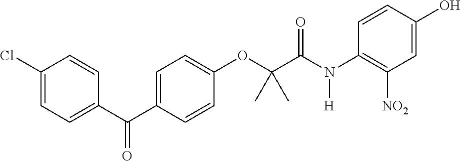 [SMILES: CC(C)(Oc1ccc(C(=O)c2ccc(Cl)cc2)cc1)C(=O)Nc1ccc(O)cc1[N+](=O)[O-]] | C₂₃H₁₉ClN₂O₆ | 454.86 |
| PP183 | 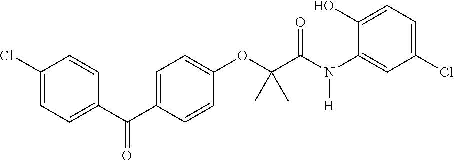 [SMILES: CC(C)(Oc1ccc(C(=O)c2ccc(Cl)cc2)cc1)C(=O)Nc1cc(Cl)ccc1O] | C₂₃H₁₉Cl₂NO₄ | 444.31 |
| PP184 | 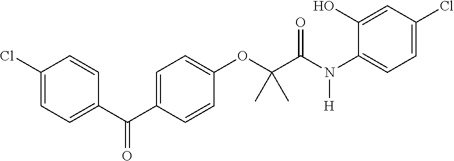 [SMILES: CC(C)(Oc1ccc(C(=O)c2ccc(Cl)cc2)cc1)C(=O)Nc1ccc(Cl)cc1O] | C₂₃H₁₉Cl₂NO₄ | 444.31 |
| PP185 | 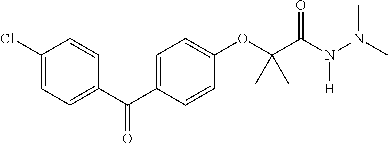 [SMILES: CN(C)NC(=O)C(C)(C)Oc1ccc(C(=O)c2ccc(Cl)cc2)cc1] | C₁₉H₂₁ClN₂O₃ | 360.83 |
| PP186 | 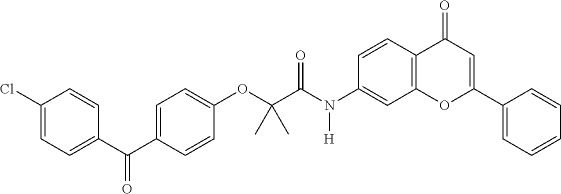 [SMILES: CC(C)(Oc1ccc(C(=O)c2ccc(Cl)cc2)cc1)C(=O)Nc1ccc2c(=O)cc(-c3ccccc3)oc2c1] | C₃₂H₂₄ClNO₅ | 537.99 |
| PP187 | 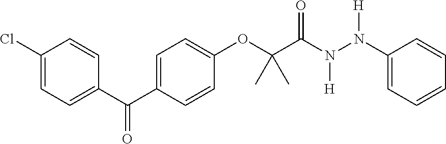 [SMILES: CC(C)(Oc1ccc(C(=O)c2ccc(Cl)cc2)cc1)C(=O)NNc1ccccc1] | C₂₃H₂₁ClN₂O₃ | 408.88 |
| PP188 | 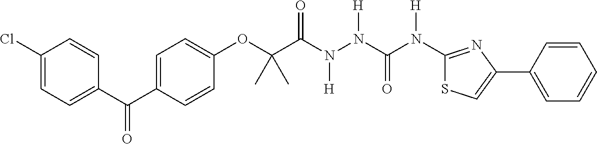 [SMILES: CC(C)(Oc1ccc(C(=O)c2ccc(Cl)cc2)cc1)C(=O)NNC(=O)Nc1nc(-c2ccccc2)cs1] | C₂₇H₂₃ClN₄O₄S | 535.01 |
| PP189 | 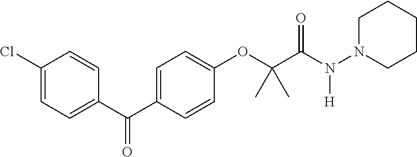 [SMILES: CC(C)(Oc1ccc(C(=O)c2ccc(Cl)cc2)cc1)C(=O)NN1CCCCC1] | C₂₂H₂₅ClN₂O₃ | 400.90 |
| PP190 | 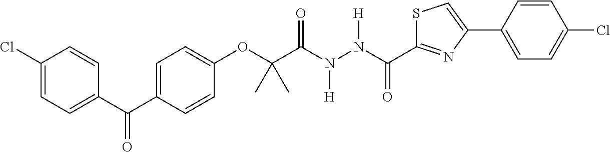 [SMILES: CC(C)(Oc1ccc(C(=O)c2ccc(Cl)cc2)cc1)C(=O)NNC(=O)c1nc(-c2ccc(Cl)cc2)cs1] | C₂₇H₂₁Cl₂N₃O₄S | 554.44 |
| PP191 | 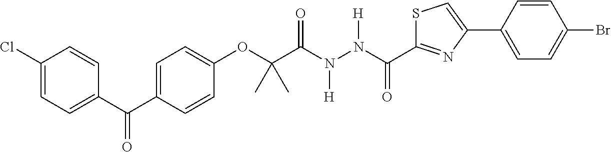 [SMILES: CC(C)(Oc1ccc(C(=O)c2ccc(Cl)cc2)cc1)C(=O)NNC(=O)c1nc(-c2ccc(Br)cc2)cs1] | C₂₇H₂₁BrClN₃O₄S | 598.90 |
| PP192 | 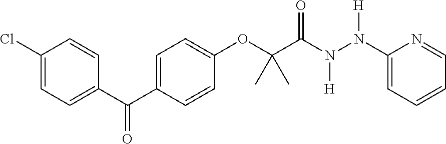 [SMILES: CC(C)(Oc1ccc(C(=O)c2ccc(Cl)cc2)cc1)C(=O)NNc1ccccn1] | C₂₂H₂₀ClN₃O₃ | 409.87 |
| PP193 | 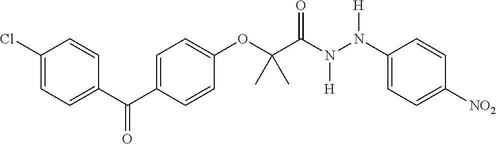 [SMILES: CC(C)(Oc1ccc(C(=O)c2ccc(Cl)cc2)cc1)C(=O)NNc1ccc([N+](=O)[O-])cc1] | C₂₃H₂₀ClN₃O₅ | 453.88 |
| PP194 | 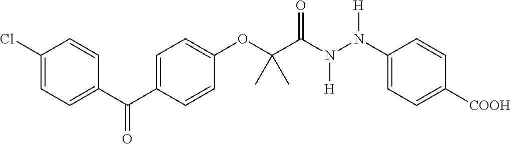 [SMILES: CC(C)(Oc1ccc(C(=O)c2ccc(Cl)cc2)cc1)C(=O)NNc1ccc(C(=O)O)cc1] | C₂₄H₂₁ClN₂O₅ | 452.89 |
| PP195 | 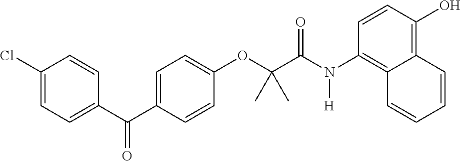 [SMILES: CC(C)(Oc1ccc(C(=O)c2ccc(Cl)cc2)cc1)C(=O)Nc1ccc(O)c2ccccc12] | C₂₇H₂₂ClNO₄ | 459.92 |
| PP196 | 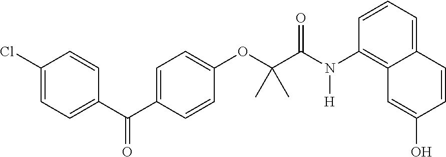 [SMILES: CC(C)(Oc1ccc(C(=O)c2ccc(Cl)cc2)cc1)C(=O)Nc1cccc2ccc(O)cc12] | C₂₇H₂₂ClNO₄ | 459.92 |
| PP197 | 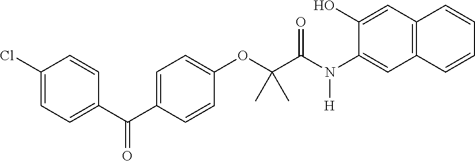 [SMILES: CC(C)(Oc1ccc(C(=O)c2ccc(Cl)cc2)cc1)C(=O)Nc1cc2ccccc2cc1O] | C₂₇H₂₂ClNO₄ | 459.92 |
| PP198 | 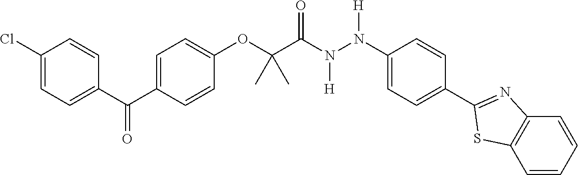 [SMILES: CC(C)(Oc1ccc(C(=O)c2ccc(Cl)cc2)cc1)C(=O)NNc1ccc(-c2nc3ccccc3s2)cc1] | C₃₀H₂₄ClN₃O₃S | 542.05 |
| PP199 | 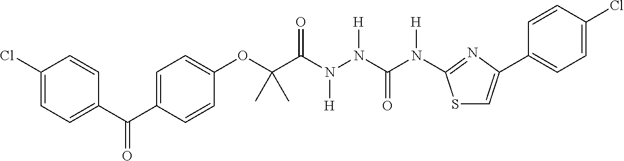 [SMILES: CC(C)(Oc1ccc(C(=O)c2ccc(Cl)cc2)cc1)C(=O)NNC(=O)Nc1nc(-c2ccc(Cl)cc2)cs1] | C₂₇H₂₂Cl₂N₄O₄S | 569.46 |
| PP200 | 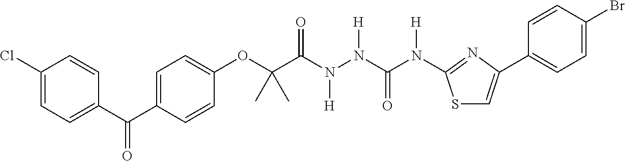 [SMILES: CC(C)(Oc1ccc(C(=O)c2ccc(Cl)cc2)cc1)C(=O)NNC(=O)Nc1nc(-c2ccc(Br)cc2)cs1] | C₂₇H₂₂BrClN₄O₄S | 613.91 |
| PP201 | 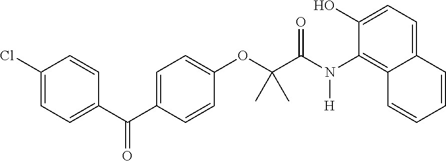 [SMILES: CC(C)(Oc1ccc(C(=O)c2ccc(Cl)cc2)cc1)C(=O)Nc1c(O)ccc2ccccc12] | C₂₇H₂₂ClNO₄ | 459.92 |
| PP202 | 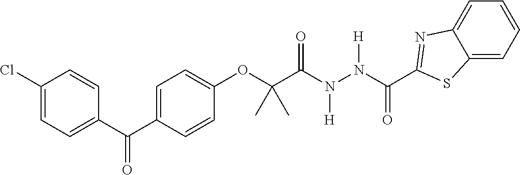 [SMILES: CC(C)(Oc1ccc(C(=O)c2ccc(Cl)cc2)cc1)C(=O)NNC(=O)c1nc2ccccc2s1] | C₂₅H₂₀ClN₃O₄S | 493.96 |
| PP203 | 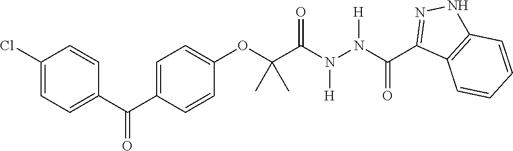 [SMILES: CC(C)(Oc1ccc(C(=O)c2ccc(Cl)cc2)cc1)C(=O)NNC(=O)c1n[nH]c2ccccc12] | C₂₅H₂₁ClN₄O₄ | 476.91 |
| PP204 | 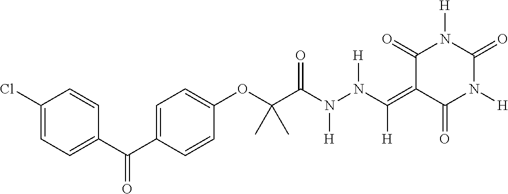 [SMILES: CC(C)(Oc1ccc(C(=O)c2ccc(Cl)cc2)cc1)C(=O)NNC=C1C(=O)NC(=O)NC1=O] | C₂₂H₁₉ClN₄O₆ | 470.86 |
| PP205 | 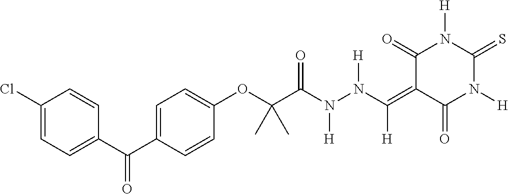 [SMILES: CC(C)(Oc1ccc(C(=O)c2ccc(Cl)cc2)cc1)C(=O)NNC=C1C(=O)NC(=S)NC1=O] | C₂₂H₁₉ClN₄O₅S | 486.93 |
| PP206 | 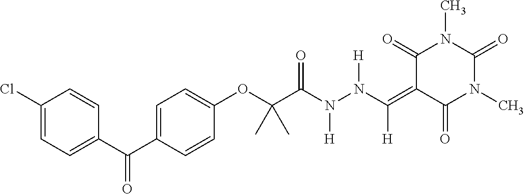 [SMILES: CN1C(=O)C(=CNNC(=O)C(C)(C)Oc2ccc(C(=O)c3ccc(Cl)cc3)cc2)C(=O)N(C)C1=O] | C₂₄H₂₃ClN₄O₆ | 498.92 |
| PP215 | 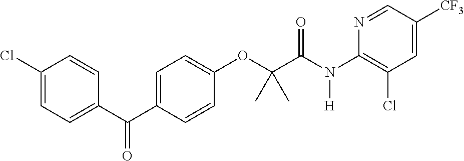 [SMILES: CC(C)(Oc1ccc(C(=O)c2ccc(Cl)cc2)cc1)C(=O)Nc1ncc(C(F)(F)F)cc1Cl] | C₂₃H₁₇Cl₂F₃N₂O₃ | 497.29 |
| PP218 | 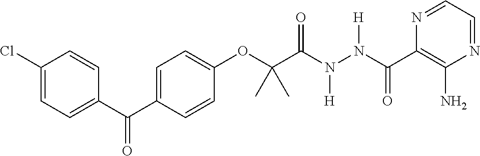 [SMILES: CC(C)(Oc1ccc(C(=O)c2ccc(Cl)cc2)cc1)C(=O)NNC(=O)c1nccnc1N] | C₂₂H₂₀ClN₅O₄ | 453.88 |
| PP219 | 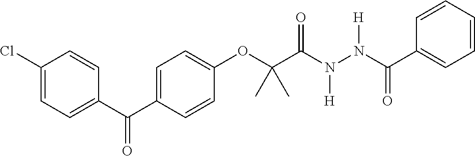 [SMILES: CC(C)(Oc1ccc(C(=O)c2ccc(Cl)cc2)cc1)C(=O)NNC(=O)c1ccccc1] | C₂₄H₂₁ClN₂O₄ | 436.89 |
| PP220 | 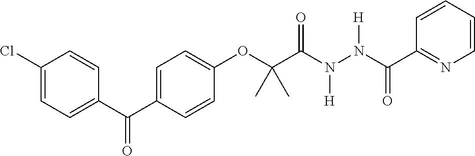 [SMILES: CC(C)(Oc1ccc(C(=O)c2ccc(Cl)cc2)cc1)C(=O)NNC(=O)c1ccccn1] | C₂₃H₂₀ClN₃O₄ | 437.88 |
| PP221 | 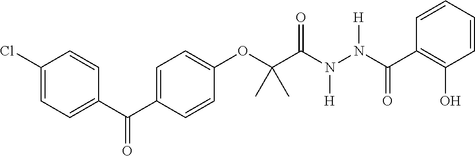 [SMILES: CC(C)(Oc1ccc(C(=O)c2ccc(Cl)cc2)cc1)C(=O)NNC(=O)c1ccccc1O] | C₂₄H₂₁ClN₂O₅ | 452.89 |
| PP222 | 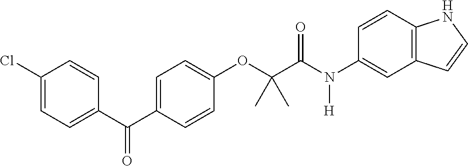 [SMILES: CC(C)(Oc1ccc(C(=O)c2ccc(Cl)cc2)cc1)C(=O)Nc1ccc2[nH]ccc2c1] | C₂₅H₂₁ClN₂O₃ | 432.90 |
| PP223 | 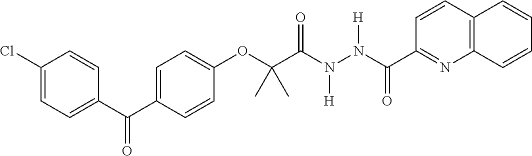 [SMILES: CC(C)(Oc1ccc(C(=O)c2ccc(Cl)cc2)cc1)C(=O)NNC(=O)c1ccc2ccccc2n1] | C₂₇H₂₂ClN₃O₄ | 487.93 |
| PP224 | 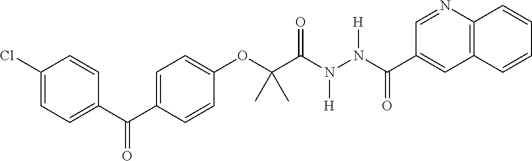 [SMILES: CC(C)(Oc1ccc(C(=O)c2ccc(Cl)cc2)cc1)C(=O)NNC(=O)c1cnc2ccccc2c1] | C₂₇H₂₂ClN₃O₄ | 487.93 |
| PP225 | 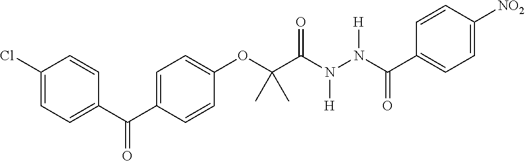 [SMILES: CC(C)(Oc1ccc(C(=O)c2ccc(Cl)cc2)cc1)C(=O)NNC(=O)c1ccc([N+](=O)[O-])cc1] | C₂₄H₂₀ClN₃O₆ | 481.89 |
| PP226 | 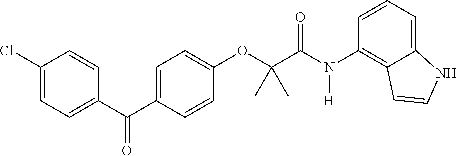 [SMILES: CC(C)(Oc1ccc(C(=O)c2ccc(Cl)cc2)cc1)C(=O)Nc1cccc2[nH]ccc12] | C₂₅H₂₁ClN₂O₃ | 432.90 |
| PP227 | 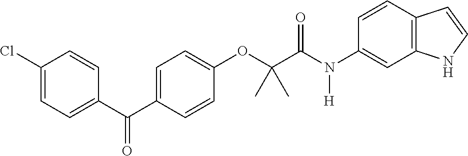 [SMILES: CC(C)(Oc1ccc(C(=O)c2ccc(Cl)cc2)cc1)C(=O)Nc1ccc2cc[nH]c2c1] | C₂₅H₂₁ClN₂O₃ | 432.90 |
| PP228 | 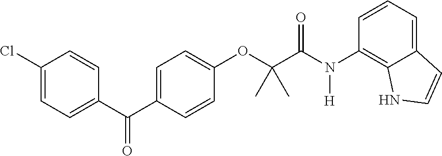 [SMILES: CC(C)(Oc1ccc(C(=O)c2ccc(Cl)cc2)cc1)C(=O)Nc1cccc2cc[nH]c12] | C₂₅H₂₁ClN₂O₃ | 432.90 |
| PP229 | 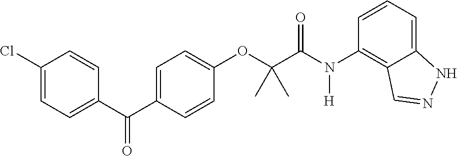 [SMILES: CC(C)(Oc1ccc(C(=O)c2ccc(Cl)cc2)cc1)C(=O)Nc1cccc2[nH]ncc12] | C₂₄H₂₀ClN₃O₃ | 433.89 |
| PP230 | 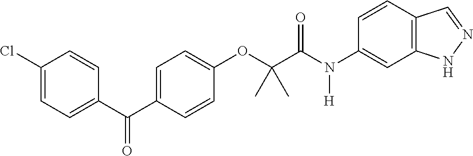 [SMILES: CC(C)(Oc1ccc(C(=O)c2ccc(Cl)cc2)cc1)C(=O)Nc1ccc2cn[nH]c2c1] | C₂₄H₂₀ClN₃O₃ | 433.89 |
| PP231 | 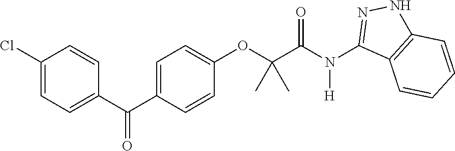 [SMILES: CC(C)(Oc1ccc(C(=O)c2ccc(Cl)cc2)cc1)C(=O)Nc1n[nH]c2ccccc12] | C₂₄H₂₀ClN₃O₃ | 433.89 |
| PP232 | 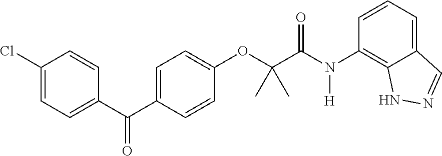 [SMILES: CC(C)(Oc1ccc(C(=O)c2ccc(Cl)cc2)cc1)C(=O)Nc1cccc2cn[nH]c12] | C₂₄H₂₀ClN₃O₃ | 433.89 |
| PP233 | 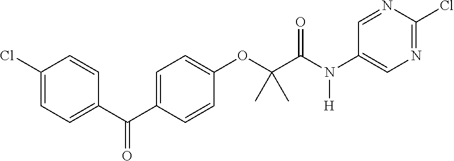 [SMILES: CC(C)(Oc1ccc(C(=O)c2ccc(Cl)cc2)cc1)C(=O)Nc1cnc(Cl)nc1] | C₂₁H₁₇Cl₂N₃O₃ | 430.28 |
| PP234 | 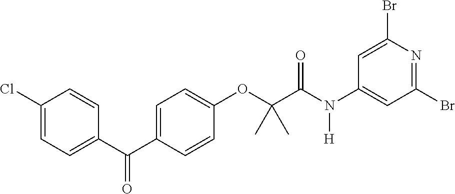 [SMILES: CC(C)(Oc1ccc(C(=O)c2ccc(Cl)cc2)cc1)C(=O)Nc1cc(Br)nc(Br)c1] | C₂₂H₁₇Br₂ClN₂O₃ | 552.64 |
| PP235 | 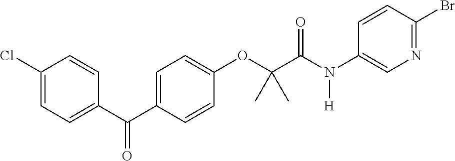 [SMILES: CC(C)(Oc1ccc(C(=O)c2ccc(Cl)cc2)cc1)C(=O)Nc1ccc(Br)nc1] | C₂₂H₁₈BrClN₂O₃ | 473.75 |
| PP237 | 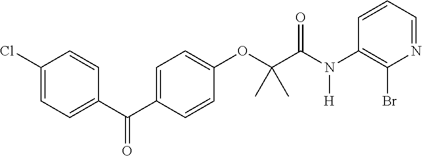 [SMILES: CC(C)(Oc1ccc(C(=O)c2ccc(Cl)cc2)cc1)C(=O)Nc1cccnc1Br] | C₂₂H₁₈BrClN₂O₃ | 473.75 |
| PP239 | 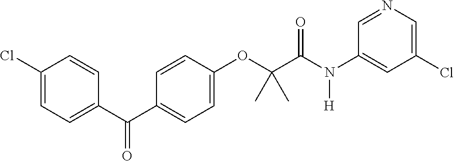 [SMILES: CC(C)(Oc1ccc(C(=O)c2ccc(Cl)cc2)cc1)C(=O)Nc1cncc(Cl)c1] | C₂₂H₁₈Cl₂N₂O₃ | 429.30 |
| PP240 | 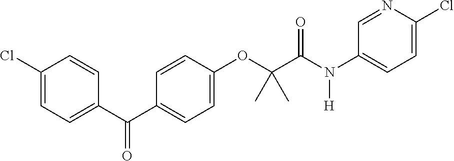 [SMILES: CC(C)(Oc1ccc(C(=O)c2ccc(Cl)cc2)cc1)C(=O)Nc1ccc(Cl)nc1] | C₂₂H₁₈Cl₂N₂O₃ | 429.30 |
| PP241 | 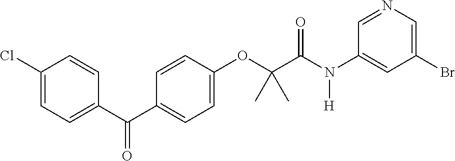 [SMILES: CC(C)(Oc1ccc(C(=O)c2ccc(Cl)cc2)cc1)C(=O)Nc1cncc(Br)c1] | C₂₂H₁₈BrClN₂O₃ | 473.75 |
| PP244 | 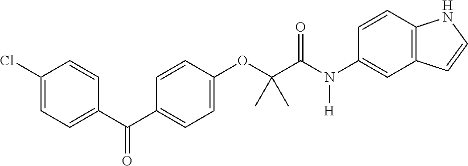 [SMILES: CC(C)(Oc1ccc(C(=O)c2ccc(Cl)cc2)cc1)C(=O)Nc1ccc2[nH]ccc2c1] | C₂₅H₂₁ClN₂O₃ | 432.90 |
| PP246 | 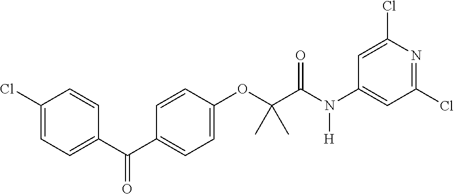 [SMILES: CC(C)(Oc1ccc(C(=O)c2ccc(Cl)cc2)cc1)C(=O)Nc1cc(Cl)nc(Cl)c1] | C₂₂H₁₇Cl₃N₂O₃ | 463.74 |
| PP247 | 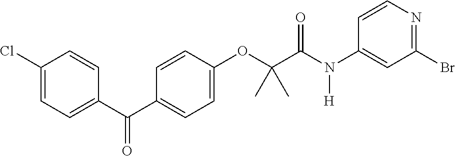 [SMILES: CC(C)(Oc1ccc(C(=O)c2ccc(Cl)cc2)cc1)C(=O)Nc1ccnc(Br)c1] | C₂₂H₁₈BrClN₂O₃ | 473.75 |
| PP248 | 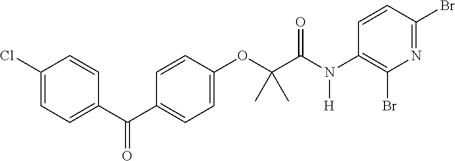 [SMILES: CC(C)(Oc1ccc(C(=O)c2ccc(Cl)cc2)cc1)C(=O)Nc1ccc(Br)nc1Br] | C₂₂H₁₇Br₂ClN₂O₃ | 552.64 |
| PP249 | 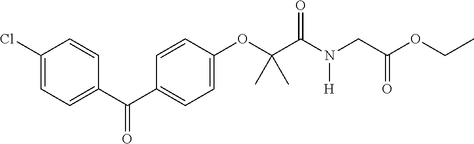 [SMILES: CCOC(=O)CNC(=O)C(C)(C)Oc1ccc(C(=O)c2ccc(Cl)cc2)cc1] | C₂₁H₂₂ClNO₅ | 403.86 |
| PP250 | 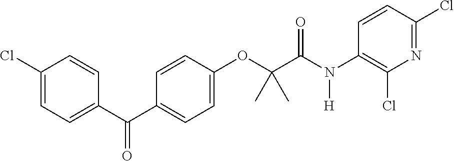 [SMILES: CC(C)(Oc1ccc(C(=O)c2ccc(Cl)cc2)cc1)C(=O)Nc1ccc(Cl)nc1Cl] | C₂₂H₁₇Cl₃N₂O₃ | 463.74 |
| PP251 | 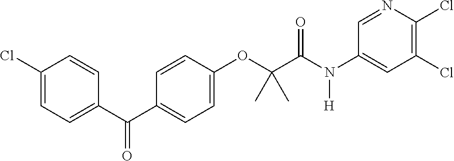 [SMILES: CC(C)(Oc1ccc(C(=O)c2ccc(Cl)cc2)cc1)C(=O)Nc1cnc(Cl)c(Cl)c1] | C₂₂H₁₇Cl₃N₂O₃ | 463.74 |
| PP252 | 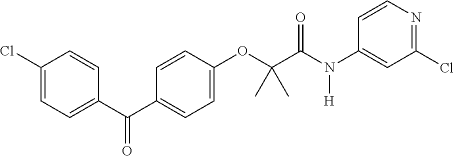 [SMILES: CC(C)(Oc1ccc(C(=O)c2ccc(Cl)cc2)cc1)C(=O)Nc1ccnc(Cl)c1] | C₂₂H₁₈Cl₂N₂O₃ | 429.30 |
| PP253 | 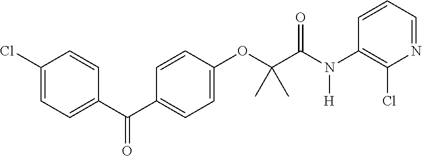 [SMILES: CC(C)(Oc1ccc(C(=O)c2ccc(Cl)cc2)cc1)C(=O)Nc1cccnc1Cl] | C₂₂H₁₈Cl₂N₂O₃ | 429.30 |
| PP256 | 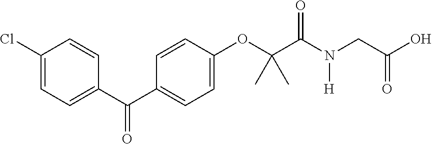 [SMILES: CC(C)(Oc1ccc(C(=O)c2ccc(Cl)cc2)cc1)C(=O)NCC(=O)O] | C₁₉H₁₈ClNO₅ | 375.80 |
| PP257 | 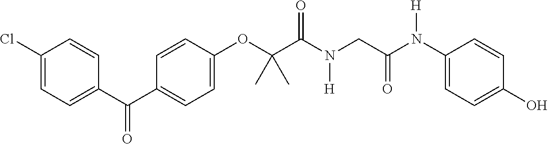 [SMILES: CC(C)(Oc1ccc(C(=O)c2ccc(Cl)cc2)cc1)C(=O)NCC(=O)Nc1ccc(O)cc1] | C₂₅H₂₃ClN₂O₅ | 466.91 |
| PP259 | 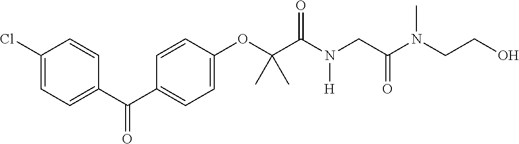 [SMILES: CN(CCO)C(=O)CNC(=O)C(C)(C)Oc1ccc(C(=O)c2ccc(Cl)cc2)cc1] | C₂₂H₂₅ClN₂O₅ | 432.90 |
| PP297 | 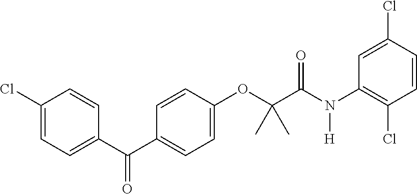 [SMILES: CC(C)(Oc1ccc(C(=O)c2ccc(Cl)cc2)cc1)C(=O)Nc1cc(Cl)ccc1Cl] | C₂₃H₁₈Cl₃NO₃ | 462.75 |
| PP298 | 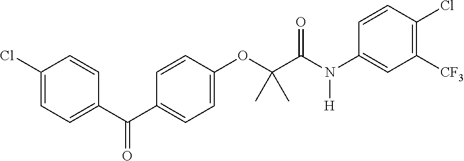 [SMILES: CC(C)(Oc1ccc(C(=O)c2ccc(Cl)cc2)cc1)C(=O)Nc1ccc(Cl)c(C(F)(F)F)c1] | C₂₄H₁₈Cl₂F₃NO₃ | 496.31 |
| PP299 | 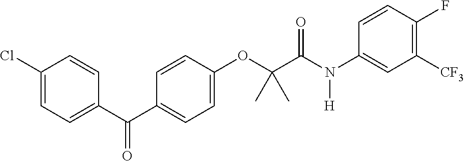 [SMILES: CC(C)(Oc1ccc(C(=O)c2ccc(Cl)cc2)cc1)C(=O)Nc1ccc(F)c(C(F)(F)F)c1] | C₂₄H₁₈ClF₄NO₃ | 479.85 |
| PP300 | 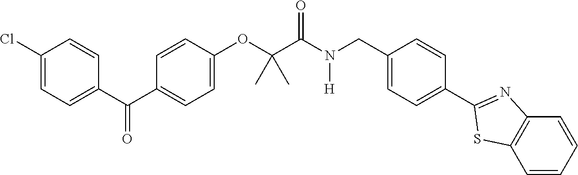 [SMILES: CC(C)(Oc1ccc(C(=O)c2ccc(Cl)cc2)cc1)C(=O)NCc1ccc(-c2nc3ccccc3s2)cc1] | C₃₁H₂₅ClN₂O₃S | 541.06 |
| PP304 | 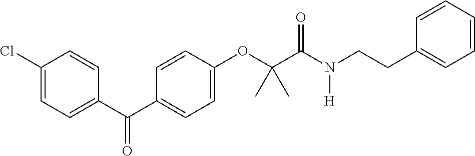 [SMILES: CC(C)(Oc1ccc(C(=O)c2ccc(Cl)cc2)cc1)C(=O)NCCc1ccccc1] | C₂₅H₂₄ClNO₃ | 421.92 |
| PP305 | 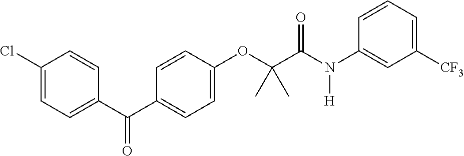 [SMILES: CC(C)(Oc1ccc(C(=O)c2ccc(Cl)cc2)cc1)C(=O)Nc1cccc(C(F)(F)F)c1] | C₂₄H₁₉ClF₃NO₃ | 461.86 |
| PP306 | 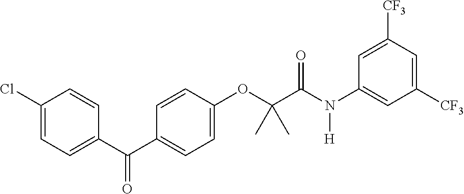 [SMILES: CC(C)(Oc1ccc(C(=O)c2ccc(Cl)cc2)cc1)C(=O)Nc1cc(C(F)(F)F)cc(C(F)(F)F)c1] | C₂₅H₁₈ClF₆NO₃ | 529.86 |
| PP307 | 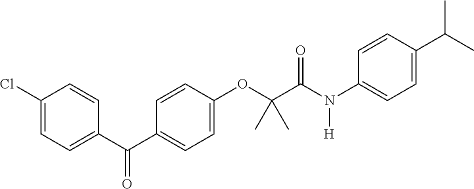 [SMILES: CC(C)c1ccc(NC(=O)C(C)(C)Oc2ccc(C(=O)c3ccc(Cl)cc3)cc2)cc1] | C₂₆H₂₆ClNO₃ | 435.94 |

### Example 5

Exploring Anticancer Activity of structurally Modified Benzoylphenoxyacetamide (BPA) II: Synthesis Strategies and Computational Analyses of Phenolic BPAs with High Anti-Glioblastoma Potential.

Extensive computational studies of phenol and naphthol modifying benzoylphenoxyacetamide (BPA) were performed with target to generated physicochemical properties that can be used as to select several molecular skeletons for preparation and study with target to develop amt-glioblastoma drug candidate. Overall extensive computational studies of 71 structural variants of BPA were performed and their physicochemical properties such as solubility (log S), brain-blood partitioning (log BB) and probability to penetrate central nervous system (MPO-CNS), polar surface area, molecular polarizability, electrostatic surface maps, and frontier orbitals were evaluated. From these set of generated data statistical cut off values were used to select eighteen BPA based candidates with phenol and naphthol moieties be prepared and experimentally evaluated their anti-glioblastoma activity. Nine of these compounds show acceptable anti-glioblastoma activity at 25 μM concentration on LN229 cell line. However only four (HR49, HR50, HR51, HR59) are selected as anti-glioblastoma drug candidates considering that for these compounds acceptable water solubility and brain penetration were calculated. In addition, their IC50 values are below 10 μM. These compounds will be further evaluated as anti-glioblastoma drug candidates.

Introduction

Glioblastoma (GBM) is aggressive malignant tumor in adult brain and one of the most challenging malignancies for treatment in the oncology [Holland, E, C. “Glioblastoma multiforme: The terminator” PNAS 2000, 97, 6242-6244]. Standard therapeutic approach to treat GMB for many years has been surgical resection and postoperative radiotherapy. This standard treatment has resulted in a poor median survival of about 12 months [Weller M, van den Bent M, Tonn J C, Stupp R, Preusser M, Cohen-Jonathan-Moyal E, Henriksson R, Le Rhun E, Balana C, Chinot O, Bendszus M, Reijneveld J C, Dhermain F, French P, Marosi C, Watts C, Oberg I, Pilkington G, Baumert B G, Taphoorn M J B, Hegi M, Westphal M, Reifenberger G, Soffietti R, Wick W; “European Association for Neuro-Oncology (EANO) Task Force on Gliomas” Lancet Oncol. 2017, 18, e315-e329]. Addition temozolomide (TMZ) to surgery and radiotherapy has become the standard first-line treatment for GBM, but with an increase of the median survival for only about 2.5 months [Jaoude, D. A.; Moore, J. A.; Moore, M. B.; Twumasi-Ankrah, P.; Ablah, E.; Moore, D. F. “Glioblastoma and Increased Survival with Longer Chemotherapy Duration” Kans. J. Med. 2019, 12, 65-69.]. Considering that there are many FDA-approved drugs [As of May 2019 the FDA approved drugs for brain cancer are Afinitor (Everolimus), Afinitor Disperz (Everolimus), Avastin (Bevacizumab), Bevacizumab), NICNU (Carmustine), Carmustine Implant, Everolimus, Gliadel Wafer (Carmustine Implant), Lamustine, Mvasi (Bebvacizumab), Temodar (Temozolomide), Temozolomide] with one drug combination (PCV combination include Procarbazzine Hydrochloride, Lomustine, Vincristine Sulfate) for cancer treatment and that there is also noticeable progress made in the molecular and cellular profiling of GBM the increase of the survival moderate [Paolollo, M.; Boselli, C.; Schinelli, S. “Glioblastoma under Siege: An Overview of Current Therapeutic Strategies” Brain Sci. 2018, 8(1): 15.]

An efficient treatment of GBM is difficult to develop for a series of reasons. First, GBM is characterized by many dysregulated pathways that can hardly be all blocked and repaired at the same time with a single therapy [Alifieris, C.; Trafalis, D. T. “Glioblastoma multiforme: pathogenesis and treatment” Pharmacol. Ther. 2015, 152, 63-82.]. Second, GBM partly consists of infiltrating cells that cannot easily be all removed by surgery. Third, GBM early diagnosis is not carried out routinely. Sensitive imaging techniques, such as MRI are still too expensive to be carried out on a regular basis over the whole population. Fourth, the optimization of a clinical protocol for GBM treatment requires the use of an accurate and representative preclinical GBM model. Currently used mouse and rat models are not appropriate because their tumors are typically ˜10³-10⁴ smaller than human GBM [Biasibetti, E.; Valazza, A.; Capucchio, M. T.; Annovazzi, L.; Battaglia. L.; Chirio, D.; Gallarate, M.; Mellai, M.; Muntoni, E.; Peira, E.; Riganti, C.; Schiffer, D.; Panciani, P.; Lanotte, M. “Comparison of Allogeneic and Syngeneic Rat Glioma Models by Using MRI and Histopathologic Evaluation” Comp. Med. 2017, 67, 147-156.]. Fifth, the blood brain barrier (BBB) often prevents drugs from efficiently reaching glioblastoma cells, and methods to enable drugs to efficiently cross the BBB should therefore be developed [Harder, B. G.; Blomquest, M. R.; Wang, J.; Kim, A. J.; Woodworth, G. F.; Winkles, J. A.; Loftus, J. C.; Tran, N. L. “Developments in Blood-Brain Barrier Penetrance and Drug Repurposing for Improved Treatment of Glioblastoma” Front. Oncol. 2018, 8, 462]. In fact, there are actually 3 structural variation that are commonly used as anti-glioblastoma drugs: Temozolomine (Temodar, TMZ), Lomustine and Carmustine as chloroethylnitrosoureas, Avastin (Bevacizumab, Mvasi) as monoclonal antibody. TMZ is rapidly bsorbed and eliminated [Agarwala, S. S.; Kirkwood, J. M. “Temozolomide, a Novel Alkylating Agent with Activity in the Central Nervous System, May Improve the Treatment of Advanced Metastatic Melanoma” The Oncologist 2000, 5, 144-151.]. Upon oral administration maximum plasma concentration was reach less the one our and the elimination half-life is approximately 1.8 hours. Penetration of TMZ into CNS has been studied in 35 patients with newly diagnosed or recurrent malignant gliomas showing that the drug concentration of the drug in brain and cerebrospinal fluid is approximately 20% of the plasma concentration [Ostermann, S.; Csajka, C.; Buclin, T.; Leyvraz, S.; Lejeune, F.; Descosterd, L. A.; Stupp, R. “Plasma and Cerebrospinal Fluid Population Pharmacokinetics of Temozolomidine in Malignant Glioma Patients” Clin. Cancer Res. 2004, 100, 3728-36]. This makes experimentally estimated log BB (Brain-Blood Distribution) to be around −0.7.

It is quite clear that none of these therapeutic strategies are producing actable response. There are more and more studies that include most widely used anti-glioblastoma drug, TMZ in combination with other drugs [Gao, L.; Huang, S.; Zhang, H.; Hua, W.; Xin, S.; Cheng, L.; Guan, W.; Yu, Y.; Mao, Y.; Pei, G. “Suppression of glioblastoma by a drug cocktail reprogramming tumor cells into neuron like cells” Nature Scientific Reports 2019, 9, 3462]. One of the approaches is combination of TMZ with lipid lowering drugs [Vasilev, A.; Sofi, R.; Tong, L.; Teschemascher, A. G.; Kasparov, S. “In search of a Breakthrough Therapy for Glioblastoma Multiforme” Neuroglioma 2018, 1, 292-310.] One lipid-lowering drug that has attracted attention as a candidate for an anticancer regimen is fenofibrate (FF) [Majeed, Y.; Upadhayay, R.; Alhousseiny, S.; Taha, T.; Musthak, A. Shaheen, Y.; Jameel, M.; Triggle, C. R.; Ding, H. “Potent and PPARalfa-independent anti-proliferative action of the hypolipidemic drug fenofibrate in VEGF-dependent angiosarcomas in vitro” Nature Scientific Reports 2019, 9, 6316.] Recently we have demonstrated that 50 μM FF has a strong anticancer activity with low systemic toxicity [Grabacka, M; Waligorski, P.; Zapata, A.; Blake, D. A.; Wyczechowska, D.; Wilk, A.; Rutkowska, M.; Vishistha, H.; Ayyala, R.; Ponnusamy, T.; John, V. T.; Culicchia, F.; Wisniewska-Becker, A.; Reiss, K. “Fenofibrate Subcellular Distribution as a Rational for the Intercranial Delivery Through Biodegradable Carrier” J. Physiol. Pharmacol. 2015, 66, 233-247.]. However, FF does not cross the BBB, and is quickly processed by the blood and tissue esterases to form fenofibric acid (FFA) Wilk, A.; Wyczechowska, D.; Zapata, A.; Dean, M.; Mullinax, J.; Marrero, L.; Parsons, C.; Peruzzi, F.; Culicchia, F.; Ochoa, A.; Grabacka, M.; Reiss, K. “Molecular Mechanism of Fenofibrate-Induced Metabolic Catastrophe and Glioblastoma Cell Death” Mol. Cell. Biol. 2015, 35, 182-198.]. This acid is no longer effective in triggering tumor cell death.

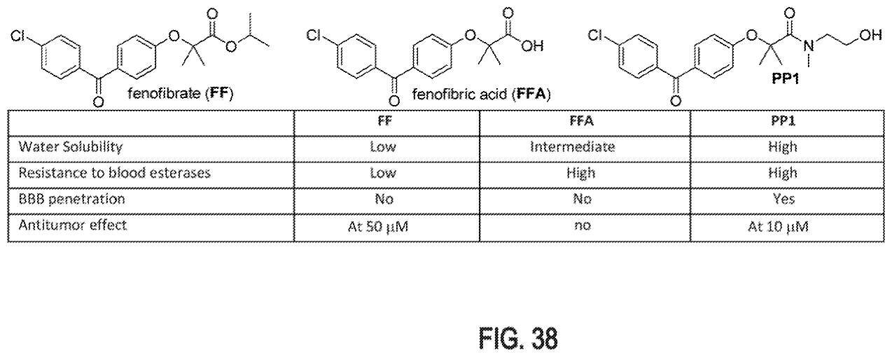

To eliminate both problem with hydrolysis as well as to increase solubility, tissue penetration and ultimately anti-glioblastoma activity we have made several chemical modifications to the FF molecular skeleton. One of these modifications has resulted in a new compounds, PP1 (FIG. 38), that triggers extensive glioblastoma cell death in vitro at concentrations almost 5-fold lower than FF. However both of these compounds (FF and PP1) inhibited mitochondrial respiration but PP1 has higher water solubility, better BBB penetration, and better resistance to blood esterases [Stalinska, J.; Zimolag, E.; Pianovich, N. A.; Zapata, A.; Lassak, A.; Rak, M.; Dean, M.; Ucar-Bilyeu, D.; Culicchia, F.; Marrero, L.; Del Valle, L.; Sarkaria, J.; Peruzzi, F.; Jursic, B. S.; Reiss, K. “Chemically Modified Variants of Fenofibrate with Antiglioblastoma Potential” Trans. Oncol. 2019, 12, 895-907.]. According to our HPLC measurement the accumulation of PP1 in the brain tumor tissues varied between 5 and 6 μM, that might not be sufficient since it is highly effective at 10. In our following studies we have developed derivatives based on benzoylphenoxyacetamide (BPA) molecular skeleton and we have developed a new structural model for design and exploring anti-glioblastoma activity. This was resulted in several potent anti-glioblastoma drug candidates with hydroxyalkyl/ether BPA moieties. To improve anti-glioblastoma activity and obtain desirable blood-brain penetration and water solubility new phenol derivatives of BPA were explored [Abotaleb, M.; Sauel, S. M.; Varghese, E.; Varghese, S.; Kubatka, P.; Liskova, A.; Busselberger, D. “Flavonoids in Cancer and Apoptosis” Cancers 2019, 11, 28.; Bonta, R. K.; “Dietary Phenolic Acids and Flavonoides as Potential Anti-cancer Agents: Current State of Art and Future Perspectives” Anticancer Agents Med. Chem. 2019.] As a result, 18 additional compounds were generated and analyzed in this study.

Results and Discussion

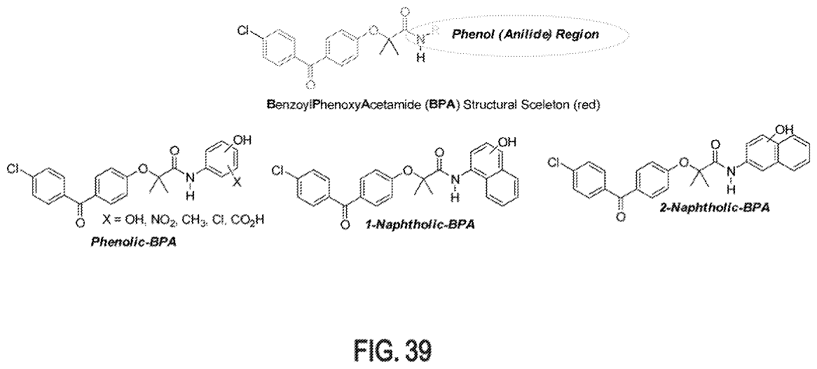

Overall Chemical Design of Therapeutic Compounds: In our previous studies we have explore importance of basic BPA skeleton and we have demonstrated that this is the pharmacophore [Gao, Q.; Yang, L.; Zhu, Y. “Pharmacophore based drug design approach as a practical process in drug discovery” Curr. Comput Aided Drug Des. 2010, 6, 37-49.] necessary to retain anti-glioblastoma activity. Amide part of the BPA skeleton can be modified in such a way to obtain desirable biological activity in combination with acceptable physicochemical molecular properties. We have selected phenol and naphthol BPA variants because phenol derivatives, besides many other health benefits, can have anti-cancer activity {Dzialo, M.; Mierziak, J.; Korzun, U.; Preisner, M.; Szopa, J.; Kulma, A. “The Potential of Plant Phenolics in Prevention and Therapy of Skin Disorders” Int. J. Mol. Sci. 2016, 17, 160; Bhuyan, J.; Basu, A. “Phenolic Compounds Potential Health Benefits and Toxicity” in Utilization of Bioactive Compounds from Agricultural and Food Waste. Chapter 2, Editor: Voug, Q. V. CRC Press, 2017]. The basic molecular skeleton for all prepared and tested compounds in this paper therefore contains phenolic BPA skeleton (FIG. 39). There are three seats of phenolic BPA: one single substituted phenols (Phenolic-BPA), and two naphtholic BPAs (1-Naphtholic-BPA and 2-Naphtholic-BPA) (FIG. 39).

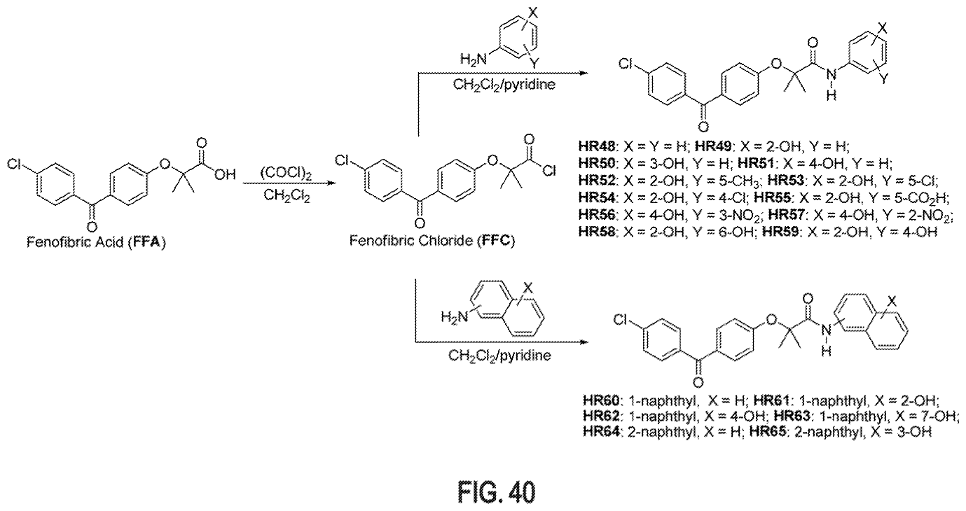

The starting point for the preparation of each and all phenolic BPA starts with readily available fenofibric acid (FFA) and corresponding aminophenol or aminonaphthol (FIG. 40) through an amide (peptide) coupling reactions [Brown, D. G.; Bostrom, J. “Analysis of Past and Present Synthetic Methodology on Medicinal Chemistry: Where Have All the New Reactions Gone?” J. Med. Chem. 2016, 59, 4443-4458.; El-Faham, A.; Albericio, F. “Peptide Coupling Reagents, More than a Letter Soup” Chem. Rev. 2011, 111, 6557-6602.; Pattabiraman, Vijaya R. Bode, Jeffrey W. “Rethinking Amide Bond Synthesis” Nature, 2011, 480, 471-479]. As we have previously reported [Stalinska, J H.; Houser, L.; Rak, M.; Colley, S. B.; Reiss, K.; Jursic, B. S. “Exploring anticancer activity of structurally modified benzylphenoxyacetamide (BPA); I Synthesis strategies and computational analyses of substituted BPA variants with high anti-glioblastoma potential” Scientific Reports 2019, 9, 17021.] due to steric hindrance of carboxylic group of FFA (two methyl groups in alpha position of carboxylic acid) in combination with poor amine nucleophilicity DCC or EDC coupling reagents are not producing acceptable isolated yields [Due-Hansen, M. E.; Pandey, S. K. Christiansen, R.; Hansen, S. V.; Ulven, T. “A protocol for amide bond formation with electron deficient amides and sterically hindered substrates” Org. Biomol. Chem. 2016, 14, 430-433.]. One would expect that aminophenol which are stronger nucleophile in comparison with nonactivated anilines [Acton, A. “Aniline Compounds—Advances in Research and Application” Scholarly Editions, Atlanta, Ga. 2012.] and EDC or DCC should produce corresponding BPA compounds in acceptable yield. Here again we were able to detect traces of the desirable products and this method of preparation of phenolic BPAs was not applicable. As in previous cases FFA was converted into more reactive fenofibrate chloride (FFC) followed by coupling with aminophenols or aminonaphthols (FIG. 40). The FFC was prepared fresh and immediately used without storing in next step reaction with aminophenols. Most common reagent for preparation of acid chloride is thionyl chloride that requires heating thionyl chloride solution of corresponding acid with appropriate traps for hydrochloric acid and sulfur dioxide as toxic and corrosive biproducts [Allen, C. F. H.; Byers, Jr. A. J. R.; Humphlett, W. J. “Oleoyl Chloride” Organic Syntheses, 1957, Coll. Vol. 4., 66.]. Second, mild (room temperature) but also producing corrosive and toxic gases [Montalbetti, C. A. G. N.; Falque, V. “Amide bond formation and peptide coupling” Tetrahedron, 2005, 10827-10852] is the Vilsmeier-Haack reaction using oxalyl chloride with catalytic amount of N,N-dimethylformamide (DMF) [Kimura, Y.; Matsuura, D. “Novel Synthetic Method for the Vilsmeier-Haack Reagent and Green Routes to Acid Chlorides, Alkyl Formates, and Alkyl Chlorides” International Journal of Organic Chemistry, 2013, 3, 1-7.]. Corrosive (HCl) and toxic (CO) by products of the reaction must also be trapped. This method was used for preparation of FFC. Upon preparation and removal of excess of oxalyl chloride under flow of dry argon the chloride was dissolved in dichloromethane (2 ml of dichloromethane for 1 mmol of FFC) and injected in the pyridine solution of corresponding aminophenol or aminonaphthol. Having in mind that at some point in our process to bring some of these compounds closer to the market as anti-glioblastoma agent simplified and large-scale preparation procedure must be developed. Considering this target, we have developed synthetic methodology that is capable to produce large quantity of pure product avoiding expensive and time-consuming extractions and chromatography (FIG. 40).

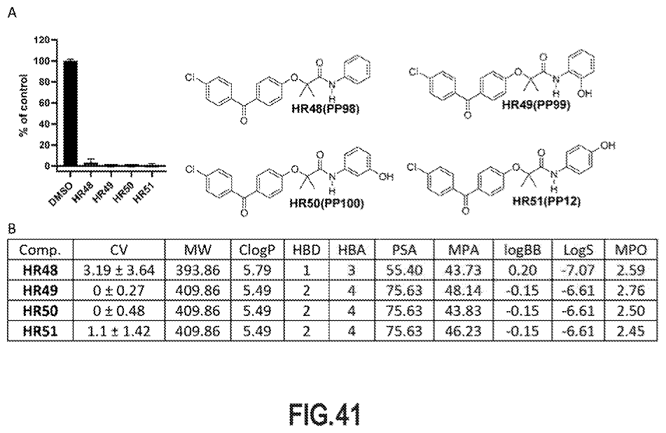

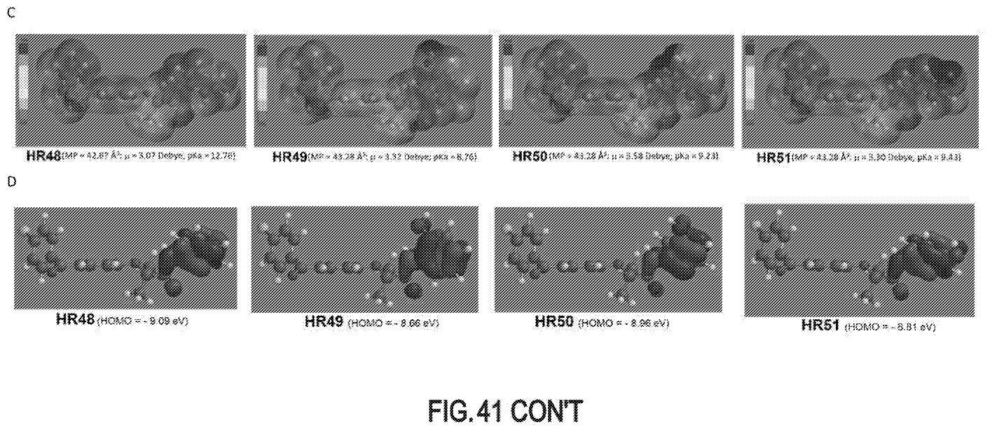

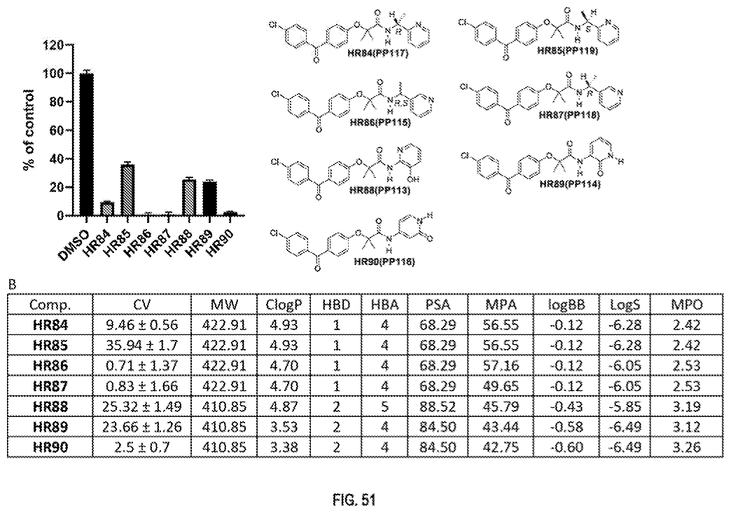

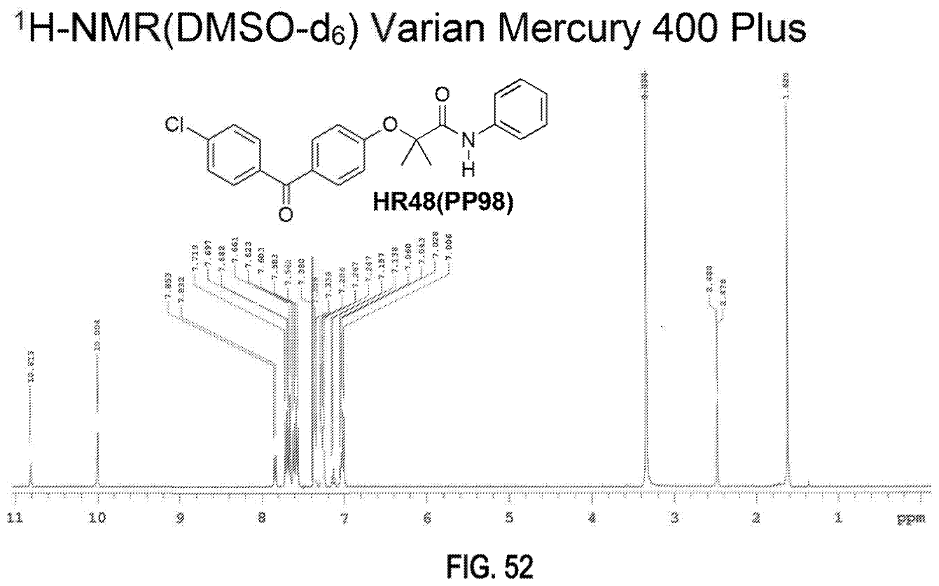

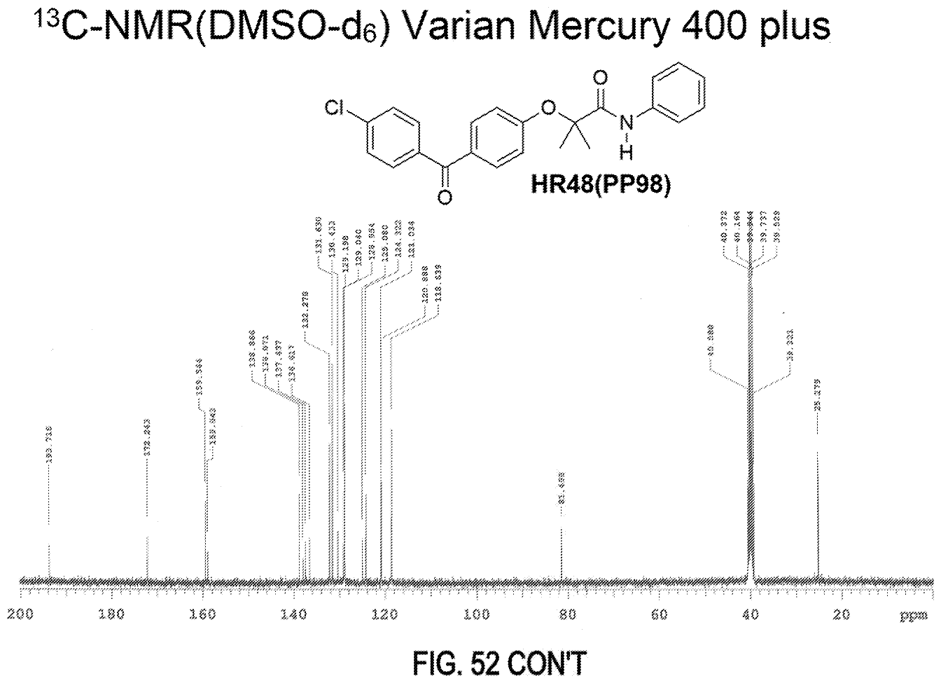

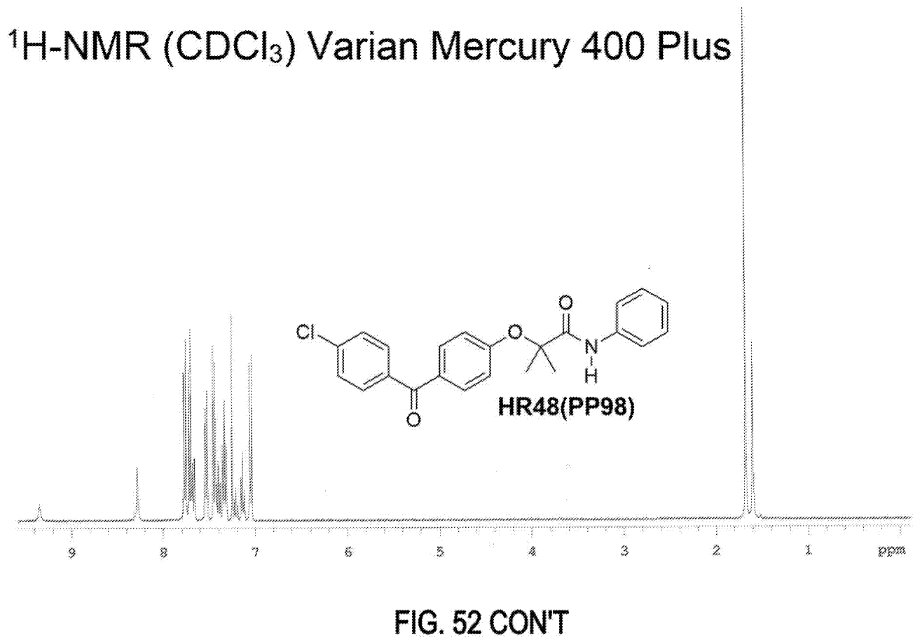

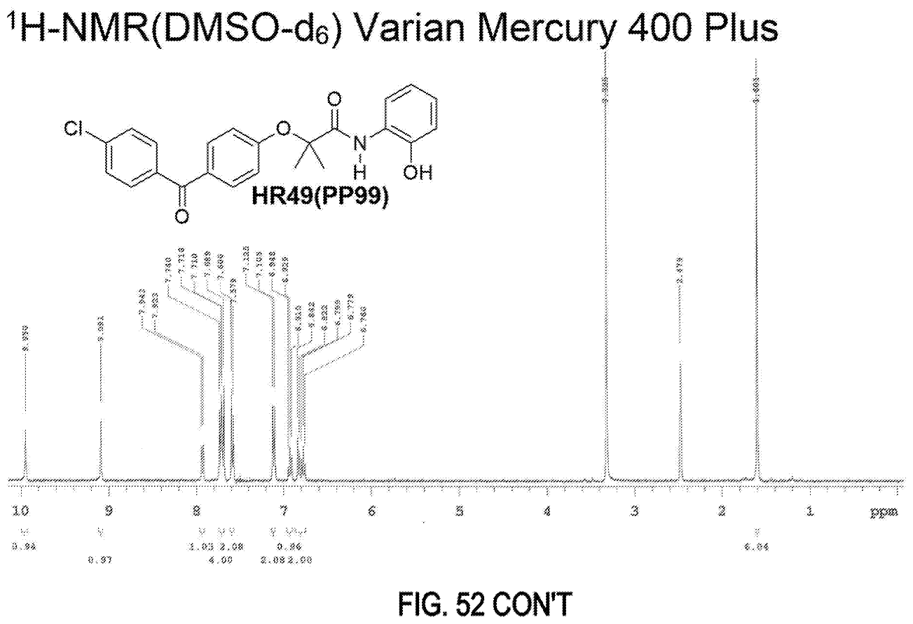

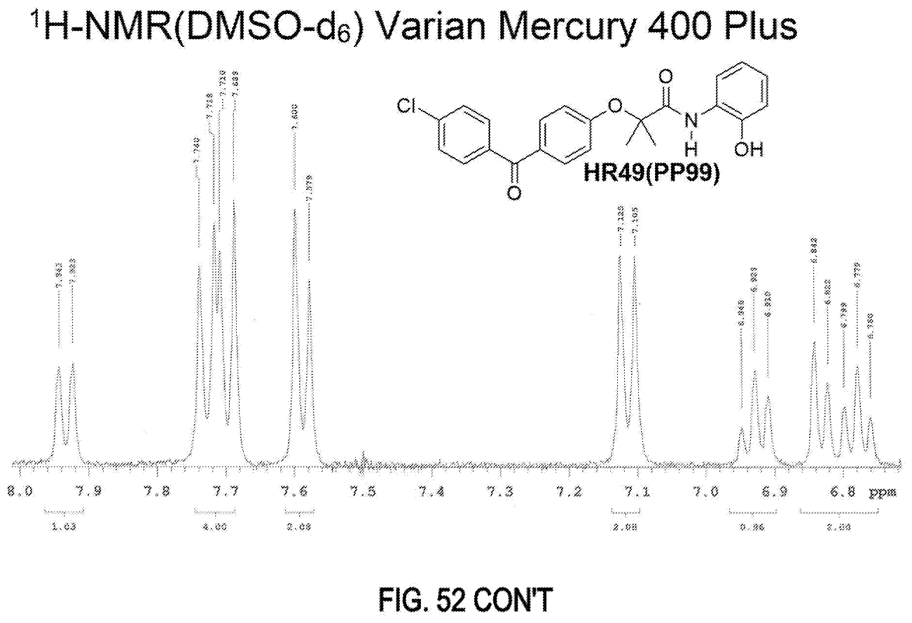

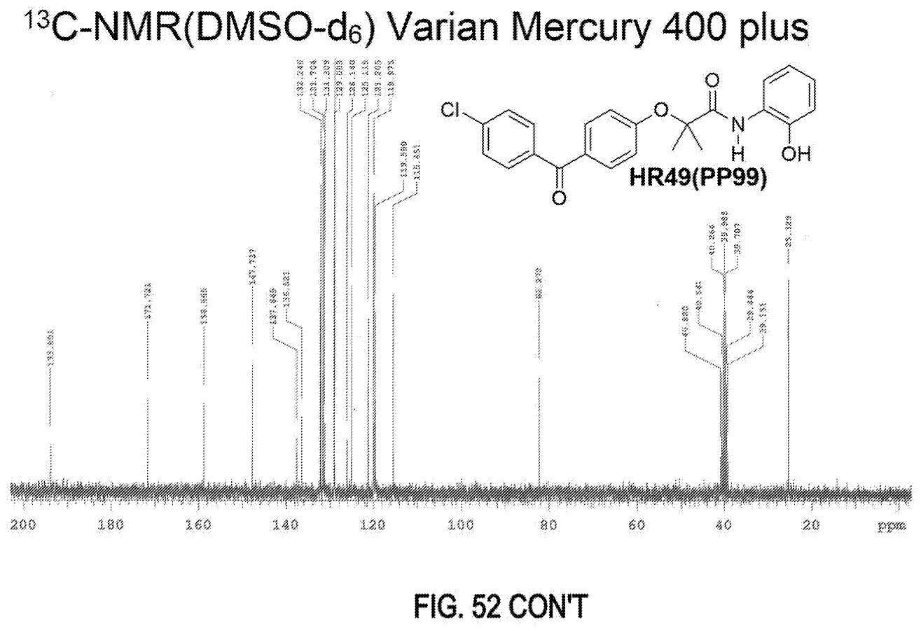

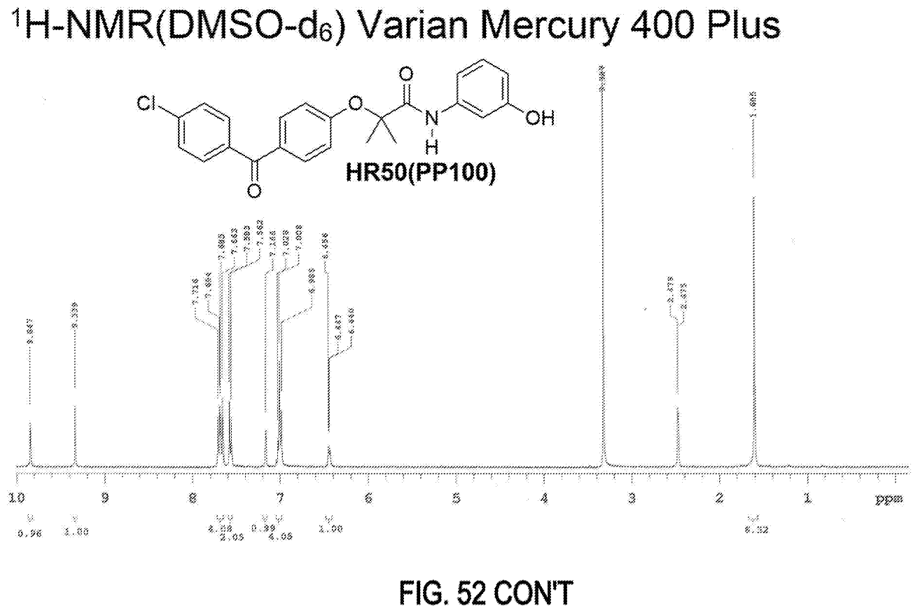

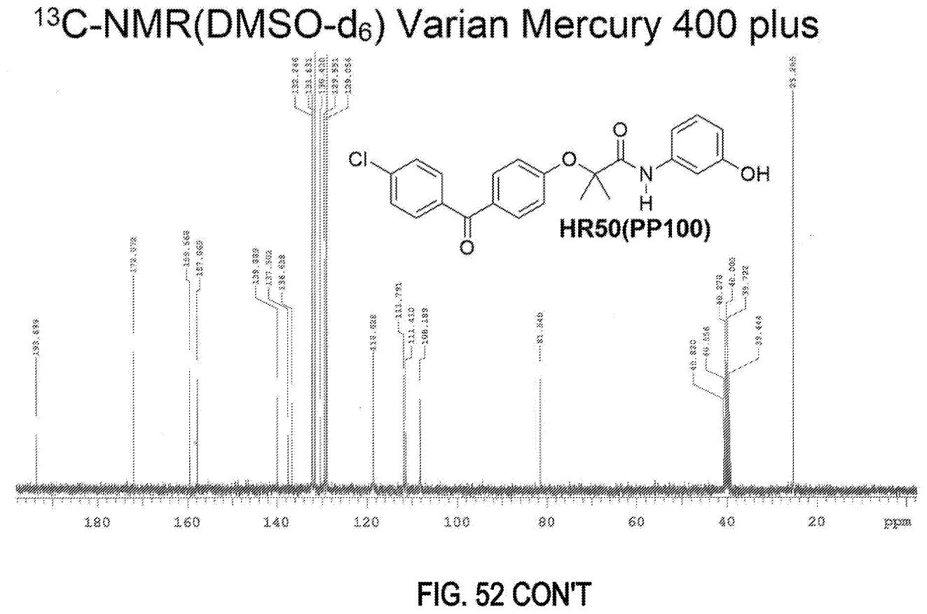

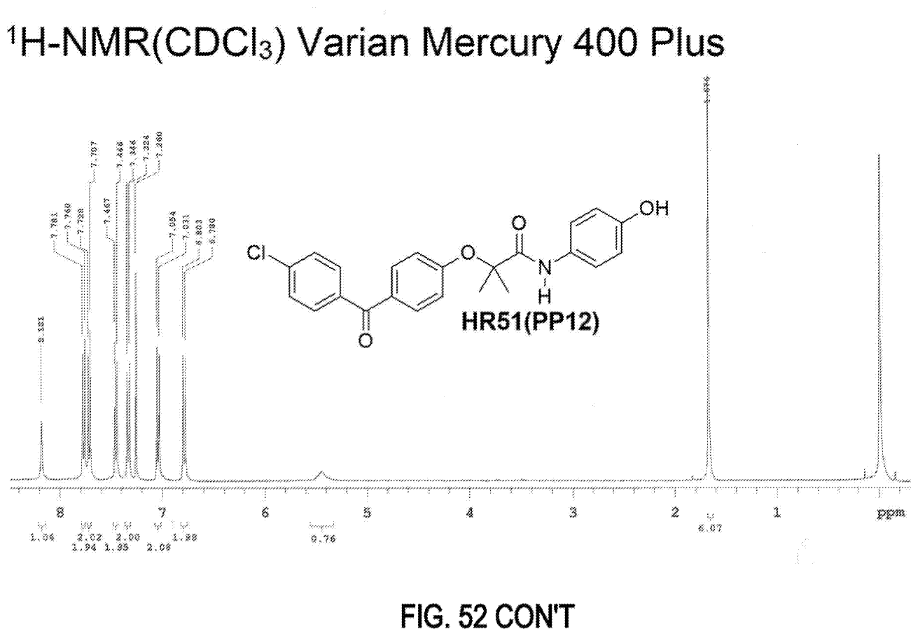

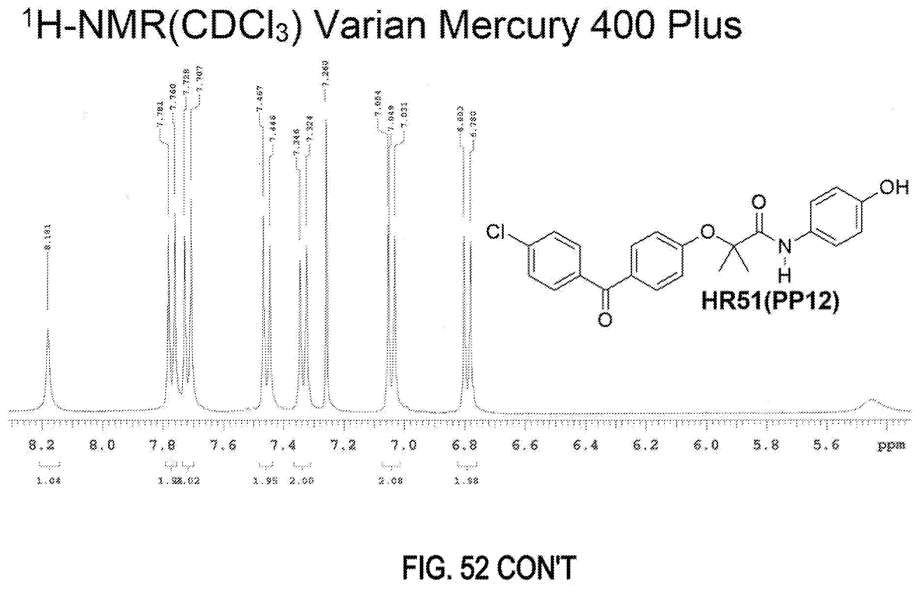

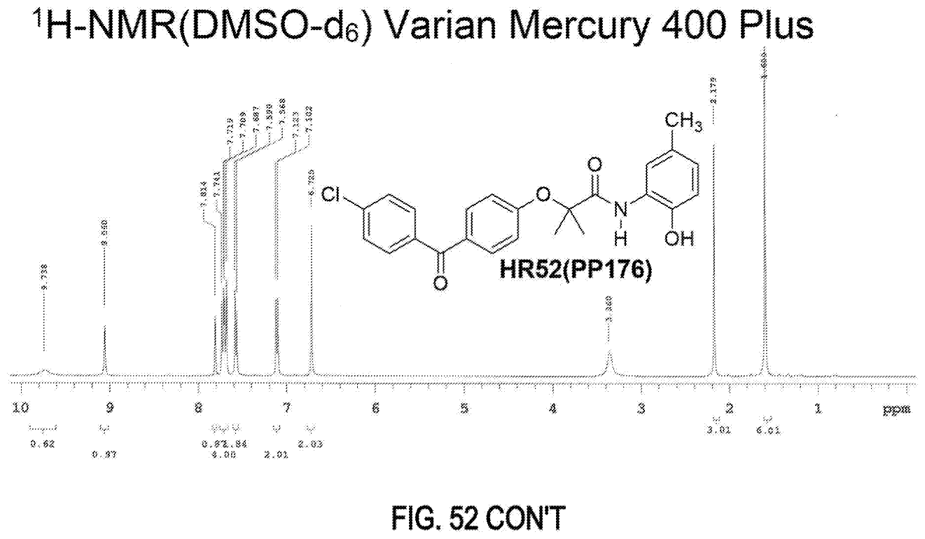

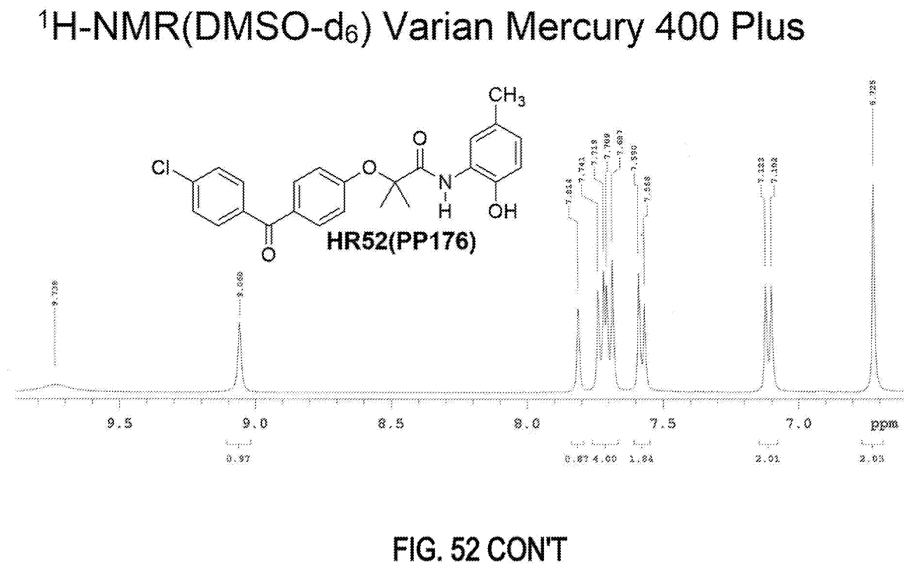

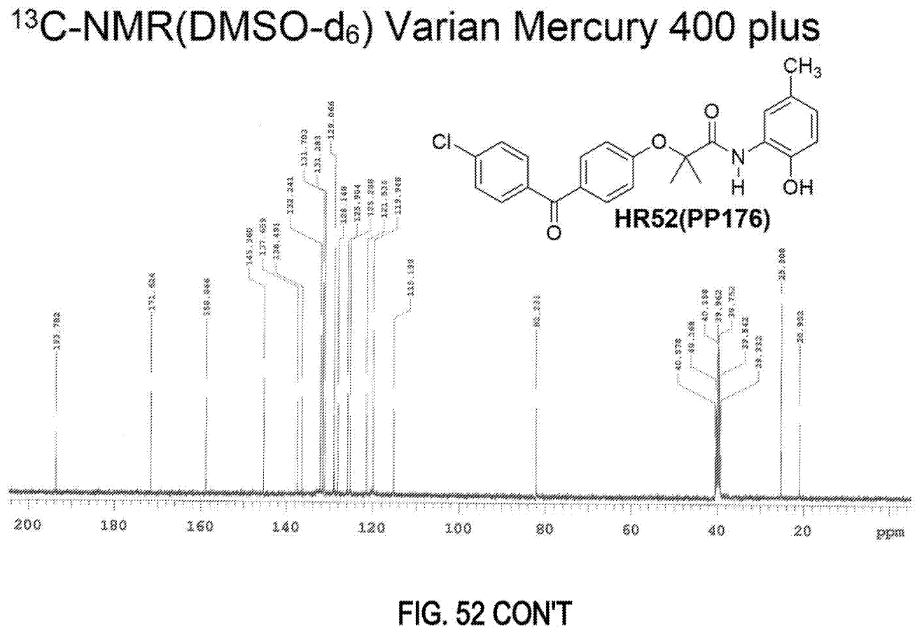

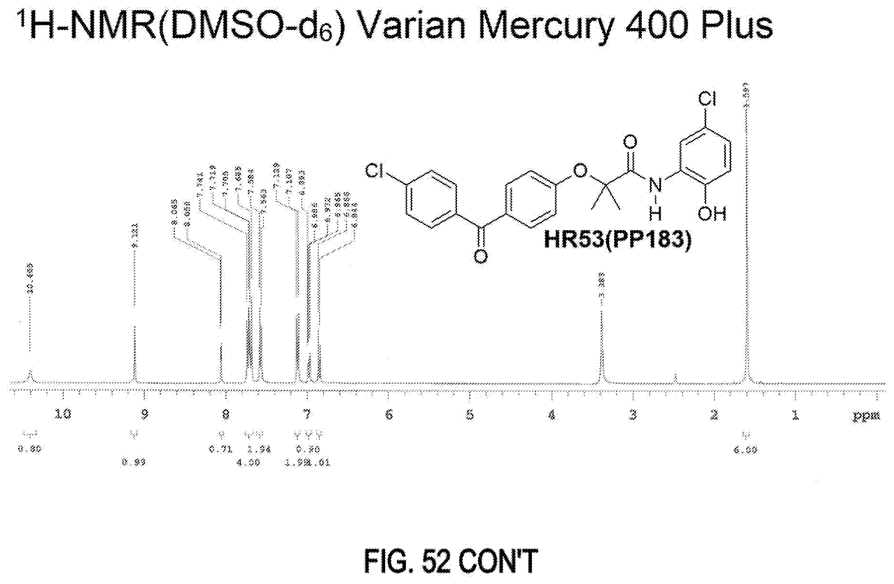

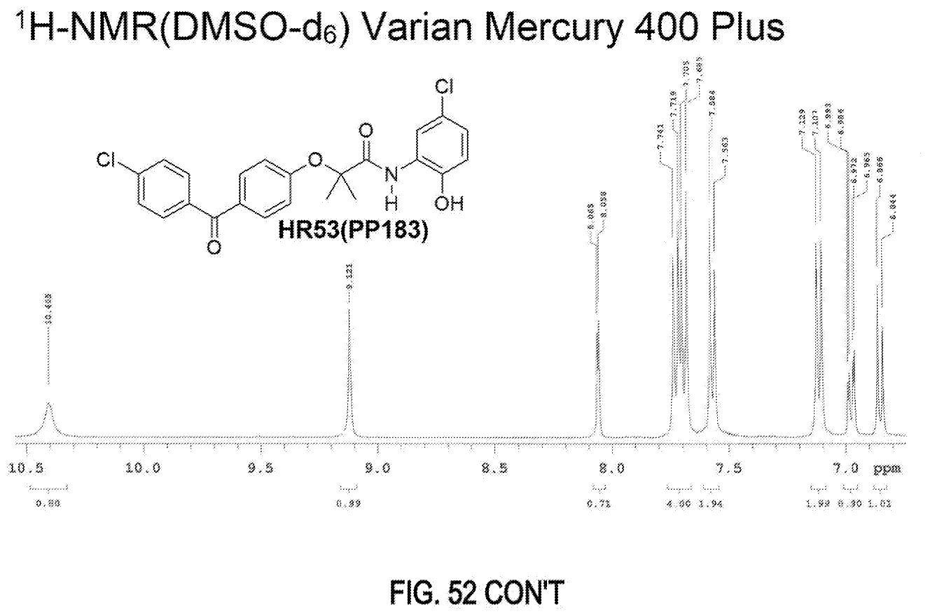

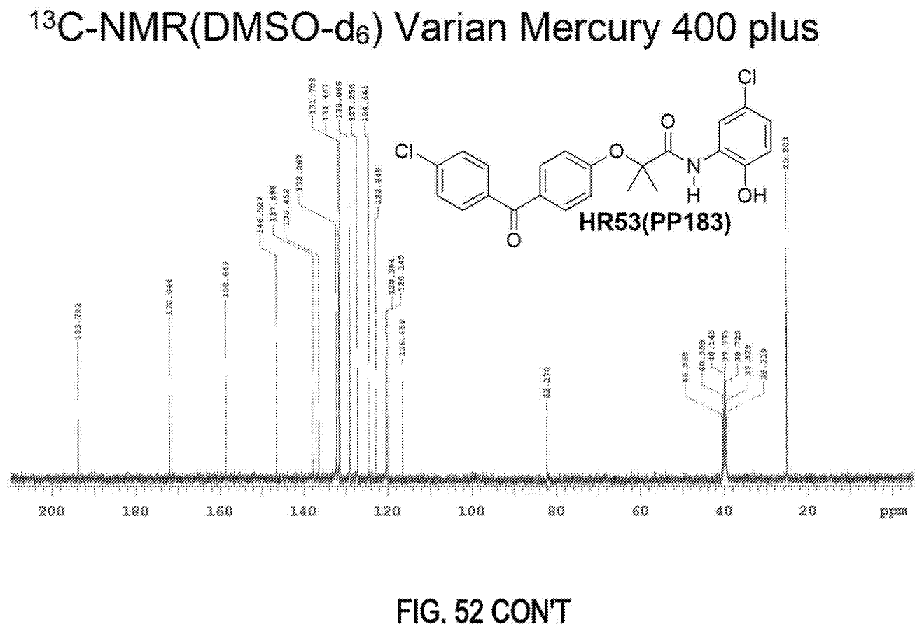

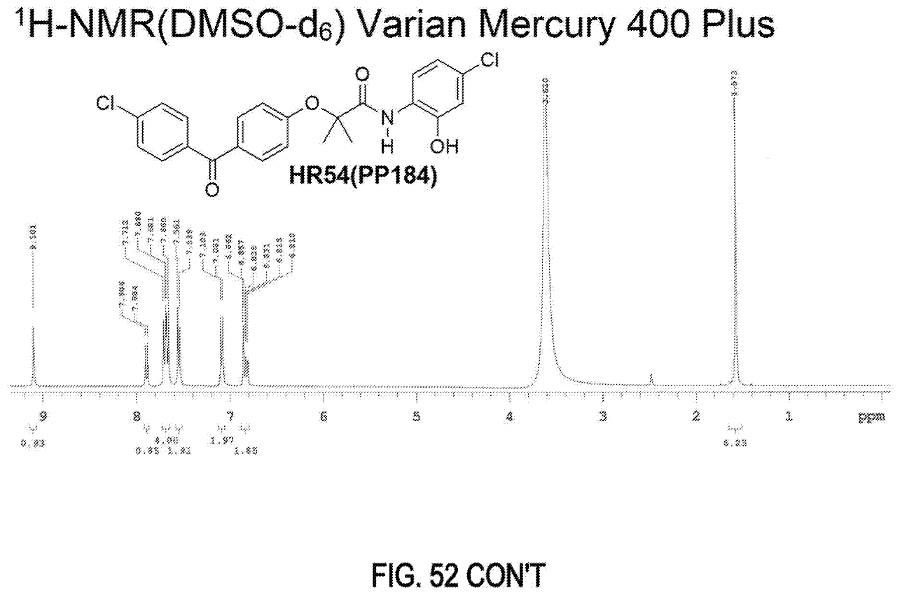

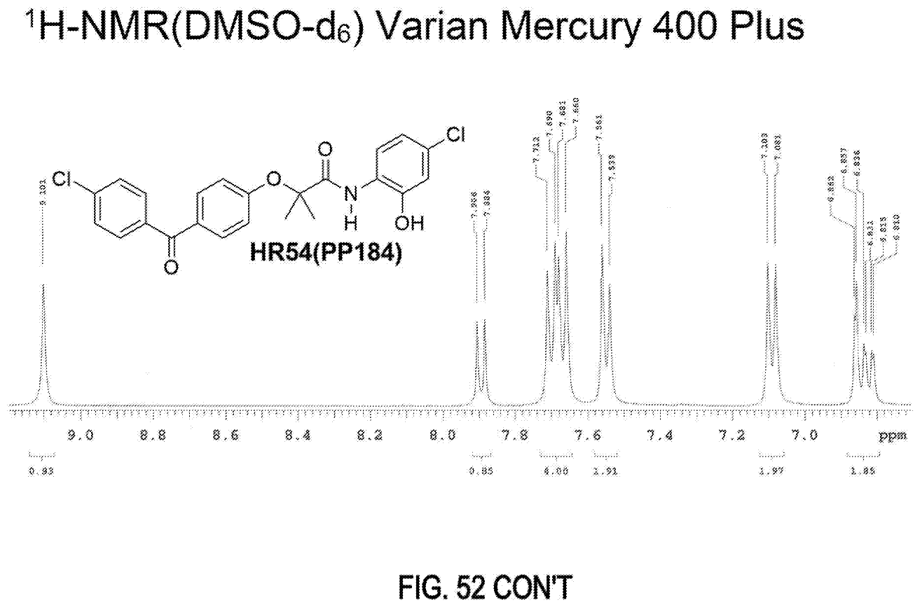

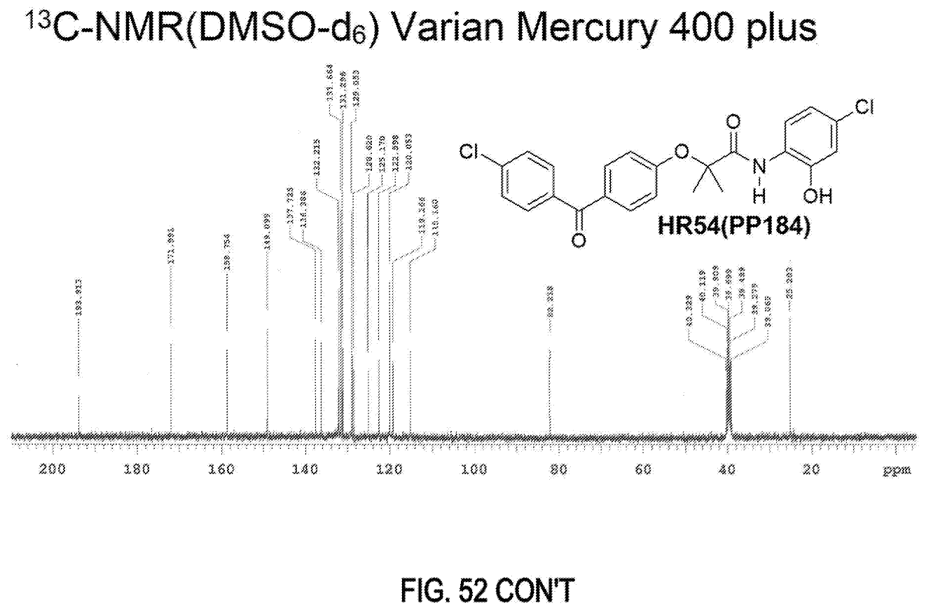

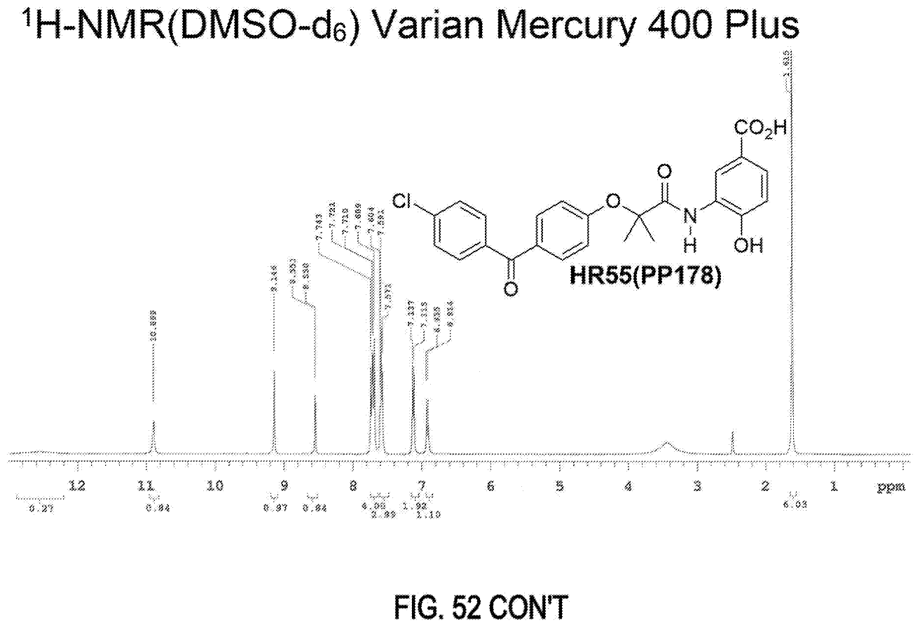

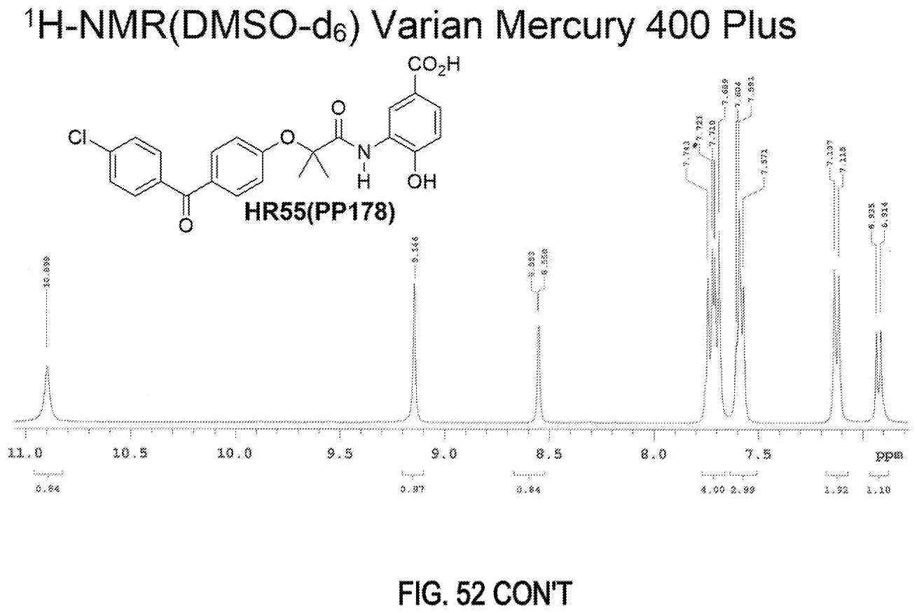

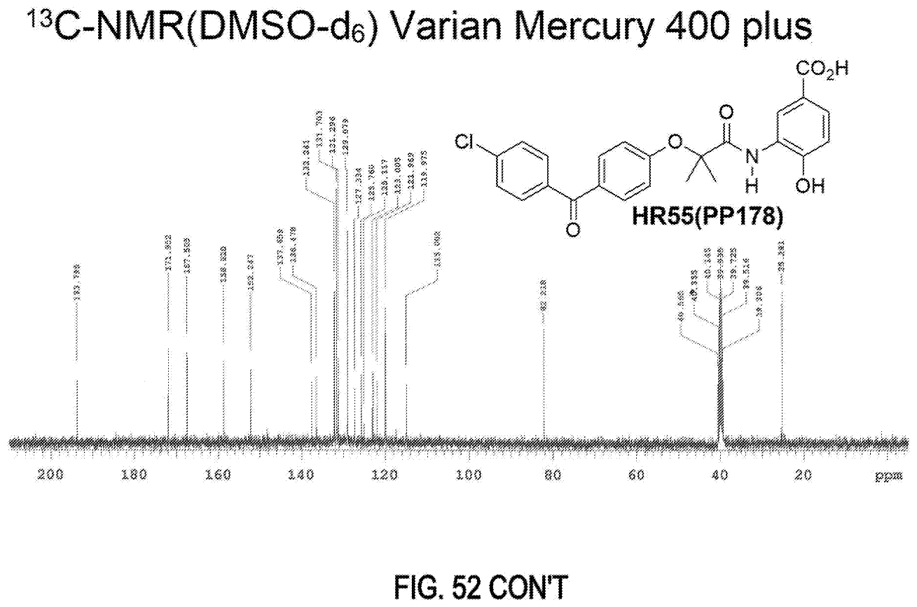

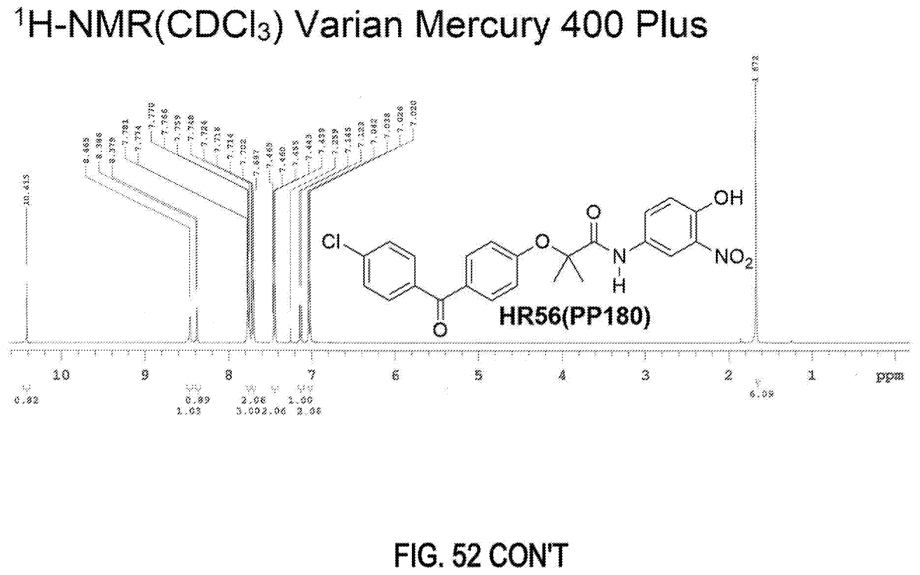

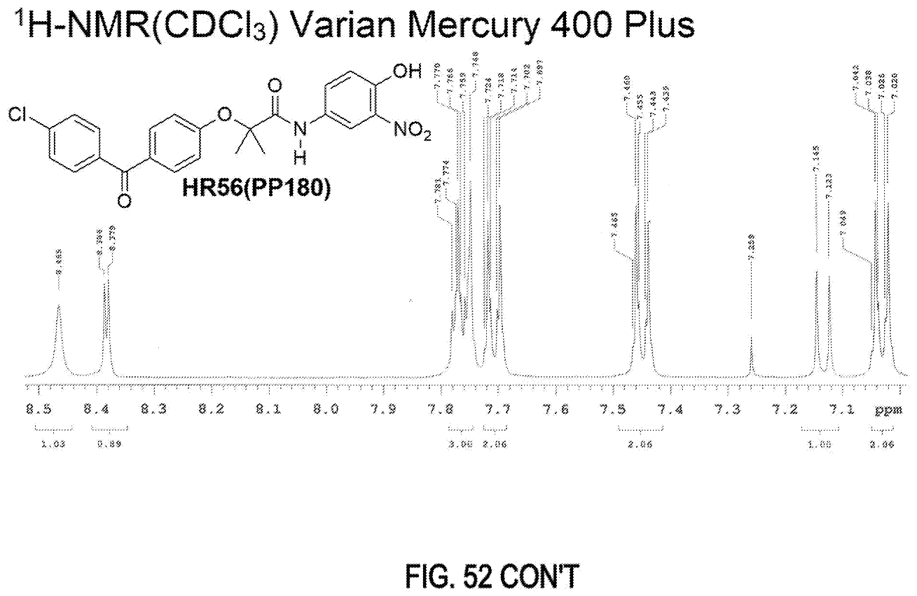

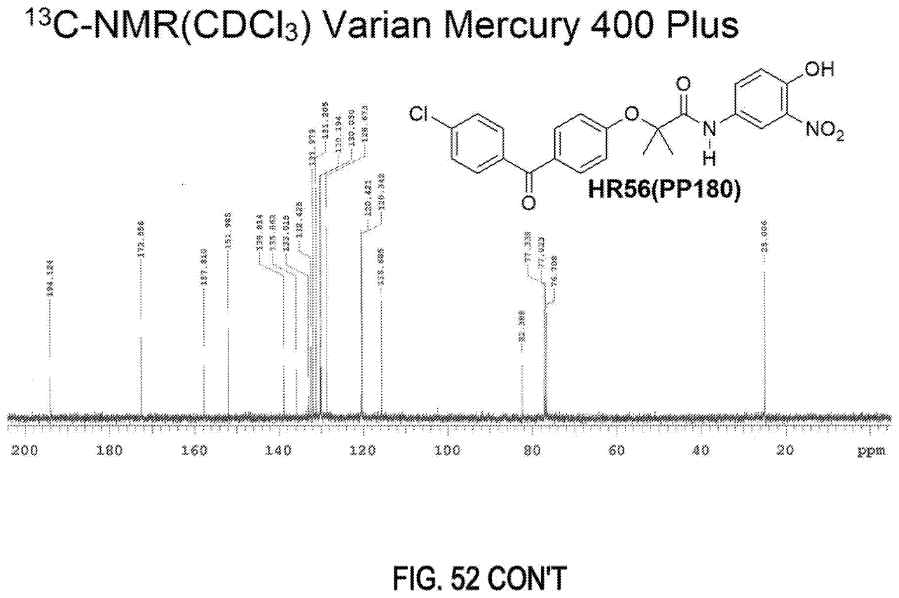

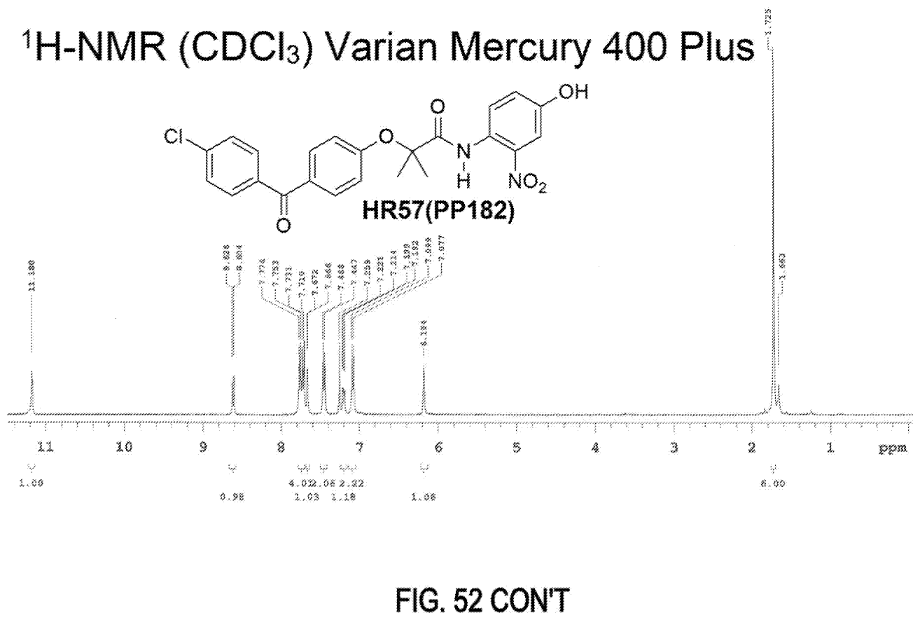

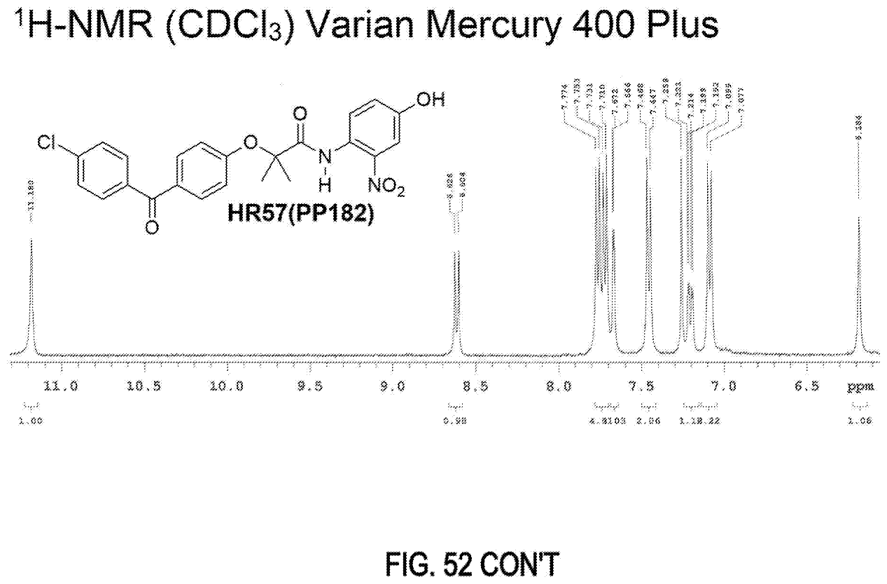

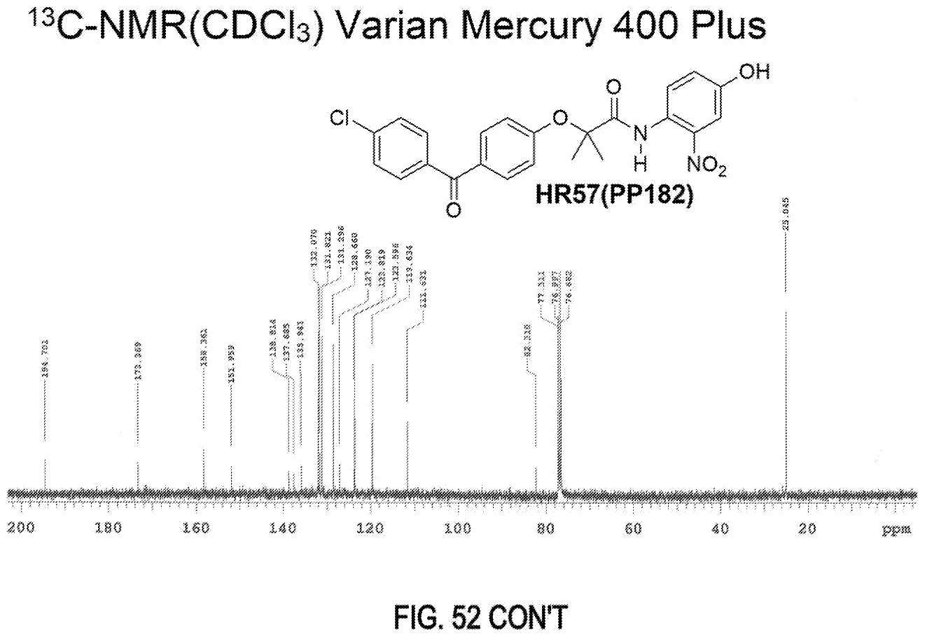

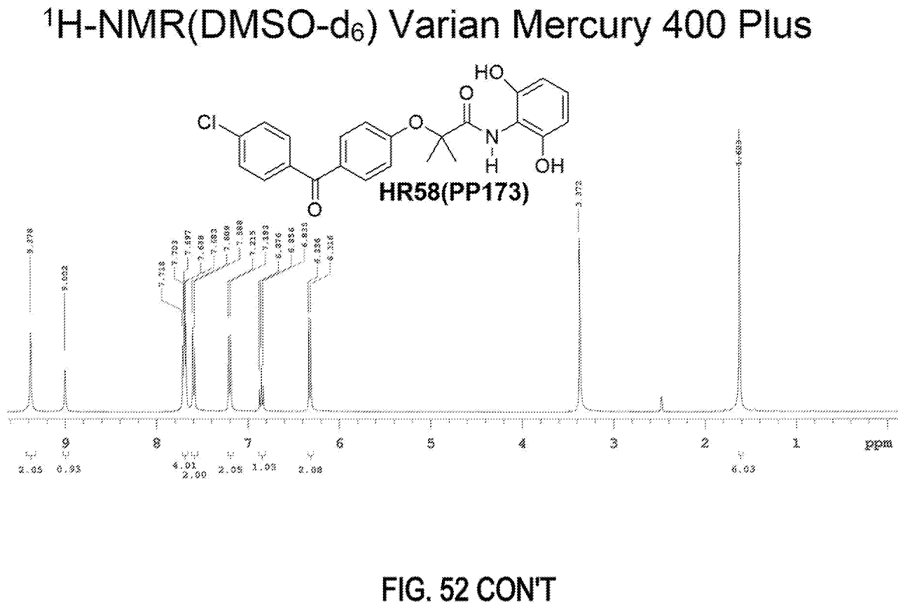

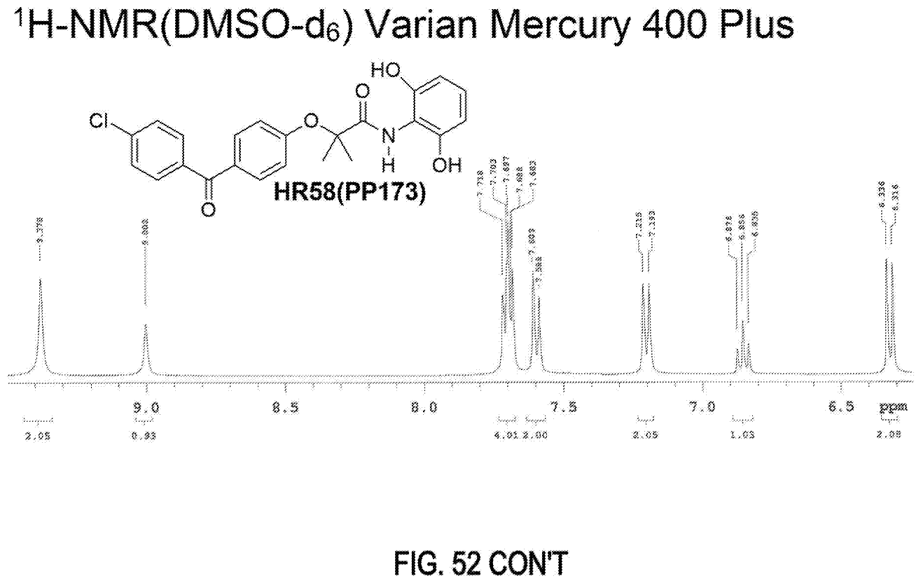

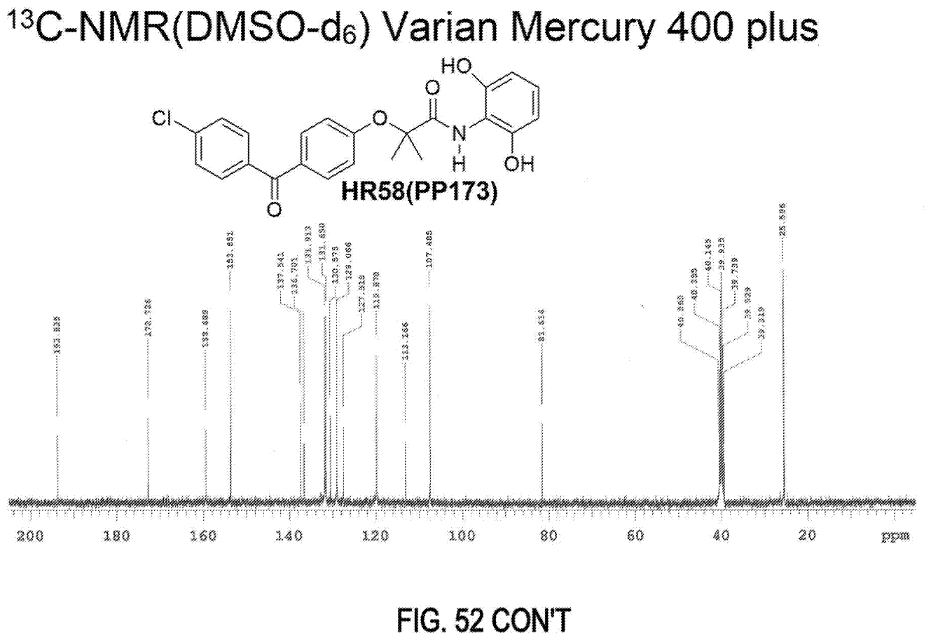

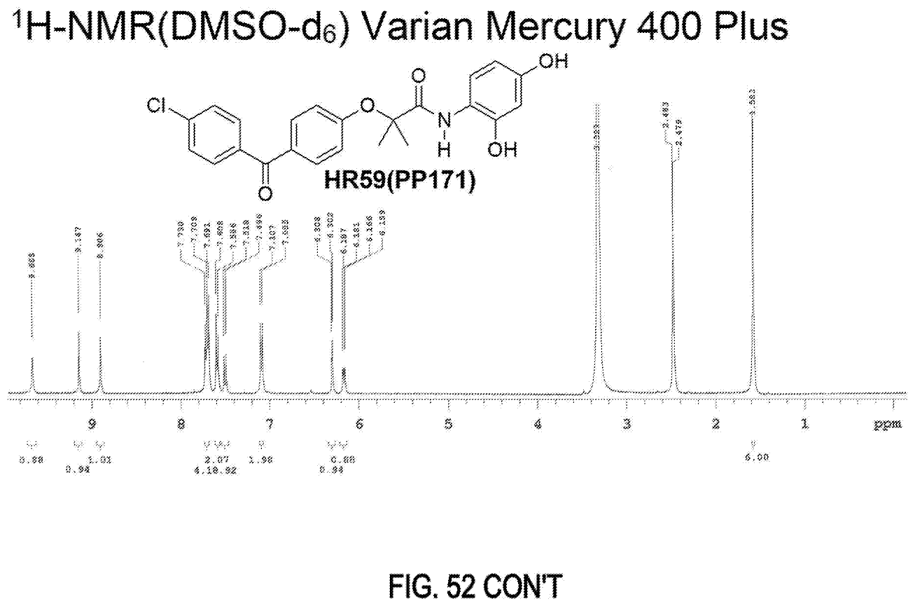

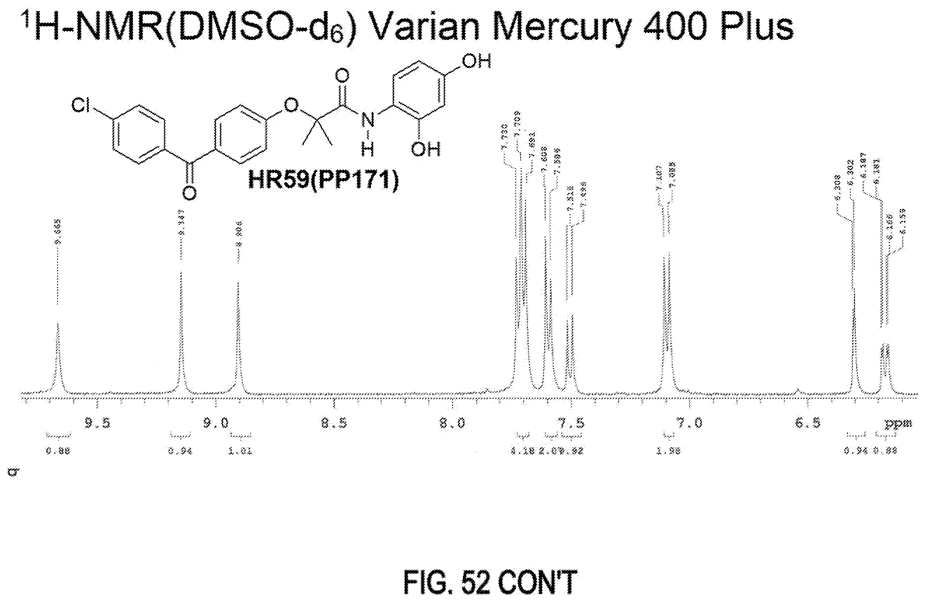

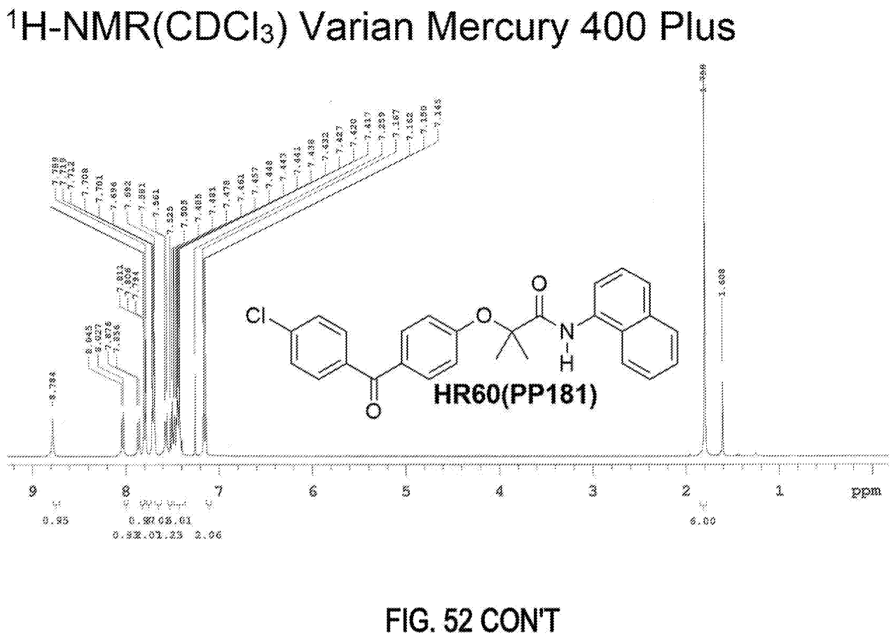

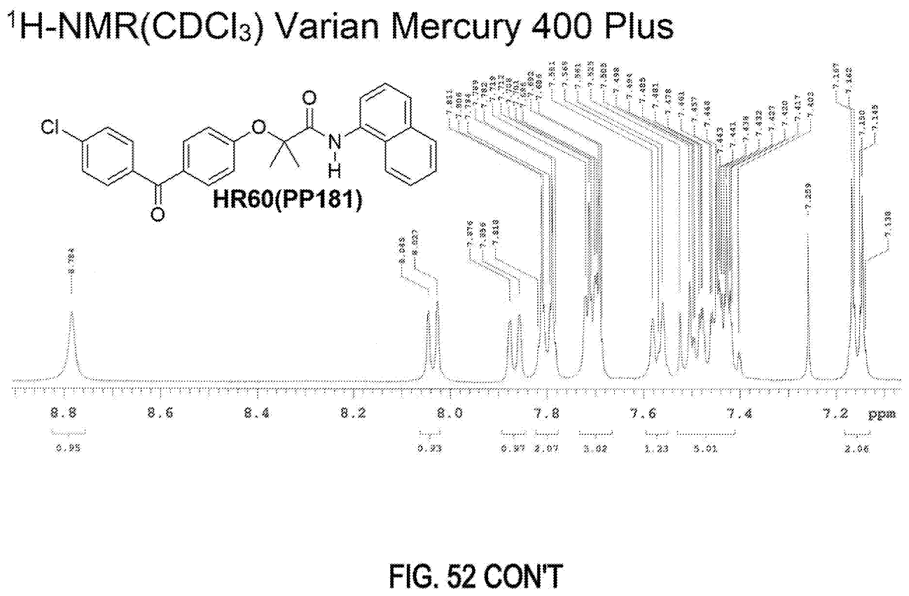

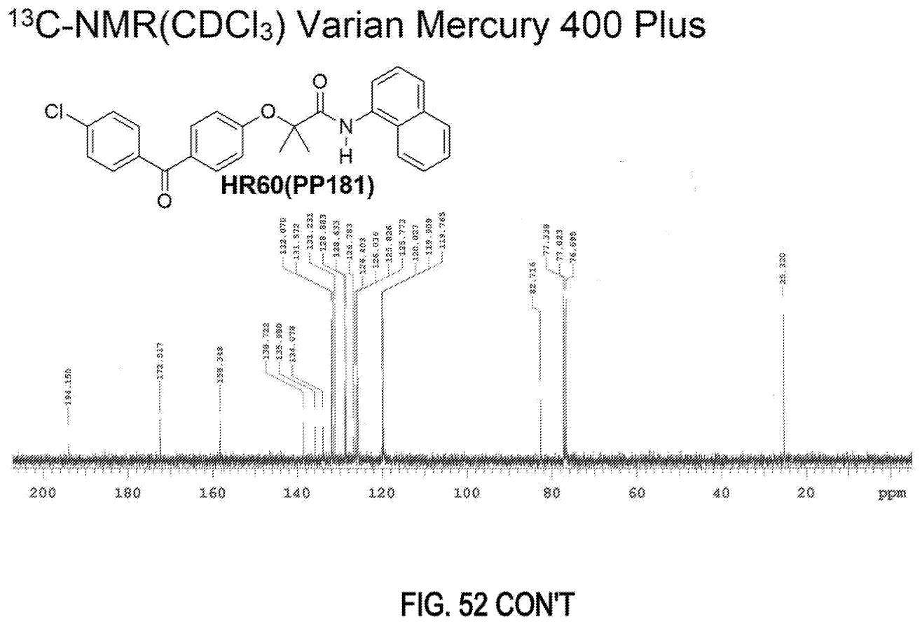

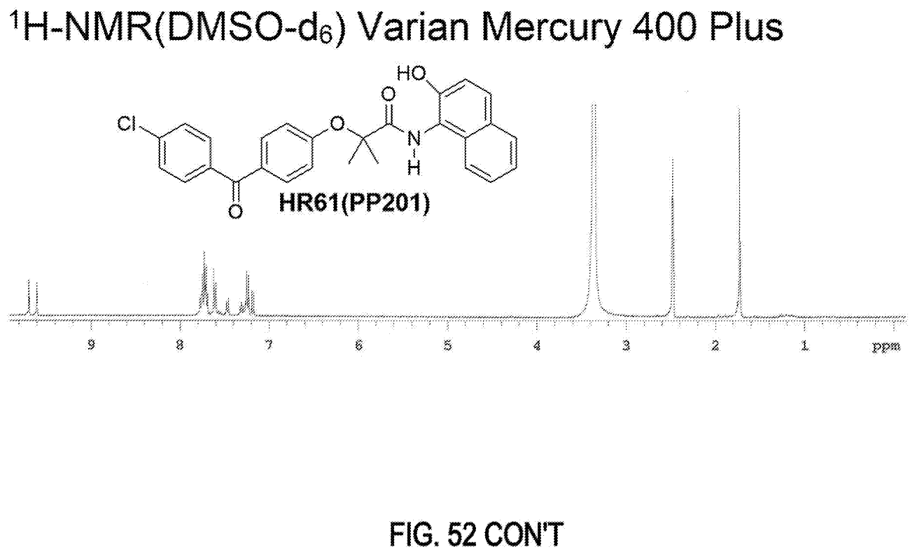

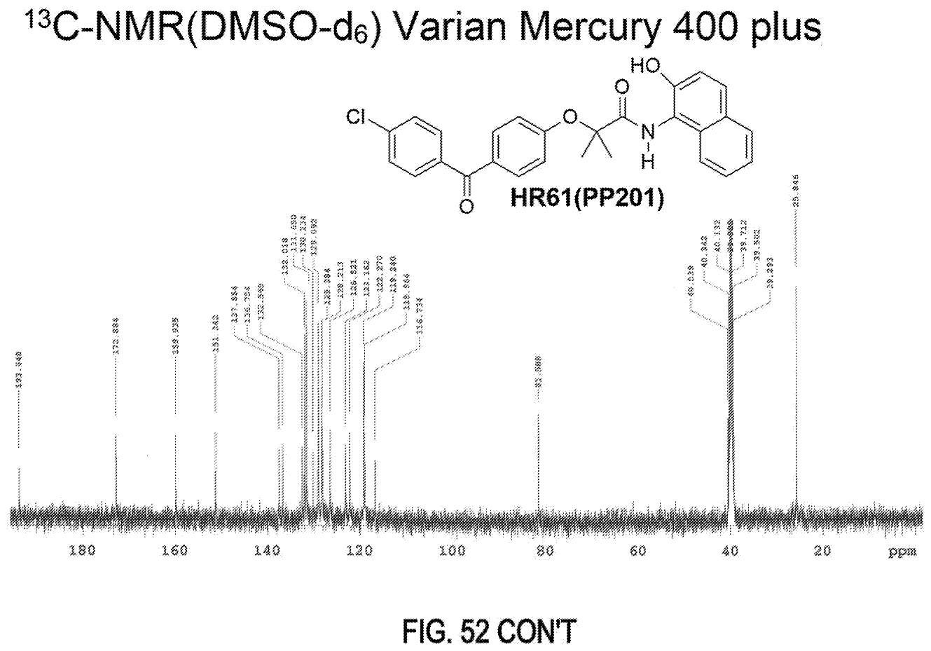

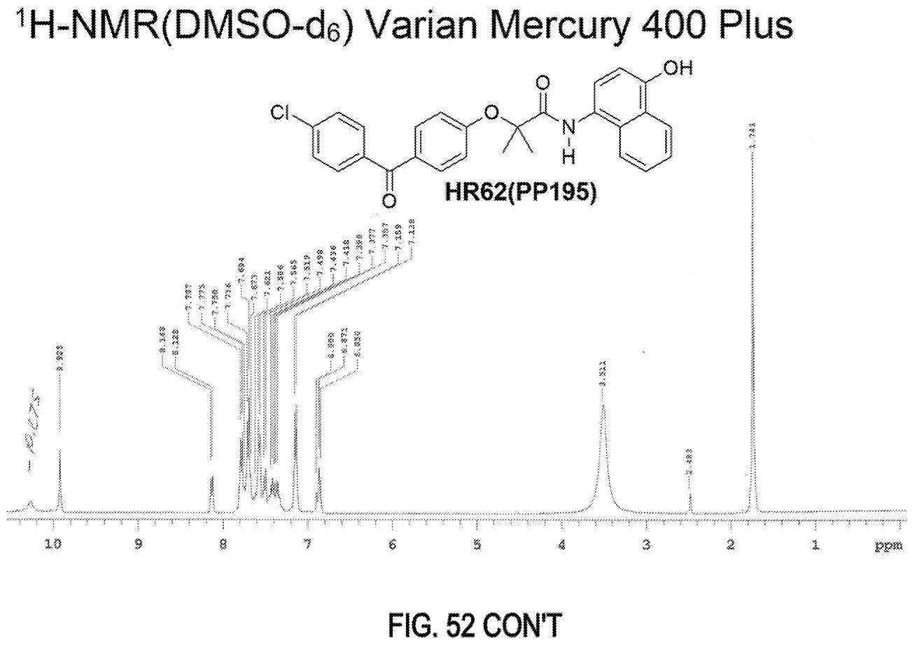

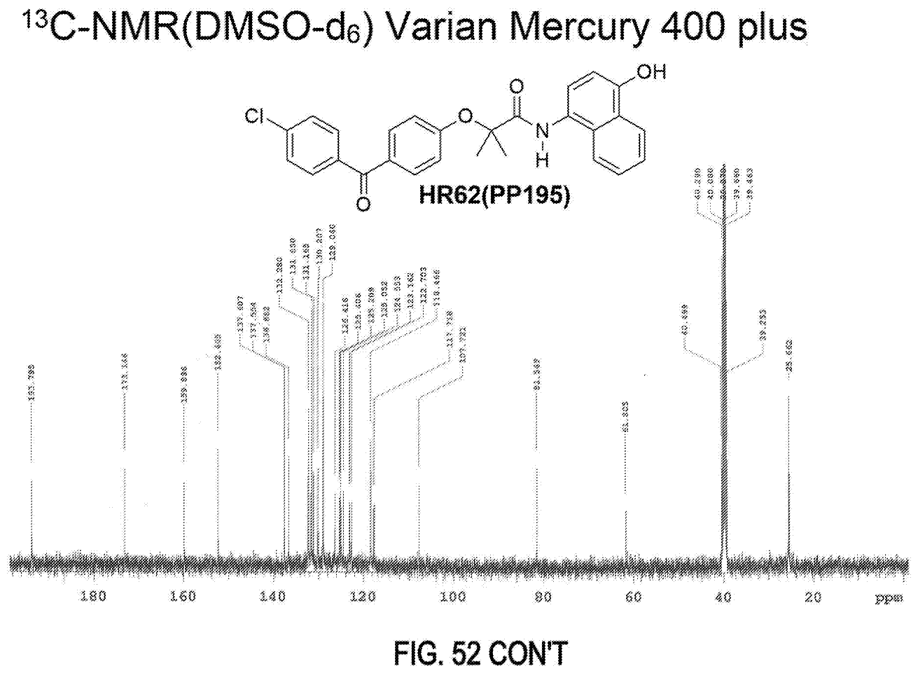

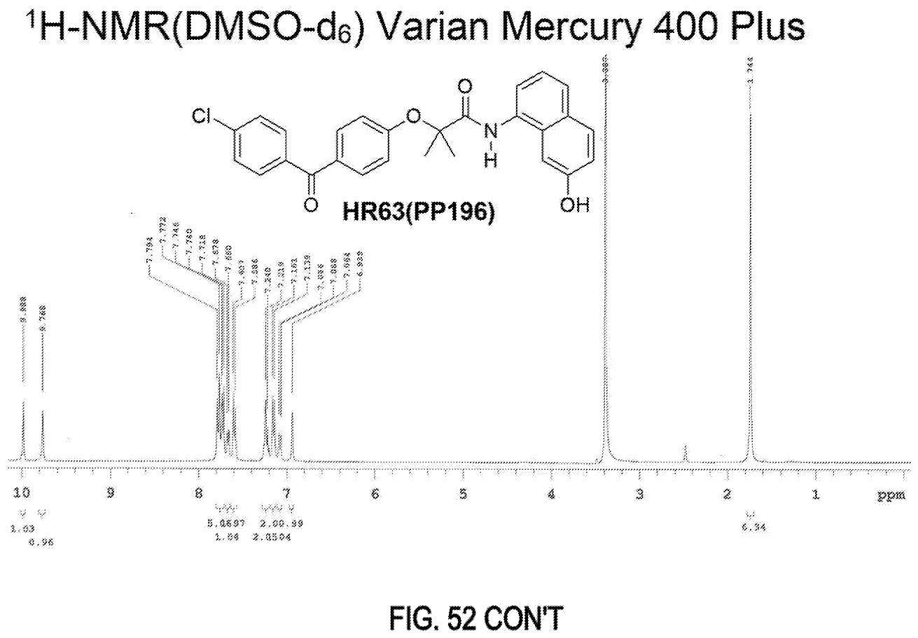

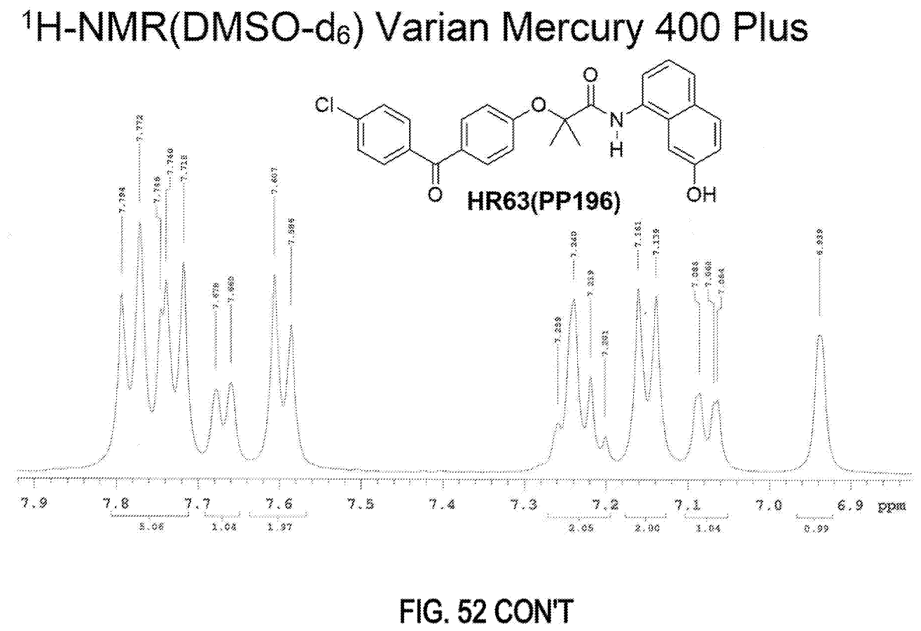

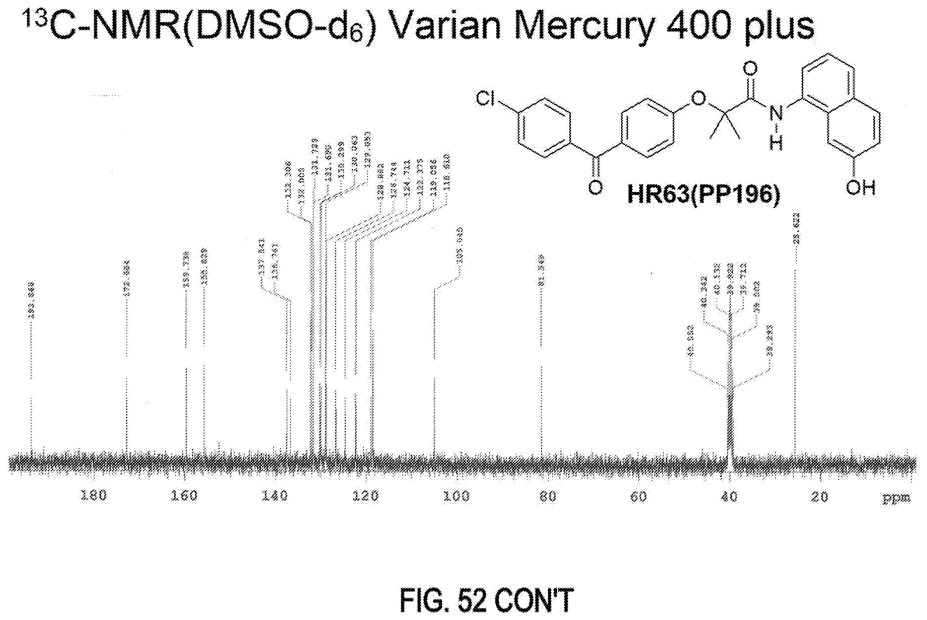

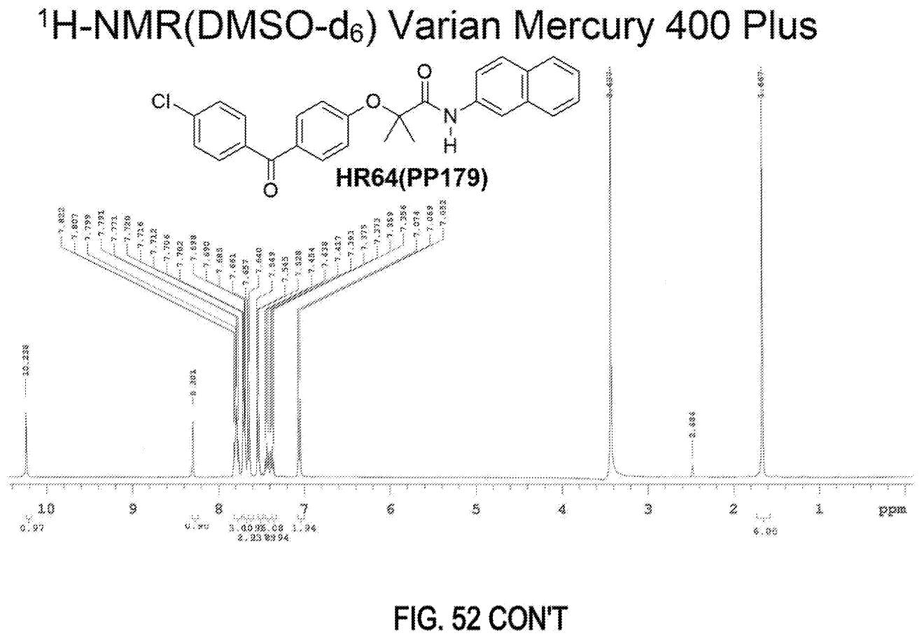

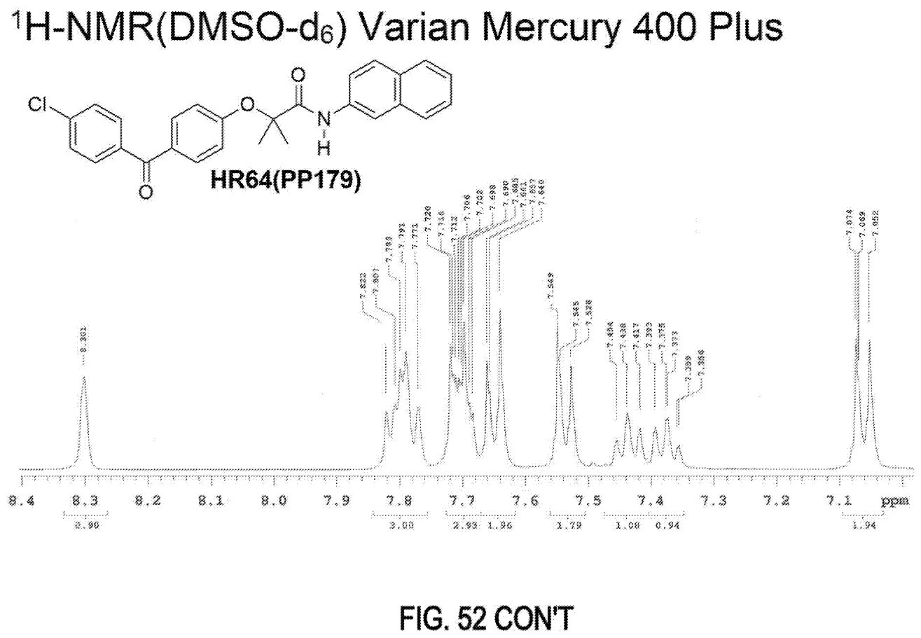

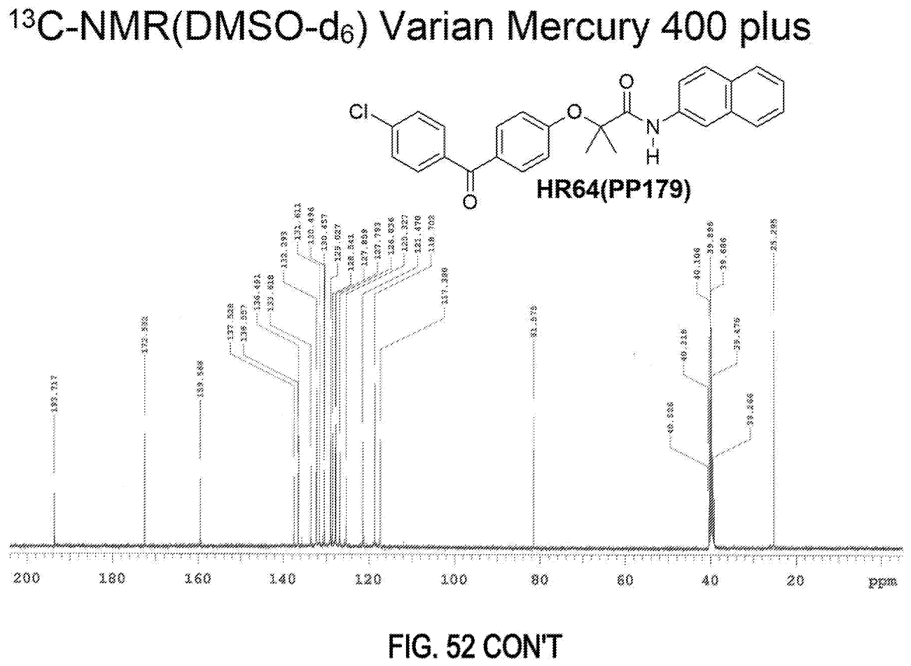

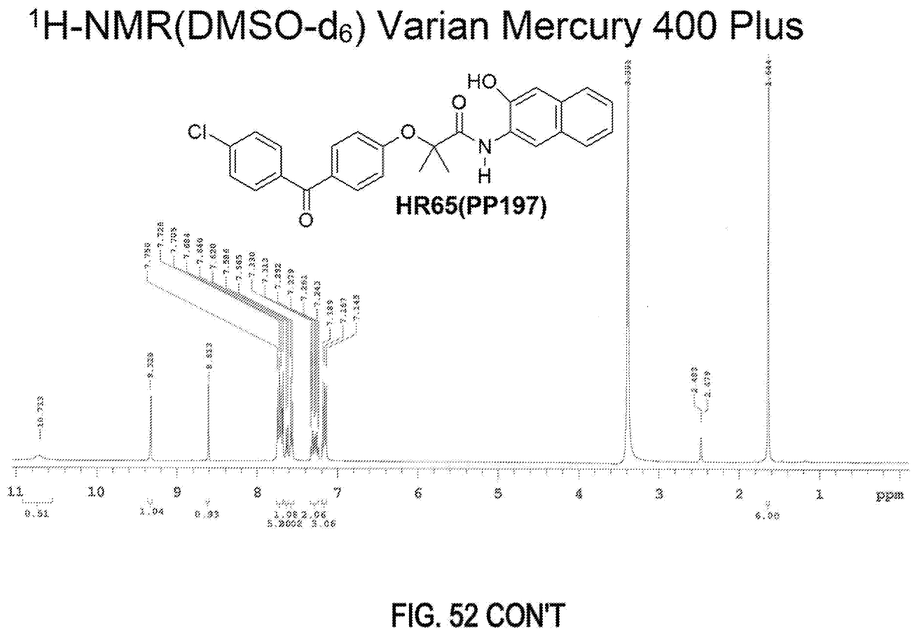

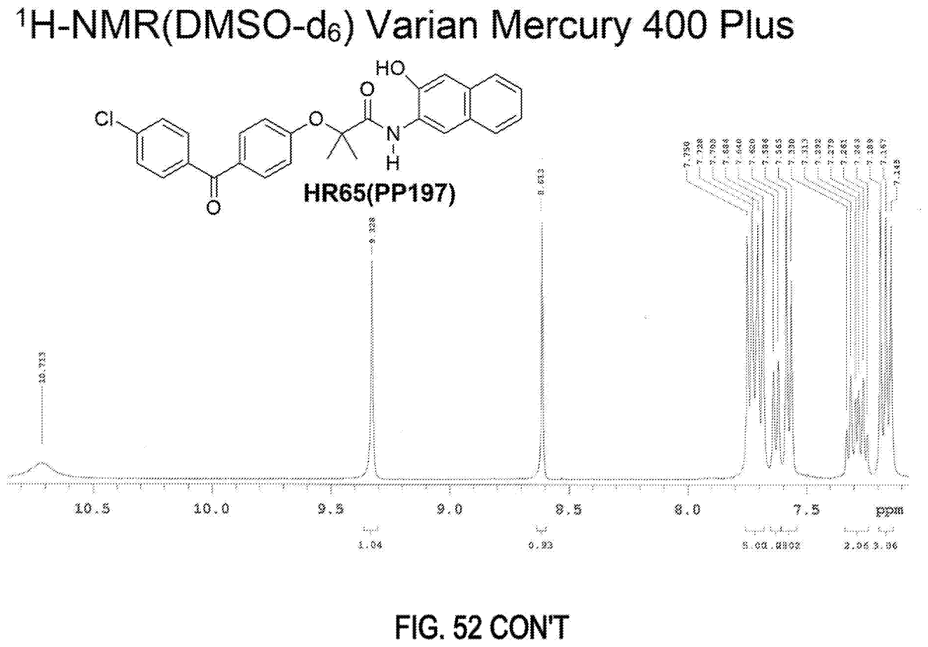

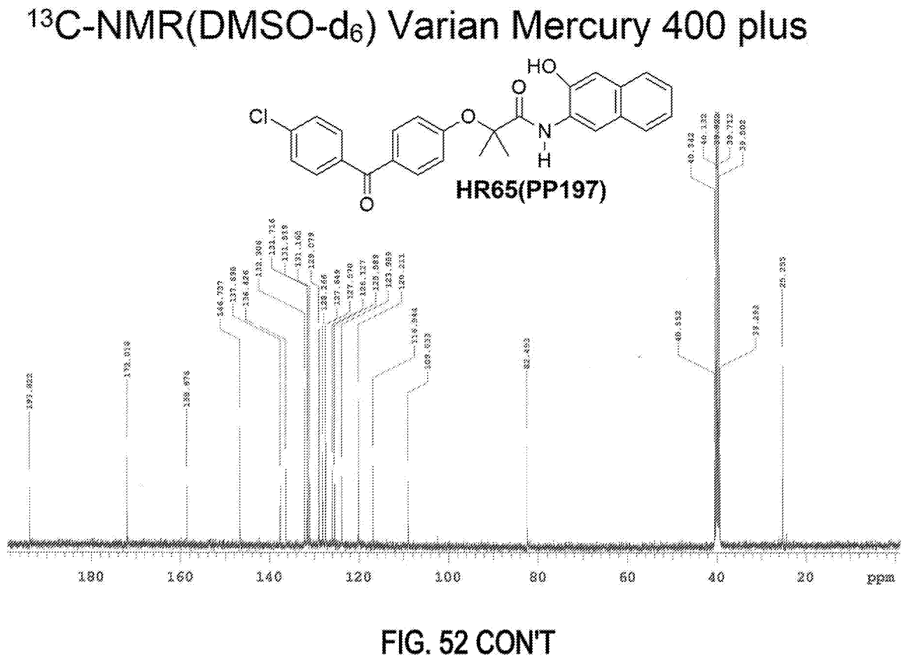

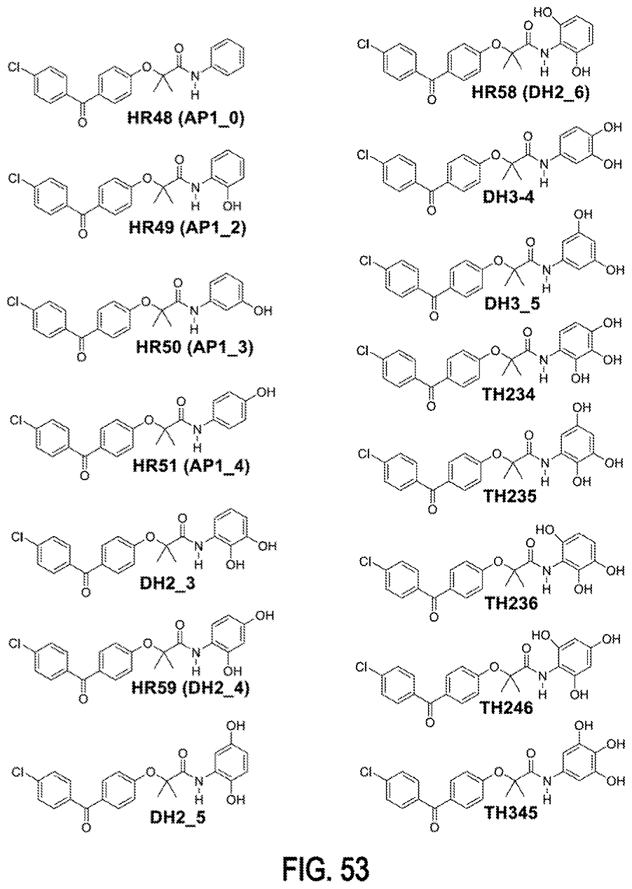

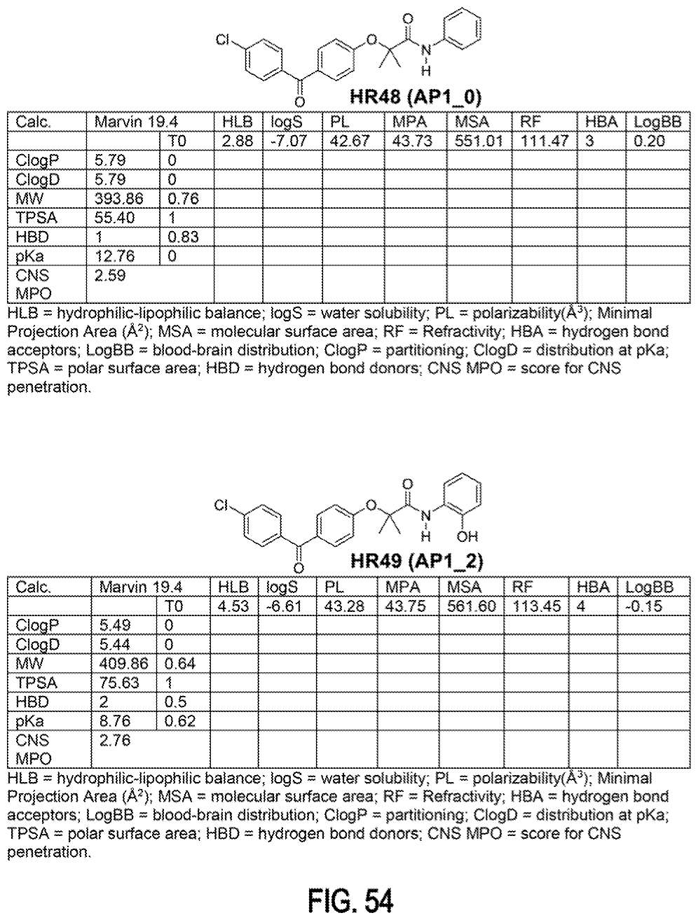

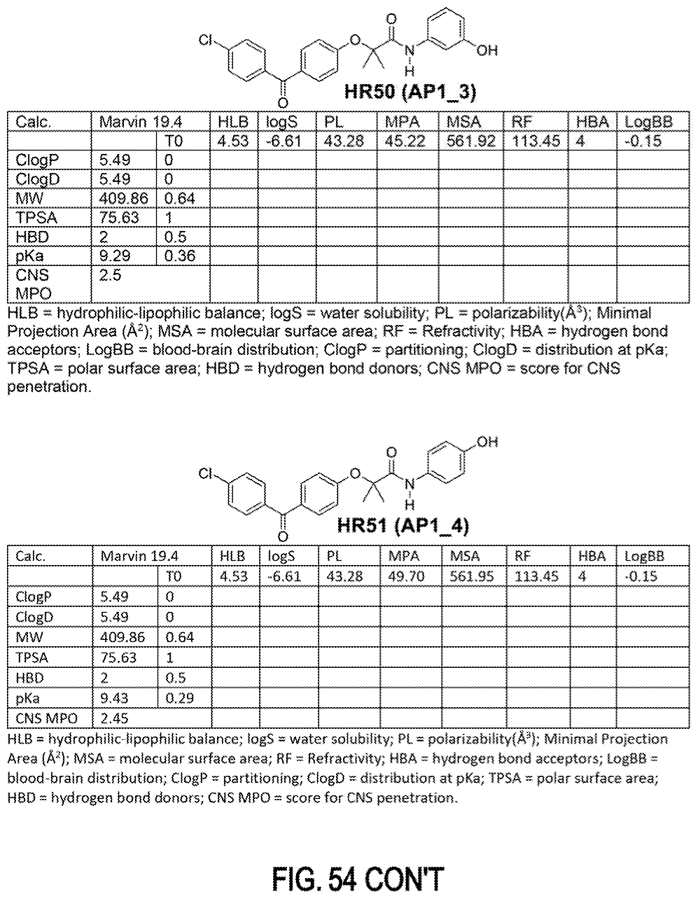

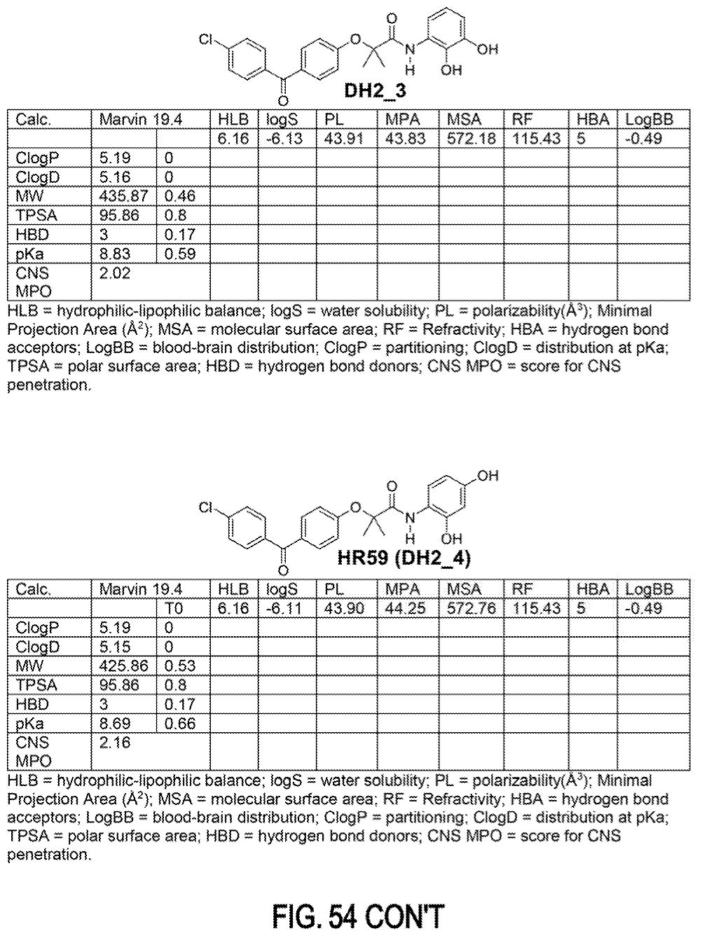

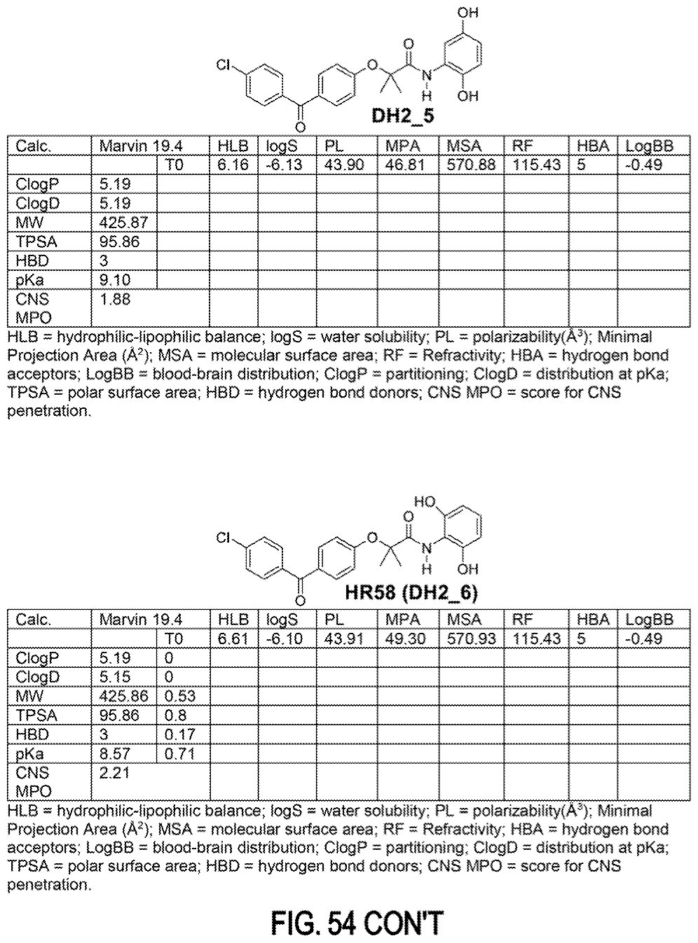

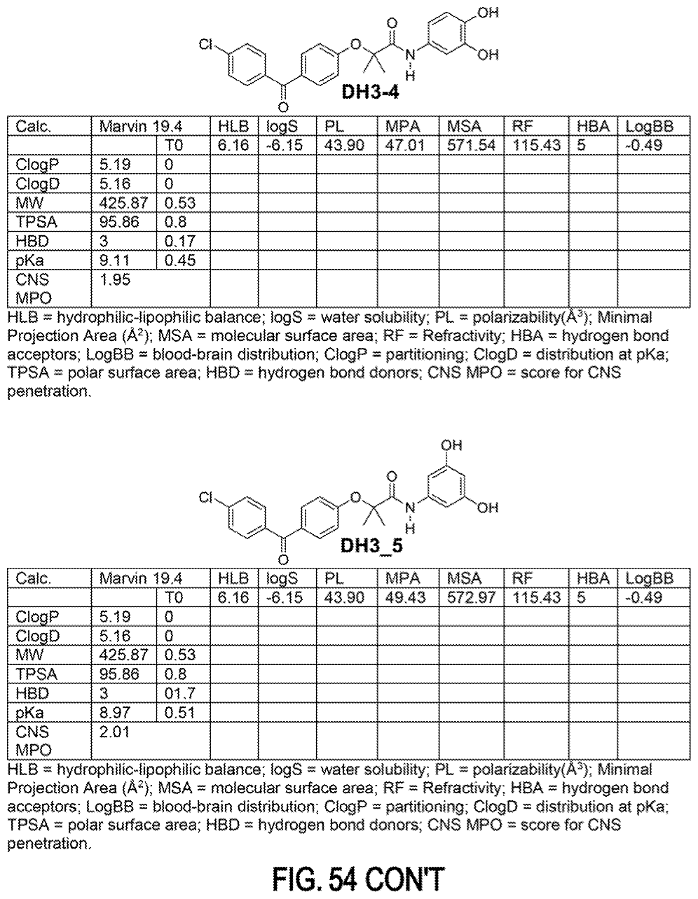

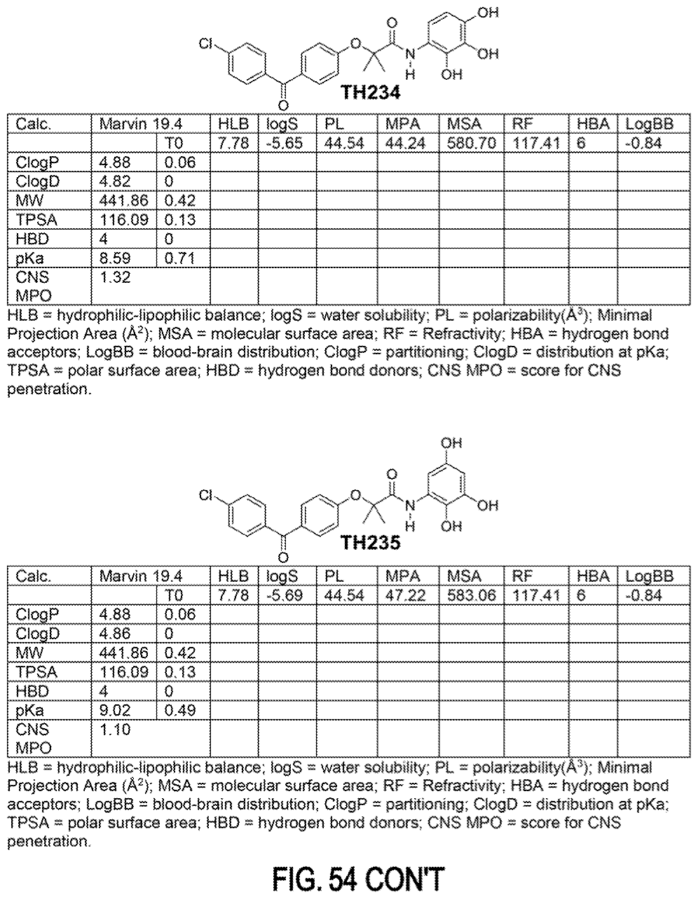

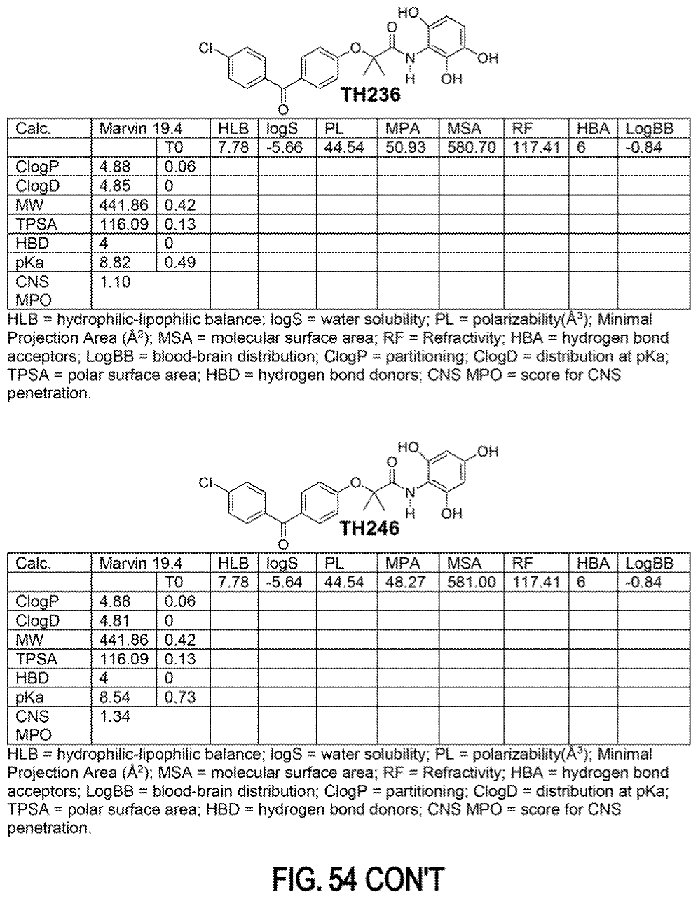

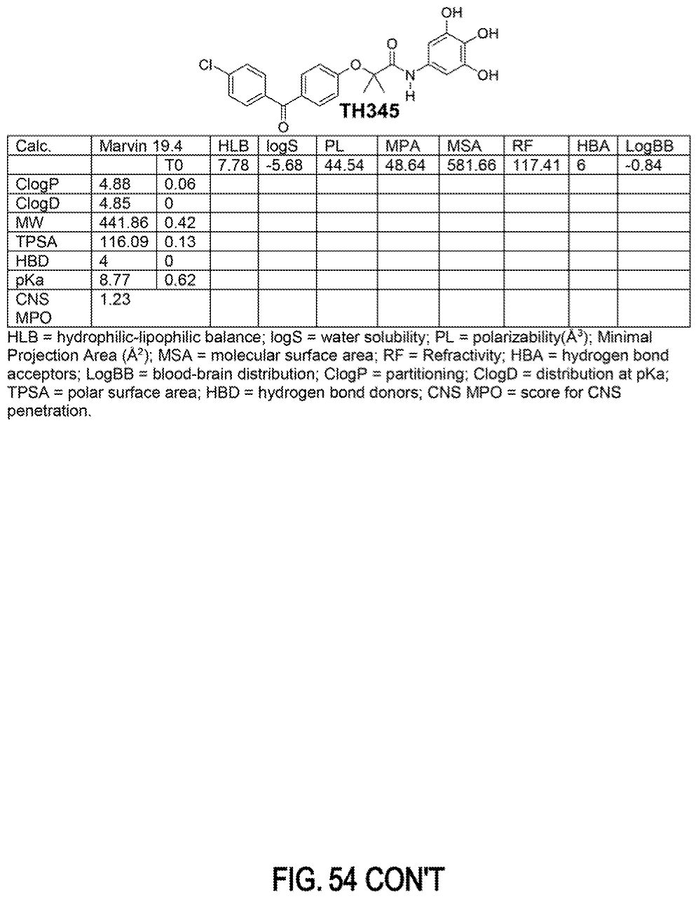

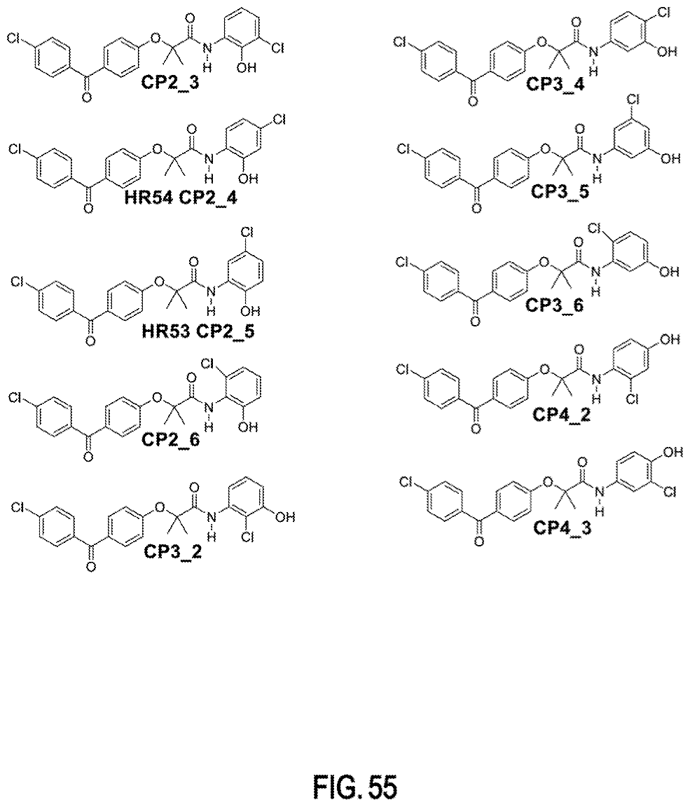

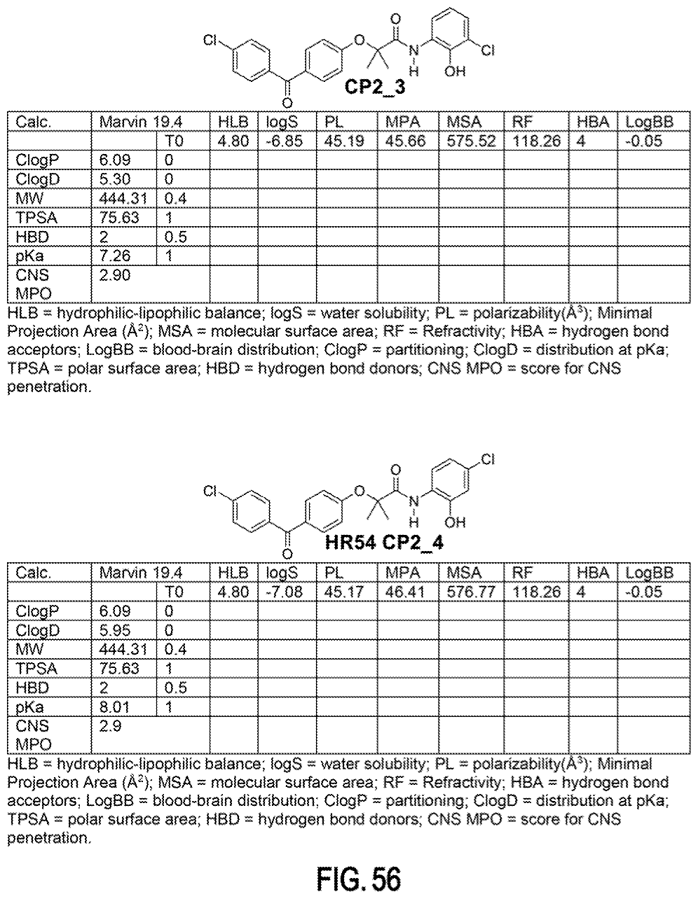

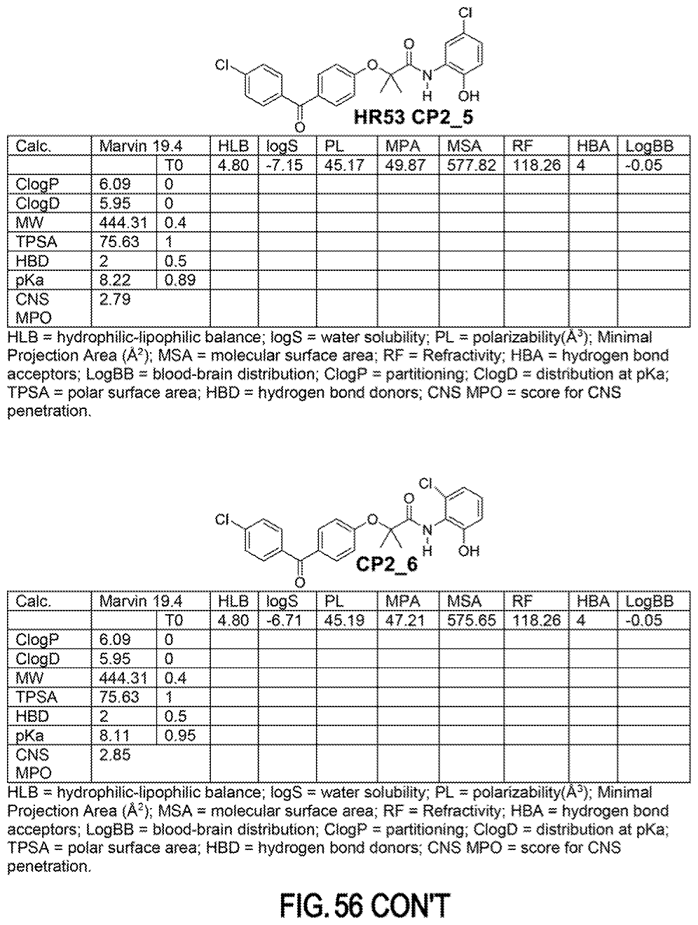

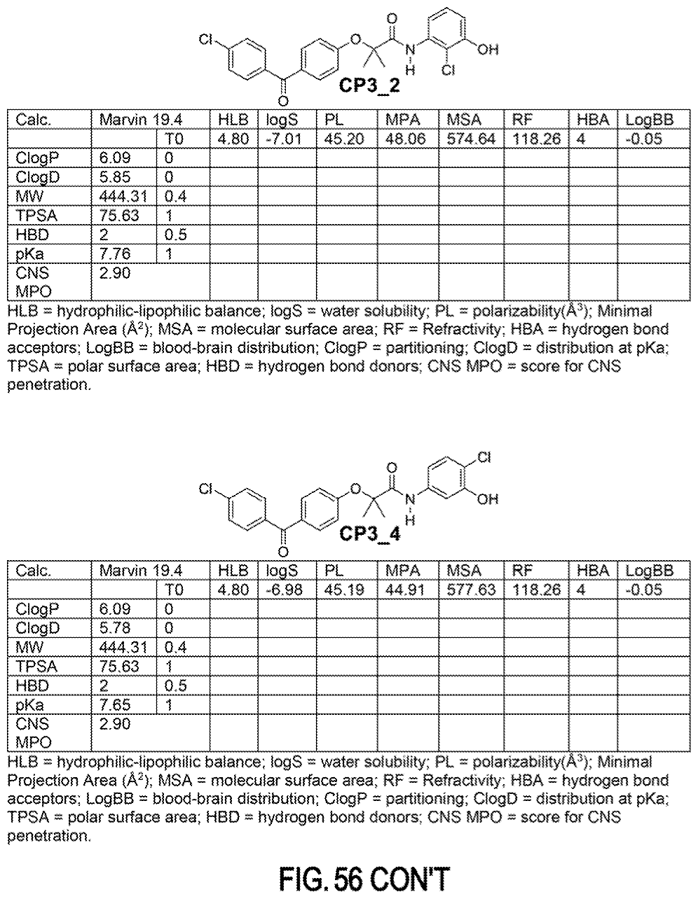

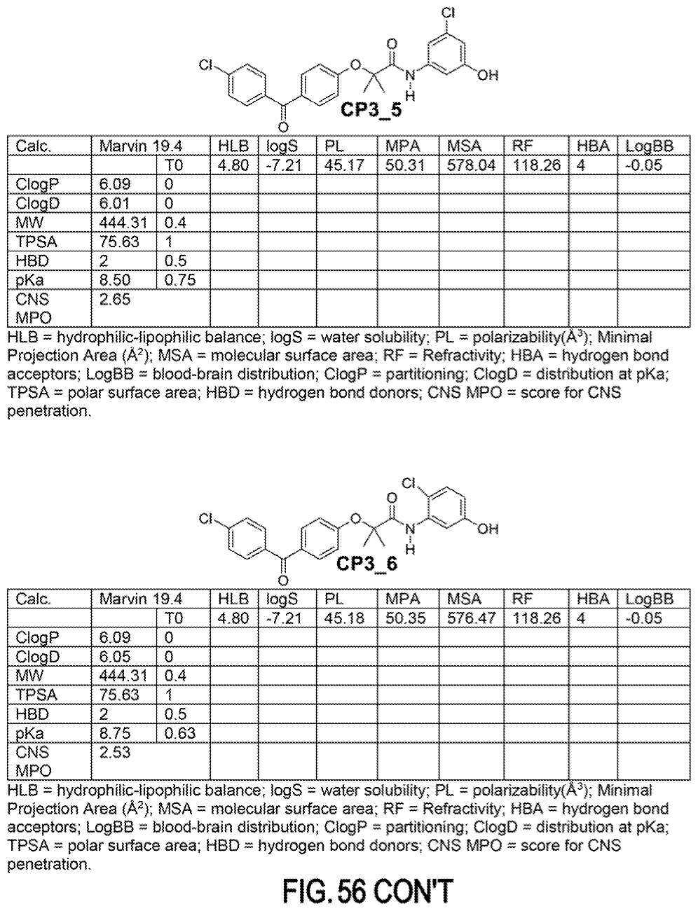

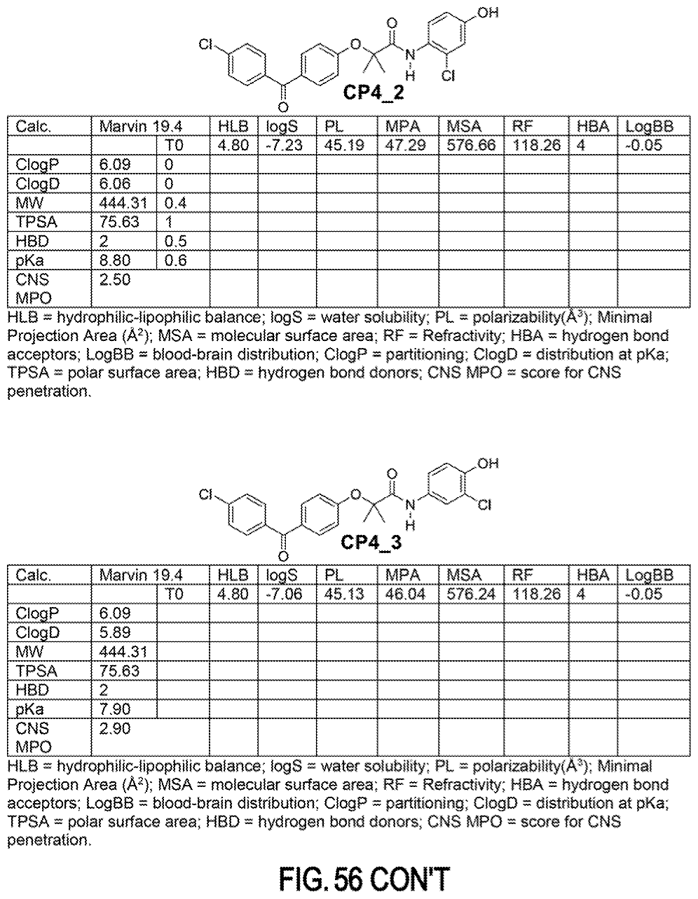

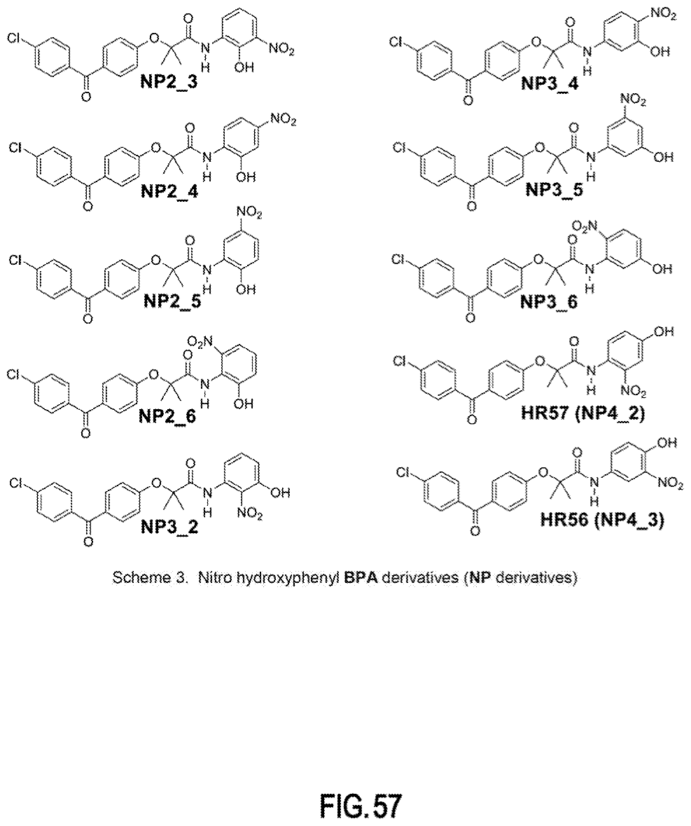

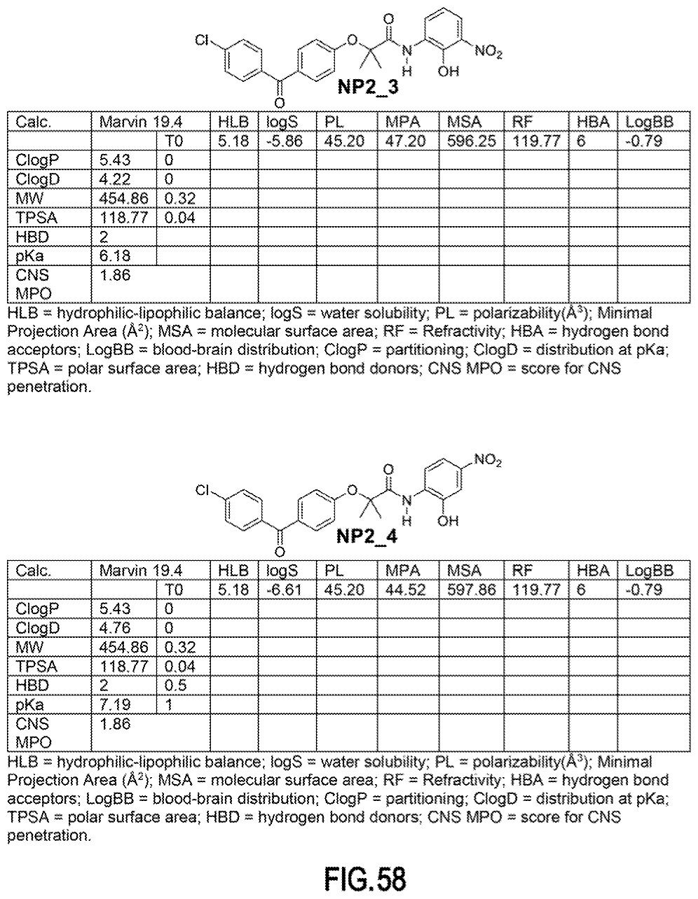

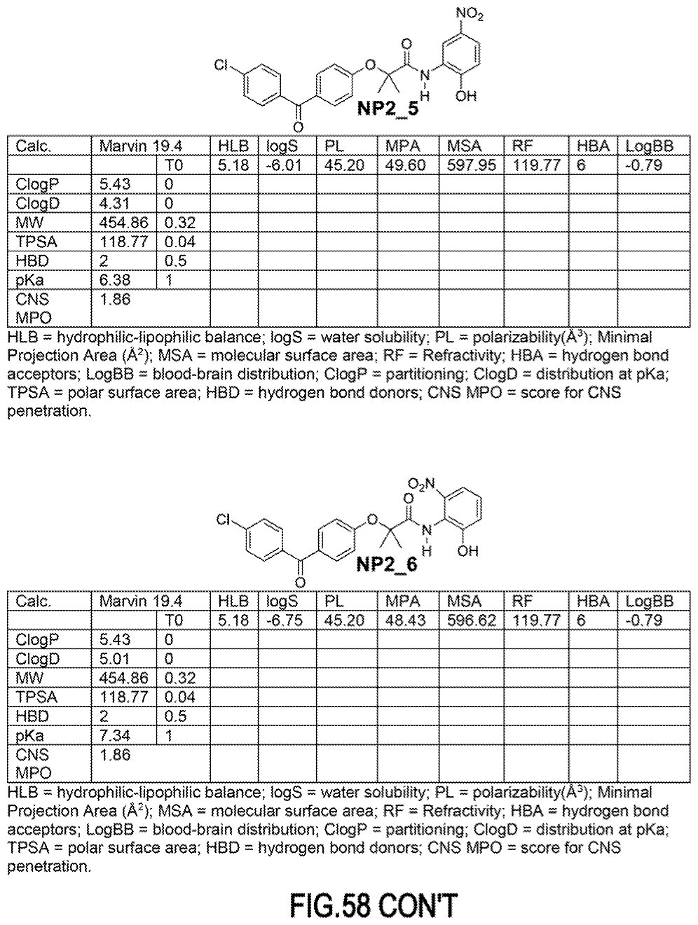

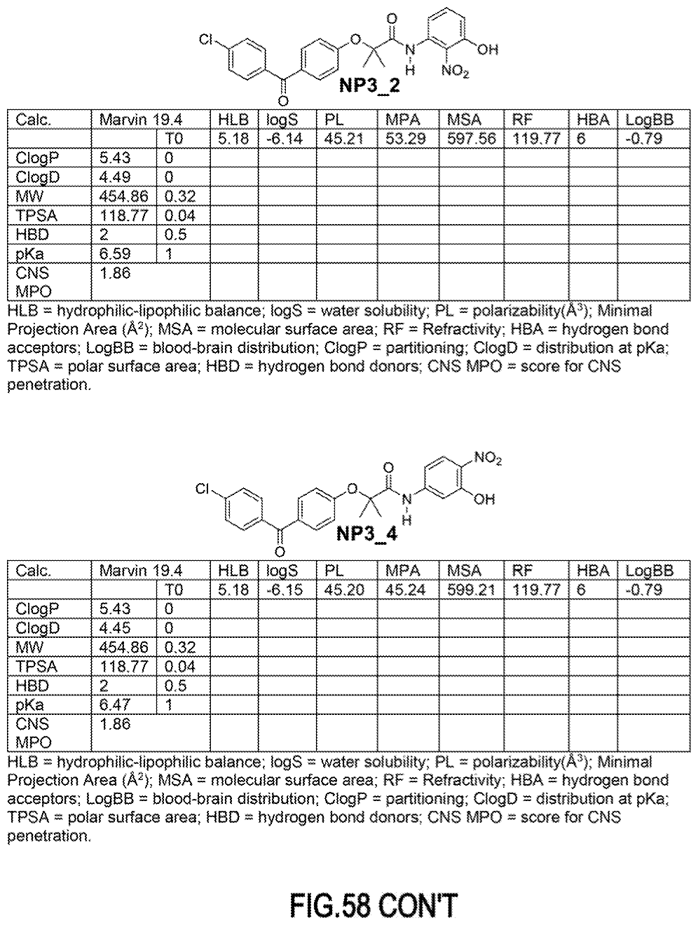

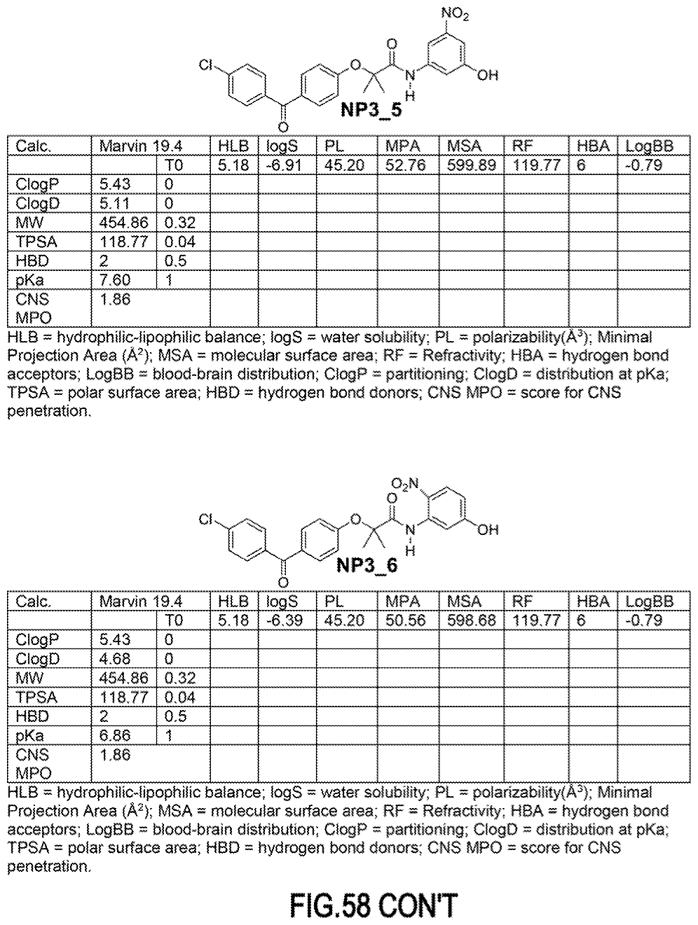

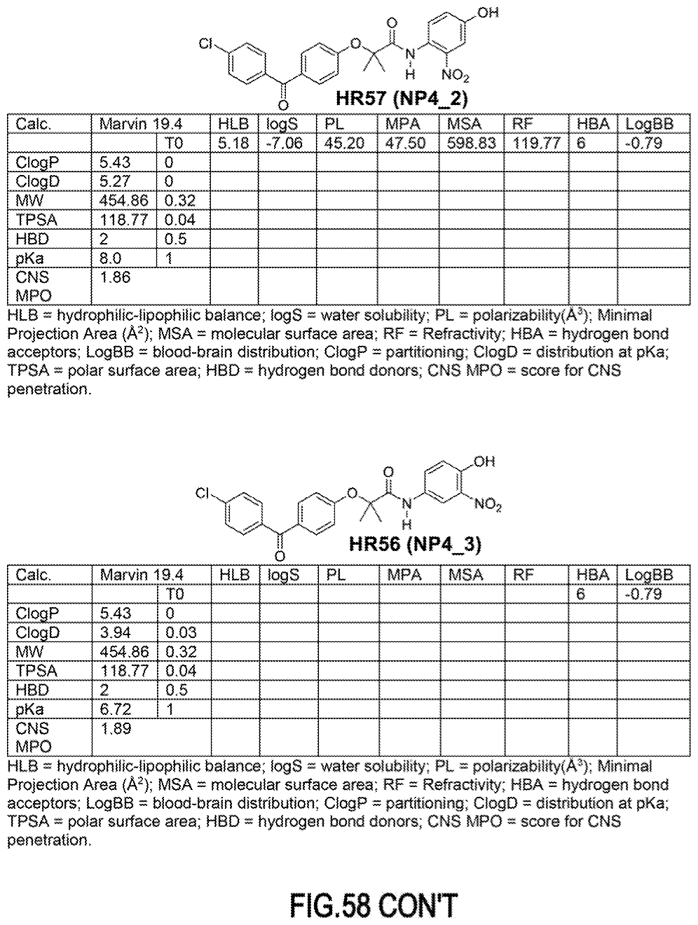

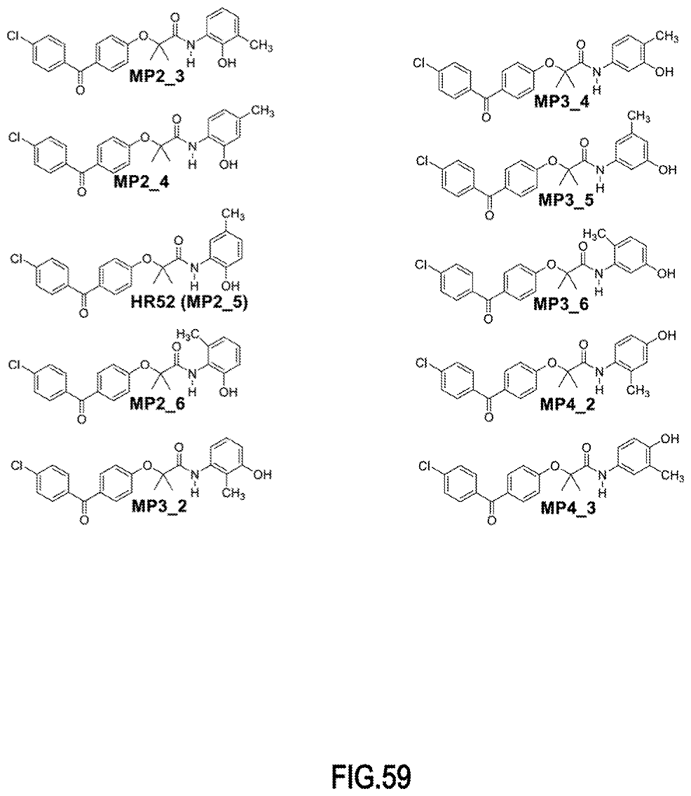

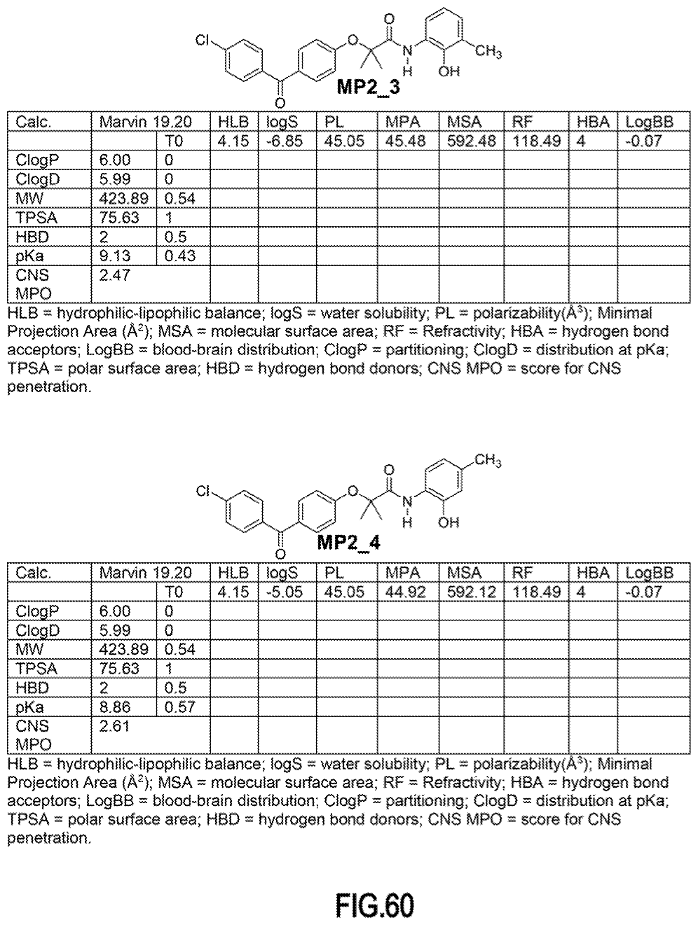

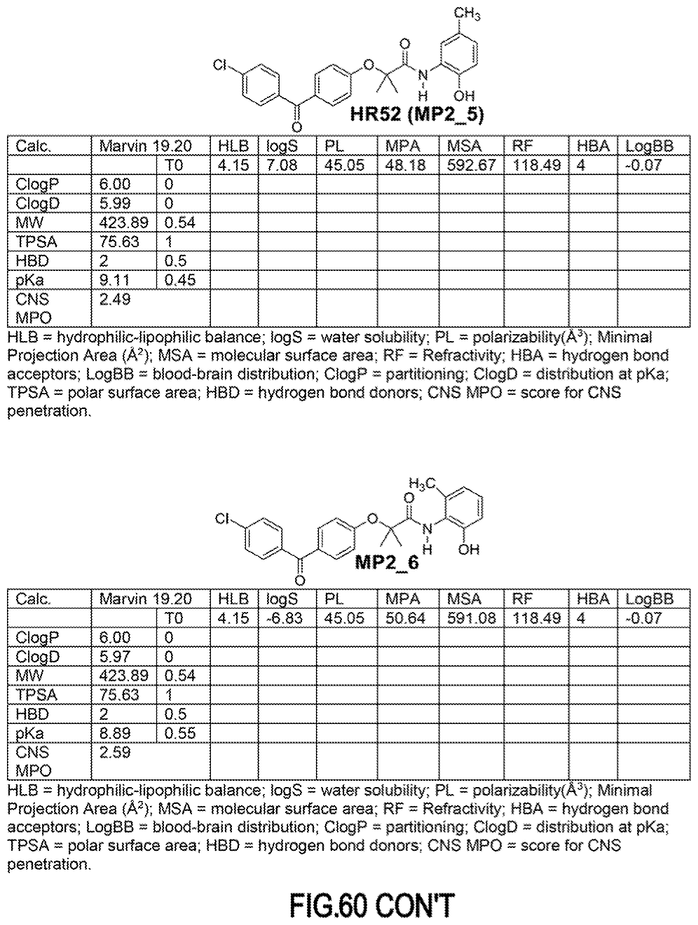

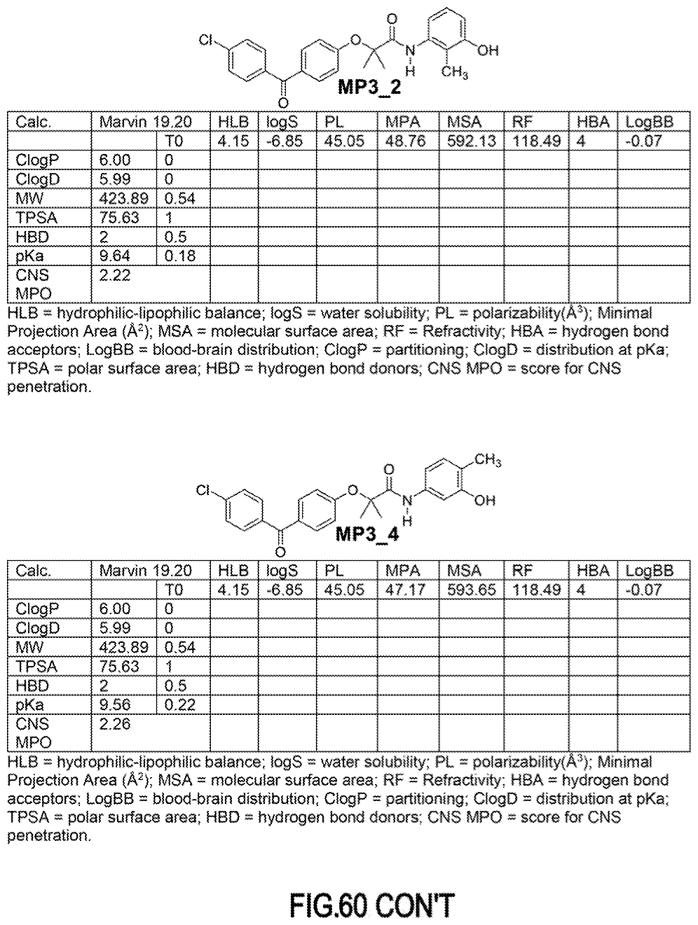

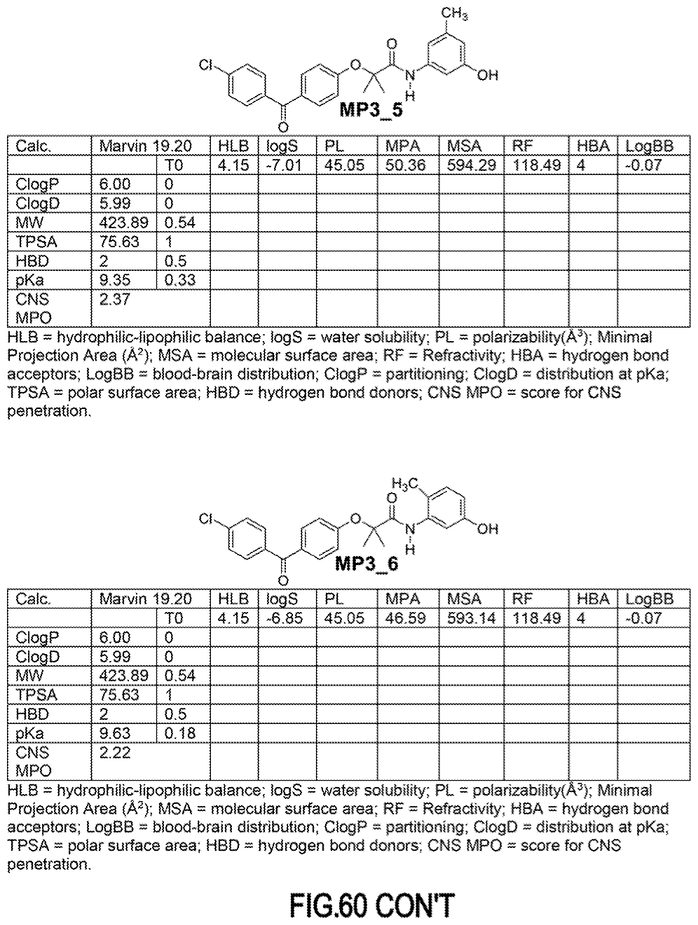

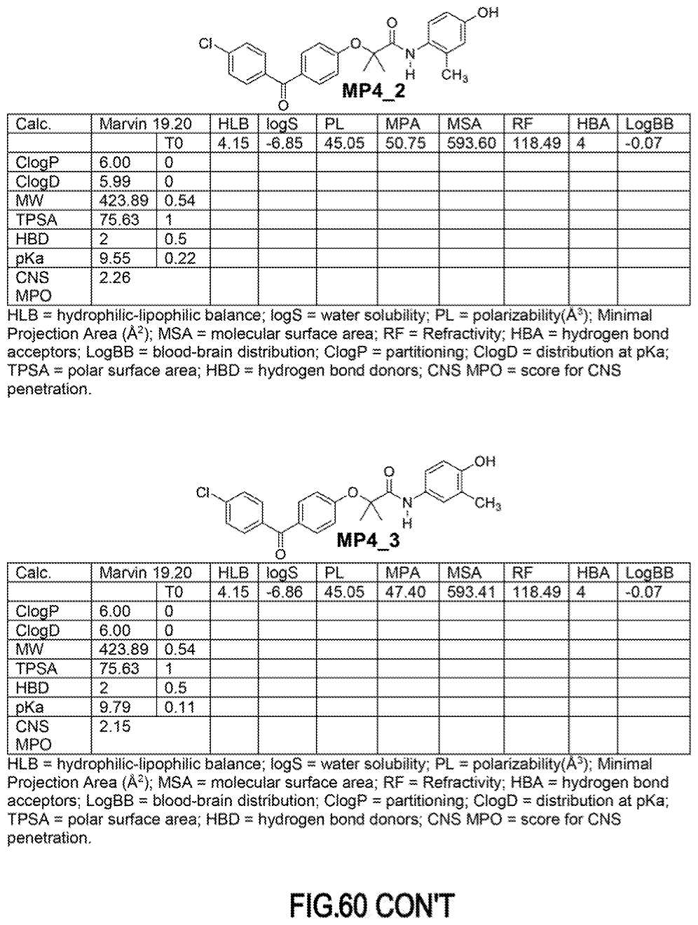

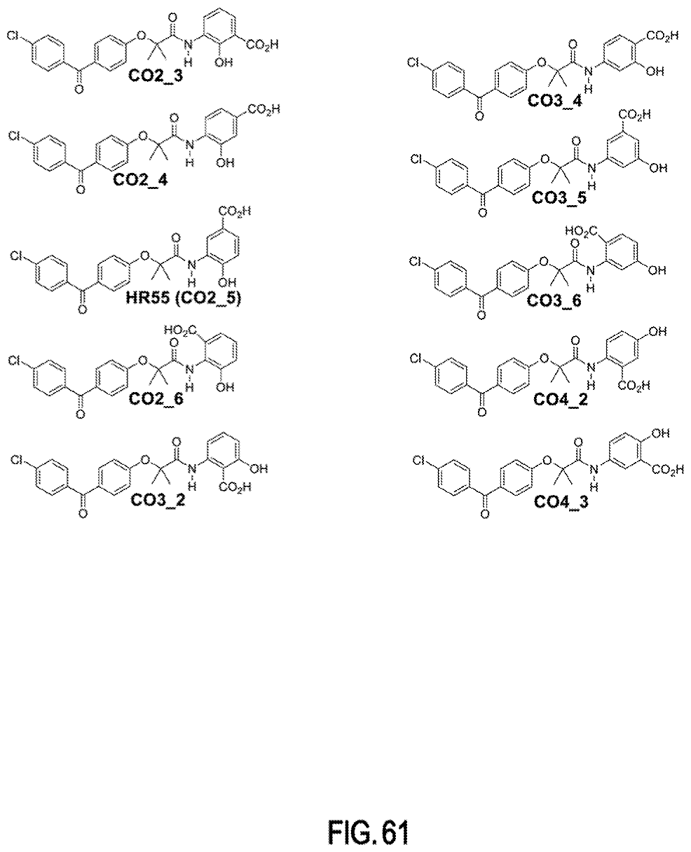

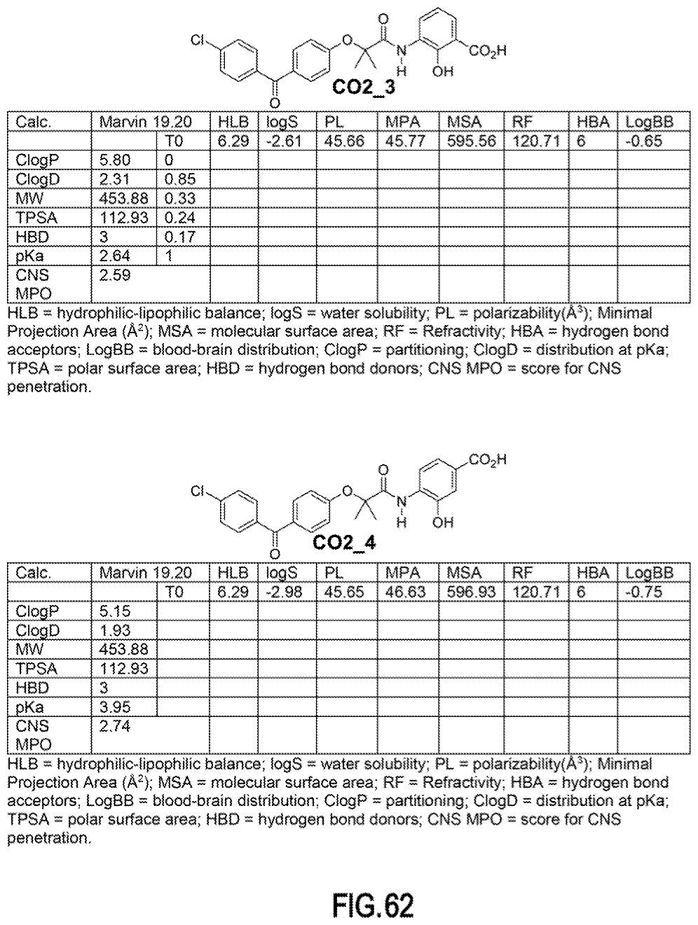

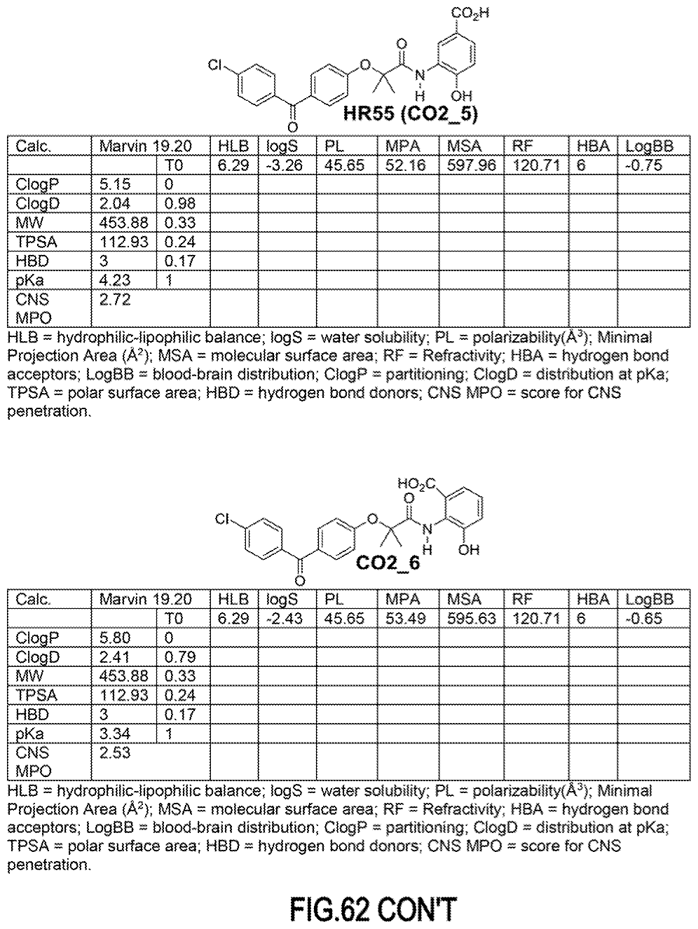

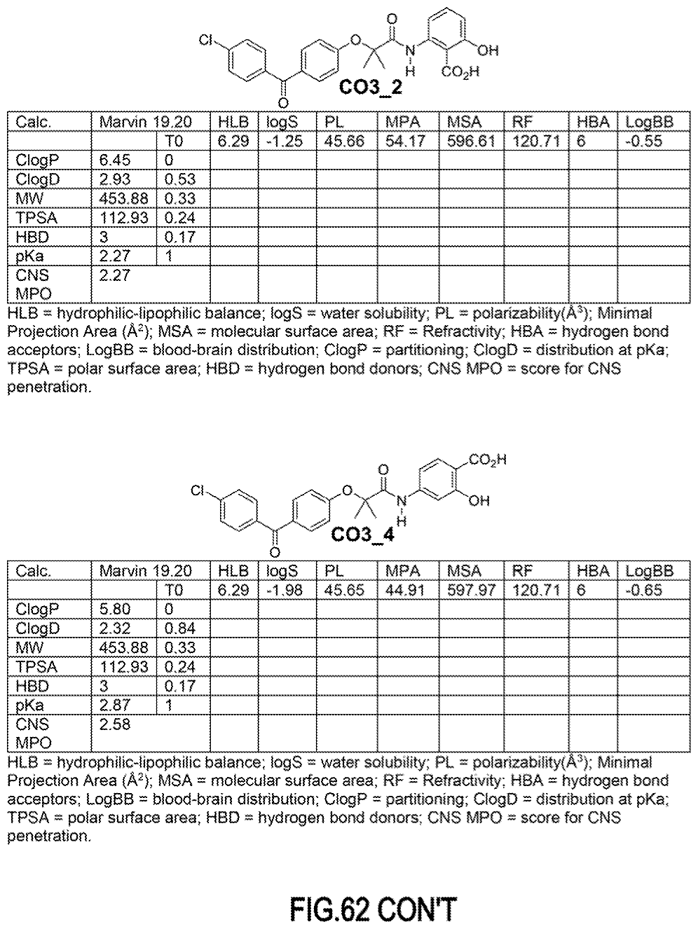

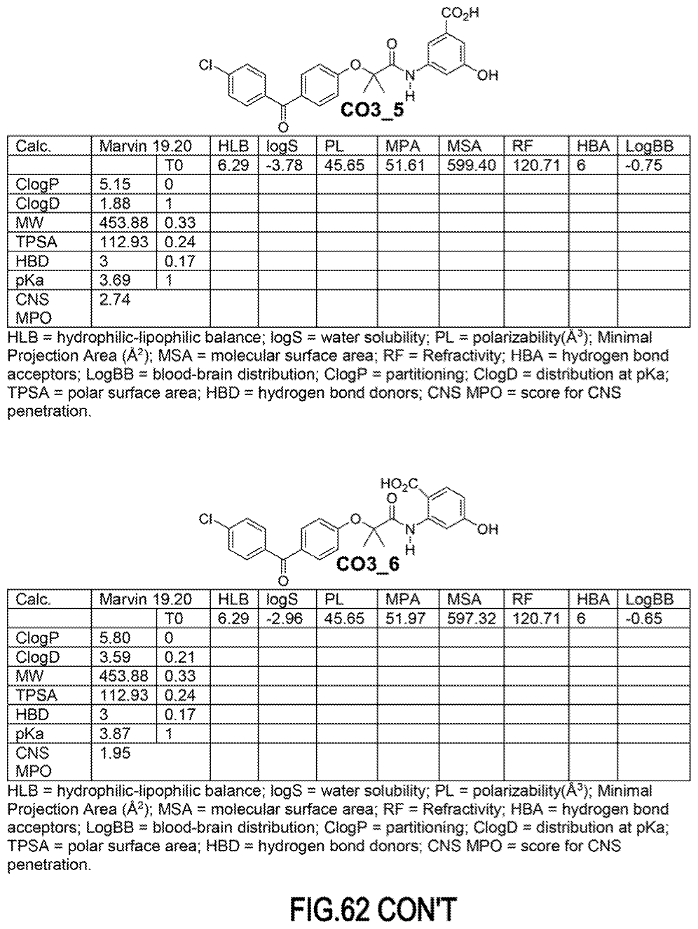

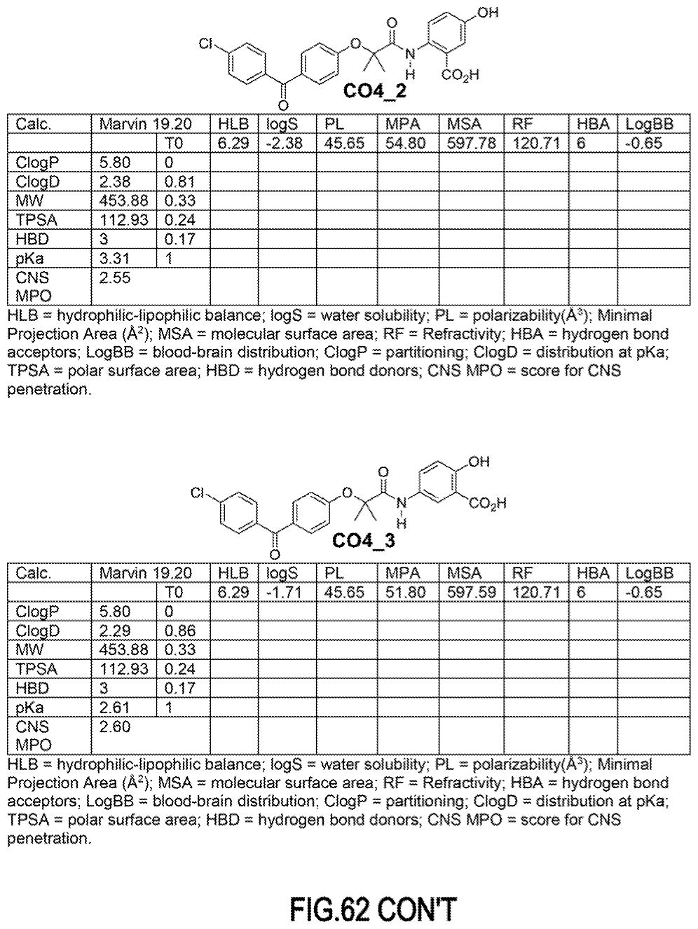

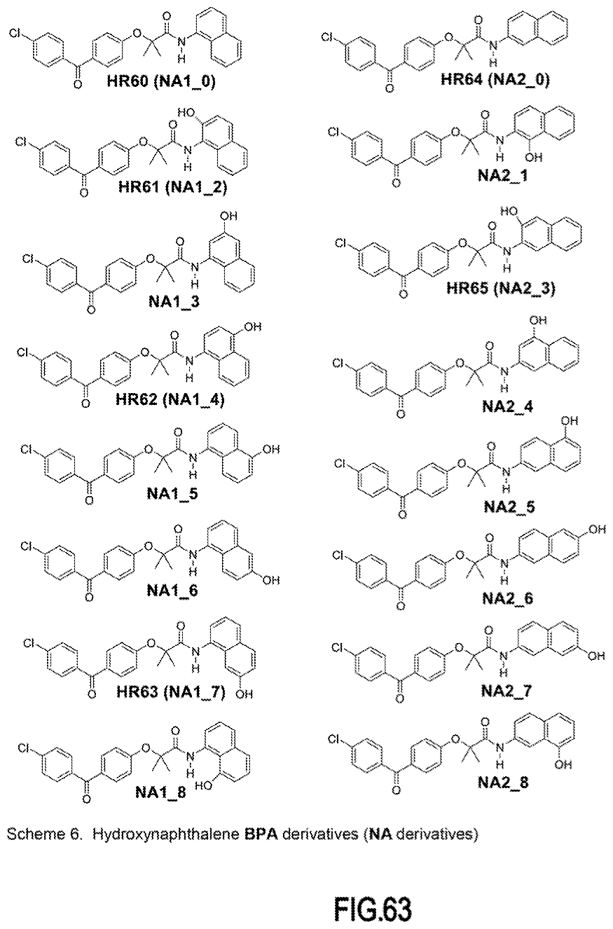

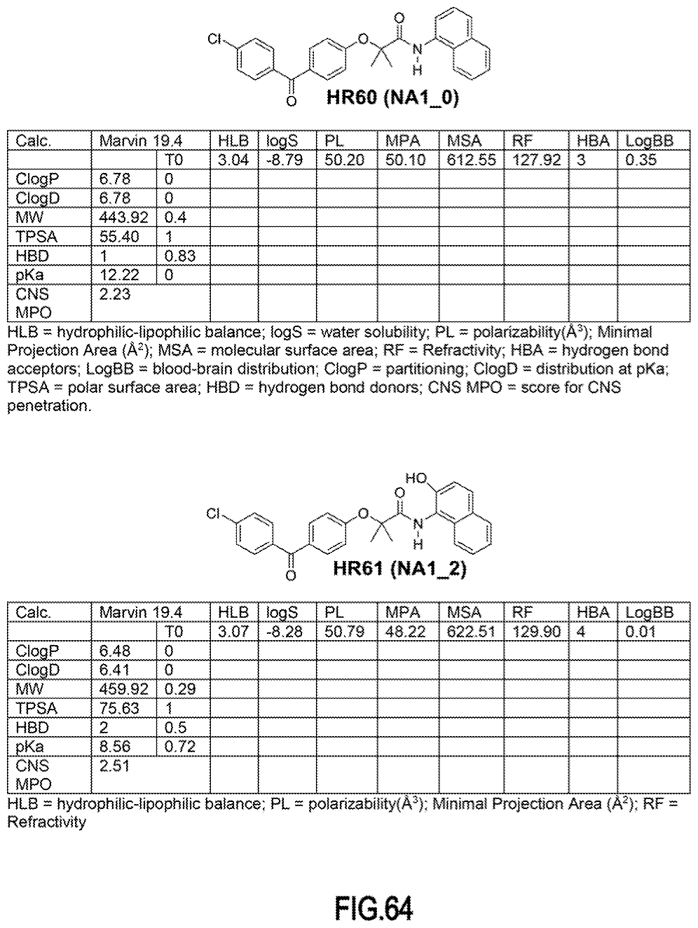

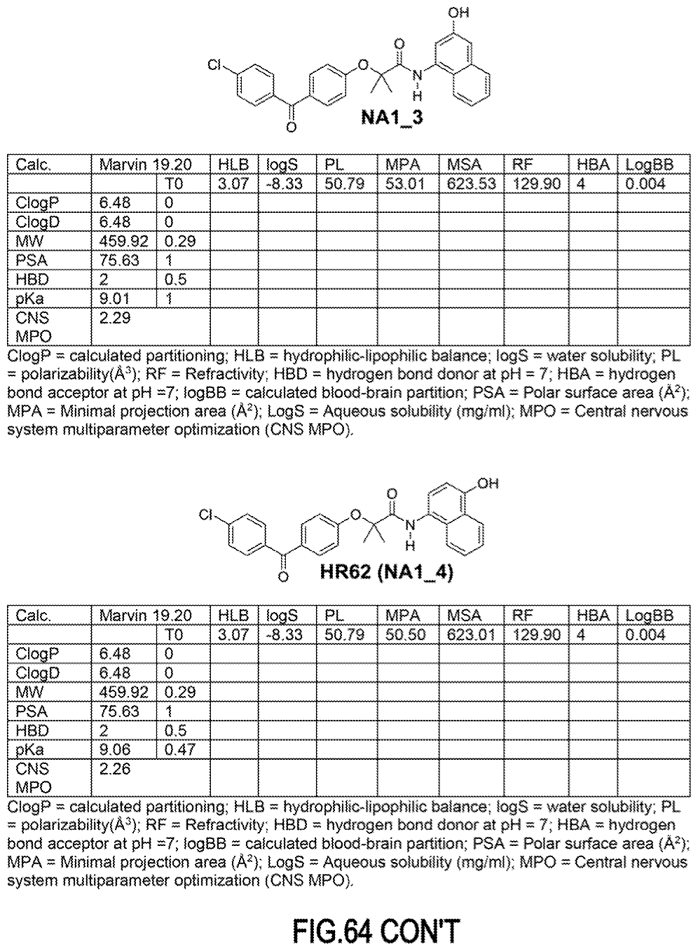

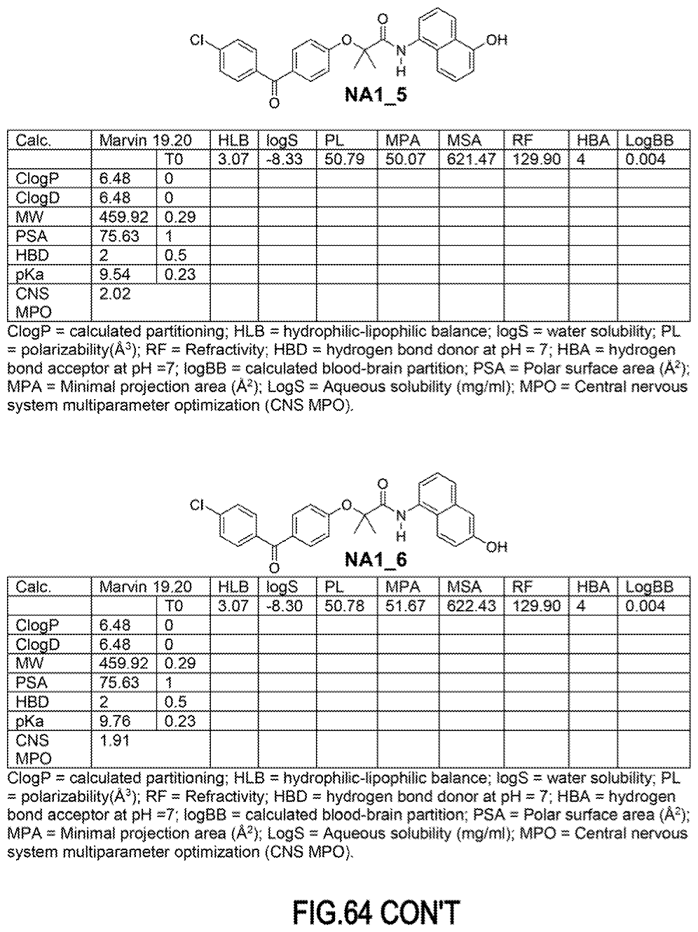

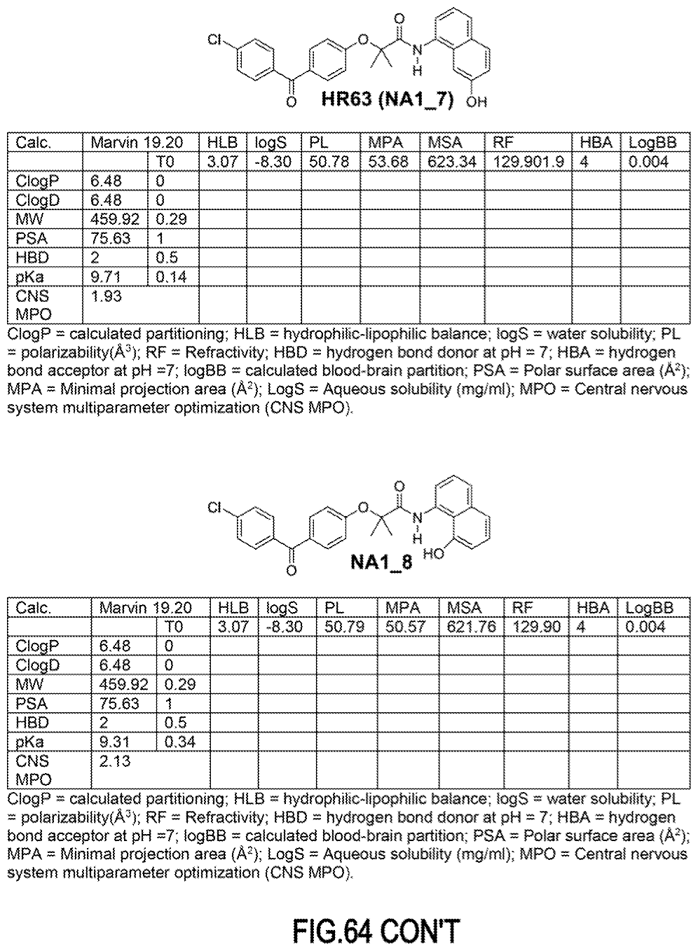

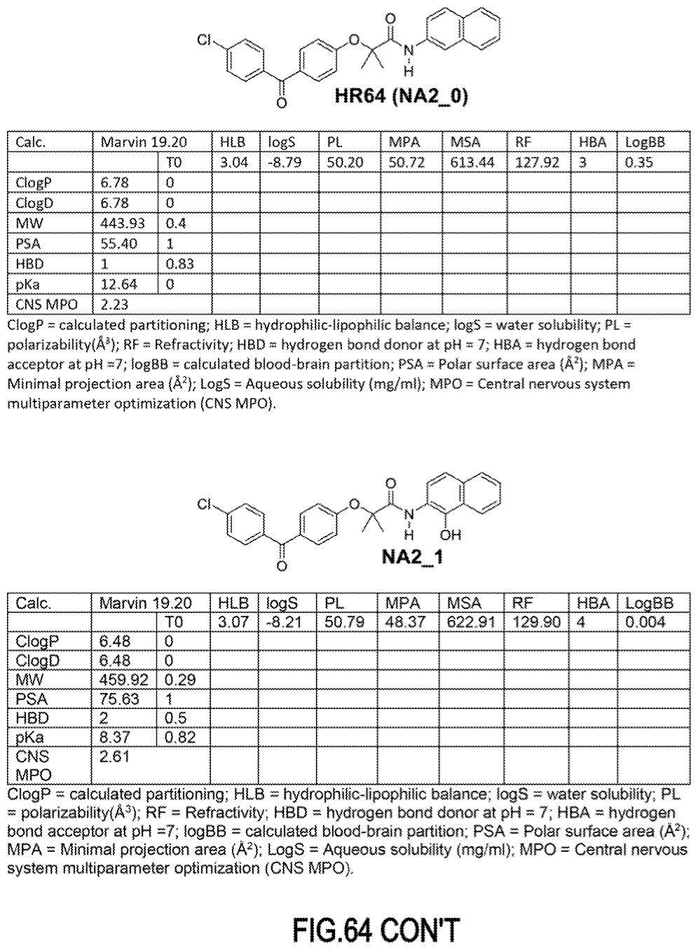

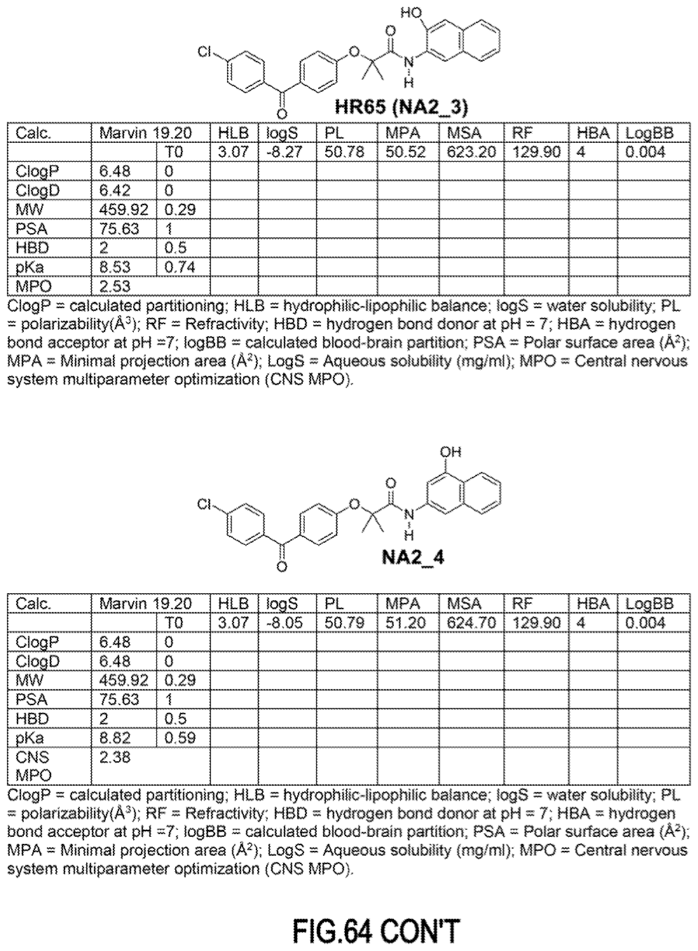

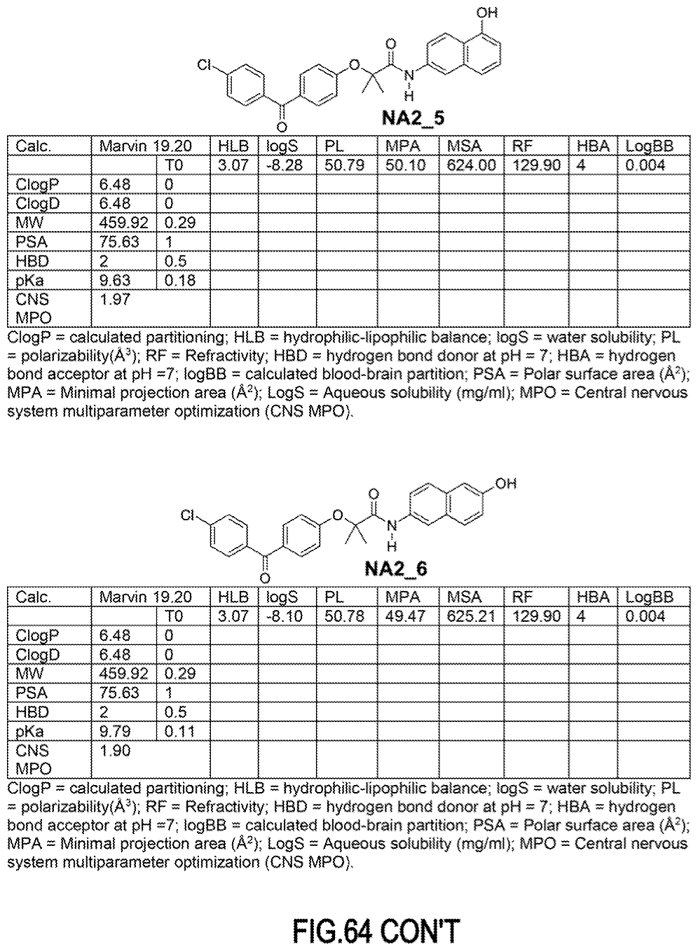

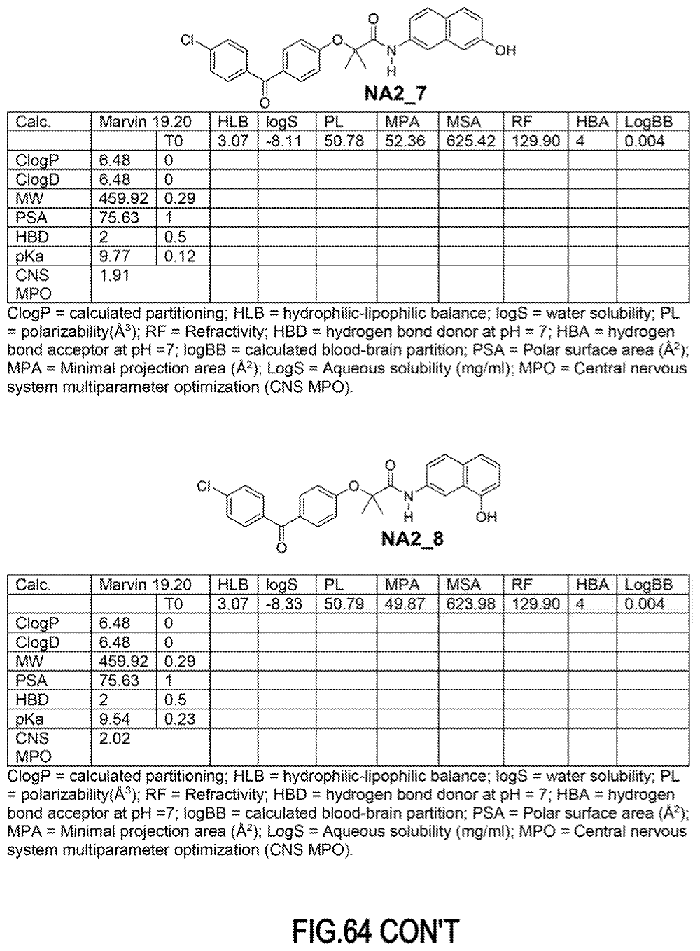

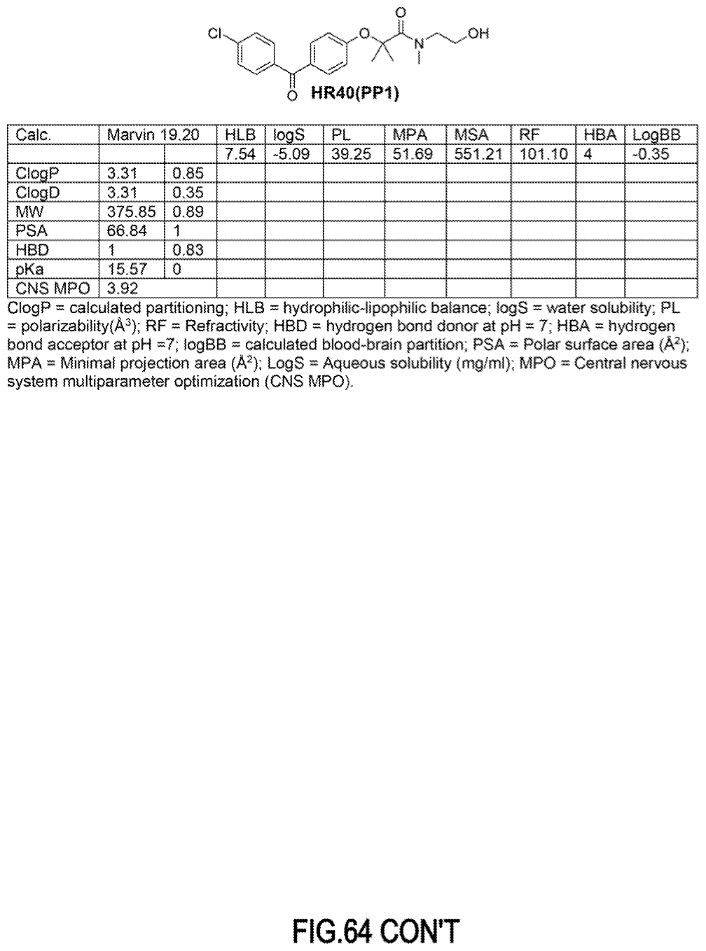

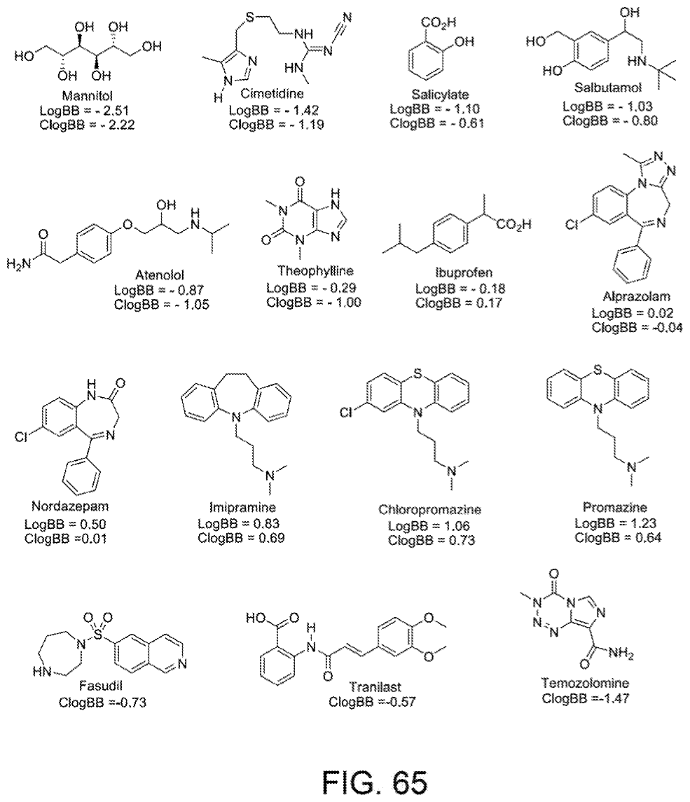

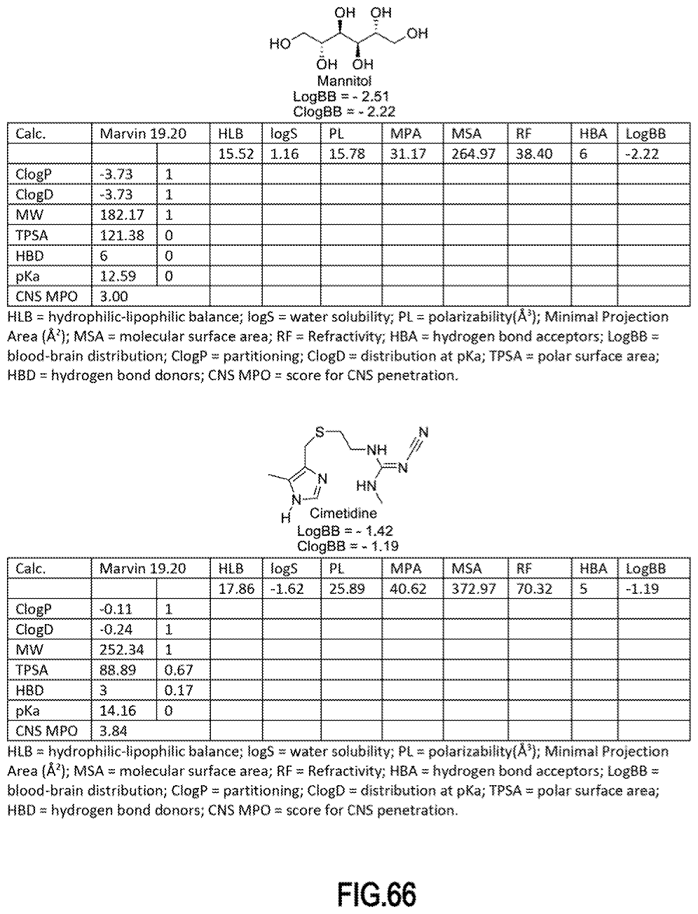

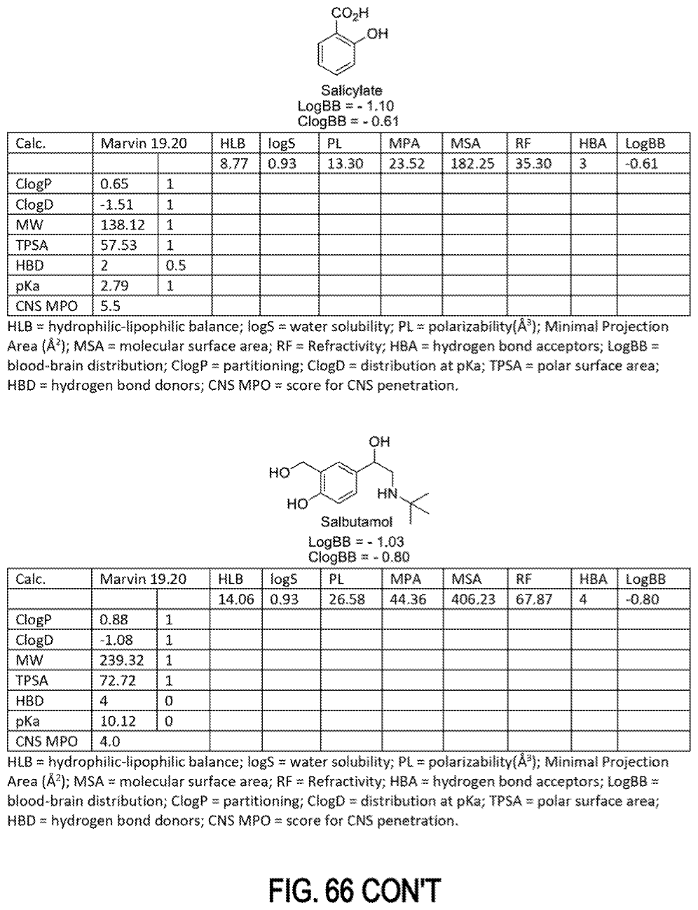

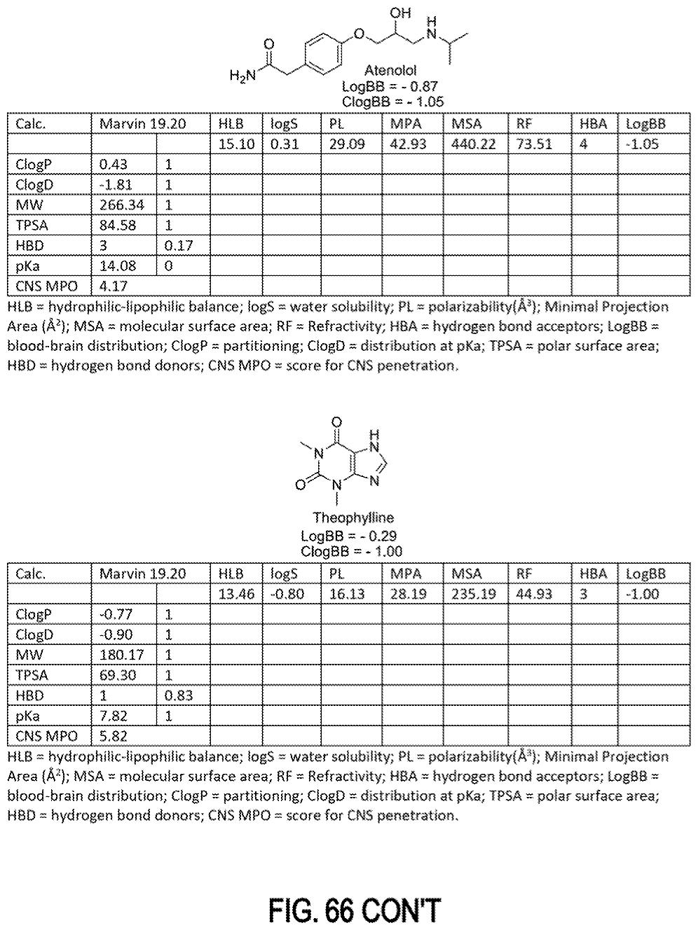

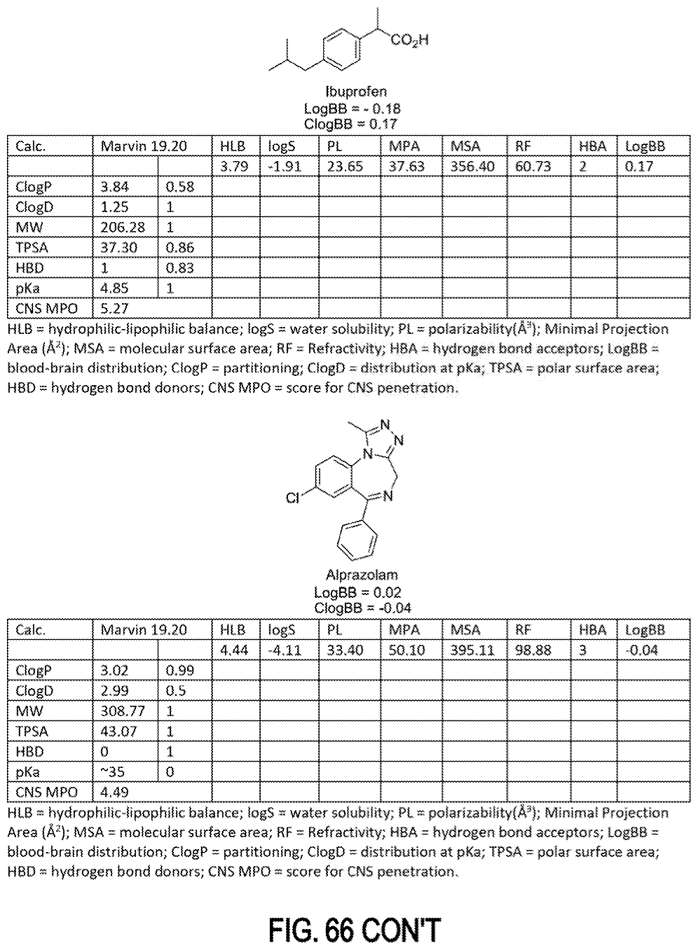

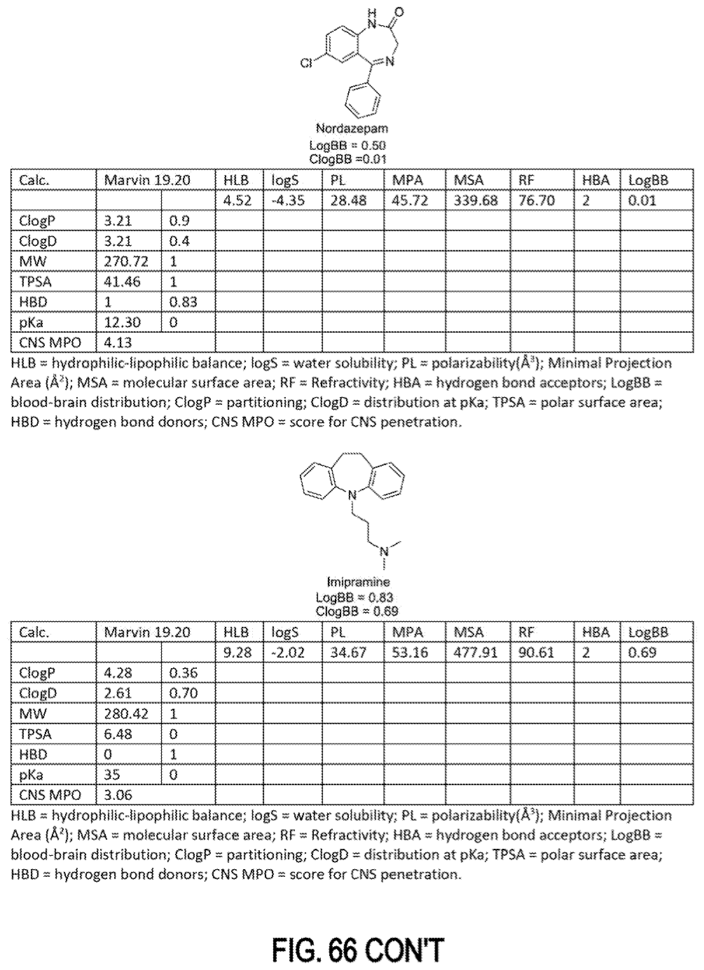

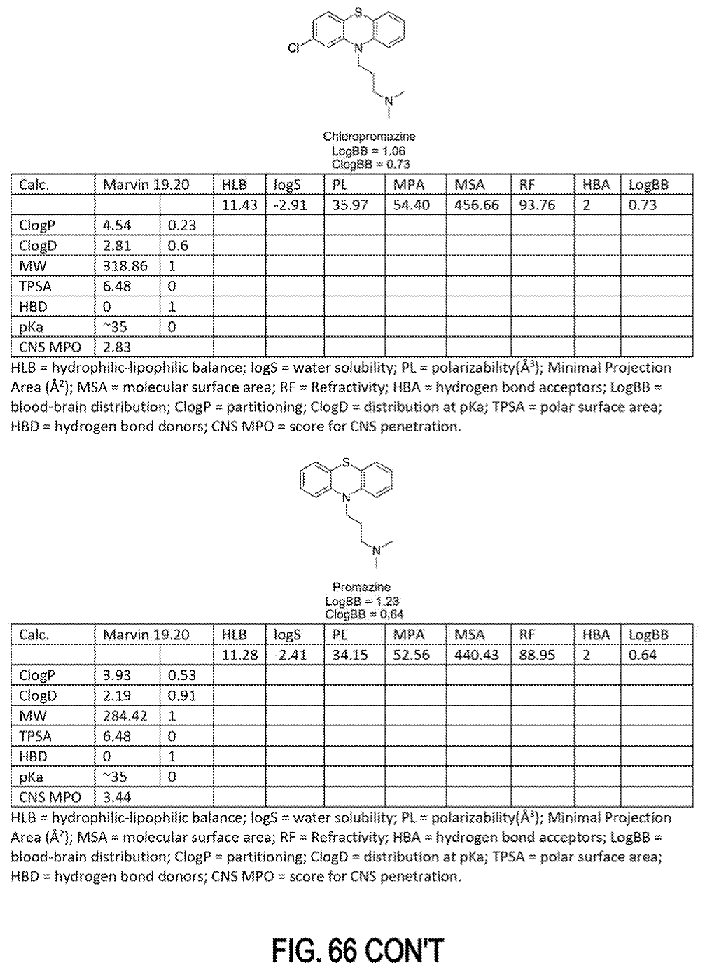

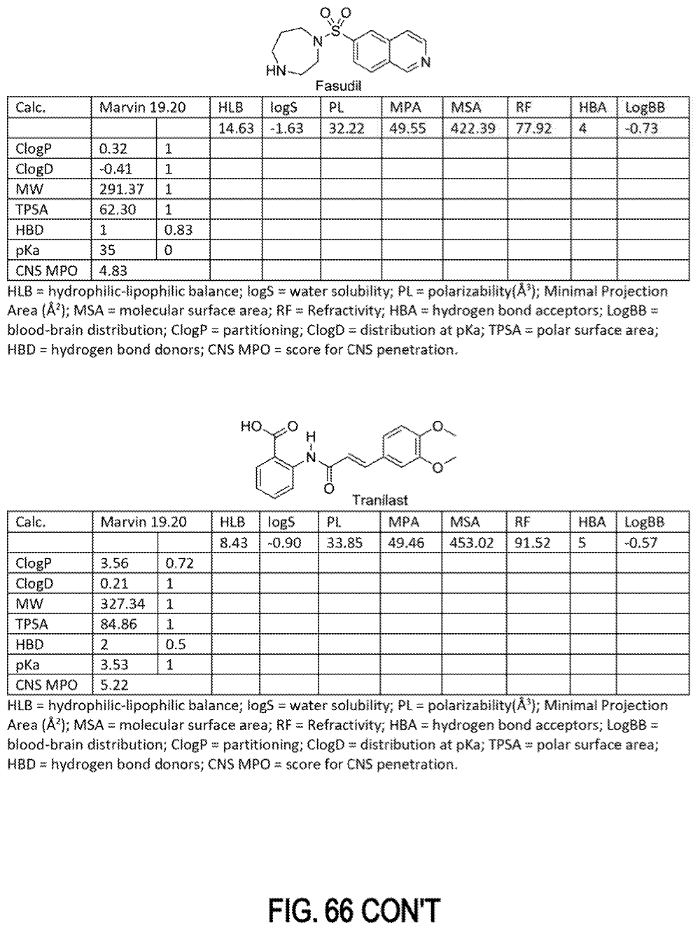

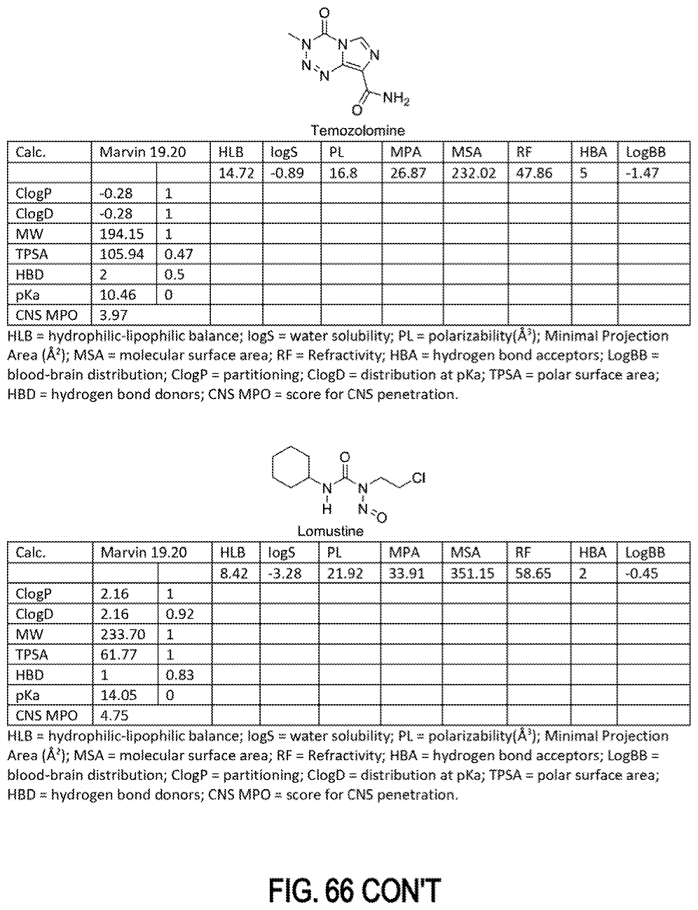

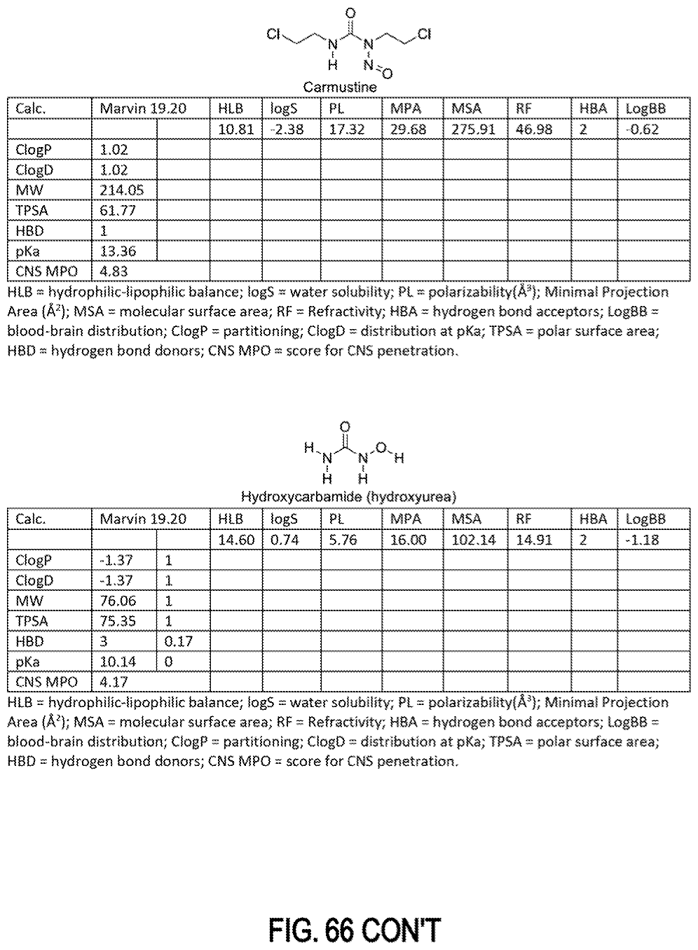

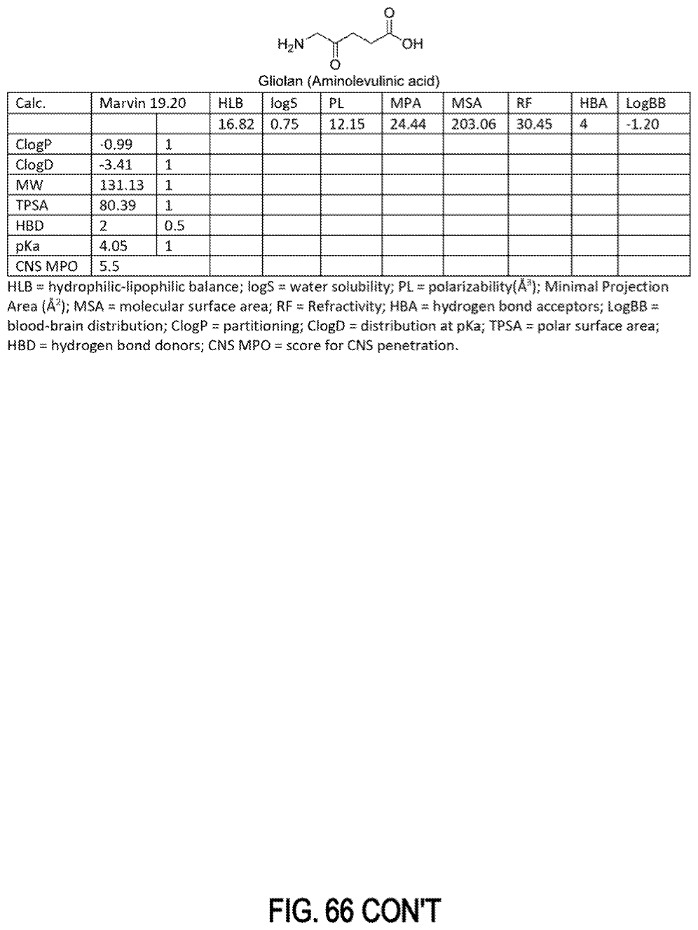

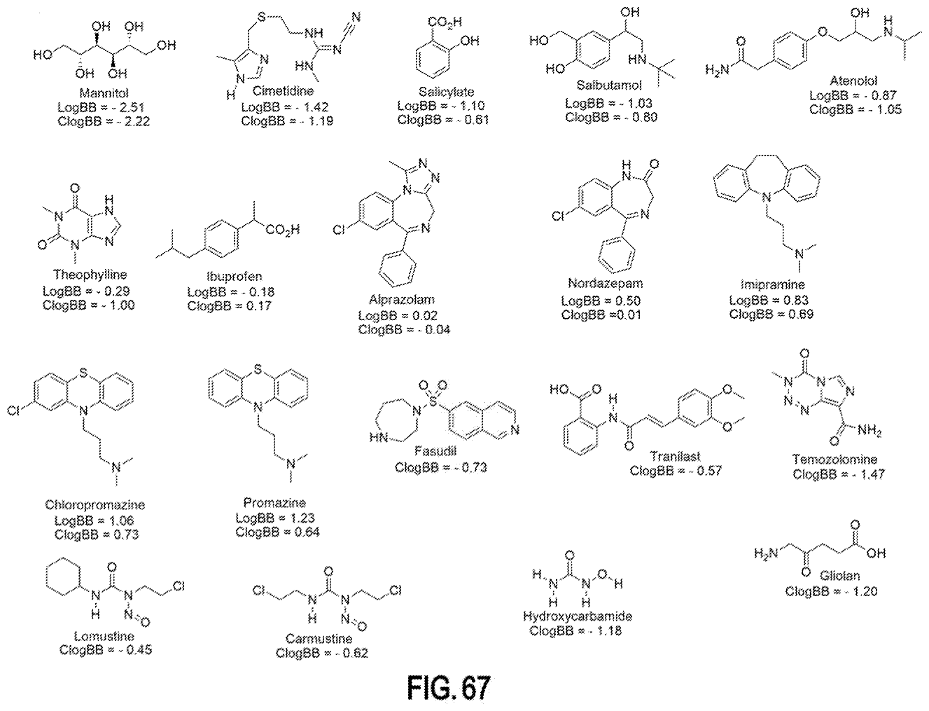

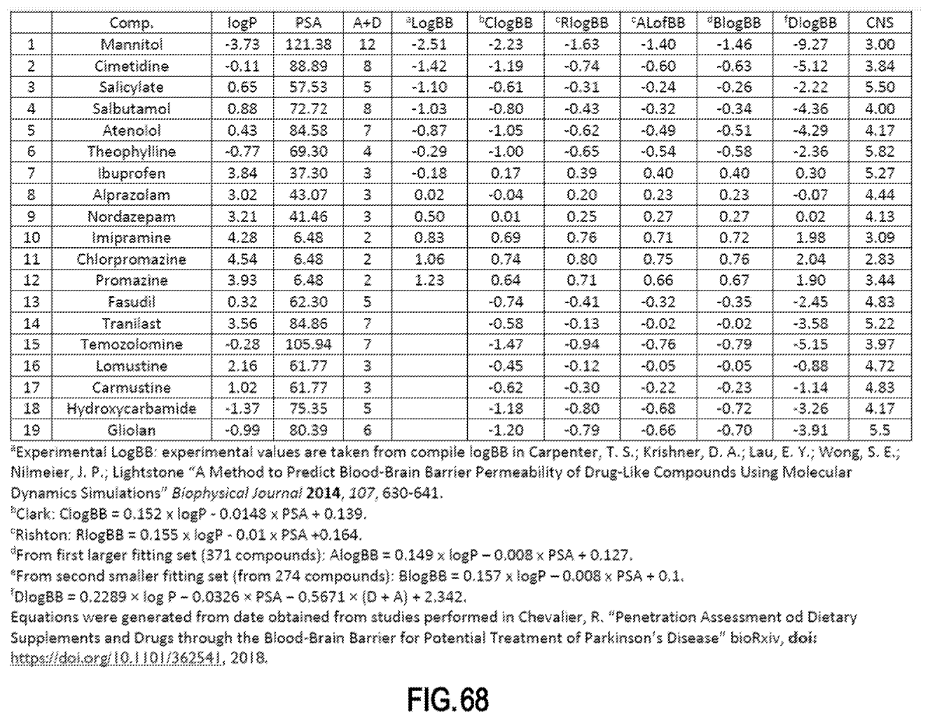

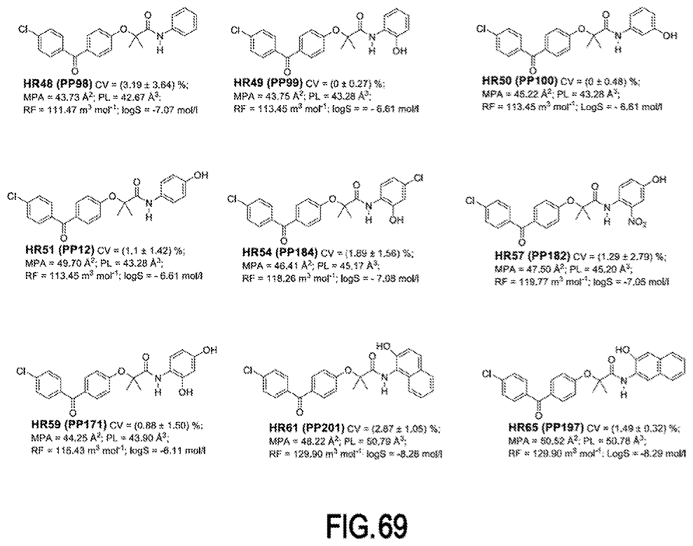

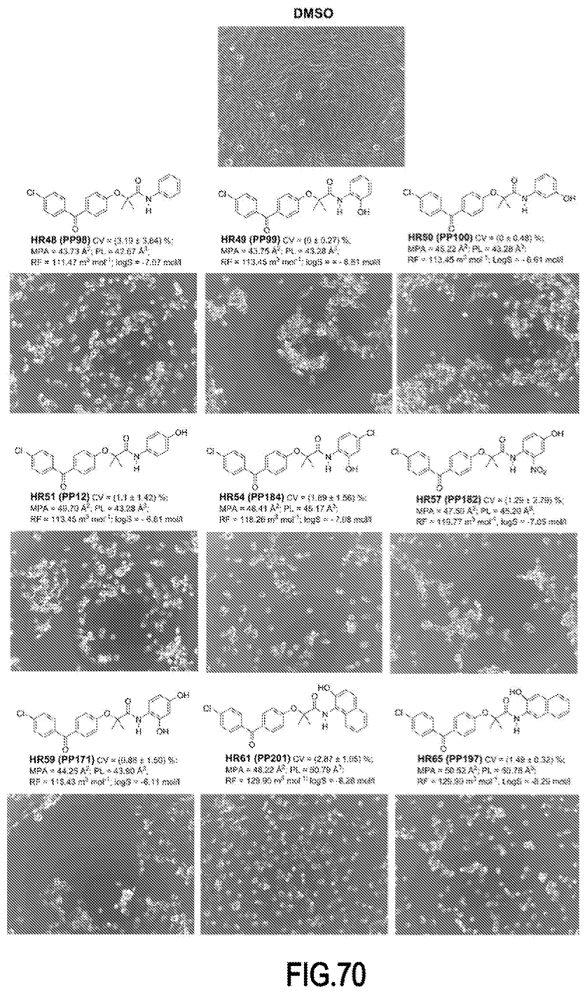

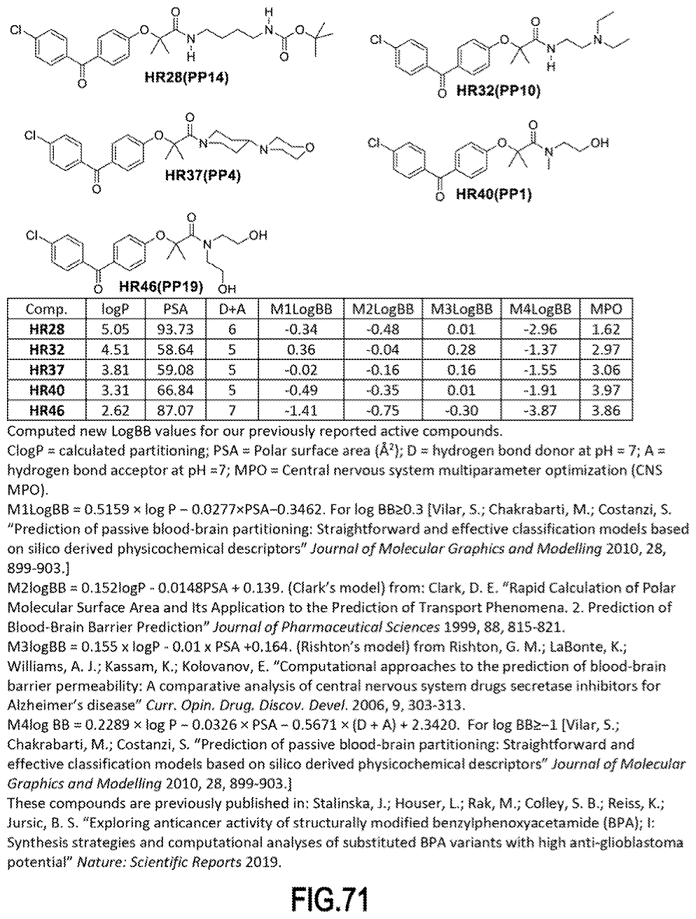

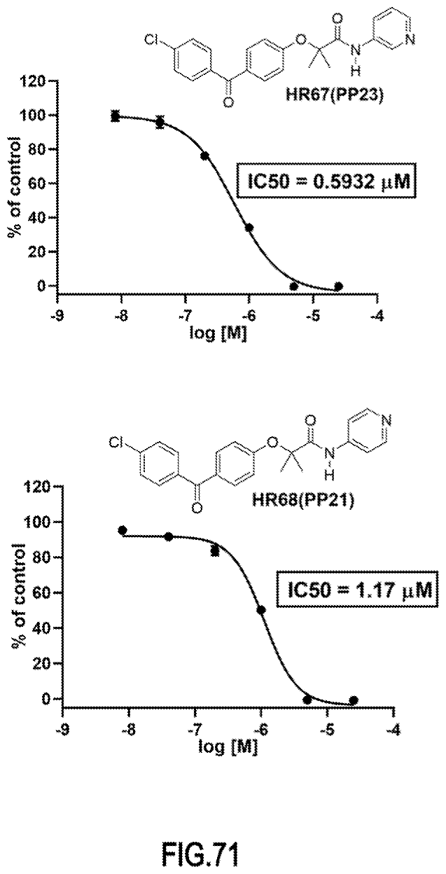

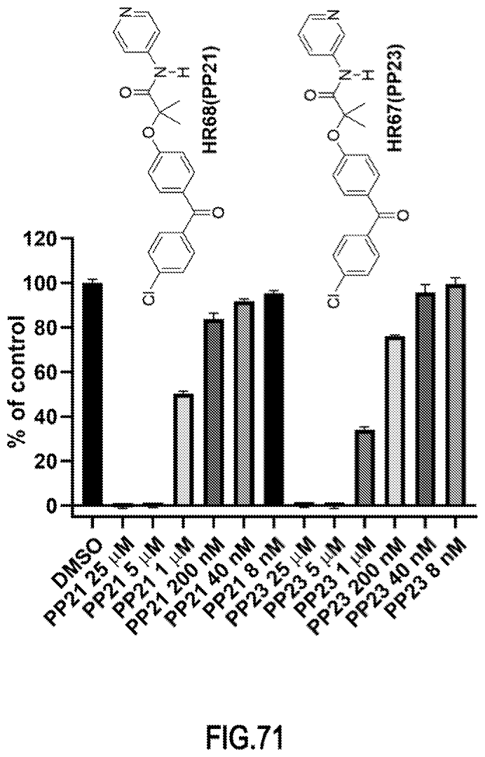

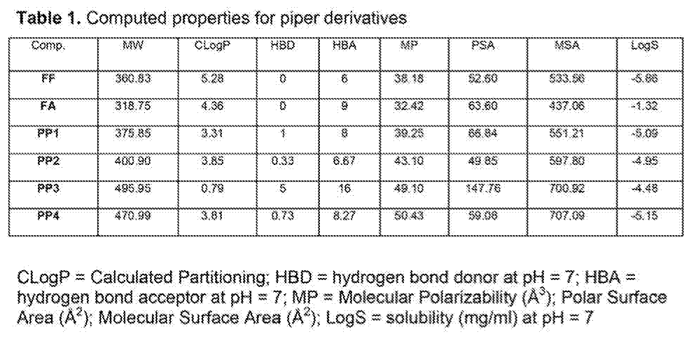

Biological, Chemical and Computational Testing of Therapeutic Compounds: There are many challenges in the development stage of drugs for central nervous system (CNS) [Palmer, A. M; Stephenson, F. A. “CNS drug discovery: challenges and solutions” Drug News Perspect. 2005, 18, 51-57.’ Danon, J. J.; Reekie, T. A.; Kassiou, M. “Challenges and Opportunities in Central Nervous System Drug Discovery” Trends in Chemistry 2019, 1, 612-624.]. Major question is how to find appropriate lead molecular structural skeleton that will result in acceptable drug candidate and ultimately desired drug. In our case there were plethora reported research that some lipid lowering drugs alone or in combination with other anticancer compounds noticeably decrease cancer prevalence [Papanagnou, P.; Stivarou, T.; Papageorgiou, I.; Papadopoulso, G. E.; Pappas, A. “Marketed drugs used for the management of hypercholesterolemia as cancer armament” Onco. Targets Ther. 2017, 10, 4393-4411.; Jang, H. J.; Kim, H. S.; Kim, J. H.; Lee, J. “The Effect od Statin Added to Systematic Anticancer Therapy: A Meta-Analysis of Randomized Controlled Trials” Journal of Clinical Medicine 2018, 7, 325]. On of lipid lowering drug that was used as lead molecule in two of our previous studies is fenofibrate (FF) [Lian, X.; Wang, G.; Zhou, H.; Zheng, Z.; Fu, Y.; Cai, L. “Anticancer Properties of Fenofibrate: Repurposing Use” Journal of Cancer, 2018, 9, 1527-1537.; Majeed, Y.; Upadhayay, R.; Alhousseiny, S.; Taha, T.; Musthak, A. Shaheen, Y.; Jameel, M.; Triggle, C. R.; Ding, H. “Potent and PPARalfa-independent anti-proliferative action of the hypolipidemic drug fenofibrate in VEGF-dependent angiosarcomas in vitro” Nature Scientific Reports 2019, 9, 6316.] from which one we have derived BPA molecular skeleton as an important pharmacophore as anti-glioblastoma drug. Here we evaluate the suitability of phenolic and naphtholic BPA as viable anti-glioblastoma compound. Before any of these compounds were to be prepared intensive computational studies were performed regarding expected physicochemical properties of simple phenoxy BPAs. First group of 3 computed parameters for elimination of drug candidates include log S, Log BB, and MPO-CNS. It was applied on unsubstituted phenolic BPAs (FIG. 41). It is a consensus that these 3 parameters should be consider for any CNS drug design [Hughes, J. P.; Rees, S.; Kalindjian, S. B.; Philpott, K. L. “Principles of early drug discovery” British Journal of Pharmacology 2011, 162, 1239-1249]. In general, it is desirable to have drug candidates with log S higher than −6 [Ganesan, A; Barakat K. “Solubility: A speed-breaker on the drug discovery highway” MOJ Bioequiv. Availab. 2017, 3, 56-58.; Jorgensen, W. L.; Duffy, E. M. “Prediction of drug solubility from structure” Advanced Drug Delivery Reviews 2002, 54, 355-366.], log BB higher than −0.3 [Muehlbbacher, M.; Spitzer, G. M.; Liedl, K. R.; Kornhuber, J. “Qualitative prediction of blood-brain barrier permeability on a large and refined dataset” J. Comput. Aided Mol. Des. 2011, 25, 1095-1106] and CNS-MPO for any organic molecule to be higher than 4 [Wagner, T. T.; Hou, X.; Verhoest, P. R.; Villalobos, A. “Moving beyond Rules: The development of a Central Nervous System Multiparameter Optimization (CNS MPO) Approach to Enable Alignment of Drug Properties” ACS Chem. Neurosci. 2010, 1, 435-449.; Eagner, T. T.; Hou, X.; Verhoest P. R.; Villalobos, A. “Central Nervous System Multiparameter Optimization Desirability: Application in Drug Discovery” ACS Chem. Neurosci. 2016, 7, 767-775.]. However, for molecules that are larger (MW higher than 400 and C log P bigger than 4) are penalized more by their CNS MPO score and desirable values should be around 3 [Mayol-Llinas, J.; Nelson, A.; Famaby, W.; Ayscough, A. “Assessing molecular scaffolds for CNS drug discovery” Drug Discov. Today 2017, 22, 965-969.; Ghose, A. K.; Ott, G. R.; Hudkins, R. L. “Technically Extended MultiParametar Optimization (TEMPO): An Advanced Robust Scoring Scheme To Calculate Central Nervous System Druggability and Monitor Lead Optimization” ACS Chem. Neurosci. 2017, 8, 147-154]. Considering that phenolic BPA is have MW≥400 and log P≥5 our cutoff values for selection, preparation, and biological activity evaluation is log S≥−7, log BB≥−0.7, and MPO-CNS≥2. Computed values for phenyl and phenolic BPA variants HR48-HR51 are presented in FIG. 51. For all these compounds predicted lipophilicity (C log P) is over 5 what is generally accepted to be too high [Di, L.; Kerns, E. H. “Drug-Like Properties: Concepts, Structure Design and Methods: from ADME to Toxicity Optimization” 2^{nd} Edition, Academic Press, 2016.]. Because lipophilicity is important factor of dug balance between water solubility and molecular ability to be absorbed and transported through cell one can expect that these compounds will have lover solubility. Estimated log S or anilide HR48 is −7.07 suggesting that this compound will be practically insoluble in water. However computed log BB by using modified Clark's equation: log BB=0.152 C log P−0.0148PSA+0.139 [Shityakov, S.; Salvador, E.; Pastorin, G.; Foster, C. “Blood-brain barrier transport studies, aggregation, and molecular dynamics simulation of multiwalled carbon nanotube functionalized with fluorescein isothiocyanate” Int. J. Nanomedicine 2015, 10, 1703-1713.] indicates that this compound will be able to penetrate into brain. However computed CNS-MPO value of 2.59 indicates low brain penetration probability. As we mentioned above there is indication in the literature that this scoring substantially underestimates CNS-MPO values for larger molecular systems such as it is case for our compounds. Other parameter that are regularly used to determine possibility for molecular penetration is molecule Minimal Projection Area (MPA) [Cha, H.; Muller, R.; Pos, K. “Switch-Loop Flexibility Affects Transport of Large Drugs by the Promiscuous AcrB Multidrug Efflux Transporter” Antimicrob. Agents Chemother. 2014, 58, 4767-4772]. It was postulated that drugs variants with minimal projection area >70 Å² are less well transported than other substrates. According to our calculations anilide HR48 should transport well through the membrane because its MPA is only 43.73 Å² (FIG. 41). Other very important measurement that has be used to assess drug candidate validity is computes molecular polar surface area (PSA) [Clark, D. E. “What has polar surface area ever done for drug discovery?” Future Med. Chem. 2011, 3, 469-484.]. One of the first study in this regard has pointed out importance of PSA for drug absorption. In this study it was shown that fully absorbed drugs (FA>90%) had a PSA≤60 Å² while drugs that are less than 10% absorbed had a PSA>140 Å² [Palm, K.; Stenberg, P.; Luthman, K.; Artursson, P. “Polar Molecular Surface Properties Predict the Intestinal Absorption of Drug in Humans” Pharm. Res. 1997, 14, 568-571.]. On the other hand, it was demonstrated that upper limit for PSA for molecule to penetrate the brain was 90 Å² [Kelder, J.; Grootenhuis, P. D.; Bayada, D. M.; Delbressine, L. P.; Ploemen, J. P. Polar molecular surface as a dominating determinant for oral absorption and brain penetration of drugs. Pharm Res 16 (1999) 1514-1519.]. For our starting HR48 molecule estimated PSA is only 55.40 Å² indicating that should be both well absorbed and easily penetrated in brain. On top of that this compound show excellent anti-glioblastoma activity (CV=3.19±3.64, FIG. 51).

Certainly, replacing phenyl group of HR48 with phenol group will substantially change physico-chemical properties of new compounds. There are 3 possible isomers HR49, HR50, and HR51 (FIG. 51). By replacing hydrogen atom with OH group all parameters are changed in direction of water solubility, absorption, as well as brain penetration. Lipophilicity (C log P) is reduced and closer to the desirable value of below 4, solubility is higher and both PSA and MPA are in desirable range while CN-MPO numbers are not change substantially (FIG. 51). All computed parameters suggest that these 3 phenolic BPAs should be better anti-glioblastoma drug candidates than HR48. If we explore electrostatic potential of phenolic BPAs we can se that there is not substantial change in molecular shape but there is noticeable difference in their electrostatic density. While electro-deficient area is located on BPA skeleton (Left side of the pictures) high electron dense area is on the phenolic part of the molecule. Electrostatic potential maps reveal molecular spatial area that are of vital importance for drug-targeted biomolecule interactions [Rathi, P. C.; Ludlow, F.; Verdonk, M. L. “Practical High-Quality Electrostatic Potential Surfaces for Drug Discovery Using a Graph-Convolutional Deep Neural Network” J. Med. Chem. 2019, 62, 000.; Kumar, A.; Zhang, K. Y. “Advances in the Development of Shape Similarity Methods and their Application in Drug Discovery” Front. Chem. 2018, 6, 315]. Good example electrostatic complementarity between protein and ligand was described recently [Bauer, M. R.; Mackey, M. D. Electrostatic Complementarity as a Fast and Effective Tool to Optimize Binding and Selectivity of Protein-Ligand Complexes. J. Med. Chem. 2019, 62, 3036-3050]. Electrostatic potential maps are intrinsically interlaced with frontier molecular energy and population [Braga, E. J.; Corpe, B. T.; Marinho, M. M.; Marinho, E. S. Molecular electrostatic potential surface, HMO-LUMO, and computational analysis of synthetic drug Rilpivirine” International Journal of Scientific & Engineering Research 1 2016, 7, 315-319. Frontier orbitals (HOMO-LUMO) are important parameters in assessing drug likeliness in serious of similar compounds [Lever, G.; Cole, D. J. Hinem N. D.; Haynes, P. D.; Payne, M. C. “Electrostatic considerations affecting the computed HOMO-LUMO gap in protein molecules” J Phys.: Condens. Matter 2013, 25, 152101; Mehmood, A.; Jones, S. I.; Tao, P.; Janesko, B. G. “An Orbital-Overlap Complement to Ligand and Binding Site Electrostatic Potential Maps” Journal of Chemical Information and Modeling 2018, 58, 1836-1846.]. Electrostatic pontantial map indicates higher electron density on the anilide (right) part of HR48 and lover electron density in BPA (left) part of HR48 (FIG. 41). That is perfectly demonstrated that LUMO (Lowest Unoccupied Molecular Orbitals) are located on the BPA part of the molecules while HOMO (Highest Unoccupied Molecular Orbital) is in anilide part of the molecule. There is postulation that majority of active region of the biomolecular targets are electron deficient [Pang, X.; Zhou, L.; Zhang, M.; Zhang, L.; Xu, L.; Xie, F.; Yu, L.; Zhang, X. “Two Rules of the Protein-Ligand Interation” The Open Conference Proceedings Journal, 2012, 3, 70-80.; Srivastava, A.; Singh, H.; Mishra, R.; Dev, K.; Tandon, P.; Maurya, Rakesh “Structural insights, protein-ligand interations and spectroscopic characterization of isoformononetin” J. Mol. Struct. 2017, 1133, 479-491.; Balajee, R.; Srinivasadesikan, V.; Sakthivadivel, M.; Gunasekaran, P.; “In Silico Screening, Alannine Mutation, and DFT approaches for Identification of NS2B/NS3 Protease Inhibitors” Biochem. Res. Int. 2016, 2016, 7264080]. If we accept this approach for our studies it is therefore important oncrease electron density on anilide part of HR48 by introducing hydroxy group in otho, metha, and para position and in this way generate three new phenolic BPAs (HR49, HR50, and HR51; FIG. 41). Although geometry of the molecular skeleton does not change substantially electrostatic potential maps indicates noticeable increase of “negative” electrostatic surface on the phenolic part of the molecules indicating substantial electron shift. That is well reflected in calculated molecular polarizability that is increased from 42.67 for HR48 to 43.28 for phenolic BPAs (FIG. 41). Molecular polarizability crucial for ligands ability to adapt its electrostatic potential to be complementary to the ligand (protein) [Bryce, A. R. “Physics-based scoring of protein-ligand interactions: explicit polarizability, quantum mechanisms and free energies” Future Med. Chem. 2011, 3, 683-698]. Therefore, without wishing to be bound by theory, phenolic BPAs will have stronger interactions with targeted protein(s). Additional measurement is dipole moment of the molecule that follows the same trend with higher dipole moment for HR50.

As mentioned before frontier molecular orbitals are routinely used in molecular modeling for drug discovery [Aminpour, M.; Nontemagno, C.; Tuszynski, J. “An Overview of Nolecular Modeling for Drug Discovery with Specific Illustrative Examples of Applications” Molecules 2019, 24, 1693]. The atomic orbital contribution to LUMO is entirely from the BPA molecular part and therefore it is not surprising that their estimated energies are almost identical (from −6.66 to −6.72 eV; FIG. 4). On the other hand, predicted HOMO energies differ substantially. Higher HOMO energy should also indicate stronger interactions of our phenolic BPA with targeted biomolecule. So according to all of our computed descriptors phenolic BPA should have better anti-glioblastoma activity than anilide BPA. This was confirmed with measuring their cell viability. All phenolic BPAs (HR49, HR50, and HR51) are more potent than HR48.

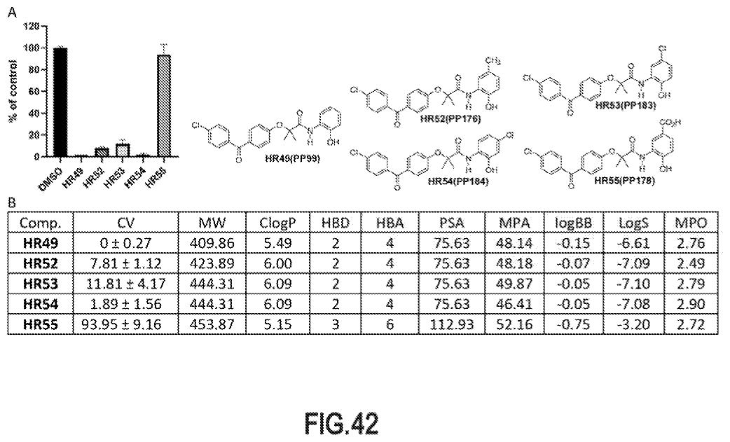

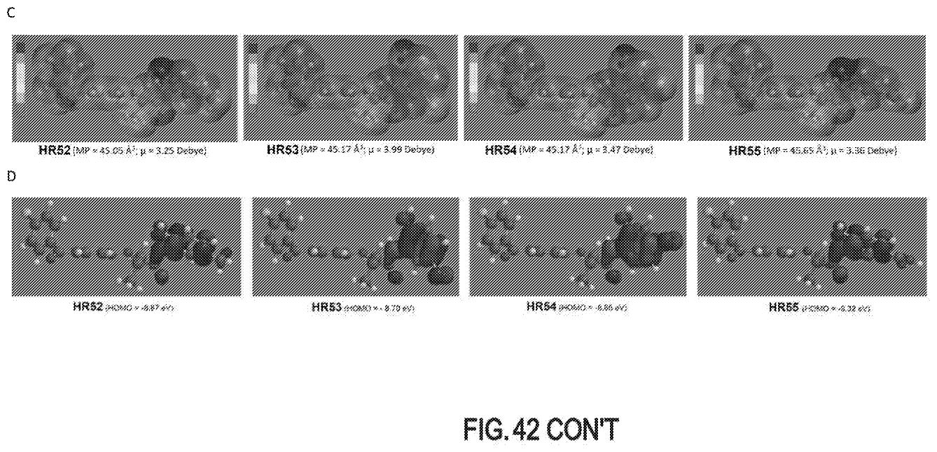

One can consider that HR49 is the most potent compounds of three phenolic BPAs. We have demonstrated that with changing electrostatic potential surface one can alter ant-glioblastoma activity of these compounds. We place on the phenol ring moderate electron donating (methyl), slightly electron withdrawing (chloro) and strong mesomeric electron withdrawing group (carboxylic). Computational studies all of 30 isomers were performed and four candidates were selected to be prepared and their anti-glioblastoma activity was evaluated (FIG. 42). Off cause for all methyl and chloro derivatives generated from HR49 have higher molecular weight and lipophilicity, what is directly associated with lower solubility of these compounds. There is only a slight increase in brain blood penetration prediction (log BB). On the other hand, presence of carboxylic acid substantially increases the solubility, but computed blood brain penetration is noticeably lower that suggests that these compounds are cannot be potent anti-glioblastoma agents. Considering only these four compounds HR54 has best estimated physicochemical properties: estimated CNM MPO value is the highest (2.90) and minimal projected area is only 46.41 Å². Considering that molecular polarizability is substantially higher then in the case of HR49 one can argue that when this compound penetrated in CNS it might be more potent. However estimated HOMO energies for both HR49 and HR54 are identical (˜8.66 eV). It seems that beneficial effect of chlorine atom on phenolic BPAs such as predicted better blood penetration properties are diminished with predicted lower water solubility, too high molecular polarizability, too high electrostatic potential surface separation and consequently too high dipole moment in comparison with HR49 to make it better anti-glioblastoma drug candidate. This was in full agreement with observed cell viability values that are slightly worse than in the case HR49.

Almost all computed indicators for carboxylic acid derivatives of phenolic BPAs indicate that these compounds are not good anti-glioblastoma drug candidates. To demonstrate this finding only one typical example of these group of compounds were presented. Compound HR55 has only one desirable physicochemical property that are better than for good glioblastoma drug candidate phenolic BPA HR49. Its water solubility (log S for HR55 is −3.20 in comparison with −6.61 for HR49) may be better. To demonstrate large deviation for desirable physicochemical properties when screen for molecular targets we will focus on basic principles of computational studies [Pajouhesh, H.; Lenz, G. R. “Medicinal Chemical Properties of Successful Central Nervous System Drugs” NeuroRx 2005, 2, 541-553]. Introducing carboxylic acid group in HR55 compounds is an acid with computed pKa of 4.23. Therefore, at philological pH this compound is deprotonated, and it is more important to use log D (partition dependence regarding pH) that is 2.13 [Kokate, A.; Li, Z.; Jasti, B. “Effect of drug Lipophilicity and Ionization on Permeability Across the Buccal Mucosa: A Technical Note” AAPS Pharm Sci Tech 2008, 9, 501-504; Lindsley C. W. “Lipophilicity. In: Stolerman I., Price L.; Price L. H (eds) Encyclopedia of Psychopharmacology” 2014 Springer, Berlin, Heidelberg.]. In comparison estimated log D for HR49 is 5.1. In addition, electrostaic potential map indication noticeable change in the phenolic part of the molecule. Considering that this is electron rich expected HVOMO energy should be substantially altered (−8.65 vs −9.32 respectively) while LUMO energy should be basically unchanged (−0.66 vs −0.69 eV respectively). That was perfectly demonstrated in FIG. 42. Visually compared HOMO molecular orbitals for HR52-HR55 are almost identical and located exclusively on the BPA part of the molecule (Panel D FIG. 42) while HOMO energy located on the phenolic part of the molecules is slightly altered (Panel E FIG. 42). Considering that shape and higher energy of the HOMO orbital is important for ligand binding it is estimated that HR55 should be noticeably less potent than HR49. One other parameter that is regularly used for study drug formulation is HyWdrophilic-Lipophilic Balance (HLB) [Breuer, E.; Chorghade, M. S.; Fischer, J.; Golomb, G. “Hydrophile-Lipophile Balance System (HLB System) From: Glossary of Terms Related to Pharmaceutics” Pure Appl. Chem. 2009, 81, 971.; Soundharya, R.; Aruna, V.; Aniruthavalli, G. V.; Gasathri. R. “Inclusion of Hydrophilic-Lipophilic Balance (HLC) in the treatment of Psoriasis—A new approach” International Journal of Advances in Pharmaceutics 2019, 8, e5150,]. Estimated HLB for HR55 is 6.29 in comparison to 4.53 for HR49, Higher HBL indicates stronger interactions with water and lower drug penetration resulting in diminishing biological activity. Compounds for which computed electronic properties do not deviate substantially from computed electronic properties of HR49 is HR54 except predicted lower water solubility. This was perfectly demonstrated with experiment cell viability on LN229, Drug candidate HR54 is just slightly less active than HR49 while HR55 is practically not active at all at 25 μM concentration (Panel B).

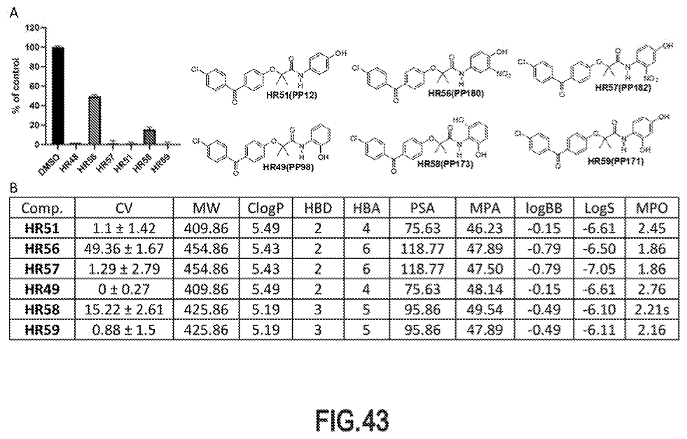

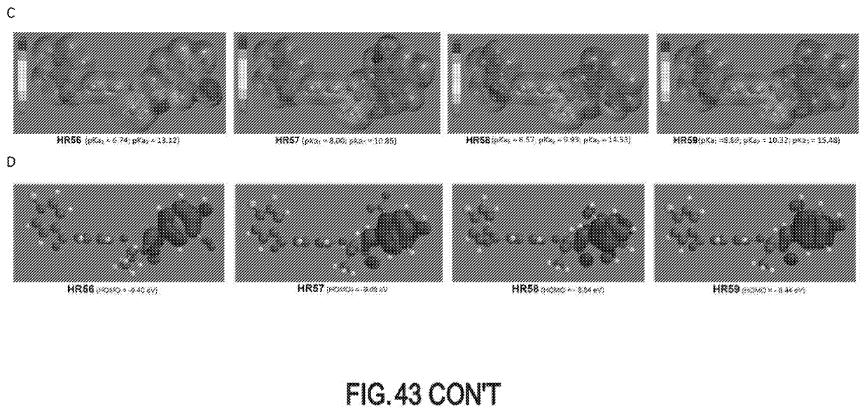

One can argue that one contribution to the phenolic BPA activity (FIG. 41, Panel B) comes from phenol hydrogen bonding (donor) capability expressed through their pKa values because their (HR49, HR50, and HR51) experimental cell viability (CV) perfectly revers correlate to their pKa vales. Two groups can substantially alter electronic distribution and acidity of phenols is two diametrically opposite groups: additional hydroxy group as strong electron donating group and nitro group as strong electron withdrawing group. Extensive computational studies of all these structural variations were performed only two with nitro group (HR56 and HR57 as structural variants of HR51) and two hydroxy group (HR58 and 59 as structural variant of HR49) (FIG. 43 Panel A). Electrostatic potential maps are substantially altered with these two groups added. Nitro groups substantially decrease electron density on the phenolic moiety and therefore makes HOMO energy very low indicating that electrostatic interactions with targeted biomolecule should be diminished. This is much more profaned in HR56 than in HR57 due to presence of hydrogen bonding between neighboring nitro and hydroxy groups. Therefore, predicted pKa1 are 6.74 and 8.00 respectively. Because of relatively low pKa1 the log D must be taken in the account. The computed log D for HR56 is 4.62 and for HR57 is 5.27. If we couple this with HOMO energies (FIG. 43 Panel D) comparison with phenolic HR51 that is 5.48 indicated diminishing penetration ability. In addition, estimated polar surface area are relatively high (118.77 Å²) and its is un the upper border of acceptability. Combining that with their HOMO energy (−9.40 eV, −9.09 eV, and −8.81 eV for HR56, HR57, and HR51 respectively) predict that their order of the potency will be HR56<HR57<HR51. That order of activity was also confirm experimentally making HR57 slightly less potent and HR56 substantially less potent than HR51. However estimated solubility for HR57 (log S 7.05 mg/ml) is too low to be consider as viable anti-glioblastoma drug candidate.

Introducing hydroxy group will substantially increase electron density on the phenolic moiety as well as solubility in water due to one more hydrogen donor group. One and two hydroxy derivatives of phenolic BPA (di and triphenols) was subject of our computational studies. From 11 compounds we have selected HR58 and HR59 candidates with best computed physicochemical descriptors for preparation and their anti-glioblastoma activity evaluation. These two compounds, HR58 and HR59, can be considered as monohydroxy derivatives of HR49 (Panel A). Both computed lipophilicity and water solubility are at an acceptable level. The predicted lipophilicity is slightly higher and predicted water solubility is slightly lower than what would be optimal values. There is not substantial difference in pKa1 values and ˜8.6 these molecules are unionized form at physiological pH. Predicted PSA are in an acceptable range. Major difference between these two compounds is in minimal projected area and HOMO energies. It is predicted that HR59 will penetrated slight better (MPA is 47.89 vs 49.54 Å² for HR58, Panel A) and it is predicted to bind slightly stringer (HOMO is −8.54 vs −8.44 eV for HR58). Therefore, without wishing to be bound by theory, HR59 is more potent than any of the four studied drugs candied in this series of phenolic BPA. In fact, it is experimentally confirmed that this is the most potent compound and it should be considered viable for further studies as anti-glioblastoma drug.

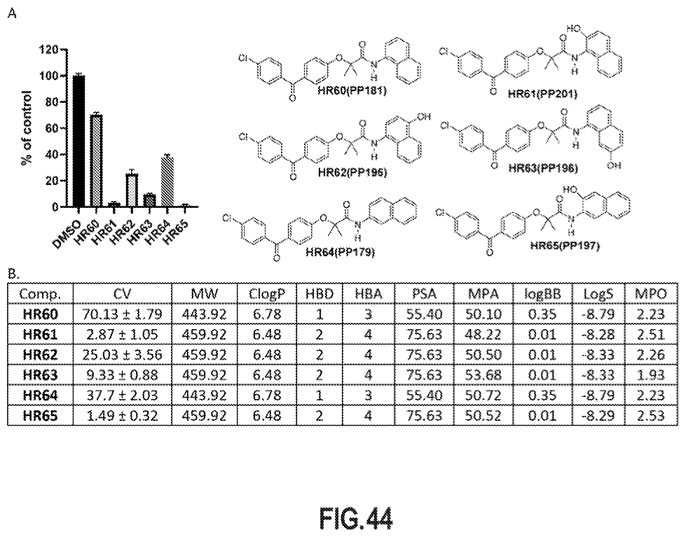

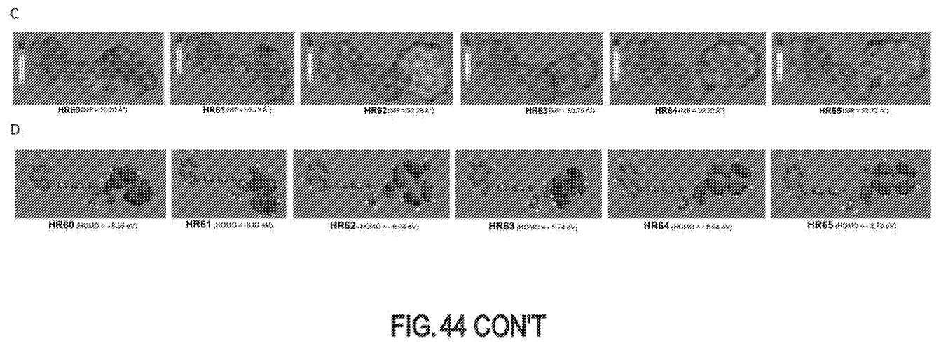

Both computational studies and experimental data confirm that phenolic BPAs are an excellent anti-glioblastoma drug candidate. Beside lipophilicity, water solubility, blood-brain distribution, molecular size, frontier orbital energy (particularly HOMO), and electrostatic potential maps seems to be additional selecting physicochemical parameters to determine molecular suitability as anti-glioblastoma drug candidate. Computational studies of 1- and 2-naphylyl derivatives of BPA and 14 corresponding naphthols of BPA are performed. From this study we have selected six compounds for synthesis and further studies. Results are presented in FIG. 44. Considering that additional aromatic ring was fused to phenolic moiety it is not surprise that for all of these compounds' high lipophilicity and low water solubility was predicted. In fact, estimated log P is between 6.4-6.8 and water solubility (log S is well below −8 mg/ml Panel A). Only better parameter than for phenolic BPAs is log BB but this is deceiving because due to water solubility blood concentration (bioavailability) should be exceptionally low [Wang, J.; Krudy, G.; Hou, T.; Zhang, W.; Holland, G.; Xu., X. “Development of Reliable Aqueous Solubility Models and Their Application in Druglike Analysis” J. Chem. Inf. Model. 2007, 47, 1395-1404.] and consequently their accumulation in brain negligible. Predicted polar surface area are around 75 Å² and are in recommended range. Same is true for minimal projected area of ˜50 Å² that is in the range of molecule that are capable to penetration the membrane (It was suggested that compounds with higher than 50 Å² do have lover probably to be absorbed and that 70 Å² is to be considered as a cut off for any further drug evaluation. Cha, H.-J.; Muller, R. T.; Pos, K. M. “Switch-Loop Flexibility Affects Transport of Large Drugs by the Promiscuous AcrB Multidrug Efflux Transporter” Antimicrob. Agents Chemother. 2014, 58, 4767-4772.]. In naphthalene series frontier HOMO orbital energy is not in direct correlation with observed LN 229 cell viability data (Panel B). Without wishing to be bound by theory, simple naphthyl derivatives of BPA such as HR60 (with HOMO energy of −8.55 eV) and HR64 (with HOMO energy of −8.64 eV) should be more active than naphthol BPA such as HR61 (with HOMO energy of −8.67 eV) and HR65 (with HOMO energy of −8.73 eV). Low solubility of these compounds is so low that slight preference is give one that have marginally better solubility (HR61 and HR65 over HR60 and HR64; Table 7, Panel B) regardless of the HOMO energy preference (Panel D). Molecular polarizability clearly favors naphthol BPAs (Panel C). Experimental data select HR61 and HR65 that have phenol (hydroxy) group in ortho position as more potent drug candidates. The cell viability data om LN229 are acceptable but their low water solubility may limit their use anti-glioblastoma drug candidates.

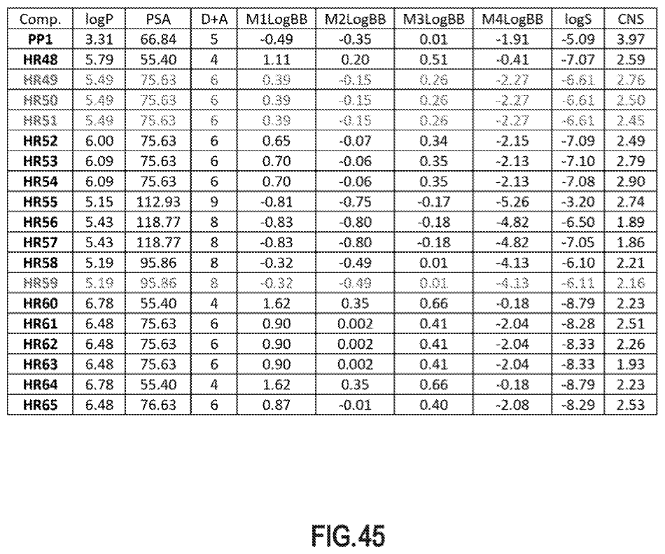

As mentioned above it is very difficult for drug to penetrated Central Nervous System. It is absolute necessity to develop reliable models that can accurately predict drugs blood-brain-barrier penetration ability [Gribkoff, V. K.; Kaczmarek, L. K. “The Need for New Approaches in CNS Srug Discovery: Why Drugs Have Failed, and What Can be Done to Improve Outcomes” Neuropharmacology 2017, 120, 11-19.; Banks, W. A.; Greig, N. H. “Small molecules as central nervous system therapeutics: old challenges, new directions, and a philosophic divide” Future Med. Chem. 2019, 11, 489-493.; Miao, R.; Xia, L.-Y.; Chen, H. H.; Huang, H.-H.; Liang, Y. “Improved Classification of Blood-Brain-Barrier Drugs Using Deep Learning” Scientific Reports 2019, 9, 8802.; Plisson, F.; Piggott, A. M. “Predicting Blood-Brain Barrier Permeability of Marine-Derived Kinase Inhibitors Using Ensemble Classifiers Reveals Potential Hits for Neurodegenerative Disorders” Mar. Drugs 2019, 17, 81.]. We used several models to calculate log BB values and compare them to experimental values to select best one that is suitable to our studies. We have applied these models to evaluate brain penetration ability of our compounds presented in this paper as well as for our earlier BPA compound PP1 that penetrates the brain (5 μM) with its estimated log BB to be zero [Stalinska, J.; Zimolag, E.; Pianovich, N. A.; Zapata, A.; Lassak, A.; Rak, M.; Dean, M.; Ucar-Bilyeu, D.; Culicchia, F.; Marrero, L.; Del Valle, L.; Sarkaria, J.; Peruzzi, F.; Jursic, B. S.; Reiss, K. “Chemically Modified Variants of Fenofibrate with Antiglioblastoma Potential” Trans. Oncol. 2019, 12, 895-907.]. Calculated values are presented in FIG. 45. There are several different models depending what should be expected log BB: M1 Log BB is for molecules that have log BB above 0.3 (good blood penetration). M2 Log BB is clark's model use in Figures above. The best model that we calibrated for known compounds with Log BB values is M3 Log BB—Rishton's model [Rishton, G. M.; LaBonte, K.; Williams, A. J. Kassam, K.; Kolovanov, E. “Computational approach to the prediction of blood-brain barrier permeability: A comparative analysis of central nervous system drugs versus secretase inhibitors for Alzheimer's disease” Curr. Opin. Drug Discov. Devel. 2006, 9, 303-313.] Finally, M4 Log BB model is with drug candidates that Log BB≥−1 [Vilar, S.; Chakrabarti, M.; Costanzi, S. “Prediction of passive blood-brain partitioning: Straightforward and effective classification models based on silico derived physicochemical descriptors” Journal of Molecular Graphics and Modelling 2010, 28, 899-903.]. Considering all computed parameters (Log P, PSA; M3 Log BB, log S, and at some extend CNS) together for studied compounds best physicochemical properties are for HR49, HR50, HR51, and HR59 (FIG. 45).

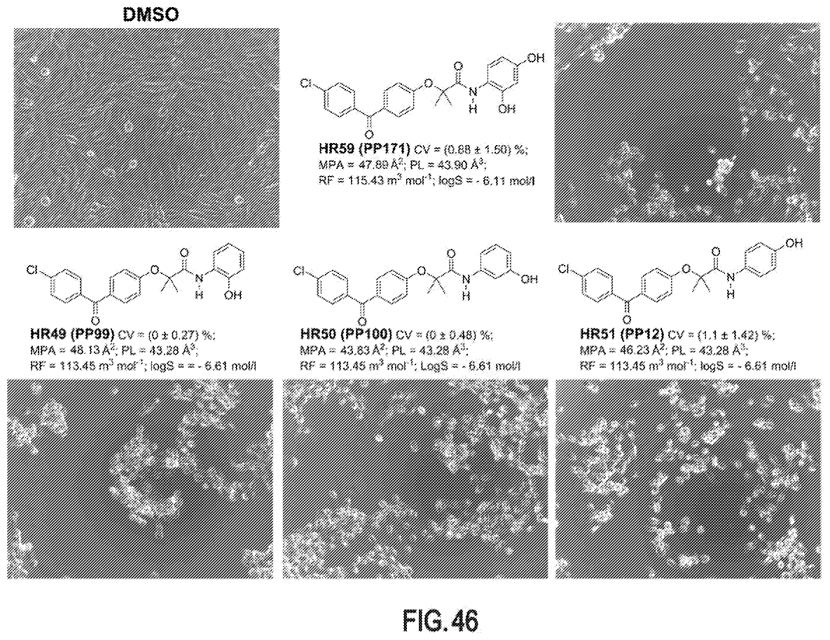

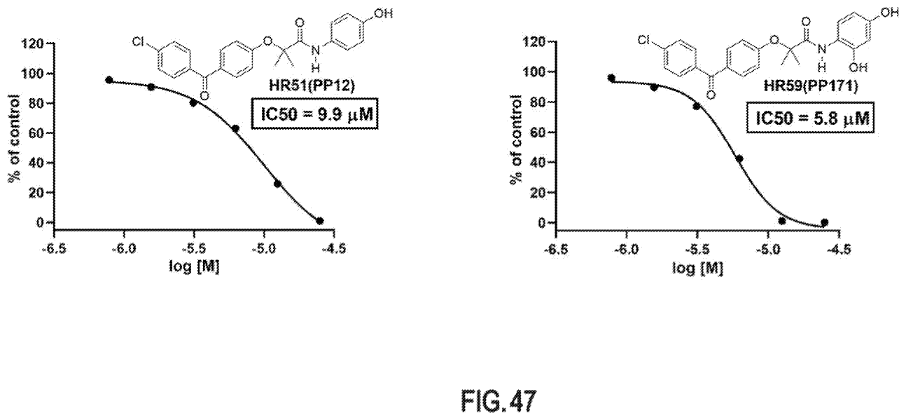

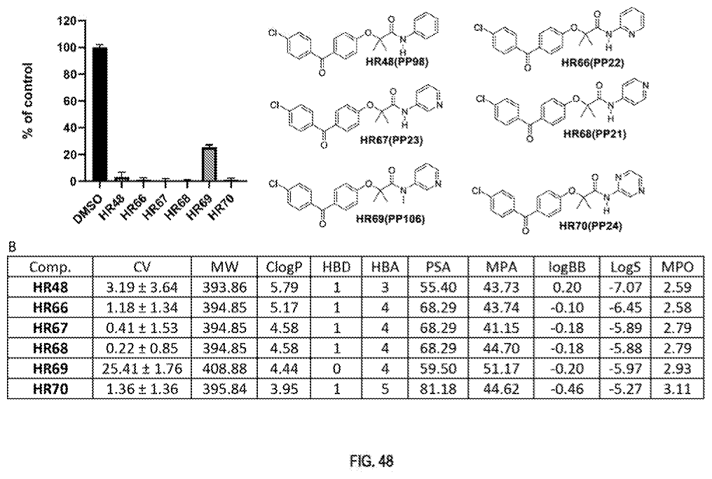

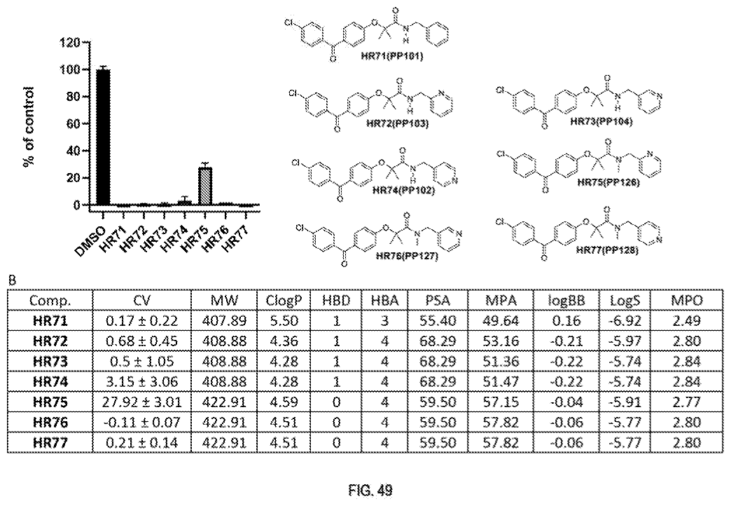

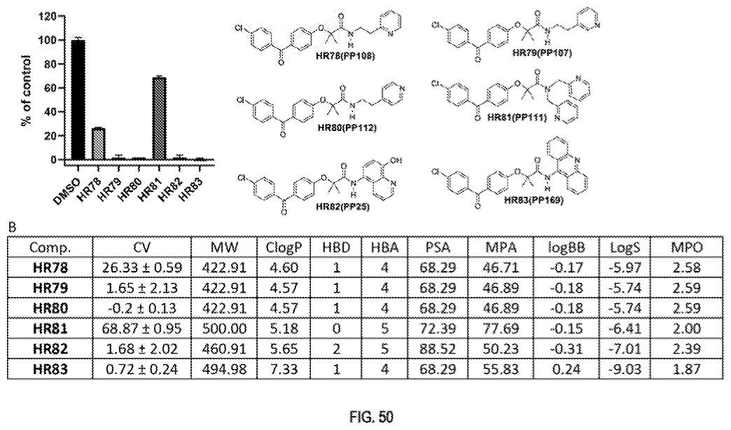

In conclusion, although there nine compounds from eighteen selected by from computational studies that have acceptable anti-glioblastoma activity on LN229 cell line at 25 M concentration only four (HR49, HR50, HR51, and HR59) are selected as drug candidates. This is in addition to recently reported BPA based compounds (HR28, HR32, HR37, and HR46) [Stalinska, J H.; Houser, L.; Rak, M.; Colley, S. B.; Reiss, K.; Jursic, B. S. “Exploring anticancer activity of structurally modified benzylphenoxyacetamide (BPA); I: Synthesis strategies and computational analyses of substituted BPA variants with high anti-glioblastoma potential” Scientific Reports 2019, 9, 17021.]. All of these compounds have strong in vitro anti-glioblastoma activity in a combination of physical properties that may contribute to the improved brain tumor penetration (FIG. 45). The IC₅₀ concentration of these compounds is below 10 μM, which is an acceptable therapeutic concentration for most clinically relevant anticancer drugs (FIG. 46). By exploring the computed structural variations of phenolic benzylphenoxyacetamide (BPA), along with testing of cell viability, it was demonstrated that presence of the phenol moiety is improving anti-glioblastoma activity (cell viability) with acceptable Log BB and Log S properties. We have also demonstrated that we can further tailored the molecular skeleton of BPA to obtain new drug candidate with desirable physicochemical properties such as brain penetration, and water solubility without diminishing cell viability. However, replacing the phenol with rather larger naphthol might not necessarily decrease the anti-glioblastoma activity (cell viability) but will result in decreased bioavailability [for instance see: Daham, A.; Miller, J. M. “The Solubility-Permeability Interplay and Its Implications in Formulation Design and Development for Poorly Soluble Drugs” the AAPS Journal 2012, 14, 244-251] and therefore these compounds are less probable to be consider as anti-glioblastoma candidates. Without wishing to be bound by theory, further exploring the phenolic BPAs in regard their experimental and computational predictors for blood-brain distribution and brain penetration will most likely produce new strong anti-glioblastoma drug candidates.

Methods

All starting materials were reagent grade and purchased from Sigma-Aldrich, ArkPharm, and TCI America. ¹H-NMR spectra were recorded on Varian Mercury 300 and Varian Mercury 400 Plus instruments in CDCl₃ and DMSO-d₆, using the solvent chemical shifts as an internal standard. All computed molecular descriptors were generated by ChemAxon MarvinSketch version 19.20. Electrostatic potential maps were calculated with PM3 semi-empirical method as implemented in Spartan '18 v 1.1.0. ¹H-NMR and ¹³C-NMR spectra for all HR compounds generated in this study are included in Supplementary Materials.

Cell culture and viability assays. Human glioblastoma LN-229 cells were maintained as semi-confluent monolayer culture in DMEM with 1 g/L glucose, sodium pyruvate, L-glutamine (Corning) supplemented with 10% heat inactivated FBS (Gibco) and P/S (50 units/mL of penicillin and 50 μg/mL of streptomycin) at 37° C. in a 5% CO2 atmosphere. Prior to treatment, cells were plated well in 96-well plates (BD Falcon) at initial density of 2×10³ cells/cm². Stock solutions of the compounds were prepared in DMSO, diluted in cell culture medium and added to the cells in triplicate for every experimental condition 24 h after plating (final concentration 25 μM). For the vehicle control, DMSO was used at 0.5%. MTT assay⁴⁰ (measuring cell metabolic activity) was performed after a 72 h incubation in the presence of the compounds as previously described. Following 1 h incubation with MTT, formazan crystals were dissolved in 5 mM HCl in isopropanol and absorbance read at 540 nm. Data represent mean values expressed as percentage of vehicle control±SD. Phase contrast images of treated cells were taken 48 hours following the treatment with a BZ-X800 fluorescence microscope (Keyence) equipped with a 20× objective.

Method A (Larger scale preparation without extraction or crystallization). 2-(4-(4-chlorobenzoyl)phenoxy)-N-(2-hydroxyphenyl)-2-methylpropanamide (HR49). Freshly prepared fenofibric chloride (FFC) from FFA (9.6 g; 0.03 mol) and oxalyl chloride (5.2 ml; 7.6 g; 0.09 mol) in dichloromethane (50 ml) by stirring at room temperature overnight. After solvent evaporation and solid residue drying under argon FFC was dissolved in dichloromethane (50 ml) and mixed with pyridine (50 ml) solution of 2-aminophenol (2.6 g; 0.025 mol). Resulting solution was stirred at room temperature for 3 hours and then refluxed for additional 3 hours. Solvent was evaporated under reduced pressure and solid residue mixed with ethanol (100 ml) and refluxed with stirring until all solid material was dissolved. This clear alcohol solution was mixed with hot (70° C.) 3% sodium carbonate solution (400 ml) and refluxed for half an hour. Volume of the mixture was reduced to ˜½ by destiling off solvent at room temperature. Resulting mixture was left at room temperature for several hours. Resulting solid product was separated by filtration, extensively washed with water (10×100 ml) and dried at 110° C. for 1 hour. Isolated yield 97% (4.77 g). ¹H-NMR (DMSO-d₆, 400 MHz) δ 9.95 (1H, s, OH), 9.09 (1H, s, NH), 7.93 (1H, d, J=8.0 Hz), 7.73 (2H, d, J=8.0 Hz), 7.70 (2H, d, J=8.4 Hz), 7.59 (2H, d, J=8.4 Hz), 7.11 (2H, d, J=8.4 Hz), 6.23 (1H, t, J=7.6 Hz), 6.83 (1H, d, J=8.0 Hz), 6.78 (1H, t, J=8.0 Hz), and 1.60 (6H, s) ppm. ¹³C-NMR (DMSO-d₆) δ 193.8, 171.7, 158.9, 147.7, 137.6, 136.5, 132.2, 131.7, 131.3, 129.1, 126.1, 125.1, 121.2, 119.9, 119.6, 115.5, 82.3 and 25.3 ppm.

Method B (small scale preparation). Preparation of 2-(4-(4-chlorobenzoyl)phenoxy)-N-(2-hydroxy-5-methylphenyl)-2-methylpropanamide (HR52). Dichloromethane (10 ml) suspension of fenofibric acid (FFA) (191 mg; 0.6 mmol) and oxalyl chloride (0.2 ml; 384 mg; 2 mmol) was stirred at room temperature for five minutes. Few drops of N,N-dimethylformamide (DMF) was added and immediately bubbles start to form resulting for reaction mixture to become clear solution in approximately 30 minutes. This solution was stirred at 60° C. with slow solvent evaporation. Residue of solvent and oxalyl chloride was removed by drying under argon flow at room temperature. Resulting yellow solid was dissolved in dichloromethane (10 ml) and mixed with 2-amino-4-methylphenol (62 mg; 0.5 mmol) in THF (10 ml) and Na₂CO₃ (1.06 g; 10 mmol) in water (10 ml). Resulting mixture was stirred at room temperature for five hours. Solvent was evaporated under reduced pressure and solid residue was mixed with dichloromethane (50 ml) and water (50 ml). Resulting mixture was stirred with sonication at room temperature until all solid was dissolved. Water layer was discarded, and dichloromethane layer was washed with 5% Na₂CO₃ (3×50 ml), water (50 ml), 5% HCl (3×50 ml), water (50 ml) and dried over anhydrous Na₂CO₃. After solvent evaporation product was purified by crystallization from dichloromethane (˜3 ml) and hexane (20 ml). Isolated yield 90% (190 mg). ¹H-NMR (DMSO-d₆, 400 MHz) δ 9.74 (1H, broad s, OH), 9.06 (1H, s, NH), 7.81 (1H, s), 7.73 (2H, d, J=8.8 Hz), 7.70 (2H, d, J=8.8 Hz), 7.58 (2H, d, J=8.8 Hz), 7.11 (2H, d, J=8.4 Hz), 6.73 (2H, s), 2.18 (3H, s), and 1.60 (6H, s) ppm. ¹³C-NMR (DMSO-d₆) δ 193.8, 171.6, 158.8, 145.4, 137.7, 136.5, 132.2, 131.7, 131.3, 129.1, 128.1, 125.9, 125.3, 121.5, 119.9, 115.2, 82.2, 25.3, and 20.9 ppm.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. An anticancer compound comprising the following formula: 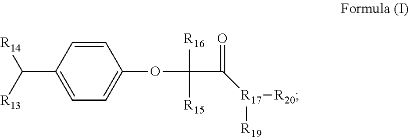
wherein,
R₁₃ is selected from the group consisting of substituted cycloalkyl, aryl, substituted aryl, substituted heterocycle, and substituted aromatic heterocycle;
R₁₄ is selected from the group consisting of hydrogen and double bonded oxygen;
R₁₅ is selected from the group consisting of hydrogen, substituted alkyl, and substituted aryl;
R₁₆ is selected from the group consisting of hydrogen, substituted alkyl, and substituted aryl;
R₁₇ is selected from the group consisting of nitrogen and carbon;
R₁₉ is selected from the group consisting of hydrogen, hydroxy, amino, substituted amino, alkyl, hydroxyalkyl, cycloalkyl, heterocycle, aryl, hydroxyaryl, aromatic heterocycle, and hydroxyaryl; and
R₂₀ is selected from the group consisting of hydrogen, hydroxy, amino, substituted amino, alkyl, hydroxyalkyl, cycloalkyl, heterocycle, aryl, hydroxyaryl, aromatic heterocycle, and hydroxyaryl.

2. The anticancer compound of claim 1, wherein the anticancer compound comprises the following formula: 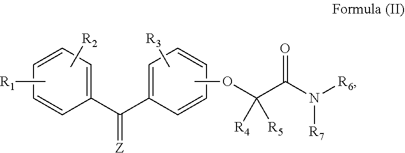
wherein,
R₁ is selected from the group consisting of hydrogen, halogen, alkyl, oxyalkyl, aryl, oxyaryl, halogen, cyano, nitro, carboxyl, alkoxy, and carboxyalkyl;
R₂ is selected from the group consisting of hydrogen, halogen, alkyl, oxyalkyl, aryl, oxyaryl, halogen, cyano, nitro, carboxyl, alkoxy and carboxyalkyl;
R₃ is selected from the group consisting of hydrogen, halogen, alkyl, oxyalkyl, aryl, oxyaryl, halogen, cyano, nitro, carboxyl, alkoxy, and carboxyalkyl;
R₄ is selected from the group consisting of hydrogen, alkyl, and substituted alkyl;
R₅ is selected from the group consisting of hydrogen, alkyl, and substituted alkyl;
R₆ is selected from the group consisting of hydrogen, hydroxy, amino, substituted amino, alkyl, hydroxyalkyl, cycloalkyl, heterocycle, aryl, hydroxyaryl, aromatic heterocycle, and hydroxyaryl;
R₇ is selected from the group consisting of hydrogen, hydroxy, amino, substituted amino, alkyl, hydroxyalkyl, cycloalkyl, heterocycle, aryl, hydroxyaryl, aromatic heterocycle, hydroxyaryl, pyrrolidine, piperidine, morpholine, pyrrole, and an alkyl substituent thereof; and
Z is selected from the group consisting of H₂, O, S, heterocycle, aryl, NNR₂ wherein R is selected from the group consisting of alkyl, aryl, and heterocycle.

3. The anticancer compound of claim 2, wherein the anticancer compound comprises the following structures: 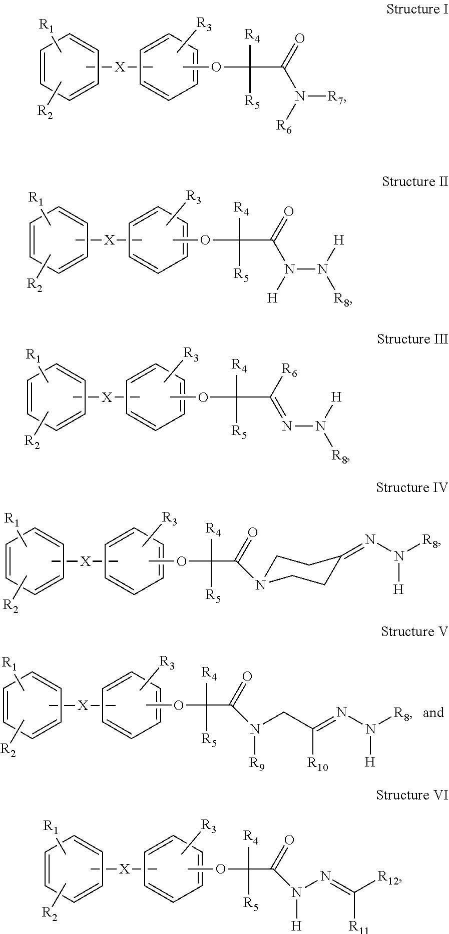
wherein, R₁, R₂, R₃ is selected from the group consisting of H, alkyl, O-alkyl, nitro, cyano, and halogen;
R₄, R₅, R₆, R₉ is selected from the group consisting of H and alkyl;
R₇ is selected from the group consisting of alkyl, substituted alkyl, hydroxy alkyl, aryl, substituted aryl, heteroaryl, substituted heteroaryl, hydroxy, and amino;
R₈ is selected from the group consisting of aryl, substituted aryl, heteroaryl, substituted heteroaryl, CO-aryl, CO-heteroaryl, and CO-linker;
R₁₀ is selected from the group consisting of alkyl, hydroxyalkyl, substituted aryl, and substituted heteroaryl;
R₁₁, R₁₂ is selected from the group consisting of H, alkyl, aryl, heteroaryl, alkyl with linker, and aryl with linker;
X is selected from the group consisting of CO, CH₂, O, CC, and CH═CH; or
any combination thereof.

4.-9. (canceled)

10. The anticancer compound of claim 1, wherein the anticancer compound comprises any one of the structures of Table 2.

11. The anticancer compound of claim 10, wherein the anticancer compound comprises any one of the following structures: 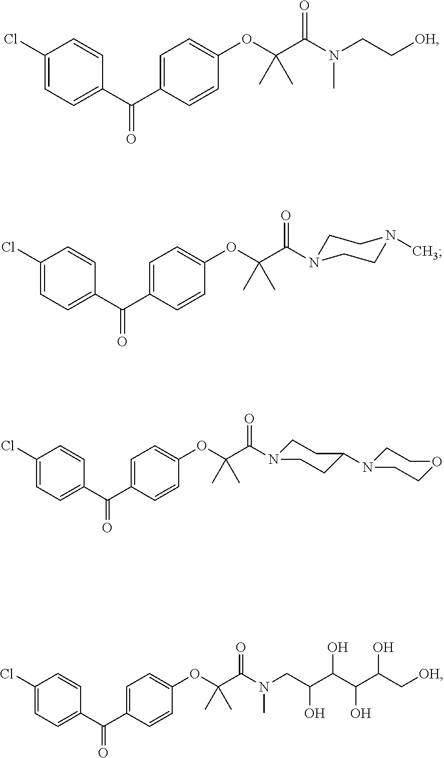 [SMILES: CN(CCO)C(=O)C(C)(C)Oc1ccc(C(=O)c2ccc(Cl)cc2)cc1 ; CN1CCN(C(=O)C(C)(C)Oc2ccc(C(=O)c3ccc(Cl)cc3)cc2)CC1 ; CC(C)(Oc1ccc(C(=O)c2ccc(Cl)cc2)cc1)C(=O)N1CCC(N2CCOCC2)CC1 ; CN(CC(O)C(O)C(O)C(O)CO)C(=O)C(C)(C)Oc1ccc(C(=O)c2ccc(Cl)cc2)cc1]
pharmaceutically acceptable salt thereof, or a derivative thereof.

12. The anticancer compound of claim 2, comprising the following formula: 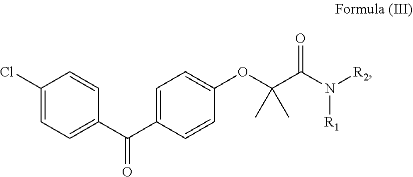
wherein R₁ is selected from the group consisting of H, alkyl, hydroxyalkyl;
R₂ is selected from the group consisting of H, alkyl, hydroxyalkyl, polyhydroxyalkyl, aminoalkyl, dialkylaminoalkyl; or
wherein R₁-R₂ is selected from the group consisting of (CH₂)ₙ, (CH₂)ₙO(CH₂)ₘ, (CH₂)ₙCHOH(CH₂)ₘ, and (CH₂)ₙNCH₃(CH₂)ₘ, wherein n and m are 1, 2, 3, 4, or 5.

13. (canceled)

14. The anticancer compound of claim 12, wherein the anticancer compound comprises any one of the following structures: 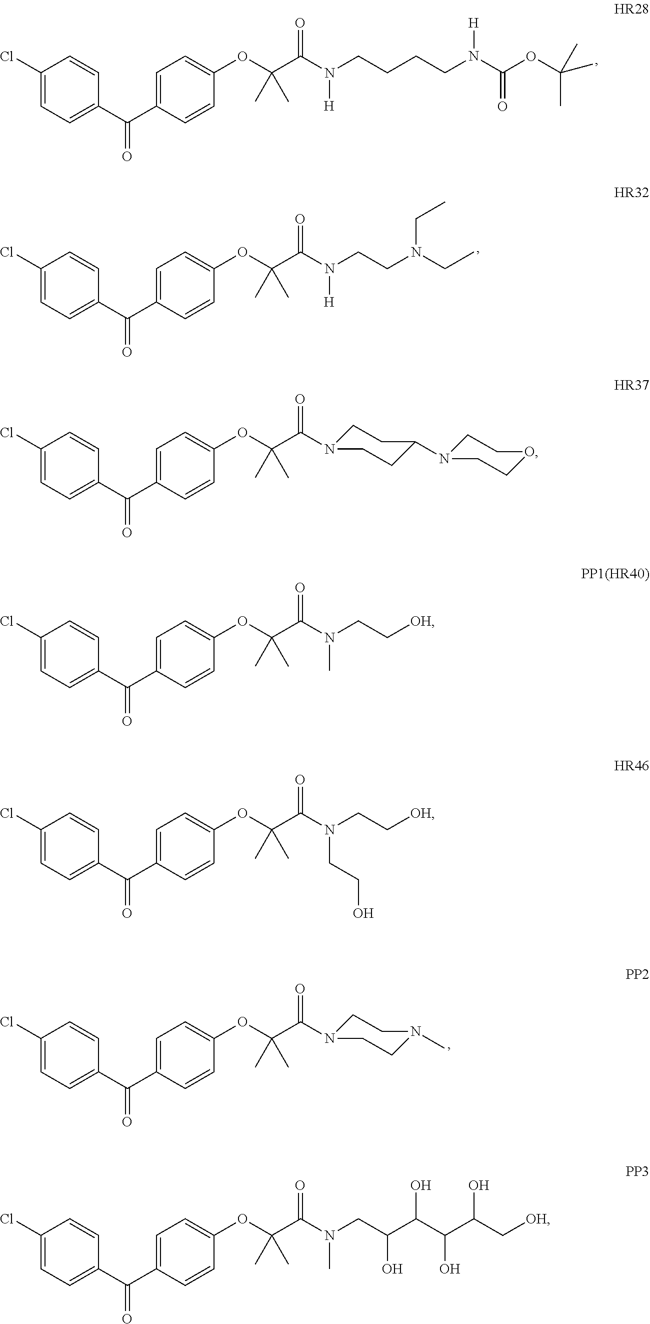
a pharmaceutically acceptable salt thereof, or a derivative thereof.

15. The anticancer compound of claim 2 comprising the following formula: 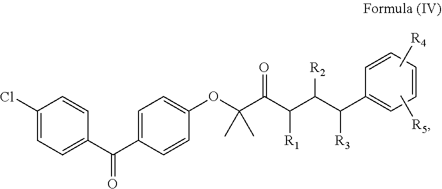
wherein R₁ is selected from the group consisting of H and CH₃;
R₂ is selected from the group consisting of OH and OCH₃;
R₃ is selected from the group consisting of OH and OCH₃;
R₄ is selected from the group consisting of OH and OCH₃;
R₅ is selected from the group consisting of OH and OCH₃;
or any combination thereof.

16. (canceled)

17. The anticancer compound of claim 15, wherein the anticancer compound comprises any one of the following structures: 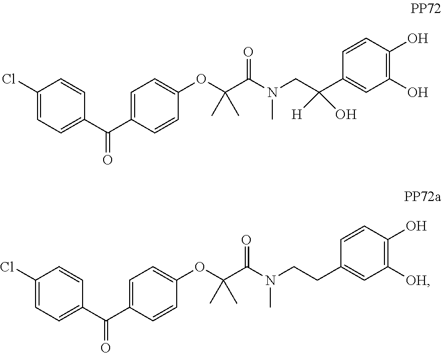 [SMILES: CN(CC(O)c1ccc(O)c(O)c1)C(=O)C(C)(C)Oc1ccc(C(=O)c2ccc(Cl)cc2)cc1 ; CN(CCc1ccc(O)c(O)c1)C(=O)C(C)(C)Oc1ccc(C(=O)c2ccc(Cl)cc2)cc1]
pharmaceutically acceptable salt thereof, or a derivative thereof.

18. The anticancer compound of claim 2 comprising the following formula: 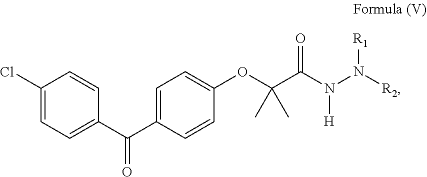
wherein R₁ is selected from the group consisting of H, CH₃, and (CH₂)₅; and
wherein R₂ is selected from the group consisting of H, CH₃, and (CH₂)₅.

19. (canceled)

20. The anticancer compound of claim 18, wherein the anticancer compound comprises any one of the following structures: 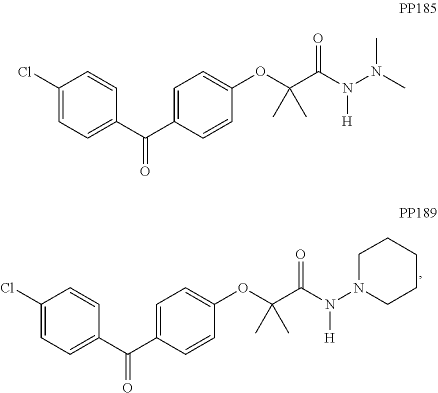 [SMILES: CN(C)NC(=O)C(C)(C)Oc1ccc(C(=O)c2ccc(Cl)cc2)cc1 ; CC(C)(Oc1ccc(C(=O)c2ccc(Cl)cc2)cc1)C(=O)NN1CCCCC1]
a pharmaceutically acceptable salt thereof, or a derivative thereof.

21. (canceled)

22. (canceled)

23. The anticancer compound of claim 2, wherein the anticancer compound comprises any one of the following structures: 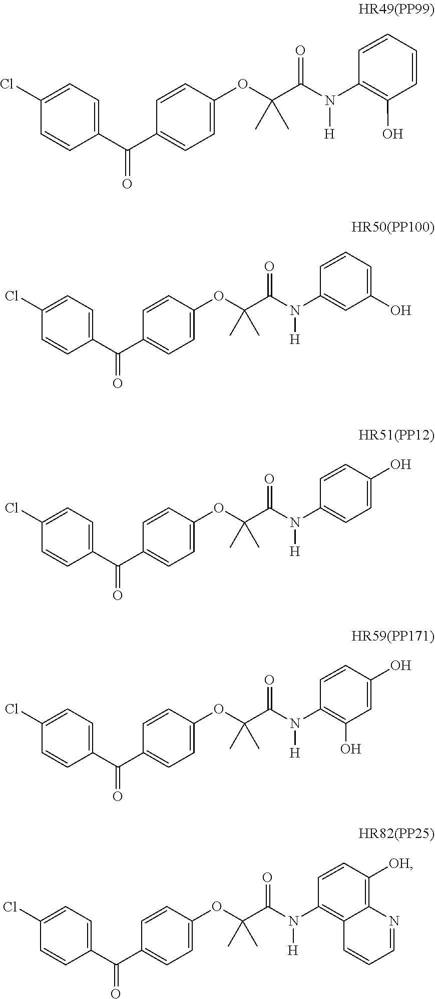 [SMILES: CC(C)(Oc1ccc(C(=O)c2ccc(Cl)cc2)cc1)C(=O)Nc1ccccc1O ; CC(C)(Oc1ccc(C(=O)c2ccc(Cl)cc2)cc1)C(=O)Nc1cccc(O)c1 ; CC(C)(Oc1ccc(C(=O)c2ccc(Cl)cc2)cc1)C(=O)Nc1ccc(O)cc1 ; CC(C)(Oc1ccc(C(=O)c2ccc(Cl)cc2)cc1)C(=O)Nc1ccc(O)cc1O ; CC(C)(Oc1ccc(C(=O)c2ccc(Cl)cc2)cc1)C(=O)Nc1ccc(O)c2ncccc12]
a pharmaceutically acceptable salt thereof, or a derivative thereof.

24. (canceled)

25. (canceled)

26. The anticancer compound of claim 2, wherein the anticancer compound comprises any one of the following structures: 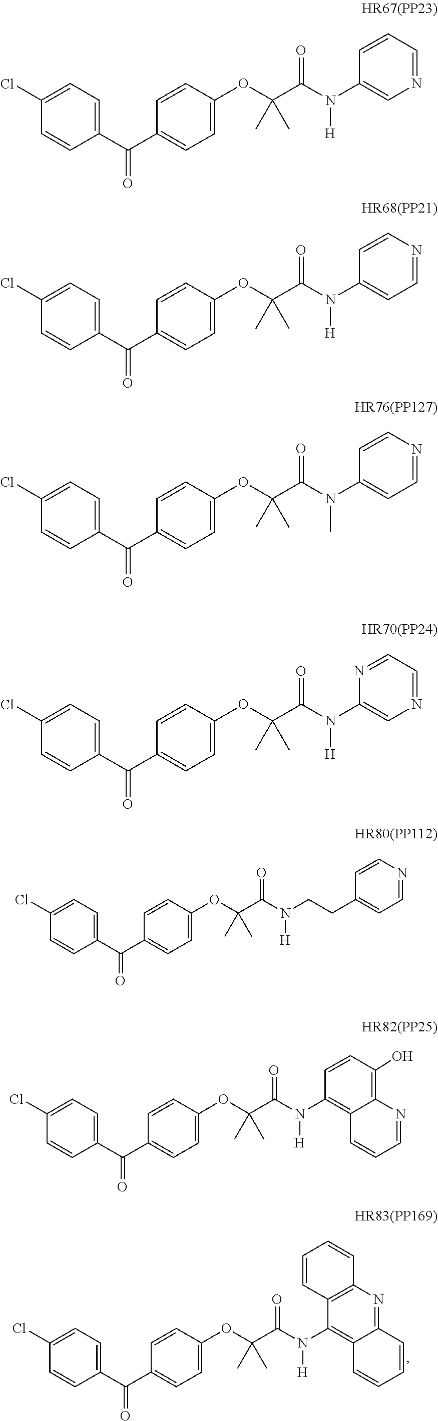
a pharmaceutically acceptable salt thereof, or a derivative thereof.

27. (canceled)

28. (canceled)

29. The anticancer compound of claim 2, wherein the anticancer compound comprises any one of the following structures: 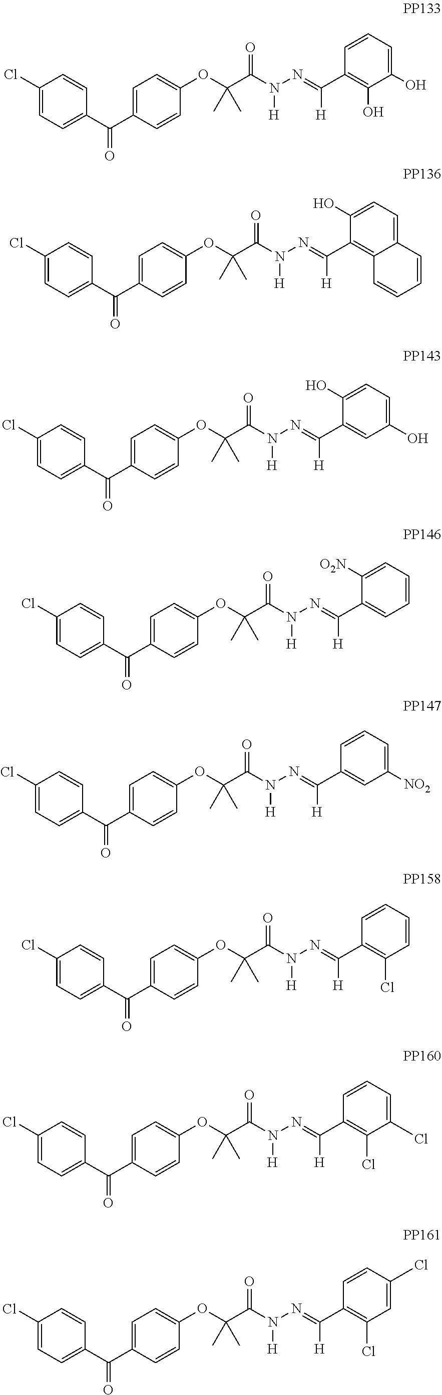 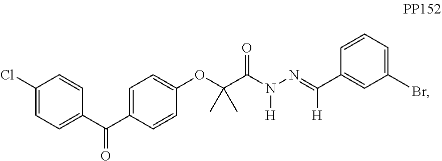 [SMILES: CC(C)(Oc1ccc(C(=O)c2ccc(Cl)cc2)cc1)C(=O)N/N=C/c1cccc(Br)c1]
a pharmaceutically acceptable salt thereof, or a derivative thereof.

30. The anticancer compound of claim 2, wherein the compound comprises the following formula: 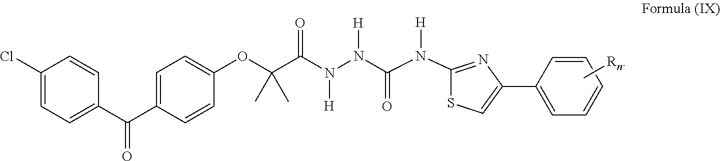
wherein, Rn is selected from the group consisting of H, Cl, and Br; and
wherein n is selected from the group consisting of 0, 1, 2, and 3.

31. (canceled)

32. The anticancer compound of claim 30, wherein the anticancer compound comprises any one of the following structures: 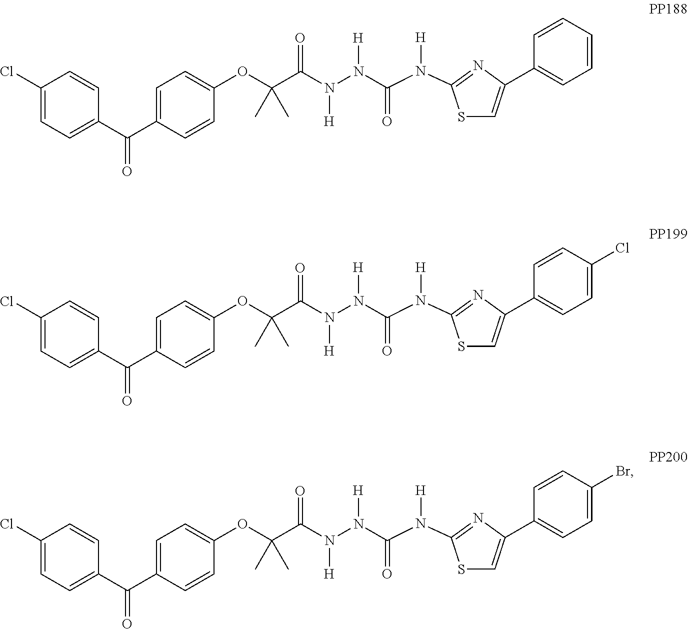 [SMILES: CC(C)(Oc1ccc(C(=O)c2ccc(Cl)cc2)cc1)C(=O)NNC(=O)Nc1nc(-c2ccccc2)cs1 ; CC(C)(Oc1ccc(C(=O)c2ccc(Cl)cc2)cc1)C(=O)NNC(=O)Nc1nc(-c2ccc(Cl)cc2)cs1 ; CC(C)(Oc1ccc(C(=O)c2ccc(Cl)cc2)cc1)C(=O)NNC(=O)Nc1nc(-c2ccc(Br)cc2)cs1]
a pharmaceutically acceptable salt thereof, or a derivative thereof.

33. The anticancer compound of claim 2, wherein the compound comprises the following formula: 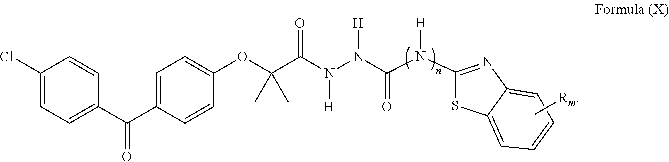
wherein R is selected from the group consisting of H, Cl and Br; and
wherein n is H or 1; and
wherein m is 1, 2, or 3.

34. (canceled)

35. The anticancer compound of claim 33, wherein the anticancer compound comprises any one of the following structures: 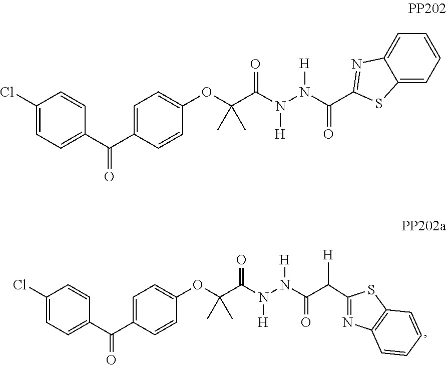 [SMILES: CC(C)(Oc1ccc(C(=O)c2ccc(Cl)cc2)cc1)C(=O)NNC(=O)c1nc2ccccc2s1 ; CC(C)(Oc1ccc(C(=O)c2ccc(Cl)cc2)cc1)C(=O)NNC(=O)Cc1nc2ccccc2s1]
pharmaceutically acceptable salt thereof, or a derivative thereof.

36. A pharmaceutical composition comprising an anticancer compound of claim 1 and a pharmaceutically acceptable carrier and/or pharmaceutically acceptable excipient.

37. The pharmaceutical composition of claim 36, wherein the pharmaceutically acceptable carrier is albumin.

38. (canceled)

39. A method of synthesizing an anticancer compound of claim 3, wherein the method comprises a schematic as indicated in FIG. 15.

40. The method of claim 39, wherein the method comprises synthesizing
2-[4-(4-chlorobenzoyl)phenoxy]-N-(2-hydroxyethyl)-N,2-dimethylpropanamide (PP1), comprising:
Preparing a dichloromethane (20 ml) suspension of fenofibric acid (318.75 mg; 1 mmol) and oxalyl chloride (0.25 mL; 380.1 mg; 3 mmol)
adding one drop of N,N-dimethylformamide while stirring at room temperature for 5 hours;
evaporating solvent under reduced pressure;
resolving the residue in dichloromethane (10 ml) and again evaporating to the solid residue;
dissolving the solid residue in dichloromethane (20 ml);
stirring with dichloromethane (10 ml) solution at room temperature and adding 2-(methylamino)ethanol (0.24 ml; 225 mg; 3 mmol);
stirring the reaction mixture at room temperature;
washing the reaction mixture with water (3×15 ml), 5% hydrochloric acid (3×15 ml), water (3×15 ml), 10% sodium carbonate (3×15 ml), and water (3×15 ml) and drying over anhydrous sodium carbonate; evaporating solvent under reduced pressure;
crystalizing the product at room temperature;
and washing with hexane.

41. The method of claim 39, wherein the method comprises synthesizing
2-[4-(4-chlorobenzoyl)phenoxy]-N-(2-hydroxyethyl)-N,2-dimethylpropanamide (PP1), the comprising:
stirring a dichloromethane (100 ml) solution of fenofibric acid (637 mg; 2 mmol), 1-ethyl-3-(3-dimethylaminopropyl)carbodiimide, hydrochloride (EDC, 576; 3 mmol), and 2-(methylamino)ethanol (600 mg; 8 mmol) at room temperature overnight washing a dichloromethane solution with 5% hydrochloric acid (5×20 ml), water (5×20 ml), 10% sodium carbonate (5×20 ml), water (3×20 ml) and drying over anhydrous sodium carbonate;
evaporating solvent; and
crystalizing a product.

42. The method of claim 39 comprising synthesizing 2-[4-(4-chlorobenzoyl)phenoxy]-2-methyl-1-(4-methylpiperazin-1-yl)propan-1-one (PP2), comprising:
stirring a dichloromethane (30 ml) solution of fenofibric chloride (1 mmol into a water (5 ml) solution of sodium carbonate (216 mg; 2 mmol) with tetrahydrofuran (10 ml) and of 1-methylpiperazine (0.13 ml; 120 mg; 1.2 mmol) and stirring for about 1 hour; adding water (30 ml) and separating an organic layer; washing with water (3×10 ml), 5% hydrochloric acid (3×10 ml), 10% sodium carbonate (3×10 ml), water and drying over anhydrous sodium carbonate; crystalizing a product.

43. The method of claim 39, comprising synthesizing 2-[4-(4-chlorobenzoyl)phenoxy]-N,2-dimethyl-N-[(2S,3R,4R,5R)-2,3,4,5,6-pentahydroxyhexyl]propenamide (PP3), comprising: dissolving fenofibric acid chloride in dichloromethane (15 ml) and mixing with acetonitrile (20 ml) and water (10 ml) solution of N-methyl-D-glucamine (196 mg; 1 mmol) and sodium carbonate (212 mg; 2 mmol);
stirring at room temperature for five minutes; evaporating solvent and mixing with dichloromethane (50 ml) and water (20 ml);
separating the dichloromethane layer washing with 10% sodium carbonate (3×10 ml), water (3×10 ml), drying over anhydrous sodium carbonate; and
evaporating the solvent and washing with hexane.

44. The method of claim 39, comprising synthesizing 2-[4-(4-chlorobenzoyl)phenoxy]-2-methyl-1-[4-(morpholin-4-yl)piperidin-1-yl]propan-1-one (PP4), comprising: dissolving fenofibric acid chloride in dichloromethane (30 ml) and mixing with a tetrahydrofuran (10 ml) solution of 4-morpholinopiperidine (100 mg; 0.6 mol) and a water (10 ml) solution of sodium carbonate (106 mg; 1 mmol) and stirring for one hour at room temperature;
adding water (20 ml) and separating the organic layer;
washing with 5% hydrochloric acid (5×20 ml), water (3×10 ml), 10% sodium carbonate (5×20 ml) and again with water (3×10 ml);
drying over anhydrous sodium carbonate solvent and evaporating to give product.

45. The method of claim 39, comprising synthesizing 4-(1-{2-[4-(4-chlorobenzoyl)phenoxy]-2-methylpropanoyl}piperidin-4-yl)morpholin-4-iumchloride (PP4HCl), comprising:
mixing concentrated hydrochloric acid (2 ml) and PP4 (94 mg; 0.2 mmol) and sonicating at room temperature; evaporating to dryness; dissolving in dry ether evaporating the dry ethyl ether.

46. A method of treating a subject afflicted with a disease, the method comprising administering to the subject afflicted with cancer a therapeutically effective amount of the pharmaceutical composition of claim 36.

47. The method of claim 46, wherein the disease comprises a brain cancer or a blood cancer.

48. The method of claim 46, wherein the cancer comprises a solid tumor or a liquid cancer.

49. The method of claim 47, wherein the brain cancer comprises a glioma.

50. The method of claim 49, wherein the glioma comprises an astrocytoma, an ependymoma, or an oligodendroglioma.

51. The method of claim 49, wherein the glioma comprises glioblastoma.

52. The method of claim 47, wherein the blood cancer comprises leukemia, lymphoma, and hemangiosarcoma.

53. A method of attenuating abnormal cell proliferation, the method comprising administering to the subject in need thereof a therapeutically effective amount of the pharmaceutical composition of claim 36, wherein the composition attenuates abnormal cell proliferation.

54. The method of claim 53, wherein the cell comprises a cancer cell.

55. A method of inhibiting or delaying metastatic invasion of a cancer cell, the method comprising administering to the subject in need thereof a therapeutically effective amount of the pharmaceutical composition of claim 36, wherein the composition inhibits or delays metastatic invasion of a cancer cell.

56. The method of claim 46, wherein the pharmaceutical composition is administered orally to the subject.

57. The method of claim 54, wherein the cancer cell comprises a primary cancer cell.

58. The method of claim 54, wherein the cancer cell is a brain cancer cell or a blood cancer cell.

59. The method of claim 54, wherein the cancer comprises a solid tumor or a liquid cancer.

60. The method of claim 58, wherein the brain cancer comprises a glioma.

61. The method of claim 60, wherein the glioma comprises an astrocytoma, an ependymoma, or an oligodendroglioma.

62. The method of claim 60, wherein the glioma comprises glioblastoma.

63. The method of claim 58, wherein the blood cancer comprises leukemia, lymphoma, and hemangiosarcoma.
